SEQUENCE LISTING (445 sequences)

SEQ ID NO: 1 - DNA, 31 bp, synthetic construct
  source 1..31: mol_type = other DNA
agagggtatataatggaagctcgacttccag

SEQ ID NO: 2 - DNA, 52 bp, synthetic construct
  source 1..52: mol_type = other DNA
gggaatttccggggactttccgggaatttccggggactttccgggaatttcc

SEQ ID NO: 3 - DNA, 85 bp, synthetic construct
  source 1..85: mol_type = other DNA
caccagacagtgacgtcagctgccagatcccatggccgtcatactgtgacgtctttcagacaccccattgacgtcaatgggagaa

SEQ ID NO: 4 - DNA, 90 bp, synthetic construct
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 5 - DNA, 115 bp, synthetic construct
  source 1..115: mol_type = other DNA
aggatgtccatattaggacatctaggatgtccatattaggacatctaggatgtccatattaggacatctaggatgtccatattaggacatctaggatgtccatattaggacatct

SEQ ID NO: 6 - DNA, 115 bp, synthetic construct
  source 1..115: mol_type = other DNA
agtatgtccatattaggacatctaccatgtccatattaggacatctactatgtccatattaggacatcttgtatgtccatattaggacatctaaaatgtccatattaggacatct

SEQ ID NO: 7 - DNA, 48 bp, synthetic construct
  source 1..48: mol_type = other DNA
tgagtcagtgactcagtgagtcagtgactcagtgagtcagtgactcag

SEQ ID NO: 8 - DNA, 120 bp, synthetic construct
  source 1..120: mol_type = other DNA
agatcaaagggtttaagatcaaagggcttaagatcaaagggtataagatcaaagggcctaagatcaaagggactaagatcaaagggtttaagatcaaagggcttaagatcaaagggccta

SEQ ID NO: 9 - DNA, 32 bp, synthetic construct
  source 1..32: mol_type = other DNA
gtctagacgtctagacgtctagacgtctagac

SEQ ID NO: 10 - DNA, 24 bp, synthetic construct
  source 1..24: mol_type = other DNA
cagacacagacacagacacagaca

SEQ ID NO: 11 - DNA, 65 bp, synthetic construct
  source 1..65: mol_type = other DNA
ggatccggtactcgagatctgcgatctaagtaagcttggcattccggtactgttggtaaagccac

SEQ ID NO: 12 - DNA, 588 bp, synthetic construct
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 13 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 14 - DNA, 544 bp, synthetic construct
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 15 - DNA, 399 bp, synthetic construct
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 16 - DNA, 511 bp, synthetic construct
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 17 - DNA, 408 bp, synthetic construct
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 18 - DNA, 344 bp, synthetic construct
  source 1..344: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 19 - DNA, 1229 bp, synthetic construct
  source 1..1229: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 20 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 21 - DNA, 1718 bp, synthetic construct
  source 1..1718: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 22 - DNA, 212 bp, synthetic construct
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 23 - DNA, 2379 bp, synthetic construct
  source 1..2379: mol_type = other DNA
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 24 - DNA, 529 bp, synthetic construct
  source 1..529: mol_type = other DNA
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 25 - DNA, 794 bp, synthetic construct
  source 1..794: mol_type = other DNA
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 26 - DNA, 532 bp, synthetic construct
  source 1..532: mol_type = other DNA
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 27 - DNA, 701 bp, synthetic construct
  source 1..701: mol_type = other DNA
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 28 - DNA, 465 bp, synthetic construct
  source 1..465: mol_type = other DNA
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 29 - DNA, 538 bp, synthetic construct
  source 1..538: mol_type = other DNA
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 30 - DNA, 1091 bp, synthetic construct
  source 1..1091: mol_type = other DNA
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 31 - DNA, 66 bp, synthetic construct
  source 1..66: mol_type = other DNA
atgtgtcaccagcagctcgttatatcctggtttagtttggtgtttctcgcttcacccctggtggca

SEQ ID NO: 32 - DNA, 66 bp, synthetic construct
  source 1..66: mol_type = other DNA
atgtgccatcagcaactcgtcatctcctggttctcccttgtgttcctcgcttcccctctggtcgcc

SEQ ID NO: 33 - DNA, 42 bp, synthetic construct
  source 1..42: mol_type = other DNA
atgcaactgctgtcatgtatcgcactcatcctggcgctggta

SEQ ID NO: 34 - DNA, 60 bp, synthetic construct
  source 1..60: mol_type = other DNA
atgtatcggatgcaacttttgagctgcatcgcattgtctctggcgctggtgacaaattcc

SEQ ID NO: 35 - DNA, 45 bp, synthetic construct
  source 1..45: mol_type = other DNA
atgaatctcttgctcatacttacgtttgtcgctgctgccgttgcg

SEQ ID NO: 36 - DNA, 51 bp, synthetic construct
  source 1..51: mol_type = other DNA
atgggcgtgaaggtcttgtttgcccttatctgcatagctgttgcggaggcg

SEQ ID NO: 37 - DNA, 72 bp, synthetic construct
  source 1..72: mol_type = other DNA
atgccgatggggagccttcaacctttggcaacgctttatcttctggggatgttggttgctagttgccttggg

SEQ ID NO: 38 - DNA, 63 bp, synthetic construct
  source 1..63: mol_type = other DNA
atggaaactgacacgttgttgctgtgggtattgctcttgtgggtcccaggatctacgggcgac

SEQ ID NO: 39 - DNA, 66 bp, synthetic construct
  source 1..66: mol_type = other DNA
atggatatgagggttcccgcccagcttttggggctgcttttgttgtggcttcgaggggctcggtgt

SEQ ID NO: 40 - DNA, 48 bp, synthetic construct
  source 1..48: mol_type = other DNA
atgaagtgtctgttgtacctggcgtttctgttcattggtgtaaactgt

SEQ ID NO: 41 - DNA, 84 bp, synthetic construct
  source 1..84: mol_type = other DNA
atgaatatcaaaggaagtccgtggaagggtagtctcctgctgctcctcgtatctaaccttctcctttgtcaatccgtggcaccc

SEQ ID NO: 42 - DNA, 54 bp, synthetic construct
  source 1..54: mol_type = other DNA
atgaaatgggtaacattcatatcacttctctttctgttcagctctgcgtattct

SEQ ID NO: 43 - DNA, 57 bp, synthetic construct
  source 1..57: mol_type = other DNA
atgacaaggcttactgttttggctctcctcgctggactcttggcttcctcccgagca

SEQ ID NO: 44 - DNA, 51 bp, synthetic construct
  source 1..51: mol_type = other DNA
atgagggcttggattttttttctgctctgccttgccggtcgagccctggcg

SEQ ID NO: 45 - DNA, 48 bp, synthetic construct
  source 1..48: mol_type = other DNA
atgcctcttctgcttttgcttcctcttttgtgggcaggtgccctcgca

SEQ ID NO: 46 - DNA, 87 bp, synthetic construct
  source 1..87: mol_type = other DNA
atgaactctttctcaacctctgcgtttggtccggtcgctttctcccttgggctcctgcttgtcttgccagcagcgtttcctgcgcca

SEQ ID NO: 47 - DNA, 60 bp, synthetic construct
  source 1..60: mol_type = other DNA
atgacaagtaaactggcggtagccttgctcgcggcctttttgatttccgcagccctttgt

SEQ ID NO: 48 - DNA, 69 bp, synthetic construct
  source 1..69: mol_type = other DNA
atgaaggtaagtgcagcgttgctttgccttctcctcattgcagcgacctttattcctcaagggctggcc

SEQ ID NO: 49 - DNA, 78 bp, synthetic construct
  source 1..78: mol_type = other DNA
atgggagcggcagctagaacacttcgacttgcccttgggctcttgctccttgcaaccctccttagacctgccgacgca

SEQ ID NO: 50 - DNA, 63 bp, synthetic construct
  source 1..63: mol_type = other DNA
atgtcaccgttgttgcggagattgctgttggccgcacttttgcaactggctcctgctcaagcc

SEQ ID NO: 51 - DNA, 63 bp, synthetic construct
  source 1..63: mol_type = other DNA
atgaataacctgctctgttgtgcgctcgtgttcctggacatttctataaaatggacaacgcaa

SEQ ID NO: 52 - DNA, 69 bp, synthetic construct
  source 1..69: mol_type = other DNA
atgcaaatgtctcctgcccttacctgtctcgtacttggtcttgcgctcgtatttggagagggatcagcc

SEQ ID NO: 53 - DNA, 102 bp, synthetic construct
  source 1..102: mol_type = other DNA
atggcaagggctgcactcagtgctgccccgtctaatcccagattgcttcgagttgcattgcttcttctgttgctggttgcagctggtaggagagcagcgggt

SEQ ID NO: 54 - DNA, 63 bp, synthetic construct
  source 1..63: mol_type = other DNA
atgaatgcaaaagtcgtggtcgtgctggttttggttctgacggcgttgtgtcttagtgatggg

SEQ ID NO: 55 - DNA, 66 bp, synthetic construct
  source 1..66: mol_type = other DNA
atggaacgcattgtgatctgcctgatggtcatcttcctgggcaccttagtgcacaagtcgagcagc

SEQ ID NO: 56 - DNA, 1626 bp, Homo sapiens
  source 1..1626: mol_type = other DNA
atgtgccatcagcagcttgtcatatcttggttttcacttgtattcctggccagccctttggttgcgatctgggagctcaagaaggatgtgtacgttgtagagctggactggtaccccgatgctcccggtgagatggtcgttttgacatgtgacactccagaagaggacggtattacgtggactctggaccagtcctccgaagttcttggttctggtaagactctgactatccaggtgaaagaatttggggatgcgggacaatacacatgccacaagggaggcgaggtgttgtctcatagtttgctgcttctccacaagaaagaggatggaatctggagcaccgacatactcaaggatcaaaaggaacccaaaaataagacatttctgcgatgtgaggctaagaactatagtggccgcttcacttgttggtggctgactaccatcagcacagatctcacgttttcagtaaaaagtagtagaggttcaagtgatcctcaaggggtaacgtgcggtgctgcaacactgtctgctgaacgcgtaagaggagataataaggagtacgagtattccgtagaatgccaagaggacagtgcttgtcctgcggccgaggagtctctcccaatagaagtgatggtggacgcggtgcataaactgaaatatgagaactacacaagcagtttttttataagagatatcatcaagcccgatccgccgaagaatttgcaacttaaaccgcttaaaaactcacgccaggttgaagtatcctgggagtatccggatacatggtcaacaccacacagctatttttcccttaccttctgtgtgcaggtccaagggaagagcaaaagggagaagaaggacagggtattcactgataaaacttccgcgacggtcatctgccgaaaaaacgctagtatatctgtacgggcgcaggataggtactatagttcttcttggtctgagtgggcctcagttccgtgctctgggggaggaagtggaggagggtccggcggtggaagcgggggagggagtcgcaacttgccagtggctacaccagatccaggcatgtttccatgtctgcatcattcccagaatctcctgagagcggtgtcaaatatgctccaaaaagcgagacaaacactggaattttacccgtgtaccagtgaggagattgatcacgaggacataaccaaggacaagacctcaactgtagaagcgtgtttgccgctggagttgactaagaatgagtcctgcctcaattccagagaaacttcattcattactaacggcagttgtcttgcatcccggaaaacgtcctttatgatggccctttgccttagttcaatttacgaggatcttaaaatgtatcaagtggagtttaaaaccatgaatgctaaacttcttatggaccccaaacgacaaatttttctggatcagaatatgcttgccgtgatagacgaactcatgcaggcgcttaattttaactccgaaacagttccacaaaaatctagccttgaagaacctgatttttataaaacgaagattaaactgtgtatcctgctgcatgcctttcgcatccgagctgtcacaatcgatagggttatgtcctaccttaacgcgagctag

SEQ ID NO: 57 - DNA, 1623 bp, synthetic construct
  source 1..1623: mol_type = other DNA
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagt

SEQ ID NO: 58 - DNA, 1635 bp, Mus musculus
  source 1..1635: mol_type = other DNA
atgtgtccacagaagctgacaataagttggtttgccattgtcctcctggtgagcccactcatggcaatgtgggaactcgaaaaggatgtctacgtggtagaagtagattggactccagacgcgccaggggagacagtgaatttgacatgtgacacaccagaagaagatgacattacatggacatctgaccaacgccatggcgtaatagggagtgggaaaacactcacgatcacagttaaagagttcttggatgctggtcaatatacttgccataaaggcggcgagacactcagccactcacatttgcttttgcataaaaaagagaatggcatttggagcactgaaatacttaagaactttaagaacaagacatttctcaagtgtgaggcccctaattacagcggcaggttcacgtgctcatggctggtccagcgcaacatggacctcaagtttaacataaaatcttcttcctcttcacctgactccagagctgttacttgcggcatggcttctctgagcgcagaaaaagtaacgttggatcaaagagactacgaaaagtactctgtttcttgtcaagaggatgttacgtgcccgacggccgaagaaacgcttccaattgaactcgcgttggaagctcgccaacaaaacaagtatgaaaactacagtacaagcttctttatacgggatataattaaacccgatccccccaagaacttgcaaatgaaaccacttaagaacagccaggtggaagtttcctgggagtatccagactcatggagtactcctcacagctatttttctctgaaattctttgtaaggatacaacggaagaaagagaagatgaaagagaccgaggagggttgtaatcagaagggagcgtttctcgtggagaaaacgtctaccgaagtccaatgtaaaggtggcaatgtgtgcgtccaagctcaggatagatactataattcaagttgctccaagtgggcctgtgttccatgccgcgttcggagcgggggaggtagcggaggaggtagtgggggtgggtcaggaggagggagtcgagttatcccggtgtcaggccccgcacgctgcttgagccagagtcgcaacctccttaagacaacagatgacatggtgaaaacagcacgcgaaaagcttaaacactactcttgtacggcggaggatattgatcacgaggatattacccgagaccaaactagcactttgaaaacctgtctgccccttgaacttcataaaaatgagagctgtctggctacacgagagacgtcaagtacgactaggggcagctgtctcccgccgcaaaagacaagcctcatgatgacgctctgtttgggttccatttacgaggacttgaaaatgtatcaaacggagttccaggctataaatgcggcgttgcagaaccataaccatcaacaaattatacttgataaaggcatgttggtggcgattgatgaactcatgcagagtctcaatcacaacggggaaacgttgagacagaaacccccagtcggtgaagcggacccatatcgagtaaaaatgaagctctgcattctgcttcacgcattcagcactagagttgttaccatcaaccgggtaatgggatatctctccagtgcgtag

SEQ ID NO: 59 - DNA, 465 bp, synthetic construct
  source 1..465: mol_type = other DNA
atggaacgcattgtgatctgcctgatggtcatcttcctgggcaccttagtgcacaagtcgagcagccagggacaggacaggcacatgattagaatgcgccagctcatcgatatcgtggaccagttgaagaactacgtgaacgacctggtgcccgagttcctgccggcccccgaagatgtggaaaccaattgcgaatggtcggcattttcctgctttcaaaaggcacagctcaagtccgctaacaccgggaacaacgaacggatcatcaacgtgtccatcaaaaagctgaagcggaagcctccctccaccaacgccggacggaggcagaagcataggctgacttgcccgtcatgcgactcctacgagaagaagccgccgaaggagttcctggagcggttcaagtcgctcctgcaaaagatgattcatcagcacctgtcctcccggactcatgggtctgaggattca

SEQ ID NO: 60 - DNA, 2163 bp, synthetic construct
  source 1..2163: mol_type = other DNA
atgtgccatcagcaactcgtcatctcctggttctcccttgtgttcctcgcttcccctctggtcgccatttgggaactgaagaaggacgtctacgtggtcgagctggattggtacccggacgcccctggagaaatggtcgtgctgacttgcgatacgccagaagaggacggcataacctggaccctggatcagagctccgaggtgctcggaagcggaaagaccctgaccattcaagtcaaggagttcggcgacgcgggccagtacacttgccacaagggtggcgaagtgctgtcccactccctgctgctgctgcacaagaaagaggatggaatctggtccactgacatcctcaaggaccaaaaagaaccgaagaacaagaccttcctccgctgcgaagccaagaactacagcggtcggttcacctgttggtggctgacgacaatctccaccgacctgactttctccgtgaagtcgtcacggggatcaagcgatcctcagggcgtgacctgtggagccgccactctgtccgccgagagagtcaggggagacaacaaggaatatgagtactccgtggaatgccaggaggacagcgcctgccctgccgcggaagagtccctgcctatcgaggtcatggtcgatgccgtgcataagctgaaatacgagaactacacttcctccttctttatccgcgacatcatcaagcctgacccccccaagaacttgcagctgaagccactcaagaactcccgccaagtggaagtgtcttgggaatatccagacacttggagcaccccgcactcatacttctcgctcactttctgtgtgcaagtgcagggaaagtccaaacgggagaagaaagaccgggtgttcaccgacaaaacctccgccactgtgatttgtcggaagaacgcgtcaatcagcgtccgggcgcaggatagatactactcgtcctcctggagcgaatgggccagcgtgccttgttccggtggcggatcaggcggaggttcaggaggaggctccggaggaggttcccggaacctccctgtggcaacccccgaccctggaatgttcccgtgcctacaccactcccaaaacctcctgagggctgtgtcgaacatgttgcagaaggcccgccagacccttgagttctacccctgcacctcggaagaaattgatcacgaggacatcaccaaggacaagacctcgaccgtggaagcctgcctgccgctggaactgaccaagaacgaatcgtgtctgaactcccgcgagacaagctttatcactaacggcagctgcctggcgtcgagaaagacctcattcatgatggcgctctgtctttcctcgatctacgaagatctgaagatgtatcaggtcgagttcaagaccatgaacgccaagctgctcatggacccgaagcggcagatcttcctggaccagaatatgctcgccgtgattgatgaactgatgcaggccctgaatttcaactccgagactgtgcctcaaaagtccagcctggaagaaccggacttctacaagaccaagatcaagctgtgcatcctgttgcacgctttccgcattcgagccgtgaccattgaccgcgtgatgtcctacctgaacgccagtagacggaaacgcggaagcggagagggcagaggctcgctgcttacatgcggggacgtggaagagaaccccggtccgatggaacgcattgtgatctgcctgatggtcatcttcctgggcaccttagtgcacaagtcgagcagccagggacaggacaggcacatgattagaatgcgccagctcatcgatatcgtggaccagttgaagaactacgtgaacgacctggtgcccgagttcctgccggcccccgaagatgtggaaaccaattgcgaatggtcggcattttcctgctttcaaaaggcacagctcaagtccgctaacaccgggaacaacgaacggatcatcaacgtgtccatcaaaaagctgaagcggaagcctccctccaccaacgccggacggaggcagaagcataggctgacttgcccgtcatgcgactcctacgagaagaagccgccgaaggagttcctggagcggttcaagtcgctcctgcaaaagatgattcatcagcacctgtcctcccggactcatgggtctgaggattca

SEQ ID NO: 61 - DNA, 2103 bp, Homo sapiens
  source 1..2103: mol_type = other DNA
atgtgccatcagcagcttgtcatatcttggttttcacttgtattcctggccagccctttggttgcgatctgggagctcaagaaggatgtgtacgttgtagagctggactggtaccccgatgctcccggtgagatggtcgttttgacatgtgacactccagaagaggacggtattacgtggactctggaccagtcctccgaagttcttggttctggtaagactctgactatccaggtgaaagaatttggggatgcgggacaatacacatgccacaagggaggcgaggtgttgtctcatagtttgctgcttctccacaagaaagaggatggaatctggagcaccgacatactcaaggatcaaaaggaacccaaaaataagacatttctgcgatgtgaggctaagaactatagtggccgcttcacttgttggtggctgactaccatcagcacagatctcacgttttcagtaaaaagtagtagaggttcaagtgatcctcaaggggtaacgtgcggtgctgcaacactgtctgctgaacgcgtaagaggagataataaggagtacgagtattccgtagaatgccaagaggacagtgcttgtcctgcggccgaggagtctctcccaatagaagtgatggtggacgcggtgcataaactgaaatatgagaactacacaagcagtttttttataagagatatcatcaagcccgatccgccgaagaatttgcaacttaaaccgcttaaaaactcacgccaggttgaagtatcctgggagtatccggatacatggtcaacaccacacagctatttttcccttaccttctgtgtgcaggtccaagggaagagcaaaagggagaagaaggacagggtattcactgataaaacttccgcgacggtcatctgccgaaaaaacgctagtatatctgtacgggcgcaggataggtactatagttcttcttggtctgagtgggcctcagttccgtgctctgggggaggaagtggaggagggtccggcggtggaagcgggggagggagtcgcaacttgccagtggctacaccagatccaggcatgtttccatgtctgcatcattcccagaatctcctgagagcggtgtcaaatatgctccaaaaagcgagacaaacactggaattttacccgtgtaccagtgaggagattgatcacgaggacataaccaaggacaagacctcaactgtagaagcgtgtttgccgctggagttgactaagaatgagtcctgcctcaattccagagaaacttcattcattactaacggcagttgtcttgcatcccggaaaacgtcctttatgatggccctttgccttagttcaatttacgaggatcttaaaatgtatcaagtggagtttaaaaccatgaatgctaaacttcttatggaccccaaacgacaaatttttctggatcagaatatgcttgccgtgatagacgaactcatgcaggcgcttaattttaactccgaaacagttccacaaaaatctagccttgaagaacctgatttttataaaacgaagattaaactgtgtatcctgctgcatgcctttcgcatccgagctgtcacaatcgatagggttatgtcctaccttaacgcgagccggcgcaagaggggttccggagagggaaggggtagtctgctcacctgcggcgatgttgaagaaaatcctggtcccatggcgcaaagtctggctctttcactcctgatcctggtcttggccttcgggattccgaggacccaaggaagtgatggtggcgcccaagattgttgccttaaatacagccagcggaaaatacccgcgaaagtggtcaggagttatagaaaacaggagccttccctgggttgtagtatccccgccatacttttcctcccgagaaaacggagccaggccgaactgtgcgctgaccctaaggaactttgggtgcaacaacttatgcaacacctggataagacaccttctcctcaaaagccagctcagggctgccgaaaagatagaggcgcctcaaaaaccggaaaaaagggcaaaggttctaaaggatgtaagcggactgaacgctctcaaacgcctaaagggccgtag

SEQ ID NO: 62 - DNA, 2109 bp, Mus musculus
  source 1..2109: mol_type = other DNA
atgtgtccacagaagctgacaataagttggtttgccattgtcctcctggtgagcccactcatggcaatgtgggaactcgaaaaggatgtctacgtggtagaagtagattggactccagacgcgccaggggagacagtgaatttgacatgtgacacaccagaagaagatgacattacatggacatctgaccaacgccatggcgtaatagggagtgggaaaacactcacgatcacagttaaagagttcttggatgctggtcaatatacttgccataaaggcggcgagacactcagccactcacatttgcttttgcataaaaaagagaatggcatttggagcactgaaatacttaagaactttaagaacaagacatttctcaagtgtgaggcccctaattacagcggcaggttcacgtgctcatggctggtccagcgcaacatggacctcaagtttaacataaaatcttcttcctcttcacctgactccagagctgttacttgcggcatggcttctctgagcgcagaaaaagtaacgttggatcaaagagactacgaaaagtactctgtttcttgtcaagaggatgttacgtgcccgacggccgaagaaacgcttccaattgaactcgcgttggaagctcgccaacaaaacaagtatgaaaactacagtacaagcttctttatacgggatataattaaacccgatccccccaagaacttgcaaatgaaaccacttaagaacagccaggtggaagtttcctgggagtatccagactcatggagtactcctcacagctatttttctctgaaattctttgtaaggatacaacggaagaaagagaagatgaaagagaccgaggagggttgtaatcagaagggagcgtttctcgtggagaaaacgtctaccgaagtccaatgtaaaggtggcaatgtgtgcgtccaagctcaggatagatactataattcaagttgctccaagtgggcctgtgttccatgccgcgttcggagcgggggaggtagcggaggaggtagtgggggtgggtcaggaggagggagtcgagttatcccggtgtcaggccccgcacgctgcttgagccagagtcgcaacctccttaagacaacagatgacatggtgaaaacagcacgcgaaaagcttaaacactactcttgtacggcggaggatattgatcacgaggatattacccgagaccaaactagcactttgaaaacctgtctgccccttgaacttcataaaaatgagagctgtctggctacacgagagacgtcaagtacgactaggggcagctgtctcccgccgcaaaagacaagcctcatgatgacgctctgtttgggttccatttacgaggacttgaaaatgtatcaaacggagttccaggctataaatgcggcgttgcagaaccataaccatcaacaaattatacttgataaaggcatgttggtggcgattgatgaactcatgcagagtctcaatcacaacggggaaacgttgagacagaaacccccagtcggtgaagcggacccatatcgagtaaaaatgaagctctgcattctgcttcacgcattcagcactagagttgttaccatcaaccgggtaatgggatatctctccagtgcgcggcgcaagaggggttccggagagggaaggggtagtctgctcacctgcggcgatgttgaagaaaatcctggtcccatggcgcaaatgatgaccctttccctgctgagtcttgtcctcgcgctctgcatcccgtggacgcaggggtctgatggggggggccaagactgttgcctgaagtattcacaaaaaaagataccgtactctattgtcagagggtacaggaagcaagaaccctccttgggttgccctataccagcaattcttttctccccacgcaagcattccaaaccagaactgtgtgcgaaccccgaggagggttgggtacagaacttgatgcgaaggcttgaccagcccccagcccctggcaagcagtcacctgggtgcagaaaaaacagaggtacttcaaagagcggcaagaaaggcaaagggagtaaaggatgtaaaagaacggagcagacccagccttcacgaggctag

SEQ ID NO: 63 - skipped

SEQ ID NO: 64 - DNA, 465 bp, Mus musculus
  source 1..465: mol_type = other DNA
atgtttcatgtgtccttcaggtacatatttggtatcccaccacttatattggtgctcttgcctgtaaccagctctgaatgtcatataaaagacaaggagggcaaagcatacgagtccgtattgatgatctcaatcgatgaacttgacaagatgacagggaccgattctaattgtccaaataacgagccaaacttctttcggaaacacgtgtgtgatgatacaaaagaagctgcttttcttaacagagctgccagaaaactcaagcagttcctcaagatgaatatatccgaggaatttaacgtgcatctcctcacagtatctcagggaactcaaacccttgtaaactgcacttctaaggaggagaagaatgtcaaagagcagaagaaaaatgatgcatgttttttgaaacggctgttgagggagatcaaaacatgctggaataaaatcctcaagggctcaatttag

SEQ ID NO: 65 - DNA, 1428 bp, Homo sapiens
  source 1..1428: mol_type = other DNA
atggaaacagacacattgctgctttgggtattgttgctctgggtgcctggatcaacaggaaactgggtaaacgtaatttcagatctgaagaagatcgaggaccttattcaatccatgcacatcgatgccactctctacaccgaaagcgacgttcacccatcttgcaaggtgaccgctatgaaatgtgaattgttggaacttcaggtaatttctctggagagcggcgatgcctcaatacatgacaccgttgaaaatcttatcatccttgctaatgattcactctctagtaatgggaacgtaacagagagcgggtgtaaggagtgtgaagaactggaggagaaaaacattaaggaatttttgcagtcattcgtccatatagtgcaaatgttcataaacacttccagaagaaagcgaggctctggggaggggcgaggctctctgctgacctgtggggatgtagaagagaatccaggtcccatggaccggctgaccagctcattcctgcttctgattgtgccagcctacgtgctctccatcacatgtcctcccccaatgagcgtcgagcatgctgacatctgggtgaagtcatactccttgtacagcagagagagatacatttgtaattccggattcaagcgcaaggccggcacctcctctctgacagagtgcgtccttaacaaagcaaccaacgtagcacattggaccacaccatccttgaagtgcatacgagaacctaaatcttgcgataagactcatacttgtccaccttgtccagccccagaactgcttggcggaccctcagtatttttgttcccaccaaagccaaaagacacactcatgatatccagaactcctgaggtgacctgtgtcgttgtagacgtttcccacgaagatcctgaagtaaaattcaactggtacgtggatggggtcgaagtccataacgccaagactaaaccaagggaggaacagtataactctacttaccgagtagtttctgtgttgaccgtgctgcaccaggactggttgaacgggaaggagtacaaatgcaaggtgagcaataaagctctgcccgcaccaatcgaaaagacaatatctaaggccaaggggcagccacgagagccccaggtatacacactgccaccctcacgcgatgaattgactaagaaccaggtttccctgacctgtcttgtaaaaggtttctacccttccgacatagctgttgagtgggaaagtaacgggcagccagagaacaattacaagacaactccacccgttcttgatagcgatggatcattttttctgtattccaaactcactgtcgataaaagtcgctggcagcaaggcaatgtttttagctgctcagtcatgcacgaagcactgcataatcactacacacaaaaaagtttgtcccttagccctggtaagtag

SEQ ID NO: 66 - DNA, 1080 bp, Homo sapiens
  source 1..1080: mol_type = other DNA
atgtactcaatgcagttggcctcctgtgtaacattgaccttggtcctcttggtcaacagcaattggatcgatgtacgctacgacttggagaagattgagtcccttatacagagtatacacatagatacaaccttgtatactgacagtgacttccatcccagctgtaaagtgactgcaatgaactgttttttgttggagttgcaagtaattctgcatgaatacagcaacatgaccctcaatgaaaccgttaggaatgtcctttatctcgcaaattctactctgagtagcaataagaatgttgccgaaagcggctgcaaggagtgcgaagaactggaggaaaaaactttcaccgagtttctccagagtttcatcagaattgtccaaatgttcattaatacaagtagtggtggtgggagcgggggtggaggcagtgggggaggtgggagcggaggtggagggtccggaggggggagccttcaaggcactacttgtcctccacccgtatccatcgagcacgccgatattcgagttaaaaattatagtgttaatagcagagaacgatacgtctgcaactcagggtttaagagaaaggccggaacttcaactctcatagaatgcgtgattaataagaatactaacgtcgcacattggactactcccagtctcaagtgcatacgcgatccatctctcgctcattactcaccagtacctacagtggttactcctaaggtgacctctcagcccgaatcaccatctcccagcgcaaaagagcctgaggccttttctcctaaatcagacactgctatgactacagaaacagccataatgccaggaagccggctgacaccatctcaaactaccagcgcaggcacaactgggactggctcccacaaaagctcacgcgcaccaagtctcgccgcaacaatgacattggagcctacagccagcacatctcttagaatcacagaaatttctccccacagtagcaagatgaccaaggtggcaattagtaccagcgtccttcttgtaggagctggagttgtgatggcatttttggcatggtatatcaaaagcaggtag

SEQ ID NO: 67 - DNA, 489 bp, Mus musculus
  source 1..489: mol_type = other DNA
atgaagatcctcaagccatacatgcgaaacactagtattagctgttacttgtgttttctgctgaatagtcattttttgactgaagcaggaatccatgtatttatactcggttgtgtgtctgtaggtctgccaaagactgaggctaattggattgacgtgcgctatgatcttgaaaaaatagagtccttgattcaatcaatacacatcgataccactctctacaccgacagtgatttccatccttcctgcaaggtaacagctatgaattgcttcctcctggagctccaagtcattctccatgagtactccaacatgactttgaacgaaactgtaagaaacgtattgtatctggctaatagcaccttgtctagtaacaaaaatgtggcagagagcggctgcaaagaatgtgaagaattggaagagaaaacatttacagagttcctgcaatcctttattcgcatcgtccaaatgtttatcaatacctcttag

SEQ ID NO: 68 - DNA, 438 bp, Mus musculus
  source 1..438: mol_type = other DNA
atgtattccatgcaacttgccagttgtgtaacccttactctcgtcctgctcgttaattccgctggtgctaactggatagatgttcgatacgatctggaaaagattgagtcccttatccaatccattcatatagataccaccctttatactgacagcgacttccatccttcttgcaaggtgaccgctatgaattgtttcctgctggaactccaagttattctgcatgaatactctaatatgacacttaacgagaccgtaagaaatgttctctatctcgctaatagtactttgagctcaaataagaacgtggccgagtctgggtgtaaggaatgcgaagagctggaagaaaagacattcaccgagtttctccagtctttcatacggattgtgcagatgtttatcaacacatcagattacaaagacgacgatgataagtag

SEQ ID NO: 69 - DNA, 579 bp, Mus musculus
  source 1..579: mol_type = other DNA
atggcagccatgtctgaggactcttgtgtgaactttaaagaaatgatgttcatagacaatacactctactttatacctgaggagaatggagatttggaatctgacaactttggcaggctgcattgcactaccgcagttatccgaaacatcaacgatcaggtactgtttgttgataaaagacaacctgtattcgaggacatgaccgacatagatcagtctgcctcagagccccagactaggcttatcatctatatgtacaaggacagcgaagtacgaggcctggctgttacactctcagtcaaagactctaagatgagcaccctgtcatgcaagaacaaaattatcagttttgaggagatggacccacctgaaaacatagatgacattcagtcagacctcattttttttcaaaagcgggtaccaggacacaacaaaatggaatttgaatcatcactctacgaaggacatttccttgcatgccagaaagaggatgacgcattcaaattgatcctgaaaaaaaaggacgaaaatggtgataaatcagtcatgtttacattgaccaatcttcaccaaagttag

SEQ ID NO: 70 - DNA, 579 bp, Mus musculus
  source 1..579: mol_type = other DNA
atggctgcaatgtctgaagatagctgtgtcaactttaaggagatgatgttcattgataatactttgtactttatacctgaagaaaatggagaccttgagtcagacaacttcgggagactgcactgcacaactgccgttatccgaaacataaatgatcaagtattgttcgtggacaaaagacaaccagtctttgaggatatgacagacatcgaccaatccgcatctgaacctcagactaggctgatcatctatatgtacgccgactccgaagtaagaggccttgctgtgacacttagtgttaaggatagtaagatgagcacactgtcctgtaagaataagattatatcttttgaagagatggaccctcccgagaacatagatgacatccagagcgacttgatcttctttcagaagcgagtgccaggccataacaagatggaatttgaatcatctctttatgaaggccatttcctcgcatgtcaaaaggaggacgatgccttcaagctcattctgaaaaaaaaagacgagaacggtgataagagcgtgatgttcactctgacaaatctgcaccagtcatag

SEQ ID NO: 71 - DNA, 534 bp, Homo sapiens
  source 1..534: mol_type = other DNA
atgtatcgcatgcaactcctgtcctgcattgctctgagcttggctttggtaaccaactcatacttcgggaaactggagagtaaactctccgtaatcaggaatcttaatgaccaagtattgtttattgaccagggcaaccgcccgttgttcgaggatatgactgattctgactgtcgggataacgctccgagaactatctttatcatttcaatgtacaaggacagccaaccgcggggtatggctgtgacaatcagtgtcaaatgtgagaagatttccacgctgtcctgcgaaaacaagataatttctttcaaagaaatgaacccccctgacaatataaaggatacaaagagtgatatcatcttctttcagaggtccgtgcccggccacgataataagatgcaatttgaaagttcatcttatgaggggtactttttggcatgcgagaaagaaagggatctcttcaagttgatcctgaagaaggaggacgaattgggcgaccgctccatcatgttcacagtccagaacgaggactag

SEQ ID NO: 72 - DNA, 534 bp, Homo sapiens
  source 1..534: mol_type = other DNA
atgtaccgcatgcagctcctgagttgtattgccctttccctcgctctcgttaccaattcttacttcggtaagcttgcctctaaactctctgttattaggaacttgaacgaccaagtccttttcatagaccaagggaacagaccactgtttgaagatatgacggatagcgattgccgagataatgcccctaggacgatttttatcattagtatgtatgcggactctcaaccgagggggatggccgttactataagtgtgaaatgcgagaaaatatcaacgctcagttgtgagaacaaaatcataagtttcaaggagatgaatccacctgataacatcaaagacactaagtctgatattatatttttccaacgaagtgttccgggacacgataacaaaatgcaatttgagagctcctcatacgagggctacttcctcgcgtgtgagaaagaaagggatttgtttaagcttatcctcaagaaagaggacgagttgggggatcggagcataatgtttaccgtacagaatgaggactag

SEQ ID NO: 73 - DNA, 441 bp, Mus musculus
  source 1..441: mol_type = other DNA
atggagcggacactcgtgtgtcttgtcgtaatttttctcgggacagtcgcacacaagtcctcaccccagggtcctgatcgccttctcatacgcctccgacatttgatcgacattgtagagcagctcaaaatttacgagaatgacctcgatcccgagcttttgagtgctccccaagacgttaagggtcattgcgagcacgcagcttttgcttgcttccagaaggccaagttgaaaccaagcaaccctggtaataataagactttcatcatcgacttggtcgcccaactccgaaggaggctgcctgcccggcgcggaggaaaaaaacaaaagcatattgcaaagtgtccttcatgtgattcatacgaaaagcggactcccaaagagttcttggaaaggttgaaatggcttcttcagaagatgattcatcaacatttgtcatag

SEQ ID NO: 74 - DNA, 564 bp, Homo sapiens
  source 1..564: mol_type = other DNA
atgaccaacaaatgccttttgcaaattgccctgcttttgtgttttagcactaccgcattgagcatgtcatataacctcctcggcttccttcagagatcatcaaactttcagtgtcagaaactgctttggcaacttaatggcaggctcgaatattgtctgaaagatcggatgaatttcgacattccagaagaaataaaacagcttcaacaattccagaaagaggacgccgccctgactatttacgagatgctccagaatatcttcgccattttccggcaggacagctcatccacggggtggaatgagactattgtagaaaatcttctggctaatgtgtaccatcaaattaatcacctcaaaacggtgcttgaggaaaaacttgaaaaggaagatttcacacggggcaagttgatgtcctccctgcaccttaaacgatactacggcaggattcttcattacttgaaggctaaggagtatagccattgcgcgtggacaattgtacgggtagaaatactgcgaaacttttatttcatcaaccggctcactggataccttagaaattag

SEQ ID NO: 75 - DNA, 549 bp, Mus musculus
  source 1..549: mol_type = other DNA
atgaacaatcggtggatactccacgccgcatttctcctctgctttagcacgacggccctgtccatcaactacaaacagcttcagttgcaggagcggactaacataaggaagtgccaggaactgctggaacagcttaatggtaaaattaatcttacataccgagctgacttcaaaattcctatggaaatgaccgagaagatgcagaaatcctacacggcattcgccatccaggaaatgctccagaacgtatttctcgtgttccgcaataatttctcttctacgggttggaacgaaaccattgttgttagactgcttgacgaactgcatcagcaaaccgtgttccttaaaaccgtgcttgaggagaagcaggaggagcgcctgacttgggagatgtctagtaccgcacttcacttgaaatcctactactggcgcgttcagcggtatctgaagctgatgaagtataactcatacgcctggatggtagtgcgcgcagagatcttcagaaactttcttatcatccggcgactgacccgaaactttcagaattag

SEQ ID NO: 76 - DNA, 501 bp, Homo sapiens
  source 1..501: mol_type = other DNA
atgaagtacactagctatatattggccttccagctttgcatcgtattgggtagcctcggatgctattgccaagacccgtatgtcaaagaagccgaaaatctcaaaaagtatttcaatgccggacactcagacgtcgcggataacggtacactgtttcttggcatcctgaaaaattggaaggaagagagtgacagaaaaataatgcagtcacaaatagtgtccttttactttaagctgttcaaaaatttcaaggatgaccaaagtatccagaagagtgttgaaactatcaaagaggacatgaatgtgaaattctttaacagtaataagaagaagcgcgatgacttcgagaaactcactaattacagcgtaacggatcttaacgtccaacgcaaggcaatccacgagcttatacaggtaatggctgagcttagtcccgcagccaagacagggaagagaaaaaggtctcaaatgctttttcggggccggcgagcttcacaatag

SEQ ID NO: 77 - DNA, 468 bp, Mus musculus
  source 1..468: mol_type = other DNA
atgaacgctacgcattgcatcctcgcactccaattgttcctcatggctgtgtcagggtgttactgtcacggtactgtcatagaaagcctcgaatccctgaataactattttaacagtagcggtatagatgtagaagaaaagtctctctttcttgacatctggaggaattggcaaaaggatggagacatgaagattctccaatctcagattatatcattttacttgaggctttttgaggttctgaaggataaccaggcgatcagcaataatatcagcgtaattgaatctcaccttattacaacatttttctcaaattccaaggcaaagaaagatgctttcatgtctatcgcgaaatttgaggtgaacaatcctcaggtacaaaggcaagcctttaacgagctgattagagttgtacatcagttgttgcccgaaagtagtcttagaaaacgcaaacggagccgatgctag

SEQ ID NO: 78 - DNA, 570 bp, Mus musculus
  source 1..570: mol_type = other DNA
atggcaaggttgtgcgcttttctcatggtactggctgtgctctcctattggcctacttgttctctgggatgcgacttgccacagacccacaatctgcggaataagagggctctgactctgctggtgcaaatgagacggctctctccacttagctgtttgaaagatagaaaggatttcgggttcccccaggagaaggtggatgcccagcagatcaagaaggcacaggctatccccgtcctttccgagctgacccagcaaattttgaacatctttacaagtaaggatagttcagctgcatggaataccacacttttggattctttttgtaacgatctgcatcagcagctgaacgatctccagggatgcctgatgcagcaagtcggcgtgcaagaatttccactcacccaggaggacgctctgctcgcagtgcgaaagtattttcaccgaattaccgtgtacctccgggagaaaaagcattcaccctgcgcttgggaagtagtcagggccgaagtatggagagcccttagtagctccgctaatgtactgggccggttgcgggaagagaaatag

SEQ ID NO: 79-111 - skipped (33 entries)

SEQ ID NO: 112 - protein, 22 aa, Homo sapiens
MCHQQLVISWFSLVFLASPLVA

SEQ ID NO: 113 - protein, 14 aa, synthetic construct
MQLLSCIALILALV

SEQ ID NO: 114 - protein, 20 aa, Homo sapiens
MYRMQLLSCIALSLALVTNS

SEQ ID NO: 115 - protein, 15 aa, Homo sapiens
MNLLLILTFVAAAVA

SEQ ID NO: 116 - protein, 17 aa, synthetic construct
MGVKVLFALICIAVAEA

SEQ ID NO: 117 - protein, 24 aa, Homo sapiens
MPMGSLQPLATLYLLGMLVASCLG

SEQ ID NO: 118 - protein, 21 aa, Mus musculus
METDTLLLWVLLLWVPGSTGD

SEQ ID NO: 119 - protein, 22 aa, Homo sapiens
MDMRVPAQLLGLLLLWLRGARC

SEQ ID NO: 120 - protein, 16 aa, synthetic construct
MKCLLYLAFLFIGVNC

SEQ ID NO: 121 - protein, 28 aa, Homo sapiens
MNIKGSPWKGSLLLLLVSNLLLCQSVAP

SEQ ID NO: 122 - protein, 18 aa, Homo sapiens
MKWVTFISLLFLFSSAYS

SEQ ID NO: 123 - protein, 19 aa, Homo sapiens
MTRLTVLALLAGLLASSRA

SEQ ID NO: 124 - protein, 17 aa, Homo sapiens
MRAWIFFLLCLAGRALA

SEQ ID NO: 125 - protein, 16 aa, Homo sapiens
MPLLLLLPLLWAGALA

SEQ ID NO: 126 - protein, 29 aa, Homo sapiens
MNSFSTSAFGPVAFSLGLLLVLPAAFPAP

SEQ ID NO: 127 - protein, 20 aa, Homo sapiens
MTSKLAVALLAAFLISAALC

SEQ ID NO: 128 - protein, 23 aa, Homo sapiens
MKVSAALLCLLLIAATFIPQGLA

SEQ ID NO: 129 - protein, 26 aa, Homo sapiens
MGAAARTLRLALGLLLLATLLRPADA

SEQ ID NO: 130 - protein, 21 aa, Homo sapiens
MSPLLRRLLLAALLQLAPAQA

SEQ ID NO: 131 - protein, 21 aa, Homo sapiens
MNNLLCCALVFLDISIKWTTQ

SEQ ID NO: 132 - protein, 23 aa, Homo sapiens
MQMSPALTCLVLGLALVFGEGSA

SEQ ID NO: 133 - protein, 34 aa, Homo sapiens
MARAALSAAPSNPRLLRVALLLLLLVAAGRRAAG

SEQ ID NO: 134 - protein, 21 aa, Homo sapiens
MNAKVVVVLVLVLTALCLSDG

SEQ ID NO: 135 - protein, 22 aa, synthetic construct
MERIVICLMVIFLGTLVHKSSS

SEQ ID NO: 136 - protein, 21 aa, synthetic construct
MALPVTALLLPLALLLHAARP

SEQ ID NO: 137 - protein, 21 aa, synthetic construct
MALPVTALLLPLALLLHAARP

SEQ ID NO: 138 - protein, 21 aa, synthetic construct
MLLVTSLLLCELPHPAFLLIP

SEQ ID NO: 139 - DNA, 63 bp, synthetic construct
  source 1..63: mol_type = other DNA
atggccttaccagtgaccgccttgctcctgccgctggccttgctgctccacgccgccaggccg

SEQ ID NO: 140 - DNA, 63 bp, synthetic construct
  source 1..63: mol_type = other DNA
atggcgctcccggtgacagcacttctcttgcctcttgccctgctgttgcatgccgcgcgccca

SEQ ID NO: 141 - DNA, 63 bp, synthetic construct
  source 1..63: mol_type = other DNA
atgttgctcgtgacatccctcttgctttgtgagttgcctcatcccgcattcctgctcatccca

SEQ ID NO: 142 - protein, 10 aa, synthetic construct
DEMEECSQHL

SEQ ID NO: 143 - protein, 9 aa, synthetic construct
EDVVPCSMG

SEQ ID NO: 144 - skipped

SEQ ID NO: 145 - protein, 19 aa, synthetic construct
EDANSEPLFAERKDABCYL

SEQ ID NO: 146 - protein, 4 aa, synthetic construct
YVAD

SEQ ID NO: 147 - protein, 5 aa, synthetic construct
VDVAD

SEQ ID NO: 148 - protein, 4 aa, synthetic construct
DEVD

SEQ ID NO: 149 - protein, 4 aa, synthetic construct
VEHD

SEQ ID NO: 150 - protein, 4 aa, synthetic construct
LGHD

SEQ ID NO: 151 - protein, 5 aa, synthetic construct
LQTDG

SEQ ID NO: 152 - protein, 8 aa, synthetic construct
EVNLDAEF

SEQ ID NO: 153 - protein, 7 aa, synthetic construct
PQGIAGQ

SEQ ID NO: 154 - protein, 7 aa, synthetic construct
ENLYFQS

SEQ ID NO: 155 - protein, 6 aa, synthetic construct
  MOD_RES 6: note = Lysine(2,4-dinitrophenyl)
HPFHLX

SEQ ID NO: 156-169 - skipped (14 entries)

SEQ ID NO: 170 - DNA, 65 bp, synthetic construct
  source 1..65: mol_type = other DNA
taggcgtgtacggtgggaggcctatataagcagagctcgtttagtgaaccgtcagatcgcctgga

SEQ ID NO: 171 - DNA, 25 bp, synthetic construct
  source 1..25: mol_type = other DNA
tctagagggtatataatgggggcca

SEQ ID NO: 172 - DNA, 63 bp, synthetic construct
  source 1..63: mol_type = other DNA
ttcgcatattaaggtgacgcgtgtggcctcgaacaccgagcgaccctgcagcgacccgcttaa

SEQ ID NO: 173 - DNA, 659 bp, synthetic construct
  source 1..659: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 174 - DNA, 251 bp, synthetic construct
  source 1..251: mol_type = other DNA
tgtttgctgcttgcaatgtttgcccattttagggtggacacaggacgctgtggtttctgagccagggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 175 - protein, 60 aa, synthetic construct
FNVLMVHKRSHTGERPLQCEICGFTCRQKGNLLRHIKLHTGEKPFKCHLCNYACQRRDAL

SEQ ID NO: 176 - protein, 4 aa, synthetic construct
PRAE

SEQ ID NO: 177 - skipped

SEQ ID NO: 178 - protein, 9 aa, synthetic construct
  VARIANT 5: note = X can be A, Y, P, S, or F
  VARIANT 6: note = X can be V, L, S, I, Y, T, or A
PRAEXXKGG

SEQ ID NO: 179 - protein, 9 aa, synthetic construct
PRAEAVKGG

SEQ ID NO: 180 - protein, 9 aa, synthetic construct
PRAEALKGG

SEQ ID NO: 181 - protein, 9 aa, synthetic construct
PRAEYSKGG

SEQ ID NO: 182 - protein, 9 aa, synthetic construct
PRAEPIKGG

SEQ ID NO: 183 - protein, 9 aa, synthetic construct
PRAEAYKGG

SEQ ID NO: 184 - protein, 9 aa, synthetic construct
PRAESSKGG

SEQ ID NO: 185 - protein, 9 aa, synthetic construct
PRAEFTKGG

SEQ ID NO: 186 - protein, 9 aa, synthetic construct
PRAEAAKGG

SEQ ID NO: 187 - protein, 9 aa, synthetic construct
DEPHYSQRR

SEQ ID NO: 188 - protein, 10 aa, synthetic construct
PPLGPIFNPG

SEQ ID NO: 189 - protein, 9 aa, synthetic construct
PLAQAYRSS

SEQ ID NO: 190 - protein, 10 aa, synthetic construct
TPIDSSFNPD

SEQ ID NO: 191 - protein, 10 aa, synthetic construct
VTPEPIFSLI

SEQ ID NO: 192 - protein, 21 aa, synthetic construct
PFFFCCFIAVAMGIRFIIMVA

SEQ ID NO: 193 - DNA, 63 bp, synthetic construct
  source 1..63: mol_type = other DNA
cccttcttcttctgttgctttatcgccgtggccatgggcatccgcttcatcattatggtggcc

SEQ ID NO: 194 - protein, 12 aa, synthetic construct
SGGGGSGGGGSG

SEQ ID NO: 195 - DNA, 795 bp, synthetic construct
  source 1..795: mol_type = other DNA
gaggatgtcgtgtgctgccacagcatctacggcaagaagaagggcgacatcgacacctaccggtacatcggcagctctggcacaggctgtgtggtcatcgtgggcagaatcgtgctgtctggcagcggaacaagcgcccctatcacagcctatgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggccccgtgatccagatgtacaccaacgtggaccaggacctcgttggctggcctgctcctcaaggcagcagaagcctgacaccttgcacctgtggctccagcgatctgtacctggtcaccagacacgccgacgtgatccctgtcagaagaagaggggattccagaggcagcctgctgagccctagacctatcagctacctgaagggctctagcggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcac

SEQ ID NO: 196 - protein, 45 aa, synthetic construct
TTTPAPRPPTPAPTIASQPLSLRPEACRPAAGGAVHTRGLDFACD

SEQ ID NO: 197 - protein, 15 aa, synthetic construct
GGGSGGGGSGGGSLQ

SEQ ID NO: 198 - protein, 14 aa, synthetic construct
ITQGLAVSTISSFF

SEQ ID NO: 199 - protein, 5 aa, synthetic construct
KNAMN

SEQ ID NO: 200 - protein, 19 aa, synthetic construct
RIRNKTNNYATYYADSVKA

SEQ ID NO: 201 - protein, 6 aa, synthetic construct
GNSFAY

SEQ ID NO: 202 - protein, 17 aa, synthetic construct
KSSQSLLYSSNQKNYLA

SEQ ID NO: 203 - protein, 7 aa, synthetic construct
WASSRES

SEQ ID NO: 204 - protein, 9 aa, synthetic construct
QQYYNYPLT

SEQ ID NO: 205 - protein, 117 aa, synthetic construct
EVQLVETGGGMVQPEGSLKLSCAASGFTFNKNAMNWVRQAPGKGLEWVARIRNKTNNYATYYADSVKARFTISRDDSQSMLYLQMNNLKIEDTAMYYCVAGNSFAYWGQGTLVTVSA

SEQ ID NO: 206 - protein, 117 aa, synthetic construct
EVQLVESGGGLVQPGGSLRLSCAASGFTFNKNAMNWVRQAPGKGLEWVGRIRNKTNNYATYYADSVKARFTISRDDSKNSLYLQMNSLKTEDTAVYYCVAGNSFAYWGQGTLVTVSA

SEQ ID NO: 207 - protein, 113 aa, synthetic construct
DIVMSQSPSSLVVSIGEKVTMTCKSSQSLLYSSNQKNYLAWYQQKPGQSPKLLIYWASSRESGVPDRFTGSGSGTDFTLTISSVKAEDLAVYYCQQYYNYPLTFGAGTKLELK

SEQ ID NO: 208 - protein, 113 aa, synthetic construct
DIVMTQSPDSLAVSLGERATINCKSSQSLLYSSNQKNYLAWYQQKPGQPPKLLIYWASSRESGVPDRFSGSGSGTDFTLTISSLQAEDVAVYYCQQYYNYPLTFGQGTKLEIK

SEQ ID NO: 209 - skipped

SEQ ID NO: 210 - RNA, 21 nt, synthetic construct
  source 1..21: mol_type = other RNA
atttatttatttatttattta

SEQ ID NO: 211 - RNA, 19 nt, synthetic construct
  source 1..19: mol_type = other RNA
ctgtttaatatttaaacag

SEQ ID NO: 212 - RNA, 51 nt, synthetic construct
  source 1..51: mol_type = other RNA
atttatttatttatttatttaacatcggttccctgtttaatatttaaacag

SEQ ID NO: 213 - RNA, 112 nt, synthetic construct
  source 1..112: mol_type = other RNA
atttatttatttatttatttaacatcggttccctgtttaatatttaaacagtgcggtaagcatttatttatttatttatttaacatcggttccctgtttaatatttaaacag

SEQ ID NO: 214 - DNA, 54 bp, synthetic construct
  source 1..54: mol_type = other DNA
atggactggacctggatcctgtttctggtggccgctgccacaagagtgcacagc

SEQ ID NO: 215 - protein, 27 aa, synthetic construct
SGGGGSGGGGSGGGGSGGGGSGGGSLQ

SEQ ID NO: 216 - protein, 22 aa, synthetic construct
MLLLVTSLLLCELPHPAFLLIP

SEQ ID NO: 217 - DNA, 66 bp, synthetic construct
  source 1..66: mol_type = other DNA
atgctgctgctggtcacatctctgctgctgtgcgagctgccccatcctgcctttctgctgatccct

SEQ ID NO: 218 - protein, 18 aa, synthetic construct
MDWTWILFLVAAATRVHS

SEQ ID NO: 219 - protein, 46 aa, synthetic construct
LLPSWAITLISVNGIFVICCLTYCFAPRCRERRRNERLRRESVRPV

SEQ ID NO: 220 - DNA, 138 bp, synthetic construct
  source 1..138: mol_type = other DNA
ctgctgccaagctgggccatcacactgatctccgtgaacggcatcttcgtgatctgttgcctgacctactgcttcgcccctcggtgcagagagcggagaagaaacgaacggctgcggagagaatctgtgcggcctgtg

SEQ ID NO: 221 - DNA, 339 bp, synthetic construct
  source 1..339: mol_type = other DNA
gacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtactactgccagcagtactacaactaccctctgaccttcggccagggcaccaagctggaaatcaaa

SEQ ID NO: 222 - DNA, 351 bp, synthetic construct
  source 1..351: mol_type = other DNA
gaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccaggttcaccatctccagagatgacagcaagaacagcctgtacctgcagatgaactccctgaaaaccgaggacaccgccgtgtactattgcgtggccggcaatagctttgcctactggggacagggcaccctggttacagtttctgct

SEQ ID NO: 223 - protein, 15 aa, synthetic construct
GGGGSGGGGSGGGGS

SEQ ID NO: 224 - DNA, 45 bp, synthetic construct
  source 1..45: mol_type = other DNA
ggcggcggaggatctggcggaggtggaagtggcggaggcggatct

SEQ ID NO: 225 - skipped

SEQ ID NO: 226 - protein, 45 aa, synthetic construct
TTTPAPRPPTPAPTIALQPLSLRPEACRPAAGGAVHTRGLDFACD

SEQ ID NO: 227 - DNA, 135 bp, synthetic construct
  source 1..135: mol_type = other DNA
acaacaacccctgctcctagacctcctacaccagctcctacaatcgccctgcagcctctgtctctgaggccagaagcttgtagaccagctgctggcggagccgtgcatacaagaggactggacttcgcctgtgat

SEQ ID NO: 228 - protein, 67 aa, synthetic construct
GALSNSIMYFSHFVPVFLPAKPTTTPAPRPPTPAPTIASQPLSLRPEACRPAAGGAVHTRGLDFACD

SEQ ID NO: 229 - DNA, 201 bp, synthetic construct
  source 1..201: mol_type = other DNA
ggcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgcaggcggagccgtgcatacaagaggactggatttcgcctgcgac

SEQ ID NO: 230 - protein, 23 aa, synthetic construct
VAISTSTVLLCGLSAVSLLACYL

SEQ ID NO: 231 - DNA, 69 bp, synthetic construct
  source 1..69: mol_type = other DNA
gtggccatcagcacaagcaccgtgctgctgtgtggactgtctgccgtttctctgctggcctgctacctg

SEQ ID NO: 232 - protein, 27 aa, synthetic construct
FWVLVVVGGVLACYSLLVTVAFIIFWV

SEQ ID NO: 233 - DNA, 81 bp, synthetic construct
  source 1..81: mol_type = other DNA
ttctgggtgctcgtggttgttggcggagtgctggcctgttactctctgctggtcaccgtggccttcatcatcttttgggtc

SEQ ID NO: 234 - protein, 20 aa, synthetic construct
VAAILGLGLVLGLLGPLAIL

SEQ ID NO: 235 - DNA, 62 bp, synthetic construct
  source 1..62: mol_type = other DNA
gtggccgccattctcggactgggacttgttctgggactgctgggacctctggccattctgct

SEQ ID NO: 236 - protein, 21 aa, synthetic construct
IYIWAPLAGTCGVLLLSLVIT

SEQ ID NO: 237 - DNA, 63 bp, synthetic construct
  source 1..63: mol_type = other DNA
atctacatctgggcccctctggctggaacatgcggagtgttgctgctgagcctggtcatcacc

SEQ ID NO: 238 - protein, 21 aa, synthetic construct
MGLAFLVLVALVWFLVEDWLS

SEQ ID NO: 239 - DNA, 63 bp, synthetic construct
  source 1..63: mol_type = other DNA
atgggcctcgcctttctggtgctggtggcccttgtgtggttcctggtggaagattggctgagc

SEQ ID NO: 240 - protein, 21 aa, synthetic construct
FLVIIVILSALFLGTLACFCV

SEQ ID NO: 241 - DNA, 63 bp, synthetic construct
  source 1..63: mol_type = other DNA
ttcctggtcatcatcgtgatcctgagcgccctgttcctgggcaccctggcctgtttttgcgtg

SEQ ID NO: 242 - protein, 27 aa, synthetic construct
IYIWAPLAGTCGVLLLSLVITLYCNHR

SEQ ID NO: 243 - DNA, 81 bp, synthetic construct
  source 1..81: mol_type = other DNA
atctacatctgggcccctctggctggaacatgtggtgtcctgctgctgagcctggtcatcaccctgtactgcaaccaccgg

SEQ ID NO: 244 - protein, 21 aa, synthetic construct
VAAILGLGLVLGLLGPLAILL

SEQ ID NO: 245 - DNA, 63 bp, synthetic construct
  source 1..63: mol_type = other DNA
gtggccgccattctcggactgggacttgttctgggactgctgggacctctggccattctgctg

SEQ ID NO: 246 - protein, 42 aa, synthetic construct
AAAIEVMYPPPYLDNEKSNGTIIHVKGKHLCPSPLFPGPSKP

SEQ ID NO: 247 - DNA, 126 bp, synthetic construct
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 248 - protein, 12 aa, synthetic construct
ESKYGPPCPSCP

SEQ ID NO: 249 - DNA, 36 bp, synthetic construct
  source 1..36: mol_type = other DNA
gaaagcaagtacggtccaccttgccctagctgtccg

SEQ ID NO: 250 - protein, 12 aa, synthetic construct
ESKYGPPAPSAP

SEQ ID NO: 251 - DNA, 36 bp, synthetic construct
  source 1..36: mol_type = other DNA
gaatccaagtacggccccccagcgcctagtgcccca

SEQ ID NO: 252 - protein, 12 aa, synthetic construct
ESKYGPPCPPCP

SEQ ID NO: 253 - DNA, 36 bp, synthetic construct
  source 1..36: mol_type = other DNA
gaatctaaatatggcccgccatgcccgccttgccca

SEQ ID NO: 254 - protein, 12 aa, synthetic construct
EPKSCDKTHTCP

SEQ ID NO: 255 - DNA, 36 bp, synthetic construct
  source 1..36: mol_type = other DNA
gaaccgaagtcttgtgataaaactcatacgtgcccg

SEQ ID NO: 256 - protein, 86 aa, synthetic construct
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 257 - DNA, 258 bp, synthetic construct
  source 1..258: mol_type = other DNA
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 258 - protein, 132 aa, synthetic construct
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 259 - DNA, 396 bp, synthetic construct
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 260 - protein, 34 aa, synthetic construct
ACPTGLYTHSGECCKACNLGEGVAQPCGANQTVC

SEQ ID NO: 261 - DNA, 102 bp, synthetic construct
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 262 - protein, 20 aa, synthetic construct
AVGQDTQEVIVVPHSLPFKV

SEQ ID NO: 263 - DNA, 60 bp, synthetic construct
  source 1..60: mol_type = other DNA
gctgtgggccaggacacgcaggaggtcatcgtggtgccacactccttgccctttaaggtg

SEQ ID NO: 264 - protein, 279 aa, synthetic construct
YPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGP

SEQ ID NO: 265 - protein, 39 aa, synthetic construct
KSRQTPPLASVEMEAMEALPVTWGTSSRDEDLENCSHHL

SEQ ID NO: 266 - DNA, 117 bp, synthetic construct
  source 1..117: mol_type = other DNA
aagtccagacagacacctcctctggccagcgtggaaatggaagccatggaagctctgcctgtgacctggggcaccagctccagagatgaggacctggaaaactgctcccaccacctg

SEQ ID NO: 267 - protein, 41 aa, synthetic construct
RSKRSRLLHSDYMNMTPRRPGPTRKHYQPYAPPRDFAAYRS

SEQ ID NO: 268 - DNA, 123 bp, synthetic construct
  source 1..123: mol_type = other DNA
cggagcaagagaagcagactgctgcacagcgactacatgaacatgacccctagacggcccggacctaccagaaagcactaccagccttacgctcctcctagagacttcgccgcctaccggtcc

SEQ ID NO: 269 - protein, 42 aa, synthetic construct
ALYLLRRDQRLPPDAHKPPGGGSFRTPIQEEQADAHSTLAKI

SEQ ID NO: 270 - DNA, 126 bp, synthetic construct
  source 1..126: mol_type = other DNA
gctctgtatctgctgcggagggaccaaagactgcctcctgatgctcacaagcctccaggcggaggcagcttcagaacccctatccaagaggaacaggccgacgctcacagcaccctggccaagatt

SEQ ID NO: 271 - protein, 42 aa, synthetic construct
KRGRKKLLYIFKQPFMRPVQTTQEEDGCSCRFPEEEEGGCEL

SEQ ID NO: 272 - DNA, 126 bp, synthetic construct
  source 1..126: mol_type = other DNA
aagcggggcagaaagaagctgctgtacatcttcaagcagcccttcatgcggcccgtgcagaccacacaagaggaagatggctgctcctgcagattccccgaggaagaagaaggcggctgcgagctg

SEQ ID NO: 273 - protein, 25 aa, synthetic construct
RKRTRERASRASTWEGRRRLNTQTL

SEQ ID NO: 274 - DNA, 75 bp, synthetic construct
  source 1..75: mol_type = other DNA
cggaagcggacaagagagagagccagcagagcctctacctgggagggaagaagaaggctgaacacccagacactc

SEQ ID NO: 275 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 276 - DNA, 360 bp, synthetic construct
  source 1..360: mol_type = other DNA
tggcgccgcaagcggaaagagaagcagtctgagacaagccccaaagagttcctgaccatctacgaggacgtgaaggacctgaaaacccggcggaaccacgagcaagagcagacctttcctggcggcggaagcaccatctacagcatgatccagagccagtctagcgcccctaccagccaagagcctgcctacacactgtactccctgatccagcctagcagaaagagcggcagccggaagagaaatcacagccccagcttcaacagcacgatctacgaagtgatcggcaagagccagccaaaggctcagaaccctgccaggctgagccggaaagagctggaaaacttcgacgtgtacagc

SEQ ID NO: 277 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 278 - DNA, 336 bp, synthetic construct
  source 1..336: mol_type = other DNA
agagtgaagttcagcagaagcgccgacgcacccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 279 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 280 - DNA, 336 bp, synthetic construct
  source 1..336: mol_type = other DNA
agagtgaagttcagcagatccgccgatgctcccgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaaga

SEQ ID NO: 281 - protein, 44 aa, synthetic construct
GSGQCTNYALLKLAGDVESNPGPGSGEGRGSLLTCGDVEENPGP

SEQ ID NO: 282 - DNA, 132 bp, synthetic construct
  source 1..132: mol_type = other DNA
ggtagcggccagtgtaccaactacgccctgctgaaactggccggcgacgtggaatctaatcctggacctggatctggcgagggacgcgggagtctactgacgtgtggagacgtggaggaaaaccctggacct

SEQ ID NO: 283 - protein, 41 aa, synthetic construct
QCTNYALLKLAGDVESNPGPGSGEGRGSLLTCGDVEENPGP

SEQ ID NO: 284 - DNA, 123 bp, synthetic construct
  source 1..123: mol_type = other DNA
cagtgtaccaactacgccctgctgaaactggccggcgacgtggaatctaatcctggacctggatctggcgagggacgcgggagtctactgacgtgtggagacgtggaggaaaaccctggacct

SEQ ID NO: 285 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 286 - DNA, 342 bp, synthetic construct
  source 1..342: mol_type = other DNA
aattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagc

SEQ ID NO: 287 - protein, 37 aa, synthetic construct
SGGGGSGGGGSGVTPEPIFSLIGGGSGGGGSGGGSLQ

SEQ ID NO: 288 - DNA, 111 bp, synthetic construct
  source 1..111: mol_type = other DNA
tctggcggcggaggatctggcggaggtggaagcggagttacacccgagcctatcttcagcctgatcggaggcggtagcggaggcggaggaagtggtggcggatctctgcaa

SEQ ID NO: 289 - protein, 36 aa, synthetic construct
PRAEALKGGSGGGGSGGGGSGGGGSGGGGSGGGSLQ

SEQ ID NO: 290 - protein, 41 aa, synthetic construct
SGGGGSGGGGSGITQGLAVSTISSFFGGGSGGGGSGGGSLQ

SEQ ID NO: 291 - DNA, 123 bp, synthetic construct
  source 1..123: mol_type = other DNA
agcggcggaggtggtagcggaggcggaggatctggaattacacagggactcgccgtgtctacaatctccagcttctttggtggcggtagtggcggcggtggcagtggcggtggatctcttcaa

SEQ ID NO: 292 - DNA, 109 bp, synthetic construct
  source 1..109: mol_type = other DNA
cccagagccgaggctctgaaaggcggatcaggcggcggtggtagtggaggcggaggctcaggcggcggaggttccggaggtggcggttccggcggaggatctcttcaat

SEQ ID NO: 293 - protein, 541 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGSGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 294 - DNA, 1623 bp, synthetic construct
  source 1..1623: mol_type = other DNA
atgtgtcaccagcagctggtcatcagctggttcagcctggtgttcctggcctctcctctggtggccatctgggagctgaagaaagacgtgtacgtggtggaactggactggtatcccgatgctcctggcgagatggtggtgctgacctgcgatacccctgaagaggacggcatcacctggacactggatcagtctagcgaggtgctcggcagcggcaagaccctgaccatccaagtgaaagagtttggcgacgccggccagtacacctgtcacaaaggcggagaagtgctgagccacagcctgctgctgctccacaagaaagaggatggcatttggagcaccgacatcctgaaggaccagaaagagcccaagaacaagaccttcctgagatgcgaggccaagaactacagcggccggttcacatgttggtggctgaccaccatcagcaccgacctgaccttcagcgtgaagtccagcagaggcagcagtgatcctcagggcgttacatgtggcgccgctacactgtctgccgaaagagtgcggggcgacaacaaagaatacgagtacagcgtggaatgccaagaggacagcgcctgtccagccgccgaagagtctctgcctatcgaagtgatggtggacgccgtgcacaagctgaagtacgagaactacacctccagctttttcatccgggacatcatcaagcccgatcctccaaagaacctgcagctgaagcctctgaagaacagcagacaggtggaagtgtcctgggagtaccccgacacctggtctacaccccacagctacttcagcctgaccttttgcgtgcaagtgcagggcaagtccaagcgcgagaaaaaggaccgggtgttcaccgacaagaccagcgccaccgtgatctgcagaaagaacgccagcatcagcgtcagagcccaggaccggtactacagcagctcttggagcgaatgggccagcgtgccatgttctggcggaggaagcggtggcggatcaggtggtggatctggcggcggatctagaaacctgcctgtggccactcctgatcctggcatgttcccttgtctgcaccacagccagaacctgctgagagccgtgtccaacatgctgcagaaggccagacagaccctggaattctacccctgcaccagcgaggaaatcgaccacgaggacatcaccaaggataagaccagcaccgtggaagcctgcctgcctctggaactgaccaagaacgagagctgcctgaacagccgggaaaccagcttcatcaccaacggctcttgcctggccagcagaaagacctccttcatgatggccctgtgcctgagcagcatctacgaggacctgaagatgtaccaggtggaattcaagaccatgaacgccaagctgctgatggaccccaagcggcagatcttcctggaccagaatatgctggccgtgatcgacgagctgatgcaggccctgaacttcaacagcgagacagtgccccagaagtctagcctggaagaacccgacttctacaagaccaagatcaagctgtgcatcctgctgcacgccttccggatcagagccgtgaccatcgacagagtgatgagctacctgaacgcctct

SEQ ID NO: 295 - DNA, 330 bp, synthetic construct
  source 1..330: mol_type = other DNA
gtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaa

SEQ ID NO: 296 - protein, 8 aa, synthetic construct
MPKKKRKV

SEQ ID NO: 297 - DNA, 24 bp, synthetic construct
  source 1..24: mol_type = other DNA
atgcccaagaagaagcggaaggtt

SEQ ID NO: 298 - DNA, 264 bp, synthetic construct
  source 1..264: mol_type = other DNA
aattaacgggtttcgtaacaatcgcatgaggattcgcaacgcctttgaagcagtcgacgccgaagtcccgtctcagtaaaggttgaagcagtcgacgccgaagaatcggactgccttcgtatgaagcagtcgacgccgaaggtatcagtcgcctcggaatgaagcagtcgacgccgaagattcgtaagaggctcactctcccttacacggagtggataactagttctagagggtatataatgggggccaacgcgtaccggtgtc

SEQ ID NO: 299 - DNA, 243 bp, synthetic construct
  source 1..243: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagttctagagggtatataatgggggcca

SEQ ID NO: 300 - DNA, 266 bp, synthetic construct
  source 1..266: mol_type = other DNA
acaatggctggcccatagtaaatgccgtgttagtgtgttagttgctgttcttccacgtcagaagaggcacagacaaattaccaccaggtggcgctcagagtctgcggaggcatcacaacagccctgaatttgaatcctgctctgccactgcctagttgagaccttttactacctgactagctgagacatttacgacatttactggctctaggactcattttattcatttcattactttttttttctttgagacggaatctcgctct

SEQ ID NO: 301 - protein, 763 aa, synthetic construct
MPKKKRKVDALDDFDLDMLGSDALDDFDLDMLGSDALDDFDLDMLGSDALDDFDLDMLINSRSSGSPKKKRKVGSGGGSGGSGSVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSGGGSGGSGSDLSHPPPRGHLDELTTTLESMTEDLNLDSPLTPELNEILDTFLNDECLLHAMHISTGLSIFDTSLFEDVVCCHSIYGKKKGDIDTYRYIGSSGTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHMSRPGERPFQCRICMRNFSNMSNLTRHTRTHTGEKPFQCRICMRNFSDRSVLRRHLRTHTGSQKPFQCRICMRNFSDPSNLARHTRTHTGEKPFQCRICMRNFSDRSSLRRHLRTHTGSQKPFQCRICMRNFSQSGTLHRHTRTHTGEKPFQCRICMRNFSQRPNLTRHLRTHLRGS

SEQ ID NO: 302 - protein, 763 aa, synthetic construct
MPKKKRKVMSRPGERPFQCRICMRNFSNMSNLTRHTRTHTGEKPFQCRICMRNFSDRSVLRRHLRTHTGSQKPFQCRICMRNFSDPSNLARHTRTHTGEKPFQCRICMRNFSDRSSLRRHLRTHTGSQKPFQCRICMRNFSQSGTLHRHTRTHTGEKPFQCRICMRNFSQRPNLTRHLRTHLRGSEDVVCCHSIYGKKKGDIDTYRYIGSSGTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHDALDDFDLDMLGSDALDDFDLDMLGSDALDDFDLDMLGSDALDDFDLDMLINSRSSGSPKKKRKVGSGGGSGGSGSVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSGGGSGGSGSDLSHPPPRGHLDELTTTLESMTEDLNLDSPLTPELNEILDTFLNDECLLHAMHISTGLSIFDTSLF

SEQ ID NO: 303 - protein, 762 aa, synthetic construct
MPKKKRKVSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGSEDVVCCHSIYGKKKGDIDTYRYIGSSGTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQHDALDDFDLDMLGSDALDDFDLDMLGSDALDDFDLDMLGSDALDDFDLDMLINSRSSGSPKKKRKVGSGGGSGGSGSVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSGGGSGGSGSDLSHPPPRGHLDELTTTLESMTEDLNLDSPLTPELNEILDTFLNDECLLHAMHISTGLSIFDTSLF

SEQ ID NO: 304 - DNA, 2289 bp, synthetic construct
  source 1..2289: mol_type = other DNA
atgcccaagaagaagcggaaggtttcccggcctggcgagaggcctttccagtgcagaatctgcatgcggaacttcagcagacggcacggcctggacagacacaccagaacacacacaggcgagaaacccttccagtgccggatctgtatgagaaatttcagcgaccacagcagcctgaagcggcacctgagaacccataccggcagccagaaaccatttcagtgtaggatatgcatgcgcaatttctccgtgcggcacaacctgaccagacacctgaggacacacaccggggagaagccttttcaatgtcgcatatgcatgagaaacttctctgaccactccaacctgagccgccacctcaaaacccacaccggctctcaaaagcccttccaatgtagaatatgtatgaggaactttagccagcggagcagcctcgtgcgccatctgagaactcacactggcgaaaagccgtttcaatgccgtatctgtatgcgcaactttagcgagagcggccacctgaagagacatctgcgcacacacctgagaggcagcgaggatgtcgtgtgctgccacagcatctacggaaagaagaagggcgacatcgacacctatcggtacatcggcagcagcggcacaggctgtgttgtgatcgtgggcagaatcgtgctgagcggctctggaacaagcgcccctatcacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggcacaagaacaatcgcctctccaaagggccccgtgatccagatgtacaccaacgtggaccaggacctcgttggctggcctgctcctcaaggcagcagaagcctgacaccttgcacctgtggctccagcgatctgtacctggtcaccagacacgccgacgtgatccctgtcagaagaagaggggattccagaggcagcctgctgagccctagacctatcagctacctgaagggcagctctggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcacgacgctcttgatgactttgacctggatatgctcggatcagatgccctggacgatttcgatctggacatgttggggtctgatgctctcgacgacttcgatctggatatgcttggaagtgacgcgctggatgatttcgaccttgacatgctcatcaattctcgatccagtggaagcccgaaaaagaaacgcaaggtgggaagtgggggcggctccggtgggagcggtagtgtattgcctcaagctcccgcgcccgctcctgctccggcaatggtttcagctctggcacaagctccagctccagtgcctgtgctcgcccctggccctccgcaggccgtagcacctcccgcccccaaaccgacgcaagccggtgaggggactctctctgaagccttgctgcagcttcagttcgatgatgaagatctgggcgcgctcttggggaacagcacggatccggcagtatttacggacctcgcatcagttgacaatagtgaatttcaacaacttcttaaccagggaataccggttgcgccccatacgacggaacctatgctgatggagtaccctgaagctataaccagactcgtaactggcgcccaacgcccgcccgacccggctcctgcgccgctgggtgcgccgggtcttccgaatggtcttctctcaggggacgaagatttcagttccattgcggatatggacttttccgcgctcctgagtgggggtggctctggaggctctggttccgacctcagccatcctccaccgagaggacacctcgacgagctgacaaccaccctcgaaagtatgacggaagatctgaacttggattccccccttaccccagaactgaatgaaatcctcgatacgttcttgaacgatgagtgccttttgcacgccatgcatatatcaacaggtttgtctatcttcgacacgtccctcttttga

SEQ ID NO: 305 - DNA, 2289 bp, synthetic construct
  source 1..2289: mol_type = other DNA
atgcccaagaaaaagcggaaggtgatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctgaggatgtcgtgtgctgccacagcatctacggcaagaagaagggcgacatcgacacctaccggtacatcggcagctctggcacaggctgtgtggtcatcgtgggcagaatcgtgctgtctggcagcggaacaagcgcccctatcacagcctatgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggccccgtgatccagatgtacaccaacgtggaccaggacctcgttggctggcctgctcctcaaggcagcagaagcctgacaccttgcacctgtggctccagcgatctgtacctggtcaccagacacgccgacgtgatccctgtcagaagaagaggggattccagaggcagcctgctgagccctagacctatcagctacctgaagggctctagcggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcacgacgccctggacgacttcgatctggatatgctgggcagcgacgctctggatgattttgacctggacatgctcggctctgatgcactcgacgatttcgacctcgatatgttgggatctgatgcccttgatgactttgatctcgacatgttgatcaatagccggtccagcggcagccccaagaagaagagaaaagtcggctctggcggcggatctggcggttctggatctgttttgccccaagctcctgctcctgcaccagctccagctatggtttctgctctggctcaggctccagctcctgtgcctgttcttgctcctggacctcctcaggctgttgctccaccagcacctaaacctacacaggccggcgagggaacactgtctgaagctctgctgcagctccagttcgacgacgaagatctgggagccctgctgggcaatagcacagatcctgccgtgttcaccgatctggccagcgtggacaatagcgagttccagcagctcctgaaccagggcattcctgtggctcctcacaccaccgagcctatgctgatggaataccccgaggccatcaccagactggtcaccggtgctcaaagaccacctgatccggctccagcacctcttggagcacctggactgcctaatggactgctgtctggcgacgaggacttcagctctatcgccgacatggatttcagcgccctgctcagtggcggtggaagcggaggaagtggcagcgatctttctcaccctccacctagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttt

SEQ ID NO: 306 - DNA, 2289 bp, synthetic construct
  source 1..2289: mol_type = other DNA
atgcccaagaaaaagcggaaggtggacgccctggacgacttcgatctggatatgctgggcagcgacgctctggatgattttgacctggacatgctcggctctgatgcactcgacgatttcgacctcgatatgttgggatctgatgcccttgatgactttgatctcgacatgttgatcaatagccggtccagcggcagccccaagaagaagagaaaagtcggctctggcggcggatctggcggttctggatctgttttgccccaagctcctgctcctgcaccagctccagctatggtttctgctctggctcaggctccagctcctgtgcctgttcttgctcctggacctcctcaggctgttgctccaccagcacctaaacctacacaggccggcgagggaacactgtctgaagctctgctgcagctccagttcgacgacgaagatctgggagccctgctgggcaatagcacagatcctgccgtgttcaccgatctggccagcgtggacaatagcgagttccagcagctcctgaaccagggcattcctgtggctcctcacaccaccgagcctatgctgatggaataccccgaggccatcaccagactggtcaccggtgctcaaagaccacctgatccggctccagcacctcttggagcacctggactgcctaatggactgctgtctggcgacgaggacttcagctctatcgccgacatggatttcagcgccctgctcagtggcggtggaagcggaggaagtggcagcgatctttctcaccctccacctagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgtttgaggatgtcgtgtgctgccacagcatctacggcaagaagaagggcgacatcgacacctaccggtacatcggcagctctggcacaggctgtgtggtcatcgtgggcagaatcgtgctgtctggcagcggaacaagcgcccctatcacagcctatgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggccccgtgatccagatgtacaccaacgtggaccaggacctcgttggctggcctgctcctcaaggcagcagaagcctgacaccttgcacctgtggctccagcgatctgtacctggtcaccagacacgccgacgtgatccctgtcagaagaagaggggattccagaggcagcctgctgagccctagacctatcagctacctgaagggctctagcggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcacatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatct

SEQ ID NO: 307 - DNA, 1332 bp, synthetic construct
  source 1..1332: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatatcgtggtgggcgtggtgtgcaccctgctggtggccctgctgatggccgccctgtacctggtgagaatcagacagaagaaggcccagggcagcaccagcagcaccagactgcacgagcccgagaagaacgccagagagatcacccaggacaccaacgacatcacctacgccgacctgaacctgcccaagggcaagaagcccgccccccaggccgccgagcccaacaaccacaccgagtacgccagcatccagaccagcccccagcccgccagcgaggacaccctgacctacgccgacctggacatggtgcacctgaacagaacccccaagcagcccgcccccaagcccgagcccagcttcagcgagtacgccagcgtgcaggtgcccagaaag

SEQ ID NO: 308 - DNA, 8250 bp, synthetic construct
  source 1..8250: mol_type = other DNA
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagaagccagtttgcatctgtcaggatcaatttcccattatgccagtcatattaattactagtcaattagttgatttttatttttgacatatacatgtgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatggatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatccgccgccaccatgctgctgctggtcacatctctgctgctgtgcgagctgccccatcctgcctttctgctgatccctcacatggaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccaggttcaccatctccagagatgacagcaagaacagcctgtacctgcagatgaactccctgaaaaccgaggacaccgccgtgtactattgcgtggccggcaatagctttgcctactggggacagggcaccctggttacagtttctgctggcggcggaggaagcggaggcggaggatccggtggtggtggatctgacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaaatctggcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgcaggcggagccgtgcatacaagaggactggatttcgcctgcgacatctacatctgggcccctctggctggaacatgtggtgtcctgctgctgagcctggtcatcaccctgtactgcaaccaccggcggagcaagagaagcagactgctgcacagcgactacatgaacatgacccctagacggcccggacctaccagaaagcactaccagccttacgctcctcctagagacttcgccgcctaccggtccagagtgaagttcagcagatccgccgatgctcccgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggtagcggccagtgtaccaactacgccctgctgaaactggccggcgacgtggaatctaatcctggacctggatctggcgagggacgcgggagtctactgacgtgtggagacgtggaggaaaaccctggacctatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgtttaaagatctagatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 309 - DNA, 9461 bp, synthetic construct
  source 1..9461: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgaggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggggccgccaccatgctgctgctggtcacatctctgctgctgtgcgagctgccccatcctgcctttctgctgatccctcacatggacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtactactgccagcagtactacaactaccctctgaccttcggccagggcaccaagctggaaatcaaaggcggcggaggatctggcggaggtggaagtggcggaggcggatctgaagtgcagctggttgaatcaggtggcggcctggttcaacctggcggatctctgagactgagctgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccagattcaccatcagccgggacgacagcaagaacagcctgtacctgcagatgaactccctgaaaaccgaggacaccgccgtgtattattgcgtggccggcaacagctttgcctactggggacagggaaccctggtcaccgtgtctgccacaacaacccctgctcctagacctcctacaccagctcctacaatcgccctgcagcctctgtctctgaggccagaagcttgtagaccagctgctggcggagccgtgcatacaagaggactggacttcgcctgtgatgtggccgccattctcggactgggacttgttctgggactgctgggacctctggccattctgctggctctgtatctgctgcggagggaccaaagactgcctcctgatgctcacaagcctccaggcggaggcagcttcagaacccctatccaagaggaacaggccgacgctcacagcaccctggccaagattagagtgaagttcagcagaagcgccgacgcacccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggtagcggccagtgtaccaactacgccctgctgaaactggccggcgacgtggaatctaatcctggacctggatctggcgagggacgcgggagtctactgacgtgtggagacgtggaggaaaaccctggacctatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgtttaaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 310 - DNA, 9545 bp, synthetic construct
  source 1..9545: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgaggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggggccgccaccatgctgctgctggtcacatctctgctgctgtgcgagctgccccatcctgcctttctgctgatccctcacatggaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccaggttcaccatctccagagatgacagcaagaacagcctgtacctgcagatgaactccctgaaaaccgaggacaccgccgtgtactattgcgtggccggcaatagctttgcctactggggacagggcaccctggttacagtttctgctggcggcggaggaagcggaggcggaggatccggtggtggtggatctgacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaaatctggcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgcaggcggagccgtgcatacaagaggactggatttcgcctgcgacatctacatctgggcccctctggctggaacatgtggtgtcctgctgctgagcctggtcatcaccctgtactgcaaccaccggcggagcaagagaagcagactgctgcacagcgactacatgaacatgacccctagacggcccggacctaccagaaagcactaccagccttacgctcctcctagagacttcgccgcctaccggtccagagtgaagttcagcagatccgccgatgctcccgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggtagcggccagtgtaccaactacgccctgctgaaactggccggcgacgtggaatctaatcctggacctggatctggcgagggacgcgggagtctactgacgtgtggagacgtggaggaaaaccctggacctatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgtttaaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 311 - DNA, 9536 bp, synthetic construct
  source 1..9536: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgaggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggggccgccaccatgctgctgctggtcacatctctgctgctgtgcgagctgccccatcctgcctttctgctgatccctcacatggaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccaggttcaccatctccagagatgacagcaagaacagcctgtacctgcagatgaactccctgaaaaccgaggacaccgccgtgtactattgcgtggccggcaatagctttgcctactggggacagggcaccctggttacagtttctgctggcggcggaggaagcggaggcggaggatccggtggtggtggatctgacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaaatctggcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgcaggcggagccgtgcatacaagaggactggatttcgcctgcgacatctacatctgggcccctctggctggaacatgtggtgtcctgctgctgagcctggtcatcaccctgtactgcaaccaccggcggagcaagagaagcagactgctgcacagcgactacatgaacatgacccctagacggcccggacctaccagaaagcactaccagccttacgctcctcctagagacttcgccgcctaccggtccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgtaccaactacgccctgctgaaactggccggcgacgtggaatctaatcctggacctggatctggcgagggacgcgggagtctactgacgtgtggagacgtggaggaaaaccctggacctatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgtttaaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 312 - DNA, 8166 bp, synthetic construct
  source 1..8166: mol_type = other DNA
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagaagccagtttgcatctgtcaggatcaatttcccattatgccagtcatattaattactagtcaattagttgatttttatttttgacatatacatgtgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatggatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatccgccgccaccatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgttggtagcggccagtgtaccaactacgccctgctgaaactggccggcgacgtggaatctaatcctggacctggatctggcgagggacgcgggagtctactgacgtgtggagacgtggaggaaaaccctggacctatgctgctgctggtcacatctctgctgctgtgcgagctgccccatcctgcctttctgctgatccctcacatggacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtactactgccagcagtactacaactaccctctgaccttcggccagggcaccaagctggaaatcaaaggcggcggaggatctggcggaggtggaagtggcggaggcggatctgaagtgcagctggttgaatcaggtggcggcctggttcaacctggcggatctctgagactgagctgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccagattcaccatcagccgggacgacagcaagaacagcctgtacctgcagatgaactccctgaaaaccgaggacaccgccgtgtattattgcgtggccggcaacagctttgcctactggggacagggaaccctggtcaccgtgtctgccacaacaacccctgctcctagacctcctacaccagctcctacaatcgccctgcagcctctgtctctgaggccagaagcttgtagaccagctgctggcggagccgtgcatacaagaggactggacttcgcctgtgatgtggccgccattctcggactgggacttgttctgggactgctgggacctctggccattctgctggctctgtatctgctgcggagggaccaaagactgcctcctgatgctcacaagcctccaggcggaggcagcttcagaacccctatccaagaggaacaggccgacgctcacagcaccctggccaagattagagtgaagttcagcagaagcgccgacgcacccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataaagatctagatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 313 - DNA, 8250 bp, synthetic construct
  source 1..8250: mol_type = other DNA
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagaagccagtttgcatctgtcaggatcaatttcccattatgccagtcatattaattactagtcaattagttgatttttatttttgacatatacatgtgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatggatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatccgccgccaccatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgttggtagcggccagtgtaccaactacgccctgctgaaactggccggcgacgtggaatctaatcctggacctggatctggcgagggacgcgggagtctactgacgtgtggagacgtggaggaaaaccctggacctatgctgctgctggtcacatctctgctgctgtgcgagctgccccatcctgcctttctgctgatccctcacatggaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccaggttcaccatctccagagatgacagcaagaacagcctgtacctgcagatgaactccctgaaaaccgaggacaccgccgtgtactattgcgtggccggcaatagctttgcctactggggacagggcaccctggttacagtttctgctggcggcggaggaagcggaggcggaggatccggtggtggtggatctgacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaaatctggcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgcaggcggagccgtgcatacaagaggactggatttcgcctgcgacatctacatctgggcccctctggctggaacatgtggtgtcctgctgctgagcctggtcatcaccctgtactgcaaccaccggcggagcaagagaagcagactgctgcacagcgactacatgaacatgacccctagacggcccggacctaccagaaagcactaccagccttacgctcctcctagagacttcgccgcctaccggtccagagtgaagttcagcagatccgccgatgctcccgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataaagatctagatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 314 - DNA, 8157 bp, synthetic construct
  source 1..8157: mol_type = other DNA
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagaagccagtttgcatctgtcaggatcaatttcccattatgccagtcatattaattactagtcaattagttgatttttatttttgacatatacatgtgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatggatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccggatccgccgccaccatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgttcagtgtaccaactacgccctgctgaaactggccggcgacgtggaatctaatcctggacctggatctggcgagggacgcgggagtctactgacgtgtggagacgtggaggaaaaccctggacctatgctgctgctggtcacatctctgctgctgtgcgagctgccccatcctgcctttctgctgatccctcacatggacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtactactgccagcagtactacaactaccctctgaccttcggccagggcaccaagctggaaatcaaaggcggcggaggatctggcggaggtggaagtggcggaggcggatctgaagtgcagctggttgaatcaggtggcggcctggttcaacctggcggatctctgagactgagctgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccagattcaccatcagccgggacgacagcaagaacagcctgtacctgcagatgaactccctgaaaaccgaggacaccgccgtgtattattgcgtggccggcaacagctttgcctactggggacagggaaccctggtcaccgtgtctgccacaacaacccctgctcctagacctcctacaccagctcctacaatcgccctgcagcctctgtctctgaggccagaagcttgtagaccagctgctggcggagccgtgcatacaagaggactggacttcgcctgtgatgtggccgccattctcggactgggacttgttctgggactgctgggacctctggccattctgctggctctgtatctgctgcggagggaccaaagactgcctcctgatgctcacaagcctccaggcggaggcagcttcagaacccctatccaagaggaacaggccgacgctcacagcaccctggccaagattagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaagatctagatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 315 - DNA, 9459 bp, synthetic construct
  source 1..9459: mol_type = other DNA
aagcttggaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgaggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggggccgccaccatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagccccagagccgaggctctgaaaggcggatcaggcggcggtggtagtggaggcggaggctcaggcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgggtagcggccagtgtaccaactacgccctgctgaaactggccggcgacgtggaatctaatcctggacctggatctggcgagggacgcgggagtctactgacgtgtggagacgtggaggaaaaccctggacctatgctgctgctggtcacatctctgctgctgtgcgagctgccccatcctgcctttctgctgatccctcacatggacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtactactgccagcagtactacaactaccctctgaccttcggccagggcaccaagctggaaatcaaaggcggcggaggatctggcggaggtggaagtggcggaggcggatctgaagtgcagctggttgaatcaggtggcggcctggttcaacctggcggatctctgagactgagctgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccagattcaccatcagccgggacgacagcaagaacagcctgtacctgcagatgaactccctgaaaaccgaggacaccgccgtgtattattgcgtggccggcaacagctttgcctactggggacagggaaccctggtcaccgtgtctgccacaacaacccctgctcctagacctcctacaccagctcctacaatcgccctgcagcctctgtctctgaggccagaagcttgtagaccagctgctggcggagccgtgcatacaagaggactggacttcgcctgtgatgtggccgccattctcggactgggacttgttctgggactgctgggacctctggccattctgctggctctgtatctgctgcggagggaccaaagactgcctcctgatgctcacaagcctccaggcggaggcagcttcagaacccctatccaagaggaacaggccgacgctcacagcaccctggccaagattagagtgaagttcagcagaagcgccgacgcacccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 316 - DNA, 9545 bp, synthetic construct
  source 1..9545: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgaggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggggccgccaccatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgttggtagcggccagtgtaccaactacgccctgctgaaactggccggcgacgtggaatctaatcctggacctggatctggcgagggacgcgggagtctactgacgtgtggagacgtggaggaaaaccctggacctatgctgctgctggtcacatctctgctgctgtgcgagctgccccatcctgcctttctgctgatccctcacatggaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccaggttcaccatctccagagatgacagcaagaacagcctgtacctgcagatgaactccctgaaaaccgaggacaccgccgtgtactattgcgtggccggcaatagctttgcctactggggacagggcaccctggttacagtttctgctggcggcggaggaagcggaggcggaggatccggtggtggtggatctgacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaaatctggcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgcaggcggagccgtgcatacaagaggactggatttcgcctgcgacatctacatctgggcccctctggctggaacatgtggtgtcctgctgctgagcctggtcatcaccctgtactgcaaccaccggcggagcaagagaagcagactgctgcacagcgactacatgaacatgacccctagacggcccggacctaccagaaagcactaccagccttacgctcctcctagagacttcgccgcctaccggtccagagtgaagttcagcagatccgccgatgctcccgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 317 - DNA, 12109 bp, synthetic construct
  source 1..12109: mol_type = other DNA
aagcttggaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttacacaggccgcacagattctctccgcagccgttcgtttcttctccgctctctgcaccgaggggcgaagcagtaggtcaggcaacagatcacgaagatgccgttcacggagatcagtgtgatggcccagcttggcagcagttgaagagatccaccgccactgccaccgccgccactaccgccaccaaagaagctggagattgtagacacggcgagtccctgtgtaattccagatcctccgcctccgctaccacctccgccgctagaggcgttcaggtagctcatcactctgtcgatggtcacggctctgatccggaaggcgtgcagcaggatgcacagcttgatcttggtcttgtagaagtcgggttcttccaggctagacttctggggcactgtctcgctgttgaagttcagggcctgcatcagctcgtcgatcacggccagcatattctggtccaggaagatctgccgcttggggtccatcagcagcttggcgttcatggtcttgaattccacctggtacatcttcaggtcctcgtagatgctgctcaggcacagggccatcatgaaggaggtctttctgctggccaggcaagagccgttggtgatgaagctggtttcccggctgttcaggcagctctcgttcttggtcagttccagaggcaggcaggcttccacggtgctggtcttatccttggtgatgtcctcgtggtcgatttcctcgctggtgcaggggtagaattccagggtctgtctggccttctgcagcatgttggacacggctctcagcaggttctggctgtggtgcagacaagggaacatgccaggatcaggagtggccacaggcaggtttctagatccgccgccagatccaccacctgatccgccaccgcttcctccgccagaacatggcacgctggcccattcgctccaagagctgctgtagtaccggtcctgggctctgacgctgatgctggcgttctttctgcagatcacggtggcgctggtcttgtcggtgaacacccggtcctttttctcgcgcttggacttgccctgcacttgcacgcaaaaggtcaggctgaagtagctgtggggtgtagaccaggtgtcggggtactcccaggacacttccacctgtctgctgttcttcagaggcttcagctgcaggttctttggaggatcgggcttgatgatgtcccggatgaaaaagctggaggtgtagttctcgtacttcagcttgtgcacggcgtccaccatcacttcgataggcagagactcttcggcggctggacaggcgctgtcctcttggcattccacgctgtactcgtattctttgttgtcgccccgcactctttcggcagacagtgtagcggcgccacatgtaacgccctgaggatcactgctgcctctgctggacttcacgctgaaggtcaggtcggtgctgatggtggtcagccaccaacatgtgaaccggccgctgtagttcttggcctcgcatctcaggaaggtcttgttcttgggctctttctggtccttcaggatgtcggtgctccaaatgccatcctctttcttgtggagcagcagcaggctgtggctcagcacttctccgcctttgtgacaggtgtactggccggcgtcgccaaactctttcacttggatggtcagggtcttgccgctgccgagcacctcgctagactgatccagtgtccaggtgatgccgtcctcttcaggggtatcgcaggtcagcaccaccatctcgccaggagcatcgggataccagtccagttccaccacgtacacgtctttcttcagctcccagatggccaccagaggagaggccaggaacaccaggctgaaccagctgatgaccagctgctggtgacacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgcccaagaaaaagcggaaggtggacgccctggacgacttcgatctggatatgctgggcagcgacgctctggatgattttgacctggacatgctcggctctgatgcactcgacgatttcgacctcgatatgttgggatctgatgcccttgatgactttgatctcgacatgttgatcaatagccggtccagcggcagccccaagaagaagagaaaagtcggctctggcggcggatctggcggttctggatctgttttgccccaagctcctgctcctgcaccagctccagctatggtttctgctctggctcaggctccagctcctgtgcctgttcttgctcctggacctcctcaggctgttgctccaccagcacctaaacctacacaggccggcgagggaacactgtctgaagctctgctgcagctccagttcgacgacgaagatctgggagccctgctgggcaatagcacagatcctgccgtgttcaccgatctggccagcgtggacaatagcgagttccagcagctcctgaaccagggcattcctgtggctcctcacaccaccgagcctatgctgatggaataccccgaggccatcaccagactggtcaccggtgctcaaagaccacctgatccggctccagcacctcttggagcacctggactgcctaatggactgctgtctggcgacgaggacttcagctctatcgccgacatggatttcagcgccctgctcagtggcggtggaagcggaggaagtggcagcgatctttctcaccctccacctagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgtttgaggatgtcgtgtgctgccacagcatctacggcaagaagaagggcgacatcgacacctaccggtacatcggcagctctggcacaggctgtgtggtcatcgtgggcagaatcgtgctgtctggcagcggaacaagcgcccctatcacagcctatgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggccccgtgatccagatgtacaccaacgtggaccaggacctcgttggctggcctgctcctcaaggcagcagaagcctgacaccttgcacctgtggctccagcgatctgtacctggtcaccagacacgccgacgtgatccctgtcagaagaagaggggattccagaggcagcctgctgagccctagacctatcagctacctgaagggctctagcggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcacatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatcttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 318 - DNA, 12109 bp, synthetic construct
  source 1..12109: mol_type = other DNA
aagcttggaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttacttacacaggccgcacagattctctccgcagccgttcgtttcttctccgctctctgcaccgaggggcgaagcagtaggtcaggcaacagatcacgaagatgccgttcacggagatcagtgtgatggcccagcttggcagcagttgaagagatccaccgccactgccaccgccgccactaccgccaccaaagaagctggagattgtagacacggcgagtccctgtgtaattccagatcctccgcctccgctaccacctccgccgctagaggcgttcaggtagctcatcactctgtcgatggtcacggctctgatccggaaggcgtgcagcaggatgcacagcttgatcttggtcttgtagaagtcgggttcttccaggctagacttctggggcactgtctcgctgttgaagttcagggcctgcatcagctcgtcgatcacggccagcatattctggtccaggaagatctgccgcttggggtccatcagcagcttggcgttcatggtcttgaattccacctggtacatcttcaggtcctcgtagatgctgctcaggcacagggccatcatgaaggaggtctttctgctggccaggcaagagccgttggtgatgaagctggtttcccggctgttcaggcagctctcgttcttggtcagttccagaggcaggcaggcttccacggtgctggtcttatccttggtgatgtcctcgtggtcgatttcctcgctggtgcaggggtagaattccagggtctgtctggccttctgcagcatgttggacacggctctcagcaggttctggctgtggtgcagacaagggaacatgccaggatcaggagtggccacaggcaggtttctagatccgccgccagatccaccacctgatccgccaccgcttcctccgccagaacatggcacgctggcccattcgctccaagagctgctgtagtaccggtcctgggctctgacgctgatgctggcgttctttctgcagatcacggtggcgctggtcttgtcggtgaacacccggtcctttttctcgcgcttggacttgccctgcacttgcacgcaaaaggtcaggctgaagtagctgtggggtgtagaccaggtgtcggggtactcccaggacacttccacctgtctgctgttcttcagaggcttcagctgcaggttctttggaggatcgggcttgatgatgtcccggatgaaaaagctggaggtgtagttctcgtacttcagcttgtgcacggcgtccaccatcacttcgataggcagagactcttcggcggctggacaggcgctgtcctcttggcattccacgctgtactcgtattctttgttgtcgccccgcactctttcggcagacagtgtagcggcgccacatgtaacgccctgaggatcactgctgcctctgctggacttcacgctgaaggtcaggtcggtgctgatggtggtcagccaccaacatgtgaaccggccgctgtagttcttggcctcgcatctcaggaaggtcttgttcttgggctctttctggtccttcaggatgtcggtgctccaaatgccatcctctttcttgtggagcagcagcaggctgtggctcagcacttctccgcctttgtgacaggtgtactggccggcgtcgccaaactctttcacttggatggtcagggtcttgccgctgccgagcacctcgctagactgatccagtgtccaggtgatgccgtcctcttcaggggtatcgcaggtcagcaccaccatctcgccaggagcatcgggataccagtccagttccaccacgtacacgtctttcttcagctcccagatggccaccagaggagaggccaggaacaccaggctgaaccagctgatgaccagctgctggtgacacatcatggtggcgacaccggtacgcgttggcccccattatataccctctagaactagttatccactccgtgtaagggagagtgagcctcttacgaatcttcggcgtcgactgcttcattccgaggcgactgataccttcggcgtcgactgcttcatacgaaggcagtccgattcttcggcgtcgactgcttcaacctttactgagacgggacttcggcgtcgactgcttcaaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgcccaagaaaaagcggaaggtgatgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatctgaggatgtcgtgtgctgccacagcatctacggcaagaagaagggcgacatcgacacctaccggtacatcggcagctctggcacaggctgtgtggtcatcgtgggcagaatcgtgctgtctggcagcggaacaagcgcccctatcacagcctatgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggaaccagaacaatcgcctctcctaagggccccgtgatccagatgtacaccaacgtggaccaggacctcgttggctggcctgctcctcaaggcagcagaagcctgacaccttgcacctgtggctccagcgatctgtacctggtcaccagacacgccgacgtgatccctgtcagaagaagaggggattccagaggcagcctgctgagccctagacctatcagctacctgaagggctctagcggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcacgacgccctggacgacttcgatctggatatgctgggcagcgacgctctggatgattttgacctggacatgctcggctctgatgcactcgacgatttcgacctcgatatgttgggatctgatgcccttgatgactttgatctcgacatgttgatcaatagccggtccagcggcagccccaagaagaagagaaaagtcggctctggcggcggatctggcggttctggatctgttttgccccaagctcctgctcctgcaccagctccagctatggtttctgctctggctcaggctccagctcctgtgcctgttcttgctcctggacctcctcaggctgttgctccaccagcacctaaacctacacaggccggcgagggaacactgtctgaagctctgctgcagctccagttcgacgacgaagatctgggagccctgctgggcaatagcacagatcctgccgtgttcaccgatctggccagcgtggacaatagcgagttccagcagctcctgaaccagggcattcctgtggctcctcacaccaccgagcctatgctgatggaataccccgaggccatcaccagactggtcaccggtgctcaaagaccacctgatccggctccagcacctcttggagcacctggactgcctaatggactgctgtctggcgacgaggacttcagctctatcgccgacatggatttcagcgccctgctcagtggcggtggaagcggaggaagtggcagcgatctttctcaccctccacctagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 319 - DNA, 11121 bp, synthetic construct
  source 1..11121: mol_type = other DNA
acgcgtgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccactgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaattttcgggggatcagcggaattctagagtcgcggccgctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagctgtttaaatattaaacagggaaccgatgtgtttaaactagagtcgcggcctcagtcagtcacgcatgcctgcagtttaactggcgttcaggtaggacatcacgcggtcaatggtcacggctcgaatgcggaaagcgtgcaacaggatgcacagcttgatcttggtcttgtagaagtccggttcttccaggctggacttttgaggcacagtctcggagttgaaattcagggcctgcatcagttcatcaatcacggcgagcatattctggtccaggaagatctgccgcttcgggtccatgagcagcttggcgttcatggtcttgaactcgacctgatacatcttcagatcttcgtagatcgaggaaagacagagcgccatcatgaatgaggtctttctcgacgccaggcagctgccgttagtgataaagcttgtctcgcgggagttcagacacgattcgttcttggtcagttccagcggcaggcaggcttccacggtcgaggtcttgtccttggtgatgtcctcgtgatcaatttcttccgaggtgcaggggtagaactcaagggtctggcgggccttctgcaacatgttcgacacagccctcaggaggttttgggagtggtgtaggcacgggaacattccagggtcgggggttgccacagggaggttccgggaacctcctccggagcctcctcctgaacctccgcctgatccgccaccggaacaaggcacgctggcccattcgctccaggaggacgagtagtatctatcctgcgcccggacgctgattgacgcgttcttccgacaaatcacagtggcggaggttttgtcggtgaacacccggtctttcttctcccgtttggactttccctgcacttgcacacagaaagtgagcgagaagtatgagtgcggggtgctccaagtgtctggatattcccaagacacttccacttggcgggagttcttgagtggcttcagctgcaagttcttgggggggtcaggcttgatgatgtcgcggataaagaaggaggaagtgtagttctcgtatttcagcttatgcacggcatcgaccatgacctcgataggcagggactcttccgcggcagggcaggcgctgtcctcctggcattccacggagtactcatattccttgttgtctcccctgactctctcggcggacagagtggcggctccacaggtcacgccctgaggatcgcttgatccccgtgacgacttcacggagaaagtcaggtcggtggagattgtcgtcagccaccaacaggtgaaccgaccgctgtagttcttggcttcgcagcggaggaaggtcttgttcttcggttctttttggtccttgaggatgtcagtggaccagattccatcctctttcttgtgcagcagcagcagggagtgggacagcacttcgccacccttgtggcaagtgtactggcccgcgtcgccgaactccttgacttgaatggtcagggtctttccgcttccgagcacctcggagctctgatccagggtccaggttatgccgtcctcttctggcgtatcgcaagtcagcacgaccatttctccaggggcgtccgggtaccaatccagctcgaccacgtagacgtccttcttcagttcccaaatggcgaccagaggggaagcgaggaacacaagggagaaccaggagatgacgagttgctgatggcacatcatggtggcgacaccggtacgcgttggcccccattatataccctctagaactagttatccactccgtgtaagggagagtgagcctcttacgaatgcgcgacatcggctacgccgttccgaggcgactgatacgcgcgacatcggctacgccgtacgaaggcagtccgattgcgcgacatcggctacgccgacctttactgagacgggagcgcgacatcggctacgccgaaggcgttgcgaatcctcatgcgattgttacgaaacccgttaattaaagagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgtccatctagatggccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggatccgccaccatgcccaagaagaagcggaaggtttcccggcctggcgagaggcctttccagtgcagaatctgcatgcggaacttcagcagacggcacggcctggacagacacaccagaacacacacaggcgagaaacccttccagtgccggatctgtatgagaaatttcagcgaccacagcagcctgaagcggcacctgagaacccataccggcagccagaaaccatttcagtgtaggatatgcatgcgcaatttctccgtgcggcacaacctgaccagacacctgaggacacacaccggggagaagccttttcaatgtcgcatatgcatgagaaacttctctgaccactccaacctgagccgccacctcaaaacccacaccggctctcaaaagcccttccaatgtagaatatgtatgaggaactttagccagcggagcagcctcgtgcgccatctgagaactcacactggcgaaaagccgtttcaatgccgtatctgtatgcgcaactttagcgagagcggccacctgaagagacatctgcgcacacacctgagaggcagcgaggatgtcgtgtgctgccacagcatctacggaaagaagaagggcgacatcgacacctatcggtacatcggcagcagcggcacaggctgtgttgtgatcgtgggcagaatcgtgctgagcggctctggaacaagcgcccctatcacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggcacaagaacaatcgcctctccaaagggccccgtgatccagatgtacaccaacgtggaccaggacctcgttggctggcctgctcctcaaggcagcagaagcctgacaccttgcacctgtggctccagcgatctgtacctggtcaccagacacgccgacgtgatccctgtcagaagaagaggggattccagaggcagcctgctgagccctagacctatcagctacctgaagggcagctctggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcacgacgctcttgatgactttgacctggatatgctcggatcagatgccctggacgatttcgatctggacatgttggggtctgatgctctcgacgacttcgatctggatatgcttggaagtgacgcgctggatgatttcgaccttgacatgctcatcaattctcgatccagtggaagcccgaaaaagaaacgcaaggtgggaagtgggggcggctccggtgggagcggtagtgtattgcctcaagctcccgcgcccgctcctgctccggcaatggtttcagctctggcacaagctccagctccagtgcctgtgctcgcccctggccctccgcaggccgtagcacctcccgcccccaaaccgacgcaagccggtgaggggactctctctgaagccttgctgcagcttcagttcgatgatgaagatctgggcgcgctcttggggaacagcacggatccggcagtatttacggacctcgcatcagttgacaatagtgaatttcaacaacttcttaaccagggaataccggttgcgccccatacgacggaacctatgctgatggagtaccctgaagctataaccagactcgtaactggcgcccaacgcccgcccgacccggctcctgcgccgctgggtgcgccgggtcttccgaatggtcttctctcaggggacgaagatttcagttccattgcggatatggacttttccgcgctcctgagtgggggtggctctggaggctctggttccgacctcagccatcctccaccgagaggacacctcgacgagctgacaaccaccctcgaaagtatgacggaagatctgaacttggattccccccttaccccagaactgaatgaaatcctcgatacgttcttgaacgatgagtgccttttgcacgccatgcatatatcaacaggtttgtctatcttcgacacgtccctcttttgagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtctacgccttcgccctcagacgagtcggatctccctttgggccgcctccccgcctggtacctttaagaccaatgacttacaaggcagctgtagatcttagccactttttaaaagaaaaggggggactggaagggctaattcactcccaacgaaaataagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctagacttttgcagagacggcccaaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctg

SEQ ID NO: 320 - protein, 177 aa, synthetic construct
MSRPGERPFQCRICMRNFSNMSNLTRHTRTHTGEKPFQCRICMRNFSDRSVLRRHLRTHTGSQKPFQCRICMRNFSDPSNLARHTRTHTGEKPFQCRICMRNFSDRSSLRRHLRTHTGSQKPFQCRICMRNFSQSGTLHRHTRTHTGEKPFQCRICMRNFSQRPNLTRHLRTHLRGS

SEQ ID NO: 321 - protein, 265 aa, synthetic construct
EDVVCCHSIYGKKKGDIDTYRYIGSSGTGCVVIVGRIVLSGSGTSAPITAYAQQTRGLLGCIITSLTGRDKNQVEGEVQIVSTATQTFLATCINGVCWAVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLETTMRSPVFTDNSSPPAVTLTHPITKIDREVLYQEFDEMEECSQH

SEQ ID NO: 322 - DNA, 939 bp, synthetic construct
  source 1..939: mol_type = other DNA
gacgccctggacgacttcgatctggatatgctgggcagcgacgctctggatgattttgacctggacatgctcggctctgatgcactcgacgatttcgacctcgatatgttgggatctgatgcccttgatgactttgatctcgacatgttgatcaatagccggtccagcggcagccccaagaagaagagaaaagtcggctctggcggcggatctggcggttctggatctgttttgccccaagctcctgctcctgcaccagctccagctatggtttctgctctggctcaggctccagctcctgtgcctgttcttgctcctggacctcctcaggctgttgctccaccagcacctaaacctacacaggccggcgagggaacactgtctgaagctctgctgcagctccagttcgacgacgaagatctgggagccctgctgggcaatagcacagatcctgccgtgttcaccgatctggccagcgtggacaatagcgagttccagcagctcctgaaccagggcattcctgtggctcctcacaccaccgagcctatgctgatggaataccccgaggccatcaccagactggtcaccggtgctcaaagaccacctgatccggctccagcacctcttggagcacctggactgcctaatggactgctgtctggcgacgaggacttcagctctatcgccgacatggatttcagcgccctgctcagtggcggtggaagcggaggaagtggcagcgatctttctcaccctccacctagaggccacctggacgagctgacaaccacactggaatccatgaccgaggacctgaacctggacagccctctgacacccgagctgaacgagatcctggacaccttcctgaacgacgagtgtctgctgcacgccatgcacatctctaccggcctgagcatcttcgacaccagcctgttt

SEQ ID NO: 323 - DNA, 531 bp, synthetic construct
  source 1..531: mol_type = other DNA
atgtctagacctggcgagaggcccttccagtgccggatctgcatgcggaacttcagcaacatgagcaacctgaccagacacacccggacacacacaggcgagaagccttttcagtgcagaatctgtatgcgcaatttctccgacagaagcgtgctgcggagacacctgagaacccacaccggcagccagaaaccattccagtgtcgcatctgtatgagaaactttagcgacccctccaatctggcccggcacaccagaacacataccggggaaaaaccctttcagtgtaggatatgcatgaggaatttttccgaccggtccagcctgaggcggcacctgaggacacatactggctcccaaaagccgttccaatgtcggatatgtatgcgcaactttagccagagcggcaccctgcacagacacacaagaacccatactggcgagaaacctttccaatgtagaatctgcatgcgaaatttttcccagcggcctaatctgaccaggcatctgaggacccacctgagaggatct

SEQ ID NO: 324 - skipped

SEQ ID NO: 325 - protein, 313 aa, synthetic construct
DALDDFDLDMLGSDALDDFDLDMLGSDALDDFDLDMLGSDALDDFDLDMLINSRSSGSPKKKRKVGSGGGSGGSGSVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLSGGGSGGSGSDLSHPPPRGHLDELTTTLESMTEDLNLDSPLTPELNEILDTFLNDECLLHAMHISTGLSIFDTSLF

SEQ ID NO: 326 - DNA, 9321 bp, synthetic construct
  source 1..9321: mol_type = other DNA
aagcttggaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgaggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggggccgccaccatgctgctgctggtcacatctctgctgctgtgcgagctgccccatcctgcctttctgctgatccctcacatggacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtactactgccagcagtactacaactaccctctgaccttcggccagggcaccaagctggaaatcaaaggcggcggaggatctggcggaggtggaagtggcggaggcggatctgaagtgcagctggttgaatcaggtggcggcctggttcaacctggcggatctctgagactgagctgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccagattcaccatcagccgggacgacagcaagaacagcctgtacctgcagatgaactccctgaaaaccgaggacaccgccgtgtattattgcgtggccggcaacagctttgcctactggggacagggaaccctggtcaccgtgtctgccacaacaacccctgctcctagacctcctacaccagctcctacaatcgccctgcagcctctgtctctgaggccagaagcttgtagaccagctgctggcggagccgtgcatacaagaggactggacttcgcctgtgatgtggccgccattctcggactgggacttgttctgggactgctgggacctctggccattctgctggctctgtatctgctgcggagggaccaaagactgcctcctgatgctcacaagcctccaggcggaggcagcttcagaacccctatccaagaggaacaggccgacgctcacagcaccctggccaagattagagtgaagttcagcagaagcgccgacgcacccgcctataagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggtagcggccagtgtaccaactacgccctgctgaaactggccggcgacgtggaatctaatcctggacctggatctggcgagggacgcgggagtctactgacgtgtggagacgtggaggaaaaccctggacctatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgtttaaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 327 - DNA, 9393 bp, synthetic construct
  source 1..9393: mol_type = other DNA
aagcttggaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgaggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggggccgccaccatgctgctgctggtcacatctctgctgctgtgcgagctgccccatcctgcctttctgctgatccctcacatggaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccaggttcaccatctccagagatgacagcaagaacagcctgtacctgcagatgaactccctgaaaaccgaggacaccgccgtgtactattgcgtggccggcaatagctttgcctactggggacagggcaccctggttacagtttctgctggcggcggaggaagcggaggcggaggatccggtggtggtggatctgacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaaatctggcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgcaggcggagccgtgcatacaagaggactggatttcgcctgcgacatctacatctgggcccctctggctggaacatgtggtgtcctgctgctgagcctggtcatcaccctgtactgcaaccaccggcggagcaagagaagcagactgctgcacagcgactacatgaacatgacccctagacggcccggacctaccagaaagcactaccagccttacgctcctcctagagacttcgccgcctaccggtccagagtgaagttcagcagatccgccgatgctcccgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggtagcggccagtgtaccaactacgccctgctgaaactggccggcgacgtggaatctaatcctggacctggatctggcgagggacgcgggagtctactgacgtgtggagacgtggaggaaaaccctggacctatggactggacctggatcctgtttctggtggccgctgccacaagagtgcacagcaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacacaagctctggcggcggaggatctggcggaggtggaagcggagttacacccgagcctatcttcagcctgatcggaggcggtagcggaggcggaggaagtggtggcggatctctgcaactgctgcctagctgggccatcacactgatctccgtgaacggcatcttcgtgatctgctgcctgacctactgcttcgcccctagatgcagagagcggcggagaaacgaacggctgagaagagaatctgtgcggcccgtttaaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 328 - DNA, 10169 bp, synthetic construct
  source 1..10169: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcttaaacgggccgcacagattctcttctcagccgttcgtttctccgccgctctctgcatctaggggcgaagcagtaggtcaggcagcagatcacgaagatgccgttcacggagatcagtgtgatggcccagctaggcagcagttgcagagatccgccaccacttcctccgcctccgctaccgcctccgatcaggctgaagataggctcgggtgtaactccgcttccacctccgccagatcctccgccgccagagcttgtgttgatgaacatctgcacgatgtgcacgaagctctgcaggaactctttgatattcttctcttccagttcctcgcactctttgcagccggactcggtcacattgccgttgctgctcaggctgttgttggccaggatgatcaggttttccacggtgtcgtggatgctggcgtcgccgctttccaggctgatcacttgcagttccagcagaaagcacttcatggcggtcactttacagctagggtgcacgtcgctctcggtgtacagtgtggcgtcgatgtgcatgctctggatcaggtcctcgatcttcttcaggtcgctgatcacgttgacccaattgctgtgcactcttgtggcagcggccaccagaaacaggatccaggtccagtccatggtggcggcacgcgtctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtaatagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgaattcccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgctgctgctggtcacatctctgctgctgtgcgagctgccccatcctgcctttctgctgatccctcacatggaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccaggttcaccatctccagagatgacagcaagaacagcctgtacctgcagatgaactccctgaaaaccgaggacaccgccgtgtactattgcgtggccggcaatagctttgcctactggggacagggcaccctggttacagtttctgctggcggcggaggaagcggaggcggaggatccggtggtggtggatctgacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaaatctggcgccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgcaggcggagccgtgcatacaagaggactggatttcgcctgcgacatctacatctgggcccctctggctggaacatgtggtgtcctgctgctgagcctggtcatcaccctgtactgcaaccaccggcggagcaagagaagcagactgctgcacagcgactacatgaacatgacccctagacggcccggacctaccagaaagcactaccagccttacgctcctcctagagacttcgccgcctaccggtccagagtgaagttcagcagatccgccgatgctcccgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggataaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 329 - DNA, 10082 bp, synthetic construct
  source 1..10082: mol_type = other DNA
aagcttgaattcgagcttgcatgcctgcaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcaataaaagagcccacaacccctcactcggcgcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgagatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggccccccctcgagtccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcttaaacgggccgcacagattctcttctcagccgttcgtttctccgccgctctctgcatctaggggcgaagcagtaggtcaggcagcagatcacgaagatgccgttcacggagatcagtgtgatggcccagctaggcagcagttgcagagatccgccaccacttcctccgcctccgctaccgcctccgatcaggctgaagataggctcgggtgtaactccgcttccacctccgccagatcctccgccgccagagcttgtgttgatgaacatctgcacgatgtgcacgaagctctgcaggaactctttgatattcttctcttccagttcctcgcactctttgcagccggactcggtcacattgccgttgctgctcaggctgttgttggccaggatgatcaggttttccacggtgtcgtggatgctggcgtcgccgctttccaggctgatcacttgcagttccagcagaaagcacttcatggcggtcactttacagctagggtgcacgtcgctctcggtgtacagtgtggcgtcgatgtgcatgctctggatcaggtcctcgatcttcttcaggtcgctgatcacgttgacccaattgctgtgcactcttgtggcagcggccaccagaaacaggatccaggtccagtccatggtggcggcacgcgtctggggagagaggtcggtgattcggtcaacgagggagccgactgccgacgtgcgctccggaggcttgcagaatgcggaacaccgcgcgggcaggaacagggcccacactaccgccccacaccccgcctcccgcaccgccccttcccggccgctgctctcggcgcgccctgctgagcagccgctattggccacagcccatcgcggtcggcgcgctgccattgctccctggcgctgtccgtctgcgagggtaatagtgagacgtgcggcttccgtttgtcacgtccggcacgccgcgaaccgcaaggaaccttcccgacttaggggcggagcaggaagcgtcgccggggggcccacaagggtagcggcgaagatccgggtgacgctgcgaacggacgtgaagaatgtgcgagacccagggtcggcgccgctgcgtttcccggaaccacgcccagagcagccgcgtccctgcgcaaacccagggctgccttggaaaaggcgcaaccccaaccccgaattcccgataaaataaaagattttatttagtctccagaaaaaggggggaatgaaagaccccacctgtaggtttggcaagctagctgcagtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaggatccgccaccatgctgctgctggtcacatctctgctgctgtgcgagctgccccatcctgcctttctgctgatccctcacatggaagtgcagctggtggaatctggcggaggactggttcaacctggcggctctctgagactgtcttgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccaggttcaccatctccagagatgacagcaagaacagcctgtacctgcagatgaactccctgaaaaccgaggacaccgccgtgtactattgcgtggccggcaatagctttgcctactggggacagggcaccctggttacagtttctgctggcggcggaggaagcggaggcggaggatccggtggtggtggatctgacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaagaccaccacaccagctcctcggccaccaactccagctccaacaattgccagccagcctctgtctctgaggcccgaagcttgtagacctgctgcaggcggagccgtgcatacaagaggactggatttcgcctgcgacatctacatctgggcccctctggctggaacatgtggtgtcttgctgctgagcctggtcatcaccaagcggggcagaaagaagctgctgtacatcttcaagcagcccttcatgcggcccgtgcagaccacacaagaggaagatggctgcagctgtcggttccccgaggaagaagaaggcggctgcgagctgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaggatccggattagtccaatttgttaaagacaggatgggctgcaggaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggagaattcgatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcaataaaagagcccacaacccctcactcggggcgccagtcctccgattgactgagtcgcccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatcgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagatcccgcggtacctcgcgaatgcatctagatccaatggcctttttggcccagacatgataagatacattgatgagtttggacaaaccacaactagaatgcagtgaaaaaaatgctttatttgtgaaatttgtgatgctattgctttatttgtaaccattataagctgcaataaacaagttgcggccgcttagccctcccacacataaccagagggcagcaattcacgaatcccaactgccgtcggctgtccatcactgtccttcactatggctttgatcccaggatgcagatcgagaagcacctgtcggcaccgtccgcaggggctcaagatgcccctgttctcatttccgatcgcgacgatacaagtcaggttgccagctgccgcagcagcagcagtgcccagcaccacgagttctgcacaaggtcccccagtaaaatgatatacattgacaccagtgaagatgcggccgtcgctagagagagctgcgctggcgacgctgtagtcttcagagatggggatgctgttgattgtagccgttgctctttcaatgagggtggattcttcttgagacaaaggcttggccatgcggccgccgctcggtgttcgaggccacacgcgtcaccttaatatgcgaagtggacctcggaccgcgccgccccgactgcatctgcgtgttcgaattcgccaatgacaagacgctgggcggggtttgtgtcatcatagaactaaagacatgcaaatatatttcttccggggggtaccggcctttttggccattggatcggatctggccaaaaaggcccttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgattgtgtatatgtgtgtatggttgtgtgtgattgtgtgtatgtatgtttgtgtgtgattgtgtgtgtgtgattgtgcatgtgtgtgtgtgtgattgtgtttatgtgtatgattgtgtgtgtgtgtgtgtgtgtgtgtgtgtgtgtgtgtgtgtgtgtgttgtgtatatatatttatggtagtgagaggcaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 330 - DNA, 351 bp, synthetic construct
  source 1..351: mol_type = other DNA
gaagtgcagctggttgaatcaggtggcggcctggttcaacctggcggatctctgagactgagctgtgccgccagcggcttcaccttcaacaagaacgccatgaactgggtccgacaggcccctggcaaaggccttgaatgggtcggacggatccggaacaagaccaacaactacgccacctactacgccgacagcgtgaaggccagattcaccatcagccgggacgacagcaagaacagcctgtacctgcagatgaactccctgaaaaccgaggacaccgccgtgtattattgcgtggccggcaacagctttgcctactggggacagggaaccctggtcaccgtgtctgcc

SEQ ID NO: 331 - DNA, 138 bp, synthetic construct
  source 1..138: mol_type = other DNA
ctgctgcctagctgggccatcacactgatctccgtgaacggcatcttcgtgatctgctgcctgacctactgcttcgcccctagatgcagagagcggcggagaaacgaacggctgagaagagaatctgtgcggcccgtt

SEQ ID NO: 332 - DNA, 45 bp, synthetic construct
  source 1..45: mol_type = other DNA
ggcggcggaggaagcggaggcggaggatccggtggtggtggatct

SEQ ID NO: 333 - DNA, 339 bp, synthetic construct
  source 1..339: mol_type = other DNA
gacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaaa

SEQ ID NO: 334 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 335 - DNA, 198 bp, synthetic construct
  source 1..198: mol_type = other DNA
gccctgagcaacagcatcatgtacttcagccacttcgtgcccgtgtttctgcccgccaagcctacaacaacccctgctcctagacctcctacaccagctcctacaatcgccagccagcctctgtctctgaggccagaagcttgtagacctgctgcaggcggagccgtgcatacaagaggactggatttcgcctgcgac

SEQ ID NO: 336 - DNA, 339 bp, synthetic construct
  source 1..339: mol_type = other DNA
gacatcgtgatgacacagagccccgatagcctggccgtgtctctgggagaaagagccaccatcaactgcaagagcagccagagcctgctgtactccagcaaccagaagaactacctggcctggtatcagcaaaagcccggccagcctcctaagctgctgatctattgggccagctccagagaaagcggcgtgcccgatagattttctggctctggcagcggcaccgacttcaccctgacaatttctagcctgcaagccgaggacgtggccgtgtattactgccagcagtactacaactaccctctgaccttcggccagggcaccaagctggaaatcaag

SEQ ID NO: 337 - DNA, 135 bp, synthetic construct
  source 1..135: mol_type = other DNA
accaccacaccagctcctcggccaccaactccagctccaacaattgccagccagcctctgtctctgaggcccgaagcttgtagacctgctgcaggcggagccgtgcatacaagaggactggatttcgcctgcgac

SEQ ID NO: 338 - DNA, 63 bp, synthetic construct
  source 1..63: mol_type = other DNA
atctacatctgggcccctctggctggaacatgtggtgtcttgctgctgagcctggtcatcacc

SEQ ID NO: 339 - DNA, 126 bp, synthetic construct
  source 1..126: mol_type = other DNA
aagcggggcagaaagaagctgctgtacatcttcaagcagcccttcatgcggcccgtgcagaccacacaagaggaagatggctgcagctgtcggttccccgaggaagaagaaggcggctgcgagctg

SEQ ID NO: 340 - DNA, 24 bp, synthetic construct
  source 1..24: mol_type = other DNA
atgcccaagaaaaagcggaaggtg

SEQ ID NO: 341 - DNA, 528 bp, synthetic construct
  source 1..528: mol_type = other DNA
tcccggcctggcgagaggcctttccagtgcagaatctgcatgcggaacttcagcagacggcacggcctggacagacacaccagaacacacacaggcgagaaacccttccagtgccggatctgtatgagaaatttcagcgaccacagcagcctgaagcggcacctgagaacccataccggcagccagaaaccatttcagtgtaggatatgcatgcgcaatttctccgtgcggcacaacctgaccagacacctgaggacacacaccggggagaagccttttcaatgtcgcatatgcatgagaaacttctctgaccactccaacctgagccgccacctcaaaacccacaccggctctcaaaagcccttccaatgtagaatatgtatgaggaactttagccagcggagcagcctcgtgcgccatctgagaactcacactggcgaaaagccgtttcaatgccgtatctgtatgcgcaactttagcgagagcggccacctgaagagacatctgcgcacacacctgagaggcagc

SEQ ID NO: 342 - DNA, 795 bp, synthetic construct
  source 1..795: mol_type = other DNA
gaggatgtcgtgtgctgccacagcatctacggaaagaagaagggcgacatcgacacctatcggtacatcggcagcagcggcacaggctgtgttgtgatcgtgggcagaatcgtgctgagcggctctggaacaagcgcccctatcacagcctacgctcagcagacaagaggcctgctgggctgcatcatcacaagcctgaccggcagagacaagaaccaggtggaaggcgaggtgcagatcgtgtctacagctacccagaccttcctggccacctgtatcaatggcgtgtgctgggccgtgtatcacggcgctggcacaagaacaatcgcctctccaaagggccccgtgatccagatgtacaccaacgtggaccaggacctcgttggctggcctgctcctcaaggcagcagaagcctgacaccttgcacctgtggctccagcgatctgtacctggtcaccagacacgccgacgtgatccctgtcagaagaagaggggattccagaggcagcctgctgagccctagacctatcagctacctgaagggcagctctggcggacctctgctttgtcctgctggacatgccgtgggcctgtttagagccgccgtgtgtacaagaggcgtggccaaagccgtggacttcatccccgtggaaaacctggaaaccaccatgcggagccccgtgttcaccgacaattctagccctccagccgtgacactgacacaccccatcaccaagatcgacagagaggtgctgtaccaagagttcgacgagatggaagagtgcagccagcac

SEQ ID NO: 343 - DNA, 942 bp, synthetic construct
  source 1..942: mol_type = other DNA
gacgctcttgatgactttgacctggatatgctcggatcagatgccctggacgatttcgatctggacatgttggggtctgatgctctcgacgacttcgatctggatatgcttggaagtgacgcgctggatgatttcgaccttgacatgctcatcaattctcgatccagtggaagcccgaaaaagaaacgcaaggtgggaagtgggggcggctccggtgggagcggtagtgtattgcctcaagctcccgcgcccgctcctgctccggcaatggtttcagctctggcacaagctccagctccagtgcctgtgctcgcccctggccctccgcaggccgtagcacctcccgcccccaaaccgacgcaagccggtgaggggactctctctgaagccttgctgcagcttcagttcgatgatgaagatctgggcgcgctcttggggaacagcacggatccggcagtatttacggacctcgcatcagttgacaatagtgaatttcaacaacttcttaaccagggaataccggttgcgccccatacgacggaacctatgctgatggagtaccctgaagctataaccagactcgtaactggcgcccaacgcccgcccgacccggctcctgcgccgctgggtgcgccgggtcttccgaatggtcttctctcaggggacgaagatttcagttccattgcggatatggacttttccgcgctcctgagtgggggtggctctggaggctctggttccgacctcagccatcctccaccgagaggacacctcgacgagctgacaaccaccctcgaaagtatgacggaagatctgaacttggattccccccttaccccagaactgaatgaaatcctcgatacgttcttgaacgatgagtgccttttgcacgccatgcatatatcaacaggtttgtctatcttcgacacgtccctcttttga

SEQ ID NO: 344 - protein, 5 aa, synthetic construct
  VARIANT 2: note = X can be G or A
GXELR

SEQ ID NO: 345 - protein, 6 aa, synthetic construct
  VARIANT 4..5: note = X can be any amino acid
DSGXXS

SEQ ID NO: 346 - protein, 4 aa, synthetic construct
WRPW

SEQ ID NO: 347 - protein, 10 aa, synthetic construct
GSGSGSGSGG

SEQ ID NO: 348 - protein, 17 aa, synthetic construct
AEAAAKEAAAKEAAAKA

SEQ ID NO: 349 - protein, 18 aa, synthetic construct
KESGSVSSEQLAQFRSLD

SEQ ID NO: 350 - protein, 14 aa, synthetic construct
EGKSSGSGSESKST

SEQ ID NO: 351 - DNA, 837 bp, synthetic construct
  source 1..837: mol_type = other DNA
taccctccagtgatcgtggaaatgaacagcagcgtggaagccatcgagggctctcatgtgtctctgctgtgtggcgccgacagcaatcctcctcctctgctgacctggatgagagatggcaccgtgctgagagaagccgtggccgaatctctgctgctggaactggaagaagtgacccctgccgaggatggcgtgtacgcttgtctggccgagaatgcctacggccaggacaatagaaccgtgggcctgtccgtgatgtacgccccttggaagcctaccgtgaacggcacaatggtggccgtggaaggcgagacagtgtccatcctgtgtagcacccagagcaaccccgatcctatcctgaccatcttcaaagagaagcagatcctgagcaccgtgatctacgagagcgaactgcagctcgaactgcccgctgtgtccccagaggatgatggcgaatattggtgcgtggcagagaaccagtacggccagagagccaccgccttcaacctgagcgtggaatttgctcccgtgctgctgctcgagagccattgtgctgccgccagagataccgtgcagtgcctgtgtgtggtcaagtctaaccccgagcctagcgtggcctttgagctgcccagcagaaacgtgaccgtgaatgagagcgagcgcgagttcgtgtacagcgagagatctggactggtgctgaccagcatcctgacactgagaggacaggctcaggcccctcctagagtgatctgcaccgccagaaatctgtacggcgccaagagcctggaactgccatttcagggcgcccacagactcatgtgggccaagattggacct

SEQ ID NO: 352 - DNA, 136 bp, synthetic construct
  source 1..136: mol_type = other DNA
acaacaacccctgctcctagacctcctacaccagctcctacaatcgccctgcagcctctgtctctgaggccagaagcttgtagaccagctgctggcggagccgtgcatacaagaggactggacttcgcctgtgatg

SEQ ID NO: 353 - protein, 66 aa, synthetic construct
ALSNSIMYFSHFVPVFLPAKPTTTPAPRPPTPAPTIASQPLSLRPEACRPAAGGAVHTRGLDFACD

SEQ ID NO: 354 - protein, 176 aa, synthetic construct
SRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 355 - protein, 229 aa, synthetic construct
MDWTWILFLVAAATRVHSYPYDVPDYAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGVTPEPIFSLIGGGSGGGGSGGGSLQLLPSWAITLISVNGIFVICCLTYCFAPRCRERRRNERLRRESVRPV

SEQ ID NO: 356 - protein, 228 aa, synthetic construct
MDWTWILFLVAAATRVHSYPYDVPDYAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSPRAEALKGGSGGGGSGGGGSGGGGSGGGGSGGGSLQLLPSWAITLISVNGIFVICCLTYCFAPRCRERRRNERLRRESVRPV

SEQ ID NO: 357 - protein, 134 aa, synthetic construct
METDTLLLWVLLLWVPGSTGNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 358 - protein, 219 aa, synthetic construct
MDWTWILFLVAAATRVHSYPYDVPDYAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGGGGSGGGGSGGGSLQLLPSWAITLISVNGIFVICCLTYCFAPRCRERRRNERLRRESVRPV

SEQ ID NO: 359 - protein, 155 aa, synthetic construct
MERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 360 - protein, 133 aa, synthetic construct [sequence omitted]

SEQ ID NO: 361 - protein, 300 aa, synthetic construct
MDWTWILFLVAAATRVHSITCPPPMSVEHADIWVKSYSLYSRERYICNSGFKRKAGTSSLTECVLNKATNVAHWTTPSLKCIRSGGSGGGGSGGGSGGGGSLQNWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTSSGGGGSGGGGSGVTPEPIFSLIGGGSGGGGSGGGSLQLLPSWAITLISVNGIFVICCLTYCFAPRCRERRRNERLRRESVRPV

SEQ ID NO: 362 - protein, 500 aa, synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 363 - protein, 495 aa, synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 364 - protein, 495 aa, synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 365 - protein, 620 aa, synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 366 - protein, 516 aa, synthetic construct
MALPVTALLLPLALLLHAARPEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGSTSGSGKPGSGEGSTKGDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGKPIPNPLLGLDSTNGAATTTPAPRPPTPAPTIALQPLSLRPEACRPAAGGAVHTRGLDFACDVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 367 - DNA, 687 bp, synthetic construct
  source 1..687: mol_type = other DNA
atggactggacttggatactctttctggtcgctgccgccacacgggtgcactcttatccatatgatgttccagattatgctggcggaggcggttctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtg

SEQ ID NO: 368 - DNA, 684 bp, synthetic construct
  source 1..684: mol_type = other DNA
atggactggacttggatactctttctggtcgctgccgccacacgggtgcactcttatccatatgatgttccagattatgctggcggaggcggttctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcacccagagccgaggctctgaaaggcggatcaggcggcggtggtagtggaggcggaggctcaggcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtg

SEQ ID NO: 369 - DNA, 402 bp, synthetic construct
  source 1..402: mol_type = other DNA
atggaaaccgacacactgctgctgtgggtgctgcttctttgggtgcccggctctacaggcaactgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggctaacaacagcctgagcagcaacggcaatgtgaccgagtccggctgcaaagagtgcgaggaactggaagagaagaatatcaaagagttcctccagagcttcgtgcacatcgtgcagatgttcatcaacaccagc

SEQ ID NO: 370 - DNA, 657 bp, synthetic construct
  source 1..657: mol_type = other DNA
atggactggacttggatactctttctggtcgctgccgccacacgggtgcactcttatccatatgatgttccagattatgctggcggaggcggttctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtg

SEQ ID NO: 371 - DNA, 465 bp, synthetic construct
  source 1..465: mol_type = other DNA
atggaacggatcgtgatctgcctgatggtcatcttcctgggcaccctggtgcacaagagcagctctcagggccaagaccggcacatgatccggatgagacagctgatcgacatcgtggaccagctgaagaactacgtgaacgacctggtgcctgagtttctgcccgctcctgaggacgtggaaacaaactgcgagtggagcgccttcagctgcttccagaaggcccagctgaaaagcgccaacaccggcaacaacgagcggatcatcaacgtgtccatcaagaagctgaagcggaagcctcctagcaccaacgccggaagaaggcagaagcacagactgacctgtcctagctgcgacagctacgagaagaagcctccaaaagagtttctcgagcggttcaagagcctgctgcagaagatgatccaccagcacctgtccagcaggacacacggcagcgaggattct

SEQ ID NO: 372 - DNA, 900 bp, synthetic construct
  source 1..900: mol_type = other DNA
aacgggccgcacagattctcttctcagccgttcgtttctccgccgctctctgcatctaggggcgaagcagtaggtcaggcagcagatcacgaagatgccgttcacggagatcagtgtgatggcccagctaggcagcagttgcagagatccgccaccacttcctccgcctccgctaccgcctccgatcaggctgaagataggctcgggtgtaactccgcttccacctccgccagatcctccgccgccagagcttgtgttgatgaacatctgcacgatgtgcacgaagctctgcaggaactctttgatattcttctcttccagttcctcgcactctttgcagccggactcggtcacattgccgttgctgctcaggctgtcgttggccaggatgatcaggttttccacggtgtcgtggatgctggcgtcgccgctttccaggctgatcacttgcagttccagcagaaagcacttcatggcggtcactttacagctagggtgcacgtcgctctcggtgtacagtgtggcgtcgatgtgcatgctctggatcaggtcctcgatcttcttcaggtcgctgatcacgttgacccaattctgcagagatcctccgcctccgcttccaccgccagaacctccgccgccagatccgccgcttctgatacacttcaggctaggtgtggtccagtgggccacattggtggccttgttcagcacacactcggtcaggctgctggtgccggcctttctcttgaagccgctgttgcagatgtaccgctctctgctgtacaggctgtagctcttgacccagatgtcggcgtgttccacgctcataggtggaggacaggtgatgctgtgcactcttgtggcagcggccaccagaaacaggatccaggtccagtccat

SEQ ID NO: 373 - DNA, 1500 bp, synthetic construct
  source 1..1500: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 374 - DNA, 1485 bp, synthetic construct
  source 1..1485: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgtggccgccattcttggactgggacttgttctgggactgctgggacctctggccattctgctggctctgtacctgctgagaagggaccagagactgcctcctgacgctcacaaacctccaggcggaggcagcttcagaacccctatccaagaggaacaggctgacgcccacagcaccctggccaagatcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 375 - DNA, 1485 bp, synthetic construct
  source 1..1485: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatatctacatctgggcccctctggctggaacatgcggagtgttgctgctgagcctggtcatcaccaagcggggcagaaagaagctgctgtacatcttcaagcagcccttcatgcggcccgtgcagaccacacaagaggaagatggctgctcctgcagattccccgaggaagaagaaggcggctgcgagctgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 376 - DNA, 1860 bp, synthetic construct
  source 1..1860: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctgaacaaaaactcatctcagaagaagatctgaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcctggcgccgcaagcggaaagagaagcagtctgagacaagccccaaagagttcctgaccatctacgaggacgtgaaggacctgaaaacccggcggaaccacgagcaagagcagacctttcctggcggcggaagcaccatctacagcatgatccagagccagtctagcgcccctaccagccaagagcctgcctacacactgtactccctgatccagcctagcagaaagagcggcagccggaagagaaatcacagccccagcttcaacagcacgatctacgaagtgatcggcaagagccagccaaaggctcagaaccctgccaggctgagccggaaagagctggaaaacttcgacgtgtacagcagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 377 - DNA, 1548 bp, synthetic construct
  source 1..1548: mol_type = other DNA
atggccttaccagtgaccgccttgctcctgccgctggccttgctgctccacgccgccaggccggaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctggcagcacaagcggctctggaaaacctggatctggcgagggctctaccaagggcgacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaaggggaagcctatcccgaaccctctgttgggtctcgatagtaccaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatgttatagggatcctggtggctgtcatactcctcttgctcctcttgttgctgctttttttgatattgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcac

SEQ ID NO: 378 - DNA, 729 bp, synthetic construct
  source 1..729: mol_type = other DNA
gaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctggcagcacaagcggctctggaaaacctggatctggcgagggctctaccaagggcgacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaag

SEQ ID NO: 379 - protein, 243 aa, synthetic construct
EVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGSTSGSGKPGSGEGSTKGDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIK

SEQ ID NO: 380 - DNA, 720 bp, synthetic construct
  source 1..720: mol_type = other DNA
gacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggatctggaagcggcggttctggatctggtggaagcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctct

SEQ ID NO: 381 - protein, 240 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 382 - DNA, 60 bp, synthetic construct
  source 1..60: mol_type = other DNA
atcgtggtgggcgtggtgtgcaccctgctggtggccctgctgatggccgccctgtacctg

SEQ ID NO: 383 - protein, 20 aa, synthetic construct
IVVGVVCTLLVALLMAALYL

SEQ ID NO: 384 - DNA, 333 bp, synthetic construct
  source 1..333: mol_type = other DNA
gtgagaatcagacagaagaaggcccagggcagcaccagcagcaccagactgcacgagcccgagaagaacgccagagagatcacccaggacaccaacgacatcacctacgccgacctgaacctgcccaagggcaagaagcccgccccccaggccgccgagcccaacaaccacaccgagtacgccagcatccagaccagcccccagcccgccagcgaggacaccctgacctacgccgacctggacatggtgcacctgaacagaacccccaagcagcccgcccccaagcccgagcccagcttcagcgagtacgccagcgtgcaggtgcccagaaag

SEQ ID NO: 385 - protein, 111 aa, synthetic construct
VRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 386 - DNA, 399 bp, synthetic construct
  source 1..399: mol_type = other DNA
cagggccaagaccggcacatgatccggatgagacagctgatcgacatcgtggaccagctgaagaactacgtgaacgacctggtgcctgagtttctgcccgctcctgaggacgtggaaacaaactgcgagtggagcgccttcagctgcttccagaaggcccagctgaaaagcgccaacaccggcaacaacgagcggatcatcaacgtgtccatcaagaagctgaagcggaagcctcctagcaccaacgccggaagaaggcagaagcacagactgacctgtcctagctgcgacagctacgagaagaagcctccaaaagagtttctcgagcggttcaagagcctgctgcagaagatgatccaccagcacctgtccagcaggacacacggcagcgaggattct

SEQ ID NO: 387 - protein, 168 aa, synthetic construct
LRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 388 - DNA, 504 bp, synthetic construct
  source 1..504: mol_type = other DNA
ttgcgccacagacggcagggaaagcactggactagtacgcagaggaaagcggacttccagcatcccgcaggagccgtggggcctgaacccactgatcgcggccttcaatggaggtctagcccggcggcagacgcacaagaggaaaacttgtacgcagccgttaagcacacccaaccggaggacggcgttgagatggatacccgctcccctcacgatgaagaccctcaagcagtcacttacgcggaagtaaagcatagccgccccagacgggaaatggctagcccgccgtccccccttagcggggaatttctggacactaaagataggcaggcggaagaggaccgccaaatggatacagaggcggcggcaagtgaagcacctcaagacgttacttacgctcaacttcacagccttaccctcaggcgagaagcgactgaaccacccccttcccaagaagggccaagcccagcggttccttctatctatgctactcttgctattcac

SEQ ID NO: 389 - protein, 21 aa, synthetic construct
VIGILVAVILLLLLLLLLFLI

SEQ ID NO: 390 - DNA, 63 bp, synthetic construct
  source 1..63: mol_type = other DNA
gttatagggatcctggtggctgtcatactcctcttgctcctcttgttgctgctttttttgata

SEQ ID NO: 391 - protein, 325 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGSGGGGSGGGSGGGGSLQITCPPPMSVEHADIWVKSYSLYSRERYICNSGFKRKAGTSSLTECVLNKATNVAHWTTPSLKCIRAAAIEVMYPPPYLDNEKSNGTIIHVKGKHLCPSPLFPGPSKPFWVLVVVGGVLACYSLLVTVAFIIFWVKSRQTPPLASVEMEAMEALPVTWGTSSRDEDLENCSHHL

SEQ ID NO: 392 - DNA, 978 bp, synthetic construct
  source 1..978: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagagcgacgtgcaccctagctgtaaagtgaccgccatgaagtgctttctgctggaactgcaagtgatcagcctggaaagcggcgacgccagcatccacgacaccgtggaaaacctgatcatcctggccaacaacagcctgagcagcaacggcaatgtgaccgagtccggctgcaaagagtgcgaggaactggaagagaagaatatcaaagagttcctgcagagcttcgtgcacatcgtgcagatgttcatcaacaccagcagcggcggatctggcggcggaggttctggcggtggaagcggaggcggaggatctctccagatcacatgccctccacctatgagcgtggaacacgccgacatctgggtcaagagctacagcctgtacagcagagagcggtacatctgcaacagcggcttcaagagaaaggccggcacaagcagcctgaccgagtgcgtgctgaacaaggccacaaatgtggcccactggaccacacctagcctgaagtgcatcagagcagcagctatcgaggtgatgtatcctccgccctacctggataatgaaaagagtaatgggactatcattcatgtaaaagggaagcatctttgtccttctccccttttccccggtccgtctaaacctttctgggtgctcgtggttgttggcggagtgctggcctgttactctctgctggtcaccgtggccttcatcatcttttgggtcaagtccagacagacacctcctctggccagcgtggaaatggaagccatggaagctctgcctgtgacctggggcaccagctccagagatgaggacctggaaaactgctcccaccacctgtaa

SEQ ID NO: 393 - DNA, 456 bp, synthetic construct
  source 1..456: mol_type = other DNA
gactgtgatatcgagggcaaagacggcaagcagtacgagagcgtgctgatggtgtccatcgaccagctgctggacagcatgaaggaaatcggcagcaactgcctgaacaacgagttcaacttcttcaagcggcacatctgcgacgccaacaaagaaggcatgttcctgttcagagccgccagaaagctgcggcagttcctgaagatgaacagcaccggcgacttcgacctgcatctgctgaaagtgtctgagggcaccaccatcctgctgaattgcaccggccaagtgaagggcagaaagcctgctgctctgggagaagcccagcctaccaagagcctggaagagaacaagtccctgaaagagcagaagaagctgaacgacctctgcttcctgaagcggctgctgcaagagatcaagacctgctggaacaagatcctgatgggcaccaaagagcac

SEQ ID NO: 394 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 395 - protein, 18 aa, synthetic construct
GSTSGSGKPGSGEGSTKG

SEQ ID NO: 396 - protein, 10 aa, synthetic construct
GGSGSGGSGS

SEQ ID NO: 397 - protein, 12 aa, synthetic construct
SAGSGSGASGSG

SEQ ID NO: 398 - DNA, 132 bp, synthetic construct
  source 1..132: mol_type = other DNA
ggttctggccagtgcacaaattatgcactgctgaagctcgccggggatgtcgagagtaacccaggacctggaagcggagaaggtcgtggtagtctactaacgtgtggtgatgtagaagaaaatcctggacct

SEQ ID NO: 399 - DNA, 132 bp, synthetic construct; identical to SEQ ID NO: 282
  source 1..132: mol_type = other DNA

SEQ ID NO: 400 - DNA, 132 bp, synthetic construct
  source 1..132: mol_type = other DNA
ggctctggccagtgcaccaactacgccctgcttaaactggccggcgacgtggaatccaatcctggacctggatctggcgagggacgcgggagtctactgacgtgtggagacgtggaggaaaaccctggacct

SEQ ID NO: 401 - protein, 44 aa, synthetic construct; identical to SEQ ID NO: 281

SEQ ID NO: 402 - protein, 44 aa, synthetic construct; identical to SEQ ID NO: 281

SEQ ID NO: 403 - protein, 44 aa, synthetic construct; identical to SEQ ID NO: 281

SEQ ID NO: 404 - DNA, 54 bp, synthetic construct
  source 1..54: mol_type = other DNA
ggcagcacaagcggctctggaaaacctggatctggcgagggctctaccaagggc

SEQ ID NO: 405 - protein, 1478 aa, synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPGSGEGRGSLLTCGDVEENPGPMALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRKGSGQCTNYALLKLAGDVESNPGPGSGEGRGSLLTCGDVEENPGPMDWTWILFLVAAATRVHSYPYDVPDYAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGVTPEPIFSLIGGGSGGGGSGGGSLQLLPSWAITLISVNGIFVICCLTYCFAPRCRERRRNERLRRESVRPVRRKRGSGQCTNYALLKLAGDVESNPGPGSGEGRGSLLTCGDVEENPGPMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 406 - protein, 1474 aa, synthetic construct
MDWTWILFLVAAATRVHSYPYDVPDYAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGVTPEPIFSLIGGGSGGGGSGGGSLQLLPSWAITLISVNGIFVICCLTYCFAPRCRERRRNERLRRESVRPVGSGQCTNYALLKLAGDVESNPGPGSGEGRGSLLTCGDVEENPGPMALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPGSGEGRGSLLTCGDVEENPGPMALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRKGSGQCTNYALLKLAGDVESNPGPGSGEGRGSLLTCGDVEENPGPMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 407 - protein, 1478 aa, synthetic construct
MDWTWILFLVAAATRVHSYPYDVPDYAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGVTPEPIFSLIGGGSGGGGSGGGSLQLLPSWAITLISVNGIFVICCLTYCFAPRCRERRRNERLRRESVRPVRRKRGSGQCTNYALLKLAGDVESNPGPGSGEGRGSLLTCGDVEENPGPMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGSGQCTNYALLKLAGDVESNPGPGSGEGRGSLLTCGDVEENPGPMALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPGSGEGRGSLLTCGDVEENPGPMALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 408 - protein, 1478 aa, synthetic construct
MERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRRKRGSGQCTNYALLKLAGDVESNPGPGSGEGRGSLLTCGDVEENPGPMDWTWILFLVAAATRVHSYPYDVPDYAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGVTPEPIFSLIGGGSGGGGSGGGSLQLLPSWAITLISVNGIFVICCLTYCFAPRCRERRRNERLRRESVRPVGSGQCTNYALLKLAGDVESNPGPGSGEGRGSLLTCGDVEENPGPMALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSEQKLISEEDLNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPGSGEGRGSLLTCGDVEENPGPMALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 409 - protein, 1357 aa, synthetic construct
MERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRRKRGSGQCTNYALLKLAGDVESNPGPGSGEGRGSLLTCGDVEENPGPMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGQCTNYALLKLAGDVESNPGPGSGEGRGSLLTCGDVEENPGPMALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGGGSGGGGSGGGGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGQCTNYALLKLAGDVESNPGPGSGEGRGSLLTCGDVEENPGPMALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSNGAATTTPAPRPPTPAPTIALQPLSLRPEACRPAAGGAVHTRGLDFACDIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 410 - protein, 132 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 411 - DNA, 396 bp, synthetic construct
  source 1..396: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctca

SEQ ID NO: 412 - DNA, 465 bp, synthetic construct
  source 1..465: mol_type = other DNA
atggaacggatcgtgatctgcctgatggtcatcttcctgggcaccctggtgcacaagagcagctctcagggccaagaccggcacatgatccggatgagacagctgatcgacatcgtggaccagctgaagaactacgtgaacgacctggtgcctgagttcctgcctgctcctgaggacgtggaaacaaactgcgagtggagcgccttcagctgcttccagaaggcccagctgaaaagcgccaacaccggcaacaacgagcggatcatcaacgtgtccatcaagaagctgaagcggaagcctcctagcaccaacgccggaagaaggcagaagcacagactgacctgtcctagctgcgacagctacgagaagaagcctccaaaagagttcctggaacggttcaagagcctgctgcagaagatgatccaccagcacctgagcagcagaacccacggcagcgaggactcc

SEQ ID NO: 413 - protein, 159 aa, synthetic construct
MERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRRKR

SEQ ID NO: 414 - DNA, 477 bp, synthetic construct
  source 1..477: mol_type = other DNA
atggaacggatcgtgatctgcctgatggtcatcttcctgggcaccctggtgcacaagagcagctctcagggccaagaccggcacatgatccggatgagacagctgatcgacatcgtggaccagctgaagaactacgtgaacgacctggtgcctgagttcctgcctgctcctgaggacgtggaaacaaactgcgagtggagcgccttcagctgcttccagaaggcccagctgaaaagcgccaacaccggcaacaacgagcggatcatcaacgtgtccatcaagaagctgaagcggaagcctcctagcaccaacgccggaagaaggcagaagcacagactgacctgtcctagctgcgacagctacgagaagaagcctccaaaagagttcctggaacggttcaagagcctgctgcagaagatgatccaccagcacctgagcagcagaacccacggcagcgaggactccagaagaaaacgc

SEQ ID NO: 415 - protein, 233 aa, synthetic construct
MDWTWILFLVAAATRVHSYPYDVPDYAGGGGSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGVTPEPIFSLIGGGSGGGGSGGGSLQLLPSWAITLISVNGIFVICCLTYCFAPRCRERRRNERLRRESVRPVRRKR

SEQ ID NO: 416 - DNA, 699 bp, synthetic construct
  source 1..699: mol_type = other DNA
atggactggacttggatactctttctggtcgctgccgccacacgggtgcactcttatccatatgatgttccagattatgctggcggaggcggttctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagagaagaacatcaaagagtttctgcagagcttcgtccacatcgtgcagatgttcatcaacacctcatcaggcggcggtggtagtggaggcggaggctcaggcgtgacccctgagcctatcttcagcctgatcggcggaggttccggaggtggcggttccggcggaggatctcttcaattgctgcctagctgggccatcacactgatctccgtgaacggcatcttcgtgatctgctgcctgacctactgcttcgcccctagatgcagagagcggagaagaaacgagcggctgagaagagaaagcgtgcggcctgtgagaagaaaacgc

SEQ ID NO: 417 - protein, 490 aa, synthetic construct
MALPVTALLLPLALLLHAARPDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKGGGGSGGGGSGGGGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSNGAATTTPAPRPPTPAPTIALQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 418 - DNA, 1470 bp, synthetic construct
  source 1..1470: mol_type = other DNA
atggccctgcctgtgacagctctgctgctgcctctggccctgctgctgcatgctgctagacctgacatccagctgacacagagccctagcagcctgtctgcctctgtgggcgacagagtgaccatcacatgcaaggcctctcaggacgtgggcacaagcgtggcatggtatcagcagaagcctggcaaggcccctaagctgctgatctactggaccagcaccagacacacaggcgtgcccagcagattttctggcagcggctctggcaccgacttcaccttcaccataagcagcctgcagcctgaggatatcgccacctactactgccagcagtacagcctgtacagaagcttcggccagggcaccaaggtggaaatcaaaggcggaggtggaagcggcggtggtggatctggtggaggcggatctgaggtgcagctggtggaatctggtggcggagttgtgcagcctggcagatctctgagactgagctgtagcgccagcggcttcgatttcaccacctactggatgagctgggtccgacaggcccctggcaaaggactggaatggatcggcgagattcaccccgacagcagcaccatcaattacgcccctagcctgaaggaccggttcaccatctccagagacaacgccaagaataccctgttcctgcagatggacagcctccggcctgaagataccggcgtgtacttttgcgccagcctgtatttcggcttcccttggtttgcctactggggccagggaacacctgtgaccgttagctctaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 419 - protein, 458 aa, synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSGKPIPNPLLGLDSTNGAATTTPAPRPPTPAPTIALQPLSLRPEACRPAAGGAVHTRGLDFACDIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 420 - DNA, 1374 bp, synthetic construct
  source 1..1374: mol_type = other DNA
atggccttaccagtgaccgccttgctcctgccgctggccttgctgctccacgccgccaggccggacatccagatgacacagtctccagccagcctgtctgcctctgtgggagagacagtgaccatgacctgtcgggccagcgagaacatctacagctacctggcctggtatcagcagaagcagggcaagtctcctcagctgctggtgttcaacgccgagacactgcctgaaggcgtgcccagcagattttctggaacaggcagcggcacccacttcagcctgagaatcaatagcctgcagcctgaggacttcggcagctactactgccagcaccactacgtgatcccttggacctttggcggaggcaccaagctggaaatcaagggcagcacaagcggctctggaaaacctggatctggcgagggctctaccaagggcgaggtgcagatggttgagtctggcggcgatctggttaagcctggcggaagcctgaagctgtcttgtgccgccagcggcttcaccttcagcaatagcggcatgagctgggtccgacagacccctgacaagagactggaatgggtcgccagcatctctgacggcggcctgtacacacactaccccgattctgtgaagggcagattcaccatcagcagagacaacggcaagagcaccctgtacctgcagatgagcagcctgagaagcgaggacaccgccatctactactgcgccagacagggcgtcagacccttcttcgattattggggccagggcaccacactgaccgtgtcatctgggaagcctatcccgaaccctctgttgggtctcgatagtaccaatggggccgcaaccacgacgccagcgccgcgaccaccaacaccggcgcccaccatcgcgttgcagcccctgtccctgcgcccagaggcgtgccggccagcggcggggggcgcagtgcacacgagggggctggacttcgcctgtgatatcgtggtgggcgtggtgtgcaccctgctggtggccctgctgatggccgccctgtacctggtgagaatcagacagaagaaggcccagggcagcaccagcagcaccagactgcacgagcccgagaagaacgccagagagatcacccaggacaccaacgacatcacctacgccgacctgaacctgcccaagggcaagaagcccgccccccaggccgccgagcccaacaaccacaccgagtacgccagcatccagaccagcccccagcccgccagcgaggacaccctgacctacgccgacctggacatggtgcacctgaacagaacccccaagcagcccgcccccaagcccgagcccagcttcagcgagtacgccagcgtgcaggtgcccagaaag

SEQ ID NO: 421 - protein, 444 aa, synthetic construct
MALPVTALLLPLALLLHAARPDIQMTQSPASLSASVGETVTMTCRASENIYSYLAWYQQKQGKSPQLLVFNAETLPEGVPSRFSGTGSGTHFSLRINSLQPEDFGSYYCQHHYVIPWTFGGGTKLEIKGSTSGSGKPGSGEGSTKGEVQMVESGGDLVKPGGSLKLSCAASGFTFSNSGMSWVRQTPDKRLEWVASISDGGLYTHYPDSVKGRFTISRDNGKSTLYLQMSSLRSEDTAIYYCARQGVRPFFDYWGQGTTLTVSSNGAATTTPAPRPPTPAPTIALQPLSLRPEACRPAAGGAVHTRGLDFACDIVVGVVCTLLVALLMAALYLVRIRQKKAQGSTSSTRLHEPEKNAREITQDTNDITYADLNLPKGKKPAPQAAEPNNHTEYASIQTSPQPASEDTLTYADLDMVHLNRTPKQPAPKPEPSFSEYASVQVPRK

SEQ ID NO: 422 - DNA, 1332 bp, synthetic construct; identical to SEQ ID NO: 307
  source 1..1332: mol_type = other DNA

SEQ ID NO: 423 - protein, 120 aa, synthetic construct
QVQLQQSGAELVRPGTSVKVSCKASGYAFTNNLIEWVKQRPGQGLEWIGVINPGSGDTNYNEKFKGKATLTADKSSNTAYMQLSSLTSDDSAVYFCARGDYYGGFAVDYWGQGTSVTVSS

SEQ ID NO: 424 - protein, 106 aa, synthetic construct
DIQMTQTTSSLSASLGDRVTISCRTSQDIGNYLNWYQQKPDGTVKLLIYYTSRLHSGVPSRFSGSGSGTDYSLTISNLEQEDIATYFCQQGKSLPRTFGGGTKLEI

SEQ ID NO: 425 - protein, 119 aa, synthetic construct
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 426 - protein, 106 aa, synthetic construct
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIK

SEQ ID NO: 427 - protein, 448 aa, synthetic construct
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLS

SEQ ID NO: 428 - protein, 214 aa, synthetic construct
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYAEVTHQGLSSPVTKSFNRGEC

SEQ ID NO: 429 - protein, 448 aa, synthetic construct
EVQLQESGPGLVKPGGSLSLSCAASGFVFSSYDMSWVRQTPERGLEWVAYISSGGGITYAPSTVKGRFTVSRDNAKNTLYLQMNSLTSEDTAVYYCAAHYFGSSGPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 430 - protein, 214 aa, synthetic construct
DIQMTQSPASLSASVGDRVTITCRASENIFSYLAWYQQKPGKSPKLLVYNTRTLAEGVPSRFSGSGSGTDFSLTISSLQPEDFATYYCQHHYGTPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 431 - protein, 118 aa, synthetic construct
QLQESGPGLVRPSQTLSLTCTVSGFTISSGYSWHWVRQPPGRGLEWIGYIQYSGITNYNPSLKSRVTMLVDTSKNQFSLRLSSVTAADTAVYYCAREDYDYHWYFDVWGQGSLVTVSS

SEQ ID NO: 432 - protein, 110 aa, synthetic construct
GVHSDIQMTQSPSSLSASVGDRVTITCSTSSSVSYMHWYQQKPGKAPKLLIYSTSNLASGVPSRFSGSGSGTDFTFTISSLQPEDIATYYCHQWSSYPTFGQGTKVEIKR

SEQ ID NO: 433 - protein, 129 aa, synthetic construct
MDFQVQIFSFLLISASVIMSRGQTVLSQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIKR

SEQ ID NO: 434 - protein, 112 aa, synthetic construct
MDFQVQIFSFLLISASVIMSRGQTVLSQSPAILSASPGEKVTMTCRASSSVTYIHWYQQKPGSSPARFSGSGTSYSLTISRVEAEDAATYYCQHWSSKPPTFGGGTKLEIKR

SEQ ID NO: 435 - protein, 145 aa, synthetic construct
ETVIKYLLPTAAAGLLLLAAQPAMAQVKLQQSGAELVRSGTSVKLSCTASGFNIKDSYMHWLRQGPEQGLEWIGWIDPENGDTEYAPKFQGKATFTTDTSSNTAYLQLSSLTSEDTAVYYCNEGTPTGPYYFDYWGQGTTVTVSS

SEQ ID NO: 436 - protein, 109 aa, synthetic construct
ENVLTQSPAIMSASPGEKVTITCSASSSVSYMHWFQQKPGTSPKLWIYSTSNLASGVPARFSGSGSGTSYSLTISRMEAEDAATYYCQQRSSYPLTFGAGTKLELKRAA

SEQ ID NO: 437 - protein, 120 aa, synthetic construct
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSS

SEQ ID NO: 438 - protein, 106 aa, synthetic construct
ENVLTQSPSSMSVSVGDRVTIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 439 - protein, 121 aa, synthetic construct
EVKLVESGGGLVQPGGSLRLSCSISGFTFTDYYMNWVRQSPGKALEWLGFIRNKVNGDTTEYSASVKGRFTISRDISQSILYLQMNTLRTEDSATYYCARDKGIAYYFDYWGQGTTLTVSS

SEQ ID NO: 440 - protein, 106 aa, synthetic construct
QIVLSQSPAILFASPGEKVTMTCRASSSVSYIHWYQQKPGSSPKPWIHGTSNLASGVPARFSGSGSGTSYSLTISRMEAEDAATYYCQQWSSNLSTFGGGTKLEIK

SEQ ID NO: 441 - protein, 443 aa, synthetic construct
QVTLRESGPALVKPTQTLTLTCTFSGFSLSTYGIGVGWIRQPPGKALEWLAHIWWNDNKYYSTSLKTRLTISKDTSKNQVVLTMTNMDPVDTATYYCARISLPYFDYWGQGTTLTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 442 - protein, 214 aa, synthetic construct
DIQLTQSPSFLSASVGDRVTITCKASQNVGTAVAWYQQKPGKAPKLLIYSASNRYTGVPSRFSGSGSGTEFTLTISSLQPEDFATYYCQQYSSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 443 - protein, 6 aa, synthetic construct
YBTATA

SEQ ID NO: 444 - protein, 5 aa, synthetic construct
GGGGS

SEQ ID NO: 445 - protein, 5 aa, synthetic construct
  VARIANT 5: note = X can be any amino acid
LGHDX

(19) United States
(12) Patent Application Publication
(10) Pub. No.: US 2026/0144818 A1
(43) Pub. Date: May 28, 2026

(54) ARMED CHIMERIC RECEPTORS AND METHODS OF USE THEREOF

(71) Applicant: Senti Biosciences, Inc., South San Francisco, CA (US)

(72) Inventors: Alba Gonzalez-Junca, San Francisco, CA (US); Nicholas Frankel, San Francisco, CA (US)

(21) Appl. No.: 19/172,467

(22) Filed: Apr. 7, 2025

Related U.S. Application Data
(63) Continuation of application No. PCT/US2023/076287, filed on Oct. 6, 2023.
(60) Provisional application No. 63/378,846, filed on Oct. 7, 2022.
(60) Provisional application No. 63/382,477, filed on Nov. 4, 2022.
(60) Provisional application No. 63/382,646, filed on Nov. 7, 2022.

(51) Int. Cl. A61K 35/17; A61K 40/11; A61K 40/31; A61K 40/42; A61P 35/00; C07K 14/54; C07K 14/705; C07K 14/725; C07K 16/28; C07K 16/30; C12N 5/0783; C12N 15/86

(52) U.S. Cl. CPC A61K 35/17; A61K 40/11; A61K 40/31; A61K 40/421; A61P 35/00; C07K 14/5434; C07K 14/5443; C07K 14/7051; C07K 14/70517; C07K 14/70521; C07K 14/70578; C07K 16/2803; C07K 16/303; C12N 5/0646; C12N 15/86; A61K 2239/13; A61K 2239/21; C07K 2317/35; C07K 2317/53; C07K 2319/02; C07K 2319/03; C07K 2319/50; C12N 2740/15043; C12N 2840/20

(57) ABSTRACT

Described herein are immunoresponsive cells engineered to express cytokines and chimeric receptors. Also described herein are nucleic acids, cells, and methods directed to the same.

### CROSS REFERENCE TO RELATED APPLICATIONS

This application claims the benefit of and priority to U.S. Provisional Application No. 63/378,846, filed on Oct. 7, 2022; U.S. Provisional Application No. 63/382,477, filed on Nov. 4, 2022; and U.S. Provisional Application No. 63/382,646, filed on Nov. 7, 2022, the disclosures of each of which are hereby incorporated by reference in their entireties for all purposes.

### SEQUENCE LISTING

The instant application contains a Sequence Listing which has been submitted herewith and is hereby incorporated by reference in its entirety. Said XML copy, created on Aug. 1, 2025, is named SENTI1390-3_ST26.xml, and is 625,954 bytes in size.

### BACKGROUND

Cell-based therapy platforms provide promising avenues for treating a variety of diseases. One such promising platform is CAR-T based therapies in the treatment of cancer. Given their promise, improvements in cell-based therapies are needed. An active area of exploration is engineering cell-based therapies to produce and/or secrete effector molecules such as cytokines, a process referred to as armoring, that enhance the cell-based therapy. For example, unarmored CAR-T therapies have poor efficacy in solid tumors and armoring can impact the entire cancer immunity cycle and boost the activity of CAR-T. However, uncontrolled or unregulated armoring strategies can have negative impacts on treatment, such as off-target effects and toxicity in subjects. Thus, additional methods of controlling and regulating the armoring of cell-based therapies, such as regulating production and/or secretion of payload effector molecules, are required.

### SUMMARY

Provided herein, in some embodiments, is a cell-based therapy platform involving regulated armoring of the cell-based therapy, such as regulated secretion of payload effector molecules. Also provided herein, in some embodiments, is a combinatorial cell-based immunotherapy involving regulated armoring for the targeted treatment of cancer, such as ovarian cancer, breast cancer, colon cancer, lung cancer, and pancreatic cancer.

The therapy provided herein, however, can limit systemic toxicity of armoring. For example, the immunotherapy provided herein can be tumor-specific and effective while limiting systemic toxicity and/or other off-target effects due to armoring. These therapies deliver proteins of interest, such as immunomodulatory effector molecules, in a regulated manner, including regulation of secretion kinetics, cell state specificity, and cell or tissue specificity. The design of the delivery vehicle is optimized to improve overall function in cell-based therapies, such as cancer therapy, including, but not limited to, optimization of the membrane-cleavage sites, promoters, linkers, signal peptides, delivery methods, combination, regulation, and order of the immunomodulatory effector molecules.

Non-limiting examples of effector molecules encompassed by the present disclosure include cytokines, antibodies, chemokines, nucleotides, peptides, enzymes, and oncolytic viruses. For example, cells may be engineered to express and secrete in a regulated manner at least one, two, three or more of the following effector molecules: IL12, IL16, IFN-β, IFN-γ, IL2, IL15, IL7, IL36γ, IL18, IL1β, IL21, OX40-ligand, CD40L, anti-PD-1 antibodies, anti-PD-L1 antibodies, anti-CTLA-4 antibodies, anti-TGFβ antibodies, anti-TNFR2, MIPIa (CCL3), MIPI (CCL5), CCL21, CpG oligodeoxynucleotides, and anti-tumor peptides (e.g., anti-microbial peptides having anti-tumor activity, see, e.g., Gaspar, D. et al. Front Microbiol. 2013; 4: 294; Chu, H. et al. PLoS One. 2015; 10(5): e0126390, and website:aps.unmc.edu/AP/main.php).

Provided herein, in various embodiments, is a multicistronic expression system comprising: (a) an exogenous polynucleotide sequence encoding a first cytokine; (b) an exogenous polynucleotide sequence encoding a second cytokine; and (c) an exogenous polynucleotide sequence encoding an activating chimeric antigen receptor (aCAR), optionally wherein the aCAR comprises: (i) a first antigen-binding domain, (ii) one or more intracellular signaling domains that stimulate an immune response, and (iii) one or more polypeptides selected from the group consisting of a signal peptide, a transmembrane domain, a hinge domain, a spacer region, one or more peptide linkers, and combinations thereof; and (d) an exogenous polynucleotide sequence encoding an inhibitory CAR (iCAR), wherein each exogenous polynucleotide sequence comprises a 5′ end and a 3′ end.

Also provided herein, in various embodiments, is a multicistronic expression system comprising: (a) an exogenous polynucleotide sequence encoding a first cytokine; (b) an exogenous polynucleotide sequence encoding a second cytokine; and (c) an exogenous polynucleotide sequence encoding an activating chimeric antigen receptor (aCAR), wherein each exogenous polynucleotide sequence comprises a 5′ end and a 3′ end, and wherein the aCAR comprises: (i) a first antigen-binding domain that binds to a target selected from: CEA, CEACAM1, CEACAM5, and CEACAM6, optionally wherein the first antigen-binding domain of the aCAR binds CEACAM5, optionally wherein the first antigen binding domain of the aCAR comprises the amino acid sequence set forth in SEQ ID NO: 381; (ii) one or more intracellular signaling domains that stimulate an immune response; and (iii) one or more polypeptides selected from the group consisting of: a signal peptide, a transmembrane domain, a hinge domain, a spacer region, one or more peptide linkers, and combinations thereof.

In some embodiments, (i) the one or more intracellular signaling domains of the aCAR are selected from the group consisting of: CD3-zeta, FcR gamma, FcR beta, CD3 gamma, CD3 delta, CD3 epsilon, CD5, CD22, CD79a, CD79b, CD278, FcεRI, DAP10, DAP12, CD66d, CD97, CD2, ICOS, CD27, CD154, CD8, OX40, 4-1BB, CD28, ZAP40, CD30, GITR, HVEM, DAP10, DAP12, MyD88, 2B4, CD40, PD-1, LFA-1, CD7, LIGHT, NKG2C, B7-H3, an MHC class I molecule, a TNF receptor protein, an Immunoglobulin-like protein, a cytokine receptor, an integrin, a SLAM protein, an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLAl, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and combinations thereof, and/or (ii) the aCAR comprises a hinge domain selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof, and/or (iii) the aCAR comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, and BTLA, and/or (iv) the aCAR comprises a signal peptide selected from the group consisting of: IgE, IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, CXCL12, IL21, CD8, NKG2D, TNFR2, GMCSF, and GM-CSFRa.

In some embodiments, the iCAR comprises: (a) a second antigen-binding domain; (b) one or more intracellular signaling domains that inhibit an immune response; and (c) one or more polypeptides selected from the group consisting of: a signal peptide, a transmembrane domain, a hinge domain, a spacer region, one or more peptide linkers, and combinations thereof. In some embodiments, the second antigen-binding domain of the iCAR binds VSIG2, optionally wherein: (i) the iCAR comprises an LIR1 intracellular inhibitory domain, optionally wherein the intracellular inhibitory domain comprises the amino acid sequence set forth in SEQ ID NO: 387, or (ii) the iCAR comprises an SIRPα intracellular inhibitory domain, optionally wherein the intracellular inhibitory domain comprises the amino acid sequence set forth in SEQ ID NO: 385.

In some embodiments, (i) the iCAR comprises a hinge domain selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof, and/or (ii) the iCAR comprises a transmembrane domain selected from the group consisting of PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, SIRPα, and BTLA, and/or (iii) the iCAR comprises a signal peptide selected from the group consisting of: IgE, IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, CXCL12, IL21, CD8, NKG2D, TNFR2, GMCSF, and GM-CSFRa.

In some embodiments, (i) the exogenous polynucleotide encoding the first cytokine, the exogenous polynucleotide encoding the second cytokine, the exogenous polynucleotide encoding the aCAR, and the exogenous polynucleotide encoding the iCAR are comprised within a single expression vector, or (ii) the exogenous polynucleotide encoding the first cytokine, the exogenous polynucleotide encoding the second cytokine, and the exogenous polynucleotide encoding the aCAR are comprised within a first expression vector, and the exogenous polynucleotide encoding the iCAR is comprised within a second expression vector. In some embodiments, the multicistronic expression system further comprises ribosome skipping sites between each exogenous polynucleotide.

In some embodiments, at least one of the first and the second cytokines is a controlled release cytokine having the formula:

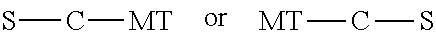

wherein, S comprises a secretable effector molecule; C comprises a protease cleavage site; and MT comprises a cell membrane tethering domain. In some embodiments, (i) the protease cleavage site is cleaved by ADAM10 and/or ADAM17, and/or (ii) the protease cleavage site comprises the amino acid sequence set forth in SEQ ID NO: 180 or SEQ ID NO: 191, and/or (iii) the cell membrane tethering domain comprises a transmembrane domain selected from the group consisting of: B7-1, PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, LIR1, and BTLA, optionally wherein the cell membrane tethering domain comprises a B7-1 transmembrane domain comprising the amino acid sequence set forth in SEQ ID NO: 219.

In some embodiments, (i) the first cytokine is IL15, optionally wherein the IL15 comprises the amino acid sequence set forth in SEQ ID NO: 285, or optionally wherein the IL15 is controlled-release IL15 (crIL15), and/or (ii) the second cytokine is IL21, optionally wherein the IL21 comprises the amino acid sequence set forth in SEQ ID NO: 360, or optionally wherein the IL21 is controlled-release IL21 (crIL21), and/or (iii) the first or second cytokine comprises an amino acid sequence set forth in any one of SEQ ID NOs: 355-359, 361, and 391, and/or (iv) the first or second cytokine is encoded by a nucleic acid sequence set forth in any one of SEQ ID NOs: 367-372, and 392.

Also provided herein, in various embodiments, is a multicistronic expression system comprising: (a) an exogenous polynucleotide sequence encoding a first cytokine; (b) an exogenous polynucleotide sequence encoding a second cytokine; and (c) an exogenous polynucleotide sequence encoding a chimeric antigen receptor (CAR), wherein each exogenous polynucleotide sequence comprises a 5′ end and a 3′ end.

Also provided herein, in various embodiments, is an engineered cell comprising the multicistronic expression system provided herein. In some embodiments, the engineered cell is an immune cell, optionally wherein the engineered cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell, optionally wherein the engineered cell is an NK cell.

Also provided herein, in various embodiments, is a pharmaceutical composition comprising the engineered cell provided herein, and a pharmaceutically acceptable carrier.

Also provided herein, in various embodiments, is a method of treating a disease in a subjected in needed thereof, the method comprising administering a therapeutically effective dose of the engineered cell or the pharmaceutical composition of claim provided herein to the subject, optionally wherein: (i) the disease is a cancer, and/or (ii) the isolated cell is allogenic to the subject or autologous to the subject.

Also provided herein, in various embodiments, is a method of manufacturing an engineered cell, the method comprising transducing an isolated cell with the multicistronic expression system provided herein, optionally wherein: (i) the isolated cell is an immune cell, and/or (ii) the isolated cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell, optionally wherein the isolated cell is an NK cell.

Also provided herein, in various embodiments, is a multicistronic expression system comprising: (a) an exogenous polynucleotide sequence encoding a first cytokine; (b) an exogenous polynucleotide sequence encoding a second cytokine; and (c) an exogenous polynucleotide sequence encoding a chimeric antigen receptor (CAR), wherein each exogenous polynucleotide sequence comprises a 5′ end and a 3′ end. In certain embodiments, In certain embodiments, at least one of the first and the second cytokines is a controlled release cytokine.

In certain embodiments, each controlled release cytokine has the formula:

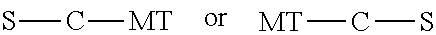

wherein S comprises a secretable effector molecule; C comprises a protease cleavage site; and MT comprises a cell membrane tethering domain. In certain embodiments, the protease cleavage site is cleaved by ADAM10 and/or ADAM17. In certain embodiments, the protease cleavage site comprises the amino acid sequence set forth in SEQ ID NO: 180 or SEQ ID NO: 191. In certain embodiments, the cell membrane tethering domain comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, LIR1, B7-1, and BTLA. In certain embodiments, the cell membrane tethering domain comprises a B7-1 transmembrane domain comprising the amino acid sequence set forth in SEQ ID NO: 219.

In certain embodiments, the first cytokine is IL15. In certain embodiments, the IL15 comprises the amino acid sequence set forth in SEQ ID NO: 285. In certain embodiments, the IL15 is controlled-release IL15 (crIL15). In certain embodiments, the second cytokine is IL21. In certain embodiments, comprises the amino acid sequence set forth in SEQ ID NO: 360. In certain embodiments, the IL21 is controlled-release IL21 (crIL21).

In certain embodiments, the first or second cytokine comprises an amino acid sequence set forth in any one of SEQ ID NOs: 355-359, 361, and 391. In certain embodiments, the first or second cytokine is encoded by a nucleic acid sequence set forth in any one of SEQ ID NOs: -367-372, and 392.

In certain embodiments, the multicistronic expression comprises an exogenous polynucleotide sequence encoding an activating CAR (aCAR) and an exogenous polynucleotide sequence encoding an inhibitory CAR (iCAR). In certain embodiments, the aCAR comprises: (a) a first antigen-binding domain; (b) one or more intracellular signaling domains that stimulate an immune response; and (c) one or more polypeptides selected from the group consisting of: a signal peptide, a transmembrane domain, a hinge domain, a spacer region, one or more peptide linkers, and combinations thereof. In certain embodiments, the first antigen-binding domain of the aCAR binds an antigen selected from: CEA, CEACAM1, CEACAM5, and CEACAM6. In certain embodiments, the first antigen-binding domain of the aCAR binds CEA, CEACAM1, CEACAM5, and CEACAM6. In certain embodiments, the first antigen-binding domain of the aCAR binds CEACAM5. In certain embodiments, the first antigen binding domain of the aCAR comprises the amino acid sequence set forth in SEQ ID NO: 381.

In certain embodiments, the one or more intracellular signaling domains of the aCAR are selected from the group consisting of CD3-zeta, FcR gamma, FcR beta, CD3 gamma, CD3 delta, CD3 epsilon, CD5, CD22, CD79a, CD79b, CD278, FcεRI, DAP10, DAP12, CD66d, CD97, CD2, ICOS, CD27, CD154, CD8, OX40, 4-1BB, CD28, ZAP40, CD30, GITR, HVEM, DAP10, DAP12, MyD88, 2B4, CD40, PD-1, LFA-1, CD7, LIGHT, NKG2C, B7-H3, an MHC class I molecule, a TNF receptor protein, an Immunoglobulin-like protein, a cytokine receptor, an integrin, a SLAM protein, an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLAl, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and combinations thereof. In certain embodiments, the aCAR comprises a hinge domain selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof. In certain embodiments, the aCAR comprises a transmembrane domain selected from the group consisting of PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, and BTLA. In certain embodiments, the aCAR comprises a signal peptide selected from the group consisting of: IgE, IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, CXCL12, IL21, CD8, NKG2D, TNFR2, GMCSF, and GM-CSFRa.

In certain embodiments, the aCAR comprises an amino acid sequence set forth in any one of SEQ ID NOs: 362-365. In certain embodiments, the aCAR is encoded by a nucleic acid sequence set forth in any one of SEQ ID NOs: 373-376.

In certain embodiments, the iCAR comprises: (a) a second antigen-binding domain; (b) one or more intracellular signaling domains that inhibit an immune response; and (c) one or more polypeptides selected from the group consisting of: a signal peptide, a transmembrane domain, a hinge domain, a spacer region, one or more peptide linkers, and combinations thereof. In certain embodiments, the second antigen-binding domain of the iCAR binds VSIG2.

In certain embodiments, the iCAR comprises an LIR1 intracellular inhibitory domain. In certain embodiments, the intracellular inhibitory domain comprises the amino acid sequence set forth in SEQ ID NO: 387. In certain embodiments, the iCAR comprises an SIRPα intracellular inhibitory domain. In certain embodiments, the intracellular inhibitory domain comprises the amino acid sequence set forth in SEQ ID NO: 385.

In certain embodiments, the iCAR comprises a hinge domain selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof. In certain embodiments, the iCAR comprises a transmembrane domain selected from the group consisting of PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, SIRPα, and BTLA. In certain embodiments, the iCAR comprises a signal peptide selected from the group consisting of: IgE, IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, CXCL12, IL21, CD8, NKG2D, TNFR2, GMCSF, and GM-CSFRa.

In certain embodiments, the iCAR comprises the amino acid sequence set forth in SEQ ID NO: 366. In certain embodiments, the iCAR is encoded by the nucleic acid sequence set forth in SEQ ID NO: 377.

In certain embodiments, the exogenous polynucleotide encoding the first cytokine, the exogenous polynucleotide encoding the second cytokine, the exogenous polynucleotide encoding the aCAR, and the exogenous polynucleotide encoding the iCAR are comprised within a single expression vector. In certain embodiments, the exogenous polynucleotide encoding the first cytokine, the exogenous polynucleotide encoding the second cytokine, and the exogenous polynucleotide encoding the aCAR are comprised within a first expression vector, and the exogenous polynucleotide encoding the iCAR is comprised within a second expression vector. In certain embodiments, each exogenous polynucleotide sequence further comprises a promoter sequence at the 5′ end. In certain embodiments, the promoter is a constitutive promoter or an inducible promoter. In certain embodiments, the multicistronic expression system provided herein, further comprises ribosome skipping sites between each exogenous polynucleotide.

Also provided herein, in various embodiments, is an engineered cell comprising the multicistronic expression system provided herein. In certain embodiments, the engineered cell is an immune cell. In certain embodiments, the engineered cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell. In certain embodiments, the engineered cell is an NK cell.

Also provided herein, in various embodiments, is a pharmaceutical composition comprising the engineered cell provided herein and a pharmaceutically acceptable carrier.

Also provided herein, in various embodiments, is a method of treating a disease in a subjected in needed thereof, the method comprising administering a therapeutically effective dose of the engineered cell or the pharmaceutical composition provided herein to the subject. In certain embodiments, the disease is a cancer. In certain embodiments, the isolated cell is allogenic to the subject. In certain embodiments, the isolated cell is autologous to the subject.

Also provided herein, in various embodiments, is a method of manufacturing an engineered cell, the method comprising transducing an isolated cell with the multicistronic expression system provided herein. In certain embodiments, the isolated cell is an immune cell. In certain embodiments, the isolated cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell. In certain embodiments, the isolated cell is an NK cell.

Also provided herein, in various embodiments, is an immunoresponsive cell comprising: (a) an exogenous polynucleotide encoding a first cytokine; (b) an exogenous polynucleotide encoding a second cytokine; and (c) an exogenous polynucleotide encoding a chimeric antigen receptor (CAR).

### BRIEF DESCRIPTION OF THE DRAWINGS

FIGS. 1A-ID illustrate a schematic of a cytokine-CAR bidirectional construct in head-to-head directionality (FIG. 1A), head-to-tail directionality (FIG. 1B), tail-to-tail directionality (FIG. 1C), and an exemplary anti-GPC3 CAR+IL15 bidirectional construct (FIG. 1D).

FIG. 2 provides CAR expression plots assessed by flow cytometry for cells transduced with lentivirus encoding a CAR+IL15 bidirectional construct and cells transduced with a lentivirus encoding the CAR-only (day 7).

FIG. 3 provides CAR expression plots assessed by flow cytometry for cells transduced with retrovirus encoding a CAR+IL15 bidirectional construct and cells transduced with a retrovirus encoding the CAR-only (day 7).

FIG. 4 provides CAR expression plots assessed by flow cytometry for cells transduced with lentivirus encoding a CAR+IL15 bidirectional construct and cells transduced with a lentivirus encoding the CAR-only (day 15).

FIG. 5 provides CAR expression plots assessed by flow cytometry for cells transduced with retrovirus encoding a CAR+IL15 bidirectional construct and cells transduced with a retrovirus encoding the CAR-only (day 15).

FIG. 6 provides IL15 levels assessed by immunoassay for NK cells transduced with lentiviruses encoding CAR+IL15 bidirectional construct (“Lenti”) or γ-retroviruses encoding CAR+IL15 bidirectional constructs (“SinVec”).

FIG. 7 provides killing by NK cells transduced with lentiviruses encoding CAR-only or CAR+IL15 bidirectional constructs, as assessed by a co-culture killing assay.

FIG. 8 provides killing by NK cells transduced with γ-retroviruses encoding CAR-only or CAR+IL15 bidirectional constructs, as assessed by a co-culture killing assay.

FIG. 9 (SEQ ID NO:443) illustrates schematics for bidirectionally orientated constructs, including IL12 expression cassettes having mRNA destabilization elements in the 3′ untranslated region.

FIG. 10 provides IL12 levels assessed by immunoassay for NK cells transduced with bidirectional constructs including an inducible IL12 expression cassette and an expression cassette encoding a synthetic transcription factor.

FIG. 11 illustrates a schematic of bidirectional construct encoding a cleavable release IL15.

FIG. 12 provides a summary of IL15 bicistronic constructs tested and performance in functional assays.

FIG. 13A and FIG. 13B provide expression plots as assessed by flow cytometry for NK cells transduced with SB06251, SB06257, and SB06254, for GPC3 CAR and IL15. Two independent replicates are shown (FIG. 13A and FIG. 13B).

FIG. 14A and FIG. 14B provides secreted IL15 levels as assessed by immunoassay for NK cells transduced with SB06251, SB06257, and SB06254. Two independent replicates are shown (FIG. 14A and FIG. 14B).

FIG. 15A and FIG. 15B provide cell growth of target cell population following co-culture with NK cells transduced with SB06251, SB06257, and SB06254. Two independent replicates are shown (FIG. 15A and FIG. 15B).

FIG. 16 provides target cell counts in a serial-killing assay when co-cultured with NK cells transduced with SB06251, SB06257, and SB06254.

FIG. 17A and FIG. 17B provide expression plots as assessed by flow cytometry for NK cells transduced with SB06252, SB06258, and SB06255, for GPC3 CAR and IL15. Two independent replicates are shown (FIG. 17A and FIG. 17B).

FIG. 18A and FIG. 18B provide secreted IL15 levels as assessed by immunoassay for NK cells tranduced with SB06252, SB06258, and SB06255. Two independent replicates are shown (FIG. 18A and FIG. 18B).

FIG. 19A and FIG. 19B provide cell growth of target cell population following co-culture with NK cells tranduced with SB06252, SB06258, and SB06255. Two independent replicates are shown (FIG. 19A and FIG. 19B).

FIG. 20 provides target cell counts in a serial-killing assay when co-cultured with NK cells transduced with SB06252, SB06258, and SB06255.

FIG. 21A and FIG. 21B provide expression plots as assessed by flow cytometry for NK cells transduced with bicistronic constructs SB06261, SB6294, and SB6298, for GPC3 CAR and IL15. Two independent replicates are shown (FIG. 21A and FIG. 21B).

FIG. 22A and FIG. 22B provide secreted IL15 levels as assessed by immunoassay for NK cells tranduced with SB06261, SB6294, and SB6298. Two independent replicates are shown (FIG. 22A and FIG. 22B).

FIG. 23A and FIG. 23B provide cell growth of target cell population following co-culture with NK cells tranduced with SB06252, SB06258, and SB06255. Two independent replicates are shown (FIG. 23A and FIG. 23B).

FIG. 24A and FIG. 24B provide characterization of cleavable release IL15 bicstronic constructs SB06691, SB06692, and SB06693. Expression plots as assessed by flow cytometry for NK cells transduced with SB06691, SB06692, and SB06693, for GPC3 CAR and IL15, are shown in FIG. 24A. Secreted IL15 levels as assessed by immunoassay for NK cells tranduced with SB06691, SB06692, and SB06693 are shown in FIG. 24B.

FIG. 25 illustrates a schematic of a bidirectional construct encoding a cleavable release IL12.

FIG. 26 provides a dose-response curve of IL12 secretion for NK cells following treatment with grazoprevir (GRZ).

FIG. 27A and FIG. 27B provide in vivo mouse data demonstrating IL12 levels in mouse blood following injection with NK cells tranduced with SB04599, SB05042, and SB05058. IL12 levels are shown in FIG. 27A and IL12 fold change is shown in FIG. 27B.

FIGS. 28A-28C provide characterization of cells transduced with different constructs expressing the GPC3 CAR and IL15. FIG. 28A shows flow cytometry plots demonstrating expression of GPC3 CAR, membrane bound IL15, and respective copy numbers on NK cells transduced with different GPC3 CAR/IL15 expression constructs. FIG. 28B shows measurement of secreted IL15. FIG. 28C shows cell killing of HepG2 as assessed by a serial killing assay.

FIG. 29A and FIG. 29B provide additional data of serial killing using transduced NK Cells. FIG. 29A shows serial killing of HepG2 cells. FIG. 29B shows serial killing of HuH-7 cells.

FIG. 30A and FIG. 30B provide data assessing transduced NK cell function using rapid expansion (G-Rex). FIG. 30A shows expression of GPC3 CAR, membrane bound IL 15(mIL15), and secreted IL15 (sIL15). FIG. 30B shows serial killing of the transduced NK cells.

FIG. 31 provides results from a xenograft tumor model as measured by bioluminescence imaging, in which mice are injected with NK cells.

FIG. 32A and FIG. 32B provide the results of a xenograft tumor model in mice that are injected with NK cells and summary. FIG. 32A provides a survival curve of mice treated with NK cells. FIG. 32B provides a summary of the median survival of mice treated with the NK cells.

FIG. 33 provides results of a BLI experiment to assess tumor reduction in mice injected with NK cells.

FIG. 34 provides a quantification of each condition in terms of BLI measurements that were normalized to day 10.

FIG. 35A and FIG. 35B provide results from a xenograft tumor (HepG2) mouse model in which mice were injected three times with NK cells over the course of the study. FIG. 35A provides results of mice that were imaged using BLI. FIG. 35B provides a time course of fold change of BLI over the course of the study.

FIG. 36A and FIG. 36B provide the fold change BLI in mice injected with transduced NK cells. FIG. 36A provides results corresponding to measurements performed 13 days after tumor implantation. FIG. 36B provides results corresponding to measurements performed 20 days after tumor implantation.

FIG. 37A and FIG. 37B provide results of tumor reduction in a xenograft model. FIG. 37A shows a summary of the BLI Fold change in two different in vivo experiments. FIG. 37B shows a summary of the normalized mean BLI Fold change in two different in vivo experiments, but the treatment groups are separated, and animal are tracked individually.

FIG. 38A and FIG. 38B provide results from a xenograft tumor model in which NK cells are injected intratumorally. FIG. 38A provides measurements of tumor volume. FIG. 38B shows a survival curve.

FIG. 39A and FIG. 39B provide results for expression of IL12 in the presence or absence of grazoprevir. FIG. 39A provides measurements of concentration and fold change 24 hours after induction with grazoprevir. FIG. 39B provides measurements of concentration and fold change 72 hours after induction.

FIG. 40 provides results from a mouse that was injected NK cells expressing regulated IL12 at different concentrations and throughout the experiment.

FIG. 41 provides expression (GPC3 CAR and IL15) results of co-transduction with the IL12 and GPC3 CAR/IL15 constructs into NK cells.

FIG. 42A and FIG. 42B provide results of secreted IL15 and secreted IL12 expression in the presence or absence of grazoprevir. FIG. 42A provides measurements of secreted IL15 concentration. FIG. 42B provides measurements of secreted IL12 expression.

FIG. 43 provides measurements of secreted IL15 and secreted IL12 of NK cells during a serial killing assay.

FIGS. 44A-44D provide results of a serial killing assay for different co-transductions in NK cells for cell killing of Huh-7 and HepG2 cells. FIG. 44A provides the serial killing results for NK cells co-transduced with SB05042+SB06258. FIG. 44B provides the serial killing results for NK cells co-transduced with SB05042+SB06257. FIG. 44C provides the serial killing results for NK cells co-transduced with SB05042+SB06294. FIG. 44D provides a combination of the results in FIGS. 44A-C.

FIGS. 45A-45D provide results from assessment of the clonal selection of NK cells expressing the GPC3 CAR. FIG. 45A provides results on copies per cell. FIG. 45B provides results of GPC3 CAR expression. FIG. 45C provides results for IL15 expression. FIG. 45D provides measurement of secreted IL15.

FIG. 46A and FIG. 46B provide flow cytometry data of GPC3 CAR and IL15 expression on selected clones transduced with SB06258. FIG. 46A provides results of selected clones. FIG. 46B provides results of selected clones further transduced with SB05042 (IL12).

FIGS. 47A-47D provide data on STAT5 phosphorylation in response to controlled-release IL15 (crIL15). FIG. 47A provides results of STAT5 phosphorylation in NK cells expressing CAR and indicated IL15 constructs. FIG. 47B provides results of STAT5 phosphorylation in CD3+ PBMCs incubated with NK cells expressing CAR and indicated IL15 constructs. FIG. 47C provides results of STAT3 and STAT5 phosphorylation in NK cells expressing indicated IL15 constructs. FIG. 47D provides surface association and secretion of IL15 in NK cells transduced with indicated IL15 constructs.

FIGS. 48A and 48B provide results of target cell killing by CAR-NK cells expressing indicated IL15 constructs. FIG. 48A shows abundance of target cells over time during incubation with CAR-NK cells expressing indicated IL15 constructs. FIG. 48B shows abundance of target cells after 120 hours of incubation with CAR-NK cells expressing indicated IL15 constructs.

FIGS. 49A-49C provide results of tumor cell killing by CAR-NK cells expressing one or two cytokines. FIG. 49A shows abundance of tumor cells over time during incubation with CAR-NK cells expressing indicated cytokines. FIG. 49B shows images of tumor cells incubated with CAR-NK cells expressing indicated cytokines. FIG. 49C shows abundance of tumor cells after 120 hrs of incubation with CAR-NK cells expressing indicated cytokines.

FIGS. 50A and 50B show results of analysis of optimal distribution between membrane-bound and soluble cytokines. FIG. 50A shows surface staining of IL15 (vertical axes) and CAR (horizontal axes) in CAR-NK cells expressing the indicated IL15 constructs. FIG. 50B shows expansion (left panel) and viability (right panel) of CAR-NK cells expressing the indicated IL15 constructs.

FIGS. 51A and 51B show results of analysis of IL15 and IL21 constructs in CAR-NK cells. FIG. 51A shows expansion of CAR-NK cells expressing the indicated cytokine constructs. FIG. 51B shows viability of CAR-NK cells expressing the indicated cytokine constructs.

FIGS. 52A and 52B show results of effects of cytokine expression on survival of CAR-NK cells in absence of cytokines in medium. FIG. 52A shows viability of CAR-NK cells expressing indicated cytokine constructs. FIG. 52B shows fold expansion of CAR-NK cells expressing indicated cytokine constructs.

FIGS. 53A-53C show analysis of activation of CAR NK-cells with co-expression of crIL15 and IL21. FIG. 53A shows flow cytometry analysis of CAR-NK cells expressing indicated cytokines activation as measured by IFNγ (vertical axes) and granzyme B (horizonal axes). FIG. 53B shows quantification of IFNγ (left panel) and granzyme B (right panel) staining in NK cells shown in FIG. 53A. FIG. 53C shows images of target cells following incubations with CAR-NK cells expressing indicated cytokines.

FIGS. 54A-54D show analysis of CAR-NK cell killing of target cells. FIG. 54A shows ratio of CAR-NK cell-mediated killing of control cells versus target-expressing cells after one round of killing. Panels show two separate donors. FIG. 54B shows ratio of CAR-NK cell-mediated killing of control cells versus target-expressing cells after multiple rounds of killing. Panels show two separate donors. FIG. 54C shows images of control (red) or target-expressing (green) following incubation with CAR-NK cells expressing indicated constructs. FIG. 54D shows serial killing results of CAR-NK cells killing target cells.

FIG. 55 shows serial killing of target cells by CAR-NK cells expressing indicated constructs under suppression by culture in the presence of TGFβ.

FIG. 56 shows serial killing of cells expressing inhibitory CAR (iCAR) target antigen by NK cells expressing an iCAR and an activating CAR (aCAR).

FIGS. 57A-57E show in vivo tumor suppression by CAR-NK cells co-expressing crIL15 and IL21. FIG. 57A shows images of tumors at indicated time points in mice treated as indicated. FIG. 57B shows tumor growth over time in mice treated as indicated. FIG. 57C shows progression-free survival over time with mice treated with indicated CAR-NK cells. FIG. 57D shows percent survival over time with mice treated with indicated CAR-NK cells. FIG. 57E shows images of tumors in mice treated as indicated 15 days after tumor engraftment (top panel) and graphs showing percentages of mice with observed tumor reduction compared to untreated control (bottom panel).

FIGS. 58A-58D show persistence of CAR-NK cells expressing crIL15 and IL21 in tumor-bearing mice. FIG. 58A shows percentage of CD45-expressing cells as a percentage of total in intraperitoneal fluid (left panel) and blood (right panel) 27 days following administration. FIG. 58B shows staining of human CD45 (vertical axes) and murine CD45 (horizontal axes) in NK cells expressing indicated constructs 27 days following administration. FIG. 58C shows percentage of CD45-expressing cells as a percentage of total in intraperitoneal fluid 70 days following administration. FIG. 58D shows staining of human CD45 (vertical axes) and murine CD45 (horizontal axes) in NK cells expressing indicated constructs 70 days following administration.

FIGS. 59A and 59B detail screening of various IL15 constructs and combinations with IL7 or IL21. FIG. 59A shows serial killing of target cells at indicated effector to target ratios (E:T) incubated with NK-cells expressing indicated constructs. FIG. 59B shows percentage of NK cells expressing the CAR.

FIGS. 60A-60E detail analysis of IL15 with IL21 or IL7. FIG. 60A shows serial killing of target cells by NK cells expressing control or indicated cytokine constructs. FIG. 60B shows serial killing of target cells by NK cells control or indicated cytokine constructs. FIG. 60C shows serial killing of target cells by NK cells expressing control or indicated IL15 constructs. FIG. 60D shows serial killing of target cells by NK cells expressing control or indicated cytokine constructs. FIG. 60E shows serial killing of target cells by NK cells expressing control or indicated IL15 constructs.

FIGS. 61A-61C detail construction of recombinant IL15 sushi domain-containing proteins. FIG. 61A details the design of the synthetic protein constructs. FIG. 61B shows killing of target cells incubated with control or NK cells expressing indicated constructs. FIG. 61C shows second round killing of target cells incubated with control or NK cells expressing indicated constructs.

FIGS. 62A-62D detail the production of NK cells engineered to express an inhibitory CAR (iCAR) and a controlled-release IL15 (crIL15 or mIL15). FIG. 62A details the percentage of engineered cells expressing iCAR or crIL15 as measured by flow cytometry. FIG. 62B depicts expression of the iCAR or crIL15 in engineered cells as measured by flow cytometry. FIG. 62C depicts the secretion of IL-15 by NK cells engineered to express the indicated constructs. FIG. 62D depicts the secretion of IL-21 by NK cells engineered to express the indicated constructs.

### DETAILED DESCRIPTION

Provided herein, in various embodiments, are multicistronic expression systems. In some embodiments, the multicistronic expression system comprises: (a) an exogenous polynucleotide encoding a first cytokine; (b) an exogenous polynucleotide encoding a second cytokine; and (c) an exogenous polynucleotide encoding a chimeric antigen receptor (CAR). In certain embodiments, the multicistronic expression system comprises an activating CAR (aCAR) and an inhibitory CAR (iCAR).

Also provided herein, in various embodiments, are immunoresponsive cells engineered to have the following:
(a) an exogenous polynucleotide encoding a first cytokine; (b) an exogenous polynucleotide encoding a second cytokine; and (c) an exogenous polynucleotide encoding a chimeric antigen receptor (CAR).

The multicistronic expression system or immunoresponsive cells disclosed herein can include an activation-control polypeptide. The ACP can include a synthetic transcription factor. A synthetic transcription factor is a non-naturally occurring protein that includes a DNA-binding domain and a transcriptional effector domain and is capable of modulating (i.e., activating or repressing) transcription through binding to a cognate promoter recognized by the DNA-binding domain (an ACP-responsive promoter). In some embodiments, the ACP is a transcriptional repressor. In some embodiments, the ACP is a transcriptional activator.

The membrane-cleavable chimeric protein can be engineered such that secretion of the effector molecule can be regulated in a protease-dependent manner. Specifically, the membrane-cleavable chimeric protein can be engineered such that secretion of the effector molecule can be regulated as part of a “Membrane-Cleavable” system, where incorporation of a protease cleavage site (“C”) and a cell membrane tethering domain (“MT”) allow for regulated secretion of an effector molecule in a protease-dependent manner. Without wishing to be bound by theory, the components of the Membrane-Cleavable system present in the membrane-cleavable chimeric protein generally regulate secretion through the below cellular processes:
MT: The cell membrane tethering domain contains a transmembrane domain (or a transmembrane-intracellular domain) that directs cellular-trafficking of the chimeric protein such that the protein is inserted into, or otherwise associated with, a cell membrane (“tethered”)
C: Following expression and localization of the chimeric protein into the cell membrane, the protease cleavage site directs cleavage of the chimeric protein such that the effector molecule is released (“secreted”) into the extracellular space. Generally, the protease cleavage site is protease-specific, including sites engineered to be protease-specific. The protease cleavage site can be selected or engineered to achieve optimal protein expression, cell-type specific cleavage, cell-state specific cleavage, and/or cleavage and release of the payload at desired kinetics (e.g., ratio of membrane-bound to secreted chimeric protein levels)

In some aspects, membrane-cleavable chimeric proteins (or engineered nucleic acids encoding the membrane-cleavable chimeric proteins) are provided for herein having a protein of interest (e.g., any of the effector molecules described herein), a protease cleavage site, and a cell membrane tethering domain.

An “effector molecule,” refers to a molecule (e.g., a nucleic acid such as DNA or RNA, or a protein (polypeptide) or peptide) that binds to another molecule and modulates the biological activity of that molecule to which it binds. For example, an effector molecule may act as a ligand to increase or decrease enzymatic activity, gene expression, or cell signaling. Thus, in some embodiments, an effector molecule modulates (activates or inhibits) different immunomodulatory mechanisms. By directly binding to and modulating a molecule, an effector molecule may also indirectly modulate a second, downstream molecule.

In general, for all membrane-cleavable chimeric proteins described herein, an effector molecule is a cytokine or active fragment thereof (the secretable effector molecule referred to as “S” in the formula S-C-MT or MT-C-S) that includes a cytokine or active fragments thereof.

The term “modulate” encompasses maintenance of a biological activity, inhibition (partial or complete) of a biological activity, and stimulation/activation (partial or complete) of a biological activity. The term also encompasses decreasing or increasing (e.g., enhancing) a biological activity. Two different effector molecules are considered to “modulate different tumor-mediated immunosuppressive mechanisms” when one effector molecule modulates a tumor-mediated immunosuppressive mechanism (e.g., stimulates T cell signaling) that is different from the tumor-mediated immunosuppressive mechanism modulated by the other effector molecule (e.g., stimulates antigen presentation and/or processing).

Modulation by an effector molecule may be direct or indirect. Direct modulation occurs when an effector molecule binds to another molecule and modulates activity of that molecule. Indirect modulation occurs when an effector molecule binds to another molecule, modulates activity of that molecule, and as a result of that modulation, the activity of yet another molecule (to which the effector molecule is not bound) is modulated.

In some embodiments, modulation of a tumor-mediated immunosuppressive mechanism by at least one effector molecule results in an increase in an immunostimulatory and/or anti-tumor immune response (e.g., systemically or in the tumor microenvironment) by at least 10% (e.g., 10%, 20%, 30%, 40%, 50%, 60%, 70%, 80%, 90%, 100%, or 200%). For example, modulation of a tumor-mediated immunosuppressive mechanism may result in an increase in an immunostimulatory and/or anti-tumor immune response by at least 20%, at least 30%, at least 40%, at least 50%, at least 60%, at least 70%, at least 80%, at least 90%, at least 100%. In some embodiments, modulation of a tumor-mediated immunosuppressive mechanism results in an increase in an immunostimulatory and/or anti-tumor immune response 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%. It should be understood that “an increase” in an immunostimulatory and/or anti-tumor immune response, for example, systemically or in a tumor microenvironment, is relative to the immunostimulatory and/or anti-tumor immune response that would otherwise occur, in the absence of the effector molecule(s).

In some embodiments, modulation of a tumor-mediated immunosuppressive mechanism by at least one effector molecule results in an increase in an immunostimulatory and/or anti-tumor immune response (e.g., systemically or in the tumor microenvironment) by at least 2 fold (e.g., 2, 3, 4, 5, 10, 25, 20, 25, 50, or 100 fold). For example, modulation of a tumor-mediated immunosuppressive mechanism may result in an increase in an immunostimulatory and/or anti-tumor immune response by at least 3 fold, at least 5 fold, at least 10 fold, at least 20 fold, at least 50 fold, or at least 100 fold. In some embodiments, modulation of a tumor-mediated immunosuppressive mechanism results in an increase in an immunostimulatory and/or anti-tumor immune response by 2-10, 2-20, 2-30, 2-40, 2-50, 2-60, 2-70, 2-80, 2-90, or 2-100 fold.

Non-limiting examples of immunostimulatory and/or anti-tumor immune mechanisms include T cell signaling, activity and/or recruitment, antigen presentation and/or processing, natural killer cell-mediated cytotoxic signaling, activity and/or recruitment, dendritic cell differentiation and/or maturation, immune cell recruitment, pro-inflammatory macrophage signaling, activity and/or recruitment, stroma degradation, immunostimulatory metabolite production, stimulator of interferon genes (STING) signaling (which increases the secretion of IFN and Th1 polarization, promoting an anti-tumor immune response), and/or Type I interferon signaling. An effector molecule may stimulate at least one (one or more) of the foregoing immunostimulatory mechanisms, thus resulting in an increase in an immunostimulatory response. Changes in the foregoing immunostimulatory and/or anti-tumor immune mechanisms may be assessed, for example, using in vitro assays for T cell proliferation or cytotoxicity, in vitro antigen presentation assays, expression assays (e.g., of particular markers), and/or cell secretion assays (e.g., of cytokines).

In some embodiments, modulation of a tumor-mediated immunosuppressive mechanism by at least one effector molecule results in a decrease in an immunosuppressive response (e.g., systemically or in the tumor microenvironment) by at least 10% (e.g., 10%, 20%, 30%, 40%, 50%, 60%, 70%, 80%, 90%, 100%, or 200%). For example, modulation of a tumor-mediated immunosuppressive mechanism may result in a decrease in an immunosuppressive response by at least 20%, at least 30%, at least 40%, at least 50%, at least 60%, at least 70%, at least 80%, at least 90%, at least 100%. In some embodiments, modulation of a tumor-mediated immunosuppressive mechanism results in a decrease in an immunosuppressive response 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%. It should be understood that “a decrease” in an immunosuppressive response, for example, systemically or in a tumor microenvironment, is relative to the immunosuppressive response that would otherwise occur, in the absence of the effector molecule(s).

In some embodiments, modulation of a tumor-mediated immunosuppressive mechanism by at least one effector molecule results in a decrease in an immunosuppressive response (e.g., systemically or in the tumor microenvironment) by at least 2 fold (e.g., 2, 3, 4, 5, 10, 25, 20, 25, 50, or 100 fold). For example, modulation of a tumor-mediated immunosuppressive mechanism may result in a decrease in an immunosuppressive response by at least 3 fold, at least 5 fold, at least 10 fold, at least 20 fold, at least 50 fold, or at least 100 fold. In some embodiments, modulation of a tumor-mediated immunosuppressive mechanism results in a decrease in an immunosuppressive response by 2-10, 2-20, 2-30, 2-40, 2-50, 2-60, 2-70, 2-80, 2-90, or 2-100 fold.

Non-limiting examples of immunosuppressive mechanisms include negative costimulatory signaling, pro-apoptotic signaling of cytotoxic cells (e.g., T cells and/or NK cells), T regulatory (Treg) cell signaling, tumor checkpoint molecule production/maintenance, myeloid-derived suppressor cell signaling, activity and/or recruitment, immunosuppressive factor/metabolite production, and/or vascular endothelial growth factor signaling. An effector molecule may inhibit at least one (one or more) of the foregoing immunosuppressive mechanisms, thus resulting in a decrease in an immunosuppressive response. Changes in the foregoing immunosuppressive mechanisms may be assessed, for example, by assaying for an increase in T cell proliferation and/or an increase in IFNγ production (negative co-stimulatory signaling, T_{reg} cell signaling and/or MDSC); Annexin V/PI flow staining (pro-apoptotic signaling); flow staining for expression, e.g., PDL1 expression (tumor checkpoint molecule production/maintenance); ELISA, LUMINEX®, RNA via qPCR, enzymatic assays, e.g., IDO tryptophan catabolism (immunosuppressive factor/metabolite production); and phosphorylation of PI3K, Akt, p38 (VEGF signaling).

In some embodiments, effector molecules function additively: the effect of two effector molecules, for example, may be equal to the sum of the effect of the two effector molecules functioning separately. In other embodiments, effector molecules function synergistically: the effect of two effector molecules, for example, may be greater than the combined function of the two effector molecules.

Effector molecules that modulate tumor-mediated immunosuppressive mechanisms and/or modify tumor microenvironments may be any of the cytokines described herein.

In some embodiments, at least one of the effector molecules stimulates an immunostimulatory mechanism in the tumor microenvironment and/or inhibits an immunosuppressive mechanism in the tumor microenvironment.

In some embodiments, at least one of the effector molecules (a) stimulates T cell signaling, activity and/or recruitment, (b) stimulates antigen presentation and/or processing, (c) stimulates natural killer cell-mediated cytotoxic signaling, activity and/or recruitment, (d) stimulates dendritic cell differentiation and/or maturation, (e) stimulates immune cell recruitment, (f) stimulates pro-inflammatory macrophage signaling, activity and/or recruitment or inhibits anti-inflammatory macrophage signaling, activity and/or recruitment, (g) stimulates stroma degradation, (h) stimulates immunostimulatory metabolite production, (i) stimulates Type I interferon signaling, 0j) inhibits negative costimulatory signaling, (k) inhibits pro-apoptotic signaling of anti-tumor immune cells, (1) inhibits T regulatory (T_{reg}) cell signaling, activity and/or recruitment, (in) inhibits tumor checkpoint molecules, (n) stimulates stimulator of interferon genes (STING) signaling, (o) inhibits myeloid-derived suppressor cell signaling, activity and/or recruitment, (p) degrades immunosuppressive factors/metabolites, (q) inhibits vascular endothelial growth factor signaling, and/or (r) directly kills tumor cells.

Non-limiting examples of cytokines are listed in Table 1 and specific sequences encoding exemplary effector molecules are listed in Table 2. Effector molecules can be human, such as those listed in Table 1 or Table 2 or human equivalents of murine effector molecules listed in Table 1 or Table 2. Effector molecules can be human-derived, such as the endogenous human effector molecule or an effector molecule modified and/or optimized for function, e.g., codon optimized to improve expression, modified to improve stability, or modified at its signal sequence (see below). Various programs and algorithms for optimizing function are known to those skilled in the art and can be selected based on the improvement desired, such as codon optimization for a specific species (e.g., human, mouse, bacteria, etc.).

TABLE 1
Exemplary Effector Molecules

| Effector name | Category | Function |
|---|---|---|
| IFNbeta | Cytokine | T cell response, tumor cell killing |
| IFNgamma | Cytokine | T cell response, tumor cell killing |
| IL12 (e.g., IL12p70 | Cytokine | T cells, NK cells |
| fusion) | | |
| IL1-beta | Cytokine | T cells, NK cells |
| IL15 | Cytokine | Stimulates T-cells and NK |
| IL2 | Cytokine | Stimulates T-cells and NK |
| IL21 | Cytokine | Stimulates T-cells |
| IL24 | Cytokine | Stimulates T-cells |
| IL36-gamma | Cytokine | Stimulates T-cells |
| IL7 | Cytokine | Stimulates T-cells |
| IL22 | Cytokine | Stimulates T-cells |
| IL18 | Cytokine | Stimulates T-cells |

TABLE 2
Sequences encoding exemplary effector molecules

IL12 (Human) (SEQ ID NO: 56)
ATGTGCCATCAGCAGCTTGTCATATCTTGGTTTTCACTTGTATTCCTGGCCAGCCCTTTG
GTTGCGATCTGGGAGCTCAAGAAGGATGTGTACGTTGTAGAGCTGGACTGGTACCCCGAT
GCTCCCGGTGAGATGGTCGTTTTGACATGTGACACTCCAGAAGAGGACGGTATTACGTGG
ACTCTGGACCAGTCCTCCGAAGTTCTTGGTTCTGGTAAGACTCTGACTATCCAGGTGAAA
GAATTTGGGGATGCGGGACAATACACATGCCACAAGGGAGGCGAGGTGTTGTCTCATAGT
TTGCTGCTTCTCCACAAGAAAGAGGATGGAATCTGGAGCACCGACATACTCAAGGATCAA
AAGGAACCCAAAAATAAGACATTTCTGCGATGTGAGGCTAAGAACTATAGTGGCCGCTTC
ACTTGTTGGTGGCTGACTACCATCAGCACAGATCTCACGTTTTCAGTAAAAAGTAGTAGA
GGTTCAAGTGATCCTCAAGGGGTAACGTGCGGTGCTGCAACACTGTCTGCTGAACGCGTA
AGAGGAGATAATAAGGAGTACGAGTATTCCGTAGAATGCCAAGAGGACAGTGCTTGTCCT
GCGGCCGAGGAGTCTCTCCCAATAGAAGTGATGGTGGACGCGGTGCATAAACTGAAATAT
GAGAACTACACAAGCAGTTTTTTTATAAGAGATATCATCAAGCCCGATCCGCCGAAGAAT
TTGCAACTTAAACCGCTTAAAAACTCACGCCAGGTTGAAGTATCCTGGGAGTATCCGGAT
ACATGGTCAACACCACACAGCTATTTTTCCCTTACCTTCTGTGTGCAGGTCCAAGGGAAG
AGCAAAAGGGAGAAGAAGGACAGGGTATTCACTGATAAAACTTCCGCGACGGTCATCTGC
CGAAAAAACGCTAGTATATCTGTACGGGCGCAGGATAGGTACTATAGTTCTTCTTGGTCT
GAGTGGGCCTCAGTTCCGTGCTCTGGGGGAGGAAGTGGAGGAGGGTCCGGCGGTGGAAGC
GGGGGAGGGAGTCGCAACTTGCCAGTGGCTACACCAGATCCAGGCATGTTTCCATGTCTG
CATCATTCCCAGAATCTCCTGAGAGCGGTGTCAAATATGCTCCAAAAAGCGAGACAAACA
CTGGAATTTTACCCGTGTACCAGTGAGGAGATTGATCACGAGGACATAACCAAGGACAAG
ACCTCAACTGTAGAAGCGTGTTTGCCGCTGGAGTTGACTAAGAATGAGTCCTGCCTCAAT
TCCAGAGAAACTTCATTCATTACTAACGGCAGTTGTCTTGCATCCCGGAAAACGTCCTTT
ATGATGGCCCTTTGCCTTAGTTCAATTTACGAGGATCTTAAAATGTATCAAGTGGAGTTT
AAAACCATGAATGCTAAACTTCTTATGGACCCCAAACGACAAATTTTTCTGGATCAGAAT
ATGCTTGCCGTGATAGACGAACTCATGCAGGCGCTTAATTTTAACTCCGAAACAGTTCCA
CAAAAATCTAGCCTTGAAGAACCTGATTTTTATAAAACGAAGATTAAACTGTGTATCCTG
CTGCATGCCTTTCGCATCCGAGCTGTCACAATCGATAGGGTTATGTCCTACCTTAACGCG
AGCtaG
IL12p70 (Human; codon optimized; bold denotes signal sequence)
(SEQ ID NO: 57)
ATGTGCCATCAGCAACTCGTCATCTCCTGGTTCTCCCTTGTGTTCCTCGCTTCCCCTCTG
GTCGCCATTTGGGAACTGAAGAAGGACGTCTACGTGGTCGAGCTGGATTGGTACCCGGAC
GCCCCTGGAGAAATGGTCGTGCTGACTTGCGATACGCCAGAAGAGGACGGCATAACCTGG
ACCCTGGATCAGAGCTCCGAGGTGCTCGGAAGCGGAAAGACCCTGACCATTCAAGTCAAG
GAGTTCGGCGACGCGGGCCAGTACACTTGCCACAAGGGTGGCGAAGTGCTGTCCCACTCC
CTGCTGCTGCTGCACAAGAAAGAGGATGGAATCTGGTCCACTGACATCCTCAAGGACCAA
AAAGAACCGAAGAACAAGACCTTCCTCCGCTGCGAAGCCAAGAACTACAGCGGTCGGTTC
ACCTGTTGGTGGCTGACGACAATCTCCACCGACCTGACTTTCTCCGTGAAGTCGTCACGG
GGATCAAGCGATCCTCAGGGCGTGACCTGTGGAGCCGCCACTCTGTCCGCCGAGAGAGTC
AGGGGAGACAACAAGGAATATGAGTACTCCGTGGAATGCCAGGAGGACAGCGCCTGCCCT
GCCGCGGAAGAGTCCCTGCCTATCGAGGTCATGGTCGATGCCGTGCATAAGCTGAAATAC
GAGAACTACACTTCCTCCTTCTTTATCCGCGACATCATCAAGCCTGACCCCCCCAAGAAC
TTGCAGCTGAAGCCACTCAAGAACTCCCGCCAAGTGGAAGTGTCTTGGGAATATCCAGAC
ACTTGGAGCACCCCGCACTCATACTTCTCGCTCACTTTCTGTGTGCAAGTGCAGGGAAAG
TCCAAACGGGAGAAGAAAGACCGGGTGTTCACCGACAAAACCTCCGCCACTGTGATTTGT
CGGAAGAACGCGTCAATCAGCGTCCGGGCGCAGGATAGATACTACTCGTCCTCCTGGAGC
GAATGGGCCAGCGTGCCTTGTTCCGGTGGCGGATCAGGCGGAGGTTCAGGAGGAGGCTCC
GGAGGAGGTTCCCGGAACCTCCCTGTGGCAACCCCCGACCCTGGAATGTTCCCGTGCCTA
CACCACTCCCAAAACCTCCTGAGGGCTGTGTCGAACATGTTGCAGAAGGCCCGCCAGACC
CTTGAGTTCTACCCCTGCACCTCGGAAGAAATTGATCACGAGGACATCACCAAGGACAAG
ACCTCGACCGTGGAAGCCTGCCTGCCGCTGGAACTGACCAAGAACGAATCGTGTCTGAAC
TCCCGCGAGACAAGCTTTATCACTAACGGCAGCTGCCTGGCGTCGAGAAAGACCTCATTC
ATGATGGCGCTCTGTCTTTCCTCGATCTACGAAGATCTGAAGATGTATCAGGTCGAGTTC
AAGACCATGAACGCCAAGCTGCTCATGGACCCGAAGCGGCAGATCTTCCTGGACCAGAAT
ATGCTCGCCGTGATTGATGAACTGATGCAGGCCCTGAATTTCAACTCCGAGACTGTGCCT
CAAAAGTCCAGCCTGGAAGAACCGGACTTCTACAAGACCAAGATCAAGCTGTGCATCCTG
TTGCACGCTTTCCGCATTCGAGCCGTGACCATTGACCGCGTGATGTCCTACCTGAACGCC
AGT
IL12 (Mouse) (SEQ ID NO: 58)
ATGTGTCCACAGAAGCTGACAATAAGTTGGTTTGCCATTGTCCTCCTGGTGAGCCCACTC
ATGGCAATGTGGGAACTCGAAAAGGATGTCTACGTGGTAGAAGTAGATTGGACTCCAGAC
GCGCCAGGGGAGACAGTGAATTTGACATGTGACACACCAGAAGAAGATGACATTACATGG
ACATCTGACCAACGCCATGGCGTAATAGGGAGTGGGAAAACACTCACGATCACAGTTAAA
GAGTTCTTGGATGCTGGTCAATATACTTGCCATAAAGGCGGCGAGACACTCAGCCACTCA
CATTTGCTTTTGCATAAAAAAGAGAATGGCATTTGGAGCACTGAAATACTTAAGAACTTT
AAGAACAAGACATTTCTCAAGTGTGAGGCCCCTAATTACAGCGGCAGGTTCACGTGCTCA
TGGCTGGTCCAGCGCAACATGGACCTCAAGTTTAACATAAAATCTTCTTCCTCTTCACCT
GACTCCAGAGCTGTTACTTGCGGCATGGCTTCTCTGAGCGCAGAAAAAGTAACGTTGGAT
CAAAGAGACTACGAAAAGTACTCTGTTTCTTGTCAAGAGGATGTTACGTGCCCGACGGCC
GAAGAAACGCTTCCAATTGAACTCGCGTTGGAAGCTCGCCAACAAAACAAGTATGAAAAC
TACAGTACAAGCTTCTTTATACGGGATATAATTAAACCCGATCCCCCCAAGAACTTGCAA
ATGAAACCACTTAAGAACAGCCAGGTGGAAGTTTCCTGGGAGTATCCAGACTCATGGAGT
ACTCCTCACAGCTATTTTTCTCTGAAATTCTTTGTAAGGATACAACGGAAGAAAGAGAAG
ATGAAAGAGACCGAGGAGGGTTGTAATCAGAAGGGAGCGTTTCTCGTGGAGAAAACGTCT
ACCGAAGTCCAATGTAAAGGTGGCAATGTGTGCGTCCAAGCTCAGGATAGATACTATAAT
TCAAGTTGCTCCAAGTGGGCCTGTGTTCCATGCCGCGTTCGGAGCGGGGGAGGTAGCGGA
GGAGGTAGTGGGGGTGGGTCAGGAGGAGGGAGTCGAGTTATCCCGGTGTCAGGCCCCGCA
CGCTGCTTGAGCCAGAGTCGCAACCTCCTTAAGACAACAGATGACATGGTGAAAACAGCA
CGCGAAAAGCTTAAACACTACTCTTGTACGGCGGAGGATATTGATCACGAGGATATTACC
CGAGACCAAACTAGCACTTTGAAAACCTGTCTGCCCCTTGAACTTCATAAAAATGAGAGC
TGTCTGGCTACACGAGAGACGTCAAGTACGACTAGGGGCAGCTGTCTCCCGCCGCAAAAG
ACAAGCCTCATGATGACGCTCTGTTTGGGTTCCATTTACGAGGACTTGAAAATGTATCAA
ACGGAGTTCCAGGCTATAAATGCGGCGTTGCAGAACCATAACCATCAACAAATTATACTT
GATAAAGGCATGTTGGTGGCGATTGATGAACTCATGCAGAGTCTCAATCACAACGGGGAA
ACGTTGAGACAGAAACCCCCAGTCGGTGAAGCGGACCCATATCGAGTAAAAATGAAGCTC
TGCATTCTGCTTCACGCATTCAGCACTAGAGTTGTTACCATCAACCGGGTAATGGGATAT
CTCTCCAGTGCGtaG
IL21 (Human; codon optimized; bold denotes signal sequence)
(SEQ ID NO: 59)
ATGGAACGCATTGTGATCTGCCTGATGGTCATCTTCCTGGGCACCTTAGTGCACAAGTCG
AGCAGCCAGGGACAGGACAGGCACATGATTAGAATGCGCCAGCTCATCGATATCGTGGAC
CAGTTGAAGAACTACGTGAACGACCTGGTGCCCGAGTTCCTGCCGGCCCCCGAAGATGTG
GAAACCAATTGCGAATGGTCGGCATTTTCCTGCTTTCAAAAGGCACAGCTCAAGTCCGCT
AACACCGGGAACAACGAACGGATCATCAACGTGTCCATCAAAAAGCTGAAGCGGAAGCCT
CCCTCCACCAACGCCGGACGGAGGCAGAAGCATAGGCTGACTTGCCCGTCATGCGACTCC
TACGAGAAGAAGCCGCCGAAGGAGTTCCTGGAGCGGTTCAAGTCGCTCCTGCAAAAGATG
ATTCATCAGCACCTGTCCTCCCGGACTCATGGGTCTGAGGATTCA
IL12p70_T2A_IL21 (Human; codon optimized;
bold denotes signal sequences) (SEQ ID NO: 60)
ATGTGCCATCAGCAACTCGTCATCTCCTGGTTCTCCCTTGTGTTCCTCGCTTCCCCTCTG
GTCGCCATTTGGGAACTGAAGAAGGACGTCTACGTGGTCGAGCTGGATTGGTACCCGGAC
GCCCCTGGAGAAATGGTCGTGCTGACTTGCGATACGCCAGAAGAGGACGGCATAACCTGG
ACCCTGGATCAGAGCTCCGAGGTGCTCGGAAGCGGAAAGACCCTGACCATTCAAGTCAAG
GAGTTCGGCGACGCGGGCCAGTACACTTGCCACAAGGGTGGCGAAGTGCTGTCCCACTCC
CTGCTGCTGCTGCACAAGAAAGAGGATGGAATCTGGTCCACTGACATCCTCAAGGACCAA
AAAGAACCGAAGAACAAGACCTTCCTCCGCTGCGAAGCCAAGAACTACAGCGGTCGGTTC
ACCTGTTGGTGGCTGACGACAATCTCCACCGACCTGACTTTCTCCGTGAAGTCGTCACGG
GGATCAAGCGATCCTCAGGGCGTGACCTGTGGAGCCGCCACTCTGTCCGCCGAGAGAGTC
AGGGGAGACAACAAGGAATATGAGTACTCCGTGGAATGCCAGGAGGACAGCGCCTGCCCT
GCCGCGGAAGAGTCCCTGCCTATCGAGGTCATGGTCGATGCCGTGCATAAGCTGAAATAC
GAGAACTACACTTCCTCCTTCTTTATCCGCGACATCATCAAGCCTGACCCCCCCAAGAAC
TTGCAGCTGAAGCCACTCAAGAACTCCCGCCAAGTGGAAGTGTCTTGGGAATATCCAGAC
ACTTGGAGCACCCCGCACTCATACTTCTCGCTCACTTTCTGTGTGCAAGTGCAGGGAAAG
TCCAAACGGGAGAAGAAAGACCGGGTGTTCACCGACAAAACCTCCGCCACTGTGATTTGT
CGGAAGAACGCGTCAATCAGCGTCCGGGCGCAGGATAGATACTACTCGTCCTCCTGGAGC
GAATGGGCCAGCGTGCCTTGTTCCGGTGGCGGATCAGGCGGAGGTTCAGGAGGAGGCTCC
GGAGGAGGTTCCCGGAACCTCCCTGTGGCAACCCCCGACCCTGGAATGTTCCCGTGCCTA
CACCACTCCCAAAACCTCCTGAGGGCTGTGTCGAACATGTTGCAGAAGGCCCGCCAGACC
CTTGAGTTCTACCCCTGCACCTCGGAAGAAATTGATCACGAGGACATCACCAAGGACAAG
ACCTCGACCGTGGAAGCCTGCCTGCCGCTGGAACTGACCAAGAACGAATCGTGTCTGAAC
TCCCGCGAGACAAGCTTTATCACTAACGGCAGCTGCCTGGCGTCGAGAAAGACCTCATTC
ATGATGGCGCTCTGTCTTTCCTCGATCTACGAAGATCTGAAGATGTATCAGGTCGAGTTC
AAGACCATGAACGCCAAGCTGCTCATGGACCCGAAGCGGCAGATCTTCCTGGACCAGAAT
ATGCTCGCCGTGATTGATGAACTGATGCAGGCCCTGAATTTCAACTCCGAGACTGTGCCT
CAAAAGTCCAGCCTGGAAGAACCGGACTTCTACAAGACCAAGATCAAGCTGTGCATCCTG
TTGCACGCTTTCCGCATTCGAGCCGTGACCATTGACCGCGTGATGTCCTACCTGAACGCC
AGTAGACGGAAACGCGGAAGCGGAGAGGGCAGAGGCTCGCTGCTTACATGCGGGGACGTG
GAAGAGAACCCCGGTCCGATGGAACGCATTGTGATCTGCCTGATGGTCATCTTCCTGGGC
ACCTTAGTGCACAAGTCGAGCAGCCAGGGACAGGACAGGCACATGATTAGAATGCGCCAG
CTCATCGATATCGTGGACCAGTTGAAGAACTACGTGAACGACCTGGTGCCCGAGTTCCTG
CCGGCCCCCGAAGATGTGGAAACCAATTGCGAATGGTCGGCATTTTCCTGCTTTCAAAAG
GCACAGCTCAAGTCCGCTAACACCGGGAACAACGAACGGATCATCAACGTGTCCATCAAA
AAGCTGAAGCGGAAGCCTCCCTCCACCAACGCCGGACGGAGGCAGAAGCATAGGCTGACT
TGCCCGTCATGCGACTCCTACGAGAAGAAGCCGCCGAAGGAGTTCCTGGAGCGGTTCAAG
TCGCTCCTGCAAAAGATGATTCATCAGCACCTGTCCTCCCGGACTCATGGGTCTGAGGAT
TCA
IL12_2A_CCL21a (Human) (SEQ ID NO: 61)
ATGTGCCATCAGCAGCTTGTCATATCTTGGTTTTCACTTGTATTCCTGGCCAGCCCTTTG
GTTGCGATCTGGGAGCTCAAGAAGGATGTGTACGTTGTAGAGCTGGACTGGTACCCCGAT
GCTCCCGGTGAGATGGTCGTTTTGACATGTGACACTCCAGAAGAGGACGGTATTACGTGG
ACTCTGGACCAGTCCTCCGAAGTTCTTGGTTCTGGTAAGACTCTGACTATCCAGGTGAAA
GAATTTGGGGATGCGGGACAATACACATGCCACAAGGGAGGCGAGGTGTTGTCTCATAGT
TTGCTGCTTCTCCACAAGAAAGAGGATGGAATCTGGAGCACCGACATACTCAAGGATCAA
AAGGAACCCAAAAATAAGACATTTCTGCGATGTGAGGCTAAGAACTATAGTGGCCGCTTC
ACTTGTTGGTGGCTGACTACCATCAGCACAGATCTCACGTTTTCAGTAAAAAGTAGTAGA
GGTTCAAGTGATCCTCAAGGGGTAACGTGCGGTGCTGCAACACTGTCTGCTGAACGCGTA
AGAGGAGATAATAAGGAGTACGAGTATTCCGTAGAATGCCAAGAGGACAGTGCTTGTCCT
GCGGCCGAGGAGTCTCTCCCAATAGAAGTGATGGTGGACGCGGTGCATAAACTGAAATAT
GAGAACTACACAAGCAGTTTTTTTATAAGAGATATCATCAAGCCCGATCCGCCGAAGAAT
TTGCAACTTAAACCGCTTAAAAACTCACGCCAGGTTGAAGTATCCTGGGAGTATCCGGAT
ACATGGTCAACACCACACAGCTATTTTTCCCTTACCTTCTGTGTGCAGGTCCAAGGGAAG
AGCAAAAGGGAGAAGAAGGACAGGGTATTCACTGATAAAACTTCCGCGACGGTCATCTGC
CGAAAAAACGCTAGTATATCTGTACGGGCGCAGGATAGGTACTATAGTTCTTCTTGGTCT
GAGTGGGCCTCAGTTCCGTGCTCTGGGGGAGGAAGTGGAGGAGGGTCCGGCGGTGGAAGC
GGGGGAGGGAGTCGCAACTTGCCAGTGGCTACACCAGATCCAGGCATGTTTCCATGTCTG
CATCATTCCCAGAATCTCCTGAGAGCGGTGTCAAATATGCTCCAAAAAGCGAGACAAACA
CTGGAATTTTACCCGTGTACCAGTGAGGAGATTGATCACGAGGACATAACCAAGGACAAG
ACCTCAACTGTAGAAGCGTGTTTGCCGCTGGAGTTGACTAAGAATGAGTCCTGCCTCAAT
TCCAGAGAAACTTCATTCATTACTAACGGCAGTTGTCTTGCATCCCGGAAAACGTCCTTT
ATGATGGCCCTTTGCCTTAGTTCAATTTACGAGGATCTTAAAATGTATCAAGTGGAGTTT
AAAACCATGAATGCTAAACTTCTTATGGACCCCAAACGACAAATTTTTCTGGATCAGAAT
ATGCTTGCCGTGATAGACGAACTCATGCAGGCGCTTAATTTTAACTCCGAAACAGTTCCA
CAAAAATCTAGCCTTGAAGAACCTGATTTTTATAAAACGAAGATTAAACTGTGTATCCTG
CTGCATGCCTTTCGCATCCGAGCTGTCACAATCGATAGGGTTATGTCCTACCTTAACGCG
AGCCGGCGCAAGAGGGGTTCCGGAGAGGGAAGGGGTAGTCTGCTCACCTGCGGCGATGTT
GAAGAAAATCCTGGTCCCATGGCGCAAAGTCTGGCTCTTTCACTCCTGATCCTGGTCTTG
GCCTTCGGGATTCCGAGGACCCAAGGAAGTGATGGTGGCGCCCAAGATTGTTGCCTTAAA
TACAGCCAGCGGAAAATACCCGCGAAAGTGGTCAGGAGTTATAGAAAACAGGAGCCTTCC
CTGGGTTGTAGTATCCCCGCCATACTTTTCCTCCCGAGAAAACGGAGCCAGGCCGAACTG
TGCGCTGACCCTAAGGAACTTTGGGTGCAACAACTTATGCAACACCTGGATAAGACACCT
TCTCCTCAAAAGCCAGCTCAGGGCTGCCGAAAAGATAGAGGCGCCTCAAAAACCGGAAAA
AAGGGCAAAGGTTCTAAAGGATGTAAGCGGACTGAACGCTCTCAAACGCCTAAAGGGCCG
taG
IL12_2A_CCL21a (Mouse) (SEQ ID NO: 62)
ATGTGTCCACAGAAGCTGACAATAAGTTGGTTTGCCATTGTCCTCCTGGTGAGCCCACTC
ATGGCAATGTGGGAACTCGAAAAGGATGTCTACGTGGTAGAAGTAGATTGGACTCCAGAC
GCGCCAGGGGAGACAGTGAATTTGACATGTGACACACCAGAAGAAGATGACATTACATGG
ACATCTGACCAACGCCATGGCGTAATAGGGAGTGGGAAAACACTCACGATCACAGTTAAA
GAGTTCTTGGATGCTGGTCAATATACTTGCCATAAAGGCGGCGAGACACTCAGCCACTCA
CATTTGCTTTTGCATAAAAAAGAGAATGGCATTTGGAGCACTGAAATACTTAAGAACTTT
AAGAACAAGACATTTCTCAAGTGTGAGGCCCCTAATTACAGCGGCAGGTTCACGTGCTCA
TGGCTGGTCCAGCGCAACATGGACCTCAAGTTTAACATAAAATCTTCTTCCTCTTCACCT
GACTCCAGAGCTGTTACTTGCGGCATGGCTTCTCTGAGCGCAGAAAAAGTAACGTTGGAT
CAAAGAGACTACGAAAAGTACTCTGTTTCTTGTCAAGAGGATGTTACGTGCCCGACGGCC
GAAGAAACGCTTCCAATTGAACTCGCGTTGGAAGCTCGCCAACAAAACAAGTATGAAAAC
TACAGTACAAGCTTCTTTATACGGGATATAATTAAACCCGATCCCCCCAAGAACTTGCAA
ATGAAACCACTTAAGAACAGCCAGGTGGAAGTTTCCTGGGAGTATCCAGACTCATGGAGT
ACTCCTCACAGCTATTTTTCTCTGAAATTCTTTGTAAGGATACAACGGAAGAAAGAGAAG
ATGAAAGAGACCGAGGAGGGTTGTAATCAGAAGGGAGCGTTTCTCGTGGAGAAAACGTCT
ACCGAAGTCCAATGTAAAGGTGGCAATGTGTGCGTCCAAGCTCAGGATAGATACTATAAT
TCAAGTTGCTCCAAGTGGGCCTGTGTTCCATGCCGCGTTCGGAGCGGGGGAGGTAGCGGA
GGAGGTAGTGGGGGTGGGTCAGGAGGAGGGAGTCGAGTTATCCCGGTGTCAGGCCCCGCA
CGCTGCTTGAGCCAGAGTCGCAACCTCCTTAAGACAACAGATGACATGGTGAAAACAGCA
CGCGAAAAGCTTAAACACTACTCTTGTACGGCGGAGGATATTGATCACGAGGATATTACC
CGAGACCAAACTAGCACTTTGAAAACCTGTCTGCCCCTTGAACTTCATAAAAATGAGAGC
TGTCTGGCTACACGAGAGACGTCAAGTACGACTAGGGGCAGCTGTCTCCCGCCGCAAAAG
ACAAGCCTCATGATGACGCTCTGTTTGGGTTCCATTTACGAGGACTTGAAAATGTATCAA
ACGGAGTTCCAGGCTATAAATGCGGCGTTGCAGAACCATAACCATCAACAAATTATACTT
GATAAAGGCATGTTGGTGGCGATTGATGAACTCATGCAGAGTCTCAATCACAACGGGGAA
ACGTTGAGACAGAAACCCCCAGTCGGTGAAGCGGACCCATATCGAGTAAAAATGAAGCTC
TGCATTCTGCTTCACGCATTCAGCACTAGAGTTGTTACCATCAACCGGGTAATGGGATAT
CTCTCCAGTGCGCGGCGCAAGAGGGGTTCCGGAGAGGGAAGGGGTAGTCTGCTCACCTGC
GGCGATGTTGAAGAAAATCCTGGTCCCATGGCGCAAATGATGACCCTTTCCCTGCTGAGT
CTTGTCCTCGCGCTCTGCATCCCGTGGACGCAGGGGTCTGATGGGGGGGGCCAAGACTGT
TGCCTGAAGTATTCACAAAAAAAGATACCGTACTCTATTGTCAGAGGGTACAGGAAGCAA
GAACCCTCCTTGGGTTGCCCTATACCAGCAATTCTTTTCTCCCCACGCAAGCATTCCAAA
CCAGAACTGTGTGCGAACCCCGAGGAGGGTTGGGTACAGAACTTGATGCGAAGGCTTGAC
CAGCCCCCAGCCCCTGGCAAGCAGTCACCTGGGTGCAGAAAAAACAGAGGTACTTCAAAG
AGCGGCAAGAAAGGCAAAGGGAGTAAAGGATGTAAAAGAACGGAGCAGACCCAGCCTTCA
CGAGGCtaG
IL7 (Mouse) (SEQ ID NO: 64)
ATGTTTCATGTGTCCTTCAGGTACATATTTGGTATCCCACCACTTATATTGGTGCTCTTG
CCTGTAACCAGCTCTGAATGTCATATAAAAGACAAGGAGGGCAAAGCATACGAGTCCGTA
TTGATGATCTCAATCGATGAACTTGACAAGATGACAGGGACCGATTCTAATTGTCCAAAT
AACGAGCCAAACTTCTTTCGGAAACACGTGTGTGATGATACAAAAGAAGCTGCTTTTCTT
AACAGAGCTGCCAGAAAACTCAAGCAGTTCCTCAAGATGAATATATCCGAGGAATTTAAC
GTGCATCTCCTCACAGTATCTCAGGGAACTCAAACCCTTGTAAACTGCACTTCTAAGGAG
GAGAAGAATGTCAAAGAGCAGAAGAAAAATGATGCATGTTTTTTGAAACGGCTGTTGAGG
GAGATCAAAACATGCTGGAATAAAATCCTCAAGGGCTCAATTtaG
IL15 (Human) (SEQ ID NO: 65)
ATGGAAACAGACACATTGCTGCTTTGGGTATTGTTGCTCTGGGTGCCTGGATCAACAGGA
AACTGGGTAAACGTAATTTCAGATCTGAAGAAGATCGAGGACCTTATTCAATCCATGCAC
ATCGATGCCACTCTCTACACCGAAAGCGACGTTCACCCATCTTGCAAGGTGACCGCTATG
AAATGTGAATTGTTGGAACTTCAGGTAATTTCTCTGGAGAGCGGCGATGCCTCAATACAT
GACACCGTTGAAAATCTTATCATCCTTGCTAATGATTCACTCTCTAGTAATGGGAACGTA
ACAGAGAGCGGGTGTAAGGAGTGTGAAGAACTGGAGGAGAAAAACATTAAGGAATTTTTG
CAGTCATTCGTCCATATAGTGCAAATGTTCATAAACACTTCCAGAAGAAAGCGAGGCTCT
GGGGAGGGGCGAGGCTCTCTGCTGACCTGTGGGGATGTAGAAGAGAATCCAGGTCCCATG
GACCGGCTGACCAGCTCATTCCTGCTTCTGATTGTGCCAGCCTACGTGCTCTCCATCACA
TGTCCTCCCCCAATGAGCGTCGAGCATGCTGACATCTGGGTGAAGTCATACTCCTTGTAC
AGCAGAGAGAGATACATTTGTAATTCCGGATTCAAGCGCAAGGCCGGCACCTCCTCTCTG
ACAGAGTGCGTCCTTAACAAAGCAACCAACGTAGCACATTGGACCACACCATCCTTGAAG
TGCATACGAGAACCTAAATCTTGCGATAAGACTCATACTTGTCCACCTTGTCCAGCCCCA
GAACTGCTTGGCGGACCCTCAGTATTTTTGTTCCCACCAAAGCCAAAAGACACACTCATG
ATATCCAGAACTCCTGAGGTGACCTGTGTCGTTGTAGACGTTTCCCACGAAGATCCTGAA
GTAAAATTCAACTGGTACGTGGATGGGGTCGAAGTCCATAACGCCAAGACTAAACCAAGG
GAGGAACAGTATAACTCTACTTACCGAGTAGTTTCTGTGTTGACCGTGCTGCACCAGGAC
TGGTTGAACGGGAAGGAGTACAAATGCAAGGTGAGCAATAAAGCTCTGCCCGCACCAATC
GAAAAGACAATATCTAAGGCCAAGGGGCAGCCACGAGAGCCCCAGGTATACACACTGCCA
CCCTCACGCGATGAATTGACTAAGAACCAGGTTTCCCTGACCTGTCTTGTAAAAGGTTTC
TACCCTTCCGACATAGCTGTTGAGTGGGAAAGTAACGGGCAGCCAGAGAACAATTACAAG
ACAACTCCACCCGTTCTTGATAGCGATGGATCATTTTTTCTGTATTCCAAACTCACTGTC
GATAAAAGTCGCTGGCAGCAAGGCAATGTTTTTAGCTGCTCAGTCATGCACGAAGCACTG
CATAATCACTACACACAAAAAAGTTTGTCCCTTAGCCCTGGTAAGtaG
IL15 (Human) (SEQ ID NO: 66)
ATGTACTCAATGCAGTTGGCCTCCTGTGTAACATTGACCTTGGTCCTCTTGGTCAACAGC
AATTGGATCGATGTACGCTACGACTTGGAGAAGATTGAGTCCCTTATACAGAGTATACAC
ATAGATACAACCTTGTATACTGACAGTGACTTCCATCCCAGCTGTAAAGTGACTGCAATG
AACTGTTTTTTGTTGGAGTTGCAAGTAATTCTGCATGAATACAGCAACATGACCCTCAAT
GAAACCGTTAGGAATGTCCTTTATCTCGCAAATTCTACTCTGAGTAGCAATAAGAATGTT
GCCGAAAGCGGCTGCAAGGAGTGCGAAGAACTGGAGGAAAAAACTTTCACCGAGTTTCTC
CAGAGTTTCATCAGAATTGTCCAAATGTTCATTAATACAAGTAGTGGTGGTGGGAGCGGG
GGTGGAGGCAGTGGGGGAGGTGGGAGCGGAGGTGGAGGGTCCGGAGGGGGGAGCCTTCAA
GGCACTACTTGTCCTCCACCCGTATCCATCGAGCACGCCGATATTCGAGTTAAAAATTAT
AGTGTTAATAGCAGAGAACGATACGTCTGCAACTCAGGGTTTAAGAGAAAGGCCGGAACT
TCAACTCTCATAGAATGCGTGATTAATAAGAATACTAACGTCGCACATTGGACTACTCCC
AGTCTCAAGTGCATACGCGATCCATCTCTCGCTCATTACTCACCAGTACCTACAGTGGTT
ACTCCTAAGGTGACCTCTCAGCCCGAATCACCATCTCCCAGCGCAAAAGAGCCTGAGGCC
TTTTCTCCTAAATCAGACACTGCTATGACTACAGAAACAGCCATAATGCCAGGAAGCCGG
CTGACACCATCTCAAACTACCAGCGCAGGCACAACTGGGACTGGCTCCCACAAAAGCTCA
CGCGCACCAAGTCTCGCCGCAACAATGACATTGGAGCCTACAGCCAGCACATCTCTTAGA
ATCACAGAAATTTCTCCCCACAGTAGCAAGATGACCAAGGTGGCAATTAGTACCAGCGTC
CTTCTTGTAGGAGCTGGAGTTGTGATGGCATTTTTGGCATGGTATATCAAAAGCAGGtaG
IL15 (Mouse) (SEQ ID NO: 67)
ATGAAGATCCTCAAGCCATACATGCGAAACACTAGTATTAGCTGTTACTTGTGTTTTCTG
CTGAATAGTCATTTTTTGACTGAAGCAGGAATCCATGTATTTATACTCGGTTGTGTGTCT
GTAGGTCTGCCAAAGACTGAGGCTAATTGGATTGACGTGCGCTATGATCTTGAAAAAATA
GAGTCCTTGATTCAATCAATACACATCGATACCACTCTCTACACCGACAGTGATTTCCAT
CCTTCCTGCAAGGTAACAGCTATGAATTGCTTCCTCCTGGAGCTCCAAGTCATTCTCCAT
GAGTACTCCAACATGACTTTGAACGAAACTGTAAGAAACGTATTGTATCTGGCTAATAGC
ACCTTGTCTAGTAACAAAAATGTGGCAGAGAGCGGCTGCAAAGAATGTGAAGAATTGGAA
GAGAAAACATTTACAGAGTTCCTGCAATCCTTTATTCGCATCGTCCAAATGTTTATCAAT
ACCTCTtaG
IL15 (Mouse) (SEQ ID NO: 68)
ATGTATTCCATGCAACTTGCCAGTTGTGTAACCCTTACTCTCGTCCTGCTCGTTAATTCC
GCTGGTGCTAACTGGATAGATGTTCGATACGATCTGGAAAAGATTGAGTCCCTTATCCAA
TCCATTCATATAGATACCACCCTTTATACTGACAGCGACTTCCATCCTTCTTGCAAGGTG
ACCGCTATGAATTGTTTCCTGCTGGAACTCCAAGTTATTCTGCATGAATACTCTAATATG
ACACTTAACGAGACCGTAAGAAATGTTCTCTATCTCGCTAATAGTACTTTGAGCTCAAAT
AAGAACGTGGCCGAGTCTGGGTGTAAGGAATGCGAAGAGCTGGAAGAAAAGACATTCACC
GAGTTTCTCCAGTCTTTCATACGGATTGTGCAGATGTTTATCAACACATCAGATTACAAA
GACGACGATGATAAGtaG
IL18 (Mouse) (SEQ ID NO: 69)
ATGGCAGCCATGTCTGAGGACTCTTGTGTGAACTTTAAAGAAATGATGTTCATAGACAAT
ACACTCTACTTTATACCTGAGGAGAATGGAGATTTGGAATCTGACAACTTTGGCAGGCTG
CATTGCACTACCGCAGTTATCCGAAACATCAACGATCAGGTACTGTTTGTTGATAAAAGA
CAACCTGTATTCGAGGACATGACCGACATAGATCAGTCTGCCTCAGAGCCCCAGACTAGG
CTTATCATCTATATGTACAAGGACAGCGAAGTACGAGGCCTGGCTGTTACACTCTCAGTC
AAAGACTCTAAGATGAGCACCCTGTCATGCAAGAACAAAATTATCAGTTTTGAGGAGATG
GACCCACCTGAAAACATAGATGACATTCAGTCAGACCTCATTTTTTTTCAAAAGCGGGTA
CCAGGACACAACAAAATGGAATTTGAATCATCACTCTACGAAGGACATTTCCTTGCATGC
CAGAAAGAGGATGACGCATTCAAATTGATCCTGAAAAAAAAGGACGAAAATGGTGATAAA
TCAGTCATGTTTACATTGACCAATCTTCACCAAAGTtaG
IL18 (Mouse) (SEQ ID NO: 70)
ATGGCTGCAATGTCTGAAGATAGCTGTGTCAACTTTAAGGAGATGATGTTCATTGATAAT
ACTTTGTACTTTATACCTGAAGAAAATGGAGACCTTGAGTCAGACAACTTCGGGAGACTG
CACTGCACAACTGCCGTTATCCGAAACATAAATGATCAAGTATTGTTCGTGGACAAAAGA
CAACCAGTCTTTGAGGATATGACAGACATCGACCAATCCGCATCTGAACCTCAGACTAGG
CTGATCATCTATATGTACGCCGACTCCGAAGTAAGAGGCCTTGCTGTGACACTTAGTGTT
AAGGATAGTAAGATGAGCACACTGTCCTGTAAGAATAAGATTATATCTTTTGAAGAGATG
GACCCTCCCGAGAACATAGATGACATCCAGAGCGACTTGATCTTCTTTCAGAAGCGAGTG
CCAGGCCATAACAAGATGGAATTTGAATCATCTCTTTATGAAGGCCATTTCCTCGCATGT
CAAAAGGAGGACGATGCCTTCAAGCTCATTCTGAAAAAAAAAGACGAGAACGGTGATAAG
AGCGTGATGTTCACTCTGACAAATCTGCACCAGTCAtaG
IL18 (Human) (SEQ ID NO: 71)
ATGTATCGCATGCAACTCCTGTCCTGCATTGCTCTGAGCTTGGCTTTGGTAACCAACTCA
TACTTCGGGAAACTGGAGAGTAAACTCTCCGTAATCAGGAATCTTAATGACCAAGTATTG
TTTATTGACCAGGGCAACCGCCCGTTGTTCGAGGATATGACTGATTCTGACTGTCGGGAT
AACGCTCCGAGAACTATCTTTATCATTTCAATGTACAAGGACAGCCAACCGCGGGGTATG
GCTGTGACAATCAGTGTCAAATGTGAGAAGATTTCCACGCTGTCCTGCGAAAACAAGATA
ATTTCTTTCAAAGAAATGAACCCCCCTGACAATATAAAGGATACAAAGAGTGATATCATC
TTCTTTCAGAGGTCCGTGCCCGGCCACGATAATAAGATGCAATTTGAAAGTTCATCTTAT
GAGGGGTACTTTTTGGCATGCGAGAAAGAAAGGGATCTCTTCAAGTTGATCCTGAAGAAG
GAGGACGAATTGGGCGACCGCTCCATCATGTTCACAGTCCAGAACGAGGACtaG
IL18 (Human) (SEQ ID NO: 72)
ATGTACCGCATGCAGCTCCTGAGTTGTATTGCCCTTTCCCTCGCTCTCGTTACCAATTCT
TACTTCGGTAAGCTTGCCTCTAAACTCTCTGTTATTAGGAACTTGAACGACCAAGTCCTT
TTCATAGACCAAGGGAACAGACCACTGTTTGAAGATATGACGGATAGCGATTGCCGAGAT
AATGCCCCTAGGACGATTTTTATCATTAGTATGTATGCGGACTCTCAACCGAGGGGGATG
GCCGTTACTATAAGTGTGAAATGCGAGAAAATATCAACGCTCAGTTGTGAGAACAAAATC
ATAAGTTTCAAGGAGATGAATCCACCTGATAACATCAAAGACACTAAGTCTGATATTATA
TTTTTCCAACGAAGTGTTCCGGGACACGATAACAAAATGCAATTTGAGAGCTCCTCATAC
GAGGGCTACTTCCTCGCGTGTGAGAAAGAAAGGGATTTGTTTAAGCTTATCCTCAAGAAA
GAGGACGAGTTGGGGGATCGGAGCATAATGTTTACCGTACAGAATGAGGACtaG
IL21 (Mouse) (SEQ ID NO: 73)
ATGGAGCGGACACTCGTGTGTCTTGTCGTAATTTTTCTCGGGACAGTCGCACACAAGTCC
TCACCCCAGGGTCCTGATCGCCTTCTCATACGCCTCCGACATTTGATCGACATTGTAGAG
CAGCTCAAAATTTACGAGAATGACCTCGATCCCGAGCTTTTGAGTGCTCCCCAAGACGTT
AAGGGTCATTGCGAGCACGCAGCTTTTGCTTGCTTCCAGAAGGCCAAGTTGAAACCAAGC
AACCCTGGTAATAATAAGACTTTCATCATCGACTTGGTCGCCCAACTCCGAAGGAGGCTG
CCTGCCCGGCGCGGAGGAAAAAAACAAAAGCATATTGCAAAGTGTCCTTCATGTGATTCA
TACGAAAAGCGGACTCCCAAAGAGTTCTTGGAAAGGTTGAAATGGCTTCTTCAGAAGATG
ATTCATCAACATTTGTCAtaG
IFN-beta (Human) (SEQ ID NO: 74)
ATGACCAACAAATGCCTTTTGCAAATTGCCCTGCTTTTGTGTTTTAGCACTACCGCATTG
AGCATGTCATATAACCTCCTCGGCTTCCTTCAGAGATCATCAAACTTTCAGTGTCAGAAA
CTGCTTTGGCAACTTAATGGCAGGCTCGAATATTGTCTGAAAGATCGGATGAATTTCGAC
ATTCCAGAAGAAATAAAACAGCTTCAACAATTCCAGAAAGAGGACGCCGCCCTGACTATT
TACGAGATGCTCCAGAATATCTTCGCCATTTTCCGGCAGGACAGCTCATCCACGGGGTGG
AATGAGACTATTGTAGAAAATCTTCTGGCTAATGTGTACCATCAAATTAATCACCTCAAA
ACGGTGCTTGAGGAAAAACTTGAAAAGGAAGATTTCACACGGGGCAAGTTGATGTCCTCC
CTGCACCTTAAACGATACTACGGCAGGATTCTTCATTACTTGAAGGCTAAGGAGTATAGC
CATTGCGCGTGGACAATTGTACGGGTAGAAATACTGCGAAACTTTTATTTCATCAACCGG
CTCACTGGATACCTTAGAAATtaG
IFN-beta (Mouse) (SEQ ID NO: 75)
ATGAACAATCGGTGGATACTCCACGCCGCATTTCTCCTCTGCTTTAGCACGACGGCCCTG
TCCATCAACTACAAACAGCTTCAGTTGCAGGAGCGGACTAACATAAGGAAGTGCCAGGAA
CTGCTGGAACAGCTTAATGGTAAAATTAATCTTACATACCGAGCTGACTTCAAAATTCCT
ATGGAAATGACCGAGAAGATGCAGAAATCCTACACGGCATTCGCCATCCAGGAAATGCTC
CAGAACGTATTTCTCGTGTTCCGCAATAATTTCTCTTCTACGGGTTGGAACGAAACCATT
GTTGTTAGACTGCTTGACGAACTGCATCAGCAAACCGTGTTCCTTAAAACCGTGCTTGAG
GAGAAGCAGGAGGAGCGCCTGACTTGGGAGATGTCTAGTACCGCACTTCACTTGAAATCC
TACTACTGGCGCGTTCAGCGGTATCTGAAGCTGATGAAGTATAACTCATACGCCTGGATG
GTAGTGCGCGCAGAGATCTTCAGAAACTTTCTTATCATCCGGCGACTGACCCGAAACTTT
CAGAATtaG
IFN-gamma (Human) (SEQ ID NO: 76)
ATGAAGTACACTAGCTATATATTGGCCTTCCAGCTTTGCATCGTATTGGGTAGCCTCGGA
TGCTATTGCCAAGACCCGTATGTCAAAGAAGCCGAAAATCTCAAAAAGTATTTCAATGCC
GGACACTCAGACGTCGCGGATAACGGTACACTGTTTCTTGGCATCCTGAAAAATTGGAAG
GAAGAGAGTGACAGAAAAATAATGCAGTCACAAATAGTGTCCTTTTACTTTAAGCTGTTC
AAAAATTTCAAGGATGACCAAAGTATCCAGAAGAGTGTTGAAACTATCAAAGAGGACATG
AATGTGAAATTCTTTAACAGTAATAAGAAGAAGCGCGATGACTTCGAGAAACTCACTAAT
TACAGCGTAACGGATCTTAACGTCCAACGCAAGGCAATCCACGAGCTTATACAGGTAATG
GCTGAGCTTAGTCCCGCAGCCAAGACAGGGAAGAGAAAAAGGTCTCAAATGCTTTTTCGG
GGCCGGCGAGCTTCACAAtaG
IFN-gamma (Mouse) (SEQ ID NO: 77)
ATGAACGCTACGCATTGCATCCTCGCACTCCAATTGTTCCTCATGGCTGTGTCAGGGTGT
TACTGTCACGGTACTGTCATAGAAAGCCTCGAATCCCTGAATAACTATTTTAACAGTAGC
GGTATAGATGTAGAAGAAAAGTCTCTCTTTCTTGACATCTGGAGGAATTGGCAAAAGGAT
GGAGACATGAAGATTCTCCAATCTCAGATTATATCATTTTACTTGAGGCTTTTTGAGGTT
CTGAAGGATAACCAGGCGATCAGCAATAATATCAGCGTAATTGAATCTCACCTTATTACA
ACATTTTTCTCAAATTCCAAGGCAAAGAAAGATGCTTTCATGTCTATCGCGAAATTTGAG
GTGAACAATCCTCAGGTACAAAGGCAAGCCTTTAACGAGCTGATTAGAGTTGTACATCAG
TTGTTGCCCGAAAGTAGTCTTAGAAAACGCAAACGGAGCCGATGCtaG
IFN-alpha (Mouse) (SEQ ID NO: 78)
ATGGCAAGGTTGTGCGCTTTTCTCATGGTACTGGCTGTGCTCTCCTATTGGCCTACTTGT
TCTCTGGGATGCGACTTGCCACAGACCCACAATCTGCGGAATAAGAGGGCTCTGACTCTG
CTGGTGCAAATGAGACGGCTCTCTCCACTTAGCTGTTTGAAAGATAGAAAGGATTTCGGG
TTCCCCCAGGAGAAGGTGGATGCCCAGCAGATCAAGAAGGCACAGGCTATCCCCGTCCTT
TCCGAGCTGACCCAGCAAATTTTGAACATCTTTACAAGTAAGGATAGTTCAGCTGCATGG
AATACCACACTTTTGGATTCTTTTTGTAACGATCTGCATCAGCAGCTGAACGATCTCCAG
GGATGCCTGATGCAGCAAGTCGGCGTGCAAGAATTTCCACTCACCCAGGAGGACGCTCTG
CTCGCAGTGCGAAAGTATTTTCACCGAATTACCGTGTACCTCCGGGAGAAAAAGCATTCA
CCCTGCGCTTGGGAAGTAGTCAGGGCCGAAGTATGGAGAGCCCTTAGTAGCTCCGCTAAT
GTACTGGGCCGGTTGCGGGAAGAGAAAtaG
IL-7 (SEQ ID NO: 393)
GACTGTGATATCGAGGGCAAAGACGGCAAGCAGTACGAGAGCGTGCTGATGGTGTCCATC
GACCAGCTGCTGGACAGCATGAAGGAAATCGGCAGCAACTGCCTGAACAACGAGTTCAAC
TTCTTCAAGCGGCACATCTGCGACGCCAACAAAGAAGGCATGTTCCTGTTCAGAGCCGCC
AGAAAGCTGCGGCAGTTCCTGAAGATGAACAGCACCGGCGACTTCGACCTGCATCTGCTG
AAAGTGTCTGAGGGCACCACCATCCTGCTGAATTGCACCGGCCAAGTGAAGGGCAGAAAG
CCTGCTGCTCTGGGAGAAGCCCAGCCTACCAAGAGCCTGGAAGAGAACAAGTCCCTGAAA
GAGCAGAAGAAGCTGAACGACCTCTGCTTCCTGAAGCGGCTGCTGCAAGAGATCAAGACC
TGCTGGAACAAGATCCTGATGGGCACCAAAGAGCAC

The first engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 326. The first engineered nucleic acid can include a nucleotide sequence having the sequence shown in SEQ ID NO: 326.

The first engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 310. The first engineered nucleic acid can include a nucleotide sequence having the sequence shown in SEQ ID NO: 310.

The first engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 327. The first engineered nucleic acid can include a nucleotide sequence having the sequence shown in SEQ ID NO: 327.

The first engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 314. The first engineered nucleic acid can include a nucleotide sequence having the sequence shown in SEQ ID NO: 314.

The first engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 315. The first engineered nucleic acid can include a nucleotide sequence having the sequence shown in SEQ ID NO: 315.

The second engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 317. The second engineered nucleic acid can include a nucleotide sequence having the sequence shown in SEQ ID NO: 317.

The second engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 318. The second engineered nucleic acid can include a nucleotide sequence having the sequence shown in SEQ ID NO: 318.

The first engineered nucleic acid can include a nucleotide sequence having the sequence shown in SEQ ID NO: 310; and (b) the second engineered nucleic acid can include a nucleotide sequence having the sequence shown in SEQ ID NO: 317.

The first engineered nucleic acid can include a nucleotide sequence having the sequence shown in SEQ ID NO: 327; and (b) the second engineered nucleic acid can include a nucleotide sequence having the sequence shown in SEQ ID NO: 317.

The first engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 310; and (b) the second engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 317.

The first engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 327; and (b) the second engineered nucleic acid can include a nucleotide sequence at least 90%, at least 91%, at least 92%, at least 93%, at least 94%, at least 95%, at least 96%, at least 97%, at least 98%, or at least 99% identical to SEQ ID NO: 317.

Immunoresponsive cells provided for herein can include any one of the engineered nucleic acids described herein. Immunoresponsive cells provided for herein can include combinations of any one of the engineered nucleic acids described herein. Immunoresponsive cells provided for herein can include two or more of any one of the engineered nucleic acids described herein.

Immunoresponsive cells provided for herein can include a nucleotide sequence at least 90%, at least 91%, at least 92%, at least 93%, at least 94%, at least 95%, at least 96%, at least 97%, at least 98%, or at least 99% identical to SEQ ID NO: 309. Immunoresponsive cells provided for herein can include a nucleotide sequence having the sequence shown in SEQ ID NO: 309.

Immunoresponsive cells provided for herein can include a nucleotide sequence at least 90%, at least 91%, at least 92%, at least 93%, at least 94%, at least 95%, at least 96%, at least 97%, at least 98%, or at least 99% identical to SEQ ID NO: 326. Immunoresponsive cells provided for herein can include a nucleotide sequence having the sequence shown in SEQ ID NO: 326.

Immunoresponsive cells provided for herein can include a nucleotide sequence at least 90%, at least 91%, at least 92%, at least 93%, at least 94%, at least 95%, at least 96%, at least 97%, at least 98%, or at least 99% identical to SEQ ID NO: 310. Immunoresponsive cells provided for herein can include a nucleotide sequence having the sequence shown in SEQ ID NO: 310.

Immunoresponsive cells provided for herein can include a nucleotide sequence at least 90%, at least 91%, at least 92%, at least 93%, at least 94%, at least 95%, at least 96%, at least 97%, at least 98%, or at least 99% identical to SEQ ID NO: 327. Immunoresponsive cells provided for herein can include a nucleotide sequence having the sequence shown in SEQ ID NO: 327.

Immunoresponsive cells provided for herein can include a nucleotide sequence at least 90%, at least 91%, at least 92%, at least 93%, at least 94%, at least 95%, at least 96%, at least 97%, at least 98%, or at least 99% identical to SEQ ID NO: 314. Immunoresponsive cells provided for herein can include a nucleotide sequence having the sequence shown in SEQ ID NO: 314.

Immunoresponsive cells provided for herein can include a nucleotide sequence at least 90%, at least 91%, at least 92%, at least 93%, at least 94%, at least 95%, at least 96%, at least 97%, at least 98%, or at least 99% identical to SEQ ID NO: 315. Immunoresponsive cells provided for herein can include a nucleotide sequence having the sequence shown in SEQ ID NO: 315.

Immunoresponsive cells provided for herein can include a nucleotide sequence at least 90%, at least 91%, at least 92%, at least 93%, at least 94%, at least 95%, at least 96%, at least 97%, at least 98%, or at least 99% identical to SEQ ID NO: 317. Immunoresponsive cells provided for herein can include a nucleotide sequence having the sequence shown in SEQ ID NO: 317.

Immunoresponsive cells provided for herein can include a nucleotide sequence at least 90%, at least 91%, at least 92%, at least 93%, at least 94%, at least 95%, at least 96%, at least 97%, at least 98%, or at least 99% identical to SEQ ID NO: 318. Immunoresponsive cells provided for herein can include a nucleotide sequence having the sequence shown in SEQ ID NO: 318.

Immunoresponsive cells provided for herein can include a first engineered nucleic acid including a nucleotide sequence having the sequence shown in SEQ ID NO: 310; and (b) a second engineered nucleic acid including a nucleotide sequence having the sequence shown in SEQ ID NO: 317.

Immunoresponsive cells provided for herein can include a first engineered nucleic acid including a nucleotide sequence having the sequence shown in SEQ ID NO: 327; and (b) a second engineered nucleic acid including a nucleotide sequence having the sequence shown in SEQ ID NO: 317.

Immunoresponsive cells provided for herein can include a first engineered nucleic acid including a nucleotide sequence at least 90%, at least 91%, at least 92%, at least 93%, at least 94%, at least 95%, at least 96%, at least 97%, at least 98%, or at least 99% identical to SEQ ID NO: 310; and (b) a second engineered nucleic acid including a nucleotide sequence at least 90%, at least 91%, at least 92%, at least 93%, at least 94%, at least 95%, at least 96%, at least 97%, at least 98%, or at least 99% identical to SEQ ID NO: 317.

Immunoresponsive cells provided for herein can include a first engineered nucleic acid including a nucleotide sequence at least 90%, at least 91%, at least 92%, at least 93%, at least 94%, at least 95%, at least 96%, at least 97%, at least 98%, or at least 99% identical to SEQ ID NO: 327; and (b) a second engineered nucleic acid including a nucleotide sequence at least 90%, at least 91%, at least 92%, at least 93%, at least 94%, at least 95%, at least 96%, at least 97%, at least 98%, or at least 99% identical to SEQ ID NO: 317.

Expression vectors provided for herein can include any one of the engineered nucleic acids described herein. Expression vectors provided for herein can include combinations of any one of the engineered nucleic acids described herein. Expression vectors provided for herein can include two or more of any one of the engineered nucleic acids described herein.

Expression vectors provided for herein can include a nucleotide sequence at least 90%, at least 91%, at least 92%, at least 93%, at least 94%, at least 95%, at least 96%, at least 97%, at least 98%, or at least 99% identical to SEQ ID NO: 309. Expression vectors provided for herein can include a nucleotide sequence having the sequence shown in SEQ ID NO: 309.

Expression vectors provided for herein can include a nucleotide sequence at least 90%, at least 91%, at least 92%, at least 93%, at least 94%, at least 95%, at least 96%, at least 97%, at least 98%, or at least 99% identical to SEQ ID NO: 326. Expression vectors provided for herein can include a nucleotide sequence having the sequence shown in SEQ ID NO: 326.

Expression vectors provided for herein can include a nucleotide sequence at least 90%, at least 91%, at least 92%, at least 93%, at least 94%, at least 95%, at least 96%, at least 97%, at least 98%, or at least 99% identical to SEQ ID NO: 310. Expression vectors provided for herein can include a nucleotide sequence having the sequence shown in SEQ ID NO: 310.

Expression vectors provided for herein can include a nucleotide sequence at least 90%, at least 91%, at least 92%, at least 93%, at least 94%, at least 95%, at least 96%, at least 97%, at least 98%, or at least 99% identical to SEQ ID NO: 327. Expression vectors provided for herein can include a nucleotide sequence having the sequence shown in SEQ ID NO: 327.

Expression vectors provided for herein can include a nucleotide sequence at least 90%, at least 91%, at least 92%, at least 93%, at least 94%, at least 95%, at least 96%, at least 97%, at least 98%, or at least 99% identical to SEQ ID NO: 314. Expression vectors provided for herein can include a nucleotide sequence having the sequence shown in SEQ ID NO: 314.

Expression vectors provided for herein can include a nucleotide sequence at least 90%, at least 91%, at least 92%, at least 93%, at least 94%, at least 95%, at least 96%, at least 97%, at least 98%, or at least 99% identical to SEQ ID NO: 315. Expression vectors provided for herein can include a nucleotide sequence having the sequence shown in SEQ ID NO: 315.

Expression vectors provided for herein can include a nucleotide sequence at least 90%, at least 91%, at least 92%, at least 93%, at least 94%, at least 95%, at least 96%, at least 97%, at least 98%, or at least 99% identical to SEQ ID NO: 317. Expression vectors provided for herein can include a nucleotide sequence having the sequence shown in SEQ ID NO: 317.

Expression vectors provided for herein can include a nucleotide sequence at least 90%, at least 91%, at least 92%, at least 93%, at least 94%, at least 95%, at least 96%, at least 97%, at least 98%, or at least 99% identical to SEQ ID NO: 318. Expression vectors provided for herein can include a nucleotide sequence having the sequence shown in SEQ ID NO: 318.

Expression vectors provided for herein can include a first engineered nucleic acid including a nucleotide sequence having the sequence shown in SEQ ID NO: 310; and (b) a second engineered nucleic acid including a nucleotide sequence having the sequence shown in SEQ ID NO: 317.

Expression vectors provided for herein can include a first engineered nucleic acid including a nucleotide sequence having the sequence shown in SEQ ID NO: 327; and (b) a second engineered nucleic acid including a nucleotide sequence having the sequence shown in SEQ ID NO: 317.

Expression vectors provided for herein can include a first engineered nucleic acid including a nucleotide sequence at least 90%, at least 91%, at least 92%, at least 93%, at least 94%, at least 95%, at least 96%, at least 97%, at least 98%, or at least 99% identical to SEQ ID NO: 310; and (b) a second engineered nucleic acid including a nucleotide sequence at least 90%, at least 91%, at least 92%, at least 93%, at least 94%, at least 95%, at least 96%, at least 97%, at least 98%, or at least 99% identical to SEQ ID NO: 317.

Expression vectors provided for herein can include a first engineered nucleic acid including a nucleotide sequence at least 90%, at least 91%, at least 92%, at least 93%, at least 94%, at least 95%, at least 96%, at least 97%, at least 98%, or at least 99% identical to SEQ ID NO: 327; and (b) a second engineered nucleic acid including a nucleotide sequence at least 90%, at least 91%, at least 92%, at least 93%, at least 94%, at least 95%, at least 96%, at least 97%, at least 98%, or at least 99% identical to SEQ ID NO: 317.

### Secretion Signals and Signal-Anchors

The one or more effector molecules (e.g., any of the cytokines described herein) of the membrane-cleavable chimeric proteins provided for herein are in general secretable effector molecules having a secretion signal peptide (also referred to as a signal peptide or signal sequence) at the chimeric protein's N-terminus (e.g., an effector molecule's N-terminus for S-C-MT) that direct newly synthesized proteins destined for secretion or membrane localization (also referred to as membrane insertion) to the proper protein processing pathways. For chimeric proteins having the formula MT-C-S, a membrane tethering domain generally has a signal-anchor sequence (e.g., signal-anchor sequences of a Type II transmembrane protein) that direct newly synthesized proteins destined for membrane localization to the proper protein processing pathways. For chimeric proteins having the formula S-C-MT, a membrane tethering domain having a reverse signal-anchor sequence (e.g., signal-anchor sequences of certain Type III transmembrane proteins) can be used, generally without a separate secretion signal peptide, that direct newly synthesized proteins destined for membrane localization to the proper protein processing pathways.

In general, for all membrane-cleavable chimeric proteins described herein, the one or more effector molecules are secretable effector molecules (referred to as “S” in the formula S-C-MT or MT-C-S). In embodiments with two or more chimeric proteins, each chimeric protein can comprise a secretion signal. In embodiments with two or more chimeric proteins, each chimeric protein can comprise a secretion signal such that each effector molecule is capable of secretion from an engineered cell following cleavage of the protease cleavage site.

The secretion signal peptide operably associated with an effector molecule can be a native secretion signal peptide (e.g., the secretion signal peptide generally endogenously associated with the given effector molecule, such as a cytokine's endogenous secretion signal peptide). The secretion signal peptide operably associated with an effector molecule can be a non-native secretion signal peptide native secretion signal peptide. Non-native secretion signal peptides can promote improved expression and function, such as maintained secretion, in particular environments, such as tumor microenvironments. Non-limiting examples of non-native secretion signal peptide are shown in Table 3.

TABLE 3
Exemplary Signal Secretion Peptides

| | | Source | |
|---|---|---|---|
| Name | Protein SEQUENCE | (Uniprot) | DNA SEQUENCE |
| IL12 | MCHQQLVISWFSL | P29460 | ATGTGTCACCAGCAGCTCGTTATATC |
| | VFLASPLVA (SEQ | | CTGGTTTAGTTTGGTGTTTCTCGCTTC |
| | ID NO: 112) | | ACCCCTGGTGGCA (SEQ ID NO: 31) |
| IL12 (Codon | MCHQQLVISWFSL | — | ATGTGCCATCAGCAACTCGTCATCTC |
| Optimized) | VFLASPLVA (SEQ | | CTGGTTCTCCCTTGTGTTCCTCGCTTC |
| | ID NO: 112) | | CCCTCTGGTCGCC (SEQ ID NO: 32) |
| IL2 (Optimized) | MQLLSCIALILALV | — | ATGCAACTGCTGTCATGTATCGCACT |
| | (SEQ ID NO: 113) | | CATCCTGGCGCTGGTA (SEQ ID NO: |
| | | | 33) |
| IL2 (Native) | MYRMQLLSCIALSL | P60568 | ATGTATCGGATGCAACTTTTGAGCTG |
| | ALVINS (SEQ ID | | CATCGCATTGTCTCTGGCGCTGGTGA |
| | NO: 114) | | CAAATTCC (SEQ ID NO: 34) |
| Trypsinogen-2 | MNLLLILTFVAAAV | P07478 | ATGAATCTCTTGCTCATACTTACGTT |
| | A (SEQ ID NO: 115) | | TGTCGCTGCTGCCGTTGCG (SEQ ID |
| | | | NO: 35) |
| Gaussia | MGVKVLFALICIAV | — | ATGGGCGTGAAGGTCTTGTTTGCCCT |
| Luciferase | AEA (SEQ ID NO: | | TATCTGCATAGCTGTTGCGGAGGCG |
| | 116) | | (SEQ ID NO: 36) |
| CD5 | MPMGSLQPLATLY | P06127 | ATGCCGATGGGGAGCCTTCAACCTTT |
| | LLGMLVASCLG | | GGCAACGCTTTATCTTCTGGGGATGT |
| | (SEQ ID NO: 117) | | TGGTTGCTAGTTGCCTTGGG (SEQ ID |
| | | | NO: 37) |
| IgKVII (mouse) | METDTLLLWVLLL | | ATGGAAACTGACACGTTGTTGCTGTG |
| | WVPGSTGD (SEQ | | GGTATTGCTCTTGTGGGTCCCAGGAT |
| | ID NO: 118) | | CTACGGGCGAC (SEQ ID NO: 38) |
| IgKVII (human) | MDMRVPAQLLGLL | P01597 | ATGGATATGAGGGTTCCCGCCCAGCT |
| | LLWLRGARC (SEQ | | TTTGGGGCTGCTTTTGTTGTGGCTTC |
| | ID NO: 119) | | GAGGGGCTCGGTGT (SEQ ID NO: 39) |
| VSV-G | MKCLLYLAFLFIGV | — | ATGAAGTGTCTGTTGTACCTGGCGTT |
| | NC (SEQ ID NO: 120) | | TCTGTTCATTGGTGTAAACTGT (SEQ |
| | | | ID NO: 40) |
| Prolactin | MNIKGSPWKGSLL | P01236 | ATGAATATCAAAGGAAGTCCGTGGA |
| | LLLVSNLLLCQSVA | | AGGGTAGTCTCCTGCTGCTCCTCGTA |
| | P (SEQ ID NO: 121) | | TCTAACCTTCTCCTTTGTCAATCCGT |
| | | | GGCACCC (SEQ ID NO: 41) |
| Serum albumin | MKWVTFISLLFLFS | P02768 | ATGAAATGGGTAACATTCATATCACT |
| preproprotein | SAYS (SEQ ID NO: | | TCTCTTTCTGTTCAGCTCTGCGTATTC |
| | 122) | | T (SEQ ID NO: 42) |
| Azurocidin | MTRLTVLALLAGL | 20160 | ATGACAAGGCTTACTGTTTTGGCTCT |
| preproprotein | LASSRA (SEQ ID | | CCTCGCTGGACTCTTGGCTTCCTCCC |
| | NO:123) | | GAGCA (SEQ ID NO: 43) |
| Osteonectin | MRAWIFFLLCLAG | P09486 | ATGAGGGCTTGGATTTTTTTTCTGCT |
| (BM40) | RALA (SEQ ID NO: | | CTGCCTTGCCGGTCGAGCCCTGGCG |
| | 124) | | (SEQ ID NO: 44) |
| CD33 | MPLLLLLPLLWAG | P20138 | ATGCCTCTTCTGCTTTTGCTTCCTCTT |
| | ALA (SEQ ID NO: | | TTGTGGGCAGGTGCCCTCGCA (SEQ |
| | 125) | | ID NO: 45) |
| IL6 | MNSFSTSAFGPVAF | P05231 | ATGAACTCTTTCTCAACCTCTGCGTT |
| | SLGLLLVLPAAFPA | | TGGTCCGGTCGCTTTCTCCCTTGGGC |
| | P (SEQ ID NO: 126) | | TCCTGCTTGTCTTGCCAGCAGCGTTT |
| | | | CCTGCGCCA (SEQ ID NO: 46) |
| IL8 | MTSKLAVALLAAF | P10145 | ATGACAAGTAAACTGGCGGTAGCCT |
| | LISAALC (SEQ ID | | TGCTCGCGGCCTTTTTGATTTCCGCA |
| | NO: 127) | | GCCCTTTGT (SEQ ID NO: 47) |
| CCL2 | MKVSAALLCLLLIA | P13500 | ATGAAGGTAAGTGCAGCGTTGCTTTG |
| | ATFIPQGLA (SEQ | | CCTTCTCCTCATTGCAGCGACCTTTA |
| | ID NO: 128) | | TTCCTCAAGGGCTGGCC (SEQ ID NO: |
| | | | 48) |
| TIMP2 | MGAAARTLRLALG | P16035 | ATGGGAGCGGCAGCTAGAACACTTC |
| | LLLLATLLRPADA | | GACTTGCCCTTGGGCTCTTGCTCCTT |
| | (SEQ ID NO: 129) | | GCAACCCTCCTTAGACCTGCCGACGC |
| | | | A (SEQ ID NO: 49) |
| VEGFB | MSPLLRRLLLAALL | P49765 | ATGTCACCGTTGTTGCGGAGATTGCT |
| | QLAPAQA (SEQ ID | | GTTGGCCGCACTTTTGCAACTGGCTC |
| | NO: 130) | | CTGCTCAAGCC (SEQ ID NO: 50) |
| Osteoprotegerin | MNNLLCCALVFLDI | O00300 | ATGAATAACCTGCTCTGTTGTGCGCT |
| | SIKWTTQ (SEQ ID | | CGTGTTCCTGGACATTTCTATAAAAT |
| | NO: 131) | | GGACAACGCAA (SEQ ID NO: 51) |
| Serpin E1 | MQMSPALTCLVLG | P05121 | ATGCAAATGTCTCCTGCCCTTACCTG |
| | LALVFGEGSA (SEQ | | TCTCGTACTTGGTCTTGCGCTCGTAT |
| | ID NO: 132) | | TTGGAGAGGGATCAGCC (SEQ ID NO: |
| | | | 52) |
| GROalpha | MARAALSAAPSNP | P09341 | ATGGCAAGGGCTGCACTCAGTGCTG |
| | RLLRVALLLLLLVA | | CCCCGTCTAATCCCAGATTGCTTCGA |
| | AGRRAAG (SEQ ID | | GTTGCATTGCTTCTTCTGTTGCTGGTT |
| | NO: 133) | | GCAGCTGGTAGGAGAGCAGCGGGT |
| | | | (SEQ ID NO: 53) |
| CXCL12 | MNAKVVVVLVLVL | P48061 | ATGAATGCAAAAGTCGTGGTCGTGC |
| | TALCLSDG (SEQ ID | | TGGTTTTGGTTCTGACGGCGTTGTGT |
| | NO: 134) | | CTTAGTGATGGG (SEQ ID NO: 54) |
| IL21 (Codon | MERIVICLMVIFLG | Q9HBE4 | ATGGAACGCATTGTGATCTGCCTGAT |
| Optimized) | TLVHK SSS (SEQ ID | | GGTCATCTTCCTGGGCACCTTAGTGC |
| | NO: 135) | | ACAAGTCGAGCAGC (SEQ ID NO: 55) |
| CD8 | MALPVTALLLPLAL | — | ATGGCCTTACCAGTGACCGCCTTGCT |
| | LLHAARP (SEQ ID | | CCTGCCGCTGGCCTTGCTGCTCCACG |
| | NO: 136) | | CCGCCAGGCCG (SEQ ID NO: 139) |
| CD8 (Codon | MALPVTALLLPLAL | — | ATGGCGCTCCCGGTGACAGCACTTCT |
| Optimized) | LLHAARP (SEQ ID | | CTTGCCTCTTGCCCTGCTGTTGCATG |
| | NO: 137) | | CCGCGCGCCCA (SEQ ID NO: 140) |
| GMCSFRa | MLLVTSLLLCELPH | — | ATGTTGCTCGTGACATCCCTCTTGCT |
| | PAFLLIP (SEQ ID | | TTGTGAGTTGCCTCATCCCGCATTCC |
| | NO: 138) | | TGCTCATCCCA (SEQ ID NO: 141) |
| GM-CSFRa | MLLLVTSLLLCELP | | ATGCTGCTGCTGGTCACATCTCTGCT |
| | HPAFLLIP (SEQ ID | | GCTGTGCGAGCTGCCCCATCCTGCCT |
| | NO: 216) | | TTCTGCTGATCCCT (SEQ ID NO: 217) |
| NKG2D | PFFFCCFIAVAMGIR | | CCCTTCTTCTTCTGTTGCTTTATCGCC |
| | FIIMVA (SEQ ID | | GTGGCCATGGGCATCCGCTTCATCAT |
| | NO: 192) | | TATGGTGGCC (SEQ ID NO: 193) |
| IgE | MDWTWILFLVAAA | | ATGGACTGGACCTGGATCCTGTTTCT |
| | TRVHS (SEQ ID NO: | | GGTGGCCGCTGCCACAAGAGTGCAC |
| | 218) | | AGC (SEQ ID NO: 214) |

### Protease Cleavage Site

In general, all membrane-cleavable chimeric proteins described herein contain a protease cleavage site (referred to as “C” in the formula S-C-MT or MT-C-S). In general, the protease cleavage site can be any amino acid sequence motif capable of being cleaved by a protease. Examples of protease cleavage sites include, but are not limited to, a Type 1 transmembrane protease cleavage site, a Type II transmembrane protease cleavage site, a GPI anchored protease cleavage site, an ADAM8 protease cleavage site, an ADAM9 protease cleavage site, an ADAM10 protease cleavage site, an ADAM12 protease cleavage site, an ADAM15 protease cleavage site, an ADAM17 protease cleavage site, an ADAM19 protease cleavage site, an ADAM20 protease cleavage site, an ADAM21 protease cleavage site, an ADAM28 protease cleavage site, an ADAM30 protease cleavage site, an ADAM33 protease cleavage site, a BACE1 protease cleavage site, a BACE2 protease cleavage site, a SIP protease cleavage site, an MT1-MMP protease cleavage site, an MT3-MMP protease cleavage site, an MT5-MMP protease cleavage site, a furin protease cleavage site, a PCSK7 protease cleavage site, a matriptase protease cleavage site, a matriptase-2 protease cleavage site, an MMP9 protease cleavage site, or an NS3 protease cleavage site.

One example of a protease cleavage site is a hepatitis C virus (HCV) nonstructural protein 3 (NS3) protease cleavage site, including, but not limited to, a NS3/NS4A, a NS4A/NS4B, a NS4B/NS5A, or a NS5A/NS5B cleavage site. For a description of NS3 protease and representative sequences of its cleavage sites for various strains of HCV, see, e.g., Hepatitis C Viruses: Genomes and Molecular Biology (S. L. Tan ed., Taylor & Francis, 2006), Chapter 6, pp. 163-206; herein incorporated by reference in its entirety. For example, the sequences of HCV NS4A/4B protease cleavage site; HCV NS5A/5B protease cleavage site; C-terminal degron with NS4A/4B protease cleavage site; N-terminal degron with HCV NS5A/5B protease cleavage site are provided. Representative NS3 sequences are listed in the National Center for Biotechnology Information (NCBI) database. See, for example, NCBI entries: Accession Nos. YP 001491553, YP_001469631, YP_001469632, NP 803144, NP_671491, YP_001469634, YP 001469630, YP_001469633, ADA68311, ADA68307, AFP99000, AFP98987, ADA68322, AFP99033, ADA68330, AFP99056, AFP99041, CBF60982, CBF60817, AHH29575, AIZ00747, AIZ00744, ABI36969, ABN05226, KF516075, KF516074, KF516056, AB826684, AB826683, JX171009, JX171008, JX171000, EU847455, EF154714, GU085487, JX171065, JX171063; all of which sequences (as entered by the date of filing of this application) are herein incorporated by reference.

Another example of a protease cleavage site is an ADAM17-specific protease (also referred to as Tumor Necrosis Factor-α Converting Enzyme [TACE]) cleavage site. An ADAM17-specific protease cleavage site can be an endogenous sequence of a substrate naturally cleaved by ADAM17. An ADAM17-specific protease cleavage site can be an engineered sequence capable of being cleaved by ADAM17. An engineered ADAM17-specific protease cleavage site can be an engineered for specific desired properties including, but not limited to, optimal expression of the chimeric proteins, specificity for ADAM17, rate-of-cleavage by ADAM17, ratio of secreted and membrane-bound chimeric protein levels, and cleavage in different cell states. A protease cleavage site can be selected for specific cleavage by ADAM17. For example, certain protease cleavage sites capable of being cleaved by ADAM17 are also capable of cleavage by additional ADAM family proteases, such as ADAM10. Accordingly, an ADAM17-specific protease cleavage site can be selected and/or engineered such that cleavage by other proteases, such as ADAM10, is reduced or eliminated. A protease cleavage site can be selected for rate-of-cleavage by ADAM17. For example, it can be desirable to select a protease cleavage site demonstrating a specific rate-of-cleavage by ADAM17, such as reduced cleavage kinetics relative to an endogenous sequence of a substrate naturally cleaved by ADAM17. In such cases, in general, a specific rate-of-cleavage can be selected to regulate the rate of processing of the chimeric protein, which in turn regulates the rate of release/secretion of the payload effector molecule. Accordingly, an ADAM17-specific protease cleavage site can be selected and/or engineered such that the sequence demonstrates a desired rate-of-cleavage by ADAM17. A protease cleavage site can be selected for both specific cleavage by ADAM17 and rate-of-cleavage by ADAM17. Exemplary ADAM17-specific protease cleavage sites, including those demonstrating particular specificity and rate-of-cleavage kinetics, are shown in Table 4A below with reference to the site of cleavage (P5-P1: N-terminal; P1′-P5′: C-terminal). Further details of ADAM17 and ADAM10, including expression and protease cleavage sites, are described in Sharma, et al. (J Immunol Oct. 15, 2017, 199 (8) 2865-2872), Pham et al. (Anticancer Res. 2017 October;37(10):5507-5513), Caescu et al. (Biochem J. 2009 Oct. 23; 424(1): 79-88), and Tucher et al. (J. Proteome Res. 2014, 13, 4, 2205-2214), each herein incorporated by reference for purposes.

TABLE 4A
Potential ADAM17 Protease Cleavage Site Sequences

| | | | | | | | | | | | SEQ |
|---|---|---|---|---|---|---|---|---|---|---|---|
| | | | | | | | | | | | ID |
| P5 | P4 | P3 | P2 | P1 | P1′ | P2′ | P3′ | P4′ | P5′ | FULL SEQ | NO |
| P | R | A | E | A | V | K | G | G | | PRAEAVKGG | 179 |
| P | R | A | E | A | L | K | G | G | | PRAEALKGG | 180 |
| P | R | A | E | Y | S | K | G | G | | PRAEYSKGG | 181 |
| P | R | A | E | P | I | K | G | G | | PRAEPIKGG | 182 |
| P | R | A | E | A | Y | K | G | G | | PRAEAYKGG | 183 |
| P | R | A | E | S | S | K | G | G | | PRAESSKGG | 184 |
| P | R | A | E | F | T | K | G | G | | PRAEFTKGG | 185 |
| D | E | P | H | Y | S | Q | R | R | | DEPHYSQRR | 187 |
| P | P | L | G | P | I | F | N | P | G | PPLGPIFNPG | 188 |
| P | L | A | Q | A | Y | R | S | S | | PLAQAYRSS | 189 |
| T | P | I | D | S | S | F | N | P | D | TPIDSSFNPD | 190 |
| V | T | P | E | P | I | F | S | L | I | VTPEPIFSLI | 191 |
| P | R | A | E | A | A | K | G | G | | PRAEAAKGG | 186 |

In some embodiments, the protease cleavage site comprises a first region having the amino acid sequence of PRAE (SEQ ID NO: 176). In some embodiments, the protease cleavage site comprises a second region having the amino acid sequence of KGG (SEQ ID NO: 177). In some embodiments, the first region is located N-terminal to the second region. In some embodiments, the protease cleavage site comprises the amino acid sequence of PRAEX₁X₂KGG (SEQ ID NO: 178), wherein X₁ is A, Y, P, S, or F, and wherein X₂ is V, L, S, I, Y, T, or A. In some embodiments, the protease cleavage site comprises the amino acid sequence of PRAEAVKGG (SEQ ID NO: 179). In some embodiments, the protease cleavage site comprises the amino acid sequence of PRAEALKGG (SEQ ID NO: 180). In some embodiments, the protease cleavage site comprises the amino acid sequence of PRAEYSKGG (SEQ ID NO: 181). In some embodiments, the protease cleavage site comprises the amino acid sequence of PRAEPIKGG (SEQ ID NO: 182). In some embodiments, the protease cleavage site comprises the amino acid sequence of PRAEAYKGG (SEQ ID NO: 183). In some embodiments, the protease cleavage site comprises the amino acid sequence of PRAESSKGG (SEQ ID NO: 184). In some embodiments, the protease cleavage site comprises the amino acid sequence of PRAEFTKGG (SEQ ID NO: 185). In some embodiments, the protease cleavage site comprises the amino acid sequence of PRAEAAKGG (SEQ ID NO: 186). In some embodiments, the protease cleavage site comprises the amino acid sequence of DEPHYSQRR (SEQ ID NO: 187). In some embodiments, the protease cleavage site comprises the amino acid sequence of G (SEQ ID NO: 188). In some embodiments, the protease cleavage site comprises the amino acid sequence of PLAQAYRSS (SEQ ID NO: 189). In some embodiments, the protease cleavage site comprises the amino acid sequence of TPIDSSFNPD (SEQ ID NO: 190). In some embodiments, the protease cleavage site comprises the amino acid sequence of VTPEPIFSLI (SEQ ID NO: 191).

In certain embodiments, a cleavage site comprises a linker sequence. A cleavage site may be flanked on the N terminal and/or C terminal sides by a linker sequence. For example and without limitation, the cleavage site may be flanked on both the N terminal and C terminal sides by a partial glycine-serine (GS) linker sequence. Upon cleavage, the N terminal partial GS linker, and C terminal partial GS linker, join to form a GS linker sequence, such as SEQ ID NO: 215.

In certain embodiments, the cleavage site and linker comprise the amino acid sequence of SGGGGSGGGGSGVTPEPIFSLIGGGSGGGGSGGGSLQ (SEQ ID NO: 287). An exemplary nucleic acid sequence encoding SEQ ID NO: 287 is TCTGGCGGCGGAGGATCTGGCGGAGGTGGAAGCGGAGTTACACCCGAGCCTATCTT CAGCCTGATCGGAGGCGGTAGCGGAGGCGGAGGAAGTGGTGGCGGATCTCTGCAA (SEQ ID NO: 288). In some embodiments, nucleic acids encoding SEQ ID NO: 287 may comprise SEQ ID NO: 288, or a nucleic acid sequence that is at least 90%, at least 91%, at least 92%, at least 93%, at least 94%, at least 95%, at least 96%, at least 97%, at least 98%, or at least 99% identical to SEQ ID NO: 288.

In certain embodiments, the protease cleavage site is N-terminal to a linker. In certain embodiments, the protease cleavage site and linker comprise the amino acid sequence of PRAEALKGGSGGGGSGGGGSGGGGSGGGGSGGGSLQ (SEQ ID NO: 289). An exemplary nucleic acid sequence encoding SEQ ID NO: 289 is CCCAGAGCCGAGGCTCTGAAAGGCGGATCAGGCGGCGGTGGTAGTGGAGGCGGAG GCTCAGGCGGCGGAGGTTCCGGAGGTGGCGGTTCCGGCGGAGGATCTCTTCAAT (SEQ ID NO: 292). In some embodiments, nucleic acids encoding SEQ ID NO: 289 may comprise SEQ ID NO: 292, or a nucleic acid sequence that is at least 90%, at least 91%, at least 92%, at least 93%, at least 94%, at least 95%, at least 96%, at least 97%, at least 98%, or at least 99% identical to SEQ ID NO: 292.

In some embodiments, the protease cleavage site comprises the amino acid sequence of ITQGLAVSTISSFF (SEQ ID NO: 198), which is a cleavage site that is native to CD16 and is cleavable by ADAM17. In certain embodiments, SEQ ID NO: 198 is comprised within a linker. In certain embodiments, the linker comprises the amino acid sequence of SGGGGSGGGGSGITQGLAVSTISSFFGGGSGGGGSGGGSLQ (SEQ ID NO: 290). An exemplary nucleic acid sequence encoding SEQ ID NO: 290 is AGCGGCGGAGGTGGTAGCGGAGGCGGAGGATCTGGAATTACACAGGGACTCGCCG TGTCTACAATCTCCAGCTTCTTTGGTGGCGGTAGTGGCGGCGGTGGCAGTGGCGGTG GATCTCTTCAA (SEQ ID NO: 291). In some embodiments, nucleic acids encoding SEQ ID NO: 290 may comprise SEQ ID NO: 291, or a nucleic acid sequence that is at least 90%, at least 91%, at least 92%, at least 93%, at least 94%, at least 95%, at least 96%, at least 97%, at least 98%, or at least 99% identical to SEQ ID NO: 291.

The protease cleavage site can be C-terminal of the secretable effector molecule. The protease cleavage site can be N-terminal of the secretable effector molecule. In general, for all membrane-cleavable chimeric proteins described herein, the protease cleavage site is either: (1) C-terminal of the secretable effector molecule and N-terminal of the cell membrane tethering domain (in other words, the protease cleavage site is in between the secretable effector molecule and the cell membrane tethering domain); or (2)N-terminal of the secretable effector molecule and C-terminal of the cell membrane tethering domain (also between the secretable effector molecule and the cell membrane tethering domain with domain orientation inverted). The protease cleavage site can be connected to the secretable effector molecule by a polypeptide linker, i.e., a polypeptide sequence not generally considered to be part of the effector molecule or protease cleavage site. The protease cleavage site can be connected to the cell membrane tethering domain by a polypeptide linker, i.e., a polypeptide sequence not generally considered to be part of the cell membrane tethering domain or protease cleavage site. A polypeptide linker can be any amino acid sequence that connects a first polypeptide sequence and a second polypeptide sequence. A polypeptide linker can be a flexible linker (e.g., a Gly-Ser-Gly sequence). Examples of polypeptide linkers include, but are not limited to, GSG linkers (e.g., [GS]₄GG [SEQ ID NO: 347]), A(EAAAK)₃A (SEQ ID NO: 348), and Whitlow linkers (e.g., a “KEGS (SEQ ID NO: 446)” linker such as the amino acid sequence KESGSVSSEQLAQFRSLD (SEQ ID NO: 349), an eGK linker such as the amino acid sequence EGKSSGSGSESKST (SEQ ID NO: 350), an LR1 linker such as the amino acid sequence SGGGGSGGGGSGGGGSGGGGSGGGSLQ (SEQ ID NO: 215), the amino acid sequence GSTSGSGKPGSGEGSTKG (SEQ ID NO: 395), and linkers described in more detail in Issued U.S. Pat. No. 5,990,275 herein incorporated by reference). Additional exemplary polypeptide linkers include SGGGGSGGGGSG (SEQ ID NO: 194), TTTPAPRPPTPAPTIASQPLSLRPEACRPAAGGAVHTRGLDFACD (SEQ ID NO: 196), and GGGSGGGGSGGGSLQ (SEQ ID NO: 197). Other polypeptide linkers may be selected based on desired properties (e.g., length, flexibility, amino acid composition, etc.) and are known to those skilled in the art. An exemplary nucleic acid sequence encoding SEQ ID NO: 196 is ACCACCACACCAGCTCCTCGGCCACCAACTCCAGCTCCAACAATTGCCAGCCAGCC TCTGTCTCTGAGGCCCGAAGCTTGTAGACCTGCTGCAGGCGGAGCCGTGCATACAA GAGGACTGGATTTCGCCTGCGAC (SEQ ID NO: 337). In certain embodiments, a nucleic acid encoding SEQ ID NO: 196 comprises a sequence that is at least 90%, at least 91%, at least 92%, at least 93%, at least 94%, at least 95%, at least 96%, at least 97%, at least 98%, or at least 99% identical to SEQ ID NO: 337. Yet other polypeptide linkers include GGSGSGGSGS (SEQ ID NO: 396) and SAGSGSGASGSG (SEQ ID NO: 397).

In the Membrane-Cleavable system, following expression and localization of the chimeric protein into the cell membrane, the protease cleavage site directs cleavage of the chimeric protein such that the effector molecule is released (“secreted”) into the extracellular space of a cell.

In general, a protease that cleaves the protease cleavage site is a protease specific for that specific protease cleavage site. For example, in the case of a disintegrin and metalloproteinase (“ADAM”) family protease, the protease that cleaves a specific ADAM protease cleavage site is generally limited to the ADAM protease(s) that specifically recognize the specific ADAM protease cleavage site motif. A protease cleavage site can be selected and/or engineered such that cleavage by undesired proteases is reduced or eliminated. Proteases can be membrane-bound or membrane-associated. Proteases can be secreted, e.g., secreted in a specific cellular environment, such as a tumor microenvironment (“TME”).

A protease that cleaves the protease cleavage site of the chimeric protein can be expressed in the same cell that expresses the chimeric protein. A protease that cleaves the protease cleavage site of the chimeric protein can be endogenous to a cell expressing the chimeric protein. In other words, a cell engineered to express the chimeric protein can endogenously express the protease specific for the protease cleavage site present in the chimeric protein. Endogenous expression of the protease refers to both expression under generally homeostatic conditions (e.g., a cell generally considered to be healthy), and also to differential expression under non-homeostatic conditions (e.g., upregulated expression in a tumor cell). The protease cleavage site can be selected based on the known proteases endogenously expressed by a desired cell population. In such cases, in general, the cleavage of the protease cleavage site (and thus release/secretion of a payload) can be restricted to only those cells of interest due to the cell-restricted protease needing to come in contact with the protease cleavage site of chimeric protein expressed in the same cell. For example, and without wishing to be bound by theory, ADAM17 is believed to be restricted in its endogenous expression to NK cell and T cells. Thus, selection of an ADAM17-specific protease cleavage site may restrict the cleavage of the protease cleavage site to NK cell and T cells co-expressing the chimeric protein. In other examples, a protease cleavage site can be selected for a specific tumor-associated protease known to be expressed in a particular tumor population of interest (e.g., in a specific tumor cell engineered to express the chimeric protein). Protease and/or expression databases can be used to select an appropriate protease cleavage site, such as selecting a protease cleavage site cleaved by a tumor-associated proteases through consulting Oncomine (www.oncomine.org), the European Bioinformatic Institute (www.ebi.ac.uk) in particular (www.ebi.ac.uk/gxa), PMAP (www.proteolysis.org), ExPASy Peptide Cutter (ca.expasy.org/tools/peptide cutter) and PMAP.Cut DB (cutdb.bumham.org), each of which is incorporated by reference for all purposes.

A protease that cleaves the protease cleavage site of the chimeric protein can be heterologous to a cell expressing the chimeric protein. For example, a cell engineered to express the chimeric protein can also be engineered to express a protease not generally expressed by the cell that is specific for the protease cleavage site present in the chimeric protein. A cell engineered to express both the chimeric protein and the protease can be engineered to express each from separate engineered nucleic acids or from a multicistronic systems (multicistronic and multi-promoter systems are described in greater detail in the Section herein titled “Multicistronic and Multiple Promoter Systems”). Heterologous proteases and their corresponding protease cleavage site can be selected as described above with reference to endogenous proteases.

A protease that cleaves the protease cleavage site of the chimeric protein can be expressed on a separate distinct cell than the cell that expresses the chimeric protein. For example, the protease can be generally expressed in a specific cellular environment, such as a tumor microenvironment. In such cases, in general, the cleavage of the protease cleavage site can be restricted to only those cellular environments of interest (e.g., a tumor microenvironment) due to the environment-restricted protease needing to come in contact with the protease cleavage site. In embodiments having membrane-cleavable chimeric proteins, in general, the secretion of the effector molecule can be restricted to only those cellular environments of interest (e.g., a tumor microenvironment) due to the environment-restricted protease needing to come in contact with the protease cleavage site. A protease that cleaves the protease cleavage site of the chimeric protein can be endogenous to the separate distinct cell. A protease that cleaves the protease cleavage site of the chimeric protein can be heterologous to the separate distinct cell. For example, the separate distinct cell can be engineered to express a protease not generally expressed by the separate distinct cell.

Proteases include, but are not limited to, a Type 1 transmembrane protease, a Type II transmembrane protease, a GPI anchored protease, an ADAM8 protease, an ADAM9 protease, an ADAM10 protease, an ADAM12 protease, an ADAM15 protease, an ADAM17 protease, an ADAM19 protease, an ADAM20 protease, an ADAM21 protease, an ADAM28 protease, an ADAM3(protease, an ADAM33 protease, a BACE1 protease, a BACE2 protease, a SIP protease, an MT1-MMP protease, an MT3-MMP protease, an MT5-MMP protease, a furin protease, a PCSK7 protease, a matriptase protease, a matrptase-2 protease, and an MMP9 protease. A protease can be an NS3 protease. A protease can be an ADAM17 protease.

Proteases can be tumor associated proteases, such as, a cathepsin, a cystene protease, an aspartyl protease, a serine protease, or a metalloprotease. Specific examples of tumor associated proteases include Cathepsin B, Cathepsin L, Cathepsin 5, Cathepsin D, Cathepsin F, Cathepsin A, Cathepsin G, Thrombin, Plasmin, Urokinase, Tissue Plasminogen Activator, Metalloproteinase 1 (MMP1), MMP2, MMP3, MMP4, MMP7, MMP8, MMP9, MMP10, MMP11, MMP12, MMP13, MMP14, MMP15, MMP16, MMP17, MMP20, MMP21, MMP23, MMP24, MMP25, MMP26, MMP28, ADAM, ADAMTS, CD10 (CALLA), or prostate specific antigen. Proteases can also include, but are not limited to, proteases listed in Table 4B below. Exemplary cognate protease cleavage sites for certain proteases are also listed in Table 4B.

TABLE 4B
Exemplary Proteases with Cognate Cleavage Sites and Inhibitors

| Protease | | |
|---|---|---|
| (UniProt Accession No.) | Cognate cleavage site | Protease inhibitors |
| HCV NS4A/4B | DEMEECSQHL | Simeprevir, Danoprevir, |
| | (SEQ ID NO: 142) | Asunaprevir, Ciluprevir, |
| | EDVVPCSMG | Boceprevir, Sovaprevir, |
| | (SEQ ID NO: 143) | Paritaprevir, Telaprevir, |
| | | Grazoprevir |
| HCV NS5A/5B | DEMEECSQHL | Simeprevir, Danoprevir, |
| | (SEQ ID NO: 142) | Asunaprevir, Ciluprevir, |
| | EDVVPCSMG | Boceprevir, Sovaprevir, |
| | (SEQ ID NO: 143) | Paritaprevir, Telaprevir, |
| | | Grazoprevir |
| HCV NS3 | DEMEECSQHL | Simeprevir, Danoprevir, |
| | (SEQ ID NO: 142) | Asunaprevir, Ciluprevir, |
| | EDVVPCSMG | Boceprevir, Sovaprevir, |
| | (SEQ ID NO: 143) | Paritaprevir, Telaprevir, |
| | | Grazoprevir |
| HCV NS2-3 | DEMEECSQHL | Simeprevir, Danoprevir, |
| | (SEQ ID NO: 142) | Asunaprevir, Ciluprevir, |
| | EDVVPCSMG | Boceprevir, Sovaprevir, |
| | (SEQ ID NO: 143) | Paritaprevir, Telaprevir, |
| | | Grazoprevir |
| HIV-1 protease | | Amprenavir, Atazanavir, |
| (SEQ ID NO: 144) | | Darunavir, Fosamprenavir, |
| | | Indinavir, Lopinavir, |
| | | Nelfinavir, Ritonavir, |
| | | Saquinavir, Tipranavir |
| Signal peptidase (P67812, | preference of eukaryotic signal | |
| P15367, P00804, P0803) | peptidase for cleavage after | |
| | residue 20 (Xaa^{20↓}) of | |
| | pre(Apro)apoA-II: Ala, Cys > Gly | |
| | > Ser, Thr > Pro > Asn, Val, Ile, | |
| | Leu, Tyr, His, Arg, Asp. | |
| proprotein convertases | (R/K)-X-(hydrophobic)-X↓, where | |
| cleaving at hydrophobic | X is any amino acid | |
| residues (e.g., Leu, Phe, | | |
| Val, or Met) (Q16549, | | |
| Q8NBP7, Q92824, | | |
| P29120, Q6UW60, | | |
| P29122, Q9QXV0) | | |
| proprotein convertases | (K/R)-(X)n-(K/R)↓, where n is 0, | |
| cleaving at small amino | 2, 4 or 6 and X is any amino acid | |
| acid residues such as Ala | | |
| or Thr (Q16549, | | |
| Q8NBP7, Q92824, | | |
| P29120, Q6UW60, | | |
| P29122) | | |
| proopiomelanocortin | Cleavage at paired basic residues | |
| converting enzyme (PCE) | in certain prohormones, either | |
| (Q9UO77615, 0776133) | between them, or on the carboxyl | |
| | side | |
| chromaffin granule | tends to cleave dipeptide bonds | |
| aspartic protease (CGAP) | that have hydrophobic residues as | |
| | well as a beta-methylene group | |
| prohormone thiol protease | | |
| (cathepsin L1) (P07154, | | |
| P07711, P06797, P25975, | | |
| Q28944) | | |
| carboxypeptidases (e.g., | cleaves a peptide bond at the | |
| carboxypeptidase E/H, | carboxy-terminal (C-terminal) end | |
| carboxypeptidase D and | of a protein or peptide | |
| carboxypeptidase Z) | | |
| (Q9M099, P15169, | | |
| Q04609, P08819, P08818, | | |
| 077564, P70627, | | |
| 035409, P07519, | | |
| Q8VZU3, P22792, | | |
| P15087, P16870, | | |
| Q9JHH6, Q96IY4, | | |
| Q7L8A9) | | |
| aminopeptidases (e.g., | cleaves a peptide bond at the | |
| arginine aminopeptidase, | amino-terminal (N-terminal) end | |
| lysine aminopeptidase, | of a protein or peptide | |
| aminopeptidase B) | | |
| prolyl endopeptidase | Hydrolysis of Pro-\|-Xaa >> Ala-\|- | |
| (Q12884, P48147, | Xaa in oligopeptides. | |
| P97321, Q4J6C6) | Release of an N-terminal | |
| | dipeptide, Xaa-Yaa-\|-Zaa-, from a | |
| | polypeptide, preferentially when | |
| | Yaa is Pro, provided Zaa is neither | |
| | Pro nor hydroxyproline | |
| aminopeptidase N | Release of an N-terminal amino | |
| (P97449, P15144, | acid, Xaa-\|-Yaa- from a peptide, | |
| P15145, P15684) | amide or arylamide. Xaa is | |
| | preferably Ala, but may be most | |
| | amino acids including Pro (slow | |
| | action). When a terminal | |
| | hydrophobic residue is followed | |
| | by a prolyl residue, the two may | |
| | be released as an intact Xaa-Pro | |
| | dipeptide | |
| insulin degrading enzyme | Degradation of insulin, glucagon | |
| (P14735, P35559, | and other polypeptides. No action | |
| Q9JHR7, P22817, | on proteins. | |
| Q24K02) | Cleaves multiple short | |
| | polypeptides that vary | |
| | considerably in sequence | |
| Calpain (O08529, | No specific amino acid sequence | |
| P17655, Q07009, | is uniquely recognized by | |
| Q27971, P20807, P07384, | calpains. Amongst protein | |
| 035350, 014815, | substrates, tertiary structure | |
| P04632, Q9Y6Q1, | elements rather than primary | |
| 015484, Q9HC96, | amino acid sequences appear to be | |
| A6NHCO, Q9UMQ6) | responsible for directing cleavage | |
| | to a specific substrate. Amongst | |
| | peptide and small-molecule | |
| | substrates, the most consistently | |
| | reported specificity is for small, | |
| | hydrophobic amino acids (e.g., | |
| | leucine, valine and isoleucine) at | |
| | the P2 position, and large | |
| | hydrophobic amino acids (e.g., | |
| | phenylalanine and tyrosine) at the | |
| | P1 position. One fluorogenic | |
| | calpain substrate is (EDANS)-Glu- | |
| | Pro-Leu-Phe═Ala-Glu-Arg-Lys- | |
| | (DABCYL) (SEQ ID NO: 145), | |
| | (EDANSEPLFAERKDABCYL | |
| | (SEQ ID NO: 145)) with cleavage | |
| | occurring at the Phe═Ala bond. | |
| caspase 1 (P29466, | Strict requirement for an Asp | |
| P29452) | residue at position P1 and has a | |
| | preferred cleavage sequence of | |
| | Tyr-Val-Ala-Asp- (SEQ ID NO: | |
| | 146)\|- (YVAD; SEQ ID NO: 146). | |
| caspase 2 (P42575, | Strict requirement for an Asp | |
| P29594) | residue at P1, with 316-asp being | |
| | essential for proteolytic activity | |
| | and has a preferred cleavage | |
| | sequence of Val-Asp-Val-Ala- | |
| | Asp- (SEQ ID NO: 147\|- | |
| | (VDVAD; SEQ ID NO: 147). | |
| caspase 3 (P42574, | Strict requirement for an Asp | |
| P70677) | residue at positions P1 and P4. It | |
| | has a preferred cleavage sequence | |
| | of Asp-Xaa-Xaa-Asp-\|- with a | |
| | hydrophobic amino-acid residue at | |
| | P2 and a hydrophilic amino-acid | |
| | residue at P3, although Val or Ala | |
| | are also accepted at this position. | |
| caspase 4 (P70343, | Strict requirement for Asp at the | |
| P49662) | P1 position. It has a preferred | |
| | cleavage sequence of Tyr-Val- | |
| | Ala-Asp- (SEQ ID NO: 146)\|- | |
| | (YVAD; SEQ ID NO: 146) but | |
| | also cleaves at Asp-Glu-Val-Asp- | |
| | (SEQ ID NO: 148)\|-(DEVD; SEQ | |
| | ID NO: 148). | |
| caspase 5 (P51878) | Strict requirement for Asp at the | |
| | P1 position. It has a preferred | |
| | cleavage sequence of Tyr-Val- | |
| | Ala-Asp- (SEQ ID NO: 146)\|- | |
| | (YVAD; SEQ ID NO: 146) but | |
| | also cleaves at Asp-Glu-Val-Asp- | |
| | (SEQ ID NO: 148)\|--\|-(DEVD; | |
| | SEQ ID NO: 148). | |
| caspase 6 (P55212) | Strict requirement for Asp at | |
| | position P1 and has a preferred | |
| | cleavage sequence of Val-Glu- | |
| | His-Asp- (SEQ ID NO: 149)\|- | |
| | (VEHD; SEQ ID NO: 149). | |
| caspase 7 (P97864, | Strict requirement for an Asp | |
| P55210) | residue at position P1 and has a | |
| | preferred cleavage sequence of | |
| | Asp-Glu-Val-Asp- (SEQ ID NO: | |
| | 148)\|- (DEVD; SEQ ID NO: 148). | |
| caspase 8 (Q8IRY7, | Strict requirement for Asp at | |
| 089110, Q14790) | position P1 and has a preferred | |
| | cleavage sequence of | |
| | (Leu/Asp/Val)-Glu-Thr-Asp-\|- | |
| | (Gly/Ser/Ala). | |
| caspase 9 (P55211, | Strict requirement for an Asp | |
| Q8C3Q9, Q5IS54) | residue at position Pl and with a | |
| | marked preference for His at | |
| | position P2. It has a preferred | |
| | cleavage sequence of Leu-Gly- | |
| | His-Asp-\|-Xaa(SEQ ID NO:445) | |
| | (LGHD; SEQ ID NO: 150). | |
| caspase 10 (Q92851) | Strict requirement for Asp at | |
| | position Pl and has a preferred | |
| | cleavage sequence of Leu-Gln- | |
| | Thr-Asp-\|-Gly (SEQ ID NO: 151) | |
| | (LQTDG; SEQ ID NO: 151). | |
| puromycin sensitive | Release of an N-terminal amino | |
| aminopeptidase (P55786, | acid, preferentially alanine, from a | |
| Q11011) | wide range of peptides, amides | |
| | and arylamides. | |
| angiotensin converting | Release of a C-terminal dipeptide, | Benazepril (Lotensin), |
| enzyme (ACE) (P12821, | oligopeptide-\|-Xaa-Yaa, when Xaa | Captopril, Enalapril |
| P09470, Q9BYF1) | is not Pro, and Yaa is neither Asp | (Vasotec), Fosinopril, |
| SEQ ID NO: 156 | nor Glu. | Lisinopril (Prinivil, |
| | | Zestril), Moexipril, |
| | | Perindopril (Aceon), |
| | | Quinapril (Accupril), |
| | | Ramipril (Altace), |
| | | Trandolapril (Mavik), |
| | | Zofenopril |
| pyroglutamyl peptidase II | Release of the N-terminal | |
| (Q9NXJ5) | pyroglutamyl group from pGlu-- | |
| | His-Xaa tripeptides and pGlu-- | |
| | His-Xaa-Gly tetrapeptides | |
| dipeptidyl peptidase IV | Release of an N-terminal | |
| (P27487, P14740, | dipeptide, Xaa-Yaa-\|-Zaa-, from a | |
| P28843) | polypeptide, preferentially when | |
| | Yaa is Pro, provided Zaa is neither | |
| | Pro nor hydroxyproline | |
| N-arginine dibasic | Hydrolysis of polypeptides, | |
| convertase (043847, | preferably at -Xaa-\|-Arg-Lys-, and | |
| Q8BHG1) | less commonly at -Arg-\|-Arg-Xaa-, | |
| | in which Xaa is not Arg or Lys | |
| endopeptidase 24.15 | Preferential cleavage of bonds | |
| (thimet oligopeptidase) | with hydrophobic residues at P1, | |
| (P52888, P24155) | P2 and P3′ and a small residue at | |
| | P1′ in substrates of 5 to 15 | |
| | residues | |
| endopeptidase 24.16 | Preferential cleavage in | |
| (neurolysin) (Q9BYT8, | neurotensin: 10-Pro-\|-Tyr-11 | |
| Q91YP2) | | |
| amyloid precursor protein | Endopeptidase of broad | |
| secretase alpha (P05067, | specificity. | |
| P12023, Q9Y5Z0, | | |
| P56817) | | |
| amyloid precursor protein | Broad endopeptidase specificity. | |
| secretase beta (P05067, | Cleaves Glu-Val-Asn-Leu-\|-Asp- | |
| P12023, Q9Y5Z0, | Ala-Glu-Phe (SEQ ID NO: 152) | |
| P56817) | (EVNLDAEF; SEQ ID NO: 152) | |
| | in the Swedish variant of | |
| | Alzheimer's amyloid precursor | |
| | protein | |
| amyloid precursor protein | intramembrane cleavage of | |
| secretase gamma | integral membrane proteins | |
| (P05067, P12023, | | |
| Q9Y5Z0, P56817) | | |
| MMP 1 (P03956, | Cleavage of the triple helix of | SB-3CT |
| Q9EPL5uy) | collagen at about three-quarters of | p-OH SB-3CT |
| | the length of the molecule from | O-phosphate SB-3CT |
| | the N-terminus, at 775-Gly-\|-Ile- | RXP470.1 |
| | 776 in the alpha-1 (I) chain. | |
| | Cleaves synthetic substrates and | |
| | alpha-macroglobulins at bonds | |
| | where P1′ is a hydrophobic | |
| | residue. | |
| MMP 2 (P08253, P33434) | Cleavage of gelatin type I and | SB-3CT |
| | collagen types IV, V, VII, X. | p-OH SB-3CT |
| | Cleaves the collagen-like sequence | O-phosphate SB-3CT |
| | Pro-Gln-Gly-\|-Ile-Ala-Gly-Gln | RXP470.1 |
| | (SEQ ID NO: 153) (PQGIAGQ; | |
| | SEQ ID NO: 153). | |
| MMP 3 (P08254, P28862) | Preferential cleavage where P1′, | SB-3CT |
| | P2′ and P3′ are hydrophobic | p-OH SB-3CT |
| | residues. | O-phosphate SB-3CT |
| | | RXP470.1 |
| MMP 7 (P09237, | Cleavage of 14-Ala-\|-Leu-15 and | SB-3CT |
| Q10738) | 16-Tyr-\|-Leu-17 in B chain of | p-OH SB-3CT |
| | insulin. No action on collagen | O-phosphate SB-3CT |
| | types I, II, IV, V. Cleaves gelatin | RXP470.1 |
| | chain alpha-2(I) > alpha-1(I). | |
| MMP 8 (P22894, | Can degrade fibrillar type I, II, and | SB-3CT |
| O70138) | III collagens. | p-OH SB-3CT |
| | Cleavage of interstitial collagens | O-phosphate SB-3CT |
| | in the triple helical domain. Unlike | RXP470.1 |
| | EC 3.4.24.7, this enzyme cleaves | |
| | type III collagen more slowly than | |
| | type I. | |
| MMP 9 (P14780, P41245) | Cleavage of gelatin types I and V | SB-3CT |
| | and collagen types IV and V. | p-OH SB-3CT |
| | Cleaves KiSS1 at a Gly-\|-Leu | O-phosphate SB-3CT |
| | bond. | RXP470.1 |
| | Cleaves type IV and type V | |
| | collagen into large C-terminal | |
| | three quarter fragments and | |
| | shorter N-terminal one quarter | |
| | fragments. Degrades fibronectin | |
| | but not laminin or Pz-peptide. | |
| MMP 10 (P09238, | Can degrade fibronectin, gelatins | SB-3CT |
| 055123) | of type I, III, IV, and V; weakly | p-OH SB-3CT |
| | collagens III, IV, and V. | O-phosphate SB-3CT |
| | | RXP470.1 |
| MMP 11 (P24347, | A(A/Q)(N/A)↓(L/Y)(T/V/M/R)(R/ | SB-3CT |
| Q02853) | K) | p-OH SB-3CT |
| | G(G/A)E↓LR (SEQ ID NO: 344) | O-phosphate SB-3CT |
| | ↓ denotes the cleavage site | RXP470.1 |
| MMP 12 (P39900, | Hydrolysis of soluble and | SB-3CT |
| P34960) | insoluble elastin. Specific | p-OH SB-3CT |
| | cleavages are also produced at 14- | O-phosphate SB-3CT |
| | Ala-\|-Leu-15 and 16-Tyr-\|-Leu-17 | RXP470.1 |
| | in the B chain of insulin | |
| | Has significant elastolytic activity. | |
| | Can accept large and small amino | |
| | acids at the P1′ site, but has a | |
| | preference for leucine. Aromatic | |
| | or hydrophobic residues are | |
| | preferred at the Pl site, with small | |
| | hydrophobic residues (preferably | |
| | alanine) occupying P3 | |
| MMP 13 (P45452, | Cleaves triple helical collagens, | SB-3CT |
| P33435) | including type I, type II and type | p-OH SB-3CT |
| | III collagen, but has the highest | O-phosphate SB-3CT |
| | activity with soluble type II | RXP470.1 |
| | collagen. Can also degrade | |
| | collagen type IV, type XIV and | |
| | type X | |
| MMP 14 (P50281, | Activates progelatinase A by | SB-3CT |
| P53690) | cleavage of the propeptide at 37- | p-OH SB-3CT |
| | Asn-\|-Leu-38. Other bonds | O-phosphate SB-3CT |
| | hydrolyzed include 35-Gly-\|-Ile-36 | RXP470.1 |
| | in the propeptide of collagenase 3, | |
| | and 341-Asn-\|-Phe-342, 441-Asp- | |
| | \|-Leu-442 and 354-Gln-\|-Thr-355 | |
| | in the aggrecan interglobular | |
| | domain. | |
| urokinase plasminogen | Specific cleavage of Arg-\|-Val | Plasminogen activator |
| activator (uPA) (P00749, | bond in plasminogen to form | inhibitors (PAI) |
| P06869) | plasmin. | |
| tissue plasminogen | Specific cleavage of Arg-\|-Val | Plasminogen activator |
| activator (tPA) (P00750, | bond in plasminogen to form | inhibitors (PAI) |
| P11214) | plasmin. | |
| tissue plasminogen | Specific cleavage of Arg-\|-Val | Plasminogen activator |
| activator (tPA) (P00750, | bond in plasminogen to form | inhibitors (PAI) |
| P11214) | plasmin. | |
| Plasmin (P00747, | Preferential cleavage: Lys-\|-Xaa > | α-2-antiplasmin (AP) |
| P20918) | Arg-\|-Xaa, higher selectivity than | |
| | trypsin. Converts fibrin into | |
| | soluble products. | |
| Thrombin (P00734, | Cleaves bonds after Arg and Lys | |
| P19221) | Converts fibrinogen to fibrin and | |
| | activates factors V, VII, VIII, XIII, | |
| | and, in complex with | |
| | thrombomodulin, protein C. | |
| BMP-1 (procollagen C- | Cleavage of the C-terminal | |
| peptidase) (P13497, | propeptide at Ala-\|-Asp in type I | |
| P98063) | and II procollagens and at Arg-\|- | |
| | Asp in type III. | |
| ADAM (Q9POK1, | | SB-3CT |
| Q9UKQ2, Q9JLN6, | | p-OH SB-3CT |
| 014672, Q13444, | | O-phosphate SB-3CT |
| P78536, Q13443, | | RXP470.1 |
| 043184, P78325, | | |
| Q9UKF5, Q9BZ11, | | |
| Q9H2U9, Q99965, | | |
| 075077, Q9H013, | | |
| 043506) | | |
| granzyme A (P12544, | Preferential cleavage: -Arg-\|-Xaa-, | |
| P11032) | -Lys-\|-Xaa- >> -Phe-\|-Xaa- in | |
| | small molecule substrates. | |
| granzyme B (P10144, | Preference for bulky and aromatic | |
| P04187) | residues at the P1 position and | |
| | acidic residues at the P3′ and P4′ | |
| | sites. | |
| granzyme M (P51124, | Cleaves peptide substrates after | |
| Q03238) | methionine, leucine, and | |
| | norleucine. | |
| tobacco Etch virus (TEV) | E-Xaa-Xaa-Y -Xaa-Q-(G/S), with | |
| protease (P04517, | cleavage occurring between Q and | |
| POCK09) | G/S. The most common sequence | |
| | is ENLYFQS (SEQ ID NO: 154) | |
| chymotrypsin-like serine | | -Thermobifida fusca |
| protease (P08217, | | Thermopin |
| Q9UNI1, Q91X79, | | -Pyrobaculum aerophilum |
| P08861, P09093, P08218) | | Aeropin |
| | | -Thermococcus |
| | | kodakaraensis Tk-serpin |
| | | -Alteromonas sp. |
| | | Marinostatin |
| | | -Streptomyces misionensis |
| | | SMTI |
| | | -Streptomyces sp. |
| | | chymostatin |
| alphavirus proteases | | |
| (P08411, P03317, | | |
| P13886, Q8JUX6, | | |
| Q86924, Q4QXJ8, | | |
| Q8QL53, P27282, | | |
| Q5XXP4) | | |
| chymotrypsin-like | | -Thermobifida fusca |
| cysteine proteases | | Thermopin |
| (Q86TL0, Q14790, | | -Pyrobaculum aerophilum |
| Q99538, 015553) | | Aeropin |
| | | -Thermococcus |
| | | kodakaraensis Tk-serpin |
| | | -Alteromonas sp. |
| | | Marinostatin |
| | | -Streptomyces misionensis |
| | | SMTI |
| | | -Streptomyces sp. |
| | | chymostatin |
| papain-like cysteine | | |
| proteases (P25774, | | |
| P53634, Q96K76) | | |
| picornavirus leader | | |
| proteases (P03305, | | |
| P03311, P13899) | | |
| HIV proteases (P04585, | | |
| P03367, P04584, P03369, | | |
| P12497, P03366, P04587) | | |
| Herpesvirus proteases | | |
| (P10220, Q2HRB6, | | |
| 040922, Q69527) | | |
| adenovirus proteases | | |
| (P03252, P24937, | | |
| Q83906, P68985, P09569, | | |
| P11825, P10381) | | |
| Streptomyces griseus | | |
| protease A (SGPA) | | |
| (P00776) | | |
| Streptomyces griseus | | |
| protease B (SGPB) | | |
| (P00777) | | |
| alpha-lytic protease | | |
| (P85142, P00778) | | |
| serine proteases (P48740, | | |
| P98064, Q9UL52, | | |
| P05981, 060235) | | |
| cysteine proteases | | |
| (Q86TL0, Q14790, | | |
| Q8WYN0, Q96DT6, | | |
| P55211) | | |
| aspartic proteases | | |
| (Q9Y5Z0, P56817, | | |
| Q00663, Q53RT3, | | |
| POCY27) | | |
| threonine proteases | | |
| (Q9UI38, Q16512, | | |
| Q9H6P5, Q8IWU2) | | |
| Mast cell (MC) chymase | Abz-HPFHL-Lys(Dnp)-NH2(SEQ | BAY 1142524 |
| (CMA1) (NM 001836) | ID NO: 155) | SUN13834 |
| Rat mast cell protease-1, | Abz-HPFHL-Lys(Dnp)-NH2(SEQ | TY-51469 |
| -2, -3, -4, -5 (NM_017145, | ID NO: 155) | |
| NM 172044, | | |
| NM_001170466, | | |
| NM_019321, | | |
| NM_013092) | | |
| Rat vascular chymase | Abz-HPFHL-Lys(Dnp)-NH2(SEQ | |
| (RVCH) (070500) | ID NO: 155) | |
| DENV NS3pro | A strong preference for basic | Anthraquinone |
| (NS2B/NS3) | amino acid residues (Arg/Lys) at | BP13944 |
| SEQ ID NOs: 157, 158, | the P1 positions was observed, | ZINC04321905 |
| 159, 160 | whereas the preferences for the | MB21 |
| | P2- 4 sites were in the order of | Policresulen |
| | Arg > Thr > Gln/Asn/Lys for P2, | SK-12 |
| | Lys > Arg > Asn for P3, and Nle > | NSC135618 |
| | Leu > Lys > Xaa for P4. The | Biliverdin |
| | prime site substrate specificity was | |
| | for small and polar amino acids in | |
| | P1 and P3. | |

A protease can be any of the following human proteases (MEROPS peptidase database number provided in parentheses; Rawlings N. D., Morton F. R., Kok, C. Y., Kong, J. & Barrett A. J. (2008) MEROPS: the peptidase database. Nucleic Acids Res. 36 Database issue, D320-325; herein incorporated by reference for all purposes): pepsin A (MER000885), gastricsin (MER000894), memapsin-2 (MER005870), renin (MER000917), cathepsin D (MER000911), cathepsin F (MER000944), memapsin-1 (MER005534), napsin A (MER004981), Memname-AA034 peptidase (MER014038), pepsin A4 (MER037290), pepsin AS (Homo sapiens) (MER037291), hCG1733572 (Homo sapiens)-type putative peptidase (MER107386), napsin B pseudogene (MER004982), CYMP g.p. (Homo sapiens) (MER002929), subfamily AIA unassigned peptidases (MER181559), mouse mammary tumor virus retropepsin (MER048030), rabbit endogenous retrovirus endopeptidase (MER043650), S71-related human endogenous retropepsin (MER001812), RTVL-H-type putative peptidase (MER047117), RTVL-H-type putative peptidase (MER047133), RTVL-H-type putative peptidase (MER047160), RTVL-H-type putative peptidase (MER047206), RTVL-H-type putative peptidase (MER047253), RTVL-H-type putative peptidase (MER047260), RTVL-H-type putative peptidase (MER047291), RTVL-H-type putative peptidase (MER047418), RTVL-H-type putative peptidase (MER047440), RTVL-H-type putative peptidase (MER047479), RTVL-H-type putative peptidase (MER047559), RTVL-H-type putative peptidase (MER047583), RTVL-H-type putative peptidase (MER015446), human endogenous retrovirus retropepsin homologue 1 (MER015479), human endogenous retrovirus retropepsin homologue 2 (MER015481), endogenous retrovirus retropepsin pseudogene 1 (Homo sapiens chromosome 14) (MER029977), endogenous retrovirus retropepsin pseudogene 2 (Homo sapiens chromosome 8) (MER029665), endogenous retrovirus retropepsin pseudogene 3 (Homo sapiens chromosome 17) (MER002660), endogenous retrovirus retropepsin pseudogene 3 (Homo sapiens chromosome 17) (MER030286), endogenous retrovirus retropepsin pseudogene 3 (Homo sapiens chromosome 17) (MER047144), endogenous retrovirus retropepsin pseudogene 5 (Homo sapiens chromosome 12) (MER029664), endogenous retrovirus retropepsin pseudogene 6 (Homo sapiens chromosome 7) (MER002094), endogenous retrovirus retropepsin pseudogene 7 (Homo sapiens chromosome 6) (MER029776), endogenous retrovirus retropepsin pseudogene 8 (Homo sapiens chromosome Y) (MER030291), endogenous retrovirus retropepsin pseudogene 9 (Homo sapiens chromosome 19) (MER029680), endogenous retrovirus retropepsin pseudogene 10 (Homo sapiens chromosome 12) (MER002848), endogenous retrovirus retropepsin pseudogene 11 (Homo sapiens chromosome 17) (MER004378), endogenous retrovirus retropepsin pseudogene 12 (Homo sapiens chromosome 11) (MER003344), endogenous retrovirus retropepsin pseudogene 13 (Homo sapiens chromosome 2 and similar) (MER029779), endogenous retrovirus retropepsin pseudogene 14 (Homo sapiens chromosome 2) (MER029778), endogenous retrovirus retropepsin pseudogene 15 (Homo sapiens chromosome 4) (MER047158), endogenous retrovirus retropepsin pseudogene 15 (Homo sapiens chromosome 4) (MER047332), endogenous retrovirus retropepsin pseudogene 15 (Homo sapiens chromosome 4) (MER003182), endogenous retrovirus retropepsin pseudogene 16 (MER047165), endogenous retrovirus retropepsin pseudogene 16 (MER047178), endogenous retrovirus retropepsin pseudogene 16 (MER047200), endogenous retrovirus retropepsin pseudogene 16 (MER047315), endogenous retrovirus retropepsin pseudogene 16 (MER047405), endogenous retrovirus retropepsin pseudogene 16 (MER030292), endogenous retrovirus retropepsin pseudogene 17 (Homo sapiens chromosome 8) (MER005305), endogenous retrovirus retropepsin pseudogene 18 (Homo sapiens chromosome 4) (MER030288), endogenous retrovirus retropepsin pseudogene 19 (Homo sapiens chromosome 16) (MER001740), endogenous retrovirus retropepsin pseudogene 21 (Homo sapiens) (MER047222), endogenous retrovirus retropepsin pseudogene 21 (Homo sapiens) (MER047454), endogenous retrovirus retropepsin pseudogene 21 (Homo sapiens) (MER047477), endogenous retrovirus retropepsin pseudogene 21 (Homo sapiens) (MER004403), endogenous retrovirus retropepsin pseudogene 22 (Homo sapiens chromosome X) (MER030287), subfamily A2A non-peptidase homologues (MER047046), subfamily A2A non-peptidase homologues (MER047052), subfamily A2A non-peptidase homologues (MER047076), subfamily A2A non-peptidase homologues (MER047080), subfamily A2A non-peptidase homologues (MER047088), subfamily A2A non-peptidase homologues (MER047089), subfamily A2A non-peptidase homologues (MER047091), subfamily A2A non-peptidase homologues (MER047092), subfamily A2A non-peptidase homologues (MER047093), subfamily A2A non-peptidase homologues (MER047094), subfamily A2A non-peptidase homologues (MER047097), subfamily A2A non-peptidase homologues (MER047099), subfamily A2A non-peptidase homologues MER047101), subfamily A2A non-peptidase homologues (MER047102), subfamily A2A non-peptidase homologues (MER047107), subfamily A2A non-peptidase homologues (MER047108), subfamily A2A non-peptidase homologues (MER047109), subfamily A2A non-peptidase homologues (MER047110), subfamily A2A non-peptidase homologues MER047111), subfamily A2A non-peptidase homologues (MER047114), subfamily A2A non-peptidase homologues (MER047118), subfamily A2A non-peptidase homologues (MER047121), subfamily A2A non-peptidase homologues (MER047122), subfamily A2A non-peptidase homologues (MER047126), subfamily A2A non-peptidase homologues (MER047129), subfamily A2A non-peptidase homologues (MER047130), subfamily A2A non-peptidase homologues (MER047134), subfamily A2A non-peptidase homologues (MER047135), subfamily A2A non-peptidase homologues (MER047137), subfamily A2A non-peptidase homologues (MER047140), subfamily A2A non-peptidase homologues (MER047141), subfamily A2A non-peptidase homologues (MER047142), subfamily A2A non-peptidase homologues (MER047148), subfamily A2A non-peptidase homologues (MER047149), subfamily A2A non-peptidase homologues (MER047151), subfamily A2A non-peptidase homologues (MER047154), subfamily A2A non-peptidase homologues (MER047155), subfamily A2A non-peptidase homologues (MER047156), subfamily A2A non-peptidase homologues (MER047157), subfamily A2A non-peptidase homologues (MER047159), subfamily A2A non-peptidase homologues (MER047161), subfamily A2A non-peptidase homologues (MER047163), subfamily A2A non-peptidase homologues (MER047166), subfamily A2A non-peptidase homologues (MER047171), subfamily A2A non-peptidase homologues (MER047173), subfamily A2A non-peptidase homologues (MER047174), subfamily A2A non-peptidase homologues (MER047179), subfamily A2A non-peptidase homologues (MER047183), subfamily A2A non-peptidase homologues (MER047186), subfamily A2A non-peptidase homologues (MER047190), subfamily A2A non-peptidase homologues (MER047191), subfamily A2A non-peptidase homologues (MER047196), subfamily A2A non-peptidase homologues (MER047198), subfamily A2A non-peptidase homologues (MER047199), subfamily A2A non-peptidase homologues (MER047201), subfamily A2A non-peptidase homologues (MER047202), subfamily A2A non-peptidase homologues (MER047203), subfamily A2A non-peptidase homologues (MER047204), subfamily A2A non-peptidase homologues (MER047205), subfamily A2A non-peptidase homologues (MER047207), subfamily A2A non-peptidase homologues (MER047208), subfamily A2A non-peptidase homologues (MER047210), subfamily A2A non-peptidase homologues (MER047211), subfamily A2A non-peptidase homologues (MER047212), subfamily A2A non-peptidase homologues (MER047213), subfamily A2A non-peptidase homologues (MER047215), subfamily A2A non-peptidase homologues (MER047216), subfamily A2A non-peptidase homologues (MER047218), subfamily A2A non-peptidase homologues (MER047219), subfamily A2A non-peptidase homologues (MER047221), subfamily A2A non-peptidase homologues (MER047224), subfamily A2A non-peptidase homologues (MER047225), subfamily A2A non-peptidase homologues (MER047226), subfamily A2A non-peptidase homologues (MER047227), subfamily A2A non-peptidase homologues (MER047230), subfamily A2A non-peptidase homologues (MER047232), subfamily A2A non-peptidase homologues (MER047233), subfamily A2A non-peptidase homologues (MER047234), subfamily A2A non-peptidase homologues (MER047236), subfamily A2A non-peptidase homologues (MER047238), subfamily A2A non-peptidase homologues (MER047239), subfamily A2A non-peptidase homologues (MER047240), subfamily A2A non-peptidase homologues (MER047242), subfamily A2A non-peptidase homologues (MER047243), subfamily A2A non-peptidase homologues (MER047249), subfamily A2A non-peptidase homologues (MER047251), subfamily A2A non-peptidase homologues (MER047252), subfamily A2A non-peptidase homologues (MER047254), subfamily A2A non-peptidase homologues (MER047255), subfamily A2A non-peptidase homologues (MER047263), subfamily A2A non-peptidase homologues (MER047265), subfamily A2A non-peptidase homologues (MER047266), subfamily A2A non-peptidase homologues (MER047267), subfamily A2A non-peptidase homologues (MER047268), subfamily A2A non-peptidase homologues (MER047269), subfamily A2A non-peptidase homologues (MER047272), subfamily A2A non-peptidase homologues (MER047273), subfamily A2A non-peptidase homologues (MER047274), subfamily A2A non-peptidase homologues (MER047275), subfamily A2A non-peptidase homologues (MER047276), subfamily A2A non-peptidase homologues (MER047279), subfamily A2A non-peptidase homologues (MER047280), subfamily A2A non-peptidase homologues (MER047281), subfamily A2A non-peptidase homologues (MER047282), subfamily A2A non-peptidase homologues (MER047284), subfamily A2A non-peptidase homologues (MER047285), subfamily A2A non-peptidase homologues (MER047289), subfamily A2A non-peptidase homologues (MER047290), subfamily A2A non-peptidase homologues (MER047294), subfamily A2A non-peptidase homologues (MER047295), subfamily A2A non-peptidase homologues (MER047298), subfamily A2A non-peptidase homologues (MER047300), subfamily A2A non-peptidase homologues (MER047302), subfamily A2A non-peptidase homologues (MER047304), subfamily A2A non-peptidase homologues (MER047305), subfamily A2A non-peptidase homologues (MER047306), subfamily A2A non-peptidase homologues (MER047307), subfamily A2A non-peptidase homologues (MER047310), subfamily A2A non-peptidase homologues (MER047311), subfamily A2A non-peptidase homologues (MER047314), subfamily A2A non-peptidase homologues (MER047318), subfamily A2A non-peptidase homologues (MER047320), subfamily A2A non-peptidase homologues (MER047321), subfamily A2A non-peptidase homologues (MER047322), subfamily A2A non-peptidase homologues (MER047326), subfamily A2A non-peptidase homologues (MER047327), subfamily A2A non-peptidase homologues (MER047330), subfamily A2A non-peptidase homologues (MER047333), subfamily A2A non-peptidase homologues (MER047362), subfamily A2A non-peptidase homologues (MER047366), subfamily A2A non-peptidase homologues (MER047369), subfamily A2A non-peptidase homologues (MER047370), subfamily A2A non-peptidase homologues (MER047371), subfamily A2A non-peptidase homologues (MER047375), subfamily A2A non-peptidase homologues (MER047376), subfamily A2A non-peptidase homologues (MER047381), subfamily A2A non-peptidase homologues (MER047383), subfamily A2A non-peptidase homologues (MER047384), subfamily A2A non-peptidase homologues (MER047385), subfamily A2A non-peptidase homologues (MER047388), subfamily A2A non-peptidase homologues (MER047389), subfamily A2A non-peptidase homologues (MER047391), subfamily A2A non-peptidase homologues (MER047394), subfamily A2A non-peptidase homologues (MER047396), subfamily A2A non-peptidase homologues (MER047400), subfamily A2A non-peptidase homologues (MER047401), subfamily A2A non-peptidase homologues (MER047403), subfamily A2A non-peptidase homologues (MER047406), subfamily A2A non-peptidase homologues (MER047407), subfamily A2A non-peptidase homologues (MER047410), subfamily A2A non-peptidase homologues (MER047411), subfamily A2A non-peptidase homologues (MER047413), subfamily A2A non-peptidase homologues (MER047414), subfamily A2A non-peptidase homologues (MER047416), subfamily A2A non-peptidase homologues (MER047417), subfamily A2A non-peptidase homologues (MER047420), subfamily A2A non-peptidase homologues (MER047423), subfamily A2A non-peptidase homologues (MER047424), subfamily A2A non-peptidase homologues (MER047428), subfamily A2A non-peptidase homologues (MER047429), subfamily A2A non-peptidase homologues (MER047431), subfamily A2A non-peptidase homologues (MER047434), subfamily A2A non-peptidase homologues (MER047439), subfamily A2A non-peptidase homologues (MER047442), subfamily A2A non-peptidase homologues (MER047445), subfamily A2A non-peptidase homologues (MER047449), subfamily A2A non-peptidase homologues (MER047450), subfamily A2A non-peptidase homologues (MER047452), subfamily A2A non-peptidase homologues (MER047455), subfamily A2A non-peptidase homologues (MER047457), subfamily A2A non-peptidase homologues (MER047458), subfamily A2A non-peptidase homologues (MER047459), subfamily A2A non-peptidase homologues (MER047463), subfamily A2A non-peptidase homologues (MER047468), subfamily A2A non-peptidase homologues (MER047469), subfamily A2A non-peptidase homologues (MER047470), subfamily A2A non-peptidase homologues (MER047476), subfamily A2A non-peptidase homologues (MER047478), subfamily A2A non-peptidase homologues (MER047483), subfamily A2A non-peptidase homologues (MER047488), subfamily A2A non-peptidase homologues (MER047489), subfamily A2A non-peptidase homologues (MER047490), subfamily A2A non-peptidase homologues (MER047493), subfamily A2A non-peptidase homologues (MER047494), subfamily A2A non-peptidase homologues (MER047495), subfamily A2A non-peptidase homologues (MER047496), subfamily A2A non-peptidase homologues (MER047497), subfamily A2A non-peptidase homologues (MER047499), subfamily A2A non-peptidase homologues (MER047502), subfamily A2A non-peptidase homologues (MER047504), subfamily A2A non-peptidase homologues (MER047511), subfamily A2A non-peptidase homologues (MER047513), subfamily A2A non-peptidase homologues (MER047514), subfamily A2A non-peptidase homologues (MER047515), subfamily A2A non-peptidase homologues (MER047516), subfamily A2A non-peptidase homologues (MER047520), subfamily A2A non-peptidase homologues (MER047533), subfamily A2A non-peptidase homologues (MER047537), subfamily A2A non-peptidase homologues (MER047569), subfamily A2A non-peptidase homologues (MER047570), subfamily A2A non-peptidase homologues (MER047584), subfamily A2A non-peptidase homologues (MER047603), subfamily A2A non-peptidase homologues (MER047604), subfamily A2A non-peptidase homologues (MER047606), subfamily A2A non-peptidase homologues (MER047609), subfamily A2A non-peptidase homologues (MER047616), subfamily A2A non-peptidase homologues (MER047619), subfamily A2A non-peptidase homologues (MER047648), subfamily A2A non-peptidase homologues (MER047649), subfamily A2A non-peptidase homologues (MER047662), subfamily A2A non-peptidase homologues (MER048004), subfamily A2A non-peptidase homologues (MER048018), subfamily A2A non-peptidase homologues (MER048019), subfamily A2A non-peptidase homologues (MER048023), subfamily A2A non-peptidase homologues (MER048037), subfamily A2A unassigned peptidases (MER047164), subfamily A2A unassigned peptidases (MER047231), subfamily A2A unassigned peptidases (MER047386), skin aspartic protease (MER057097), presenilin 1 (MER005221), presenilin 2 (MER005223), impas 1 peptidase (MER019701), impas 1 peptidase (MER184722), impas 4 peptidase (MER019715), impas 2 peptidase (MER019708), impas 5 peptidase (MER019712), impas 3 peptidase (MER019711), possible family A22 pseudogene (Homo sapiens chromosome 18) (MER029974), possible family A22 pseudogene (Homo sapiens chromosome 11) (MER023159), cathepsin V (MER004437), cathepsin X (MER004508), cathepsin F (MER004980), cathepsin L (MER000622), cathepsin S (MER000633), cathepsin O (MER001690), cathepsin K (MER000644), cathepsin W (MER003756), cathepsin H (MER000629), cathepsin B (MER000686), dipeptidyl-peptidase I (MER001937), bleomycin hydrolase (animal) (MER002481), tubulointerstitial nephritis antigen (MER016137), tubulointerstitial nephritis antigen-related protein (MER021799), cathepsin L-like pseudogene 1 (Homo sapiens) (MER002789), cathepsin B-like pseudogene (chromosome 4, Homo sapiens) (MER029469), cathepsin B-like pseudogene (chromosome 1, Homo sapiens) (MER029457), CTSLL2 g.p. (Homo sapiens) (MER005210), CTSLL3 g.p. (Homo sapiens) (MER005209), calpain-1 (MER000770), calpain-2 (MER000964), calpain-3 (MER001446), calpain-9 (MER004042), calpain-8 (MER021474), calpain-15 (MER004745), calpain-5 (MER002939), calpain-11 (MER005844), calpain-12 (MER029889), calpain-10 (MER013510), calpain-13 (MER020139), calpain-14 (MER029744), Mername-AA253 peptidase (MER005537), calpamodulin (MER000718), hypothetical protein 940251 (MER003201), ubiquitinyl hydrolase-L1 (MER000832), ubiquitinyl hydrolase-L3 (MER000836), ubiquitinyl hydrolase-BAP1 (MER003989), ubiquitinyl hydrolase-UCH37 (MER005539), ubiquitin-specific peptidase 5 (MER002066), ubiquitin-specific peptidase 6 (MER000863), ubiquitin-specific peptidase 4 (MER001795), ubiquitin-specific peptidase 8 (MER001884), ubiquitin-specific peptidase 13 (MER002627), ubiquitin-specific peptidase 2 (MER004834), ubiquitin-specific peptidase 11 (MER002693), ubiquitin-specific peptidase 14 (MER002667), ubiquitin-specific peptidase 7 (MER002896), ubiquitin-specific peptidase 9X (MER005877), ubiquitin-specific peptidase 10 (MER004439), ubiquitin-specific peptidase 1 (MER004978), ubiquitin-specific peptidase 12 (MER005454), ubiquitin-specific peptidase 16 (MER005493), ubiquitin-specific peptidase 15 (MER005427), ubiquitin-specific peptidase 17 (MER002900), ubiquitin-specific peptidase 19 (MER005428), ubiquitin-specific peptidase 20 (MER005494), ubiquitin-specific peptidase 3 (MER005513), ubiquitin-specific peptidase 9Y (MER004314), ubiquitin-specific peptidase 18 (MER005641), ubiquitin-specific peptidase 21 (MER006258), ubiquitin-specific peptidase 22 (MER012130), ubiquitin-specific peptidase 33 (MER014335), ubiquitin-specific peptidase 29 (MER012093), ubiquitin-specific peptidase 25 (MER011115), ubiquitin-specific peptidase 36 (MER014033), ubiquitin-specific peptidase 32 (MER014290), ubiquitin-specific peptidase 26 (Homo sapiens-type) (MER014292), ubiquitin-specific peptidase 24 (MER005706), ubiquitin-specific peptidase 42 (MER011852), ubiquitin-specific peptidase 46 (MER014629), ubiquitin-specific peptidase 37 (MER014633), ubiquitin-specific peptidase 28 (MER014634), ubiquitin-specific peptidase 47 (MER014636), ubiquitin-specific peptidase 38 (MER014637), ubiquitin-specific peptidase 44 (MER014638), ubiquitin-specific peptidase 50 (MER030315), ubiquitin-specific peptidase 35 (MER014646), ubiquitin-specific peptidase 30 (MER014649), Mername-AA091 peptidase (MER014743), ubiquitin-specific peptidase 45 (MER030314), ubiquitin-specific peptidase 51 (MER014769), ubiquitin-specific peptidase 34 (MER014780), ubiquitin-specific peptidase 48 (MER064620), ubiquitin-specific peptidase 40 (MER015483), ubiquitin-specific peptidase 41 (MER045268), ubiquitin-specific peptidase 31 (MER015493), Mername-AA129 peptidase (MER016485), ubiquitin-specific peptidase 49 (MER016486), Mername-AA187 peptidase (MER052579), USP17-like peptidase (MER030192), ubiquitin-specific peptidase 54 (MER028714), ubiquitin-specific peptidase 53 (MER027329), ubiquitin-specific endopeptidase 39 [misleading] (MER064621), Mername-AA090 non-peptidase homologue (MER014739), ubiquitin-specific peptidase 43 [misleading] (MER030140), ubiquitin-specific peptidase 52 [misleading] (MER030317), NEK2 pseudogene (MER014736), C19 pseudogene (Homo sapiens: chromosome 5) (MER029972), Mername-AA088 peptidase (MER014750), autophagin-2 (MER013564), autophagin-1 (MER013561), autophagin-3 (MER014316), autophagin-4 (MER064622), Cezanne deubiquitinylating peptidase (MER029042), Cezanne-2 peptidase (MER029044), tumor necrosis factor alpha-induced protein 3 (MER029050), trabid peptidase (MER029052), VCIP135 deubiquitinating peptidase (MER152304), otubain-1 (MER029056), otubain-2 (MER029061), CylD protein (MER030104), UfSP1 peptidase (MER042724), UfSP2 peptidase (MER060306), DUBA deubiquitinylating enzyme (MER086098), KIAA0459 (Homo sapiens)-like protein (MER122467), Otud1 protein (MER125457), glycosyltransferase 28 domain containing 1, isoform CRA_c (Homo sapiens)-like (MER123606), hin1L g.p. (Homo sapiens) (MER139816), ataxin-3 (MER099998), ATXN3L putative peptidase (MER115261), Josephin domain containing 1 (Homo sapiens) (MER125334), Josephin domain containing 2 (Homo sapiens) (MER124068), YOD1 peptidase (MER116559), legumain (plant alpha form) (MER044591), legumain (MER001800), glycosylphosphatidylinositol:protein transamidase (MER002479), legumain pseudogene (Homo sapiens) (MER029741), family C13 unassigned peptidases (MER175813), caspase-1 (MER000850), caspase-3 (MER000853), caspase-7 (MER002705), caspase-6 (MER002708), caspase-2 (MER001644), caspase-4 (MER001938), caspase-5 (MER002240), caspase-8 (MER002849), caspase-9 (MER002707), caspase-10 (MER002579), caspase-14 (MER012083), paracaspase (MER019325), Mername-AA143 peptidase (MER021304), Memame-AA186 peptidase (MER020516), putative caspase (Homo sapiens) (MER021463), FLIP protein (MER003026), Mername-AA142 protein (MER021316), caspase-12 pseudogene (Homo sapiens) (MER019698), Mername-AA093 caspase pseudogene (MER014766), subfamily C14A non-peptidase homologues (MER185329), subfamily C14A non-peptidase homologues (MER179956), separase (Homo sapiens-type) (MER011775), separase-like pseudogene (MER014797), SENP1 peptidase (MER011012), SENP3 peptidase (MER011019), SENP6 peptidase (MER011109), SENP2 peptidase (MER012183), SENP5 peptidase (MER014032), SENP7 peptidase (MER014095), SENP8 peptidase (MER016161), SENP4 peptidase (MER005557), pyroglutamyl-peptidase I (chordate) (MER011032), Mername-AA073 peptidase (MER029978), Sonic hedgehog protein (MER002539), Indian hedgehog protein (MER002538), Desert hedgehog protein (MER012170), dipeptidyl-peptidase III (MER004252), Mername-AA164 protein (MER020410), LOC138971 g.p. (Homo sapiens) (MER020074), Atp23 peptidase (MER060642), prenyl peptidase 1 (MER004246), aminopeptidase N (MER000997), aminopeptidase A (MER001012), leukotriene A4 hydrolase (MER001013), pyroglutamyl-peptidase II (MER012221), cytosol alanyl aminopeptidase (MER002746), cystinyl aminopeptidase (MER002060), aminopeptidase B (MER001494), aminopeptidase PILS (MER005331), arginyl aminopeptidase-like 1 (MER012271), leukocyte-derived arginine aminopeptidase (MER002968), aminopeptidase Q (MER052595), aminopeptidase O (MER019730), Tata binding protein associated factor (MER026493), angiotensin-converting enzyme peptidase unit 1 (MER004967), angiotensin-converting enzyme peptidase unit 2 (MER001019), angiotensin-converting enzyme-2 (MER011061), Memame-AA153 protein (MER020514), thimet oligopeptidase (MER001737), neurolysin (MER010991), mitochondrial intermediate peptidase (MER003665), Mername-AA154 protein (MER021317), leishmanolysin-2 (MER014492), leishmanolysin-3 (MER180031), matrix metallopeptidase-1 (MER001063), matrix metallopeptidase-8 (MER001084), matrix metallopeptidase-2 (MER001080), matrix metallopeptidase-9 (MER001085), matrix metallopeptidase-3 (MER001068), matrix metallopeptidase-10 (Homo sapiens-type) (MER001072), matrix metallopeptidase-11 (MER001075), matrix metallopeptidase-7 (MER001092), matrix metallopeptidase-12 (MER001089), matrix metallopeptidase-13 (MER001411), membrane-type matrix metallopeptidase-1 (MER001077), membrane-type matrix metallopeptidase-2 (MER002383), membrane-type matrix metallopeptidase-3 (MER002384), membrane-type matrix metallopeptidase-4 (MER002595), matrix metallopeptidase-20 (MER003021), matrix metallopeptidase-19 (MER002076), matrix metallopeptidase-23B (MER004766), membrane-type matrix metallopeptidase-5 (MER005638), membrane-type matrix metallopeptidase-6 (MER012071), matrix metallopeptidase-21 (MER006101), matrix metallopeptidase-22 (MER014098), matrix metallopeptidase-26 (MER012072), matrix metallopeptidase-28 (MER013587), matrix metallopeptidase-23A (MER037217), macrophage elastase homologue (chromosome 8, Homo sapiens) (MER030035), Memame-AA156 protein (MER021309), matrix metallopeptidase-like 1 (MER045280), subfamily M10A non-peptidase homologues (MER175912), subfamily M10A non-peptidase homologues (MER187997), subfamily M10A non-peptidase homologues (MER187998), subfamily M10A non-peptidase homologues (MER180000), meprin alpha subunit (MER001111), meprin beta subunit (MER005213), procollagen C-peptidase (MER001113), mammalian tolloid-like 1 protein (MER005124), mammalian-type tolloid-like 2 protein (MER005866), ADAMTS9 peptidase (MER012092), ADAMTS14 peptidase (MER016700), ADAMTS15 peptidase (MER017029), ADAMTS16 peptidase (MER015689), ADAMTS17 peptidase (MER016302), ADAMTS18 peptidase (MER016090), ADAMTS19 peptidase (MER015663), ADAM8 peptidase (MER003902), ADAM9 peptidase (MER001140), ADAM10 peptidase (MER002382), ADAM12 peptidase (MER005107), ADAM19 peptidase (MER012241), ADAM15 peptidase (MER002386), ADAM17 peptidase (MER003094), ADAM20 peptidase (MER004725), ADAMDEC1 peptidase (MER000743), ADAMTS3 peptidase (MER005100), ADAMTS4 peptidase (MER005101), ADAMTS1 peptidase (MER005546), ADAM28 peptidase (Homo sapiens-type) (MER005495), ADAMTS5 peptidase (MER005548), ADAMTS8 peptidase (MER005545), ADAMTS6 peptidase (MER005893), ADAMTS7 peptidase (MER005894), ADAM30 peptidase (MER006268), ADAM21 peptidase (Homo sapiens-type) (MER004726), ADAMTS10 peptidase (MER014331), ADAMTS12 peptidase (MER014337), ADAMTS13 peptidase (MER015450), ADAM33 peptidase (MER015143), ovastacin (MER029996), ADAMTS20 peptidase (Homo sapiens-type) (MER026906), procollagen I N-peptidase (MER004985), ADAM2 protein (MER003090), ADAM6 protein (MER047044), ADAM7 protein (MER005109), ADAM18 protein (MER012230), ADAM32 protein (MER026938), non-peptidase homologue (Homo sapiens chromosome 4) (MER029973), family M12 non-peptidase homologue (Homo sapiens chromosome 16) (MER047654), family M12 non-peptidase homologue (Homo sapiens chromosome 15) (MER047250), ADAM3B protein (Homo sapiens-type) (MER005199), ADAM11 protein (MER001146), ADAM22 protein (MER005102), ADAM23 protein (MER005103), ADAM29 protein (MER006267), protein similar to ADAM21 peptidase preproprotein (Homo sapiens) (MER026944), Mername-AA225 peptidase homologue (Homo sapiens) (MER047474), putative ADAM pseudogene (chromosome 4, Homo sapiens) (MER029975), ADAM3A g.p. (Homo sapiens) (MER005200), ADAM1 g.p. (Homo sapiens) (MER003912), subfamily M12B non-peptidase homologues (MER188210), subfamily M12B non-peptidase homologues (MER188211), subfamily M12B non-peptidase homologues (MER188212), subfamily M12B non-peptidase homologues (MER188220), neprilysin (MER001050), endothelin-converting enzyme 1 (MER001057), endothelin-converting enzyme 2 (MER004776), DINE peptidase (MER005197), neprilysin-2 (MER013406), Kell blood-group protein (MER001054), PHEX peptidase (MER002062), i-AAA peptidase (MER001246), i-AAA peptidase (MER005755), paraplegin (MER004454), Afg3-like protein 2 (MER005496), Afg3-like protein 1A (MER014306), pappalysin-1 (MER002217), pappalysin-2 (MER014521), farnesylated-protein converting enzyme 1 (MER002646), metalloprotease-related protein-1 (MER030873), aminopeptidase AMZ2 (MER011907), aminopeptidase AMZ1 (MER058242), carboxypeptidase A1 (MER001190), carboxypeptidase A2 (MER001608), carboxypeptidase B (MER001194), carboxypeptidase N (MER001198), carboxypeptidase E (MER001199), carboxypeptidase M (MER001205), carboxypeptidase U (MER001193), carboxypeptidase A3 (MER001187), metallocarboxypeptidase D peptidase unit 1 (MER003781), metallocarboxypeptidase Z (MER003428), metallocarboxypeptidase D peptidase unit 2 (MER004963), carboxypeptidase A4 (MER013421), carboxypeptidase A6 (MER013456), carboxypeptidase A5 (MER017121), metallocarboxypeptidase O (MER016044), cytosolic carboxypeptidase-like protein 5 (MER033174), cytosolic carboxypeptidase 3 (MER033176), cytosolic carboxypeptidase 6 (MER033178), cytosolic carboxypeptidase 1 (MER033179), cytosolic carboxypeptidase 2 (MER037713), metallocarboxypeptidase D non-peptidase unit (MER004964), adipocyte-enhancer binding protein 1 (MER003889), carboxypeptidase-like protein X1 (MER013404), carboxypeptidase-like protein X2 (MER078764), cytosolic carboxypeptidase (MER026952), family M14 non-peptidase homologues (MER199530), insulysin (MER001214), mitochondrial processing peptidase beta-subunit (MER004497), nardilysin (MER003883), eupitrilysin (MER004877), mitochondrial processing peptidase non-peptidase alpha subunit (MER001413), ubiquinol-cytochrome c reductase core protein I (MER003543), ubiquinol-cytochrome c reductase core protein II (MER003544), ubiquinol-cytochrome c reductase core protein domain 2 (MER043998), insulysin unit 2 (MER046821), nardilysin unit 2 (MER046874), insulysin unit 3 (MER078753), mitochondrial processing peptidase subunit alpha unit 2 (MER124489), nardilysin unit 3 (MER142856), LOC133083 g.p. (Homo sapiens) (MER021876), subfamily M16B non-peptidase homologues (MER188757), leucyl aminopeptidase (animal) (MER003100), Memame-AA040 peptidase (MER003919), leucyl aminopeptidase-1 (Caenorhabditis-type) (MER013416), methionyl aminopeptidase 1 (MER001342), methionyl aminopeptidase 2 (MER001728), aminopeptidase P2 (MER004498), Xaa-Pro dipeptidase (eukaryote) (MER001248), aminopeptidase P1 (MER004321), mitochondrial intermediate cleaving peptidase 55 kDa (MER013463), mitochondrial methionyl aminopeptidase (MER014055), Memame-AA020 peptidase homologue (MER010972), proliferation-association protein 1 (MER005497), chromatin-specific transcription elongation factor 140 kDa subunit (MER026495), proliferation-associated protein 1-like (Homo sapiens chromosome X) (MER029983), Mername-AA226 peptidase homologue (Homo sapiens) (MER056262), Mername-AA227 peptidase homologue (Homo sapiens) (MER047299), subfamily M24A non-peptidase homologues (MER179893), aspartyl aminopeptidase (MER003373), Gly-Xaa carboxypeptidase (MER033182), camosine dipeptidase II (MER014551), carnosine dipeptidase I (MER015142), Mername-AA161 protein (MER021873), aminoacylase (MER001271), glutamate carboxypeptidase II (MER002104), NAALADASE L peptidase (MER005239), glutamate carboxypeptidase III (MER005238), plasma glutamate carboxypeptidase (MER005244), Mername-AA103 peptidase (MER015091), Fxna peptidase (MER029965), transferrin receptor protein (MER002105), transferrin receptor 2 protein (MER005152), glutaminyl cyclise (MER015095), glutamate carboxypeptidase II (Homo sapiens)-type non-peptidase homologue (MER026971), nicalin (MER044627), membrane dipeptidase (MER001260), membrane-bound dipeptidase-2 (MER013499), membrane-bound dipeptidase-3 (MER013496), dihydro-orotase (MER005767), dihydropyrimidinase (MER033266), dihydropyrimidinase related protein-1 (MER030143), dihydropyrimidinase related protein-2 (MER030155), dihydropyrimidinase related protein-3 (MER030151), dihydropyrimidinase related protein-4 (MER030149), dihydropyrimidinase related protein-5 (MER030136), hypothetical protein like 5730457F11RIK (MER033184), 1300019j08rik protein (MER033186)), guanine aminohydrolase (MER037714), Kael putative peptidase (MER001577), OSGEPL1-like protein (MER013498), S2P peptidase (MER004458), subfamily M23B non-peptidase homologues (MER199845), subfamily M23B non-peptidase homologues (MER199846), subfamily M23B non-peptidase homologues (MER199847), subfamily M23B non-peptidase homologues (MER137320), subfamily M23B non-peptidase homologues (MER201557), subfamily M23B non-peptidase homologues (MER199417), subfamily M23B non-peptidase homologues (MER199418), subfamily M23B non-peptidase homologues (MER199419), subfamily M23B non-peptidase homologues (MER199420), subfamily M23B non-peptidase homologues (MER175932), subfamily M23B non-peptidase homologues (MER199665), Pohl peptidase (MER020382), Jab1/MPN domain metalloenzyme (MER022057), Mername-AA165 peptidase (MER021865), Brcc36 isopeptidase (MER021890), histone H2A deubiquitinase MYSM1 (MER021887), AMSH deubiquitinating peptidase (MER030146), putative peptidase (Homo sapiens chromosome 2) (MER029970), Mername-AA168 protein (MER021886), COP9 signalosome subunit 6 (MER030137), 26S proteasome non-ATPase regulatory subunit 7 (MER030134), eukaryotic translation initiation factor 3 subunit 5 (MER030133), IFP38 peptidase homologue (MER030132), subfamily M67A non-peptidase homologues (MER191181), subfamily M67A unassigned peptidases (MER191144), granzyme B (Homo sapiens-type) (MER000168), testisin (MER005212), tryptase beta (MER000136), kallikrein-related peptidase 5 (MER005544), corin (MER005881), kallikrein-related peptidase 12 (MER006038), DESC1 peptidase (MER006298), tryptase gamma 1 (MER011036), kallikrein-related peptidase 14 (MER011038), hyaluronan-binding peptidase (MER003612), transmembrane peptidase, serine 4 (MER011104), intestinal serine peptidase (rodent) (MER016130), adrenal secretory serine peptidase (MER003734), tryptase delta 1 (Homo sapiens) (MER005948), matriptase-3 (MER029902), marapsin (MER006119), tryptase-6 (MER006118), ovochymase-1 domain 1 (MER099182), transmembrane peptidase, serine 3 (MER005926), kallikrein-related peptidase 15 (MER000064), Mername-AA031 peptidase (MER014054), TMPRSS13 peptidase (MER014226), Memame-AA038 peptidase (MER062848), Mername-AA204 peptidase (MER029980), cationic trypsin (Homo sapiens-type) (MER000020), elastase-2 (MER000118), mannan-binding lectin-associated serine peptidase-3 (MER031968), cathepsin G (MER000082), myeloblastin (MER000170), granzyme A (MER001379), granzyme M (MER001541), chymase (Homo sapiens-type) (MER000123), tryptase alpha (MER000135), granzyme K (MER001936), granzyme H (MER000166), chymotrypsin B (MER000001), elastase-1 (MER003733), pancreatic endopeptidase E (MER000149), pancreatic elastase II (MER000146), enteropeptidase (MER002068), chymotrypsin C (MER000761), prostasin (MER002460), kallikrein 1 (MER000093), kallikrein-related peptidase 2 (MER000094), kallikrein-related peptidase 3 (MER000115), mesotrypsin (MER000022), complement component C1r-like peptidase (MER016352), complement factor D (MER000130), complement component activated C1r (MER000238), complement component activated CIs (MER000239), complement component C2a (MER000231), complement factor B (MER000229), mannan-binding lectin-associated serine peptidase 1 (MER000244), complement factor I (MER000228), pancreatic endopeptidase E form B (MER000150), pancreatic elastase IIB (MER000147), coagulation factor XIIa (MER000187), plasma kallikrein (MER000203) coagulation factor Xia (MER000210), coagulation factor IXa (MER000216), coagulation factor Vila (MER000215), coagulation factor Xa (MER000212), thrombin (MER000188), protein C (activated) (MER000222), acrosin (MER000078), hepsin (MER000156), hepatocyte growth factor activator (MER000186), mannan-binding lectin-associated serine peptidase 2 (MER002758), u-plasminogen activator (MER000195), t-plasminogen activator (MER000192), plasmin (MER000175), kallikrein-related peptidase 6 (MER002580), neurotrypsin (MER004171), kallikrein-related peptidase 8 (MER005400), kallikrein-related peptidase 10 (MER003645), epitheliasin (MER003736), kallikrein-related peptidase 4 (MER005266), prosemin (MER004214), chymopasin (MER001503), kallikrein-related peptidase 11 (MER004861), kallikrein-related peptidase 11 (MER216142), trypsin-2 type A (MER000021), HtrA1 peptidase (Homo sapiens-type) (MER002577), HtrA2 peptidase (MER208413), HtrA2 peptidase (MER004093), HtrA3 peptidase (MER014795), HtrA4 peptidase (MER016351), Tysndl peptidase (MER050461), TMPRSS12 peptidase (MER017085), HAT-like putative peptidase 2 (MER021884), trypsin C (MER021898), kallikrein-related peptidase 7 (MER002001), matriptase (MER003735), kallikrein-related peptidase 13 (MER005269), kallikrein-related peptidase 9 (MER005270), matriptase-2 (MER005278), umbilical vein peptidase (MER005421), LCLP peptidase (MER001900), spinesin (MER014385), marapsin-2 (MER021929), complement factor D-like putative peptidase (MER056164), ovochymase-2 (MER022410), HAT-like 4 peptidase (MER044589), ovochymase 1 domain 1 (MER022412), epidermis-specific SP-like putative peptidase (MER029900), testis serine peptidase 5 (MER029901), Mername-AA258 peptidase (MER000285), polyserase-IA unit 1 (MER030879), polyserase-IA unit 2 (MER030880), testis serine peptidase 2 (human-type) (MER033187), hypothetical acrosin-like peptidase (Homo sapiens) (MER033253), HAT-like 5 peptidase (MER028215), polyserase-3 unit 1 (MER061763), polyserase-3 unit 2 (MER061748), peptidase similar to tryptophan/serine protease (MER056263), polyserase-2 unit 1 (MER061777), Mername-AA123 peptidase (MER021930), HAT-like 2 peptidase (MER099184), hCG2041452-like protein (MER099172), hCG22067 (Homo sapiens) (MER099169), brain-rescue-factor-1 (Homo sapiens) (MER098873), hCG2041108 (Homo sapiens) (MER099173), polyserase-2 unit 2 (MER061760), polyserase-2 unit 3 (MER065694), Mername-AA201 (peptidase homologue) MER099175, secreted trypsin-like serine peptidase homologue (MER030000), polyserase-1A unit 3 (MER029880), azurocidin (MER000119), haptoglobin-1 (MER000233), haptoglobin-related protein (MER000235), macrophage-stimulating protein (MER001546), hepatocyte growth factor (MER000185), protein Z (MER000227), TESP1 protein (MER047214), LOC136242 protein (MER016132), plasma kallikrein-like protein 4 (MER016346), PRSS35 protein (MER016350), DKFZp586H2123-like protein (MER066474), apolipoprotein (MER000183), psi-KLK1 pseudogene (Homo sapiens) (MER033287), tryptase pseudogene I (MER015077), tryptase pseudogene II (MER015078), tryptase pseudogene III (MER015079), subfamily S1A unassigned peptidases (MER216982), subfamily S1A unassigned peptidases (MER216148), amidophosphoribosyltransferase precursor (MER003314), glutamine-fructose-6-phosphate transaminase 1 (MER003322), glutamine:fructose-6-phosphate amidotransferase (MER012158), Memame-AA144 protein (MER021319), asparagine synthetase (MER033254), family C44 non-peptidase homologues (MER159286), family C44 unassigned peptidases (MER185625) family C44 unassigned peptidases (MER185626), secernin 1 (MER045376), secernin 2 (MER064573), secernin 3 (MER064582), acid ceramidase precursor (MER100794), N-acylethanolamine acid amidase precursor (MER141667), proteasome catalytic subunit 1 (MER000556), proteasome catalytic subunit 2 (MER002625), proteasome catalytic subunit 3 (MER002149), proteasome catalytic subunit 1i (MER000552), proteasome catalytic subunit 2i (MER001515), proteasome catalytic subunit 3i (MER000555), proteasome catalytic subunit 5t (MER026203), protein serine kinase c17 (MER026497), proteasome subunit alpha 6 (MER000557), proteasome subunit alpha 2 (MER000550), proteasome subunit alpha 4 (MER000554), proteasome subunit alpha 7 (MER033250), proteasome subunit alpha 5 (MER000558), proteasome subunit alpha 1 (MER000549), proteasome subunit alpha 3 (MER000553), proteasome subunit XAPC7 (MER004372), proteasome subunit beta 3 (MER001710), proteasome subunit beta 2 (MER002676), proteasome subunit beta 1 (MER000551), proteasome subunit beta 4 (MER001711), Memame-AA230 peptidase homologue (Homo sapiens) (MER047329), Memame-AA231 pseudogene (Homo sapiens) (MER047172), Memame-AA232 pseudogene (Homo sapiens) (MER047316), glycosylasparaginase precursor (MER003299), isoaspartyl dipeptidase (threonine type) (MER031622), taspase-1 (MER016969), gamma-glutamyltransferase 5 (mammalian-type) (MER001977), gamma-glutamyltransferase 1 (mammalian-type) (MER001629), gamma-glutamyltransferase 2 (Homo sapiens) (MER001976), gamma-glutamyltransferase-like protein 4 (MER002721), gamma-glutamyltransferase-like protein 3 (MER016970), similar to gamma-glutamyltransferase 1 precursor (Homo sapiens) (MER026204), similar to gamma-glutamyltransferase 1 precursor (Homo sapiens) (MER026205), Memame-AA211 putative peptidase (MER026207), gamma-glutamyltransferase 6 (MER159283), gamma-glutamyl transpeptidase homologue (chromosome 2, Homo sapiens) (MER037241), polycystin-1 (MER126824), KIAA1879 protein (MER159329), polycystic kidney disease 1-like 3 (MER172554), gamma-glutamyl hydrolase (MER002963), guanine 5″-monophosphate synthetase (MER043387), carbamoyl-phosphate synthase (Homo sapiens-type) (MER078640), dihydro-orotase (N-terminal unit) (Homo sapiens-type) (MER060647), DJ-1 putative peptidase (MER003390), Mername-AA100 putative peptidase (MER014802), Mername-AA101 non-peptidase homologue (MER014803), KIAA0361 protein (Homo sapiens-type) (MER042827), F1134283 protein (Homo sapiens) (MER044553), non-peptidase homologue chromosome 21 open reading frame 33 (Homo sapiens) (MER160094), family C56 non-peptidase homologues (MER177016), family C56 non-peptidase homologues (MER176613), family C56 non-peptidase homologues (MER176918), EGF-like module containing mucin-like hormone receptor-like 2 (MER037230), CD97 antigen (human type) (MER037286), EGF-like module containing mucin-like hormone receptor-like 3 (MER037288), EGF-like module containing mucin-like hormone receptor-like 1 (MER037278), EGF-like module containing mucin-like hormone receptor-like 4 (MER037294), cadherin EGF LAG seven-pass G-type receptor 2 precursor (Homo sapiens) (MER045397), Gpr64 (Mus musculus)-type protein (MER123205), GPR56 (Homo sapiens)-type protein (MER122057), latrophilin 2 (MER122199), latrophilin-1 (MER126380), latrophilin 3 (MER124612), protocadherin Flamingo 2 (MER124239), ETL protein (MER126267), G protein-coupled receptor 112 (MER126114), seven transmembrane helix receptor (MER125448), Gpr114 protein (MER159320), GPR126 vascular inducible G protein-coupled receptor (MER140015), GPR125 (Homo sapiens)-type protein (MER159279), GPR116 (Homo sapiens)-type G-protein coupled receptor (MER159280), GPR128 (Homo sapiens)-type G-protein coupled receptor (MER162015), GPR133 (Homo sapiens)-type protein (MER159334), GPR110 G-protein coupled receptor (MER159277), GPR97 protein (MER159322), KPG 006 protein (MER161773), KPG 008 protein (MER161835), KPG_009 protein (MER159335), unassigned homologue (MER166269), GPR113 protein (MER159352), brain-specific angiogenesis inhibitor 2 (MER159746), PIDD auto-processing protein unit 1 (MER020001), PIDD auto-processing protein unit 2 (MER063690), MUC1 self-cleaving mucin (MER074260), dystroglycan (MER054741), proprotein convertase 9 (MER022416), site-1 peptidase (MER001948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non-peptidase homologues (MER201339), subfamily S8A non-peptidase homologues (MER191613), subfamily S8A unassigned peptidases (MER191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MER017240), Mername-AA194 putative peptidase (MER017353), Memame-AA195 putative peptidase (MER017367), Mername-AA196 putative peptidase (MER017368), Memame-AA197 putative peptidase (MER017371), C14orf29 protein (MER033244), hypothetical protein (MER033245), hypothetical esterase/lipase/thioesterase (MER047309), protein bat5 (MER037840), hypothetical protein flj40219 (MER033212), hypothetical protein flj37464 (MER033240), hypothetical protein flj33678 (MER033241), dipeptidylpeptidase homologue DPP6 (MER000403), dipeptidylpeptidase homologue DPP10 (MER005988), protein similar to Mus musculus chromosome 20 open reading frame 135 (MER037845), kynurenine formamidase (MER046020), thyroglobulin precursor (MER011604), acetylcholinesterase (MER033188), cholinesterase (MER033198), carboxylesterase D1 (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA1363-like protein (MER033242), hormone-sensitive lipase (MER033274), neuroligin 1 (MER033280), neuroligin 2 (MER033283), family S9 non-peptidase homologues (MER212939), family S9 non-peptidase homologues (MER211490), subfamily S9C unassigned peptidases (MER192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MER010960), family S15 unassigned peptidases (MER199442), family S15 unassigned peptidases (MER200437), family S15 unassigned peptidases (MER212825), lysosomal Pro-Xaa carboxypeptidase (MER000446), dipeptidyl-peptidase II (MER004952), thymus-specific serine peptidase (MER005538), epoxide hydrolase-like putative peptidase (MER031614), Loc328574-like protein (MER033246), abhydrolase domain-containing protein 4 (MER031616), epoxide hydrolase (MER000432), mesoderm specific transcript protein (MER199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein 922408 (epoxide hydrolase) (MER031617), monoglyceride lipase (MER033247), hypothetical protein (MER033249), valacyclovir hydrolase (MER033259), Ccg1-interacting factor b (MER210738), glycosylasparaginase precursor (MER003299), isoaspartyl dipeptidase (threonine type) (MER031622). taspase-1 (MER016969), gamma-glutamyltransferase 5 (mammalian-type) (MER001977), gamma-glutamyltransferase 1 (mammalian-type) (MER001629), gamma-glutamyltransferase 2 (Homo sapiens) (MER001976), gamma-glutamyltransferase-like protein 4 (MER002721). gamma-glutamyltransferase-like protein 3 (MER016970). similar to gamma-glutamyltransferase 1 precursor (Homo sapiens) (MER026204). similar to gamma-glutamyltransferase 1 precursor (Homo sapiens) (MER026205). Mername-AA211 putative peptidase (MER026207). gamma-glutamyltransferase 6 (MER159283). gamma-glutamyl transpeptidase homologue (chromosome 2, Homo sapiens) (MER037241). polycystin-1 (MER126824), KIAA1879 protein (MER159329). polycystic kidney disease 1-like 3 (MER172554). gamma-glutamyl hydrolase (MER002963). guanine 5″-monophosphate synthetase (MER043387). carbamoyl-phosphate synthase (Homo sapiens-type) (MER078640). dihydro-orotase (N-terminal unit) (Homo sapiens-type) (MER060647). DJ-1 putative peptidase (MER003390). Memame-AA100 putative peptidase (MER014802). Mername-AA101 non-peptidase homologue (MER014803). KIAA0361 protein (Homo sapiens-type) (MER042827). F1134283 protein (Homo sapiens) (MER044553). non-peptidase homologue chromosome 21 open reading frame 33 (Homo sapiens) (MER160094). family C56 non-peptidase homologues (MER177016), family C56 non-peptidase homologues (MER176613). family C56 non-peptidase homologues (MER176918). EGF-like module containing mucin-like hormone receptor-like 2 (MER037230). CD97 antigen (human type) (MER037286). EGF-like module containing mucin-like hormone receptor-like 3 (MER037288). EGF-like module containing mucin-like hormone receptor-like 1 (MER037278). EGF-like module containing mucin-like hormone receptor-like 4 (MER037294). cadherin EGF LAG seven-pass G-type receptor 2 precursor (Homo sapiens) (MER045397), Gpr64 (Mus musculus)-type protein (MER123205). GPR56 (Homo sapiens)-type protein (MER122057). latrophilin 2 (MER122199). latrophilin-1 (MER126380). latrophilin 3 (MER124612). protocadherin Flamingo 2 (MER124239). ETL protein (MER126267). G protein-coupled receptor 112 (MER126114). seven transmembrane helix receptor (MER125448). Gpr114 protein (MER159320). GPR126 vascular inducible G protein-coupled receptor (MER140015). GPR125 (Homo sapiens)-type protein (MER159279). GPR116 (Homo sapiens)-type G-protein coupled receptor (MER159280). GPR128 (Homo sapiens)-type G-protein coupled receptor (MER162015). GPR133 (Homo sapiens)-type protein (MER159334) GPR110 G-protein coupled receptor (MER159277), GPR97 protein (MER159322), KPG_006 protein (MER161773) KPG 008 protein (MER161835), KPG 009 protein (MER159335), unassigned homologue (MER166269), GPR113 protein (MER159352), brain-specific angiogenesis inhibitor 2 (MER159746), PIDD auto-processing protein unit 1 (MER020001), PIDD auto-processing protein unit 2 (MER063690), MUC1 self-cleaving mucin (MER074260), dystroglycan (MER054741), proprotein convertase 9 (MER022416), site-1 peptidase (MER001948), furin (MER000375), proprotein convertase 1 (MER000376), proprotein convertase 2 (MER000377), proprotein convertase 4 (MER028255), PACE4 proprotein convertase (MER000383), proprotein convertase 5 (MER002578), proprotein convertase 7 (MER002984), tripeptidyl-peptidase II (MER000355), subfamily S8A non-peptidase homologues (MER201339), subfamily S8A non-peptidase homologues (MER191613), subfamily S8A unassigned peptidases (MER191611), subfamily S8A unassigned peptidases (MER191612), subfamily S8A unassigned peptidases (MER191614), tripeptidyl-peptidase I (MER003575), prolyl oligopeptidase (MER000393), dipeptidyl-peptidase IV (eukaryote) (MER000401), acylaminoacyl-peptidase (MER000408), fibroblast activation protein alpha subunit (MER000399), PREPL A protein (MER004227), dipeptidyl-peptidase 8 (MER013484), dipeptidyl-peptidase 9 (MER004923), FLJ1 putative peptidase (MER017240), Memame-AA194 putative peptidase (MER017353), Memame-AA195 putative peptidase (MER017367), Mername-AA196 putative peptidase (MER017368), Mername-AA197 putative peptidase (MER017371), C14orf29 protein (MER033244), hypothetical protein (MER033245), hypothetical esterase/lipase/thioesterase (MER047309), protein bat5 (MER037840), hypothetical protein flj40219 (MER033212), hypothetical protein flj37464 (MER033240), hypothetical protein flj33678 (MER033241), dipeptidylpeptidase homologue DPP6 (MER000403), dipeptidylpeptidase homologue DPP10 (MER005988), protein similar to Mus musculus chromosome 20 open reading frame 135 (MER037845), kynurenine formamidase (MER046020), thyroglobulin precursor (MER011604), acetylcholinesterase (MER033188), cholinesterase (MER033198), carboxylesterase D1 (MER033213), liver carboxylesterase (MER033220), carboxylesterase 3 (MER033224), carboxylesterase 2 (MER033226), bile salt-dependent lipase (MER033227), carboxylesterase-related protein (MER033231), neuroligin 3 (MER033232), neuroligin 4, X-linked (MER033235), neuroligin 4, Y-linked (MER033236), esterase D (MER043126), arylacetamide deacetylase (MER033237), KIAA1363-like protein (MER033242), hormone-sensitive lipase (MER033274), neuroligin 1 (MER033280), neuroligin 2 (MER033283), family S9 non-peptidase homologues (MER212939), family S9 non-peptidase homologues (MER211490), subfamily S9C unassigned peptidases (MER192341), family S9 unassigned peptidases (MER209181), family S9 unassigned peptidases (MER200434), family S9 unassigned peptidases (MER209507), family S9 unassigned peptidases (MER209142), serine carboxypeptidase A (MER000430), vitellogenic carboxypeptidase-like protein (MER005492), RISC peptidase (MER010960), family S15 unassigned peptidases (MER199442), family S15 unassigned peptidases (MER200437), family S15 unassigned peptidases (MER212825), lysosomal Pro-Xaa carboxypeptidase (MER000446), dipeptidyl-peptidase II (MER004952), thymus-specific serine peptidase (MER005538), epoxide hydrolase-like putative peptidase (MER031614), Loc328574-like protein (MER033246), abhydrolase domain-containing protein 4 (MER031616), epoxide hydrolase (MER000432), mesoderm specific transcript protein (MER199890), mesoderm specific transcript protein (MER017123), cytosolic epoxide hydrolase (MER029997), cytosolic epoxide hydrolase (MER213866), similar to hypothetical protein FLJ22408 (MER031608), CGI-58 putative peptidase (MER030163), Williams-Beuren syndrome critical region protein 21 epoxide hydrolase (MER031610), epoxide hydrolase (MER031612), hypothetical protein flj22408 (epoxide hydrolase) (MER031617), monoglyceride lipase (MER033247), hypothetical protein (MER033249), valacyclovir hydrolase (MER033259), Ccg1-interacting factor b (MER210738).

Protease enzymatic activity can be regulated. For example, certain proteases can be inactivated by the presence or absence of a specific agent (e.g., that binds to the protease, such as specific small molecule inhibitors). Such proteases can be referred to as a “repressible protease.” Exemplary inhibitors for certain proteases are listed in Table 4B. For example, an NS3 protease can be repressed by a protease inhibitor including, but not limited to, simeprevir, danoprevir, asunaprevir, ciluprevir, boceprevir, sovaprevir, paritaprevir, telaprevir, grazoprevir, glecaprevir, and voxiloprevir. In another example, protease activity can be regulated through regulating expression of the protease itself, such as engineering a cell to express a protease using an inducible promoter system (e.g., Tet On/Off systems) or cell-specific promoters (promoters that can be used to express a heterologous protease are described in more detail in the Section herein titled “Promoters”). A protease can also contain a degron, such as any of the degrons described herein, and can be regulated using any of the degron systems described herein.

Protease enzymatic activity can also be regulated through selection of a specific protease cleavage site. For example, a protease cleavage site can be selected and/or engineered such that the sequence demonstrates a desired rate-of-cleavage by a desired protease, such as reduced cleavage kinetics relative to an endogenous sequence of a substrate naturally cleaved by the desired protease. As another example, a protease cleavage site can be selected and/or engineered such that the sequence demonstrates a desired rate-of-cleavage in a cell-state specific manner. For example, various cell states (e.g., following cellular signaling, such as immune cell activation) can influence the expression and/or localization of certain proteases. As an illustrative example, ADAM17 protein levels and localization is known to be influenced by signaling, such as through Protein kinase C (PKC) signaling pathways (e.g., activation by the PKC activator Phorbol-12-myristat-13-acetat [PMA]). Accordingly, a protease cleavage site can be selected and/or engineered such that cleavage of the protease cleavage site and subsequent release of an effector molecule is increased or decreased, as desired, depending on the protease properties (e.g., expression and/or localization) in a specific cell state. As another example, a protease cleavage site (particularly in combination with a specific membrane tethering domain) can be selected and/or engineered for optimal protein expression of the chimeric protein.

### Cell Membrane Tethering Domain

The membrane-cleavable chimeric proteins provided for herein include a cell-membrane tethering domain (referred to as “MT” in the formula S-C-MT or MT-C-S). In general, the cell-membrane tethering domain can be any amino acid sequence motif capable of directing the chimeric protein to be localized to (e.g., inserted into), or otherwise associated with, the cell membrane of the cell expressing the chimeric protein. The cell-membrane tethering domain can be a transmembrane-intracellular domain. The cell-membrane tethering domain can be a transmembrane domain. The cell-membrane tethering domain can be an integral membrane protein domain (e.g., a transmembrane domain). The cell-membrane tethering domain can be derived from a Type I, Type II, or Type III transmembrane protein. The cell-membrane tethering domain can include post-translational modification tag, or motif capable of post-translational modification to modify the chimeric protein to include a post-translational modification tag, where the post-translational modification tag allows association with a cell membrane. Examples of post-translational modification tags include, but are not limited to, lipid-anchor domains (e.g., a GPI lipid-anchor, a myristoylation tag, or palmitoylation tag). Examples of cell-membrane tethering domains include, but are not limited to, a transmembrane-intracellular domain and/or transmembrane domain derived from PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, or BTLA. The cell membrane tethering domain can be a cell surface receptor or a cell membrane-bound portion thereof. Sequences of exemplary cell membrane tethering domains are provided in Table 4C.

TABLE 4C

| | Amino Acid | DNA |
|---|---|---|
| Source | Sequence | Sequence |
| B7-1 | LLPSWAITLISVN | CTGCTGCCAAGCTGGGCCATCA |
| | GIFVICCLTYCFA | CACTGATCTCCGTGAACGGCAT |
| | PRCRERRRNERLR | CTTCGTGATCTGTTGCCTGACC |
| | RESVRPV | TACTGCTTCGCCCCTCGGTGCA |
| | (SEQ ID | GAGAGCGGAGAAGAAACGAACG |
| | NO: 219) | GCTGCGGAGAGAATCTGTGCGG |
| | | CCTGTG |
| | | (SEQ ID NO: 220) |
| | | OR |
| | | CTGCTGCCTAGCTGGGCCATCA |
| | | CACTGATCTCCGTGAACGGCAT |
| | | CTTCGTGATCTGCTGCCTGACC |
| | | TACTGCTTCGCCCCTAGATGCA |
| | | GAGAGCGGCGGAGAAACGAACG |
| | | GCTGAGAAGAGAATCTGTGCGG |
| | | CCCGTT |
| | | (SEQ ID NO: 331) |

In general, for all membrane-cleavable chimeric proteins described herein, the cell membrane tethering domain is either: (1) C-terminal of the protease cleavage site and N-terminal of any intracellular domain, if present (in other words, the cell membrane tethering domain is in between the protease cleavage site and, if present, an intracellular domain); or (2) N-terminal of the protease cleavage site and C-terminal of any intracellular domain, if present (also between the protease cleavage site and, if present, an intracellular domain with domain orientation inverted). In embodiments featuring a degron associated with the chimeric protein, the degron domain is the terminal cytoplasmic-oriented domain, specifically relative to the cell membrane tethering (in other words, the cell membrane tethering domain is in between the protease cleavage site and the degron). The cell membrane tethering domain can be connected to the protease cleavage site by a polypeptide linker, i.e., a polypeptide sequence not generally considered to be part of cell membrane tethering domain or protease cleavage site. The cell membrane tethering domain can be connected to an intracellular domain, if present, by a polypeptide linker, i.e., a polypeptide sequence not generally considered to be part of the cell membrane tethering domain or the intracellular domain. The cell membrane tethering domain can be connected to the degron, if present, by a polypeptide linker, i.e., a polypeptide sequence not generally considered to be part of the cell membrane tethering domain or degron. A polypeptide linker can be any amino acid sequence that connects a first polypeptide sequence and a second polypeptide sequence. A polypeptide linker can be a flexible linker (e.g., a Gly-Ser-Gly sequence). Examples of polypeptide linkers include, but are not limited to, GSG linkers (e.g., [GS]₄GG [SEQ ID NO: 347]), A(EAAAK)₃A (SEQ ID NO: 348), and Whitlow linkers (e.g., a “KEGS (SEQ ID NO: 446)” linker such as the amino acid sequence KESGSVSSEQLAQFRSLD (SEQ ID NO: 349), an eGK linker such as the amino acid sequence EGKSSGSGSESKST (SEQ ID NO: 350), an LR1 linker such as the amino acid sequence SGGGGSGGGGSGGGGSGGGGSGGGSLQ (SEQ ID NO: 215), and linkers described in more detail in Issued U.S. Pat. No. 5,990,275 herein incorporated by reference). Additional polypeptide linkers include SEQ ID NO: 194, SEQ ID NO: 196, and SEQ ID NO: 197. Other polypeptide linkers may be selected based on desired properties (e.g., length, flexibility, amino acid composition etc.) and are known to those skilled in the art.

In general, the cell-membrane tethering domain is oriented such that the secreted effector molecule and the protease cleavage site are extracellularly exposed following insertion into, or association with, the cell membrane, such that the protease cleavage site is capable of being cleaved by its respective protease and releasing (“secreting”) the effector molecule into the extracellular space.

### Degron Systems and Domains

In some embodiments, any of the proteins described herein can include a degron domain including, but not limited to, a cytokine, a CAR, a protease, a transcription factor, a promoter or constituent of a promoter system (e.g., an ACP), and/or any of the membrane-cleavable chimeric protein described herein. In general, the degron domain can be any amino acid sequence motif capable of directing regulated degradation, such as regulated degradation through a ubiquitin-mediated pathway. In the presence of an immunomodulatory drug (IMiD), the degron domain directs ubiquitin-mediated degradation of a degron-fusion protein.

The degron domain can be a cereblon (CRBN) polypeptide substrate domain capable of binding CRBN in response to an immunomodulatory drug (IMiD) including, but not limited to, IKZF1, IKZF3, CK1a, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN. The CRBN polypeptide substrate domain can be a chimeric fusion product of native CRBN polypeptide sequences, such as a IKZF3/ZFP91/IKZF3 chimeric fusion product having the amino acid sequence of FNVLMVHKRSHTGERPLQCEICGFTCRQKGNLLRHIKLHTGEKPFKCHLCNYACQRRD AL (SEQ ID NO: 175). Degron domains, and in particular CRBN degron systems, are described in more detail in International Application Pub. No. WO2019/089592A1, herein incorporated by reference for all purposes. Other examples of degron domains include, but are not limited to HCV NS4 degron, PEST (two copies of residues 277-307 of human IκBα; SEQ ID NO: 161), GRR (residues 352-408 of human p105; SEQ ID NO: 162), DRR (residues 210-295 of yeast Cdc34; SEQ ID NO: 163), SNS (tandem repeat of SP2 and NB (SP2-NB-SP2 of influenza A or influenza B; e.g., SEQ ID NO: 164), RPB (four copies of residues 1688-1702 of yeast RPB; SEQ ID NO: 165), SPmix (tandem repeat of SP1 and SP2 (SP2-SP1-SP2-SP1-SP2 of influenza A virus M2 protein; SEQ ID NO: 166), NS2 (three copies of residues 79-93 of influenza A virus NS protein; SEQ ID NO: 167), ODC (residues 106-142 of omithine decarboxylase; SEQ ID NO: 168), Nek2A, mouse ODC (residues 422-461; SEQ ID NO: 169), mouse ODC DA (residues 422-461 of mODC including D433A and D434A point mutations), an APC/C degron, a COP1E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron (SEQ ID NO: 345), an Siah binding motif, an SPOP SBC docking motif, or a PCNA binding PIP box.

Regulated degradation can be drug-inducible. Drugs capable of mediating/regulating degradation can be small-molecule compounds. Drugs capable of mediating/regulating degradation can include an “immunomodulatory drug” (IMiD). In general, as used herein, IMiDs refer to a class of small-molecule immunomodulatory drugs containing an imide group. Cereblon (CRBN) is known target of IMiDs and binding of an IMiD to CRBN or a CRBN polypeptide substrate domain alters the substrate specificity of the CRBN E3 ubiquitin ligase complex leading to degradation of proteins having a CRBN polypeptide substrate domain (e.g., any of secretable effector molecules or other proteins of interest described herein). For degron domains having a CRBN polypeptide substrate domain, examples of imide-containing IMiDs include, but are not limited to, a thalidomide, a lenalidomide, or a pomalidomide. The IMiD can be an FDA-approved drug.

Proteins described herein can contain a degron domain (e.g., referred to as “D” in the formula S-C-MT-D or D-MT-C-S for membrane-cleavable chimeric proteins described herein). In the absence of an IMiD, degron/ubiquitin-mediated degradation of the chimeric protein does not occur. Following expression and localization of the chimeric protein into the cell membrane, the protease cleavage site directs cleavage of the chimeric protein such that the effector molecule is released (“secreted”) into the extracellular space. In the presence of an immunomodulatory drug (IMiD), the degron domain directs ubiquitin-mediated degradation of the chimeric protein such that secretion of the effector molecule is reduced or eliminated. In general, for membrane-cleavable chimeric proteins fused to a degron domain, the degron domain is the terminal cytoplasmic-oriented domain, specifically relative to the cell membrane tethering domain, e.g., the most C-terminal domain in the formula S-C-MT-D or the most N-terminal domain in the formula D-MT-C-S. The degron domain can be connected to the cell membrane tethering domain by a polypeptide linker, i.e., a polypeptide sequence not generally considered to be part of the cell membrane tethering domain or the degron domain. A polypeptide linker can be any amino acid sequence that connects a first polypeptide sequence and a second polypeptide sequence. A polypeptide linker can be a flexible linker (e.g., a Gly-Ser-Gly sequence). Examples of polypeptide linkers include, but are not limited to, GSG linkers (e.g., [GS]₄GG [SEQ ID NO: 347]), A(EAAAK)₃A (SEQ ID NO: 348), and Whitlow linkers (e.g., a “KEGS (SEQ ID NO: 446)” linker such as the amino acid sequence KESGSVSSEQLAQFRSLD (SEQ ID NO: 349), an eGK linker such as the amino acid sequence EGKSSGSGSESKST (SEQ ID NO: 350), an LR1 linker such as the amino acid sequence SGGGGSGGGGSGGGGSGGGGSGGGSLQ (SEQ ID NO: 215), and linkers described in more detail in Issued U.S. Pat. No. 5,990,275 herein incorporated by reference). Additional polypeptide linkers include SEQ ID NO: 194, SEQ ID NO: 196, and SEQ ID NO: 197. Other polypeptide linkers may be selected based on desired properties (e.g., length, flexibility, amino acid composition etc.) and are known to those skilled in the art. In general, the degron is oriented in relation to the cell membrane tethering domain such that the degron is exposed to the cytosol following localization to the cell membrane such that the degron domain is capable of mediating degradation (e.g., exposure to the cytosol and cytosol) and is capable of mediating ubiquitin-mediated degradation.

For degron-fusion proteins, the degron domain can be N-terminal or C-terminal of the protein of interest, e.g., the effector molecule. The degron domain can be connected to the protein of interest by a polypeptide linker, i.e., a polypeptide sequence not generally considered to be part of the protein of interest or the degron domain. A polypeptide linker can be any amino acid sequence that connects a first polypeptide sequence and a second polypeptide sequence. A polypeptide linker can be a flexible linker (e.g., a Gly-Ser-Gly sequence). Examples of polypeptide linkers include, but are not limited to, GSG linkers (e.g., [GS]₄GG [SEQ ID NO: 347]), A(EAAAK)₃A (SEQ ID NO: 348), and Whitlow linkers (e.g., a “KEGS (SEQ ID NO: 446)” linker such as the amino acid sequence KESGSVSSEQLAQFRSLD (SEQ ID NO: 349), an eGK linker such as the amino acid sequence EGKSSGSGSESKST (SEQ ID NO: 350), an LR1 linker such as the amino acid sequence SGGGGSGGGGSGGGGSGGGGSGGGSLQ (SEQ ID NO: 215), and linkers described in more detail in Issued U.S. Pat. No. 5,990,275 herein incorporated by reference). Additional polypeptide linkers include SEQ ID NO: 194, SEQ ID NO: 196, and SEQ ID NO: 197. Other polypeptide linkers may be selected based on desired properties (e.g., length, flexibility, amino acid composition etc.) and are known to those skilled in the art. A polypeptide linker can be cleavable, e.g., any of the protease cleavage sites described herein.

### Engineered Nucleic Acids

Provided herein are engineered nucleic acids (e.g., an expression cassette) encoding at least one protein of the present disclosure, such as the cytokines, CARs, ACPs, and/or membrane-cleavable chimeric proteins having the formula S-C-MT or MT-C-S described herein. Provided herein are engineered nucleic acids (e.g., an expression cassette) encoding two or more proteins, such as two or more of the cytokines, CARs, ACPs, and/or membrane-cleavable chimeric proteins having the formula S-C-MT or MT-C-S described herein.

In certain embodiments described herein, the engineered nucleic acids encode an expression cassette containing a promoter and an exogenous polynucleotide sequence encoding the cytokines, CARs, ACPs, and/or membrane-cleavable chimeric protein, oriented from N-terminal to C-terminal, having the formula: S-C-MT or MT-C-S. S refers to a secretable effector molecule. C refers to a protease cleavage site. MT refers to a cell membrane tethering domain. The promoter is operably linked to the exogenous polynucleotide sequence and S-C-MT or MT-C-S is configured to be expressed as a single polypeptide.

In certain embodiments described herein, the engineered nucleic acids encode an expression cassette containing a promoter and an exogenous polynucleotide sequence encoding a cytokine. In certain embodiments described herein, the engineered nucleic acids encode an expression cassette containing a promoter and an exogenous polynucleotide sequence encoding a CAR. In certain embodiments described herein, the engineered nucleic acids encode an expression cassette containing a promoter and an exogenous polynucleotide sequence encoding a membrane-cleavable chimeric protein having a protein of interest (e.g., any of the effector molecules described herein). The promoter is operably linked to the exogenous polynucleotide sequence and the membrane-cleavable chimeric protein is configured to be expressed as a single polypeptide.

In certain embodiments described herein, the engineered nucleic acids encode an expression cassette containing a promoter and an exogenous polynucleotide sequence encoding a combination of the cytokines, CARs, ACPs, and/or membrane-cleavable chimeric proteins described herein. In certain embodiments described herein, the engineered nucleic acids encode an expression cassette containing a promoter and an exogenous polynucleotide sequence encoding a cytokine and CAR. In certain embodiments described herein, the engineered nucleic acids encode an expression cassette containing a promoter and an exogenous polynucleotide sequence encoding a cytokine and an ACP.

In certain embodiments described herein, the engineered nucleic acids encode two or more expression cassettes each containing a promoter and an exogenous polynucleotide sequence encoding a cytokine, CAR, ACP, and/or membrane-cleavable chimeric protein described herein. In certain embodiments described herein, the engineered nucleic acids encode two or more expression cassettes each containing a promoter and each separately encoding an exogenous polynucleotide sequence encoding a cytokine and CAR, respectively. In certain embodiments described herein, the engineered nucleic acids encode two or more expression cassettes each containing a promoter and each separately encoding an exogenous polynucleotide sequence encoding a cytokine and an ACP, respectively. In certain embodiments, the two or more expression cassettes are oriented in a head-to-tail orientation. In certain embodiments, the two or more expression cassettes are oriented in a head-to-head orientation. In certain embodiments, the two or more expression cassettes are oriented in a tail-to-tail orientation. In some cases, each expression cassette contains its own promoter to drive expression of the polynucleotide sequence encoding a cytokine and/or CAR. In certain embodiments, the cytokine and CAR are organized as such: 5′-cytokine-CAR-3′ or 5′-CAR-cytokine-3′.

An “engineered nucleic acid” is a nucleic acid that does not occur in nature. It should be understood, however, that while an engineered nucleic acid as a whole is not naturally- occurring, it may include nucleotide sequences that occur in nature. In some embodiments, an engineered nucleic acid comprises nucleotide sequences from different organisms (e.g., from different species). For example, in some embodiments, an engineered nucleic acid includes a murine nucleotide sequence, a bacterial nucleotide sequence, a human nucleotide sequence, and/or a viral nucleotide sequence. The term “engineered nucleic acids” includes recombinant nucleic acids and synthetic nucleic acids. A “recombinant nucleic acid” refers to a molecule that is constructed by joining nucleic acid molecules and, in some embodiments, can replicate in a live cell. A “synthetic nucleic acid” refers to a molecule that is amplified or chemically, or by other means, synthesized. Synthetic nucleic acids include those that are chemically modified, or otherwise modified, but can base pair with naturally- occurring nucleic acid molecules. Modifications include, but are not limited to, one or more modified internucleotide linkages and non-natural nucleic acids. Modifications are described in further detail in U.S. Pat. No. 6,673,611 and U.S. Application Publication 2004/0019001 and, each of which is incorporated by reference in their entirety. Modified internucleotide linkages can be a phosphorodithioate or phosphorothioate linkage. Non-natural nucleic acids can be a locked nucleic acid (LNA), a peptide nucleic acid (PNA), glycol nucleic acid (GNA), a phosphorodiamidate morpholino oligomer (PMO or “morpholino”), and threose nucleic acid (TNA). Non-natural nucleic acids are described in further detail in International Application WO 1998/039352, U.S. Application Pub. No. 2013/0156849, and U.S. Pat. Nos. 6,670,461; 5,539,082; 5,185,444, each herein incorporated by reference in their entirety. Recombinant nucleic acids and synthetic nucleic acids also include those molecules that result from the replication of either of the foregoing. Engineered nucleic acid of the present disclosure may be encoded by a single molecule (e.g., included in the same plasmid or other vector) or by multiple different molecules (e.g., multiple different independently-replicating molecules). Engineered nucleic acids can be an isolated nucleic acid. Isolated nucleic acids include, but are not limited to a cDNA polynucleotide, an RNA polynucleotide, an RNAi oligonucleotide (e.g., siRNAs, miRNAs, antisense oligonucleotides, shRNAs, etc.), an mRNA polynucleotide, a circular plasmid, a linear DNA fragment, a vector, a minicircle, a ssDNA, a bacterial artificial chromosome (BAC), and yeast artificial chromosome (YAC), and an oligonucleotide.

Engineered nucleic acid of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the Y extension activity of a DNA polymerase and DNA ligase activity. The 5′ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Clontech).

### Promoters

In general, in all embodiments described herein, the engineered nucleic acids encoding the proteins herein (e.g., a cytokine, CAR, ACP, and/or membrane-cleavable chimeric protein described herein) encode an expression cassette containing a promoter and an exogenous polynucleotide sequence encoding the protein. In some embodiments, an engineered nucleic acid (e.g., an engineered nucleic acid comprising an expression cassette) comprises a promoter operably linked to a nucleotide sequence (e.g., an exogenous polynucleotide sequence) encoding at least 2 distinct proteins. For example, the engineered nucleic acid may comprise a promoter operably linked to a nucleotide sequence encoding at least 3, at least 4, at least 5, at least 6, at least 7, at least 8, at least 8, at least 9, or at least 10 distinct proteins. In some embodiments, an engineered nucleic acid comprises a promoter operably linked to a nucleotide sequence encoding 1, 2, 3, 4, 5, 6, 7, 8, 9, 10, or more distinct proteins. In some embodiments, an engineered nucleic acid (e.g., an engineered nucleic acid comprising an expression cassette) comprises a promoter operably linked to a nucleotide sequence (e.g., an exogenous polynucleotide sequence) encoding at least 2 cytokines. For example, the engineered nucleic acid may comprise a promoter operably linked to a nucleotide sequence encoding at least 3, at least 4, at least 5, at least 6, at least 7, at least 8, at least 8, at least 9, or at least 10 cytokines. In some embodiments, an engineered nucleic acid comprises a promoter operably linked to a nucleotide sequence encoding 1, 2, 3, 4, 5, 6, 7, 8, 9, 10, or more cytokines. In some embodiments, an engineered nucleic acid (e.g., an engineered nucleic acid comprising an expression cassette) comprises a promoter operably linked to a nucleotide sequence (e.g., an exogenous polynucleotide sequence) encoding at least 2 membrane-cleavable chimeric proteins. For example, the engineered nucleic acid may comprise a promoter operably linked to a nucleotide sequence encoding at least 3, at least 4, at least 5, at least 6, at least 7, at least 8, at least 8, at least 9, or at least 10 membrane-cleavable chimeric proteins. In some embodiments, an engineered nucleic acid comprises a promoter operably linked to a nucleotide sequence encoding 1, 2, 3, 4, 5, 6, 7, 8, 9, 10, or more membrane-cleavable chimeric proteins.

A “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

Promoters of an engineered nucleic acid may be “inducible promoters,” which refer to promoters that are characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation a repressor that is preventing the promoter from driving transcription. Conversely, deactivation of transcription may involve directly acting on a promoter to prevent transcription or indirectly acting on a promoter by activating a repressor that then acts on the promoter.

A promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA-binding domain (DBD), an interferon-gamma-activated sequence (GAS) (Decker, T. et al. J Interferon Cytokine Res. 1997 March;17(3):121-34, incorporated herein by reference), an interferon-stimulated response element (ISRE) (Han, K. J. et al. J Biol Chem. 2004 Apr. 9; 279(15):15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271: 9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of responsive promoters (also referred to as “inducible promoters”) (e.g., TGF-beta responsive promoters) are listed in Table 5A, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Homer, M. & Weber, W. FEBS Letters 586 (2012) 20784-2096m, and references cited therein). Non-limiting examples of components of inducible promoters include those presented in Table 5B.

TABLE 5A
Examples of Responsive Promoters

| | | | | Response to | |
|---|---|---|---|---|---|
| | Promoter and | Transcription | | inducer | |
| System | operator | factor (TF) | Inducer molecule | TF | T |
| Transcriptional activator-responsive promoters | | | | | |
| AIR | PAIR (OalcA- | AlcR | Acetaldehyde | n.d. | A |
| | PhCMVmin) | | | | |
| ART | PART (OARG- | ArgR-VP16 | 1-Arginine | B | A |
| | PhCMVmin) | | | | |
| BIT | PBIT3 (OBirA3- | BIT (BirA- | Biotin | B | A |
| | PhCMVmin) | VP16) | | | |
| Cumate - activator | PCR5 (OCuO6- | cTA (CymR- | Cumate | D | DA |
| | PhCMVmin) | VP16) | | | |
| Cumate - reverse | PCR5 (OCuO6- | rcTA (rCymR- | Cumate | B | A |
| activator | PhCMVmin) | VP16) | | | |
| E-OFF | PETR (OETR- | ET (E-VP16) | Erythromycin | D | DA |
| | PhCMVmin) | | | | |
| NICE-OFF | PNIC (ONIC- | NT (HdnoR- | 6-Hydroxy-nicotine | D | DA |
| | PhCMVmin) | VP16) | | | |
| PEACE | PTtgR1 (OTtgR- | TtgAl (TtgR- | Phloretin | D | DA |
| | PhCMVmin) | VP16) | | | |
| PIP-OFF | PPIR (OPIR- | PIT (PIP- | Pristinamycin I | D | DA |
| | Phsp70min) | VP16) | | | |
| QuoRex | PSCA (OscbR- | SCA (ScbR- | SCB1 | D | DA |
| | PhCMVmin)PSPA | VP16) | | | |
| | (OpapRI- | | | | |
| | PhCMVmin) | | | | |
| Redox | PROP (OROP- | REDOX | NADH | D | DA |
| | PhCMVmin) | (REX-VP16) | | | |
| TET-OFF | PhCMV*-1 | tTA (TetR- | Tetracycline | D | DA |
| | (OtetO7- | VP16) | | | |
| | PhCMVmin) | | | | |
| TET-ON | PhCMV*-1 | rtTA (rTetR- | Doxycycline | B | A |
| | (OtetO7- | VP16) | | | |
| | PhCMVmin) | | | | |
| TIGR | PCTA (OrheO- | CTA (RheA- | Heat | D | DA |
| | PhCMVmin) | VP16) | | | |
| TraR | O7x(tra box)- | p65-TraR | 3-Oxo-C8-HSL | B | A |
| | PhCMVmin | | | | |
| VAC-OFF | P1VanO2 | VanAl (VanR- | Vanillic acid | D | DA |
| | (OVanO2- | VP16) | | | |
| | PhCMVmin) | | | | |
| Transcriptional repressor-responsive promoters | | | | | |
| Cumate - repressor | PCuO (PCMV5- | CymR | Cumate | D | DR |
| | OCuO) | | | | |
| E-ON | PETRON8 | E-KRAB | Erythromycin | D | DR |
| | (PSV40-OETR8) | | | | |
| NICE-ON | PNIC (PSV40- | NS (HdnoR | 6-Hydroxy-nicotine | D | DR |
| | ONIC8) | KRAB) | | | |
| PIP-ON | PPIRON (PSV40- | PIT3 (PIP- | Pristinamycin I | D | DR |
| | OPIR3) | KRAB | | | |
| Q-ON | PSCAON8 | SCS (ScbR- | SCB1 | D | DR |
| | (PSV40-OscbR8) | KRAB) | | | |
| TET-ON | OtetO-PHPRT | tTS-H4 (TetR- | Doxycycline | D | DR |
| repressor-based | | HDAC4) | | | |
| T-REX | PTetO (PhCMV- | TetR | Tetracycline | D | DR |
| | OtetO2) | | | | |
| UREX | PUREX8 (PSV40- | mUTS | Uric acid | D | DR |
| | OhucO8) | (KRAB-HucR) | | | |
| VAC-ON | PVanON8 | VanA4 (VanR- | Vanillic acid | D | DR |
| | (PhCMV- | KRAB) | | | |
| | OVanO8) | | | | |
| Hybrid promoters | | | | | |
| QuoRexPIP- | OscbR8-OPIR3- | SCAPIT3 | SCB1Pristinamycin I | DD | DADR |
| ON(NOT IF gate) | PhCMVmin | | | | |
| QuoRexE- | OscbR-OETR8- | SCAE-KRAB | SCB1Erythromycin | DD | DADR |
| ON(NOT IF gate) | PhCMVmin | | | | |
| TET-OFFE- | OtetO7-OETR8- | tTAE-KRAB | TetracyclineErythromycin | DD | DADR |
| ON(NOT IF gate) | PhCMVmin | | | | |
| TET-OFFPIP- | OtetO7-OPIR3- | tTAPIT3E- | TetracyclinePristinamycin | DDD | DADRDR |
| ONE-ON | OETR8- | KRAB | IErythromycin | | |
| | PhCMVmin | | | | |

TABLE 5B
Exemplary Components of Inducible Promoters

| Name | DNA SEQUENCE |
|---|---|
| minimal promoter; minP | AGAGGGTATATAATGGAAGCTCGACTTCCAG (SEQ ID NO: 1) |
| NFKB response element | GGGAATTTCCGGGGACTTTCCGGGAATTTCCGGGGACTTTCCGGGAAT |
| protein promoter; 5x | TTCC (SEQ ID NO: 2) |
| NFKB-RE | |
| CREB response element | CACCAGACAGTGACGTCAGCTGCCAGATCCCATGGCCGTCATACTGTG |
| protein promoter; 4x CRE | ACGTCTTTCAGACACCCCATTGACGTCAATGGGAGAA (SEQ ID NO: 3) |
| NFAT response element | GGAGGAAAAACTGTTTCATACAGAAGGCGTGGAGGAAAAACTGTTTC |
| protein promoter; 3x NFAT | ATACAGAAGGCGTGGAGGAAAAACTGTTTCATACAGAAGGCGT (SEQ |
| binding sites | ID NO: 4) |
| SRF response element | AGGATGTCCATATTAGGACATCTAGGATGTCCATATTAGGACATCTAG |
| protein promoter; 5x SRE | GATGTCCATATTAGGACATCTAGGATGTCCATATTAGGACATCTAGGA |
| | TGTCCATATTAGGACATCT (SEQ ID NO: 5) |
| SRF response element | AGTATGTCCATATTAGGACATCTACCATGTCCATATTAGGACATCTACT |
| protein promoter 2; 5x | ATGTCCATATTAGGACATCTTGTATGTCCATATTAGGACATCTAAAATG |
| SRF-RE | TCCATATTAGGACATCT (SEQ ID NO: 6) |
| AP1 response element | TGAGTCAGTGACTCAGTGAGTCAGTGACTCAGTGAGTCAGTGACTCAG |
| protein promoter; | (SEQ ID NO: 7) |
| 6x AP1-RE | |
| TCF-LEF response element | AGATCAAAGGGTTTAAGATCAAAGGGCTTAAGATCAAAGGGTATAAG |
| promoter; 8x TCF-LEF-RE | ATCAAAGGGCCTAAGATCAAAGGGACTAAGATCAAAGGGTTTAAGAT |
| | CAAAGGGCTTAAGATCAAAGGGCCTA (SEQ ID NO: 8) |
| SBEx4 | GTCTAGACGTCTAGACGTCTAGACGTCTAGAC (SEQ ID NO: 9) |
| SMAD2/3 - CAGACA x4 | CAGACACAGACACAGACACAGACA (SEQ ID NO: 10) |
| STAT3 binding site | Ggatccggtactcgagatctgcgatctaagtaagcttggcattccgg |
| | tactgttggtaaagccac (SEQ ID NO: 11) |
| minCMV | taggcgtgtacggtgggaggcctatataagcagagctcgtttagtga |
| | accgtcagatcgcctgga (SEQ ID NO: 170) |
| YB TATA | TCTAGAGGGTATATAATGGGGGCCA (SEQ ID NO: 171) |
| minTK | Ttcgcatattaaggtgacgcgtgtggcctcgaacaccgagcgaccct |
| | gcagcgacccgcttaa (SEQ ID NO: 172) |

Non-limiting examples of promoters include the cytomegalovirus (CMV) promoter, the elongation factor 1-alpha (EF1a) promoter, the elongation factor (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter (see Table 5C).

TABLE 5C
Exemplary Constitutive Promoters

| | |
|---|---|
| Name | DNA SEQUENCE |
| CMV | GTTGACATTGATTATTGACTAGTTATTAATAGTAATCAAT |
| | TACGGGGTCATTAGTTCATAGCCCATATATGGAGTTCCGCGTTAC |
| | ATAACTTACGGTAAATGGCCCGCCTGGCTGACCGCCCAACGACCC |
| | CCGCCCATTGACGTCAATAATGACGTATGTTCCCATAGTAACGCC |
| | AATAGGGACTTTCCATTGACGTCAATGGGTGGAGTATTTACGGTA |
| | AACTGCCCACTTGGCAGTACATCAAGTGTATCATATGCCAAGTAC |
| | GCCCCCTATTGACGTCAATGACGGTAAATGGCCCGCCTGGCATTA |
| | TGCCCAGTACATGACCTTATGGGACTTTCCTACTTGGCAGTACAT |
| | CTACGTATTAGTCATCGCTATTACCATGGTGATGCGGTTTTGGCA |
| | GTACATCAATGGGCGTGGATAGCGGTTTGACTCACGGGGATTTCC |
| | AAGTCTCCACCCCATTGACGTCAATGGGAGTTTGTTTTGGCACCA |
| | AAATCAACGGGACTTTCCAAAATGTCGTAACAACTCCGCCCCATT |
| | GACGCAAATGGGCGGTAGGCGTGTACGGTGGGAGGTCTATATAAG |
| | CAGAGCTC |
| | (SEQ ID NO: 12) |
| EF1a | GGCTCCGGTGCCCGTCAGTGGGCAGAGCGCACATCGCCCACAGTC |
| | CCCGAGAAGTTGGGGGGAGGGGTCGGCAATTGAACCGGTGCCTAG |
| | AGAAGGTGGCGCGGGGTAAACTGGGAAAGTGATGCCGTGTACTGG |
| | CTCCGCCTTTTTCCCGAGGGTGGGGGAGAACCGTATATAAGTGCA |
| | GTAGTCGCCGTGAACGTTCTTTTTCGCAACGGGTTTGCCGCCAGA |
| | ACACAGGTAAGTGCCGTGTGTGGTTCCCGCGGGCCTGGCCTCTTT |
| | ACGGGTTATGGCCCTTGCGTGCCTTGAATTACTTCCACCTGGCTG |
| | CAGTACGTGATTCTTGATCCCGAGCTTCGGGTTGGAAGTGGGTGG |
| | GAGAGTTCGAGGCCTTGCGCTTAAGGAGCCCCTTCGCCTCGTGCT |
| | TGAGTTGAGGCCTGGCCTGGGCGCTGGGGCCGCCGCGTGCGAATC |
| | TGGTGGCACCTTCGCGCCTGTCTCGCTGCTTTCGATAAGTCTCTA |
| | GCCATTTAAAATTTTTGATGACCTGCTGCGACGCTTTTTTTCTGG |
| | CAAGATAGTCTTGTAAATGCGGGCCAAGATCTGCACACTGGTATT |
| | TCGGTTTTTGGGGCCGCGGGCGGCGACGGGGCCCGTGCGTCCCAG |
| | CGCACATGTTCGGCGAGGCGGGGCCTGCGAGCGCGACCACCGAGA |
| | ATCGGACGGGGGTAGTCTCAAGCTGGCCGGCCTGCTCTGGTGCCT |
| | GTCCTCGCGCCGCCGTGTATCGCCCCGCCCCGGGCGGCAAGGCTG |
| | GCCCGGTCGGCACCAGTTGCGTGAGCGGAAAGATGGCCGCTTCCC |
| | GGTCCTGCTGCAGGGAGCTCAAAATGGAGGACGCGGCGCTCGGGA |
| | GAGCGGGCGGGTGAGTCACCCACACAAAGGAAAAGGGCCTTTCCG |
| | TCCTCAGCCGTCGCTTCATGTGACTCCACGGAGTACCGGGCGCCG |
| | TCCAGGCACCTCGATTAGTTCTCGAGCTTTTGGAGTACGTCGTCT |
| | TTAGGTTGGGGGGAGGGGTTTTATGCGATGGAGTTTCCCCACACT |
| | GAGTGGGTGGAGACTGAAGTTAGGCCAGCTTGGCACTTGATGTAA |
| | TTCTCCTTGGAATTTGCCCTTTTTGAGTTTGGATCTTGGTTCATT |
| | CTCAAGCCTCAGACAGTGGTTCAAAGTTTTTTTCTTCCATTTCAG |
| | GTGTCGTGA |
| | (SEQ ID NO: 13) |
| EFS | GGATCTGCGATCGCTCCGGTGCCCGTCAGTGGGCAGAGCGCACAT |
| | CGCCCACAGTCCCCGAGAAGTTGGGGGGAGGGGTCGGCAATTGAA |
| | CCGGTGCCTAGAGAAGGTGGCGCGGGGTAAACTGGGAAAGTGATG |
| | TCGTGTACTGGCTCCGCCTTTTTCCCGAGGGTGGGGGAGAACCGT |
| | ATATAAGTGCAGTAGTCGCCGTGAACGTTCTTTTTCGCAACGGGT |
| | TTGCCGCCAGAACACAGCTGAAGCTTCGAGGGGCTCGCATCTCTC |
| | CTTCACGCGCCCGCCGCCCTACCTGAGGCCGCCATCCACGCCGGT |
| | TGAGTCGCGTTCTGCCGCCTCCCGCCTGTGGTGCCTCCTGAACTG |
| | CGTCCGCCGTCTAGGTAAGTTTAAAGCTCAGGTCGAGACCGGGCC |
| | TTTGTCCGGCGCTCCCTTGGAGCCTACCTAGACTCAGCCGGCTCT |
| | CCACGCTTTGCCTGACCCTGCTTGCTCAACTCTACGTCTTTGTTT |
| | CGTTTTCTGTTCTGCGCCGTTACAGATCCAAGCTGTGACCGGCGC |
| | CTAC |
| | (SEQ ID NO: 14) |
| MND | TTTATTTAGTCTCCAGAAAAAGGGGGGAATGAAAGACCCCACCTG |
| | TAGGTTTGGCAAGCTAGGATCAAGGTTAGGAACAGAGAGACAGCA |
| | GAATATGGGCCAAACAGGATATCTGTGGTAAGCAGTTCCTGCCCC |
| | GGCTCAGGGCCAAGAACAGTTGGAACAGCAGAATATGGGCCAAAC |
| | AGGATATCTGTGGTAAGCAGTTCCTGCCCCGGCTCAGGGCCAAGA |
| | ACAGATGGTCCCCAGATGCGGTCCCGCCCTCAGCAGTTTCTAGAG |
| | AACCATCAGATGTTTCCAGGGTGCCCCAAGGACCTGAAATGACCC |
| | TGTGCCTTATTTGAACTAACCAATCAGTTCGCTTCTCGCTTCTGT |
| | TCGCGCGCTTCTGCTCCCCGAGCTCAATAAAAGAGCCCA |
| | (SEQ ID NO: 15) |
| PGK | GGGGTTGGGGTTGCGCCTTTTCCAAGGCAGCCCTGGGTTTGCGCA |
| | GGGACGCGGCTGCTCTGGGCGTGGTTCCGGGAAACGCAGCGGCGC |
| | CGACCCTGGGTCTCGCACATTCTTCACGTCCGTTCGCAGCGTCAC |
| | CCGGATCTTCGCCGCTACCCTTGTGGGCCCCCCGGCGACGCTTCC |
| | TGCTCCGCCCCTAAGTCGGGAAGGTTCCTTGCGGTTCGCGGCGTG |
| | CCGGACGTGACAAACGGAAGCCGCACGTCTCACTAGTACCCTCGC |
| | AGACGGACAGCGCCAGGGAGCAATGGCAGCGCGCCGACCGCGATG |
| | GGCTGTGGCCAATAGCGGCTGCTCAGCGGGGCGCGCCGAGAGCAG |
| | CGGCCGGGAAGGGGCGGTGCGGGAGGCGGGGTGTGGGGCGGTAGT |
| | GTGGGCCCTGTTCCTGCCCGCGCGGTGTTCCGCATTCTGCAAGCC |
| | TCCGGAGCGCACGTCGGCAGTCGGCTCCCTCGTTGACCGAATCAC |
| | CGACCTCTCTCCCCAG |
| | (SEQ ID NO: 16) |
| SFFV | GTAACGCCATTTTGCAAGGCATGGAAAAATACCAAACCAAGAATA |
| | GAGAAGTTCAGATCAAGGGCGGGTACATGAAAATAGCTAACGTTG |
| | GGCCAAACAGGATATCTGCGGTGAGCAGTTTCGGCCCCGGCCCGG |
| | GGCCAAGAACAGATGGTCACCGCAGTTTCGGCCCCGGCCCGAGGC |
| | CAAGAACAGATGGTCCCCAGATATGGCCCAACCCTCAGCAGTTTC |
| | TTAAGACCCATCAGATGTTTCCAGGCTCCCCCAAGGACCTGAAAT |
| | GACCCTGCGCCTTATTTGAATTAACCAATCAGCCTGCTTCTCGCT |
| | TCTGTTCGCGCGCTTCTGCTTCCCGAGCTCTATAAAAGAGCTCAC |
| | AACCCCTCACTCGGCGCGCCAGTCCTCCGACAGACTGAGTCGCCC |
| | GGG |
| | (SEQ ID NO: 17) |
| SV40 | CTGTGGAATGTGTGTCAGTTAGGGTGTGGAAAGTCCCCAGGCTCC |
| | CCAGCAGGCAGAAGTATGCAAAGCATGCATCTCAATTAGTCAGCA |
| | ACCAGGTGTGGAAAGTCCCCAGGCTCCCCAGCAGGCAGAAGTATG |
| | CAAAGCATGCATCTCAATTAGTCAGCAACCATAGTCCCGCCCCTA |
| | ACTCCGCCCATCCCGCCCCTAACTCCGCCCAGTTCCGCCCATTCT |
| | CCGCCCCATGGCTGACTAATTTTTTTTATTTATGCAGAGGCCGAG |
| | GCCGCCTCTGCCTCTGAGCTATTCCAGAAGTAGTGAGGAGGCTTT |
| | TTTGGAGGCCTAGGCTTTTGCAAAAAGCT |
| | (SEQ ID NO: 18) |
| SV40 alt | GTGTGTCAGTTAGGGTGTGGAAAGTCCCCAGGCTCCCCAGCAGGC |
| | AGAAGTATGCAAAGCATGCATCTCAATTAGTCAGCAACCAGGTGT |
| | GGAAAGTCCCCAGGCTCCCCAGCAGGCAGAAGTATGCAAAGCATG |
| | CATCTCAATTAGTCAGCAACCATAGTCCCGCCCCTAACTCCGCCC |
| | ATCCCGCCCCTAACTCCGCCCAGTTCCGCCCATTCTCCGCCCCAT |
| | GGCTGACTAATTTTTTTTATTTATGCAGAGGCCGAGGCCGCCTCT |
| | GCCTCTGAGCTATTCCAGAAGTAGTGAGGAGGCTTTTTTGGAGGC |
| | CTAGGCTTTTGCAAA |
| | (SEQ ID NO: 295) |
| UbC | GCGCCGGGTTTTGGCGCCTCCCGCGGGCGCCCCCCTCCTCACGGC |
| | GAGCGCTGCCACGTCAGACGAAGGGCGCAGGAGCGTTCCTGATCC |
| | TTCCGCCCGGACGCTCAGGACAGCGGCCCGCTGCTCATAAGACTC |
| | GGCCTTAGAACCCCAGTATCAGCAGAAGGACATTTTAGGACGGGA |
| | CTTGGGTGACTCTAGGGCACTGGTTTTCTTTCCAGAGAGCGGAAC |
| | AGGCGAGGAAAAGTAGTCCCTTCTCGGCGATTCTGCGGAGGGATC |
| | TCCGTGGGGCGGTGAACGCCGATGATTATATAAGGACGCGCCGGG |
| | TGTGGCACAGCTAGTTCCGTCGCAGCCGGGATTTGGGTCGCGGTT |
| | CTTGTTTGTGGATCGCTGTGATCGTCACTTGGTGAGTTGCGGGCT |
| | GCTGGGCTGGCCGGGGCTTTCGTGGCCGCCGGGCCGCTCGGTGGG |
| | ACGGAAGCGTGTGGAGAGACCGCCAAGGGCTGTAGTCTGGGTCCG |
| | CGAGCAAGGTTGCCCTGAACTGGGGGTTGGGGGGAGCGCACAAAA |
| | TGGCGGCTGTTCCCGAGTCTTGAATGGAAGACGCTTGTAAGGCGG |
| | GCTGTGAGGTCGTTGAAACAAGGTGGGGGGCATGGTGGGCGGCAA |
| | GAACCCAAGGTCTTGAGGCCTTCGCTAATGCGGGAAAGCTCTTAT |
| | TCGGGTGAGATGGGCTGGGGCACCATCTGGGGACCCTGACGTGAA |
| | GTTTGTCACTGACTGGAGAACTCGGGTTTGTCGTCTGGTTGCGGG |
| | GGCGGCAGTTATGCGGTGCCGTTGGGCAGTGCACCCGTACCTTTG |
| | GGAGCGCGCGCCTCGTCGTGTCGTGACGTCACCCGTTCTGTTGGC |
| | TTATAATGCAGGGTGGGGCCACCTGCCGGTAGGTGTGCGGTAGGC |
| | TTTTCTCCGTCGCAGGACGCAGGGTTCGGGCCTAGGGTAGGCTCT |
| | CCTGAATCGACAGGCGCCGGACCTCTGGTGAGGGGAGGGATAAGT |
| | GAGGCGTCAGTTTCTTTGGTCGGTTTTATGTACCTATCTTCTTAA |
| | GTAGCTGAAGCTCCGGTTTTGAACTATGCGCTCGGGGTTGGCGAG |
| | TGTGTTTTGTGAAGTTTTTTAGGCACCTTTTGAAATGTAATCATT |
| | TGGGTCAATATGTAATTTTCAGTGTTAGACTAGTAAAGCTTCTGC |
| | AGGTCGACTCTAGAAAATTGTCCGCTAAATTCTGGCCGTTTTTGG |
| | CTTTTTTGTTAGAC |
| | (SEQ ID NO: 19) |
| hEFlaV1 | GGCTCCGGTGCCCGTCAGTGGGCAGAGCGCACATCGCCCACAGTC |
| | CCCGAGAAGTTGGGGGGAGGGGTCGGCAATTGAACCGGTGCCTAG |
| | AGAAGGTGGCGCGGGGTAAACTGGGAAAGTGATGTCGTGTACTGG |
| | CTCCGCCTTTTTCCCGAGGGTGGGGGAGAACCGTATATAAGTGCA |
| | GTAGTCGCCGTGAACGTTCTTTTTCGCAACGGGTTTGCCGCCAGA |
| | ACACAGGTAAGTGCCGTGTGTGGTTCCCGCGGGCCTG |
| | GCCTCTTTACGGGTTATGGCCCTTGCGTGCCTTGAATTACTTCCA |
| | CCTGGCTGCAGTACGTGATTCTTGATCCCGAGCTTCGGGTTGGAA |
| | GTGGGTGGGAGAGTTCGAGGCCTTGCGCTTAAGGAGCCCCTTCGC |
| | CTCGTGCTTGAGTTGAGGCCTGGCCTGGGCGCTGGGGCCGCCGCG |
| | TGCGAATCTGGTGGCACCTTCGCGCCTGTCTCGCTGCTTTCGATA |
| | AGTCTCTAGCCATTTAAAATTTTTGATGACCTGCTGCGACGCTTT |
| | TTTTCTGGCAAGATAGTCTTGTAAATGCGGGCCAAGATCTGCACA |
| | CTGGTATTTCGGTTTTTGGGGCCGCGGGCGGCGACGGGGCCCGTG |
| | CGTCCCAGCGCACATGTTCGGCGAGGCGGGGCCTGCGAGCGCGGC |
| | CACCGAGAATCGGACGGGGGTAGTCTCAAGCTGGCCGGCCTGCTC |
| | TGGTGCCTGGTCTCGCGCCGCCGTGTATCGCCCCGCCCTGGGCGG |
| | CAAGGCTGGCCCGGTCGGCACCAGTTGCGTGAGCGGAAAGATGGC |
| | CGCTTCCCGGCCCTGCTGCAGGGAGCTCAAAATGGAGGACGCGGC |
| | GCTCGGGAGAGCGGGCGGGTGAGTCACCCACACAAAGGAAAAGGG |
| | CCTTTCCGTCCTCAGCCGTCGCTTCATGTGACTCCACGGAGTACC |
| | GGGCGCCGTCCAGGCACCTCGATTAGTTCTCGAGCTTTTGGAGTA |
| | CGTCGTCTTTAGGTTGGGGGGAGGGGTTTTATGCGATGGAGTTTC |
| | CCCACACTGAGTGGGTGGAGACTGAAGTTAGGCCAGCTTGGCACT |
| | TGATGTAATTCTCCTTGGAATTTGCCCTTTTTGAGTTTGGATCTT |
| | GGTTCATTCTCAAGCCTCAGACAGTGGTTCAAAGTTTTTTTCTTC |
| | CATTTCAGGTGTCGTGA |
| | (SEQ ID NO: 20) |
| hCAGG | ACTAGTTATTAATAGTAATCAATTACGGGGTCATTAGTTCATAGC |
| | CCATATATGGAGTTCCGCGTTACATAACTTACGGTAAATGGCCCG |
| | CCTGGCTGACCGCCCAACGACCCCCGCCCATTGACGTCAATAATG |
| | ACGTATGTTCCCATAGTAACGCCAATAGGGACTTTCCATTGACGT |
| | CAATGGGTGGAGTATTTACGGTAAACTGCCCACTTGGCAGTACAT |
| | CAAGTGTATCATATGCCAAGTACGCCCCCTATTGACGTCAATGAC |
| | GGTAAATGGCCCGCCTGGCATTATGCCCAGTACATGACCTTATGG |
| | GACTTTCCTACTTGGCAGTACATCTACGTATTAGTCATCGCTATT |
| | ACCATGGTCGAGGTGAGCCCCACGTTCTGCTTCACTCTCCCCATC |
| | TCCCCCCCCTCCCCACCCCCAATTTTGTATTTATTTATTTTTTAA |
| | TTATTTTGTGCAGCGATGGGGGCGGGGGGGGGGGGGGGGCGCGCG |
| | CCAGGCGGGGCGGGGCGGGGCGAGGGGCGGGGCGGGGCGAGGCGG |
| | AGAGGTGCGGCGGCAGCCAATCAGAGCGGCGCGCTCCGAAAGTTT |
| | CCTTTTATGGCGAGGCGGCGGCGGCGGCGGCCCTATAAAAAGCGA |
| | AGCGCGCGGCGGGCGGGGAGTCGCTGCGACGCTGCCTTCGCCCCG |
| | TGCCCCGCTCCGCCGCCGCCTCGCGCCGCCCGCCCCGGCTCTGAC |
| | TGACCGCGTTACTCCCACAGGTGAGCGGGCGGGACGGCCCTTCTC |
| | CTCCGGGCTGTAATTAGCGCTTGGTTTAATGACGGCTTGTTTCTT |
| | TTCTGTGGCTGCGTGAAAGCCTTGAGGGGCTCCGGGAGGGCCCTT |
| | TGTGCGGGGGGAGCGGCTCGGGGGGTGCGTGCGTGTGTGTGTGCG |
| | TGGGGAGCGCCGCGTGCGGCTCCGCGCTGCCCGGCGGCTGTGAGC |
| | GCTGCGGGCGCGGCGCGGGGCTTTGTGCGCTCCGCAGTGTGCGCG |
| | AGGGGAGCGCGGCCGGGGGCGGTGCCCCGCGGTGCGGGGGGGGCT |
| | GCGAGGGGAACAAAGGCTGCGTGCGGGGTGTGTGCGTGGGGGGGT |
| | GAGCAGGGGGTGTGGGCGCGTCGGTCGGGCTGCAACCCCCCCTGC |
| | ACCCCCCTCCCCGAGTTGCTGAGCACGGCCCGGCTTCGGGTGCGG |
| | GGCTCCGTACGGGGCGTGGCGCGGGGCTCGCCGTGCCGGGCGGGG |
| | GGTGGCGGCAGGTGGGGGTGCCGGGCGGGGCGGGGCCGCCTCGGG |
| | CCGGGGAGGGCTCGGGGGAGGGGCGCGGCGGCCCCCGGAGCGCCG |
| | GCGGCTGTCGAGGCGCGGCGAGCCGCAGCCATTGCCTTTTATGGT |
| | AATCGTGCGAGAGGGCGCAGGGACTTCCTTTGTCCCAAATCTGTG |
| | CGGAGCCGAAATCTGGGAGGCGCCGCCGCACCCCCTCTAGCGGGC |
| | GCGGGGCGAAGCGGTGCGGCGCCGGCAGGAAGGAAATGGGCGGGG |
| | AGGGCCTTCGTGCGTCGCCGCGCCGCCGTCCCCTTCTCCCTCTCC |
| | AGCCTCGGGGCTGTCCGCGGGGGGACGGCTGCCTTCGGGGGGGAC |
| | GGGGCAGGGCGGGGTTCGGCTTCTGGCGTGTGACCGGCGGCTCTA |
| | GAGCCTCTGCTAACCATGTTCATGCCTTCTTCTTTTTCCTACAGC |
| | TCCTGGGCAACGTGCTGGTTATTGTGCTGTCTCATCATTTTGGCA |
| | AAGAATTC |
| | (SEQ ID NO: 21) |
| hEFlaV2 | Gggcagagcgcacatcgcccacagtccccgagaagttggggggag |
| | gggtcggcaattgaaccggtgcctagagaaggtggcgcggggtaa |
| | actgggaaagtgatgtcgtgtactggctccgcctttttcccgagg |
| | ggggggagaaccgtatataagtgcagtagtcgccgtgaacgttct |
| | ttttcgcaacgggtttgccgccagaacacag |
| | (SEQ ID NO: 22) |
| hACTb | CCACTAGTTCCATGTCCTTATATGGACTCATCTTTGCCTATTGCG |
| | ACACACACTCAATGAACACCTACTACGCGCTGCAAAGAGCCCCGC |
| | AGGCCTGAGGTGCCCCCACCTCACCACTCTTCCTATTTTTGTGTA |
| | AAAATCCAGCTTCTTGTCACCACCTCCAAGGAGGGGGAGGAGGAG |
| | GAAGGCAGGTTCCTCTAGGCTGAGCCGAATGCCCCTCTGTGGTCC |
| | CACGCCACTGATCGCTGCATGCCCACCACCTGGGTACACACAGTC |
| | TGTGATTCCCGGAGCAGAACGGACCCTGCCCACCCGGTCTTGTGT |
| | GCTACTCAGTGGACAGACCCAAGGCAAGAAAGGGTGACAAGGACA |
| | GGGTCTTCCCAGGCTGGCTTTGAGTTCCTAGCACCGCCCCGCCCC |
| | CAATCCTCTGTGGCACATGGAGTCTTGGTCCCCAGAGTCCCCCAG |
| | CGGCCTCCAGATGGTCTGGGAGGGCAGTTCAGCTGTGGCTGCGCA |
| | TAGCAGACATACAACGGACGGTGGGCCCAGACCCAGGCTGTGTAG |
| | ACCCAGCCCCCCCGCCCCGCAGTGCCTAGGTCACCCACTAACGCC |
| | CCAGGCCTGGTCTTGGCTGGGCGTGACTGTTACCCTCAAAAGCAG |
| | GCAGCTCCAGGGTAAAAGGTGCCCTGCCCTGTAGAGCCCACCTTC |
| | CTTCCCAGGGCTGCGGCTGGGTAGGTTTGTAGCCTTCATCACGGG |
| | CCACCTCCAGCCACTGGACCGCTGGCCCCTGCCCTGTCCTGGGGA |
| | GTGTGGTCCTGCGACTTCTAAGTGGCCGCAAGCCACCTGACTCCC |
| | CCAACACCACACTCTACCTCTCAAGCCCAGGTCTCTCCCTAGTGA |
| | CCCACCCAGCACATTTAGCTAGCTGAGCCCCACAGCCAGAGGTCC |
| | TCAGGCCCTGCTTTCAGGGCAGTTGCTCTGAAGTCGGCAAGGGGG |
| | AGTGACTGCCTGGCCACTCCATGCCCTCCAAGAGCTCCTTCTGCA |
| | GGAGCGTACAGAACCCAGGGCCCTGGCACCCGTGCAGACCCTGGC |
| | CCACCCCACCTGGGCGCTCAGTGCCCAAGAGATGTCCACACCTAG |
| | GATGTCCCGCGGTGGGTGGGGGGCCCGAGAGACGGGCAGGCCGGG |
| | GGCAGGCCTGGCCATGCGGGGCCGAACCGGGCACTGCCCAGCGTG |
| | GGGCGCGGGGGCCACGGCGCGCGCCCCCAGCCCCCGGGCCCAGCA |
| | CCCCAAGGCGGCCAACGCCAAAACTCTCCCTCCTCCTCTTCCTCA |
| | ATCTCGCTCTCGCTCTTTTTTTTTTTCGCAAAAGGAGGGGAGAGG |
| | GGGTAAAAAAATGCTGCACTGTGCGGCGAAGCCGGTGAGTGAGCG |
| | GCGCGGGGCCAATCAGCGTGCGCCGTTCCGAAAGTTGCCTTTTAT |
| | GGCTCGAGCGGCCGCGGCGGCGCCCTATAAAACCCAGCGGCGCGA |
| | CGCGCCACCACCGCCGAGACCGCGTCCGCCCCGCGAGCACAGAGC |
| | CTCGCCTTTGCCGATCCGCCGCCCGTCCACACCCGCCGCCAGgta |
| | agcccggccagccgaccggggcaggcggctcacggcccggccgca |
| | ggcggccgcggccccttcgcccgtgcagagccgccgtctgggccg |
| | cagcggggggcgcatggggggggaaccggaccgccgtggggggcg |
| | cgggagaagcccctgggcctccggagatgggggacaccccacgcc |
| | agttcggaggcgcgaggccgcgctcgggaggcgcgctccgggggt |
| | gccgctctcggggcgggggcaaccggggggtctttgtctgagccg |
| | ggctcttgccaatggggatcgcagggtgggcgcggcggagccccc |
| | gccaggcccggtgggggctggggcgccattgcgcgtgcgcgctgg |
| | tcctttgggcgctaactgcgtgcgcgctgggaattggcgctaatt |
| | gcgcgtgcgcgctgggactcaaggcgctaactgcgcgtgcgttct |
| | ggggcccggggtgccgcggcctgggctggggcgaaggcgggctcg |
| | gccggaaggggggggtcgccgcggctcccgggcgcttgcgcgcac |
| | ttcctgcccgagccgctggccgcccgagggtgtggccgctgcgtg |
| | cgcgcgcgccgacccggcgctgtttgaaccgggcggaggcggggc |
| | tggcgcccggttgggagggggttggggcctggcttcctgccgcgc |
| | gccgcggggacgcctccgaccagtgtttgccttttatggtaataa |
| | cgcggccggcccggcttcctttgtccccaatctgggcgcgcgccg |
| | gcgccccctggcggcctaaggactcggcgcgccggaagtggccag |
| | gggggggcgacctcggctcacagcgcgcccggctat |
| | (SEQ ID NO: 23) |
| heIF4A1 | GTTGATTTCCTTCATCCCTGGCACACGTCCAGGCAGTGTCGAATC |
| | CATCTCTGCTACAGGGGAAAACAAATAACATTTGAGTCCAGTGGA |
| | GACCGGGAGCAGAAGTAAAGGGAAGTGATAACCCCCAGAGCCCGG |
| | AAGCCTCTGGAGGCTGAGACCTCGCCCCCCTTGCGTGATAGGGCC |
| | TACGGAGCCACATGACCAAGGCACTGTCGCCTCCGCACGTGTGAG |
| | AGTGCAGGGCCCCAAGATGGCTGCCAGGCCTCGAGGCCTGACTCT |
| | TCTATGTCACTTCCGTACCGGCGAGAAAGGCGGGCCCTCCAGCCA |
| | ATGAGGCTGCGGGGGGGGCCTTCACCTTGATAGGCACTCGAGTTA |
| | TCCAATGGTGCCTGCGGGCCGGAGCGACTAGGAACTAACGTCATG |
| | CCGAGTTGCTGAGCGCCGGCAGGCGGGGCCGGGGCGGCCAAACCA |
| | ATGCGATGGCCGGGGCGGAGTCGGGCGCTCTATAAGTTGTCGATA |
| | GGCGGGCACTCCGCCCTAGTTTCTAAGGACCATG |
| | (SEQ ID NO: 24) |
| hGAPDH | AGTTCCCCAACTTTCCCGCCTCTCAGCCTTTGAAAGAAAGAAAGG |
| | GGAGGGGGCAGGCCGCGTGCAGTCGCGAGCGGTGCTGGGCTCCGG |
| | CTCCAATTCCCCATCTCAGTCGCTCCCAAAGTCCTTCTGTTTCAT |
| | CCAAGCGTGTAAGGGTCCCCGTCCTTGACTCCCTAGTGTCCTGCT |
| | GCCCACAGTCCAGTCCTGGGAACCAGCACCGATCACCTCCCATCG |
| | GGCCAATCTCAGTCCCTTCCCCCCTACGTCGGGGCCCACACGCTC |
| | GGTGCGTGCCCAGTTGAACCAGGCGGCTGCGGAAAAAAAAAAGCG |
| | GGGAGAAAGTAGGGCCCGGCTACTAGCGGTTTTACGGGCGCACGT |
| | AGCTCAGGCCTCAAGACCTTGGGCTGGGACTGGCTGAGCCTGGCG |
| | GGAGGCGGGGTCCGAGTCACCGCCTGCCGCCGCGCCCCCGGTTTC |
| | TATAAATTGAGCCCGCAGCCTCCCGCTTCGCTCTCTGCTCCTCCT |
| | GTTCGACAGTCAGCCGCATCTTCTTTTGCGTCGCCAGgtgaagac |
| | gggcggagagaaacccgggaggctagggacggcctgaaggcggca |
| | ggggcgggcgcaggccggatgtgttcgcgccgctgcggggtgggc |
| | ccgggcggcctccgcattgcaggggcgggcggaggacgtgatgcg |
| | gcgcgggctgggcatggaggcctggtgggggaggggaggggaggc |
| | gtgggtgtcggccggggccactaggcgctcactgttctctccctc |
| | cgcgcagCCGAGCCACATCGCTGAGACAC |
| | (SEQ ID NO: 25) |
| hGRP78 | AGTGCGGTTACCAGCGGAAATGCCTCGGGGTCAGAAGTCGCAGGA |
| | GAGATAGACAGCTGCTGAACCAATGGGACCAGCGGATGGGGCGGA |
| | TGTTATCTACCATTGGTGAACGTTAGAAACGAATAGCAGCCAATG |
| | AATCAGCTGGGGGGGCGGAGCAGTGACGTTTATTGCGGAGGGGGC |
| | CGCTTCGAATCGGCGGCGGCCAGCTTGGTGGCCTGGGCCAATGAA |
| | CGGCCTCCAACGAGCAGGGCCTTCACCAATCGGCGGCCTCCACGA |
| | CGGGGCTGGGGGAGGGTATATAAGCCGAGTAGGCGACGGTGAGGT |
| | CGACGCCGGCCAAGACAGCACAGACAGATTGACCTATTGGGGTGT |
| | TTCGCGAGTGTGAGAGGGAAGCGCCGCGGCCTGTATTTCTAGACC |
| | TGCCCTTCGCCTGGTTCGTGGCGCCTTGTGACCCCGGGCCCCTGC |
| | CGCCTGCAAGTCGGAAATTGCGCTGTGCTCCTGTGCTACGGCCTG |
| | TGGCTGGACTGCCTGCTGCTGCCCAACTGGCTGGCAC |
| | (SEQ ID NO: 26) |
| hGRP94 | TAGTTTCATCACCACCGCCACCCCCCCGCCCCCCCGCCATCTGAA |
| | AGGGTTCTAGGGGATTTGCAACCTCTCTCGTGTGTTTCTTCTTTC |
| | CGAGAAGCGCCGCCACACGAGAAAGCTGGCCGCGAAAGTCGTGCT |
| | GGAATCACTTCCAACGAAACCCCAGGCATAGATGGGAAAGGGTGA |
| | AGAACACGTTGCCATGGCTACCGTTTCCCCGGTCACGGAATAAAC |
| | GCTCTCTAGGATCCGGAAGTAGTTCCGCCGCGACCTCTCTAAAAG |
| | GATGGATGTGTTCTCTGCTTACATTCATTGGACGTTTTCCCTTAG |
| | AGGCCAAGGCCGCCCAGGCAAAGGGGCGGTCCCACGCGTGAGGGG |
| | CCCGCGGAGCCATTTGATTGGAGAAAAGCTGCAAACCCTGACCAA |
| | TCGGAAGGAGCCACGCTTCGGGCATCGGTCACCGCACCTGGACAG |
| | CTCCGATTGGTGGACTTCCGCCCCCCCTCACGAATCCTCATTGGG |
| | TGCCGTGGGTGCGTGGTGCGGCGCGATTGGTGGGTTCATGTTTCC |
| | CGTCCCCCGCCCGCGAGAAGTGGGGGTGAAAAGCGGCCCGACCTG |
| | CTTGGGGTGTAGTGGGCGGACCGCGCGGCTGGAGGTGTGAGGATC |
| | CGAACCCAGGGGGGGGGGGGAGGCGGCTCCTGCGATCGAAGGGGA |
| | CTTGAGACTCACCGGCCGCACGTC |
| | (SEQ ID NO: 27) |
| hHSP70 | GGGCCGCCCACTCCCCCTTCCTCTCAGGGTCCCTGTCCCCTCCAG |
| | TGAATCCCAGAAGACTCTGGAGAGTTCTGAGCAGGGGGCGGCACT |
| | CTGGCCTCTGATTGGTCCAAGGAAGGCTGGGGGGCAGGACGGGAG |
| | GCGAAAACCCTGGAATATTCCCGACCTGGCAGCCTCATCGAGCTC |
| | GGTGATTGGCTCAGAAGGGAAAAGGCGGGTCTCCGTGACGACTTA |
| | TAAAAGCCCAGGGGCAAGCGGTCCGGATAACGGCTAGCCTGAGGA |
| | GCTGCTGCGACAGTCCACTACCTTTTTCGAGAGTGACTCCCGTTG |
| | TCCCAAGGCTTCCCAGAGCGAACCTGTGCGGCTGCAGGCACCGGC |
| | GCGTCGAGTTTCCGGCGTCCGGAAGGACCGAGCTCTTCTCGCGGA |
| | TCCAGTGTTCCGTTTCCAGCCCCCAATCTCAGAGCGGAGCCGACA |
| | GAGAGCAGGGAACCC |
| | (SEQ IDNO: 28) |
| hKINb | GCCCCACCCCCGTCCGCGTTACAACCGGGAGGCCCGCTGGGTCCT |
| | GCACCGTCACCCTCCTCCCTGTGACCGCCCACCTGATACCCAAAC |
| | AACTTTCTCGCCCCTCCAGTCCCCAGCTCGCCGAGCGCTTGCGGG |
| | GAGCCACCCAGCCTCAGTTTCCCCAGCCCCGGGCGGGGCGAGGGG |
| | CGATGACGTCATGCCGGCGCGCGGCATTGTGGGGCGGGGCGAGGC |
| | GGGGCGCCGGGGGGAGCAACACTGAGACGCCATTTTCGGCGGCGG |
| | GAGCGGCGCAGGCGGCCGAGCGGGACTGGCTGGGTCGGCTGGGCT |
| | GCTGGTGCGAGGAGCCGCGGGGCTGTGCTCGGCGGCCAAGGGGAC |
| | AGCGCGTGGGTGGCCGAGGATGCTGCGGGGCGGTAGCTCCGGCGC |
| | CCCTCGCTGGTGACTGCTGCGCCGTGCCTCACACAGCCGAGGCGG |
| | GCTCGGCGCACAGTCGCTGCTCCGCGCTCGCGCCCGGCGGCGCTC |
| | CAGGTGCTGACAGCGCGAGAGAGCGCGGCCTCAGGAGCAACAC |
| | (SEQ ID NO: 29) |
| hUBIb | TTCCAGAGCTTTCGAGGAAGGTTTCTTCAACTCAAATTCATCCGC |
| | CTGATAATTTTCTTATATTTTCCTAAAGAAGGAAGAGAAGCGCAT |
| | AGAGGAGAAGGGAAATAATTTTTTAGGAGCCTTTCTTACGGCTAT |
| | GAGGAATTTGGGGCTCAGTTGAAAAGCCTAAACTGCCTCTCGGGA |
| | GGTTGGGCGCGGCGAACTACTTTCAGCGGCGCACGGAGACGGCGT |
| | CTACGTGAGGGGTGATAAGTGACGCAACACTCGTTGCATAAATTT |
| | GCGCTCCGCCAGCCCGGAGCATTTAGGGGCGGTTGGCTTTGTTGG |
| | GTGAGCTTGTTTGTGTCCCTGTGGGTGGACGTGGTTGGTGATTGG |
| | CAGGATCCTGGTATCCGCTAACAGgtactggcccacagccgtaaa |
| | gacctgcgggggcgtgagaggggggaatgggtgaggtcaagctgg |
| | aggcttcttggggttgggtgggccgctgaggggaggggagggcga |
| | ggtgacgcgacacccggcctttctgggagagtgggccttgttgac |
| | ctaaggggggcgagggcagttggcacgcgcacgcgccgacagaaa |
| | ctaacagacattaaccaacagcgattccgtcgcgtttacttggga |
| | ggaaggcggaaaagaggtagtttgtgtggcttctggaaaccctaa |
| | atttggaatcccagtatgagaatggtgtcccttcttgtgtttcaa |
| | tgggatttttacttcgcgagtcttgtgggtttggttttgttttca |
| | gtttgcctaacaccgtgcttaggtttgaggcagattggagttcgg |
| | tcgggggagtttgaatatccggaacagttagtggggaaagctgtg |
| | gacgcttggtaagagagcgctctggattttccgctgttgacgttg |
| | aaaccttgaatgacgaatttcgtattaagtgacttagccttgtaa |
| | aattgaggggaggcttgcggaatattaacgtatttaaggcatttt |
| | gaaggaatagttgctaattttgaagaatattaggtgtaaaagcaa |
| | gaaatacaatgatcctgaggtgacacgcttatgttttacttttaa |
| | actagGTCACC |
| | (SEQ ID NO: 30) |
| CAG | gacattgattattgactagttattaatagtaatcaattacggggt |
| | cattagttcatagcccatatatggagttccgcgttacataactta |
| | cggtaaatggcccgcctggctgaccgcccaacgacccccgcccat |
| | tgacgtcaataatgacgtatgttcccatagtaacgccaataggga |
| | ctttccattgacgtcaatgggtggagtatttacggtaaactgccc |
| | acttggcagtacatcaagtgtatcatatgccaagtacgcccccta |
| | ttgacgtcaatgacggtaaatggcccgcctggcattatgcccagt |
| | acatgaccttatgggactttcctacttggcagtacatctacgtat |
| | tagtcatcgctattaccatggtcgaggtgagccccacgttctgct |
| | tcactctccccatctcccccccctccccacccccaattttgtatt |
| | tatttattttttaattattttgtgcagcgatgggggcgggggggg |
| | ggggggggcgcgcgccaggcggggggggggggcgagggggggggg |
| | ggcgaggcggagaggtgcggcggcagccaatcagagcggcgcgct |
| | ccgaaagtttccttttatggcgaggggggggggggccctataaaa |
| | agcgaagcgcgcggcgggcg |
| | (SEQ ID NO: 173) |
| HLP | Tgtttgctgcttgcaatgtttgcccattttagggtggacacagga |
| | cgctgtggtttctgagccagggggcgactcagatcccagccagtg |
| | gacttagcccctgtttgctcctccgataactggggtgaccttggt |
| | taatattcaccagcagcctcccccgttgcccctctggatccactg |
| | cttaaatacggacgaggacagggccctgtctcctcagcttcaggc |
| | accaccactgacctgggacagtgaat |
| | (SEQ ID NO: 174) |

The promoter can be a tissue-specific promoter. In general, a tissue-specific promoter directs transcription of a nucleic acid, (e.g., the engineered nucleic acids encoding the proteins herein (e.g., a cytokine, CAR, ACP, and/or membrane-cleavable chimeric protein described herein) such that expression is limited to a specific cell type, organelle, or tissue. Tissue-specific promoters include, but are not limited to, albumin (liver specific, Pinkert et al., (1987)), lymphoid specific promoters (Calame and Eaton, 1988), particular promoters of T-cell receptors (Winoto and Baltimore, (1989)) and immunoglobulins; Banerji et al., (1983); Queen and Baltimore, 1983), neuron specific promoters (e.g. the neurofilament promoter; Byrne and Ruddle, 1989), pancreas specific promoters (Edlund et al., (1985)) or mammary gland specific promoters (milk whey promoter, U.S. Pat. No. 4,873,316 and European Application Publication No. 264,166) as well as developmentally regulated promoters such as the murine hox promoters (Kessel and Gruss, Science 249:374-379 (1990)) or the α-fetoprotein promoter (Campes and Tilghman, Genes Dev. 3:537-546 (1989)), the contents of each of which are fully incorporated by reference herein. The promoter can be constitutive in the respective specific cell type, organelle, or tissue. Tissue-specific promoters and/or regulatory elements can also include promoters from the liver fatty acid binding (FAB) protein gene, specific for colon epithelial cells; the insulin gene, specific for pancreatic cells; the transphyretin, α1-antitrypsin, plasminogen activator inhibitor type 1 (PAI-I), apolipoprotein A1 and LDL receptor genes, specific for liver cells; the myelin basic protein (MBP) gene, specific for oligodendrocytes; the glial fibrillary acidic protein (GFAP) gene, specific for glial cells; OPSIN, specific for targeting to the eye; and the neural-specific enolase (NSE) promoter that is specific for nerve cells. Examples of tissue-specific promoters include, but are not limited to, the promoter for creatine kinase, which has been used to direct expression in muscle and cardiac tissue and immunoglobulin heavy or light chain promoters for expression in B cells. Other tissue specific promoters include the human smooth muscle alpha-actin promoter. Exemplary tissue-specific expression elements for the liver include but are not limited to HMG-COA reductase promoter, sterol regulatory element 1, phosphoenol pyruvate carboxy kinase (PEPCK) promoter, human C-reactive protein (CRP) promoter, human glucokinase promoter, cholesterol L 7-alpha hydroylase (CYP-7) promoter, beta- galactosidase alpha-2,6 sialylkansferase promoter, insulin-like growth factor binding protein (IGFBP-I) promoter, aldolase B promoter, human transferrin promoter, and collagen type I promoter. Exemplary tissue-specific expression elements for the prostate include but are not limited to the prostatic acid phosphatase (PAP) promoter, prostatic secretory protein of 94 (PSP 94) promoter, prostate specific antigen complex promoter, and human glandular kallikrein gene promoter (hgt-1). Exemplary tissue- specific expression elements for gastric tissue include but are not limited to the human H+/K+-ATPase alpha subunit promoter. Exemplary tissue-specific expression elements for the pancreas include but are not limited to pancreatitis associated protein promoter (PAP), elastase 1 transcriptional enhancer, pancreas specific amylase and elastase enhancer promoter, and pancreatic cholesterol esterase gene promoter. Exemplary tissue-specific expression elements for the endometrium include, but are not limited to, the uteroglobin promoter. Exemplary tissue-specific expression elements for adrenal cells include, but are not limited to, cholesterol side-chain cleavage (SCC) promoter. Exemplary tissue-specific expression elements for the general nervous system include, but are not limited to, gamma-gamma enolase (neuron- specific enolase, NSE) promoter. Exemplary tissue-specific expression elements for the brain include, but are not limited to, the neurofilament heavy chain (NF-H) promoter. Exemplary tissue-specific expression elements for lymphocytes include, but are not limited to, the human CGL-1/granzyme B promoter, the terminal deoxy transferase (TdT), lambda 5, VpreB, and lck (lymphocyte specific tyrosine protein kinase p561ck) promoter, the humans CD2 promoter and its 3′transcriptional enhancer, and the human NK and T cell specific activation (NKG5) promoter. Exemplary tissue-specific expression elements for the colon include, but are not limited to, pp60c-src tyrosine kinase promoter, organ-specific neoantigens (OSNs) promoter, and colon specific antigen-P promoter. Tissue-specific expression elements for breast cells are for example, but are not limited to, the human alpha-lactalbumin promoter. Exemplary tissue-specific expression elements for the lung include, but are not limited to, the cystic fibrosis transmembrane conductance regulator (CFTR) gene promoter.

In some embodiments, a promoter of the present disclosure is modulated by signals within a tumor microenvironment. A tumor microenvironment is considered to modulate a promoter if, in the presence of the tumor microenvironment, the activity of the promoter is increased or decreased by at least 10%, relative to activity of the promoter in the absence of the tumor microenvironment. In some embodiments, the activity of the promoter is increased or decreased by at least 20%, at least 30%, at least 40%, at least 50%, at least 60%, at least 70%, at least 80%, at least 90%, at least 100%, relative to activity of the promoter in the absence of the tumor microenvironment. For example, the activity of the promoter is increased or decreased by 10-20%, 10-30%, 10-40%, 10-50%, 10-60%, 10-70%, 10-80%, 10-90%, 10-100%, 10-200%, 20-30%, 20-40%, 20-50%, 20-60%, 20-70%, 20-80%, 20-90%, 20-100%, 20-200%, 50-60%, 50-70%, 50-80%, 50-90%, 50-100%, or 50-200%, relative to activity of the promoter in the absence of the tumor microenvironment.

In some embodiments, the activity of the promoter is increased or decreased by at least 2 fold (e.g., 2, 3, 4, 5, 10, 25, 20, 25, 50, or 100 fold), relative to activity of the promoter in the absence of the tumor microenvironment. For example, the activity of the promoter is increased or decreased by at least 3 fold, at least 5 fold, at least 10 fold, at least 20 fold, at least 50 fold, or at least 100 fold, relative to activity of the promoter in the absence of the tumor microenvironment. In some embodiments, the activity of the promoter is increased or decreased by 2-10, 2-20, 2-30, 2-40, 2-50, 2-60, 2-70, 2-80, 2-90, or 2-100 fold, relative to activity of the promoter in the absence of the tumor microenvironment.

In some embodiments, a promoter of the present disclosure is activated under a hypoxic condition. A “hypoxic condition” is a condition where the body or a region of the body is deprived of adequate oxygen supply at the tissue level. Hypoxic conditions can cause inflammation (e.g., the level of inflammatory cytokines increase under hypoxic conditions). In some embodiments, the promoter that is activated under hypoxic condition is operably linked to a nucleotide encoding a protein that decreases the expression of activity of inflammatory cytokines, thus reducing the inflammation caused by the hypoxic condition. In some embodiments, the promoter that is activated under hypoxic conditions comprises a hypoxia responsive element (HRE). A “hypoxia responsive element (HRE)” is a response element that responds to hypoxia-inducible factor (HIF). The HRE, in some embodiments, comprises a consensus motif NCGTG (where N is either A or G).

### Activation-Conditional Control Polypeptide (ACP) Promoter Systems

In some embodiments, a synthetic promoter is a promoter system including an activation-conditional control polypeptide- (ACP-) binding domain sequence and a promoter sequence. Such a system is also referred to herein as an “ACP-responsive promoter.” In general, an ACP promoter system includes a first expression cassette encoding an activation-conditional control polypeptide (ACP) and a second expression cassette encoding an ACP-responsive promoter operably linked to an exogenous polynucleotide sequence, such as the exogenous polynucleotide sequence encoding the cytokines, including membrane-cleavable chimeric proteins versions of cytokines, described herein or any other protein of interest (e.g., a protease or CAR). In some embodiments, the first expression cassette and second expression cassette are each encoded by a separate engineered nucleic acid. In other embodiments, the first expression cassette and the second expression cassette are encoded by the same engineered nucleic acid. The ACP-responsive promoter can be operably linked to a nucleotide sequence encoding a single protein of interest or multiple proteins of interest. In some embodiments, a synthetic promoter comprises the nucleic acid sequence of AATTAACGGGTTTCGTAACAATCGCATGAGGATTCGCAACGCCTTTGAAGCAGTCG ACGCCGAAGTCCCGTCTCAGTAAAGGTTGAAGCAGTCGACGCCGAAGAATCGGACT GCCTTCGTATGAAGCAGTCGACGCCGAAGGTATCAGTCGCCTCGGAATGAAGCAGT CGACGCCGAAGATTCGTAAGAGGCTCACTCTCCCTTACACGGAGTGGATAACTAGT TCTAGAGGGTATATAATGGGGGCCAACGCGTACCGGTGTC (SEQ ID NO: 298). In some embodiments, a synthetic promoter comprises a sequence that is at least 90%, at least 91%, at least 92%, at least 93%, at least 94%, at least 95%, at least 96%, at least 97%, at least 98%, or at least 99% identical to SEQ ID NO: 298. In some embodiments, a synthetic promoter comprises the nucleic acid sequence of CGGGTTTCGTAACAATCGCATGAGGATTCGCAACGCCTTCGGCGTAGCCGATGTCG CGCTCCCGTCTCAGTAAAGGTCGGCGTAGCCGATGTCGCGCAATCGGACTGCCTTCG TACGGCGTAGCCGATGTCGCGCGTATCAGTCGCCTCGGAACGGCGTAGCCGATGTC GCGCATTCGTAAGAGGCTCACTCTCCCTTACACGGAGTGGATAACTAGTTCTAGAG GGTATATAATGGGGGCCA (SEQ ID NO: 299). In some embodiments, a synthetic promoter comprises a sequence that is at least 90%, at least 91%, at least 92%, at least 93%, at least 94%, at least 95%, at least 96%, at least 97%, at least 98%, or at least 99% identical to SEQ ID NO: 299.

The promoters of the ACP promoter system, e.g., either a promoter driving expression of the ACP or the promoter sequence of the ACP-responsive promoter, can include any of the promoter sequences described herein (see “Promoters” above). The ACP-responsive promoter can be derived from minP, NFkB response element, CREB response element, NFAT response element, SRF response element 1, SRF response element 2, AP1 response element, TCF-LEF response element promoter fusion, Hypoxia responsive element, SMAD binding element, STAT3 binding site, minCMV, YB_TATA, minTK, inducer molecule responsive promoters, and tandem repeats thereof. In some embodiments, the ACP-responsive promoter includes a minimal promoter.

In some embodiments, the ACP-binding domain includes one or more zinc finger binding sites. In some embodiments, the ACP-responsive promoter includes a minimal promoter and the ACP-binding domain includes one or more zinc finger binding sites. The ACP-binding domain can include 1, 2, 3, 4, 5,6 7, 8, 9, 10, or more zinc finger binding sites. In some embodiments, the transcription factor is a zinc-finger-containing transcription factor. In some embodiments, the zinc-finger-containing transcription factor is a synthetic transcription factor. In some embodiments, the ACP-binding domain includes one or more zinc finger binding sites and the ACP has a DNA-binding zinc finger protein domain (ZF protein domain). In some embodiments, the ACP has a DNA-binding zinc finger protein domain (ZF protein domain) and an effector domain. In some embodiments, the ACP-binding domain includes one or more zinc finger binding sites and the ACP has a DNA-binding zinc finger protein domain (ZF protein domain) and an effector domain. In some embodiments, the ZF protein domain is modular in design and is composed of zinc finger arrays (ZFA). A zinc finger array comprises multiple zinc finger protein motifs that are linked together. Each zinc finger motif binds to a different nucleic acid motif. This results in a ZFA with specificity to any desired nucleic acid sequence, e.g., a ZFA with desired specificity to an ACP-binding domain having a specific zinc finger binding site composition and/or configuration. The ZF motifs can be directly adjacent to each other, or separated by a flexible linker sequence. In some embodiments, a ZFA is an array, string, or chain of ZF motifs arranged in tandem. A ZFA can have 1, 2, 3, 4, 5, 6, 7, 8, 9, 10, 11, 12,1 3, 14, or 15 zinc finger motifs. The ZFA can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 zinc finger motifs. The ZF protein domain can have 1, 2, 3, 4, 5, 6, 7, 8, 9, 10, 11, 12, 13, 14, 15, or more ZFAs. The ZF domain can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 ZFAs. In some embodiments, the ZF protein domain comprises one to ten ZFA(s). In some embodiments, the ZF protein domain comprises at least one ZFA. In some embodiments, the ZF protein domain comprises at least two ZFAs. In some embodiments, the ZF protein domain comprises at least three ZFAs. In some embodiments, the ZF protein domain comprises at least four ZFAs. In some embodiments, the ZF protein domain comprises at least five ZFAs. In some embodiments, the ZF protein domain comprises at least ten ZFAs.

In some embodiments, the DNA-binding domain comprises a tetracycline (or derivative thereof) repressor (TetR) domain.

The ACP can also further include an effector domain, such as a transcriptional effector domain. For instance, a transcriptional effector domain can be the effector or activator domain of a transcription factor. Transcription factor activation domains are also known as transactivation domains, and act as scaffold domains for proteins such as transcription coregulators that act to activate or repress transcription of genes. Any suitable transcriptional effector domains can be used in the ACP including, but not limited to, a Herpes Simplex Virus Protein 16 (VP16) activation domain; an activation domain consisting of four tandem copies of VP16, a VP64 activation domain; a p65 activation domain of NFκB; an Epstein-Barr virus R transactivator (Rta) activation domain; a tripartite activator comprising the VP64, the p65, and the Rta activation domains, the tripartite activator is known as a VPR activation domain; a histone acetyltransferase (HAT) core domain of the human ETA-associated protein p300, known as a p300 HAT core activation domain; a Kroppel associated box (KRAB) repression domain; a Repressor Element Silencing Transcription Factor (REST) repression domain; a WRPW motif (SEQ ID NO: 346) of the hairy-related basic helix-loop-helix repressor proteins, the motif is known as a WRPW repression domain (SEQ ID NO: 346); a DNA (cytosine-5)-methyltransferase 3B (DNMT3B) repression domain; and an HP1 alpha chromoshadow repression domain, or any combination thereof.

In some embodiments, the effector domain is s transcription effector domain selected from: a Herpes Simplex Virus Protein 16 (VP16) activation domain; an activation domain consisting of four tandem copies of VP16, a VP64 activation domain; a p65 activation domain of NFκB; an Epstein-Barr virus R transactivator (Rta) activation domain; a tripartite activator comprising the VP64, the p65, and the Rta activation domains, the tripartite activator is known as a VPR activation domain; a histone acetyltransferase (HAT) core domain of the human E1A-associated protein p300, known as a p300 HAT core activation domain; a Krüppel associated box (KRAB) repression domain; a Repressor Element Silencing Transcription Factor (REST) repression domain; a WRPW motif (SEQ ID NO: 346) of the hairy-related basic helix-loop-helix repressor proteins, the motif is known as a WRPW repression domain (SEQ ID NO: 346); a DNA (cytosine-5)-methyltransferase 3B (DNMT3B) repression domain; and an HP1 alpha chromoshadow repression domain.

In some embodiments, the ACP is a small molecule (e.g., drug) inducible polypeptide. For example, in some embodiments, the ACP may be induced by tetracycline (or derivative thereof), and comprises a TetR domain and a VP16 effector domain. In some embodiments, the ACP includes an estrogen receptor variant, such as ERT2, and may be regulated by tamoxifen, or a metabolite thereof (such as 4-hydroxy-tamoxifen [4-OHT], N-desmethyltamoxifen, tamoxifen-N-oxide, or endoxifen), through tamoxifen-controlled nuclear localization. In some embodiments, the ACP comprises a nuclear-localization signal (NLS). In certain embodiments, the NLS comprises the amino acid sequence of MPKKKRKV (SEQ ID NO: 296). An exemplary nucleic acid sequence encoding SEQ ID NO: 296 is ATGCCCAAGAAGAAGCGGAAGGTT (SEQ ID NO: 297) or ATGCCCAAGAAAAAGCGGAAGGTG (SEQ ID NO: 340). In some embodiments, a nucleic acid sequence encoding SEQ ID NO: 296 may comprise SEQ ID NO: 297 or SEQ ID NO: 340, or comprises a sequence that is at least 90%, at least 91%, at least 92%, at least 93%, at least 94%, at least 95%, at least 96%, at least 97%, at least 98%, or at least 99% identical to SEQ ID NO: 297 or SEQ ID NO: 340.

In some embodiments, the ACP is a small molecule (e.g., drug) inducible polypeptide that includes a repressible protease and one or more cognate cleavage sites of the repressible protease. In some embodiments, a repressible protease is active (cleaves a cognate cleavage site) in the absence of the specific agent and is inactive (does not cleave a cognate cleavage site) in the presence of the specific agent. In some embodiments, the specific agent is a protease inhibitor. In some embodiments, the protease inhibitor specifically inhibits a given repressible protease of the present disclosure. The repressible protease can be any of the proteases described herein that is capable of inactivation by the presence or absence of a specific agent (see “Protease Cleavage Site” above for exemplary repressible proteases, cognate cleavage sites, and protease inhibitors).

In some embodiments, the ACP has a degron domain (see “Degron Systems and Domains” above for exemplary degron sequences). The degron domain can be in any order or position relative to the individual domains of the ACP. For example, the degron domain can be N-terminal of the repressible protease, C-terminal of the repressible protease, N-terminal of the ZF protein domain, C-terminal of the ZF protein domain, N-terminal of the effector domain, or C-terminal of the effector domain.

Exemplary sequences of components of ACPs and exemplary ACPs of the present disclosure are provided in Table 5D. In some embodiments, nucleic acids may comprise a sequence in Table 5D, or a nucleic acid sequence that is at least 90%, at least 910%, at least 92%, at least 93%, at least 94%, at least 95%, at least 96%, at least 97%, at least 98%, or at least 99% identical to a sequence in Table 5D.

TABLE 5D

| Design | Amino Acid Sequence | Nucleic Acid Sequence |
|---|---|---|
| NLS + miniVPR | MPKKKRKVDALDDFDLDMLGSDALD | ATGCCCAAGAAAAAGCGGAAGGTGG |
| activation domain | DFDLDMLGSDALDDFDLDMLGSDAL | ACGCCCTGGACGACTTCGATCTGGA |
| + NS3 protease | DDFDLDMLINSRSSGSPKKKRKVGS | TATGCTGGGCAGCGACGCTCTGGAT |
| ZFBD DNA | GGGSGGSGSVLPQAPAPAPAPAMVS | GATTTTGACCTGGACATGCTCGGCT |
| binding domain | ALAQAPAPVPVLAPGPPQAVAPPAP | CTGATGCACTCGACGATTTCGACCT |
| | KPTQAGEGTLSEALLQLQFDDEDLG | CGATATGTTGGGATCTGATGCCCTT |
| | ALLGNSTDPAVFTDLASVDNSEFQQ | GATGACTTTGATCTCGACATGTTGA |
| | LLNQGIPVAPHTTEPMLMEYPEAIT | TCAATAGCCGGTCCAGCGGCAGCCC |
| | RLVTGAQRPPDPAPAPLGAPGLPNG | CAAGAAGAAGAGAAAAGTCGGCTCT |
| | LLSGDEDFSSIADMDFSALLSGGGS | GGCGGCGGATCTGGCGGTTCTGGAT |
| | GGSGSDLSHPPPRGHLDELTTTLES | CTGTTTTGCCCCAAGCTCCTGCTCC |
| | MTEDLNLDSPLTPELNEILDTFLND | TGCACCAGCTCCAGCTATGGTTTCT |
| | ECLLHAMHISTGLSIFDTSLFEDVV | GCTCTGGCTCAGGCTCCAGCTCCTG |
| | CCHSIYGKKKGDIDTYRYIGSSGTG | TGCCTGTTCTTGCTCCTGGACCTCC |
| | CVVIVGRIVLSGSGTSAPITAYAQQ | TCAGGCTGTTGCTCCACCAGCACCT |
| | TRGLLGCIITSLTGRDKNQVEGEVQ | AAACCTACACAGGCCGGCGAGGGAA |
| | IVSTATQTFLATCINGVCWAVYHGA | CACTGTCTGAAGCTCTGCTGCAGCT |
| | GTRTIASPKGPVIQMYTNVDQDLVG | CCAGTTCGACGACGAAGATCTGGGA |
| | WPAPQGSRSLTPCTCGSSDLYLVTR | GCCCTGCTGGGCAATAGCACAGATC |
| | HADVIPVRRRGDSRGSLLSPRPISY | CTGCCGTGTTCACCGATCTGGCCAG |
| | LKGSSGGPLLCPAGHAVGLFRAAVC | CGTGGACAATAGCGAGTTCCAGCAG |
| | TRGVAKAVDFIPVENLETTMRSPVF | CTCCTGAACCAGGGCATTCCTGTGG |
| | TDNSSPPAVTLTHPITKIDREVLYQ | CTCCTCACACCACCGAGCCTATGCT |
| | EFDEMEECSQHMSRPGERPFQCRIC | GATGGAATACCCCGAGGCCATCACC |
| | MRNFSNMSNLTRHTRTHTGEKPFQC | AGACTGGTCACCGGTGCTCAAAGAC |
| | RICMRNFSDRSVLRRHLRTHTGSQK | CACCTGATCCGGCTCCAGCACCTCT |
| | PFQCRICMRNFSDPSNLARHTRTHT | TGGAGCACCTGGACTGCCTAATGGA |
| | GEKPFQCRICMRNFSDRSSLRRHLR | CTGCTGTCTGGCGACGAGGACTTCA |
| | THTGSQKPFQCRICMRNFSQSGTLH | GCTCTATCGCCGACATGGATTTCAG |
| | RHTRTHTGEKPFQCRICMRNFSQRP | CGCCCTGCTCAGTGGCGGTGGAAGC |
| | NLTRHLRTHLRGS | GGAGGAAGTGGCAGCGATCTTTCTC |
| | (SEQ ID NO: 301) | ACCCTCCACCTAGAGGCCACCTGGA |
| | | CGAGCTGACAACCACACTGGAATCC |
| | | ATGACCGAGGACCTGAACCTGGACA |
| | | GCCCTCTGACACCCGAGCTGAACGA |
| | | GATCCTGGACACCTTCCTGAACGAC |
| | | GAGTGTCTGCTGCACGCCATGCACA |
| | | TCTCTACCGGCCTGAGCATCTTCGA |
| | | CACCAGCCTGTTTGAGGATGTCGTG |
| | | TGCTGCCACAGCATCTACGGCAAGA |
| | | AGAAGGGCGACATCGACACCTACCG |
| | | GTACATCGGCAGCTCTGGCACAGGC |
| | | TGTGTGGTCATCGTGGGCAGAATCG |
| | | TGCTGTCTGGCAGCGGAACAAGCGC |
| | | CCCTATCACAGCCTATGCTCAGCAG |
| | | ACAAGAGGCCTGCTGGGCTGCATCA |
| | | TCACAAGCCTGACCGGCAGAGACAA |
| | | GAACCAGGTGGAAGGCGAGGTGCAG |
| | | ATCGTGTCTACAGCTACCCAGACCT |
| | | TCCTGGCCACCTGTATCAATGGCGT |
| | | GTGCTGGGCCGTGTATCACGGCGCT |
| | | GGAACCAGAACAATCGCCTCTCCTA |
| | | AGGGCCCCGTGATCCAGATGTACAC |
| | | CAACGTGGACCAGGACCTCGTTGGC |
| | | TGGCCTGCTCCTCAAGGCAGCAGAA |
| | | GCCTGACACCTTGCACCTGTGGCTC |
| | | CAGCGATCTGTACCTGGTCACCAGA |
| | | CACGCCGACGTGATCCCTGTCAGAA |
| | | GAAGAGGGGATTCCAGAGGCAGCCT |
| | | GCTGAGCCCTAGACCTATCAGCTAC |
| | | CTGAAGGGCTCTAGCGGCGGACCTC |
| | | TGCTTTGTCCTGCTGGACATGCCGT |
| | | GGGCCTGTTTAGAGCCGCCGTGTGT |
| | | ACAAGAGGCGTGGCCAAAGCCGTGG |
| | | ACTTCATCCCCGTGGAAAACCTGGA |
| | | AACCACCATGCGGAGCCCCGTGTTC |
| | | ACCGACAATTCTAGCCCTCCAGCCG |
| | | TGACACTGACACACCCCATCACCAA |
| | | GATCGACAGAGAGGTGCTGTACCAA |
| | | GAGTTCGACGAGATGGAAGAGTGCA |
| | | GCCAGCACATGTCTAGACCTGGCGA |
| | | GAGGCCCTTCCAGTGCCGGATCTGC |
| | | ATGCGGAACTTCAGCAACATGAGCA |
| | | ACCTGACCAGACACACCCGGACACA |
| | | CACAGGCGAGAAGCCTTTTCAGTGC |
| | | AGAATCTGTATGCGCAATTTCTCCG |
| | | ACAGAAGCGTGCTGCGGAGACACCT |
| | | GAGAACCCACACCGGCAGCCAGAAA |
| | | CCATTCCAGTGTCGCATCTGTATGA |
| | | GAAACTTTAGCGACCCCTCCAATCT |
| | | GGCCCGGCACACCAGAACACATACC |
| | | GGGGAAAAACCCTTTCAGTGTAGGA |
| | | TATGCATGAGGAATTTTTCCGACCG |
| | | GTCCAGCCTGAGGCGGCACCTGAGG |
| | | ACACATACTGGCTCCCAAAAGCCGT |
| | | TCCAATGTCGGATATGTATGCGCAA |
| | | CTTTAGCCAGAGCGGCACCCTGCAC |
| | | AGACACACAAGAACCCATACTGGCG |
| | | AGAAACCTTTCCAATGTAGAATCTG |
| | | CATGCGAAATTTTTCCCAGCGGCCT |
| | | AATCTGACCAGGCATCTGAGGACCC |
| | | ACCTGAGAGGATCT |
| | | (SEQ ID NO: 306) |
| NLS + ZFBD | MPKKKRKVMSRPGERPFQCRICMRN | ATGCCCAAGAAAAAGCGGAAGGTGA |
| DNA binding | FSNMSNLTRHTRTHTGEKPFQCRIC | TGTCTAGACCTGGCGAGAGGCCCTT |
| domain + NS3 | MRNFSDRSVLRRHLRTHTGSQKPFQ | CCAGTGCCGGATCTGCATGCGGAAC |
| protease | CRICMRNFSDPSNLARHTRTHTGEK | TTCAGCAACATGAGCAACCTGACCA |
| mini VPR | PFQCRICMRNFSDRSSLRRHLRTHT | GACACACCCGGACACACACAGGCGA |
| activation domain | GSQKPFQCRICMRNFSQSGTLHRHT | GAAGCCTTTTCAGTGCAGAATCTGT |
| | RTHTGEKPFQCRICMRNFSQRPNLT | ATGCGCAATTTCTCCGACAGAAGCG |
| | RHLRTHLRGSEDVVCCHSIYGKKKG | TGCTGCGGAGACACCTGAGAACCCA |
| | DIDTYRYIGSSGTGCVVIVGRIVLS | CACCGGCAGCCAGAAACCATTCCAG |
| | GSGTSAPITAYAQQTRGLLGCIITS | TGTCGCATCTGTATGAGAAACTTTA |
| | LTGRDKNQVEGEVQIVSTATQTFLA | GCGACCCCTCCAATCTGGCCCGGCA |
| | TCINGVCWAVYHGAGTRTIASPKGP | CACCAGAACACATACCGGGGAAAAA |
| | VIQMYTNVDQDLVGWPAPQGSRSLT | CCCTTTCAGTGTAGGATATGCATGA |
| | PCTCGSSDLYLVTRHADVIPVRRRG | GGAATTTTTCCGACCGGTCCAGCCT |
| | DSRGSLLSPRPISYLKGSSGGPLLC | GAGGCGGCACCTGAGGACACATACT |
| | PAGHAVGLFRAAVCTRGVAKAVDFI | GGCTCCCAAAAGCCGTTCCAATGTC |
| | PVENLETTMRSPVFTDNSSPPAVTL | GGATATGTATGCGCAACTTTAGCCA |
| | THPITKIDREVLYQEFDEMEECSQH | GAGCGGCACCCTGCACAGACACACA |
| | DALDDFDLDMLGSDALDDFDLDMLG | AGAACCCATACTGGCGAGAAACCTT |
| | SDALDDFDLDMLGSDALDDFDLDML | TCCAATGTAGAATCTGCATGCGAAA |
| | INSRSSGSPKKKRKVGSGGGSGGSG | TTTTTCCCAGCGGCCTAATCTGACC |
| | SVLPQAPAPAPAPAMVSALAQAPAP | AGGCATCTGAGGACCCACCTGAGAG |
| | VPVLAPGPPQAVAPPAPKPTQAGEG | GATCTGAGGATGTCGTGTGCTGCCA |
| | TLSEALLQLQFDDEDLGALLGNSTD | CAGCATCTACGGCAAGAAGAAGGGC |
| | PAVFTDLASVDNSEFQQLLNQGIPV | GACATCGACACCTACCGGTACATCG |
| | APHTTEPMLMEYPEAITRLVTGAQR | GCAGCTCTGGCACAGGCTGTGTGGT |
| | PPDPAPAPLGAPGLPNGLLSGDEDF | CATCGTGGGCAGAATCGTGCTGTCT |
| | SSIADMDFSALLSGGGSGGSGSDLS | GGCAGCGGAACAAGCGCCCCTATCA |
| | HPPPRGHLDELTTTLESMTEDLNLD | CAGCCTATGCTCAGCAGACAAGAGG |
| | SPLTPELNEILDTFLNDECLLHAMH | CCTGCTGGGCTGCATCATCACAAGC |
| | ISTGLSIFDTSLF | CTGACCGGCAGAGACAAGAACCAGG |
| | (SEQ ID NO: 302) | TGGAAGGCGAGGTGCAGATCGTGTC |
| | | TACAGCTACCCAGACCTTCCTGGCC |
| | | ACCTGTATCAATGGCGTGTGCTGGG |
| | | CCGTGTATCACGGCGCTGGAACCAG |
| | | AACAATCGCCTCTCCTAAGGGCCCC |
| | | GTGATCCAGATGTACACCAACGTGG |
| | | ACCAGGACCTCGTTGGCTGGCCTGC |
| | | TCCTCAAGGCAGCAGAAGCCTGACA |
| | | CCTTGCACCTGTGGCTCCAGCGATC |
| | | TGTACCTGGTCACCAGACACGCCGA |
| | | CGTGATCCCTGTCAGAAGAAGAGGG |
| | | GATTCCAGAGGCAGCCTGCTGAGCC |
| | | CTAGACCTATCAGCTACCTGAAGGG |
| | | CTCTAGCGGCGGACCTCTGCTTTGT |
| | | CCTGCTGGACATGCCGTGGGCCTGT |
| | | TTAGAGCCGCCGTGTGTACAAGAGG |
| | | CGTGGCCAAAGCCGTGGACTTCATC |
| | | CCCGTGGAAAACCTGGAAACCACCA |
| | | TGCGGAGCCCCGTGTTCACCGACAA |
| | | TTCTAGCCCTCCAGCCGTGACACTG |
| | | ACACACCCCATCACCAAGATCGACA |
| | | GAGAGGTGCTGTACCAAGAGTTCGA |
| | | CGAGATGGAAGAGTGCAGCCAGCAC |
| | | GACGCCCTGGACGACTTCGATCTGG |
| | | ATATGCTGGGCAGCGACGCTCTGGA |
| | | TGATTTTGACCTGGACATGCTCGGC |
| | | TCTGATGCACTCGACGATTTCGACC |
| | | TCGATATGTTGGGATCTGATGCCCT |
| | | TGATGACTTTGATCTCGACATGTTG |
| | | ATCAATAGCCGGTCCAGCGGCAGCC |
| | | CCAAGAAGAAGAGAAAAGTCGGCTC |
| | | TGGCGGCGGATCTGGCGGTTCTGGA |
| | | TCTGTTTTGCCCCAAGCTCCTGCTC |
| | | CTGCACCAGCTCCAGCTATGGTTTC |
| | | TGCTCTGGCTCAGGCTCCAGCTCCT |
| | | GTGCCTGTTCTTGCTCCTGGACCTC |
| | | CTCAGGCTGTTGCTCCACCAGCACC |
| | | TAAACCTACACAGGCCGGCGAGGGA |
| | | ACACTGTCTGAAGCTCTGCTGCAGC |
| | | TCCAGTTCGACGACGAAGATCTGGG |
| | | AGCCCTGCTGGGCAATAGCACAGAT |
| | | CCTGCCGTGTTCACCGATCTGGCCA |
| | | GCGTGGACAATAGCGAGTTCCAGCA |
| | | GCTCCTGAACCAGGGCATTCCTGTG |
| | | GCTCCTCACACCACCGAGCCTATGC |
| | | TGATGGAATACCCCGAGGCCATCAC |
| | | CAGACTGGTCACCGGTGCTCAAAGA |
| | | CCACCTGATCCGGCTCCAGCACCTC |
| | | TTGGAGCACCTGGACTGCCTAATGG |
| | | ACTGCTGTCTGGCGACGAGGACTTC |
| | | AGCTCTATCGCCGACATGGATTTCA |
| | | GCGCCCTGCTCAGTGGCGGTGGAAG |
| | | CGGAGGAAGTGGCAGCGATCTTTCT |
| | | CACCCTCCACCTAGAGGCCACCTGG |
| | | ACGAGCTGACAACCACACTGGAATC |
| | | CATGACCGAGGACCTGAACCTGGAC |
| | | AGCCCTCTGACACCCGAGCTGAACG |
| | | AGATCCTGGACACCTTCCTGAACGA |
| | | CGAGTGTCTGCTGCACGCCATGCAC |
| | | ATCTCTACCGGCCTGAGCATCTTCG |
| | | ACACCAGCCTGTTT |
| | | (SEQ ID NO: 305) |
| NLS + ZFBD | MPKKKRKVSRPGERPFQCRICMRNF | ATGCCCAAGAAGAAGCGGAAGGTTT |
| DNA binding | SRRHGLDRHTRTHTGEKPFQCRICM | CCCGGCCTGGCGAGAGGCCTTTCCA |
| domain + NS3 | RNFSDHSSLKRHLRTHTGSQKPFQC | GTGCAGAATCTGCATGCGGAACTTC |
| protease | RICMRNFSVRHNLTRHLRTHTGEKP | AGCAGACGGCACGGCCTGGACAGAC |
| mini VPR | FQCRICMRNFSDHSNLSRHLKTHTG | ACACCAGAACACACACAGGCGAGAA |
| activation domain | SQKPFQCRICMRNFSQRSSLVRHLR | ACCCTTCCAGTGCCGGATCTGTATG |
| | THTGEKPFQCRICMRNFSESGHLKR | AGAAATTTCAGCGACCACAGCAGCC |
| | HLRTHLRGSEDVVCCHSIYGKKKGD | TGAAGCGGCACCTGAGAACCCATAC |
| | IDTYRYIGSSGTGCVVIVGRIVLSG | CGGCAGCCAGAAACCATTTCAGTGT |
| | SGTSAPITAYAQQTRGLLGCIITSL | AGGATATGCATGCGCAATTTCTCCG |
| | TGRDKNQVEGEVQIVSTATQTFLAT | TGCGGCACAACCTGACCAGACACCT |
| | CINGVCWAVYHGAGTRTIASPKGPV | GAGGACACACACCGGGGAGAAGCCT |
| | IQMYTNVDQDLVGWPAPQGSRSLTP | TTTCAATGTCGCATATGCATGAGAA |
| | CTCGSSDLYLVTRHADVIPVRRRGD | ACTTCTCTGACCACTCCAACCTGAG |
| | SRGSLLSPRPISYLKGSSGGPLLCP | CCGCCACCTCAAAACCCACACCGGC |
| | AGHAVGLFRAAVCTRGVAKAVDFIP | TCTCAAAAGCCCTTCCAATGTAGAA |
| | VENLETTMRSPVFTDNSSPPAVTLT | TATGTATGAGGAACTTTAGCCAGCG |
| | HPITKIDREVLYQEFDEMEECSQHD | GAGCAGCCTCGTGCGCCATCTGAGA |
| | ALDDFDLDMLGSDALDDFDLDMLGS | ACTCACACTGGCGAAAAGCCGTTTC |
| | DALDDFDLDMLGSDALDDFDLDMLI | AATGCCGTATCTGTATGCGCAACTT |
| | NSRSSGSPKKKRKVGSGGGSGGSGS | TAGCGAGAGCGGCCACCTGAAGAGA |
| | VLPQAPAPAPAPAMVSALAQAPAPV | CATCTGCGCACACACCTGAGAGGCA |
| | PVLAPGPPQAVAPPAPKPTQAGEGT | GCGAGGATGTCGTGTGCTGCCACAG |
| | LSEALLQLQFDDEDLGALLGNSTDP | CATCTACGGAAAGAAGAAGGGCGAC |
| | AVFTDLASVDNSEFQQLLNQGIPVA | ATCGACACCTATCGGTACATCGGCA |
| | PHTTEPMLMEYPEAITRLVTGAQRP | GCAGCGGCACAGGCTGTGTTGTGAT |
| | PDPAPAPLGAPGLPNGLLSGDEDFS | CGTGGGCAGAATCGTGCTGAGCGGC |
| | SIADMDFSALLSGGGSGGSGSDLSH | TCTGGAACAAGCGCCCCTATCACAG |
| | PPPRGHLDELTTTLESMTEDLNLDS | CCTACGCTCAGCAGACAAGAGGCCT |
| | PLTPELNEILDTFLNDECLLHAMHI | GCTGGGCTGCATCATCACAAGCCTG |
| | STGLSIFDTSLF | ACCGGCAGAGACAAGAACCAGGTGG |
| | (SEQ ID NO: 303) | AAGGCGAGGTGCAGATCGTGTCTAC |
| | | AGCTACCCAGACCTTCCTGGCCACC |
| | | TGTATCAATGGCGTGTGCTGGGCCG |
| | | TGTATCACGGCGCTGGCACAAGAAC |
| | | AATCGCCTCTCCAAAGGGCCCCGTG |
| | | ATCCAGATGTACACCAACGTGGACC |
| | | AGGACCTCGTTGGCTGGCCTGCTCC |
| | | TCAAGGCAGCAGAAGCCTGACACCT |
| | | TGCACCTGTGGCTCCAGCGATCTGT |
| | | ACCTGGTCACCAGACACGCCGACGT |
| | | GATCCCTGTCAGAAGAAGAGGGGAT |
| | | TCCAGAGGCAGCCTGCTGAGCCCTA |
| | | GACCTATCAGCTACCTGAAGGGCAG |
| | | CTCTGGCGGACCTCTGCTTTGTCCT |
| | | GCTGGACATGCCGTGGGCCTGTTTA |
| | | GAGCCGCCGTGTGTACAAGAGGCGT |
| | | GGCCAAAGCCGTGGACTTCATCCCC |
| | | GTGGAAAACCTGGAAACCACCATGC |
| | | GGAGCCCCGTGTTCACCGACAATTC |
| | | TAGCCCTCCAGCCGTGACACTGACA |
| | | CACCCCATCACCAAGATCGACAGAG |
| | | AGGTGCTGTACCAAGAGTTCGACGA |
| | | GATGGAAGAGTGCAGCCAGCACGAC |
| | | GCTCTTGATGACTTTGACCTGGATA |
| | | TGCTCGGATCAGATGCCCTGGACGA |
| | | TTTCGATCTGGACATGTTGGGGTCT |
| | | GATGCTCTCGACGACTTCGATCTGG |
| | | ATATGCTTGGAAGTGACGCGCTGGA |
| | | TGATTTCGACCTTGACATGCTCATC |
| | | AATTCTCGATCCAGTGGAAGCCCGA |
| | | AAAAGAAACGCAAGGTGGGAAGTGG |
| | | GGGCGGCTCCGGTGGGAGCGGTAGT |
| | | GTATTGCCTCAAGCTCCCGCGCCCG |
| | | CTCCTGCTCCGGCAATGGTTTCAGC |
| | | TCTGGCACAAGCTCCAGCTCCAGTG |
| | | CCTGTGCTCGCCCCTGGCCCTCCGC |
| | | AGGCCGTAGCACCTCCCGCCCCCAA |
| | | ACCGACGCAAGCCGGTGAGGGGACT |
| | | CTCTCTGAAGCCTTGCTGCAGCTTC |
| | | AGTTCGATGATGAAGATCTGGGCGC |
| | | GCTCTTGGGGAACAGCACGGATCCG |
| | | GCAGTATTTACGGACCTCGCATCAG |
| | | TTGACAATAGTGAATTTCAACAACT |
| | | TCTTAACCAGGGAATACCGGTTGCG |
| | | CCCCATACGACGGAACCTATGCTGA |
| | | TGGAGTACCCTGAAGCTATAACCAG |
| | | ACTCGTAACTGGCGCCCAACGCCCG |
| | | CCCGACCCGGCTCCTGCGCCGCTGG |
| | | GTGCGCCGGGTCTTCCGAATGGTCT |
| | | TCTCTCAGGGGACGAAGATTTCAGT |
| | | TCCATTGCGGATATGGACTTTTCCG |
| | | CGCTCCTGAGTGGGGGTGGCTCTGG |
| | | AGGCTCTGGTTCCGACCTCAGCCAT |
| | | CCTCCACCGAGAGGACACCTCGACG |
| | | AGCTGACAACCACCCTCGAAAGTAT |
| | | GACGGAAGATCTGAACTTGGATTCC |
| | | CCCCTTACCCCAGAACTGAATGAAA |
| | | TCCTCGATACGTTCTTGAACGATGA |
| | | GTGCCTTTTGCACGCCATGCATATA |
| | | TCAACAGGTTTGTCTATCTTCGACA |
| | | CGTCCCTCTTTTGA |
| | | (SEQ ID NO: 304) |
| mini VPR | DALDDFDLDMLGSDALDDFDLDMLG | GACGCCCTGGACGACTTCGATCTGG |
| activator domain | SDALDDFDLDMLGSDALDDFDLDML | ATATGCTGGGCAGCGACGCTCTGGA |
| | INSRSSGSPKKKRKVGSGGGSGGSG | TGATTTTGACCTGGACATGCTCGGC |
| | SVLPQAPAPAPAPAMVSALAQAPAP | TCTGATGCACTCGACGATTTCGACC |
| | VPVLAPGPPQAVAPPAPKPTQAGEG | TCGATATGTTGGGATCTGATGCCCT |
| | TLSEALLQLQFDDEDLGALLGNSTD | TGATGACTTTGATCTCGACATGTTG |
| | PAVFTDLASVDNSEFQQLLNQGIPV | ATCAATAGCCGGTCCAGCGGCAGCC |
| | APHTTEPMLMEYPEAITRLVTGAQR | CCAAGAAGAAGAGAAAAGTCGGCTC |
| | PPDPAPAPLGAPGLPNGLLSGDEDF | TGGCGGCGGATCTGGCGGTTCTGGA |
| | SSIADMDFSALLSGGGSGGSGSDLS | TCTGTTTTGCCCCAAGCTCCTGCTC |
| | HPPPRGHLDELTTTLESMTEDLNLD | CTGCACCAGCTCCAGCTATGGTTTC |
| | SPLTPELNEILDTFLNDECLLHAMH | TGCTCTGGCTCAGGCTCCAGCTCCT |
| | ISTGLSIFDTSLF | GTGCCTGTTCTTGCTCCTGGACCTC |
| | (SEQ ID NO: 325) | CTCAGGCTGTTGCTCCACCAGCACC |
| | | TAAACCTACACAGGCCGGCGAGGGA |
| | | ACACTGTCTGAAGCTCTGCTGCAGC |
| | | TCCAGTTCGACGACGAAGATCTGGG |
| | | AGCCCTGCTGGGCAATAGCACAGAT |
| | | CCTGCCGTGTTCACCGATCTGGCCA |
| | | GCGTGGACAATAGCGAGTTCCAGCA |
| | | GCTCCTGAACCAGGGCATTCCTGTG |
| | | GCTCCTCACACCACCGAGCCTATGC |
| | | TGATGGAATACCCCGAGGCCATCAC |
| | | CAGACTGGTCACCGGTGCTCAAAGA |
| | | CCACCTGATCCGGCTCCAGCACCTC |
| | | TTGGAGCACCTGGACTGCCTAATGG |
| | | ACTGCTGTCTGGCGACGAGGACTTC |
| | | AGCTCTATCGCCGACATGGATTTCA |
| | | GCGCCCTGCTCAGTGGCGGTGGAAG |
| | | CGGAGGAAGTGGCAGCGATCTTTCT |
| | | CACCCTCCACCTAGAGGCCACCTGG |
| | | ACGAGCTGACAACCACACTGGAATC |
| | | CATGACCGAGGACCTGAACCTGGAC |
| | | AGCCCTCTGACACCCGAGCTGAACG |
| | | AGATCCTGGACACCTTCCTGAACGA |
| | | CGAGTGTCTGCTGCACGCCATGCAC |
| | | ATCTCTACCGGCCTGAGCATCTTCG |
| | | ACACCAGCCTGTTT |
| | | (SEQ ID NO: 322) |
| | | OR |
| | | GACGCTCTTGATGACTTTGACCTGG |
| | | ATATGCTCGGATCAGATGCCCTGGA |
| | | CGATTTCGATCTGGACATGTTGGGG |
| | | TCTGATGCTCTCGACGACTTCGATC |
| | | TGGATATGCTTGGAAGTGACGCGCT |
| | | GGATGATTTCGACCTTGACATGCTC |
| | | ATCAATTCTCGATCCAGTGGAAGCC |
| | | CGAAAAAGAAACGCAAGGTGGGAAG |
| | | TGGGGGCGGCTCCGGTGGGAGCGGT |
| | | AGTGTATTGCCTCAAGCTCCCGCGC |
| | | CCGCTCCTGCTCCGGCAATGGTTTC |
| | | AGCTCTGGCACAAGCTCCAGCTCCA |
| | | GTGCCTGTGCTCGCCCCTGGCCCTC |
| | | CGCAGGCCGTAGCACCTCCCGCCCC |
| | | CAAACCGACGCAAGCCGGTGAGGGG |
| | | ACTCTCTCTGAAGCCTTGCTGCAGC |
| | | TTCAGTTCGATGATGAAGATCTGGG |
| | | CGCGCTCTTGGGGAACAGCACGGAT |
| | | CCGGCAGTATTTACGGACCTCGCAT |
| | | CAGTTGACAATAGTGAATTTCAACA |
| | | ACTTCTTAACCAGGGAATACCGGTT |
| | | GCGCCCCATACGACGGAACCTATGC |
| | | TGATGGAGTACCCTGAAGCTATAAC |
| | | CAGACTCGTAACTGGCGCCCAACGC |
| | | CCGCCCGACCCGGCTCCTGCGCCGC |
| | | TGGGTGCGCCGGGTCTTCCGAATGG |
| | | TCTTCTCTCAGGGGACGAAGATTTC |
| | | AGTTCCATTGCGGATATGGACTTTT |
| | | CCGCGCTCCTGAGTGGGGGTGGCTC |
| | | TGGAGGCTCTGGTTCCGACCTCAGC |
| | | CATCCTCCACCGAGAGGACACCTCG |
| | | ACGAGCTGACAACCACCCTCGAAAG |
| | | TATGACGGAAGATCTGAACTTGGAT |
| | | TCCCCCCTTACCCCAGAACTGAATG |
| | | AAATCCTCGATACGTTCTTGAACGA |
| | | TGAGTGCCTTTTGCACGCCATGCAT |
| | | ATATCAACAGGTTTGTCTATCTTCG |
| | | ACACGTCCCTCTTTTGA |
| | | (SEQ ID NO: 343) |
| ZF5-7 Zinc finger | MSRPGERPFQCRICMRNFSNMSNLT | ATGTCTAGACCTGGCGAGAGGCCCT |
| domain | RHTRTHTGEKPFQCRICMRNFSDRS | TCCAGTGCCGGATCTGCATGCGGAA |
| | VLRRHLRTHTGSQKPFQCRICMRNF | CTTCAGCAACATGAGCAACCTGACC |
| | SDPSNLARHTRTHTGEKPFQCRICM | AGACACACCCGGACACACACAGGCG |
| | RNFSDRSSLRRHLRTHTGSQKPFQC | AGAAGCCTTTTCAGTGCAGAATCTG |
| | RICMRNFSQSGTLHRHTRTHTGEKP | TATGCGCAATTTCTCCGACAGAAGC |
| | FQCRICMRNFSQRPNLTRHLRTHLR | GTGCTGCGGAGACACCTGAGAACCC |
| | GS | ACACCGGCAGCCAGAAACCATTCCA |
| | (SEQ ID NO: 320) | GTGTCGCATCTGTATGAGAAACTTT |
| | | AGCGACCCCTCCAATCTGGCCCGGC |
| | | ACACCAGAACACATACCGGGGAAAA |
| | | ACCCTTTCAGTGTAGGATATGCATG |
| | | AGGAATTTTTCCGACCGGTCCAGCC |
| | | TGAGGCGGCACCTGAGGACACATAC |
| | | TGGCTCCCAAAAGCCGTTCCAATGT |
| | | CGGATATGTATGCGCAACTTTAGCC |
| | | AGAGCGGCACCCTGCACAGACACAC |
| | | AAGAACCCATACTGGCGAGAAACCT |
| | | TTCCAATGTAGAATCTGCATGCGAA |
| | | ATTTTTCCCAGCGGCCTAATCTGAC |
| | | CAGGCATCTGAGGACCCACCTGAGA |
| | | GGATCT |
| | | (SEQ ID NO: 323) |
| NS3 protease | EDVVCCHSIYGKKKGDIDTYRYIGS | GAGGATGTCGTGTGCTGCCACAGCA |
| | SGTGCVVIVGRIVLSGSGTSAPITA | TCTACGGCAAGAAGAAGGGCGACAT |
| | YAQQTRGLLGCIITSLTGRDKNQVE | CGACACCTACCGGTACATCGGCAGC |
| | GEVQIVSTATQTFLATCINGVCWAV | TCTGGCACAGGCTGTGTGGTCATCG |
| | YHGAGTRTIASPKGPVIQMYTNVDQ | TGGGCAGAATCGTGCTGTCTGGCAG |
| | DLVGWPAPQGSRSLTPCTCGSSDLY | CGGAACAAGCGCCCCTATCACAGCC |
| | LVTRHADVIPVRRRGDSRGSLLSPR | TATGCTCAGCAGACAAGAGGCCTGC |
| | PISYLKGSSGGPLLCPAGHAVGLFR | TGGGCTGCATCATCACAAGCCTGAC |
| | AAVCTRGVAKAVDFIPVENLETTMR | CGGCAGAGACAAGAACCAGGTGGAA |
| | SPVFTDNSSPPAVTLTHPITKIDRE | GGCGAGGTGCAGATCGTGTCTACAG |
| | VLYQEFDEMEECSQH | CTACCCAGACCTTCCTGGCCACCTG |
| | (SEQ ID NO: 321) | TATCAATGGCGTGTGCTGGGCCGTG |
| | | TATCACGGCGCTGGAACCAGAACAA |
| | | TCGCCTCTCCTAAGGGCCCCGTGAT |
| | | CCAGATGTACACCAACGTGGACCAG |
| | | GACCTCGTTGGCTGGCCTGCTCCTC |
| | | AAGGCAGCAGAAGCCTGACACCTTG |
| | | CACCTGTGGCTCCAGCGATCTGTAC |
| | | CTGGTCACCAGACACGCCGACGTGA |
| | | TCCCTGTCAGAAGAAGAGGGGATTC |
| | | CAGAGGCAGCCTGCTGAGCCCTAGA |
| | | CCTATCAGCTACCTGAAGGGCTCTA |
| | | GCGGCGGACCTCTGCTTTGTCCTGC |
| | | TGGACATGCCGTGGGCCTGTTTAGA |
| | | GCCGCCGTGTGTACAAGAGGCGTGG |
| | | CCAAAGCCGTGGACTTCATCCCCGT |
| | | GGAAAACCTGGAAACCACCATGCGG |
| | | AGCCCCGTGTTCACCGACAATTCTA |
| | | GCCCTCCAGCCGTGACACTGACACA |
| | | CCCCATCACCAAGATCGACAGAGAG |
| | | GTGCTGTACCAAGAGTTCGACGAGA |
| | | TGGAAGAGTGCAGCCAGCAC |
| | | (SEQ ID NO: 195) |
| | | OR |
| | | GAGGATGTCGTGTGCTGCCACAGCA |
| | | TCTACGGAAAGAAGAAGGGCGACAT |
| | | CGACACCTATCGGTACATCGGCAGC |
| | | AGCGGCACAGGCTGTGTTGTGATCG |
| | | TGGGCAGAATCGTGCTGAGCGGCTC |
| | | TGGAACAAGCGCCCCTATCACAGCC |
| | | TACGCTCAGCAGACAAGAGGCCTGC |
| | | TGGGCTGCATCATCACAAGCCTGAC |
| | | CGGCAGAGACAAGAACCAGGTGGAA |
| | | GGCGAGGTGCAGATCGTGTCTACAG |
| | | CTACCCAGACCTTCCTGGCCACCTG |
| | | TATCAATGGCGTGTGCTGGGCCGTG |
| | | TATCACGGCGCTGGCACAAGAACAA |
| | | TCGCCTCTCCAAAGGGCCCCGTGAT |
| | | CCAGATGTACACCAACGTGGACCAG |
| | | GACCTCGTTGGCTGGCCTGCTCCTC |
| | | AAGGCAGCAGAAGCCTGACACCTTG |
| | | CACCTGTGGCTCCAGCGATCTGTAC |
| | | CTGGTCACCAGACACGCCGACGTGA |
| | | TCCCTGTCAGAAGAAGAGGGGATTC |
| | | CAGAGGCAGCCTGCTGAGCCCTAGA |
| | | CCTATCAGCTACCTGAAGGGCAGCT |
| | | CTGGCGGACCTCTGCTTTGTCCTGC |
| | | TGGACATGCCGTGGGCCTGTTTAGA |
| | | GCCGCCGTGTGTACAAGAGGCGTGG |
| | | CCAAAGCCGTGGACTTCATCCCCGT |
| | | GGAAAACCTGGAAACCACCATGCGG |
| | | AGCCCCGTGTTCACCGACAATTCTA |
| | | GCCCTCCAGCCGTGACACTGACACA |
| | | CCCCATCACCAAGATCGACAGAGAG |
| | | GTGCTGTACCAAGAGTTCGACGAGA |
| | | TGGAAGAGTGCAGCCAGCAC |
| | | (SEQ ID NO: 342) |

### Multicistronic and Multiple Promoter Systems

In some embodiments, engineered nucleic acids (e.g., an engineered nucleic acid comprising an expression cassette) are configured to produce multiple proteins (e.g., a cytokine, CAR, ACP, membrane-cleavable chimeric protein, and/or combinations thereof). For example, nucleic acids may be configured to produce 2-20 different proteins. In some embodiments, nucleic acids are configured to produce 2-20, 2-19, 2-18, 2-17, 2-16, 2-15, 2-14, 2-13, 2-12, 2-11,2-10,2-9,2-8,2-7,2-6,2-5,2-4,2-3,3-20,3-19,3-18, 3-17, 3-16, 3-15, 3-14, 3-13, 3-12, 3-11,3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17,11-16,11-15, 11-14, 11-13,11-12,12-20, 12-19, 12-18,12-17,12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 proteins. In some embodiments, nucleic acids are configured to produce 1, 2, 3, 4, 5, 6, 7, 8, 9, 10, 11, 12, 13, 14, 15, 16, 17, 18, 19, or 20 proteins.

In some embodiments, engineered nucleic acids can be multicistronic, i.e., more than one separate polypeptide (e.g., multiple proteins, such as a cytokine, CAR, ACP, and/or membrane-cleavable chimeric protein described herein) can be produced from a single mRNA transcript. In some embodiments, a multicistronic engineered nucleic acid of disclosure can be configured to encode a cytokine, CAR, and membrane-cleavable chimeric protein described herein. For example, a multicistronic engineered nucleic acid of disclosure can be configured to encode a cytokine, an aCAR, and membrane-cleavable chimeric protein described herein. For example, a multicistronic engineered nucleic acid of disclosure can be configured to encode a cytokine, an aCAR, an iCAR, and membrane-cleavable chimeric protein described herein.

Engineered nucleic acids can be multicistronic through the use of various linkers, e.g., a polynucleotide sequence encoding a first protein can be linked to a nucleotide sequence encoding a second protein, such as in a first gene:linker:second gene 5′ to 3′ orientation. A linker can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage. In some embodiments, an engineered nucleic acid disclosed herein comprises an E2A/T2A ribosome skipping element. In certain embodiments, the E2A/T2A ribosome skipping element comprises the amino acid sequence of GSGQCTNYALLKLAGDVESNPGPGSGEGRGSLLTCGDVEENPGP (SEQ ID NO: 281). An exemplary nucleic acid encoding SEQ ID NO: 281 is GGTAGCGGCCAGTGTACCAACTACGCCCTGCTGAAACTGGCCGGCGACGTGGAATC TAATCCTGGACCTGGATCTGGCGAGGGACGCGGGAGTCTACTGACGTGTGGAGACG TGGAGGAAAACCCTGGACCT (SEQ ID NO: 282). In certain embodiments, a nucleic acid encoding SEQ ID NO: 281 comprises a sequence that is at least 90%, at least 91%, at least 92%, at least 93%, at least 94%, at least 95%, at least 96%, at least 97%, at least 98%, or at least 99% identical to SEQ ID NO: 282. In some embodiments, an engineered nucleic acid disclosed herein comprises an E2A/T2A ribosome skipping element. In certain embodiments, the E2A/T2A ribosome skipping element comprises the amino acid sequence of QCTNYALLKLAGDVESNPGPGSGEGRGSLLTCGDVEENPGP (SEQ ID NO: 283). An exemplary nucleic acid encoding SEQ ID NO: 283 is CAGTGTACCAACTACGCCCTGCTGAAACTGGCCGGCGACGTGGAATCTAATCCTGG ACCTGGATCTGGCGAGGGACGCGGGAGTCTACTGACGTGTGGAGACGTGGAGGAA AACCCTGGACCT (SEQ ID NO: 284). In certain embodiments, a nucleic acid encoding SEQ ID NO: 283 comprises a sequence that is at least 90%, at least 91%, at least 92%, at least 93%, at least 94%, at least 95%, at least 96%, at least 97%, at least 98%, or at least 99% identical to SEQ ID NO: 284.

Other suitable linkers comprising 2A ribosome skipping elements are shown in the Table below.

2A containing linkers

| | Nt | AA |
|---|---|---|
| 2A_C | GGTTCTGGCCAGTGCACAAA | GSGQCTNYALLKLAGDVESN |
| | TTATGCACTGCTGAAGCTCG | PGPGSGEGRGSLLTCGDVEE |
| | CCGGGGATGTCGAGAGTAAC | NPGP |
| | CCAGGACCTGGAAGCGGAGA | (SEQ ID NO: 401) |
| | AGGTCGTGGTAGTCTACTAA | |
| | CGTGTGGTGATGTAGAAGAA | |
| | AATCCTGGACCT | |
| | (SEQ ID NO: 398) | |
| 2A_B | GGTAGCGGCCAGTGTACCAA | GSGQCTNYALLKLAGDVESN |
| | CTACGCCCTGCTGAAACTGG | PGPGSGEGRGSLLTCGDVEE |
| | CCGGCGACGTGGAATCTAAT | NPGP |
| | CCTGGACCTGGATCTGGCGA | (SEQ ID NO: 402) |
| | GGGACGCGGGAGTCTACTGA | |
| | CGTGTGGAGACGTGGAGGAA | |
| | AACCCTGGACCT | |
| | (SEQ ID NO: 399) | |
| 2A_A | GGCTCTGGCCAGTGCACCAA | GSGQCTNYALLKLAGDVESN |
| | CTACGCCCTGCTTAAACTGG | PGPGSGEGRGSLLTCGDVEE |
| | CCGGCGACGTGGAATCCAAT | NPGP |
| | CCTGGACCTGGATCTGGCGA | (SEQ ID NO: 403) |
| | GGGACGCGGGAGTCTACTGA | |
| | CGTGTGGAGACGTGGAGGAA | |
| | AACCCTGGACCT | |
| | (SEQ ID NO: 400) | |

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker of the present disclosure is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third protein, each separated by linkers such that separate polypeptides encoded by the first, second, and third proteins are produced).

Engineered nucleic acids can use multiple promoters to express genes from multiple ORFs, i.e., more than one separate mRNA transcript can be produced from a single engineered nucleic acid. For example, a first promoter can be operably linked to a polynucleotide sequence encoding a first protein, and a second promoter can be operably linked to a polynucleotide sequence encoding a second protein. In general, any number of promoters can be used to express any number of proteins. In some embodiments, at least one of the ORFs expressed from the multiple promoters can be multicistronic.

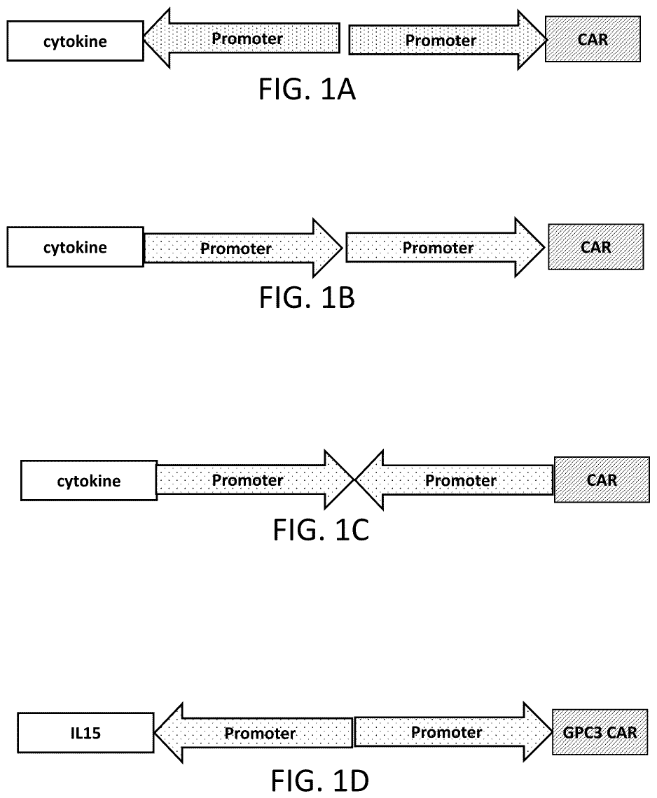

Expression cassettes encoded on the same engineered nucleic acid can be oriented in any manner suitable for expression of the encoded exogenous polynucleotide sequences. Expression cassettes encoded on the same engineered nucleic acid can be oriented in the same direction, i.e., transcription of separate cassettes proceeds in the same direction. Constructs oriented in the same direction can be organized in a head-to-tail format referring to the 5′ end (head) of the first gene being adjacent to the 3′ end (tail) of the upstream gene. Expression cassettes encoded on the same engineered nucleic acid can be oriented in an opposite direction, i.e., transcription of separate cassettes proceeds in the opposite direction (also referred to herein as “bidirectional”). Expression cassettes encoded on the same engineered nucleic acid oriented in opposite directions can be oriented in a “head-to-head” directionality. As used herein, head-to-head refers to the 5′ end (head) of a first gene of a bidirectional construct being adjacent to the 5′ end (head) of an upstream gene of the bidirectional construct. Expression cassettes encoded on the same engineered nucleic acid oriented in opposite directions can be oriented in a “tail-to-tail” directionality. As used herein, tail-to-tail refers to the 3′ end (tail) of a first gene of a bidirectional construct being adjacent to the 3′ end (tail) of an upstream gene of the bidirectional construct. For example, and without limitation, FIG. 1 schematically depicts a cytokine-CAR bidirectional construct in head-to-head directionality (FIG. 1A), head-to-tail directionality (FIG. 1B), and tail-to-tail directionality (FIG. 1C).

“Linkers,” as used herein can refer to polypeptides that link a first polypeptide sequence and a second polypeptide sequence, the multicistronic linkers described above, or the additional promoters that are operably linked to additional ORFs described above.

Exogenous polynucleotide sequences encoded by the expression cassette can include a 3′untranslated region (UTR) comprising an mRNA-destabilizing element that is operably linked to the exogenous polynucleotide sequence, such as exogenous polynucleotide sequences encoding a cytokine (e.g., IL12 or IL12p70). In some embodiments, the mRNA-destabilizing element comprises an AU-rich element and/or a stem-loop destabilizing element (SLDE). In some embodiments, the mRNA-destabilizing element comprises an AU-rich element. In some embodiments, the AU-rich element includes at least two overlapping motifs of the sequence ATTTA (SEQ ID NO: 209). In some embodiments, the AU-rich element comprises ATTTATTTATTTATTTATTTA (SEQ ID NO: 210). In some embodiments, the mRNA-destabilizing element comprises a stem-loop destabilizing element (SLDE). In some embodiments, the SLDE comprises CTGTTTAATATTTAAACAG (SEQ ID NO: 211). In some embodiments, the mRNA-destabilizing element comprises at least one AU-rich element and at least one SLDE. “AuSLDE” as used herein refers to an AU-rich element operably linked to a stem-loop destabilizing element (SLDE). An exemplary AuSLDE sequence comprises ATTTATTTATTTATTTATTTAacatcggttccCTGTTTAATATTTAAACAG (SEQ ID NO: 212). In some embodiments, the mRNA-destabilizing element comprises a 2× AuSLDE. An exemplary AuSLDE sequence is provided as ATTTATTTATTTATTTATTTAacatcggttccCTGTTTAATATTTAAACAGtgcggtaagcATTTA TTTATTTATTTATTTAacatcggttccCTGTTTAATATTTAAACAG (SEQ ID NO: 213).

In certain embodiments, an engineered nucleic acid described herein comprises an insulator sequence. Such insulator sequences function to prevent inappropriate interactions between adjacent regions of a construct. In certain embodiments, an insulator sequence comprises the nucleic acid sequence of ACAATGGCTGGCCCATAGTAAATGCCGTGTTAGTGTGTTAGTTGCTGTTCTTCCACG TCAGAAGAGGCACAGACAAATTACCACCAGGTGGCGCTCAGAGTCTGCGGAGGCAT CACAACAGCCCTGAATTTGAATCCTGCTCTGCCACTGCCTAGTTGAGACCTTTTACT ACCTGACTAGCTGAGACATTTACGACATTTACTGGCTCTAGGACTCATTTTATTCAT TTCATTACTTTTTTTTTCTTTGAGACGGAATCTCGCTCT (SEQ ID NO: 300). In certain embodiments, an insulator sequence comprises a sequence that is at least 90%, at least 91%, at least 92%, at least 93%, at least 94%, at least 95%, at least 96%, at least 97%, at least 98%, or at least 99% identical to SEQ ID NO: 300.

### Engineered Cells

Provided herein are engineered immunoresponsive cells, and methods of producing the engineered immunoresponsive cells, that produce a protein described herein (e.g., a cytokine, CAR, ACP, and/or membrane-cleavable chimeric protein described herein). In general, engineered immunoresponsive cells of the present disclosure may be engineered to express the proteins provided for herein, such as a cytokine, CAR, ACP, and/or the membrane-cleavable chimeric proteins having the formula S-C-MT or MT-C-S described herein. For example, immunoresponsive cells may be engineered to express a cytokine, CAR, and membrane-cleavable chimeric protein described herein. For example, immunoresponsive cells may be engineered to express a cytokine, an aCAR, and membrane-cleavable chimeric protein described herein. For example, immunoresponsive cells may be engineered to express a cytokine, an aCAR, an iCAR, and membrane-cleavable chimeric protein described herein. These cells are referred to herein as “engineered cells.” These cells, which typically contain engineered nucleic acid, do not occur in nature. In some embodiments, the cells are engineered to include a nucleic acid comprising a promoter operably linked to a nucleotide sequence encoding a protein, for example, a cytokine, CAR, ACP, and/or a membrane-cleavable chimeric protein. An engineered cell can comprise an engineered nucleic acid integrated into the cell's genome. An engineered cell can comprise an engineered nucleic acid capable of expression without integrating into the cell's genome, for example, engineered with a transient expression system such as a plasmid or mRNA.

The present disclosure also encompasses additivity and synergy between a protein(s) and the engineered cell from which they are produced. In some embodiments, cells are engineered to produce at least two (e.g., 2, 3, 4, 5, 6, 7, 8, 9, 10 or more) proteins, for example at least each of a cytokine, CAR, ACP, and membrane-cleavable chimeric protein. In some embodiments, cells are engineered to produce a cytokine, CAR, and membrane-cleavable chimeric protein described herein. In some embodiments, cells are engineered to produce a cytokine, an aCAR, and membrane-cleavable chimeric protein described herein. In some embodiments, cells are engineered to produce a cytokine, an aCAR, an iCAR, and membrane-cleavable chimeric protein described herein. In general, immunoresponsive cells provide herein are engineered to produce at least one membrane-cleavable chimeric protein having a cytokine effector molecule that is not natively produced by the cells, a CAR, and an ACP. In general, immunoresponsive cells provided herein are engineered to produce at least two cytokines, at least one of which is a membrane-cleavable chimeric protein having a cytokine effector molecule, a CAR, and an ACP. In some embodiments, immunoresponsive cells provided herein are engineered to produce at least two cytokines, at least one of which is a membrane-cleavable chimeric protein having a cytokine effector molecule, and a CAR. In some embodiments, immunoresponsive cells provided herein are engineered to produce at least two cytokines, at least one of which is a membrane-cleavable chimeric protein having a cytokine effector molecule, and two CARs. In some embodiments, immunoresponsive cells provided herein are engineered to produce at least two cytokines, at least one of which is a membrane-cleavable chimeric protein having a cytokine effector molecule, an aCAR, and an iCAR. In some embodiments, the aCAR Such an effector molecule may, for example, complement the function of effector molecules natively produced by the cells.

In some embodiments, a cell (e.g., an immune cell) is engineered to produce multiple proteins. For example, cells may be engineered to produce 2-20 different proteins, such as 2-20 different membrane-cleavable proteins. In some embodiments, a cell (e.g., an immunoresponsive cell) is engineered to produce at least 4 distinct proteins exogenous to the cell. In some embodiments, a cell (e.g., an immunoresponsive cell) is engineered to produce 4 distinct proteins exogenous to the cell. In some embodiments, cells engineered to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11,7-10, 7-9, 7-8, 8-20, 8-19, 8-18, 8-17, 8-16, 8-15, 8-14, 8-13, 8-12, 8-11, 8-10, 8-9, 9-20,9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 proteins. In some embodiments, cells are engineered to produce 1, 2, 3, 4, 5, 6, 7, 8, 9, 10, 11, 12, 13, 14, 15, 16, 17, 18, 19, or 20 proteins.

In some embodiments, engineered cells comprise one or more engineered nucleic acids encoding a promoter operably linked to a nucleotide sequence encoding a protein (e.g., an expression cassette). In some embodiments, cells are engineered to include a plurality of engineered nucleic acids, e.g., at least two engineered nucleic acids, each encoding a promoter operably linked to a nucleotide sequence encoding at least one (e.g., 1, 2 or 3) protein. For example, cells may be engineered to comprise at least 2, at least 3, at least 4, at least 5, at least 6, at least 7, at least 8, at least 8, at least 9, or at least 10, engineered nucleic acids, each encoding a promoter operably linked to a nucleotide sequence encoding at least one (e.g., 1, 2 or 3) protein. In some embodiments, the cells are engineered to comprise 2, 3, 4, 5, 6, 7, 8, 9, 10, or more engineered nucleic acids, each encoding a promoter operably linked to a nucleotide sequence encoding at least one (e.g., 1, 2 or 3) protein. Engineered cells can comprise an engineered nucleic acid encoding at least one of the linkers described above, such as polypeptides that link a first polypeptide sequence and a second polypeptide sequence, one or more multicistronic linker described above, one or more additional promoters operably linked to additional ORFs, or a combination thereof.

In some embodiments, a cell (e.g., an immune cell) is engineered to express a protease. In some embodiments, a cell is engineered to express a protease heterologous to a cell. In some embodiments, a cell is engineered to express a protease heterologous to a cell expressing a protein, such as a heterologous protease that cleaves the protease cleavage site of a membrane-cleavable chimeric protein. In some embodiments, engineered cells comprise one or more engineered nucleic acids encoding a promoter operably linked to a nucleotide sequence encoding a protease, such as a heterologous protease. Protease and protease cleavage sites are described in greater detail in the Section herein titled “Protease Cleavage site.” In other embodiments, a cell is not engineered to express a heterologous protease that cleaves the protease cleavage site of a membrane-cleavable chimeric protein. In such embodiments, the cell endogenously expresses a protease that cleaves the protease cleavage site of a membrane-cleavable chimeric protein.

Also provided herein are engineered cells that are engineered to produce multiple proteins, at least two of which include effector molecules that modulate different tumor-mediated immunosuppressive mechanisms. In some embodiments, at least one (e.g., 1, 2, 3, 4, 5, or more) protein includes an effector molecule that stimulates at least one immunostimulatory mechanism in the tumor microenvironment, or inhibits at least one immunosuppressive mechanism in the tumor microenvironment. In some embodiments, at least one (e.g., 1, 2, 3, 4, 5, or more) protein includes an effector molecule that inhibits at least one immunosuppressive mechanism in the tumor microenvironment, and at least one protein (e.g., 1, 2, 3, 4, 5, or more) inhibits at least one immunosuppressive mechanism in the tumor microenvironment. In yet other embodiments, at least two (e.g., 2, 3, 4, 5, or more) of the proteins are effector molecules that each stimulate at least one immunostimulatory mechanism in the tumor microenvironment. In still other embodiments, at least two (e.g., 1, 2, 3, 4, 5, or more) of the proteins are effector molecules that each inhibit at least one immunosuppressive mechanism in the tumor microenvironment.

In some embodiments, a cell (e.g., an immune cell) is engineered to produce at least one protein including an effector molecule that stimulates T cell or NK cell signaling, activity and/or recruitment. In some embodiments, a cell is engineered to produce at least one protein that includes an effector molecule that stimulates antigen presentation and/or processing. In some embodiments, a cell is engineered to produce at least one protein that includes an effector molecule that stimulates natural killer cell-mediated cytotoxic signaling, activity and/or recruitment. In some embodiments, a cell is engineered to produce at least one protein that includes an effector molecule that stimulates dendritic cell differentiation and/or maturation. In some embodiments, a cell is engineered to produce at least one protein that includes an effector molecule that stimulates immune cell recruitment. In some embodiments, a cell is engineered to produce at least one protein includes an effector molecule that that stimulates M1 macrophage signaling, activity and/or recruitment. In some embodiments, a cell is engineered to produce at least one protein that includes an effector molecule that stimulates Th1 polarization. In some embodiments, a cell is engineered to produce at least one protein that includes an effector molecule that stimulates stroma degradation. In some embodiments, a cell is engineered to produce at least one protein that includes an effector molecule that stimulates immunostimulatory metabolite production. In some embodiments, a cell is engineered to produce at least one protein that includes an effector molecule that stimulates Type I interferon signaling. In some embodiments, a cell is engineered to produce at least one protein that includes an effector molecule that inhibits negative costimulatory signaling. In some embodiments, a cell is engineered to produce at least one protein that includes an effector molecule that inhibits pro-apoptotic signaling (e.g., via TRAIL) of anti-tumor immune cells. In some embodiments, a cell is engineered to produce at least one protein that includes an effector molecule that inhibits T regulatory (T_{reg}) cell signaling, activity and/or recruitment. In some embodiments, a cell is engineered to produce at least one protein that includes an effector molecule that inhibits tumor checkpoint molecules. In some embodiments, a cell is engineered to produce at least one protein that includes an effector molecule that activates stimulator of interferon genes (STING) signaling. In some embodiments, a cell is engineered to produce at least one protein that includes an effector molecule that inhibits myeloid-derived suppressor cell signaling, activity and/or recruitment. In some embodiments, a cell is engineered to produce at least one protein that includes an effector molecule that degrades immunosuppressive factors/metabolites. In some embodiments, a cell is engineered to produce at least one protein that includes an effector molecule that inhibits vascular endothelial growth factor signaling. In some embodiments, a cell is engineered to produce at least one protein that includes an effector molecule that directly kills tumor cells (e.g., granzyme, perform, oncolytic viruses, cytolytic peptides and enzymes, anti-tumor antibodies, e.g., that trigger ADCC).

In some embodiments, at least one protein including an effector molecule that: stimulates T cell signaling, activity and/or recruitment, stimulates antigen presentation and/or processing, stimulates natural killer cell-mediated cytotoxic signaling, activity and/or recruitment, stimulates dendritic cell differentiation and/or maturation, stimulates immune cell recruitment, stimulates macrophage signaling, stimulates stroma degradation, stimulates immunostimulatory metabolite production, or stimulates Type I interferon signaling; and at least one protein including an effector molecule that inhibits negative costimulatory signaling, inhibits pro-apoptotic signaling of anti-tumor immune cells, inhibits T regulatory (T_{reg}) cell signaling, activity and/or recruitment, inhibits tumor checkpoint molecules, activates stimulator of interferon genes (STING) signaling, inhibits myeloid-derived suppressor cell signaling, activity and/or recruitment, degrades immunosuppressive factors/metabolites, inhibits vascular endothelial growth factor signaling, or directly kills tumor cells.

In some embodiments, an immunoresponsive cell is engineered to produce at least one effector molecule cytokine selected from IL7, IL15, IL12, an IL12p70 fusion protein, IL18, and IL21. In some embodiments, an immunoresponsive cell is engineered to produce at least two effector molecule cytokines selected from IL7, IL15, IL12, an IL12p70 fusion protein, IL18, and IL21. In some embodiments, an immunoresponsive cell is engineered to produce at least two effector molecule cytokines selected from IL7, IL15, IL12, an IL12p70 fusion protein, IL18, and IL21. In some embodiments, an immunoresponsive cell is engineered to produce at least the effector molecule cytokines IL15 and IL12p70 fusion protein. In some embodiments, an immunoresponsive cell is engineered to produce at least one membrane-cleavable chimeric protein including an effector molecule cytokine selected from IL15, IL12, an IL12p70 fusion protein, IL18, and IL21. In some embodiments, an immunoresponsive cell is engineered to produce at least two membrane-cleavable chimeric protein including effector molecule cytokines selected from IL15, IL12, an IL12p70 fusion protein, IL18, and IL21. In some embodiments, an immunoresponsive cell is engineered to produce at least one membrane-cleavable chimeric protein including an effector molecule cytokine selected from IL7, IL15, IL12, an IL12p70 fusion protein, IL18, and IL21 and an additional effector molecule cytokine selected from IL7, IL15, IL12, an IL12p70 fusion protein, IL18, and IL21. In particular embodiments, an immunoresponsive cell is engineered to produce two cytokines, IL15 and IL21. In particular embodiments, at least one of the two cytokines is a membrane cleavable chimeric protein.

In certain embodiments, the IL15 comprises the amino acid sequence of NWVNVISDLKKIEDLIQSMHIDATLYTESDVHPSCKVTAMKCFLLELQVISLESGDASIH DTVENLIILANNSLSSNGNVTESGCKECEELEEKNIKEFLQSFVHIVQMFINTS (SEQ ID NO: 285). An exemplary nucleic acid sequence encoding SEQ ID NO: 285 is AATTGGGTCAACGTGATCAGCGACCTGAAGAAGATCGAGGACCTGATCCAGAGCAT GCACATCGACGCCACACTGTACACCGAGAGCGACGTGCACCCTAGCTGTAAAGTGA CCGCCATGAAGTGCTTTCTGCTGGAACTGCAAGTGATCAGCCTGGAAAGCGGCGAC GCCAGCATCCACGACACCGTGGAAAACCTGATCATCCTGGCCAACAACAGCCTGAG CAGCAACGGCAATGTGACCGAGTCCGGCTGCAAAGAGTGCGAGGAACTGGAAGAG AAGAATATCAAAGAGTTCCTGCAGAGCTTCGTGCACATCGTGCAGATGTTCATCAA CACAAGC (SEQ ID NO: 286). In certain embodiments, a nucleic acid encoding SEQ ID NO: 285 comprises a sequence that is at least 90%, at least 91%, at least 92%, at least 93%, at least 94%, at least 95%, at least 96%, at least 97%, at least 98%, or at least 99% identical to SEQ ID NO: 286. In certain embodiments, the IL15 comprises an mIgGKVII leader sequence. In certain embodiments, the IL15 comprises the amino acid sequence of METDTLLLWVLLLWVPGSTGNWVNVISDLKKIEDLIQSMHIDATLYTESDVHPSCKVT AMKCFLLELQVISLESGDASIHDTVENLIILANNSLSSNGNVTESGCKECEELEEKNIKEF LQSFVHIVQMFINTS (SEQ ID NO: 357). An exemplary nucleic acid sequence encoding SEQ ID NO: 357 is ATGGAAACCGACACACTGCTGCTGTGGGTGCTGCTTCTTTGGGTGCCCGGCTCTACA GGCAACTGGGTCAACGTGATCAGCGACCTGAAGAAGATCGAGGACCTGATCCAGA GCATGCACATCGACGCCACACTGTACACCGAGAGCGACGTGCACCCTAGCTGTAAA GTGACCGCCATGAAGTGCTTTCTGCTGGAACTGCAAGTGATCAGCCTGGAAAGCGG CGACGCCAGCATCCACGACACCGTGGAAAACCTGATCATCCTGGCTAACAACAGCC TGAGCAGCAACGGCAATGTGACCGAGTCCGGCTGCAAAGAGTGCGAGGAACTGGA AGAGAAGAATATCAAAGAGTTCCTCCAGAGCTTCGTGCACATCGTGCAGATGTTCA TCAACACCAGC (SEQ ID NO: 369). In certain embodiments, a nucleic acid encoding SEQ ID NO: 357 comprises a sequence that is at least 90%, at least 91%, at least 92%, at least 93%, at least 94%, at least 95%, at least 96%, at least 97%, at least 98%, or at least 99% identical to SEQ ID NO: 369.

As used herein, the terms “membrane-cleavable,” “controlled release,” and “calibrated release” are used interchangeably. In certain embodiments, the IL15 is membrane cleavable. In certain embodiments, the IL15 is controlled release IL15 (crIL15). In certain embodiments, the crIL15 comprises a B7-1 transmembrane domain. In certain embodiments, the B7-1 transmembrane domain comprises the amino acid sequence of SEQ ID NO 219. In certain embodiments, the crIL15 comprises a “slow” protease cleavage site comprising the amino acid sequence of VTPEPIFSLI (SEQ ID NO: 191). In certain embodiments, the crIL15 comprising the “slow” protease cleavage site comprises the amino acid sequence of

(SEQ ID NO: 355)
MDWTWILFLVAAATRVHSYPYDVPDYAGGGGSNWVNVISDLKKIEDLIQ
SMHIDATLYTESDVHPSCKVTAMKCFLLELQVISLESGDASIHDTVENL
IILANNSLSSNGNVTESGCKECEELEEKNIKEFLQSFVHIVQMFINTSS
GGGGSGGGGSGVTPEPIFSLIGGGSGGGGSGGGSLQLLPSWAITLISVN
GIFVICCLTYCFAPRCRERRRNERLRRESVRPV.

An exemplary nucleic acid sequence encoding SEQ ID NO: 355 is

(SEQ ID NO: 367)
ATGGACTGGACTTGGATACTCTTTCTGGTCGCTGCCGCCACACGGGTGC
ACTCTTATCCATATGATGTTCCAGATTATGCTGGCGGAGGCGGTTCTAA
TTGGGTCAACGTGATCAGCGACCTGAAGAAGATCGAGGACCTGATCCAG
AGCATGCACATCGACGCCACACTGTACACCGAGTCCGATGTGCACCCTA
GCTGCAAAGTGACCGCCATGAAGTGCTTTCTGCTGGAACTGCAAGTGAT
CAGCCTGGAAAGCGGCGACGCCAGCATCCACGATACCGTGGAAAATCTG
ATCATCCTGGCCAACAACAGCCTGTCCAGCAACGGCAATGTGACCGAGA
GCGGCTGCAAAGAGTGCGAGGAACTGGAAGAGAAGAACATCAAAGAGTT
TCTGCAGAGCTTCGTCCACATCGTGCAGATGTTCATCAACACCTCATCA
GGCGGCGGTGGTAGTGGAGGCGGAGGCTCAGGCGTGACCCCTGAGCCTA
TCTTCAGCCTGATCGGCGGAGGTTCCGGAGGTGGCGGTTCCGGCGGAGG
ATCTCTTCAATTGCTGCCTAGCTGGGCCATCACACTGATCTCCGTGAAC
GGCATCTTCGTGATCTGCTGCCTGACCTACTGCTTCGCCCCTAGATGCA
GAGAGCGGAGAAGAAACGAGCGGCTGAGAAGAGAAAGCGTGCGGCCTGT
G.

In some embodiments, the crIL15 comprising the “slow” protease cleavage site also comprises a furin cleavage site. The crIL15 comprising the “slow” protease cleavage site and the furin cleavage site may comprise the amino acid sequence MDWTWILFLVAAATRVHSYPYDVPDYAGGGGSNWVNVISDLKKIEDLIQSMHIDATLY TESDVHPSCKVTAMKCFLLELQVISLESGDASIHDTVENLIILANNSLSSNGNVTESGCKE CEELEEKNIKEFLQSFVHIVQMFINTSSGGGGSGGGGSGVTPEPIFSLIGGGSGGGGSGGG SLQLLPSWAITLISVNGIFVICCLTYCFAPRCRERRRNERLRRESVRPVRRKR (SEQ ID NO: 415). An exemplary nucleic acid sequence encoding SEQ ID NO: 415 is ATGGACTGGACTTGGATACTCTTTCTGGTCGCTGCCGCCACACGGGTGCACTCTTAT CCATATGATGTTCCAGATTATGCTGGCGGAGGCGGTTCTAATTGGGTCAACGTGATC AGCGACCTGAAGAAGATCGAGGACCTGATCCAGAGCATGCACATCGACGCCACACT GTACACCGAGTCCGATGTGCACCCTAGCTGCAAAGTGACCGCCATGAAGTGCTTTCT GCTGGAACTGCAAGTGATCAGCCTGGAAAGCGGCGACGCCAGCATCCACGATACCG TGGAAAATCTGATCATCCTGGCCAACAACAGCCTGTCCAGCAACGGCAATGTGACC GAGAGCGGCTGCAAAGAGTGCGAGGAACTGGAAGAGAAGAACATCAAAGAGTTTC TGCAGAGCTTCGTCCACATCGTGCAGATGTTCATCAACACCTCATCAGGCGGCGGTG GTAGTGGAGGCGGAGGCTCAGGCGTGACCCCTGAGCCTATCTTCAGCCTGATCGGC GGAGGTTCCGGAGGTGGCGGTTCCGGCGGAGGATCTCTTCAATTGCTGCCTAGCTG GGCCATCACACTGATCTCCGTGAACGGCATCTTCGTGATCTGCTGCCTGACCTACTG CTTCGCCCCTAGATGCAGAGAGCGGAGAAGAAACGAGCGGCTGAGAAGAGAAAGC GTGCGGCCTGTGAGAAGAAAACGC (SEQ ID NO: 416). In certain embodiments, a nucleic acid encoding SEQ ID NO: 355 comprises a sequence that is at least 90%, at least 91%, at least 92%, at least 93%, at least 94%, at least 95%, at least 96%, at least 97%, at least 98%, or at least 99% identical to SEQ ID NO: 367. In certain embodiments, the crIL15 comprises a “fast” protease cleavage site comprising the amino acid sequence of PRAEALKGG (SEQ ID NO: 180). In certain embodiments, the crIL15 comprising the “fast” protease cleavage site comprises the amino acid sequence of MDWTWILFLVAAATRVHSYPYDVPDYAGGGGSNWVNVISDLKKIEDLIQSMHIDATLY TESDVHPSCKVTAMKCFLLELQVISLESGDASIHDTVENLIILANNSLSSNGNVTESGCKE CEELEEKNIKEFLQSFVHIVQMFINTSPRAEALKGGSGGGGSGGGGSGGGGSGGGGSGG GSLQLLPSWAITLISVNGIFVICCLTYCFAPRCRERRRNERLRRESVRPV (SEQ ID NO: 356). An exemplary nucleic acid sequence encoding SEQ ID NO: 356 is ATGGACTGGACTTGGATACTCTTTCTGGTCGCTGCCGCCACACGGGTGCACTCTTAT CCATATGATGTTCCAGATTATGCTGGCGGAGGCGGTTCTAATTGGGTCAACGTGATC AGCGACCTGAAGAAGATCGAGGACCTGATCCAGAGCATGCACATCGACGCCACACT GTACACCGAGTCCGATGTGCACCCTAGCTGCAAAGTGACCGCCATGAAGTGCTTTCT GCTGGAACTGCAAGTGATCAGCCTGGAAAGCGGCGACGCCAGCATCCACGATACCG TGGAAAATCTGATCATCCTGGCCAACAACAGCCTGTCCAGCAACGGCAATGTGACC GAGAGCGGCTGCAAAGAGTGCGAGGAACTGGAAGAGAAGAACATCAAAGAGTTTC TGCAGAGCTTCGTCCACATCGTGCAGATGTTCATCAACACCTCACCCAGAGCCGAG GCTCTGAAAGGCGGATCAGGCGGCGGTGGTAGTGGAGGCGGAGGCTCAGGCGGCG GAGGTTCCGGAGGTGGCGGTTCCGGCGGAGGATCTCTTCAATTGCTGCCTAGCTGG GCCATCACACTGATCTCCGTGAACGGCATCTTCGTGATCTGCTGCCTGACCTACTGC TTCGCCCCTAGATGCAGAGAGCGGAGAAGAAACGAGCGGCTGAGAAGAGAAAGCG TGCGGCCTGTG (SEQ ID NO: 368). In certain embodiments, a nucleic acid encoding SEQ ID NO: 356 comprises a sequence that is at least 90%, at least 91%, at least 92%, at least 93%, at least 94%, at least 95%, at least 96%, at least 97%, at least 98%, or at least 99% identical to SEQ ID NO: 368.

In certain embodiments, the crIL15 comprises the amino acid sequence of MDWTWILFLVAAATRVHSNWVNVISDLKKIEDLIQSMHIDATLYTESDVHPSCKVTAM KCFLLELQVISLESGDASIHDTVENLIILANNSLSSNGNVTESGCKECEELEEKNIKEFLQS FVHIVQMFINTS (SEQ ID NO: 410). An exemplary nucleic acid sequence encoding SEQ ID NO: 410 is

(SEQ ID NO: 411)
ATGGACTGGACTTGGATACTCTTTCTGGTCGCTGCCGCCACACGGGTGCA
CTCTAATTGGGTCAACGTGATCAGCGACCTGAAGAAGATCGAGGACCTGA
TCCAGAGCATGCACATCGACGCCACACTGTACACCGAGTCCGATGTGCAC
CCTAGCTGCAAAGTGACCGCCATGAAGTGCTTTCTGCTGGAACTGCAAGT
GATCAGCCTGGAAAGCGGCGACGCCAGCATCCACGATACCGTGGAAAATC
TGATCATCCTGGCCAACAACAGCCTGTCCAGCAACGGCAATGTGACCGAG
AGCGGCTGCAAAGAGTGCGAGGAACTGGAAGAGAAGAACATCAAAGAGTT
TCTGCAGAGCTTCGTCCACATCGTGCAGATGTTCATCAACACCTCA.

In certain embodiments, the crIL15 comprises a sushi domain. In certain embodiments the crIL15 comprises an IgE leader sequence. In certain embodiments, the crIL15 comprises a sushi domain and an IgE leader sequence. In certain embodiments, the crIL15 comprises the amino acid sequence of MDWTWILFLVAAATRVHSITCPPPMSVEHADIWVKSYSLYSRERYICNSGFKRKAGTSS LTECVLNKATNVAHWTTPSLKCIRSGGSGGGGSGGGSGGGGSLQNWVNVISDLKKIED LIQSMHIDATLYTESDVHPSCKVTAMKCFLLELQVISLESGDASIHDTVENLIILANDSLS SNGNVTESGCKECEELEEKNIKEFLQSFVHIVQMFINTSSGGGGSGGGGSGVTPEPIFSLI GGGSGGGGSGGGSLQLLPSWAITLISVNGIFVICCLTYCFAPRCRERRRNERLRRESVRP V (SEQ ID NO: 361). An exemplary nucleic acid sequence encoding SEQ ID NO: 361 is AACGGGCCGCACAGATTCTCTTCTCAGCCGTTCGTTTCTCCGCCGCTCTCTGCATCTA GGGGCGAAGCAGTAGGTCAGGCAGCAGATCACGAAGATGCCGTTCACGGAGATCA GTGTGATGGCCCAGCTAGGCAGCAGTTGCAGAGATCCGCCACCACTTCCTCCGCCTC CGCTACCGCCTCCGATCAGGCTGAAGATAGGCTCGGGTGTAACTCCGCTTCCACCTC CGCCAGATCCTCCGCCGCCAGAGCTTGTGTTGATGAACATCTGCACGATGTGCACG AAGCTCTGCAGGAACTCTTTGATATTCTTCTCTTCCAGTTCCTCGCACTCTTTGCAGC CGGACTCGGTCACATTGCCGTTGCTGCTCAGGCTGTCGTTGGCCAGGATGATCAGGT TTTCCACGGTGTCGTGGATGCTGGCGTCGCCGCTTTCCAGGCTGATCACTTGCAGTT CCAGCAGAAAGCACTTCATGGCGGTCACTTTACAGCTAGGGTGCACGTCGCTCTCG GTGTACAGTGTGGCGTCGATGTGCATGCTCTGGATCAGGTCCTCGATCTTCTTCAGG TCGCTGATCACGTTGACCCAATTCTGCAGAGATCCTCCGCCTCCGCTTCCACCGCCA GAACCTCCGCCGCCAGATCCGCCGCTTCTGATACACTTCAGGCTAGGTGTGGTCCAG TGGGCCACATTGGTGGCCTTGTTCAGCACACACTCGGTCAGGCTGCTGGTGCCGGCC TTTCTCTTGAAGCCGCTGTTGCAGATGTACCGCTCTCTGCTGTACAGGCTGTAGCTCT TGACCCAGATGTCGGCGTGTTCCACGCTCATAGGTGGAGGACAGGTGATGCTGTGC ACTCTTGTGGCAGCGGCCACCAGAAACAGGATCCAGGTCCAGTCCAT (SEQ ID NO: 372). In certain embodiments, a nucleic acid encoding SEQ ID NO: 361 comprises a sequence that is at least 90%, at least 91%, at least 92%, at least 93%, at least 94%, at least 95%, at least 96%, at least 97%, at least 98%, or at least 99% identical to SEQ ID NO: 372.

In certain embodiments, the chimeric IL15 comprises a sushi domain. In certain embodiments the chimeric IL15 comprises an IgE leader sequence. In certain embodiments, the chimeric IL15 comprises a sushi domain and an IgE leader sequence. In certain embodiments, the chimeric IL15 comprises the amino acid sequence of MDWTWILFLVAAATRVHSNWVNVISDLKKIEDLIQSMHIDATLYTESDVHPSCKVTAM KCFLLELQVISLESGDASIHDTVENLIILANNSLSSNGNVTESGCKECEELEEKNIKEFLQS FVHIVQMFINTSSGGSGGGGSGGGSGGGGSLQITCPPPMSVEHADIWVKSYSLYSRERYI CNSGFKRKAGTSSLTECVLNKATNVAHWTTPSLKCIRAAAIEVMYPPPYLDNEKSNGTII HVKGKHLCPSPLFPGPSKPFWVLVVVGGVLACYSLLVTVAFIIFWVKSRQTPPLASVEM EAMEALPVTWGTSSRDEDLENCSHHL (SEQ ID NO: 391). An exemplary nucleic acid sequence encoding SEQ ID NO: 391 is ATGGACTGGACTTGGATACTCTTTCTGGTCGCTGCCGCCACACGGGTGCACTCTAAT TGGGTCAACGTGATCAGCGACCTGAAGAAGATCGAGGACCTGATCCAGAGCATGCA CATCGACGCCACACTGTACACCGAGAGCGACGTGCACCCTAGCTGTAAAGTGACCG CCATGAAGTGCTTTCTGCTGGAACTGCAAGTGATCAGCCTGGAAAGCGGCGACGCC AGCATCCACGACACCGTGGAAAACCTGATCATCCTGGCCAACAACAGCCTGAGCAG CAACGGCAATGTGACCGAGTCCGGCTGCAAAGAGTGCGAGGAACTGGAAGAGAAG AATATCAAAGAGTTCCTGCAGAGCTTCGTGCACATCGTGCAGATGTTCATCAACACC AGCAGCGGCGGATCTGGCGGCGGAGGTTCTGGCGGTGGAAGCGGAGGCGGAGGAT CTCTCCAGATCACATGCCCTCCACCTATGAGCGTGGAACACGCCGACATCTGGGTCA AGAGCTACAGCCTGTACAGCAGAGAGCGGTACATCTGCAACAGCGGCTTCAAGAGA AAGGCCGGCACAAGCAGCCTGACCGAGTGCGTGCTGAACAAGGCCACAAATGTGG CCCACTGGACCACACCTAGCCTGAAGTGCATCAGAGCAGCAGCTATCGAGGTGATG TATCCTCCGCCCTACCTGGATAATGAAAAGAGTAATGGGACTATCATTCATGTAAA AGGGAAGCATCTTTGTCCTTCTCCCCTTTTCCCCGGTCCGTCTAAACCTTTCTGGGTG CTCGTGGTTGTTGGCGGAGTGCTGGCCTGTTACTCTCTGCTGGTCACCGTGGCCTTC ATCATCTTTTGGGTCAAGTCCAGACAGACACCTCCTCTGGCCAGCGTGGAAATGGA AGCCATGGAAGCTCTGCCTGTGACCTGGGGCACCAGCTCCAGAGATGAGGACCTGG AAAACTGCTCCCACCACCTGTAA (SEQ ID NO: 392). In certain embodiments, a nucleic acid encoding SEQ ID NO: 391 comprises a sequence that is at least 90%, at least 91%, at least 92%, at least 93%, at least 94%, at least 95%, at least 96%, at least 97%, at least 98%, or at least 99% identical to SEQ ID NO: 392.

In certain embodiments, the IL15 is a membrane-bound IL15 (mbIL15). In certain embodiments, the mbIL15 comprises the amino acid sequence of MDWTWILFLVAAATRVHSYPYDVPDYAGGGGSNWVNVISDLKKIEDLIQSMHIDATLY TESDVHPSCKVTAMKCFLLELQVISLESGDASIHDTVENLIILANNSLSSNGNVTESGCKE CEELEEKNIKEFLQSFVHIVQMFINTSSGGGGSGGGGSGGGGSGGGGSGGGSLQLLPSW AITLISVNGIFVICCLTYCFAPRCRERRRNERLRRESVRPV (SEQ ID NO: 358). An exemplary nucleic acid sequence encoding SEQ ID NO: 358 is ATGGACTGGACTTGGATACTCTTTCTGGTCGCTGCCGCCACACGGGTGCACTCTTAT CCATATGATGTTCCAGATTATGCTGGCGGAGGCGGTTCTAATTGGGTCAACGTGATC AGCGACCTGAAGAAGATCGAGGACCTGATCCAGAGCATGCACATCGACGCCACACT GTACACCGAGTCCGATGTGCACCCTAGCTGCAAAGTGACCGCCATGAAGTGCTTTCT GCTGGAACTGCAAGTGATCAGCCTGGAAAGCGGCGACGCCAGCATCCACGATACCG TGGAAAATCTGATCATCCTGGCCAACAACAGCCTGTCCAGCAACGGCAATGTGACC GAGAGCGGCTGCAAAGAGTGCGAGGAACTGGAAGAGAAGAACATCAAAGAGTTTC TGCAGAGCTTCGTCCACATCGTGCAGATGTTCATCAACACCTCATCAGGCGGCGGTG GTAGTGGAGGCGGAGGCTCAGGCGGCGGAGGTTCCGGAGGTGGCGGTTCCGGCGG AGGATCTCTTCAATTGCTGCCTAGCTGGGCCATCACACTGATCTCCGTGAACGGCAT CTTCGTGATCTGCTGCCTGACCTACTGCTTCGCCCCTAGATGCAGAGAGCGGAGAAG AAACGAGCGGCTGAGAAGAGAAAGCGTGCGGCCTGTG (SEQ ID NO: 370). In certain embodiments, a nucleic acid encoding SEQ ID NO: 358 comprises a sequence that is at least 90%, at least 91%, at least 92%, at least 93%, at least 94%, at least 95%, at least 96%, at least 97%, at least 98%, or at least 99% identical to SEQ ID NO: 370.

In certain embodiments, the IL21 comprises the amino acid sequence of QGQDRHMIRMRQLIDIVDQLKNYVNDLVPEFLPAPEDVETNCEWSAFSCFQKAQLKSA NTGNNERIINVSIKKLKRKPPSTNAGRRQKHRLTCPSCDSYEKKPPKEFLERFKSLLQKM IHQHLSSRTHGSEDS (SEQ ID NO: 360) An exemplary nucleic acid sequence encoding SEQ ID NO: 360 is CAGGGCCAAGACCGGCACATGATCCGGATGAGACAGCTGATCGACATCGTGGACCA GCTGAAGAACTACGTGAACGACCTGGTGCCTGAGTTTCTGCCCGCTCCTGAGGACG TGGAAACAAACTGCGAGTGGAGCGCCTTCAGCTGCTTCCAGAAGGCCCAGCTGAAA AGCGCCAACACCGGCAACAACGAGCGGATCATCAACGTGTCCATCAAGAAGCTGA AGCGGAAGCCTCCTAGCACCAACGCCGGAAGAAGGCAGAAGCACAGACTGACCTG TCCTAGCTGCGACAGCTACGAGAAGAAGCCTCCAAAAGAGTTTCTCGAGCGGTTCA AGAGCCTGCTGCAGAAGATGATCCACCAGCACCTGTCCAGCAGGACACACGGCAGC GAGGATTCT (SEQ ID NO: 386). In certain embodiments, a nucleic acid encoding SEQ ID NO: 360 comprises a sequence that is at least 90%, at least 91%, at least 92%, at least 93%, at least 94%, at least 95%, at least 96%, at least 97%, at least 98%, or at least 99% identical to SEQ ID NO: 386.

In certain embodiments, the IL21 comprises a codon optimized IL21 leader sequence. In certain embodiments, the IL21 comprises the amino acid sequence of MERIVICLMVIFLGTLVHKSSSQGQDRHMIRMRQLIDIVDQLKNYVNDLVPEFLPAPED VETNCEWSAFSCFQKAQLKSANTGNNERIINVSIKKLKRKPPSTNAGRRQKHRLTCPSC DSYEKKPPKEFLERFKSLLQKMIHQHLSSRTHGSEDS (SEQ ID NO: 359) An exemplary nucleic acid sequence encoding SEQ ID NO: 359 is ATGGAACGGATCGTGATCTGCCTGATGGTCATCTTCCTGGGCACCCTGGTGCACAAG AGCAGCTCTCAGGGCCAAGACCGGCACATGATCCGGATGAGACAGCTGATCGACAT CGTGGACCAGCTGAAGAACTACGTGAACGACCTGGTGCCTGAGTTTCTGCCCGCTC CTGAGGACGTGGAAACAAACTGCGAGTGGAGCGCCTTCAGCTGCTTCCAGAAGGCC CAGCTGAAAAGCGCCAACACCGGCAACAACGAGCGGATCATCAACGTGTCCATCAA GAAGCTGAAGCGGAAGCCTCCTAGCACCAACGCCGGAAGAAGGCAGAAGCACAGA CTGACCTGTCCTAGCTGCGACAGCTACGAGAAGAAGCCTCCAAAAGAGTTTCTCGA GCGGTTCAAGAGCCTGCTGCAGAAGATGATCCACCAGCACCTGTCCAGCAGGACAC ACGGCAGCGAGGATTCT (SEQ ID NO: 371). In certain embodiments, a nucleic acid encoding SEQ ID NO: 359 comprises a sequence that is at least 90%, at least 91%, at least 92%, at least 93%, at least 94%, at least 95%, at least 96%, at least 97%, at least 98%, or at least 99% identical to SEQ ID NO: 371. Another exemplary nucleic acid sequence encoding SEQ ID NO: 359 is ATGGAACGGATCGTGATCTGCCTGATGGTCATCTTCCTGGGCACCCTGGTGCACAAG AGCAGCTCTCAGGGCCAAGACCGGCACATGATCCGGATGAGACAGCTGATCGACAT CGTGGACCAGCTGAAGAACTACGTGAACGACCTGGTGCCTGAGTTCCTGCCTGCTC CTGAGGACGTGGAAACAAACTGCGAGTGGAGCGCCTTCAGCTGCTTCCAGAAGGCC CAGCTGAAAAGCGCCAACACCGGCAACAACGAGCGGATCATCAACGTGTCCATCAA GAAGCTGAAGCGGAAGCCTCCTAGCACCAACGCCGGAAGAAGGCAGAAGCACAGA CTGACCTGTCCTAGCTGCGACAGCTACGAGAAGAAGCCTCCAAAAGAGTTCCTGGA ACGGTTCAAGAGCCTGCTGCAGAAGATGATCCACCAGCACCTGAGCAGCAGAACCC ACGGCAGCGAGGACTCC (SEQ ID NO: 412). In some embodiments, the IL21 comprises a furin cleavage site. In certain embodiments, the IL21 comprises the amino acid sequence of MERIVICLMVIFLGTLVHKSSSQGQDRHMIRMRQLIDIVDQLKNYVNDLVPEFLPAPED VETNCEWSAFSCFQKAQLKSANTGNNERIINVSIKKLKRKPPSTNAGRRQKHRLTCPSC DSYEKKPPKEFLERFKSLLQKMIHQHLSSRTHGSEDSRRKR (SEQ ID NO: 413). An exemplary nucleic acid sequence encoding SEQ ID NO: 413 is

(SEQ ID NO: 414)
ATGGAACGGATCGTGATCTGCCTGATGGTCATCTTCCTGGGCACCCTGGT
GCACAAGAGCAGCTCTCAGGGCCAAGACCGGCACATGATCCGGATGAGAC
AGCTGATCGACATCGTGGACCAGCTGAAGAACTACGTGAACGACCTGGTG
CCTGAGTTCCTGCCTGCTCCTGAGGACGTGGAAACAAACTGCGAGTGGAG
CGCCTTCAGCTGCTTCCAGAAGGCCCAGCTGAAAAGCGCCAACACCGGCA
ACAACGAGCGGATCATCAACGTGTCCATCAAGAAGCTGAAGCGGAAGCCT
CCTAGCACCAACGCCGGAAGAAGGCAGAAGCACAGACTGACCTGTCCTAG
CTGCGACAGCTACGAGAAGAAGCCTCCAAAAGAGTTCCTGGAACGGTTCA
AGAGCCTGCTGCAGAAGATGATCCACCAGCACCTGAGCAGCAGAACCCAC
GGCAGCGAGGACTCCAGAAGAAAACGC.

In certain embodiments, the IL7 comprises the amino acid sequence of DCDIEGKDGKQYESVLMVSIDQLLDSMKEIGSNCLNNEFNFFKRHICDANKEGMFLFRA ARKLRQFLKMNSTGDFDLHLLKVSEGTTILLNCTGQVKGRKPAALGEAQPTKSLEENK SLKEQKKLNDLCFLKRLLQEIKTCWNKILMGTKEH (SEQ ID NO: 394) An exemplary nucleic acid sequence encoding SEQ ID NO: 394 is GACTGTGATATCGAGGGCAAAGACGGCAAGCAGTACGAGAGCGTGCTGATGGTGTC CATCGACCAGCTGCTGGACAGCATGAAGGAAATCGGCAGCAACTGCCTGAACAACG AGTTCAACTTCTTCAAGCGGCACATCTGCGACGCCAACAAAGAAGGCATGTTCCTG TTCAGAGCCGCCAGAAAGCTGCGGCAGTTCCTGAAGATGAACAGCACCGGCGACTT CGACCTGCATCTGCTGAAAGTGTCTGAGGGCACCACCATCCTGCTGAATTGCACCG GCCAAGTGAAGGGCAGAAAGCCTGCTGCTCTGGGAGAAGCCCAGCCTACCAAGAG CCTGGAAGAGAACAAGTCCCTGAAAGAGCAGAAGAAGCTGAACGACCTCTGCTTCC TGAAGCGGCTGCTGCAAGAGATCAAGACCTGCTGGAACAAGATCCTGATGGGCACC AAAGAGCAC (SEQ ID NO: 393). In certain embodiments, a nucleic acid encoding SEQ ID NO: 394 comprises a sequence that is at least 90%, at least 91%, at least 92%, at least 93%, at least 94%, at least 95%, at least 96%, at least 97%, at least 98%, or at least 99% identical to SEQ ID NO: 393.

In certain embodiments, the IL12p70 comprises the amino acid sequence of MCHQQLVISWFSLVFLASPLVAIWELKKDVYVVELDWYPDAPGEMVVLTCDTPEEDGI TWTLDQSSEVLGSGKTLTIQVKEFGDAGQYTCHKGGEVLSHSLLLLHKKEDGIWSTDIL KDQKEPKNKTFLRCEAKNYSGRFTCWWLTTISTDLTFSVKSSRGSSDPQGVTCGAATLS AERVRGDNKEYEYSVECQEDSACPAAEESLPIEVMVDAVHKLKYENYTSSFFIRDIIKPD PPKNLQLKPLKNSRQVEVSWEYPDTWSTPHSYFSLTFCVQVQGKSKREKKDRVFTDKT SATVICRKNASISVRAQDRYYSSSWSEWASVPCSGGGSGGGSGGGSGGGSRNLPVATP DPGMFPCLHHSQNLLRAVSNMLQKARQTLEFYPCTSEEIDHEDITKDKTSTVEACLPLE LTKNESCLNSRETSFITNGSCLASRKTSFMMALCLSSIYEDLKMYQVEFKTMNAKLLMD PKRQIFLDQNMLAVIDELMQALNFNSETVPQKSSLEEPDFYKTKIKLCILLHAFRIRAVTI DRVMSYLNAS (SEQ ID NO: 293). An exemplary nucleic acid sequence encoding SEQ ID NO: 293 is ATGTGTCACCAGCAGCTGGTCATCAGCTGGTTCAGCCTGGTGTTCCTGGCCTCTCCT CTGGTGGCCATCTGGGAGCTGAAGAAAGACGTGTACGTGGTGGAACTGGACTGGTA TCCCGATGCTCCTGGCGAGATGGTGGTGCTGACCTGCGATACCCCTGAAGAGGACG GCATCACCTGGACACTGGATCAGTCTAGCGAGGTGCTCGGCAGCGGCAAGACCCTG ACCATCCAAGTGAAAGAGTTTGGCGACGCCGGCCAGTACACCTGTCACAAAGGCGG AGAAGTGCTGAGCCACAGCCTGCTGCTGCTCCACAAGAAAGAGGATGGCATTTGGA GCACCGACATCCTGAAGGACCAGAAAGAGCCCAAGAACAAGACCTTCCTGAGATG CGAGGCCAAGAACTACAGCGGCCGGTTCACATGTTGGTGGCTGACCACCATCAGCA CCGACCTGACCTTCAGCGTGAAGTCCAGCAGAGGCAGCAGTGATCCTCAGGGCGTT ACATGTGGCGCCGCTACACTGTCTGCCGAAAGAGTGCGGGGCGACAACAAAGAATA CGAGTACAGCGTGGAATGCCAAGAGGACAGCGCCTGTCCAGCCGCCGAAGAGTCTC TGCCTATCGAAGTGATGGTGGACGCCGTGCACAAGCTGAAGTACGAGAACTACACC TCCAGCTTTTTCATCCGGGACATCATCAAGCCCGATCCTCCAAAGAACCTGCAGCTG AAGCCTCTGAAGAACAGCAGACAGGTGGAAGTGTCCTGGGAGTACCCCGACACCTG GTCTACACCCCACAGCTACTTCAGCCTGACCTTTTGCGTGCAAGTGCAGGGCAAGTC CAAGCGCGAGAAAAAGGACCGGGTGTTCACCGACAAGACCAGCGCCACCGTGATC TGCAGAAAGAACGCCAGCATCAGCGTCAGAGCCCAGGACCGGTACTACAGCAGCTC TTGGAGCGAATGGGCCAGCGTGCCATGTTCTGGCGGAGGAAGCGGTGGCGGATCAG GTGGTGGATCTGGCGGCGGATCTAGAAACCTGCCTGTGGCCACTCCTGATCCTGGC ATGTTCCCTTGTCTGCACCACAGCCAGAACCTGCTGAGAGCCGTGTCCAACATGCTG CAGAAGGCCAGACAGACCCTGGAATTCTACCCCTGCACCAGCGAGGAAATCGACCA CGAGGACATCACCAAGGATAAGACCAGCACCGTGGAAGCCTGCCTGCCTCTGGAAC TGACCAAGAACGAGAGCTGCCTGAACAGCCGGGAAACCAGCTTCATCACCAACGGC TCTTGCCTGGCCAGCAGAAAGACCTCCTTCATGATGGCCCTGTGCCTGAGCAGCATC TACGAGGACCTGAAGATGTACCAGGTGGAATTCAAGACCATGAACGCCAAGCTGCT GATGGACCCCAAGCGGCAGATCTTCCTGGACCAGAATATGCTGGCCGTGATCGACG AGCTGATGCAGGCCCTGAACTTCAACAGCGAGACAGTGCCCCAGAAGTCTAGCCTG GAAGAACCCGACTTCTACAAGACCAAGATCAAGCTGTGCATCCTGCTGCACGCCTT CCGGATCAGAGCCGTGACCATCGACAGAGTGATGAGCTACCTGAACGCCTCT (SEQ ID NO: 294). In certain embodiments, a nucleic acid encoding SEQ ID NO: 293 comprises a sequence that is at least 90%, at least 91%, at least 92%, at least 93%, at least 94%, at least 95%, at least 96%, at least 97%, at least 98%, or at least 99% identical to SEQ ID NO: 294.

In general, a cell (e.g., an immune cell or a stem cell) is engineered to produce two or more cytokines, including at least one of the cytokines being in a membrane-cleavable chimeric protein format (e.g., “S” in the formula S-C-MT or MT-C-S).

In some embodiments, a cell is engineered to produce at least one membrane-cleavable chimeric protein where the secretable effector molecule (e.g., “S” in the formula S-C-MT or MT-C-S) is IL15, IL12, an IL12p70 fusion protein, IL18, or IL21.

In some embodiments, a cell is engineered to produce at least one membrane-cleavable chimeric protein where the secretable effector molecule (e.g., “S” in the formula S-C-MT or MT-C-S) is IL15. In some embodiments, a cell is engineered to produce at least one membrane-cleavable chimeric protein where the secretable effector molecule is IL15 and the cell is further engineered to produce one or more additional cytokines. In some embodiments, a cell is engineered to produce at least one membrane-cleavable chimeric protein where the secretable effector molecule is IL15 and the cell is further engineered to produce IL12, an IL12p70 fusion protein, IL18, or IL21. In some embodiments, a cell is engineered to produce at least one membrane-cleavable chimeric protein where the secretable effector molecule is IL15 and the cell is further engineered to produce IL12. In some embodiments, a cell is engineered to produce at least one membrane-cleavable chimeric protein where the secretable effector molecule is IL15 and the cell is further engineered to produce an IL12p70 fusion protein.

In some embodiments, a cell is engineered to produce at least one membrane-cleavable chimeric protein where the secretable effector molecule (e.g., “S” in the formula S-C-MT or MT-C-S) is IL15 and the cell is further engineered to produce one or more additional membrane-cleavable chimeric proteins. In some embodiments, a cell is engineered to produce at least one membrane-cleavable chimeric protein where the secretable effector molecule (e.g., “S” in the formula S-C-MT or MT-C-S) is IL15 and the cell is further engineered to produce one or more additional membrane-cleavable chimeric proteins including IL12, an IL12p70 fusion protein, IL18, and IL21. In some embodiments, a cell is engineered to produce at least one membrane-cleavable chimeric protein where the secretable effector molecule (e.g., “S” in the formula S-C-MT or MT-C-S) is IL15 and the cell is further engineered to produce an additional membrane-cleavable chimeric proteins including IL12p70. In some embodiments, a cell is engineered to produce at least one membrane-cleavable chimeric protein where the secretable effector molecule (e.g., “S” in the formula S-C-MT or MT-C-S) is IL15 and the cell is further engineered to produce IL21.

In some embodiments, a cell is engineered to produce at least one membrane-cleavable chimeric protein where the secretable effector molecule (e.g., “S” in the formula S-C-MT or MT-C-S) is an IL12p70. In some embodiments, a cell is engineered to produce at least one membrane-cleavable chimeric protein where the secretable effector molecule is IL12p70 and the cell is further engineered to produce one or more additional cytokines. In some embodiments, a cell is engineered to produce at least one membrane-cleavable chimeric protein where the secretable effector molecule is IL12p70 and the cell is further engineered to produce IL15, IL18, or IL21. In some embodiments, a cell is engineered to produce at least one membrane-cleavable chimeric protein where the secretable effector molecule is IL12p70 and the cell is further engineered to produce IL15.

In some embodiments, a cell is engineered to produce at least one membrane-cleavable chimeric protein where the secretable effector molecule (e.g., “S” in the formula S-C-MT or MT-C-S) is IL12p70 and the cell is further engineered to produce one or more additional membrane-cleavable chimeric proteins. In some embodiments, a cell is engineered to produce at least one membrane-cleavable chimeric protein where the secretable effector molecule (e.g., “S” in the formula S-C-MT or MT-C-S) is IL12p70 and the cell is further engineered to produce one or more additional membrane-cleavable chimeric proteins including IL15, IL18, and IL21. In some embodiments, a cell is engineered to produce at least one membrane-cleavable chimeric protein where the secretable effector molecule (e.g., “S” in the formula S-C-MT or MT-C-S) is IL12p70 and the cell is further engineered to produce an additional membrane-cleavable chimeric proteins including IL15.

A cell can also be further engineered to express additional proteins in addition to the cytokines and/or the membrane-cleavable chimeric proteins having the formula S-C-MT or MT-C-S described herein. As provided herein, an immunoresponsive cell may be engineered to express a chimeric antigen receptor (CAR). As provided herein, an immunoresponsive cell may be engineered to express a chimeric antigen receptor (CAR) that binds to GPC3. As provided herein, an immunoresponsive cell may be engineered to express a chimeric antigen receptor (CAR) that binds to a target selected from: CEA, CEACAM1, CEACAM5, and CEACAM6. CEACAM5. In particular embodiments, the CAR binds to CEACAM5. In some embodiments, the chimeric antigen receptor (CAR) (e.g., that that binds to CEACAM5) is an activating CAR (aCAR). In some embodiments, an immunoresponsive cell is engineered to further express a second CAR. The second CAR may be an inactivating CAR (iCAR). The iCAR may be an iCAR that binds to V-set and immunoglobulin domain-containing protein 2 (UniProt Accession No. Q961Q7, “VSIG2”).

Also as provided herein, an immunoresponsive cell may be engineered to express an ACP that includes a synthetic transcription factor.

A CAR can include an antigen-binding domain, such as an antibody, an antigen-binding fragment of an antibody, a F(ab) fragment, a F(ab′) fragment, a single chain variable fragment (scFv), or a single-domain antibody (sdAb). An antigen recognizing receptors can include an scFv. An scFv can include a heavy chain variable domain (VH) and a light chain variable domain (VL), which can be separated by a peptide linker. For example, an scFv can include the structure VH-L-VL or VL-L-VH, wherein VH is the heavy chain variable domain, L is the peptide linker, and VL is the light chain variable domain. In certain embodiments, the peptide linker is a gly-ser linker. In certain embodiments, the peptide linker is a (GGGGS)₃ linker (SEQ ID NO: 223) comprising the sequence of GGGGSGGGGSGGGGS (SEQ ID NO: 223). An exemplary nucleic acid sequence encoding SEQ ID NO: 223 is GGCGGCGGAGGATCTGGCGGAGGTGGAAGTGGCGGAGGCGGATCT (SEQ ID NO: 224) or GGCGGCGGAGGAAGCGGAGGCGGAGGATCCGGTGGTGGTGGATCT (SEQ ID NO: 332). In certain embodiments, a nucleic acid encoding SEQ ID NO: 223 comprises a sequence that is at least 90%, at least 91%, at least 92%, at least 93%, at least 94%, at least 95%, at least 96%, at least 97%, at least 98%, or at least 99% identical to SEQ ID NO: 224 or SEQ ID NO: 332. In some embodiments, a VH and a VL of an aCAR is separated by a peptide linker having the sequence SEQ ID NO: 223. In some embodiments, a VH and a VL of an iCAR is separated by a peptide linker having the sequence GSTSGSGKPGSGEGSTKG (SEQ ID NO: 395). An exemplary nucleic acid sequence encoding GSTSGSGKPGSGEGSTKG (SEQ ID NO: 395) is

(SEQ ID NO: 404)
GGCAGCACAAGCGGCTCTGGAAAACCTGGATCTGGCGAGGGCTCTACCAA
GGGC

A CAR can have one or more intracellular signaling domains. In some embodiments, an activating CAR (aCAR) can activate immune cells and contain an intracellular signaling domain, such as a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceRlg intracellular signaling domain, a NKG2D intracellular signaling domain, an EAT-2 intracellular signaling domain, fragments thereof, combinations thereof, or combinations of fragments thereof. In some embodiments, the aCAR comprises a CD28 intracellular signaling domain. In some embodiments, the aCAR comprises a CD3zeta intracellular signaling domain. In some embodiments, the aCAR comprises both a CD28 ICD and a CD3zeta CD. In particular embodiments, the CD28 CD comprises SEQ ID NO: 267 and the CD3zeta ICD comprises SEQ ID NO: 277. In some embodiments, an inhibitory CAR (iCAR) can inhibit immune cells and contain a SIRPα or LIR1 intracellular signaling domain. In particular embodiments, the iCAR contains a SIRPα ICD, optionally having the sequence SEQ ID NO: 385. In some embodiments, the intracellular signaling domain comprises a sequence from Table 6A.

TABLE 6A

| Amino Acid Sequence | Nucleotide Sequence | Description |
|---|---|---|
| KSRQTPPLASVEMEAMEALP | AAGTCCAGACAGACACCTCCTCTGGCCAGCGTGG | IL 15Rα ICD |
| VTWGTSSRDEDLENCSHHL | AAATGGAAGCCATGGAAGCTCTGCCTGTGACCTG | (activating) |
| (SEQ ID NO: 265) | GGGCACCAGCTCCAGAGATGAGGACCTGGAAAAC | |
| | TGCTCCCACCACCTG | |
| | (SEQ ID NO: 266) | |
| RSKRSRLLHSDYMNMTPRR | CGGAGCAAGAGAAGCAGACTGCTGCACAGCGACT | CD28 ICD |
| PGPTRKHYQPYAPPRDFAA | ACATGAACATGACCCCTAGACGGCCCGGACCTAC | (activating) |
| YRS | CAGAAAGCACTACCAGCCTTACGCTCCTCCTAGAG | |
| (SEQ ID NO: 267) | ACTTCGCCGCCTACCGGTCC (SEQ ID NO: 268) | |
| AL YLLRRDQRLPPDAHKPPG | GCTCTGTATCTGCTGCGGAGGGACCAAAGACTGCC | OX40 ICD |
| GGSFRTPIQEEQADAHSTLA | TCCTGATGCTCACAAGCCTCCAGGCGGAGGCAGCT | (activating) |
| KI | TCAGAACCCCTATCCAAGAGGAACAGGCCGACGC | |
| (SEQ ID NO: 269) | TCACAGCACCCTGGCCAAGATT (SEQ ID NO: 270) | |
| KRGRKKLLYIFKQPFMRPVQ | AAGCGGGGCAGAAAGAAGCTGCTGTACATCTTCA | 4-1BB ICD |
| TTQEEDGCSCRFPEEEEGGC | AGCAGCCCTTCATGCGGCCCGTGCAGACCACACA | (activating) |
| EL | AGAGGAAGATGGCTGCTCCTGCAGATTCCCCGAG | |
| (SEQ ID NO: 271) | GAAGAAGAAGGCGGCTGCGAGCTG | |
| | (SEQ ID NO: 272) | |
| | OR | |
| | AAGCGGGGCAGAAAGAAGCTGCTGTACATCTTCA | |
| | AGCAGCCCTTCATGCGGCCCGTGCAGACCACACA | |
| | AGAGGAAGATGGCTGCAGCTGTCGGTTCCCCGAG | |
| | GAAGAAGAAGGCGGCTGCGAGCTG (SEQ ID NO: | |
| | 339) | |
| RKRTRERASRASTWEGRRR | CGGAAGCGGACAAGAGAGAGAGCCAGCAGAGCCT | Nkp46 ICD |
| LNTQTL | CTACCTGGGAGGGAAGAAGAAGGCTGAACACCCA | (activating) |
| (SEQ ID NO: 273) | GACACTC | |
| | (SEQ ID NO: 274) | |
| WRRKRKEKQSETSPKEFLTI | TGGCGCCGCAAGCGGAAAGAGAAGCAGTCTGAGA | 2B4 ICD |
| YEDVKDLKTRRNHEQEQTF | CAAGCCCCAAAGAGTTCCTGACCATCTACGAGGA | (activating) |
| PGGGSTIYSMIQSQSSAPTSQ | CGTGAAGGACCTGAAAACCCGGCGGAACCACGAG | |
| EPAYTLYSLIQPSRKSGSRKR | CAAGAGCAGACCTTTCCTGGCGGCGGAAGCACCA | |
| NHSPSFNSTIYEVIGKSQPKA | TCTACAGCATGATCCAGAGCCAGTCTAGCGCCCCT | |
| QNPARLSRKELENFDVYS | ACCAGCCAAGAGCCTGCCTACACACTGTACTCCCT | |
| (SEQ ID NO: 275) | GATCCAGCCTAGCAGAAAGAGCGGCAGCCGGAAG | |
| | AGAAATCACAGCCCCAGCTTCAACAGCACGATCT | |
| | ACGAAGTGATCGGCAAGAGCCAGCCAAAGGCTCA | |
| | GAACCCTGCCAGGCTGAGCCGGAAAGAGCTGGAA | |
| | AACTTCGACGTGTACAGC | |
| | (SEQ ID NO: 276) | |
| RVKFSRSADAPAYKQGQNQ | AGAGTGAAGTTCAGCAGAAGCGCCGACGCACCCG | CD3z mut |
| LYNELNLGRREEYDVLDKR | CCTATAAGCAGGGACAGAACCAGCTGTACAACGA | (activating) |
| RGRDPEMGGKPRRKNPQEG | GCTGAACCTGGGGAGAAGAGAAGAGTACGACGTG | |
| LYNELQKDKMAEAYSEIGM | CTGGACAAGCGGAGAGGCAGAGATCCTGAGATGG | |
| KGERRRGKGHDGLYQGLST | GCGGCAAGCCCAGACGGAAGAATCCTCAAGAGGG | |
| ATKDTYDALHMQALPPR | CCTGTATAATGAGCTGCAGAAAGACAAGATGGCC | |
| (SEQ ID NO: 277) | GAGGCCTACAGCGAGATCGGAATGAAGGGCGAGC | |
| | GCAGAAGAGGCAAGGGACACGATGGACTGTACCA | |
| | GGGCCTGAGCACCGCCACCAAGGATACCTATGAT | |
| | GCCCTGCACATGCAGGCCCTGCCTCCAAGA (SEQ | |
| | ID NO: 278) | |
| | OR | |
| | AGAGTGAAGTTCAGCAGGAGCGCAGACGCCCCCG | |
| | CGTACAAGCAGGGCCAGAACCAGCTCTATAACGA | |
| | GCTCAATCTAGGACGAAGAGAGGAGTACGATGTT | |
| | TTGGACAAGAGACGTGGCCGGGACCCTGAGATGG | |
| | GGGGAAAGCCGAGAAGGAAGAACCCTCAGGAAG | |
| | GCCTGTACAATGAACTGCAGAAAGATAAGATGGC | |
| | GGAGGCCTACAGTGAGATTGGGATGAAAGGCGAG | |
| | CGCCGGAGGGGCAAGGGGCACGATGGCCTTTACC | |
| | AGGGTCTCAGTACAGCCACCAAGGACACCTACGA | |
| | CGCCCTTCACATGCAGGCCCTGCCCCCTCGC (SEQ | |
| | ID NO: 334) | |
| RVKFSRSADAPAYQQGQNQ | AGAGTGAAGTTCAGCAGATCCGCCGATGCTCCCGC | CD3z |
| LYNELNLGRREEYDVLDKR | CTATCAGCAGGGACAGAACCAGCTGTACAACGAG | (activating) |
| RGRDPEMGGKPRRKNPQEG | CTGAACCTGGGGAGAAGAGAAGAGTACGACGTGC | |
| LYNELQKDKMAEAYSEIGM | TGGACAAGCGGAGAGGCAGAGATCCTGAGATGGG | |
| KGERRRGKGHDGLYQGLST | CGGCAAGCCCAGACGGAAGAATCCTCAAGAGGGC | |
| ATKDTYDALHMQALPPR | CTGTATAATGAGCTGCAGAAAGACAAGATGGCCG | |
| (SEQ ID NO: 279) | AGGCCTACAGCGAGATCGGAATGAAGGGCGAGCG | |
| | CAGAAGAGGCAAGGGACACGATGGACTGTACCAG | |
| | GGCCTGAGCACCGCCACCAAGGATACCTATGATG | |
| | CCCTGCACATGCAGGCCCTGCCTCCAAGA (SEQ ID | |
| | NO: 280) | |
| VRIRQKKAQGSTSSTRLHEP | GTGAGAATCAGACAGAAGAAGGCCCAGGGCAGCA | SIRPa |
| EKNAREITQDTNDITYADLN | CCAGCAGCACCAGACTGCACGAGCCCGAGAAGAA | (inhibitory) |
| LPKGKKPAPQAAEPNNHTE | CGCCAGAGAGATCACCCAGGACACCAACGACATC | |
| YASIQTSPQPASEDTLTYAD | ACCTACGCCGACCTGAACCTGCCCAAGGGCAAGA | |
| LDMVHLNRTPKQPAPKPEPS | AGCCCGCCCCCCAGGCCGCCGAGCCCAACAACCA | |
| FSEYASVQVPRK (SEQ ID | CACCGAGTACGCCAGCATCCAGACCAGCCCCCAG | |
| NO: 385) | CCCGCCAGCGAGGACACCCTGACCTACGCCGACCT | |
| | GGACATGGTGCACCTGAACAGAACCCCCAAGCAG | |
| | CCCGCCCCCAAGCCCGAGCCCAGCTTCAGCGAGTA | |
| | CGCCAGCGTGCAGGTGCCCAGAAAG (SEQ ID NO: | |
| | 384) | |
| LRHRRQGKHWTSTQRKADF | TTGCGCCACAGACGGCAGGGAAAGCACTGGACTA | LIR1 |
| QHPAGAVGPEPTDRGLQWR | GTACGCAGAGGAAAGCGGACTTCCAGCATCCCGC | (inhibitory) |
| SSPAADAQEENLYAAVKHT | AGGAGCCGTGGGGCCTGAACCCACTGATCGCGGC | |
| QPEDGVEMDTRSPHDEDPQ | CTTCAATGGAGGTCTAGCCCGGCGGCAGACGCAC | |
| AVTYAEVKHSRPRREMASP | AAGAGGAAAACTTGTACGCAGCCGTTAAGCACAC | |
| PSPLSGEFLDTKDRQAEEDR | CCAACCGGAGGACGGCGTTGAGATGGATACCCGC | |
| QMDTEAAASEAPQDVTYAQ | TCCCCTCACGATGAAGACCCTCAAGCAGTCACTTA | |
| LHSLTLRREATEPPPSQEGPS | CGCGGAAGTAAAGCATAGCCGCCCCAGACGGGAA | |
| PAVPSIYATLAIH | ATGGCTAGCCCGCCGTCCCCCCTTAGCGGGGAATT | |
| (SEQ ID NO: 387) | TCTGGACACTAAAGATAGGCAGGCGGAAGAGGAC | |
| | CGCCAAATGGATACAGAGGCGGCGGCAAGTGAAG | |
| | CACCTCAAGACGTTACTTACGCTCAACTTCACAGC | |
| | CTTACCCTCAGGCGAGAAGCGACTGAACCACCCCC | |
| | TTCCCAAGAAGGGCCAAGCCCAGCGGTTCCTTCTA | |
| | TCTATGCTACTCTTGCTATTCAC | |
| | (SEQ ID NO: 388) | |

In some embodiments, a CAR can also comprise a spacer region that links the extracellular antigen-binding domain to the transmembrane domain. The spacer region may be flexible enough to allow the antigen-binding domain to orient in different directions to facilitate antigen recognition. In some embodiments, the spacer region may be a hinge from a human protein. For example, the hinge may be a human Ig (immunoglobulin) hinge, including without limitation an IgG4 hinge, an IgG2 hinge, a CD8a hinge, or an IgD hinge. In some embodiments, the spacer region may comprise an IgG4 hinge, an IgG2 hinge, an IgD hinge, a CD28 hinge, a KIR2DS2 hinge, an LNGFR hinge, or a PDGFR-beta extracellular linker. In some embodiments, the spacer region comprises a sequence from Table 6B.

TABLE 6B
Exemplary Hinges and Spacer Sequences

| Amino Acid Sequence | Nucleic Acid Sequence | Description |
|---|---|---|
| TTTPAPRPPTPAPTIALQPLSLRPE | ACAACAACCCCTGCTCCTAGACCT | CD8 hinge (S2L) |
| ACRPAAGGAVHTRGLDFACD | CCTACACCAGCTCCTACAATCGCC | |
| (SEQ ID NO: 226) | CTGCAGCCTCTGTCTCTGAGGCCA | |
| | GAAGCTTGTAGACCAGCTGCTGG | |
| | CGGAGCCGTGCATACAAGAGGAC | |
| | TGGACTTCGCCTGTGAT (SEQ ID | |
| | NO: 227) | |
| GALSNSIMYFSHFVPVFLPAKPTT | GGCGCCCTGAGCAACAGCATCAT | CD8 hinge (FA) |
| TPAPRPPTPAPTIASQPLSLRPEAC | GTACTTCAGCCACTTCGTGCCCGT | |
| RPAAGGAVHTRGLDFACD (SEQ | GTTTCTGCCCGCCAAGCCTACAAC | |
| ID NO: 228) | AACCCCTGCTCCTAGACCTCCTAC | |
| OR | ACCAGCTCCTACAATCGCCAGCCA | |
| ALSNSIMYFSHFVPVFLPAKPTTT | GCCTCTGTCTCTGAGGCCAGAAGC | |
| PAPRPPTPAPTIASQPLSLRPEACR | TTGTAGACCTGCTGCAGGCGGAG | |
| PAAGGAVHTRGLDFACD (SEQ ID | CCGTGCATACAAGAGGACTGGAT | |
| NO: 353) | TTCGCCTGCGAC (SEQ ID NO: 229) | |
| | OR | |
| | GCCCTGAGCAACAGCATCATGTA | |
| | CTTCAGCCACTTCGTGCCCGTGTT | |
| | TCTGCCCGCCAAGCCTACAACAAC | |
| | CCCTGCTCCTAGACCTCCTACACC | |
| | AGCTCCTACAATCGCCAGCCAGCC | |
| | TCTGTCTCTGAGGCCAGAAGCTTG | |
| | TAGACCTGCTGCAGGCGGAGCCG | |
| | TGCATACAAGAGGACTGGATTTC | |
| | GCCTGCGAC (SEQ ID NO: 335) | |
| AAAIEVMYPPPYLDNEKSNGTIIH | GCAGCAGCTATCGAGGTGATGTA | CD28 hinge |
| VKGKHLCPSPLFPGPSKP (SEQ ID | TCCTCCGCCCTACCTGGATAATGA | |
| NO: 246) | AAAGAGTAATGGGACTATCATTC | |
| | ATGTAAAAGGGAAGCATCTTTGTC | |
| | CTTCTCCCCTTTTCCCCGGTCCGTC | |
| | TAAACCT (SEQ ID NO: 247) | |
| ESKYGPPCPSCP (SEQ ID NO: 248) | GAAAGCAAGTACGGTCCACCTTG | IgG4 minimal hinge |
| | CCCTAGCTGTCCG (SEQ ID NO: | |
| | 249) | |
| ESKYGPPAPSAP (SEQ ID NO: 250) | GAATCCAAGTACGGCCCCCCAGC | IgG4 minimal hinge, |
| | GCCTAGTGCCCCA (SEQ ID NO: | no disulfides |
| | 251) | |
| ESKYGPPCPPCP (SEQ ID NO: 252) | GAATCTAAATATGGCCCGCCATGC | IgG4 S228P minimal |
| | CCGCCTTGCCCA (SEQ ID NO: 253) | hinge, enhanced |
| | | disulfide |
| | | formation |
| EPKSCDKTHTCP (SEQ ID NO: | GAACCGAAGTCTTGTGATAAAAC | IgG1 minimal |
| 254) | TCATACGTGCCCG (SEQ ID NO: | hinge |
| | 255) | |
| AAAFVPVFLPAKPTTTPAPRPPTP | GCTGCTGCTTTCGTACCCGTGTTC | Extended CD8a |
| APTIASQPLSLRPEACRPAAGGAV | CTCCCTGCTAAGCCTACGACTACC | hinge |
| HTRGLDFACDIYIWAPLAGTCGV | CCCGCACCGAGACCACCCACGCC | |
| LLLSLVITLYCNHRN (SEQ ID NO: | AGCACCCACGATTGCTAGCCAGC | |
| 256) | CCCTTAGTTTGCGACCAGAAGCTT | |
| | GTCGGCCTGCTGCTGGTGGCGCGG | |
| | TACATACCCGCGGCCTTGATTTTG | |
| | CTTGCGATATATATATCTGGGCGC | |
| | CTCTGGCCGGAACATGCGGGGTC | |
| | CTCCTCCTTTCTCTGGTTATTACTC | |
| | TCTACTGTAATCACAGGAAT (SEQ | |
| | ID NO: 257) | |
| ACPTGLYTHSGECCKACNLGEGV | GCCTGCCCGACCGGGCTCTACACT | LNGFR hinge |
| AQPCGANQTVCEPCLDSVTFSDV | CATAGCGGGGAATGTTGTAAGGC | |
| VSATEPCKPCTECVGLQSMSAPC | ATGTAACTTGGGTGAGGGCGTCG | |
| VEADDAVCRCAYGYYQDETTGR | CACAGCCCTGCGGAGCTAACCAA | |
| CEACRVCEAGSGLVFSCQDKQNT | ACAGTGTGCGAACCCTGCCTCGAT | |
| VCEECPDGTYSDEADAEC (SEQ | AGTGTGACGTTCTCTGATGTTGTA | |
| ID NO: 258) | TCAGCTACAGAGCCTTGCAAACC | |
| | ATGTACTGAGTGCGTTGGACTTCA | |
| | GTCAATGAGCGCTCCATGTGTGGA | |
| | GGCAGATGATGCGGTCTGTCGAT | |
| | GTGCTTACGGATACTACCAAGAC | |
| | GAGACAACAGGGCGGTGCGAGGC | |
| | CTGTAGAGTTTGTGAGGCGGGCTC | |
| | CGGGCTGGTGTTTTCATGTCAAGA | |
| | CAAGCAAAATACGGTCTGTGAAG | |
| | AGTGCCCTGATGGCACCTACTCAG | |
| | ACGAAGCAGATGCAGAATGC | |
| | (SEQ ID NO: 259) | |
| ACPTGLYTHSGECCKACNLGEGV | GCCTGCCCTACAGGACTCTACACG | Truncated LNGFR |
| AQPCGANQTVC (SEQ ID NO: 260) | CATAGCGGTGAGTGTTGTAAAGC | hinge (TNFR-Cys1) |
| | ATGCAACCTCGGGGAAGGTGTAG | |
| | CCCAGCCATGCGGGGCTAACCAA | |
| | ACCGTTTGC (SEQ ID NO: 261) | |
| AVGQDTQEVIVVPHSLPFKV | GCTGTGGGCCAGGACACGCAGGA | PDGFR-beta |
| (SEQ ID NO: 262) | GGTCATCGTGGTGCCACACTCCTT | extracellular |
| | GCCCTTTAAGGTG (SEQ ID NO: | linker |
| | 263) | |
| YPPVIVEMNSSVEAIEGSHVSLLC | TACCCTCCAGTGATCGTGGAAATG | MAG hinge |
| GADSNPPPLLTWMRDGTVLREA | AACAGCAGCGTGGAAGCCATCGA | |
| VAESLLLELEEVTPAEDGVYACL | GGGCTCTCATGTGTCTCTGCTGTG | |
| AENAYGQDNRTVGLSVMYAPW | TGGCGCCGACAGCAATCCTCCTCC | |
| KPTVNGTMVAVEGETVSILCSTQ | TCTGCTGACCTGGATGAGAGATG | |
| SNPDPILTIFKEKQILSTVIYESELQ | GCACCGTGCTGAGAGAAGCCGTG | |
| LELPAVSPEDDGEYWCVAENQY | GCCGAATCTCTGCTGCTGGAACTG | |
| GQRATAFNLSVEFAPVLLLESHC | GAAGAAGTGACCCCTGCCGAGGA | |
| AAARDTVQCLCVVKSNPEPSVAF | TGGCGTGTACGCTTGTCTGGCCGA | |
| ELPSRNVTVNESEREFVYSERSGL | GAATGCCTACGGCCAGGACAATA | |
| VLTSILTLRGQAQAPPRVICTARN | GAACCGTGGGCCTGTCCGTGATGT | |
| LYGAKSLELPFQGAHRLMWAKI | ACGCCCCTTGGAAGCCTACCGTGA | |
| GP (SEQ ID NO: 264) | ACGGCACAATGGTGGCCGTGGAA | |
| | GGCGAGACAGTGTCCATCCTGTGT | |
| | AGCACCCAGAGCAACCCCGATCC | |
| | TATCCTGACCATCTTCAAAGAGAA | |
| | GCAGATCCTGAGCACCGTGATCTA | |
| | CGAGAGCGAACTGCAGCTCGAAC | |
| | TGCCCGCTGTGTCCCCAGAGGATG | |
| | ATGGCGAATATTGGTGCGTGGCA | |
| | GAGAACCAGTACGGCCAGAGAGC | |
| | CACCGCCTTCAACCTGAGCGTGGA | |
| | ATTTGCTCCCGTGCTGCTGCTCGA | |
| | GAGCCATTGTGCTGCCGCCAGAG | |
| | ATACCGTGCAGTGCCTGTGTGTGG | |
| | TCAAGTCTAACCCCGAGCCTAGCG | |
| | TGGCCTTTGAGCTGCCCAGCAGAA | |
| | ACGTGACCGTGAATGAGAGCGAG | |
| | CGCGAGTTCGTGTACAGCGAGAG | |
| | ATCTGGACTGGTGCTGACCAGCAT | |
| | CCTGACACTGAGAGGACAGGCTC | |
| | AGGCCCCTCCTAGAGTGATCTGCA | |
| | CCGCCAGAAATCTGTACGGCGCC | |
| | AAGAGCCTGGAACTGCCATTTCA | |
| | GGGCGCCCACAGACTCATGTGGG | |
| | CCAAGATTGGACCT (SEQ ID NO: | |
| | 351) | |

A CAR can have a transmembrane domain, such as a CD8 transmembrane domain, a CD28 transmembrane domain a CD3zeta-chain transmembrane domain, a CD4 transmembrane domain, a 4-130 transmembrane domain, an OX40 transmembrane domain, an ICOS transmembrane domain, a CTLA-4 transmembrane domain, a PD-1 transmembrane domain, a LAG-3 transmembrane domain, a 2B34 transmembrane domain, a BTLA transmembrane domain, an OX40 transmembrane domain, a DAP 10 transmembrane domain, a DAP 12 transmembrane domain, a CDT6a transmembrane domain, a DNAM-1 transmembrane domain, a KIR2DS1 transmembrane domain, a KIR3DS1 transmembrane domain, an NKp44 transmembrane domain, an NKp46 transmembrane domain, an FceRlg transmembrane domain, an NKG2D transmembrane domain, a SIRPα transmembrane domain, a fragments thereof, combinations thereof, or combinations of fragments thereof. A CAR can have a spacer region between the antigen-binding domain and the transmembrane domain. Exemplary transmembrane domain sequences are provided in Table 6C. In particular embodiments, the CAR comprises a SIRPα transmembrane domain, optionally wherein the SIRPα transmembrane domain comprises SEQ ID NO: 383. In particular embodiments, the aCAR comprises a CD28 transmembrane domain.

TABLE 6C

| Amino Acid Sequence | Nucleotide sequence | Description |
|---|---|---|
| VAISTSTVLLCGLSAVSLLACYL | GTGGCCATCAGCACAAGCACCG | IL 15Rα |
| (SEQ ID NO: 230) | TGCTGCTGTGTGGACTGTCTGCC | transmembrane |
| | GTTTCTCTGCTGGCCTGCTACCT | domain |
| | G | |
| | (SEQ ID NO: 231) | |
| FWVLVVVGGVLACYSLLVTVAFI | TTCTGGGTGCTCGTGGTTGTTGG | CD28 |
| IFWV | CGGAGTGCTGGCCTGTTACTCTC | transmembrane |
| (SEQ ID NO: 232) | TGCTGGTCACCGTGGCCTTCATC | domain |
| | ATCTTTTGGGTC | |
| | (SEQ ID NO: 233) | |
| VAAILGLGLVLGLLGPLAIL | GTGGCCGCCATTCTCGGACTGG | OX40 |
| (SEQ ID NO: 234) | GACTTGTTCTGGGACTGCTGGG | transmembrane |
| | ACCTCTGGCCATTCTGCT (SEQ | domain* |
| | ID NO: 235) | |
| VAAILGLGLVLGLLGPLAILL | GTGGCCGCCATTCTCGGACTGG | OX40 |
| (SEQ ID NO: 244) | GACTTGTTCTGGGACTGCTGGG | transmembrane |
| | ACCTCTGGCCATTCTGCTG (SEQ | domain |
| | ID NO: 245) | |
| IYIWAPLAGTCGVLLLSLVIT | ATCTACATCTGGGCCCCTCTGGC | CD8 |
| (SEQ ID NO: 236) | TGGAACATGCGGAGTGTTGCTG | transmembrane |
| | CTGAGCCTGGTCATCACC | domain |
| | (SEQ ID NO: 237) | |
| | OR | |
| | ATCTACATCTGGGCCCCTCTGGC | |
| | TGGAACATGTGGTGTCTTGCTGC | |
| | TGAGCCTGGTCATCACC (SEQ ID | |
| | NO: 338) | |
| IYIWAPLAGTCGVLLLSLVITLY | ATCTACATCTGGGCCCCTCTGGC | CD8 FA |
| CNHR (SEQ ID NO: 242) | TGGAACATGTGGTGTCCTGCTG | transmembrane |
| | CTGAGCCTGGTCATCACCCTGT | domain |
| | ACTGCAACCACCGG (SEQ ID NO: | |
| | 243) | |
| MGLAFLVLVALVWFLVEDWLS | ATGGGCCTCGCCTTTCTGGTGCT | NKp46 |
| (SEQ ID NO: 238) | GGTGGCCCTTGTGTGGTTCCTGG | transmembrane |
| | TGGAAGATTGGCTGAGC | domain |
| | (SEQ ID NO: 239) | |
| FLVIIVILSALFLGTLACFCV | TTCCTGGTCATCATCGTGATCCT | 2B4 |
| (SEQ ID NO: 240) | GAGCGCCCTGTTCCTGGGCACC | transmembrane |
| | CTGGCCTGTTTTTGCGTG | domain |
| | (SEQ ID NO: 241) | |
| IVVGVVCTLLVALLMAALYL | ATCGTGGTGGGCGTGGTGTGCA | SIRPα |
| (SEQ ID NO: 383) | CCCTGCTGGTGGCCCTGCTGAT | transmembrane |
| | GGCCGCCCTGTACCTG (SEQ ID | domain |
| | NO: 382) | |
| VIGILVAVILLLLLLLLLFLI | GTTATAGGGATCCTGGTGGCTG | LIR1 |
| (SEQ ID NO: 389) | TCATACTCCTCTTGCTCCTCTTG | transmembrnae |
| | TTGCTGCTTTTTTTGATA | domain |
| | (SEQ ID NO: 390) | |

In some embodiments, the aCAR antigen-binding domain binds to GPC3. In some embodiments, the aCAR antigen-binding domain that binds to GPC3 includes a heavy chain variable (VH) region and a light chain variable (VL) region, wherein the VH includes: a heavy chain complementarity determining region 1 (CDR-H1) having the amino acid sequence of KNAMN (SEQ ID NO: 199), a heavy chain complementarity determining region 2 (CDR-H2) having the amino acid sequence of RIRNKTNNYATYYADSVKA (SEQ ID NO: 200), and a heavy chain complementarity determining region 3 (CDR-H3) having the amino acid sequence of GNSFAY (SEQ ID NO: 201), and wherein the VL includes: a light chain complementarity determining region 1 (CDR-L1) having the amino acid sequence of KSSQSLLYSSNQKNYLA (SEQ ID NO: 202), a light chain complementarity determining region 2 (CDR-L2) having the amino acid sequence of WASSRES (SEQ ID NO: 203), and a light chain complementarity determining region 3 (CDR-L3) having the amino acid sequence of QQYYNYPLT (SEQ ID NO: 204). In some embodiments, the antigen-binding domain that binds to GPC3 includes a heavy chain complementarity determining region 1 (CDR-H1) having the amino acid sequence of KNAMN (SEQ ID NO: 199). In some embodiments, the antigen-binding domain that binds to GPC3 includes a heavy chain complementarity determining region 2 (CDR-H2) having the amino acid sequence of RIRNKTNNYATYYADSVKA (SEQ ID NO: 200). In some embodiments, the antigen-binding domain that binds to GPC3 includes a heavy chain complementarity determining region 3 (CDR-H3) having the amino acid sequence of GNSFAY (SEQ ID NO: 201). In some embodiments, the antigen-binding domain that binds to GPC3 includes a light chain complementarity determining region 1 (CDR-L1) having the amino acid sequence of KSSQSLLYSSNQKNYLA (SEQ ID NO: 202). In some embodiments, the antigen-binding domain that binds to GPC3 includes a light chain complementarity determining region 2 (CDR-L2) having the amino acid sequence of WASSRES (SEQ ID NO: 203). In some embodiments, the antigen-binding domain that binds to GPC3 includes a light chain complementarity determining region 3 (CDR-L3) having the amino acid sequence of QQYYNYPLT (SEQ ID NO: 204).

In some embodiments, the antigen-binding domain that binds to GPC3 includes a VH region having an amino acid sequence with at least 90%, at least 91%, at least 92%, at least 93%, at least 94%, at least 95%, at least 96%, at least 97%, at least 98%, at least 99%, or 100% identity to the amino acid sequence of EVQLVETGGGMVQPEGSLKLSCAASGFTFNKNAMNWVRQAPGKGLEWVARIRNKTN NYATYYADSVKARFTISRDDSQSMLYLQMNNLKIEDTAMYYCVAGNSFA YWGQGTLVTVSA (SEQ ID NO: 205) or EVQLVESGGGLVQPGGSLRLSCAASGFTFNKNAMNWVRQAPGKGLEWVGRIRNKTNN YATYYADSVKARFTISRDDSKNSLYLQMNSLKTEDTAVYYCVAGNSFAYWGQGTLVT VSA (SEQ ID NO: 206). An exemplary nucleic acid sequence encoding SEQ ID NO: 206 is GAAGTGCAGCTGGTGGAATCTGGCGGAGGACTGGTTCAACCTGGCGGCTCTCTGAG ACTGTCTTGTGCCGCCAGCGGCTTCACCTTCAACAAGAACGCCATGAACTGGGTCCG ACAGGCCCCTGGCAAAGGCCTTGAATGGGTCGGACGGATCCGGAACAAGACCAAC AACTACGCCACCTACTACGCCGACAGCGTGAAGGCCAGGTTCACCATCTCCAGAGA TGACAGCAAGAACAGCCTGTACCTGCAGATGAACTCCCTGAAAACCGAGGACACCG CCGTGTACTATTGCGTGGCCGGCAATAGCTTTGCCTACTGGGGACAGGGCACCCTG GTTACAGTTTCTGCT (SEQ ID NO: 222) or GAAGTGCAGCTGGTTGAATCAGGTGGCGGCCTGGTTCAACCTGGCGGATCTCTGAG ACTGAGCTGTGCCGCCAGCGGCTTCACCTTCAACAAGAACGCCATGAACTGGGTCC GACAGGCCCCTGGCAAAGGCCTTGAATGGGTCGGACGGATCCGGAACAAGACCAA CAACTACGCCACCTACTACGCCGACAGCGTGAAGGCCAGATTCACCATCAGCCGGG ACGACAGCAAGAACAGCCTGTACCTGCAGATGAACTCCCTGAAAACCGAGGACACC GCCGTGTATTATTGCGTGGCCGGCAACAGCTTTGCCTACTGGGGACAGGGAACCCT GGTCACCGTGTCTGCC (SEQ ID NO: 330). In certain embodiments, a nucleic acid encoding SEQ ID NO: 206 comprises a sequence that is at least 90%, at least 91%, at least 92%, at least 93%, at least 94%, at least 95%, at least 96%, at least 97%, at least 98%, or at least 99% identical to SEQ ID NO: 222 or SEQ ID NO: 330.

In some embodiments, the antigen-binding domain that binds to GPC3 includes a VL region having an amino acid sequence with at least 90%, at least 91%, at least 92%, at least 93%, at least 94%, at least 95%, at least 96%, at least 97%, at least 98%, at least 99%, or 100% identity to the amino acid sequence of DIVMSQSPSSLVVSIGEKVTMTCKSSQSLLYSSNQKNYLAWYQQKPGQSPKLLIYWASS RESGVPDRFTGSGSGTDFTLTISSVKAEDLAVYYCQQYYNYPLTFGAGTKLELK (SEQ ID NO: 207), or DIVMTQSPDSLAVSLGERATINCKSSQSLLYSSNQKNYLAWYQQKPGQPPKLLIYWASS RESGVPDRFSGSGSGTDFTLTISSLQAEDVAVYYCQQYYNYPLTFGQGTKLEIK (SEQ ID NO: 208). An exemplary nucleic acid sequence encoding SEQ ID NO: 208 is GACATCGTGATGACACAGAGCCCCGATAGCCTGGCCGTGTCTCTGGGAGAAAGAGC CACCATCAACTGCAAGAGCAGCCAGAGCCTGCTGTACTCCAGCAACCAGAAGAACT ACCTGGCCTGGTATCAGCAAAAGCCCGGCCAGCCTCCTAAGCTGCTGATCTATTGG GCCAGCTCCAGAGAAAGCGGCGTGCCCGATAGATTTTCTGGCTCTGGCAGCGGCAC CGACTTCACCCTGACAATTTCTAGCCTGCAAGCCGAGGACGTGGCCGTGTACTACTG CCAGCAGTACTACAACTACCCTCTGACCTTCGGCCAGGGCACCAAGCTGGAAATCA AA (SEQ ID NO: 221) or GACATCGTGATGACACAGAGCCCCGATAGCCTGGCCGTGTCTCTGGGAGAAAGAGC CACCATCAACTGCAAGAGCAGCCAGAGCCTGCTGTACTCCAGCAACCAGAAGAACT ACCTGGCCTGGTATCAGCAAAAGCCCGGCCAGCCTCCTAAGCTGCTGATCTATTGG GCCAGCTCCAGAGAAAGCGGCGTGCCCGATAGATTTTCTGGCTCTGGCAGCGGCAC CGACTTCACCCTGACAATTTCTAGCCTGCAAGCCGAGGACGTGGCCGTGTATTACTG CCAGCAGTACTACAACTACCCTCTGACCTTCGGCCAGGGCACCAAGCTGGAAATCA AA (SEQ ID NO: 333) or GACATCGTGATGACACAGAGCCCCGATAGCCTGGCCGTGTCTCTGGGAGAAAGAGC CACCATCAACTGCAAGAGCAGCCAGAGCCTGCTGTACTCCAGCAACCAGAAGAACT ACCTGGCCTGGTATCAGCAAAAGCCCGGCCAGCCTCCTAAGCTGCTGATCTATTGG GCCAGCTCCAGAGAAAGCGGCGTGCCCGATAGATTTTCTGGCTCTGGCAGCGGCAC CGACTTCACCCTGACAATTTCTAGCCTGCAAGCCGAGGACGTGGCCGTGTATTACTG CCAGCAGTACTACAACTACCCTCTGACCTTCGGCCAGGGCACCAAGCTGGAAATCA AG (SEQ ID NO: 336). In certain embodiments, a nucleic acid encoding SEQ ID NO: 208 comprises a sequence that is at least 90%, at least 91%, at least 92%, at least 93%, at least 94%, at least 95%, at least 96%, at least 97%, at least 98%, or at least 99% identical to SEQ ID NO: 221 or SEQ ID NO: 336.

In some embodiments, the aCAR antigen-binding domain binds to a target selected from CEA, CEACAM1, CEACAM5, and CEACAM6. In some embodiments, the aCAR antigen-binding domain binds to CEACAM5. In some embodiments, the antigen-binding domain that binds to CEACAM5 includes an scFv having an amino acid sequence with at least 90%, at least 91%, at least 92%, at least 93%, at least 94%, at least 95%, at least 96%, at least 97%, at least 98%, at least 99%, or 100% identity to the amino acid sequence of DIQLTQSPSSLSASVGDRVTITCKASQDVGTSVAWYQQKPGKAPKLLIYWTSTRHTGVP SRFSGSGSGTDFTFTISSLQPEDIATYYCQQYSLYRSFGQGTKVEIKGGSGSGGSGSGGSG SEVQLVESGGGVVQPGRSLRLSCSASGFDFTTYWMSWVRQAPGKGLEWIGEIHPDSSTI NYAPSLKDRFTISRDNAKNTLFLQMDSLRPEDTGVYFCASLYFGFPWFAYWGQGTPVT VSS (SEQ ID NO: 381). An exemplary nucleic acid sequence encoding SEQ ID NO: 381 is GACATCCAGCTGACACAGAGCCCTAGCAGCCTGTCTGCCTCTGTGGGCGACAGAGT GACCATCACATGCAAGGCCTCTCAGGACGTGGGCACAAGCGTGGCATGGTATCAGC AGAAGCCTGGCAAGGCCCCTAAGCTGCTGATCTACTGGACCAGCACCAGACACACA GGCGTGCCCAGCAGATTTTCTGGCAGCGGCTCTGGCACCGACTTCACCTTCACCATA AGCAGCCTGCAGCCTGAGGATATCGCCACCTACTACTGCCAGCAGTACAGCCTGTA CAGAAGCTTCGGCCAGGGCACCAAGGTGGAAATCAAAGGCGGATCTGGAAGCGGC GGTTCTGGATCTGGTGGAAGCGGATCTGAGGTGCAGCTGGTGGAATCTGGTGGCGG AGTTGTGCAGCCTGGCAGATCTCTGAGACTGAGCTGTAGCGCCAGCGGCTTCGATTT CACCACCTACTGGATGAGCTGGGTCCGACAGGCCCCTGGCAAAGGACTGGAATGGA TCGGCGAGATTCACCCCGACAGCAGCACCATCAATTACGCCCCTAGCCTGAAGGAC CGGTTCACCATCTCCAGAGACAACGCCAAGAATACCCTGTTCCTGCAGATGGACAG CCTCCGGCCTGAAGATACCGGCGTGTACTTTTGCGCCAGCCTGTATTTCGGCTTCCC TTGGTTTGCCTACTGGGGCCAGGGAACACCTGTGACCGTTAGCTCT (SEQ ID NO: 380). In certain embodiments, a nucleic acid encoding SEQ ID NO: 381 comprises a sequence that is at least 90%, at least 91%, at least 92%, at least 93%, at least 94%, at least 95%, at least 96%, at least 97%, at least 98%, or at least 99% identical to SEQ ID NO: 380.

In some embodiments, the antigen-binding domain that binds to CEACAM5 includes a VH region. In some embodiments, the VH region has an amino acid sequence with at least 90%, at least 91%, at least 92%, at least 93%, at least 94%, at least 95%, at least 96%, at least 97%, at least 98%, at least 99%, or 100% identity to the amino acid sequence of EVQLVESGGGVVQPGRSLRLSCSASGFDFTTYWMSWVRQAPGKGLEWIGEIHPDSSTINYAPSL KDRFTISRDNAKNTLFLQMDSLRPEDTGVYFCASLYFGFPWFAYWGQGTPVTVSS (SEQ ID NO: 425). In some embodiments, the VH region has the amino acid sequence EVQLVESGGGVVQPGRSLRLSCSASGFDFTTYWMSWVRQAPGKGLEWIGEIHPDSSTINYAPSL KDRFTISRDNAKNTLFLQMDSLRPEDTGVYFCASLYFGFPWFAYWGQGTPVTVSS (SEQ ID NO: 425). In some embodiments, the antigen-binding domain that binds to CEACAM5 includes a VL region. In some embodiments, the VL region has an amino acid sequence with at least 90%, at least 91%, at least 92%, at least 93%, at least 94%, at least 95%, at least 96%, at least 97%, at least 98%, at least 99%, or 100% identity to the amino acid sequence of DIQMTQTTSSLSASLGDRVTISCRTSQDIGNYLNWYQQKPDGTVKLLIYYTSRLHSGVPSRFSGS GSGTDYSLTISNLEQEDIATYFCQQGKSLPRTFGGGTKLEI (SEQ ID NO: 424). In some embodiments, the VL region has the amino acid sequence DIQMTQTTSSLSASLGDRVTISCRTSQDIGNYLNWYQQKPDGTVKLLIYYTSRLHSGVPSRFSGS GSGTDYSLTISNLEQEDIATYFCQQGKSLPRTFGGGTKLEI (SEQ ID NO: 424). In some embodiments, the VL region has an amino acid sequence with at least 90%, at least 91%, at least 92%, at least 93%, at least 94%, at least 95%, at least 96%, at least 97%, at least 98%, at least 99%, or 100% identity to DIQLTQSPSSLSASVGDRVTITCKASQDVGTSVAWYQQKPGKAPKLLIYWTSTRHTGVPSRFSGS GSGTDFTFTISSLQPEDIATYYCQQYSLYRSFGQGTKVEIK (SEQ ID NO: 426). In some embodiments, the VL region has the amino acid sequence DIQLTQSPSSLSASVGDRVTITCKASQDVGTSVAWYQQKPGKAPKLLIYWTSTRHTGVPSRFSGS GSGTDFTFTISSLQPEDIATYYCQQYSLYRSFGQGTKVEIK (SEQ ID NO: 426). In some embodiments, the antigen-binding domain that binds to CEACAM5 includes a VH region and a VL region. In some embodiments, the antigen-binding domain that binds to CEACAM5 includes a CDR-H1, a CDR-H2, and a CDR-H3 from a VH region that comprises the sequence EVQLVESGGGVVQPGRSLRLSCSASGFDFTTYWMSWVRQAPGKGLEWIGEIHPDSSTINYAPSL KDRFTISRDNAKNTLFLQMDSLRPEDTGVYFCASLYFGFPWFAYWGQGTPVTVSS (SEQ ID NO: 425), and a CDR-L1, a CDR-L2, and a CDR-:3 from a VL region that comprises the sequence

(SEQ ID NO: 424)
DIQMTQTTSSLSASLGDRVTISCRTSQDIGNYLNWYQQKPDGTVKLLIY
YTSRLHSGVPSRFSGSGSGTDYSLTISNLEQEDIATYFCQQGKSLPRTF
GGGTKLEI
or
(SEQ ID NO: 426)
DIQLTQSPSSLSASVGDRVTITCKASQDVGTSVAWYQQKPGKAPKLLIY
WTSTRHTGVPSRFSGSGSGTDFTFTISSLQPEDIATYYCQQYSLYRSFG
QGTKVEIK

Additional antigen binding domains which may be used in the aCAR are described in Table 6D below.

TABLE 6D
Exemplary solid tumor antigen binders

| Solid Tumor | Antibody | |
|---|---|---|
| Antigen | Clone | Clone Sequences |
| CEACAM1 | MRG1 | VH: |
| | | QVQLQQSGAELVRPGTSVKVSCKASGYAFTNNLIEWVKQRPGQG |
| | | LEWIGVINPGSGDTNYNEKFKGKATLTADKSSNTAYMQLSSLTSD |
| | | DSAVYFCARGDYYGGFAVDYWGQGTSVTVSS (SEQ ID NO: 423) |
| | | VL: |
| | | DIQMTQTTSSLSASLGDRVTISCRTSQDIGNYLNWYQQKPDGTVK |
| | | LLIYYTSRLHSGVPSRFSGSGSGTDYSLTISNLEQEDIATYFCQQGK |
| | | SLPRTFGGGTKLEI (SEQ ID NO: 424) |
| CEACAM5 | Labetuzumab | VH: |
| | (i.e., hMN14) | EVQLVESGGGVVQPGRSLRLSCSASGFDFTTYWMSWVRQAPGKG |
| | | LEWIGEIHPDSSTINYAPSLKDRFTISRDNAKNTLFLQMDSLRPEDT |
| | | GVYFCASLYFGFPWFAYWGQGTPVTVSS (SEQ ID NO: 425) |
| | | VL: |
| | | DIQLTQSPSSLSASVGDRVTITCKASQDVGTSVAWYQQKPGKAPK |
| | | LLIYWTSTRHTGVPSRFSGSGSGTDFTFTISSLQPEDIATYYCQQYS |
| | | LYRSFGQGTKVEIK (SEQ ID NO: 426) |
| CEACAM5 | Cibisatamab | HC: |
| | | QVQLVQSGAEVKKPGASVKVSCKASGYTFTEFGMNWVRQAPGQ |
| | | GLEWMGWINTKTGEATYVEEFKGRVTFTTDTSTSTAYMELRSLR |
| | | SDDTAVYYCARWDFAYYVEAMDYWGQGTTVTVSSASTKGPSVF |
| | | PLAPSSKSTSGGTAALGCLVKDYFPEPVTVSWNSGALTSGVHTFP |
| | | AVLQSSGLYSLSSVVTVPSSSLGTQTYICNVNHKPSNTKVDKKVE |
| | | PKSCDKTHTCPPCPAPEAAGGPSVFLFPPKPKDTLMISRTPEVTCV |
| | | VVDVSHEDPEVKFNWYVDGVEVHNAKTKPREEQYNSTYRVVSV |
| | | LTVLHQDWLNGKEYKCKVSNKALGAPIEKTISKAKGQPREPQVC |
| | | TLPPSRDELTKNQVSLSCAVKGFYPSDIAVEWESNGQPENNYKTT |
| | | PPVLDSDGSFFLVSKLTVDKSRWQQGNVFSCSVMHEALHNHYTQ |
| | | KSLSLS (SEQ ID NO: 427) |
| | | LC |
| | | DIQMTQSPSSLSASVGDRVTITCKASAAVGTYVAWYQQKPGKAP |
| | | KLLIYSASYRKRGVPSRFSGSGSGTDFTLTISSLQPEDFATYYCHQ |
| | | YYTYPLFTFGQGTKLEIKRTVAAPSVFIFPPSDEQLKSGTASVVCLL |
| | | NNFYPREAKVQWKVDNALQSGNSQESVTEQDSKDSTYSLSSTLT |
| | | LSKADYEKHKVYAEVTHQGLSSPVTKSFNRGEC (SEQ ID NO: |
| | | 428) |
| CEACAM5 | Tusamitamab | HC: |
| | | EVQLQESGPGLVKPGGSLSLSCAASGFVFSSYDMSWVRQTPERGL |
| | | EWVAYISSGGGITYAPSTVKGRFTVSRDNAKNTLYLQMNSLTSED |
| | | TAVYYCAAHYFGSSGPFAYWGQGTLVTVSSASTKGPSVFPLAPSS |
| | | KSTSGGTAALGCLVKDYFPEPVTVSWNSGALTSGVHTFPAVLQSS |
| | | GLYSLSSVVTVPSSSLGTQTYICNVNHKPSNTKVDKKVEPKSCDK |
| | | THTCPPCPAPELLGGPSVFLFPPKPKDTLMISRTPEVTCVVVDVSH |
| | | EDPEVKFNWYVDGVEVHNAKTKPREEQYNSTYRVVSVLTVLHQ |
| | | DWLNGKEYKCKVSNKALPAPIEKTISKAKGQPREPQVYTLPPSRD |
| | | ELTKNQVSLTCLVKGFYPSDIAVEWESNGQPENNYKTTPPVLDSD |
| | | GSFFLYSKLTVDKSRWQQGNVFSCSVMHEALHNHYTQKSLSLSP |
| | | (SEQ ID NO: 429) |
| | | LC |
| | | DIQMTQSPASLSASVGDRVTITCRASENIFSYLAWYQQKPGKSPKL |
| | | LVYNTRTLAEGVPSRFSGSGSGTDFSLTISSLQPEDFATYYCQHHY |
| | | GTPFTFGSGTKLEIKRTVAAPSVFIFPPSDEQLKSGTASVVCLLNNF |
| | | YPREAKVQWKVDNALQSGNSQESVTEQDSKDSTYSLSSTLTLSK |
| | | ADYEKHKVYACEVTHQGLSSPVTKSFNRGEC (SEQ ID NO: 430) |
| CEACAM5 | BW431/26 | VH: |
| | | QLQESGPGLVRPSQTLSLTCTVSGFTISSGYSWHWVRQPPGRGLE |
| | | WIGYIQYSGITNYNPSLKSRVTMLVDTSKNQFSLRLSSVTAADTA |
| | | VYYCAREDYDYHWYFDVWGQGSLVTVSS (SEQ ID NO: 431) |
| | | VL: |
| | | GVHSDIQMTQSPSSLSASVGDRVTITCSTSSSVSYMHWYQQKPGK |
| | | APKLLIYSTSNLASGVPSRFSGSGSGTDFTFTISSLQPEDIATYYCH |
| | | QWSSYPTFGQGTKVEIKR (SEQ ID NO: 432) |
| CEACAM5 | A5B7 | VH: |
| | | MDFQVQIFSFLLISASVIMSRGQTVLSQSPAILSASPGEKVTMTCRA |
| | | SSSVTYIHWYQQKPGSSPKSWIYATSNLASGVPARFSGSGSGTSYS |
| | | LTISRVEAEDAATYYCQHWSSKPPTFGGGTKLEIKR (SEQ ID NO: |
| | | 433) |
| | | VL: |
| | | MDFQVQIFSFLLISASVIMSRGQTVLSQSPAILSASPGEKVTMTCRA |
| | | SSSVTYIHWYQQKPGSSPARFSGSGTSYSLTISRVEAEDAATYYCQ |
| | | HWSSKPPTFGGGTKLEIKR (SEQ ID NO: 434) |
| CEACAM5 | MFE23 | VH: |
| | | ETVIKYLLPTAAAGLLLLAAQPAMAQVKLQQSGAELVRSGTSVKLSC |
| | | TASGFNIKDSYMHWLRQGPEQGLEWIGWIDPENGDTEYAPKFQGKA |
| | | TFTTDTSSNTAYLQLSSLTSEDTAVYYCNEGTPTGPYYFDYWGQGTT |
| | | VTVSS (SEQ ID NO: 435) |
| | | VL: |
| | | ENVLTQSPAIMSASPGEKVTITCSASSSVSYMHWFQQKPGTSPKLWIY |
| | | STSNLASGVPARFSGSGSGTSYSLTISRMEAEDAATYYCQQRSSYPLT |
| | | FGAGTKLELKRAA (SEQ ID NO: 436) |
| CEACAM5 | hMFE23 | VH: |
| | | QVKLEQSGAEVVKPGASVKLSCKASGFNIKDSYMHWLRQGPGQRLE |
| | | WIGWIDPENGDTEYAPKFQGKATFTTDTSANTAYLGLSSLRPEDTAV |
| | | YYCNEGTPTGPYYFDYWGQGTLVTVSS (SEQ ID NO: 437) |
| | | VL: |
| | | ENVLTQSPSSMSVSVGDRVTIACSASSSVPYMHWLQQKPGKSPKLLI |
| | | YLTSNLASGVPSRFSGSGSGTDYSLTISSVQPEDAATYYCQQRSSYPL |
| | | TFGGGTKLEIK (SEQ ID NO: 438) |
| Glycosylated | FM4 (also | VH: |
| CEACAM5 | referred to | EVKLVESGGGLVQPGGSLRLSCSISGFTFTDYYMNWVRQSPGKALE |
| | as “MG7”) | WLGFIRNKVNGDTTEYSASVKGRFTISRDISQSILYLQMNTLRTEDSA |
| | | TYYCARDKGIAYYFDYWGQGTTLTVSS (SEQ ID NO: 439) |
| | | VL: |
| | | QIVLSQSPAILFASPGEKVTMTCRASSSVSYIHWYQQKPGSSPKPWIH |
| | | GTSNLASGVPARFSGSGSGTSYSLTISRMEAEDAATYYCQQWSSNLS |
| | | TFGGGTKLEIK (SEQ ID NO: 440) |
| CEACAM6 | Tinurilimab | HC: |
| | | QVTLRESGPALVKPTQTLTLTCTFSGFSLSTYGIGVGWIRQPPGKA |
| | | LEWLAHIWWNDNKYYSTSLKTRLTISKDTSKNQVVLTMTNMDP |
| | | VDTATYYCARISLPYFDYWGQGTTLTVSSASTKGPSVFPLAPCSRS |
| | | TSESTAALGCLVKDYFPEPVTVSWNSGALTSGVHTFPAVLQSSGL |
| | | YSLSSVVTVPSSNFGTQTYTCNVDHKPSNTKVDKTVERKCCVECP |
| | | PCPAPPVAGPSVFLFPPKPKDTLMISRTPEVTCVVVDVSHEDPEVQ |
| | | FNWYVDGVEVHNAKTKPREEQFNSTFRVVSVLTVVHQDWLNGK |
| | | EYKCKVSNKGLPAPIEKTISKTKGQPREPQVYTLPPSREEMTKNQV |
| | | SLTCLVKGFYPSDIAVEWESNGQPENNYKTTPPMLDSDGSFFLYS |
| | | KLTVDKSRWQQGNVFSCSVMHEALHNHYTQKSLSLSPG (SEQ ID |
| | | NO: 441) |
| | | LC: |
| | | DIQLTQSPSFLSASVGDRVTITCKASQNVGTAVAWYQQKPGKAPK |
| | | LLIYSASNRYTGVPSRFSGSGSGTEFTLTISSLQPEDFATYYCQQYS |
| | | SYPLTFGGGTKVEIKRTVAAPSVFIFPPSDEQLKSGTASVVCLLNN |
| | | FYPREAKVQWKVDNALQSGNSQESVTEQDSKDSTYSLSSTLTLSK |
| | | ADYEKHKVYACEVTHQGLSSPVTKSFNRGEC (SEQ ID NO: 442) |

In some embodiments, the aCAR that binds CEACAM5 comprises the amino acid sequence of MALPVTALLLPLALLLHAARPDIQLTQSPSSLSASVGDRVTITCKASQDVGTSVAWYQQ KPGKAPKLLIYWTSTRHTGVPSRFSGSGSGTDFTFTISSLQPEDIATYYCQQYSLYRSFGQ GTKVEIKGGSGSGGSGSGGSGSEVQLVESGGGVVQPGRSLRLSCSASGFDFTTYWMSW VRQAPGKGLEWIGEIHPDSSTINYAPSLKDRFTISRDNAKNTLFLQMDSLRPEDTGVYFC ASLYFGFPWFAYWGQGTPVTVSSEQKLISEEDLNGAATTTPAPRPPTPAPTIALQPLSLR PEACRPAAGGAVHTRGLDFACDFWVLVVVGGVLACYSLLVTVAFIIFWVRSKRSRLLH SDYMNMTPRRPGPTRKHYQPYAPPRDFAAYRSRVKFSRSADAPAYKQGQNQLYNELN LGRREEYDVLDKRRGRDPEMGGKPRRKNPQEGLYNELQKDKMAEAYSEIGMKGERRR GKGHDGLYQGLSTATKDTYDALHMQALPPR (SEQ ID NO: 362). An exemplary nucleic acid sequence encoding SEQ ID NO: 362 is

(SEQ ID NO: 373)
ATGGCCCTGCCTGTGACAGCTCTGCTGCTGCCTCTGGCCCTGCTGCTGCATGCTGCT
AGACCTGACATCCAGCTGACACAGAGCCCTAGCAGCCTGTCTGCCTCTGTGGGCGA
CAGAGTGACCATCACATGCAAGGCCTCTCAGGACGTGGGCACAAGCGTGGCATGGT
ATCAGCAGAAGCCTGGCAAGGCCCCTAAGCTGCTGATCTACTGGACCAGCACCAGA
CACACAGGCGTGCCCAGCAGATTTTCTGGCAGCGGCTCTGGCACCGACTTCACCTTC
ACCATAAGCAGCCTGCAGCCTGAGGATATCGCCACCTACTACTGCCAGCAGTACAG
CCTGTACAGAAGCTTCGGCCAGGGCACCAAGGTGGAAATCAAAGGCGGATCTGGA
AGCGGCGGTTCTGGATCTGGTGGAAGCGGATCTGAGGTGCAGCTGGTGGAATCTGG
TGGCGGAGTTGTGCAGCCTGGCAGATCTCTGAGACTGAGCTGTAGCGCCAGCGGCT
TCGATTTCACCACCTACTGGATGAGCTGGGTCCGACAGGCCCCTGGCAAAGGACTG
GAATGGATCGGCGAGATTCACCCCGACAGCAGCACCATCAATTACGCCCCTAGCCT
GAAGGACCGGTTCACCATCTCCAGAGACAACGCCAAGAATACCCTGTTCCTGCAGA
TGGACAGCCTCCGGCCTGAAGATACCGGCGTGTACTTTTGCGCCAGCCTGTATTTCG
GCTTCCCTTGGTTTGCCTACTGGGGCCAGGGAACACCTGTGACCGTTAGCTCTGAAC
AAAAACTCATCTCAGAAGAAGATCTGAATGGGGCCGCAACCACGACGCCAGCGCC
GCGACCACCAACACCGGCGCCCACCATCGCGTTGCAGCCCCTGTCCCTGCGCCCAG
AGGCGTGCCGGCCAGCGGCGGGGGGCGCAGTGCACACGAGGGGGCTGGACTTCGC
CTGTGATTTTTGGGTGCTGGTGGTGGTTGGTGGAGTCCTGGCTTGCTATAGCTTGCT
AGTAACAGTGGCCTTTATTATTTTCTGGGTGAGGAGTAAGAGGAGCAGGCTCCTGC
ACAGTGACTACATGAACATGACTCCCCGCCGCCCCGGGCCCACCCGCAAGCATTAC
CAGCCCTATGCCCCACCACGCGACTTCGCAGCCTATCGCTCCAGAGTGAAGTTCAGC
AGGAGCGCAGACGCCCCCGCGTACAAGCAGGGCCAGAACCAGCTCTATAACGAGC
TCAATCTAGGACGAAGAGAGGAGTACGATGTTTTGGACAAGAGACGTGGCCGGGA
CCCTGAGATGGGGGGAAAGCCGAGAAGGAAGAACCCTCAGGAAGGCCTGTACAAT
GAACTGCAGAAAGATAAGATGGCGGAGGCCTACAGTGAGATTGGGATGAAAGGCG
AGCGCCGGAGGGGCAAGGGGCACGATGGCCTTTACCAGGGTCTCAGTACAGCCACC
AAGGACACCTACGACGCCCTTCACATGCAGGCCCTGCCCCCTCGC.

In certain embodiments, a nucleic acid encoding SEQ ID NO: 362 comprises a sequence that is at least 90%, at least 91%, at least 92%, at least 93%, at least 94%, at least 95%, at least 96%, at least 97%, at least 98%, or at least 99% identical to SEQ ID NO: 373.

In some embodiments, the aCAR that binds CEACAM5 comprises the amino acid sequence of MALPVTALLLPLALLLHAARPDIQLTQSPSSLSASVGDRVTITCKASQDVGTSVAWYQQ KPGKAPKLLIYWTSTRHTGVPSRFSGSGSGTDFTFTISSLQPEDIATYYCQQYSLYRSFGQ GTKVEIKGGSGSGGSGSGGSGSEVQLVESGGGVVQPGRSLRLSCSASGFDFTTYWMSW VRQAPGKGLEWIGEIHPDSSTINYAPSLKDRFTISRDNAKNTLFLQMDSLRPEDTGVYFC ASLYFGFPWFAYWGQGTPVTVSSEQKLISEEDLNGAATTTPAPRPPTPAPTIALQPLSLR PEACRPAAGGAVHTRGLDFACDVAAILGLGLVLGLLGPLAILLALYLLRRDQRLPPDAH KPPGGGSFRTPIQEEQADAHSTLAKIRVKFSRSADAPAYKQGQNQLYNELNLGRREEYD VLDKRRGRDPEMGGKPRRKNPQEGLYNELQKDKMAEAYSEIGMKGERRRGKGHDGL YQGLSTATKDTYDALHMQALPPR (SEQ ID NO: 363). An exemplary nucleic acid sequence encoding SEQ ID NO: 363 is ATGGCCCTGCCTGTGACAGCTCTGCTGCTGCCTCTGGCCCTGCTGCTGCATGCTGCT AGACCTGACATCCAGCTGACACAGAGCCCTAGCAGCCTGTCTGCCTCTGTGGGCGA CAGAGTGACCATCACATGCAAGGCCTCTCAGGACGTGGGCACAAGCGTGGCATGGT ATCAGCAGAAGCCTGGCAAGGCCCCTAAGCTGCTGATCTACTGGACCAGCACCAGA CACACAGGCGTGCCCAGCAGATTTTCTGGCAGCGGCTCTGGCACCGACTTCACCTTC ACCATAAGCAGCCTGCAGCCTGAGGATATCGCCACCTACTACTGCCAGCAGTACAG CCTGTACAGAAGCTTCGGCCAGGGCACCAAGGTGGAAATCAAAGGCGGATCTGGA AGCGGCGGTTCTGGATCTGGTGGAAGCGGATCTGAGGTGCAGCTGGTGGAATCTGG TGGCGGAGTTGTGCAGCCTGGCAGATCTCTGAGACTGAGCTGTAGCGCCAGCGGCT TCGATTTCACCACCTACTGGATGAGCTGGGTCCGACAGGCCCCTGGCAAAGGACTG GAATGGATCGGCGAGATTCACCCCGACAGCAGCACCATCAATTACGCCCCTAGCCT GAAGGACCGGTTCACCATCTCCAGAGACAACGCCAAGAATACCCTGTTCCTGCAGA TGGACAGCCTCCGGCCTGAAGATACCGGCGTGTACTTTTGCGCCAGCCTGTATTTCG GCTTCCCTTGGTTTGCCTACTGGGGCCAGGGAACACCTGTGACCGTTAGCTCTGAAC AAAAACTCATCTCAGAAGAAGATCTGAATGGGGCCGCAACCACGACGCCAGCGCC GCGACCACCAACACCGGCGCCCACCATCGCGTTGCAGCCCCTGTCCCTGCGCCCAG AGGCGTGCCGGCCAGCGGCGGGGGGCGCAGTGCACACGAGGGGGCTGGACTTCGC CTGTGATGTGGCCGCCATTCTTGGACTGGGACTTGTTCTGGGACTGCTGGGACCTCT GGCCATTCTGCTGGCTCTGTACCTGCTGAGAAGGGACCAGAGACTGCCTCCTGACG CTCACAAACCTCCAGGCGGAGGCAGCTTCAGAACCCCTATCCAAGAGGAACAGGCT GACGCCCACAGCACCCTGGCCAAGATCAGAGTGAAGTTCAGCAGGAGCGCAGACG CCCCCGCGTACAAGCAGGGCCAGAACCAGCTCTATAACGAGCTCAATCTAGGACGA AGAGAGGAGTACGATGTTTTGGACAAGAGACGTGGCCGGGACCCTGAGATGGGGG GAAAGCCGAGAAGGAAGAACCCTCAGGAAGGCCTGTACAATGAACTGCAGAAAGA TAAGATGGCGGAGGCCTACAGTGAGATTGGGATGAAAGGCGAGCGCCGGAGGGGC AAGGGGCACGATGGCCTTTACCAGGGTCTCAGTACAGCCACCAAGGACACCTACGA CGCCCTTCACATGCAGGCCCTGCCCCCTCGC (SEQ ID NO: 374). In certain embodiments, a nucleic acid encoding SEQ ID NO: 363 comprises a sequence that is at least 90%, at least 91%, at least 92%, at least 93%, at least 94%, at least 95%, at least 96%, at least 97%, at least 98%, or at least 99% identical to SEQ ID NO: 374.

In some embodiments, the aCAR that binds CEACAM5 comprises the amino acid sequence of MALPVTALLLPLALLLHAARPDIQLTQSPSSLSASVGDRVTITCKASQDVGTSVAWYQQ KPGKAPKLLIYWTSTRHTGVPSRFSGSGSGTDFTFTISSLQPEDIATYYCQQYSLYRSFGQ GTKVEIKGGSGSGGSGSGGSGSEVQLVESGGGVVQPGRSLRLSCSASGFDFTTYWMSW VRQAPGKGLEWIGEIHPDSSTINYAPSLKDRFTISRDNAKNTLFLQMDSLRPEDTGVYFC ASLYFGFPWFAYWGQGTPVTVSSEQKLISEEDLNGAATTTPAPRPPTPAPTIALQPLSLR PEACRPAAGGAVHTRGLDFACDIYIWAPLAGTCGVLLLSLVITKRGRKKLLYIFKQPFM RPVQTTQEEDGCSCRFPEEEEGGCELRVKFSRSADAPAYKQGQNQLYNELNLGRREEY DVLDKRRGRDPEMGGKPRRKNPQEGLYNELQKDKMAEAYSEIGMKGERRRGKGHDG LYQGLSTATKDTYDALHMQALPPR (SEQ ID NO: 364). An exemplary nucleic acid sequence encoding SEQ ID NO: 364 is ATGGCCCTGCCTGTGACAGCTCTGCTGCTGCCTCTGGCCCTGCTGCTGCATGCTGCT AGACCTGACATCCAGCTGACACAGAGCCCTAGCAGCCTGTCTGCCTCTGTGGGCGA CAGAGTGACCATCACATGCAAGGCCTCTCAGGACGTGGGCACAAGCGTGGCATGGT ATCAGCAGAAGCCTGGCAAGGCCCCTAAGCTGCTGATCTACTGGACCAGCACCAGA CACACAGGCGTGCCCAGCAGATTTTCTGGCAGCGGCTCTGGCACCGACTTCACCTTC ACCATAAGCAGCCTGCAGCCTGAGGATATCGCCACCTACTACTGCCAGCAGTACAG CCTGTACAGAAGCTTCGGCCAGGGCACCAAGGTGGAAATCAAAGGCGGATCTGGA AGCGGCGGTTCTGGATCTGGTGGAAGCGGATCTGAGGTGCAGCTGGTGGAATCTGG TGGCGGAGTTGTGCAGCCTGGCAGATCTCTGAGACTGAGCTGTAGCGCCAGCGGCT TCGATTTCACCACCTACTGGATGAGCTGGGTCCGACAGGCCCCTGGCAAAGGACTG GAATGGATCGGCGAGATTCACCCCGACAGCAGCACCATCAATTACGCCCCTAGCCT GAAGGACCGGTTCACCATCTCCAGAGACAACGCCAAGAATACCCTGTTCCTGCAGA TGGACAGCCTCCGGCCTGAAGATACCGGCGTGTACTTTTGCGCCAGCCTGTATTTCG GCTTCCCTTGGTTTGCCTACTGGGGCCAGGGAACACCTGTGACCGTTAGCTCTGAAC AAAAACTCATCTCAGAAGAAGATCTGAATGGGGCCGCAACCACGACGCCAGCGCC GCGACCACCAACACCGGCGCCCACCATCGCGTTGCAGCCCCTGTCCCTGCGCCCAG AGGCGTGCCGGCCAGCGGCGGGGGGCGCAGTGCACACGAGGGGGCTGGACTTCGC CTGTGATATCTACATCTGGGCCCCTCTGGCTGGAACATGCGGAGTGTTGCTGCTGAG CCTGGTCATCACCAAGCGGGGCAGAAAGAAGCTGCTGTACATCTTCAAGCAGCCCT TCATGCGGCCCGTGCAGACCACACAAGAGGAAGATGGCTGCTCCTGCAGATTCCCC GAGGAAGAAGAAGGCGGCTGCGAGCTGAGAGTGAAGTTCAGCAGGAGCGCAGACG CCCCCGCGTACAAGCAGGGCCAGAACCAGCTCTATAACGAGCTCAATCTAGGACGA AGAGAGGAGTACGATGTTTTGGACAAGAGACGTGGCCGGGACCCTGAGATGGGGG GAAAGCCGAGAAGGAAGAACCCTCAGGAAGGCCTGTACAATGAACTGCAGAAAGA TAAGATGGCGGAGGCCTACAGTGAGATTGGGATGAAAGGCGAGCGCCGGAGGGGC AAGGGGCACGATGGCCTTTACCAGGGTCTCAGTACAGCCACCAAGGACACCTACGA CGCCCTTCACATGCAGGCCCTGCCCCCTCGC (SEQ ID NO: 375). In certain embodiments, a nucleic acid encoding SEQ ID NO: 364 comprises a sequence that is at least 90%, at least 91%, at least 92%, at least 93%, at least 94%, at least 95%, at least 96%, at least 97%, at least 98%, or at least 99% identical to SEQ ID NO: 375.

In some embodiments, the aCAR that binds CEACAM5 comprises the amino acid sequence of MALPVTALLLPLALLLHAARPDIQLTQSPSSLSASVGDRVTITCKASQDVGTSVAWYQQ KPGKAPKLLIYWTSTRHTGVPSRFSGSGSGTDFTFTISSLQPEDIATYYCQQYSLYRSFGQ GTKVEIKGGSGSGGSGSGGSGSEVQLVESGGGVVQPGRSLRLSCSASGFDFTTYWMSW VRQAPGKGLEWIGEIHPDSSTINYAPSLKDRFTISRDNAKNTLFLQMDSLRPEDTGVYFC ASLYFGFPWFAYWGQGTPVTVSSEQKLISEEDLNGAATTTPAPRPPTPAPTIALQPLSLR PEACRPAAGGAVHTRGLDFACDFWVLVVVGGVLACYSLLVTVAFIIFWVRSKRSRLLH SDYMNMTPRRPGPTRKHYQPYAPPRDFAAYRSWRRKRKEKQSETSPKEFLTIYEDVKD LKTRRNHEQEQTFPGGGSTIYSMIQSQSSAPTSQEPAYTLYSLIQPSRKSGSRKRNHSPSF NSTIYEVIGKSQPKAQNPARLSRKELENFDVYSRVKFSRSADAPAYKQGQNQLYNELNL GRREEYDVLDKRRGRDPEMGGKPRRKNPQEGLYNELQKDKMAEAYSEIGMKGERRR GKGHDGLYQGLSTATKDTYDALHMQALPPR (SEQ ID NO: 365). An exemplary nucleic acid sequence encoding SEQ ID NO: 365 is ATGGCCCTGCCTGTGACAGCTCTGCTGCTGCCTCTGGCCCTGCTGCTGCATGCTGCT AGACCTGACATCCAGCTGACACAGAGCCCTAGCAGCCTGTCTGCCTCTGTGGGCGA CAGAGTGACCATCACATGCAAGGCCTCTCAGGACGTGGGCACAAGCGTGGCATGGT ATCAGCAGAAGCCTGGCAAGGCCCCTAAGCTGCTGATCTACTGGACCAGCACCAGA CACACAGGCGTGCCCAGCAGATTTTCTGGCAGCGGCTCTGGCACCGACTTCACCTTC ACCATAAGCAGCCTGCAGCCTGAGGATATCGCCACCTACTACTGCCAGCAGTACAG CCTGTACAGAAGCTTCGGCCAGGGCACCAAGGTGGAAATCAAAGGCGGATCTGGA AGCGGCGGTTCTGGATCTGGTGGAAGCGGATCTGAGGTGCAGCTGGTGGAATCTGG TGGCGGAGTTGTGCAGCCTGGCAGATCTCTGAGACTGAGCTGTAGCGCCAGCGGCT TCGATTTCACCACCTACTGGATGAGCTGGGTCCGACAGGCCCCTGGCAAAGGACTG GAATGGATCGGCGAGATTCACCCCGACAGCAGCACCATCAATTACGCCCCTAGCCT GAAGGACCGGTTCACCATCTCCAGAGACAACGCCAAGAATACCCTGTTCCTGCAGA TGGACAGCCTCCGGCCTGAAGATACCGGCGTGTACTTTTGCGCCAGCCTGTATTTCG GCTTCCCTTGGTTTGCCTACTGGGGCCAGGGAACACCTGTGACCGTTAGCTCTGAAC AAAAACTCATCTCAGAAGAAGATCTGAATGGGGCCGCAACCACGACGCCAGCGCC GCGACCACCAACACCGGCGCCCACCATCGCGTTGCAGCCCCTGTCCCTGCGCCCAG AGGCGTGCCGGCCAGCGGCGGGGGGCGCAGTGCACACGAGGGGGCTGGACTTCGC CTGTGATTTTTGGGTGCTGGTGGTGGTTGGTGGAGTCCTGGCTTGCTATAGCTTGCT AGTAACAGTGGCCTTTATTATTTTCTGGGTGAGGAGTAAGAGGAGCAGGCTCCTGC ACAGTGACTACATGAACATGACTCCCCGCCGCCCCGGGCCCACCCGCAAGCATTAC CAGCCCTATGCCCCACCACGCGACTTCGCAGCCTATCGCTCCTGGCGCCGCAAGCG GAAAGAGAAGCAGTCTGAGACAAGCCCCAAAGAGTTCCTGACCATCTACGAGGAC GTGAAGGACCTGAAAACCCGGCGGAACCACGAGCAAGAGCAGACCTTTCCTGGCG GCGGAAGCACCATCTACAGCATGATCCAGAGCCAGTCTAGCGCCCCTACCAGCCAA GAGCCTGCCTACACACTGTACTCCCTGATCCAGCCTAGCAGAAAGAGCGGCAGCCG GAAGAGAAATCACAGCCCCAGCTTCAACAGCACGATCTACGAAGTGATCGGCAAG AGCCAGCCAAAGGCTCAGAACCCTGCCAGGCTGAGCCGGAAAGAGCTGGAAAACT TCGACGTGTACAGCAGAGTGAAGTTCAGCAGGAGCGCAGACGCCCCCGCGTACAAG CAGGGCCAGAACCAGCTCTATAACGAGCTCAATCTAGGACGAAGAGAGGAGTACG ATGTTTTGGACAAGAGACGTGGCCGGGACCCTGAGATGGGGGGAAAGCCGAGAAG GAAGAACCCTCAGGAAGGCCTGTACAATGAACTGCAGAAAGATAAGATGGCGGAG GCCTACAGTGAGATTGGGATGAAAGGCGAGCGCCGGAGGGGCAAGGGGCACGATG GCCTTTACCAGGGTCTCAGTACAGCCACCAAGGACACCTACGACGCCCTTCACATG CAGGCCCTGCCCCCTCGC (SEQ ID NO: 376). In certain embodiments, a nucleic acid encoding SEQ ID NO: 365 comprises a sequence that is at least 90%, at least 91%, at least 92%, at least 93%, at least 94%, at least 95%, at least 96%, at least 97%, at least 98%, or at least 99% identical to SEQ ID NO: 376.

In some embodiments, the aCAR that binds CEACAM5 comprises the amino acid sequence of MALPVTALLLPLALLLHAARPDIQLTQSPSSLSASVGDRVTITCKASQDVGTSVAWYQQ KPGKAPKLLIYWTSTRHTGVPSRFSGSGSGTDFTFTISSLQPEDIATYYCQQYSLYRSFGQ GTKVEIKGGGGSGGGGSGGGGSEVQLVESGGGVVQPGRSLRLSCSASGFDFTTYWMS WVRQAPGKGLEWIGEIHPDSSTINYAPSLKDRFTISRDNAKNTLFLQMDSLRPEDTGVY FCASLYFGFPWFAYWGQGTPVTVSSNGAATTTPAPRPPTPAPTIALQPLSLRPEACRPAA GGAVHTRGLDFACDFWVLVVVGGVLACYSLLVTVAFIIFWVRSKRSRLLHSDYMNMT PRRPGPTRKHYQPYAPPRDFAAYRSRVKFSRSADAPAYKQGQNQLYNELNLGRREEYD VLDKRRGRDPEMGGKPRRKNPQEGLYNELQKDKMAEAYSEIGMKGERRRGKGHDGL YQGLSTATKDTYDALHMQALPPR (SEQ ID NO: 417). An exemplary nucleic acid sequence encoding SEQ ID NO: 417 is

(SEQ ID NO: 418)
ATGGCCCTGCCTGTGACAGCTCTGCTGCTGCCTCTGGCCCTGCTGCTGCATGCTGCT
AGACCTGACATCCAGCTGACACAGAGCCCTAGCAGCCTGTCTGCCTCTGTGGGCGA
CAGAGTGACCATCACATGCAAGGCCTCTCAGGACGTGGGCACAAGCGTGGCATGGT
ATCAGCAGAAGCCTGGCAAGGCCCCTAAGCTGCTGATCTACTGGACCAGCACCAGA
CACACAGGCGTGCCCAGCAGATTTTCTGGCAGCGGCTCTGGCACCGACTTCACCTTC
ACCATAAGCAGCCTGCAGCCTGAGGATATCGCCACCTACTACTGCCAGCAGTACAG
CCTGTACAGAAGCTTCGGCCAGGGCACCAAGGTGGAAATCAAAGGCGGAggTGGAA
GCGGCGGTggTGGATCTGGTGGAgGCGGATCTGAGGTGCAGCTGGTGGAATCTGGTG
GCGGAGTTGTGCAGCCTGGCAGATCTCTGAGACTGAGCTGTAGCGCCAGCGGCTTC
GATTTCACCACCTACTGGATGAGCTGGGTCCGACAGGCCCCTGGCAAAGGACTGGA
ATGGATCGGCGAGATTCACCCCGACAGCAGCACCATCAATTACGCCCCTAGCCTGA
AGGACCGGTTCACCATCTCCAGAGACAACGCCAAGAATACCCTGTTCCTGCAGATG
GACAGCCTCCGGCCTGAAGATACCGGCGTGTACTTTTGCGCCAGCCTGTATTTCGGC
TTCCCTTGGTTTGCCTACTGGGGCCAGGGAACACCTGTGACCGTTAGCTCTAATGGG
GCCGCAACCACGACGCCAGCGCCGCGACCACCAACACCGGCGCCCACCATCGCGTT
GCAGCCCCTGTCCCTGCGCCCAGAGGCGTGCCGGCCAGCGGCGGGGGGCGCAGTGC
ACACGAGGGGGCTGGACTTCGCCTGTGATTTTTGGGTGCTGGTGGTGGTTGGTGGA
GTCCTGGCTTGCTATAGCTTGCTAGTAACAGTGGCCTTTATTATTTTCTGGGTGAGG
AGTAAGAGGAGCAGGCTCCTGCACAGTGACTACATGAACATGACTCCCCGCCGCCC
CGGGCCCACCCGCAAGCATTACCAGCCCTATGCCCCACCACGCGACTTCGCAGCCT
ATCGCTCCAGAGTGAAGTTCAGCAGGAGCGCAGACGCCCCCGCGTACAAGCAGGGC
CAGAACCAGCTCTATAACGAGCTCAATCTAGGACGAAGAGAGGAGTACGATGTTTT
GGACAAGAGACGTGGCCGGGACCCTGAGATGGGGGGAAAGCCGAGAAGGAAGAA
CCCTCAGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCC
CTGCCCCCTCGC.

In some embodiments, the antigen-binding domain of the iCAR binds to VSIG2. In some embodiments, the antigen-binding domain that binds to VSIG2 includes an scFv having an amino acid sequence with at least 90%, at least 91%, at least 92%, at least 93%, at least 94%, at least 95%, at least 96%, at least 97%, at least 98%, at least 99%, or 100% identity to the amino acid sequence of EVQMVESGGDLVKPGGSLKLSCAASGFTFSNSGMSWVRQTPDKRLEWVASISDGGLYT HYPDSVKGRFTISRDNGKSTLYLQMSSLRSEDTAIYYCARQGVRPFFDYWGQGTTLTVS SGSTSGSGKPGSGEGSTKGDIQMTQSPASLSASVGETVTMTCRASENIYSYLAWYQQKQ GKSPQLLVFNAETLPEGVPSRFSGTGSGTHFSLRINSLQPEDFGSYYCQHHYVIPWTFGG GTKLEIK (SEQ ID NO: 379). An exemplary nucleic acid sequence encoding SEQ ID NO: 379 is

(SEQ ID NO: 378)
GAGGTGCAGATGGTTGAGTCTGGCGGCGATCTGGTTAAGCCTGGCGGAAGCCTGAA
GCTGTCTTGTGCCGCCAGCGGCTTCACCTTCAGCAATAGCGGCATGAGCTGGGTCCG
ACAGACCCCTGACAAGAGACTGGAATGGGTCGCCAGCATCTCTGACGGCGGCCTGT
ACACACACTACCCCGATTCTGTGAAGGGCAGATTCACCATCAGCAGAGACAACGGC
AAGAGCACCCTGTACCTGCAGATGAGCAGCCTGAGAAGCGAGGACACCGCCATCTA
CTACTGCGCCAGACAGGGCGTCAGACCCTTCTTCGATTATTGGGGCCAGGGCACCA
CACTGACCGTGTCATCTGGCAGCACAAGCGGCTCTGGAAAACCTGGATCTGGCGAG
GGCTCTACCAAGGGCGACATCCAGATGACACAGTCTCCAGCCAGCCTGTCTGCCTCT
GTGGGAGAGACAGTGACCATGACCTGTCGGGCCAGCGAGAACATCTACAGCTACCT
GGCCTGGTATCAGCAGAAGCAGGGCAAGTCTCCTCAGCTGCTGGTGTTCAACGCCG
AGACACTGCCTGAAGGCGTGCCCAGCAGATTTTCTGGAACAGGCAGCGGCACCCAC
TTCAGCCTGAGAATCAATAGCCTGCAGCCTGAGGACTTCGGCAGCTACTACTGCCA
GCACCACTACGTGATCCCTTGGACCTTTGGCGGAGGCACCAAGCTGGAAATCAAG.

In some embodiments, the iCAR that binds VSIG2 that comprises the amino acid sequence of MALPVTALLLPLALLLHAARPEVQMVESGGDLVKPGGSLKLSCAASGFTFSNSGMSWV RQTPDKRLEWVASISDGGLYTHYPDSVKGRFTISRDNGKSTLYLQMSSLRSEDTAIYYC ARQGVRPFFDYWGQGTTLTVSSGSTSGSGKPGSGEGSTKGDIQMTQSPASLSASVGETV TMTCRASENIYSYLAWYQQKQGKSPQLLVFNAETLPEGVPSRFSGTGSGTHFSLRINSL QPEDFGSYYCQHHYVIPWTFGGGTKLEIKGKPIPNPLLGLDSTNGAATTTPAPRPPTPAP TIALQPLSLRPEACRPAAGGAVHTRGLDFACDVIGILVAVILLLLLLLLLFLILRHRRQGK HWTSTQRKADFQHPAGAVGPEPTDRGLQWRSSPAADAQEENLYAAVKHTQPEDGVE MDTRSPHDEDPQAVTYAEVKHSRPRREMASPPSPLSGEFLDTKDRQAEEDRQMDTEAA ASEAPQDVTYAQLHSLTLRREATEPPPSQEGPSPAVPSIYATLAIH (SEQ ID NO: 366). An exemplary nucleic acid sequence encoding SEQ ID NO: 366 is ATGGCCTTACCAGTGACCGCCTTGCTCCTGCCGCTGGCCTTGCTGCTCCACGCCGCC AGGCCGGAGGTGCAGATGGTTGAGTCTGGCGGCGATCTGGTTAAGCCTGGCGGAAG CCTGAAGCTGTCTTGTGCCGCCAGCGGCTTCACCTTCAGCAATAGCGGCATGAGCTG GGTCCGACAGACCCCTGACAAGAGACTGGAATGGGTCGCCAGCATCTCTGACGGCG GCCTGTACACACACTACCCCGATTCTGTGAAGGGCAGATTCACCATCAGCAGAGAC AACGGCAAGAGCACCCTGTACCTGCAGATGAGCAGCCTGAGAAGCGAGGACACCG CCATCTACTACTGCGCCAGACAGGGCGTCAGACCCTTCTTCGATTATTGGGGCCAGG GCACCACACTGACCGTGTCATCTGGCAGCACAAGCGGCTCTGGAAAACCTGGATCT GGCGAGGGCTCTACCAAGGGCGACATCCAGATGACACAGTCTCCAGCCAGCCTGTC TGCCTCTGTGGGAGAGACAGTGACCATGACCTGTCGGGCCAGCGAGAACATCTACA GCTACCTGGCCTGGTATCAGCAGAAGCAGGGCAAGTCTCCTCAGCTGCTGGTGTTC AACGCCGAGACACTGCCTGAAGGCGTGCCCAGCAGATTTTCTGGAACAGGCAGCGG CACCCACTTCAGCCTGAGAATCAATAGCCTGCAGCCTGAGGACTTCGGCAGCTACT ACTGCCAGCACCACTACGTGATCCCTTGGACCTTTGGCGGAGGCACCAAGCTGGAA ATCAAGGGGAAGCCTATCCCGAACCCTCTGTTGGGTCTCGATAGTACCAATGGGGC CGCAACCACGACGCCAGCGCCGCGACCACCAACACCGGCGCCCACCATCGCGTTGC AGCCCCTGTCCCTGCGCCCAGAGGCGTGCCGGCCAGCGGCGGGGGGCGCAGTGCAC ACGAGGGGGCTGGACTTCGCCTGTGATGTTATAGGGATCCTGGTGGCTGTCATACTC CTCTTGCTCCTCTTGTTGCTGCTTTTTTTGATATTGCGCCACAGACGGCAGGGAAAG CACTGGACTAGTACGCAGAGGAAAGCGGACTTCCAGCATCCCGCAGGAGCCGTGGG GCCTGAACCCACTGATCGCGGCCTTCAATGGAGGTCTAGCCCGGCGGCAGACGCAC AAGAGGAAAACTTGTACGCAGCCGTTAAGCACACCCAACCGGAGGACGGCGTTGA GATGGATACCCGCTCCCCTCACGATGAAGACCCTCAAGCAGTCACTTACGCGGAAG TAAAGCATAGCCGCCCCAGACGGGAAATGGCTAGCCCGCCGTCCCCCCTTAGCGGG GAATTTCTGGACACTAAAGATAGGCAGGCGGAAGAGGACCGCCAAATGGATACAG AGGCGGCGGCAAGTGAAGCACCTCAAGACGTTACTTACGCTCAACTTCACAGCCTT ACCCTCAGGCGAGAAGCGACTGAACCACCCCCTTCCCAAGAAGGGCCAAGCCCAGC GGTTCCTTCTATCTATGCTACTCTTGCTATTCAC (SEQ ID NO: 377). In certain embodiments, a nucleic acid encoding SEQ ID NO: 366 comprises a sequence that is at least 90%, at least 91%, at least 92%, at least 93%, at least 94%, at least 95%, at least 96%, at least 97%, at least 98%, or at least 99% identical to SEQ ID NO: 377.

In some embodiments, the iCAR that binds VSIG2 that comprises the amino acid sequence of MALPVTALLLPLALLLHAARPDIQMTQSPASLSASVGETVTMTCRASENIYSYLAWYQ QKQGKSPQLLVFNAETLPEGVPSRFSGTGSGTHFSLRINSLQPEDFGSYYCQHHYVIPWT FGGGTKLEIKGSTSGSGKPGSGEGSTKGEVQMVESGGDLVKPGGSLKLSCAASGFTFSN SGMSWVRQTPDKRLEWVASISDGGLYTHYPDSVKGRFTISRDNGKSTLYLQMSSLRSE DTAIYYCARQGVRPFFDYWGQGTTLTVSSGKPIPNPLLGLDSTNGAATTTPAPRPPTPAP TIALQPLSLRPEACRPAAGGAVHTRGLDFACDIVVGVVCTLLVALLMAALYLVRIRQKK AQGSTSSTRLHEPEKNAREITQDTNDITYADLNLPKGKKPAPQAAEPNNHTEYASIQTSP QPASEDTLTYADLDMVHLNRTPKQPAPKPEPSFSEYASVQVPRK (SEQ ID NO: 419). An exemplary nucleic acid sequence encoding SEQ ID NO: 419 is

(SEQ ID NO: 420)
ATGGCCTTACCAGTGACCGCCTTGCTCCTGCCGCTGGCCTTGCTGCTCCACGCCGCC
AGGCCGGACATCCAGATGACACAGTCTCCAGCCAGCCTGTCTGCCTCTGTGGGAGA
GACAGTGACCATGACCTGTCGGGCCAGCGAGAACATCTACAGCTACCTGGCCTGGT
ATCAGCAGAAGCAGGGCAAGTCTCCTCAGCTGCTGGTGTTCAACGCCGAGACACTG
CCTGAAGGCGTGCCCAGCAGATTTTCTGGAACAGGCAGCGGCACCCACTTCAGCCT
GAGAATCAATAGCCTGCAGCCTGAGGACTTCGGCAGCTACTACTGCCAGCACCACT
ACGTGATCCCTTGGACCTTTGGCGGAGGCACCAAGCTGGAAATCAAGGGCAGCACA
AGCGGCTCTGGAAAACCTGGATCTGGCGAGGGCTCTACCAAGGGCGAGGTGCAGAT
GGTTGAGTCTGGCGGCGATCTGGTTAAGCCTGGCGGAAGCCTGAAGCTGTCTTGTG
CCGCCAGCGGCTTCACCTTCAGCAATAGCGGCATGAGCTGGGTCCGACAGACCCCT
GACAAGAGACTGGAATGGGTCGCCAGCATCTCTGACGGCGGCCTGTACACACACTA
CCCCGATTCTGTGAAGGGCAGATTCACCATCAGCAGAGACAACGGCAAGAGCACCC
TGTACCTGCAGATGAGCAGCCTGAGAAGCGAGGACACCGCCATCTACTACTGCGCC
AGACAGGGCGTCAGACCCTTCTTCGATTATTGGGGCCAGGGCACCACACTGACCGT
GTCATCTGGGAAGCCTATCCCGAACCCTCTGTTGGGTCTCGATAGTACCAATGGGGC
CGCAACCACGACGCCAGCGCCGCGACCACCAACACCGGCGCCCACCATCGCGTTGC
AGCCCCTGTCCCTGCGCCCAGAGGCGTGCCGGCCAGCGGCGGGGGGCGCAGTGCAC
ACGAGGGGGCTGGACTTCGCCTGTGATATCGTGGTGGGCGTGGTGTGCACCCTGCT
GGTGGCCCTGCTGATGGCCGCCCTGTACCTGGTGAGAATCAGACAGAAGAAGGCCC
AGGGCAGCACCAGCAGCACCAGACTGCACGAGCCCGAGAAGAACGCCAGAGAGAT
CACCCAGGACACCAACGACATCACCTACGCCGACCTGAACCTGCCCAAGGGCAAGA
AGCCCGCCCCCCAGGCCGCCGAGCCCAACAACCACACCGAGTACGCCAGCATCCAG
ACCAGCCCCCAGCCCGCCAGCGAGGACACCCTGACCTACGCCGACCTGGACATGGT
GCACCTGAACAGAACCCCCAAGCAGCCCGCCCCCAAGCCCGAGCCCAGCTTCAGCG
AGTACGCCAGCGTGCAGGTGCCCAGAAAG.

In some embodiments, the iCAR that binds VSIG2 that comprises the amino acid sequence of MALPVTALLLPLALLLHAARPDIQMTQSPASLSASVGETVTMTCRASENIYSYLAWYQ QKQGKSPQLLVFNAETLPEGVPSRFSGTGSGTHFSLRINSLQPEDFGSYYCQHHYVIPWT FGGGTKLEIKGSTSGSGKPGSGEGSTKGEVQMVESGGDLVKPGGSLKLSCAASGFTFSN SGMSWVRQTPDKRLEWVASISDGGLYTHYPDSVKGRFTISRDNGKSTLYLQMSSLRSE DTAIYYCARQGVRPFFDYWGQGTTLTVSSNGAATTTPAPRPPTPAPTIALQPLSLRPEAC RPAAGGAVHTRGLDFACDIVVGVVCTLLVALLMAALYLVRIRQKKAQGSTSSTRLHEP EKNAREITQDTNDITYADLNLPKGKKPAPQAAEPNNHTEYASIQTSPQPASEDTLTYAD LDMVHLNRTPKQPAPKPEPSFSEYASVQVPRK (SEQ ID NO: 421). An exemplary nucleic acid sequence encoding SEQ ID NO: 421 is

(SEQ ID NO: 422)
ATGGCCTTACCAGTGACCGCCTTGCTCCTGCCGCTGGCCTTGCTGCTCCACGCCGCC
AGGCCGGACATCCAGATGACACAGTCTCCAGCCAGCCTGTCTGCCTCTGTGGGAGA
GACAGTGACCATGACCTGTCGGGCCAGCGAGAACATCTACAGCTACCTGGCCTGGT
ATCAGCAGAAGCAGGGCAAGTCTCCTCAGCTGCTGGTGTTCAACGCCGAGACACTG
CCTGAAGGCGTGCCCAGCAGATTTTCTGGAACAGGCAGCGGCACCCACTTCAGCCT
GAGAATCAATAGCCTGCAGCCTGAGGACTTCGGCAGCTACTACTGCCAGCACCACT
ACGTGATCCCTTGGACCTTTGGCGGAGGCACCAAGCTGGAAATCAAGGGCAGCACA
AGCGGCTCTGGAAAACCTGGATCTGGCGAGGGCTCTACCAAGGGCGAGGTGCAGAT
GGTTGAGTCTGGCGGCGATCTGGTTAAGCCTGGCGGAAGCCTGAAGCTGTCTTGTG
CCGCCAGCGGCTTCACCTTCAGCAATAGCGGCATGAGCTGGGTCCGACAGACCCCT
GACAAGAGACTGGAATGGGTCGCCAGCATCTCTGACGGCGGCCTGTACACACACTA
CCCCGATTCTGTGAAGGGCAGATTCACCATCAGCAGAGACAACGGCAAGAGCACCC
TGTACCTGCAGATGAGCAGCCTGAGAAGCGAGGACACCGCCATCTACTACTGCGCC
AGACAGGGCGTCAGACCCTTCTTCGATTATTGGGGCCAGGGCACCACACTGACCGT
GTCATCTAATGGGGCCGCAACCACGACGCCAGCGCCGCGACCACCAACACCGGCGC
CCACCATCGCGTTGCAGCCCCTGTCCCTGCGCCCAGAGGCGTGCCGGCCAGCGGCG
GGGGGCGCAGTGCACACGAGGGGGCTGGACTTCGCCTGTGATATCGTGGTGGGCGT
GGTGTGCACCCTGCTGGTGGCCCTGCTGATGGCCGCCCTGTACCTGGTGAGAATCAG
ACAGAAGAAGGCCCAGGGCAGCACCAGCAGCACCAGACTGCACGAGCCCGAGAAG
AACGCCAGAGAGATCACCCAGGACACCAACGACATCACCTACGCCGACCTGAACCT
GCCCAAGGGCAAGAAGCCCGCCCCCCAGGCCGCCGAGCCCAACAACCACACCGAG
TACGCCAGCATCCAGACCAGCCCCCAGCCCGCCAGCGAGGACACCCTGACCTACGC
CGACCTGGACATGGTGCACCTGAACAGAACCCCCAAGCAGCCCGCCCCCAAGCCCG
AGCCCAGCTTCAGCGAGTACGCCAGCGTGCAGGTGCCCAGAAAG.

In cases wherein the immunoresponsive cells comprise an ACP, the ACP of the immunoresponsive cells described herein includes a synthetic transcription factor. A synthetic transcription factor is a non-naturally occurring protein that includes a DNA-binding domain and a transcriptional effector domain and is capable of modulating (i.e., activating or repressing) transcription through binding to a cognate promoter recognized by the DNA-binding domain. In some embodiments, the ACP is a transcriptional repressor. In some embodiments, the ACP is a transcriptional activator.

### Engineered Cell Types

Also provided herein are engineered immunoresponsive cells. Immunoresponsive cells can be engineered to comprise any of the engineered nucleic acids described herein (e.g., any of the engineered nucleic acids encoding the cytokines, membrane-cleavable chimeric proteins, and/or CARs described herein). Cells can be engineered to possess any of the features of any of the engineered cells described herein. In a particular aspect, provided herein are cells engineered to produce two cytokines and a CAR, where at least one of the cytokines is membrane-cleavable chimeric protein having the formula S-C-MT or MT-C-S described herein. Also provided herein are cells engineered to produce two cytokines, an aCAR, and an iCAR, where at least one of the cytokines is membrane-cleavable chimeric protein having the formula S-C-MT or MT-C-S described herein.

The engineered immunoresponsive cells include, but are not limited to, a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell. In particular embodiments, the immunoresponsive cell is a NK cell or a T cell. In some embodiments, the immunoresponsive cell is an NK cell.

A cell can be engineered to produce the proteins described herein using methods known to those skilled in the art. For example, cells can be transduced to engineer the tumor. In an embodiment, the cell is transduced using a virus.

In a particular embodiment, the cell is transduced using an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof.

The virus, including any of the oncolytic viruses described herein, can be a recombinant virus that encodes one more transgenes encoding one or more proteins, such as any of the engineered nucleic acids described herein. The virus, including any of the oncolytic viruses described herein, can be a recombinant virus that encodes one more transgenes encoding one or more of the two or more proteins, such as any of the engineered nucleic acids described herein.

Also provided herein are engineered bacterial cells. Bacterial cells can be engineered to comprise any of the engineered nucleic acids described herein. Bacterial cells can be engineered to possess any of the features of any of the engineered cells described herein. In a particular aspect, provided herein are bacterial cells engineered to produce two or more of the proteins described herein. Bacterial cells can be engineered to produce one or more mammalian-derived proteins. Bacterial cells can be engineered to produce two or more mammalian-derived proteins. Examples of bacterial cells include, but are not limited to, Clostridium beijerinckii, Clostridium sporogenes, Clostridium novyi, Escherichia coli, Pseudomonas aeruginosa, Listeria monocytogenes, Salmonella typhimurium, and Salmonella choleraesuis.

An engineered cell can be a human cell. An engineered cell can be a human primary cell. An engineered primary cell can be a tumor infiltrating primary cell. An engineered primary cell can be a primary T cell. An engineered primary cell can be a hematopoietic stem cell (HSC). An engineered primary cell can be a natural killer (NK) cell. An engineered primary cell can be any somatic cell. An engineered primary cell can be a MSC. Human cells (e.g., immune cells) can be engineered to comprise any of the engineered nucleic acids described herein. Human cells (e.g., immune cells) can be engineered to possess any of the features of any of the engineered cells described herein. In a particular aspect, provided herein are human cells (e.g., immune cells) engineered to produce one or more of the proteins described herein. In a particular aspect, provided herein are human cells (e.g., immune cells) engineered to produce two or more of the proteins described herein.

An engineered cell can be isolated from a subject (autologous), such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof.

An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

### Methods of Engineering Cells

Also provided herein are compositions and methods for engineering immunoresponsive cells to produce one or more proteins of interest (e.g., the cytokines, CARs, ACPs, and/or membrane-cleavable chimeric proteins having the formula S-C-MT or MT-C-S described herein).

In general, cells are engineered to produce proteins of interest through introduction (i.e., delivery) of polynucleotides encoding the one or more proteins of interest or effector molecules, e.g., the chimeric proteins described herein including the protein of interest or effector molecule, into the cell's cytosol and/or nucleus. For example, the polynucleotides encoding the one or more chimeric proteins can be any of the engineered nucleic acids encoding the cytokines, CARs, or membrane-cleavable chimeric proteins having the formula S-C-MT or MT-C-S described herein. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

### Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein (e.g., any of the exogenous polynucleotide sequences encoding the cytokines, CARs, ACPs, and/or the membrane-cleavable chimeric proteins having the formula S-C-MT or MT-C-S described herein, and/or any of the expression cassettes described herein containing a promoter and an exogenous polynucleotide sequence encoding the proteins, oriented from N-terminal to C-terminal). A viral vector-based delivery platform can be a nucleic acid, and as such, an engineered nucleic acid can also encompass an engineered virally-derived nucleic acid. Such engineered virally-derived nucleic acids can also be referred to as recombinant viruses or engineered viruses.

A viral vector-based delivery platform can encode more than one engineered nucleic acid, gene, or transgene within the same nucleic acid. For example, an engineered virally-derived nucleic acid, e.g., a recombinant virus or an engineered virus, can encode one or more transgenes, including, but not limited to, any of the engineered nucleic acids described herein that encode one or more of the proteins described herein. The one or more transgenes encoding the one or more proteins can be configured to express the one or more proteins and/or other protein of interest. A viral vector-based delivery platform can encode one or more genes in addition to the one or more transgenes (e.g., transgenes encoding the one or more proteins and/or other protein of interest), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the one or more proteins and/or other protein of interest. One viral vector can deliver more than one engineered nucleic acids, such as one vector that delivers engineered nucleic acids that are configured to produce two or more proteins and/or other protein of interest. More than one viral vector can deliver more than one engineered nucleic acids, such as more than one vector that delivers one or more engineered nucleic acid configured to produce one or more proteins and/or other protein of interest. The number of viral vectors used can depend on the packaging capacity of the above mentioned viral vector-based vaccine platforms, and one skilled in the art can select the appropriate number of viral vectors.

In general, any of the viral vector-based systems can be used for the in vitro production of molecules, such as the proteins, effector molecules, and/or other protein of interest described herein, or used in vivo and ex vivo gene therapy procedures, e.g., for in vivo delivery of the engineered nucleic acids encoding one or more proteins and/or other protein of interest. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, a adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuman et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e. delivery) into a host cell, infected cells (i.e., an engineered cell) can express the proteins and/or other protein of interest. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding one or more proteins and/or other protein of interest. The transgenes encoding the one or more proteins and/or other protein of interest can be configured to express the proteins and/or other protein of interest.

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding the one or more proteins and/or other protein of interest) into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immuno deficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of proteins of interest, such as the proteins described herein and/or effector molecules, or used in vivo and ex vivo gene therapy procedures, e.g., for in vivo delivery of the engineered nucleic acids encoding one or more proteins and/or other protein of interest (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat & amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof. In particular examples, an AAV-based vector has a capsid protein having an amino acid sequence corresponding to AAV2. In particular examples, an AAV-based vector has a capsid protein having an amino acid sequence corresponding to AAV8.

AAV vectors can be engineered to have any of the exogenous polynucleotide sequences encoding the proteins described herein, such as the cytokines, CARs, ACPs, and/or membrane-cleavable chimeric proteins described herein having the formula: S-C-MT or MT-C-S.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

### Lipid Structure Delivery Systems

Engineered nucleic acids (e.g., any of the engineered nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szokan et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl- 4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

### Nanoparticle Delivery

Nanomaterials can be used to deliver engineered nucleic acids (e.g., any of the engineered nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery-A Review. Nanomaterials 2017, 7(5), 94), herein incorporated by reference for all purposes.

### Genomic Editing Systems

A genomic editing systems can be used to engineer a host genome to encode an engineered nucleic acid, such as an engineered nucleic acid encoding the cytokines, CARs, ACPs, and/or membrane-cleavable chimeric proteins having the formula S-C-MT or MT-C-S described herein. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as the cytokines, CARs, ACPs, and/or membrane-cleavable chimeric proteins having the formula S-C-MT or MT-C-S described herein, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an engineered nucleic acid encoding the cytokines, CARs, ACPs, and/or the membrane-cleavable chimeric proteins having the formula S-C-MT or MT-C-S described herein. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding the cytokines, CARs, ACPs, and/or membrane-cleavable chimeric proteins having the formula S-C-MT or MT-C-S described herein).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding the cytokines, CARs, ACPs, and/or membrane-cleavable chimeric proteins having the formula S-C-MT or MT-C-S described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and a RNA(s) that directs cleavage to a particular target sequence. An exemplary CRISPR-mediated gene editing system is the CRISPR/Cas9 systems comprised of a Cas9 nuclease and a RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1/Cas12 or Cas13 systems. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA- binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### Other Engineering Delivery Systems

Various additional means to introduce engineered nucleic acids (e.g., any of the engineered nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., any of the engineered nucleic acids described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60.), each herein incorporated by reference for all purposes.

### Delivery Vehicles

Also provided herein are compositions for delivering a cargo/payload (a “delivery vehicle”).

The cargo can comprise nucleic acids (e.g., any of the engineered nucleic acids described herein, such as any of the engineered nucleic acids described herein encoding the cytokines, CARs, ACPs, and/or membrane-cleavable chimeric proteins having the formula S-C-MT or MT-C-S described herein), as described above. The cargo can comprise proteins, carbohydrates, lipids, small molecules, and/or combinations thereof.

The delivery vehicle can comprise any composition suitable for delivering a cargo. The delivery vehicle can comprise any composition suitable for delivering a protein (e.g., any of the proteins described herein). The delivery vehicle can be any of the lipid structure delivery systems described herein. For example, a delivery vehicle can be a lipid-based structure including, but not limited to, a lipid-based nanoparticle, a liposome, a micelle, an exosome, a vesicle, an extracellular vesicle, a cell, or a tissue. The delivery vehicle can be any of the nanoparticles described herein, such as nanoparticles comprising lipids (as previously described), inorganic nanomaterials, and other polymeric materials.

The delivery vehicle can be capable of delivering the cargo to a cell, such as delivering any of the proteins described herein to a cell. The delivery vehicle can be capable of delivering the cargo to a cell, such as delivering any of the proteins described herein to a cell. The delivery vehicle can be configured to target a specific cell, such as configured with a re-directing antibody to target a specific cell. The delivery vehicle can be capable of delivering the cargo to a cell in vivo.

The delivery vehicle can be capable of delivering the cargo to a tissue or tissue environment (e.g., a tumor microenvironment), such as delivering any of the proteins described herein to a tissue or tissue environment in vivo. Delivering a cargo can include secreting the cargo, such as secreting any of the proteins described herein. Accordingly, the delivery vehicle can be capable of secreting the cargo, such as secreting any of the proteins described herein. The delivery vehicle can be capable of secreting the cargo to a tissue or tissue environment (e.g., a tumor microenvironment), such as secreting any of the proteins described herein into a tissue or tissue environment. The delivery vehicle can be configured to target a specific tissue or tissue environment (e.g., a tumor microenvironment), such as configured with a re-directing antibody to target a specific tissue or tissue environment.

### Methods of Treatment

Further provided herein are methods that include delivering, or administering, to a subject (e.g., a human subject) engineered cells as provided herein to produce in vivo at least one protein of interest produced by the engineered cells (e.g., any of the cytokines, CARs, ACPs, and/or membrane-cleavable chimeric proteins having the formula S-C-MT or MT-C-S described herein, or the secreted effector molecules provided for herein following protease cleavage of the chimeric protein). Further provided herein are methods that include delivering, or administering, to a subject (e.g., a human subject) engineered cells as provided herein to produce in vivo at least two proteins of interest, e.g., at least two of the cytokines, CARs, ACPs, and/or membrane-cleavable chimeric proteins having the formula S-C-MT or MT-C-S described herein, produced by the engineered cells.

Further provided herein are methods that include delivering, or administering, to a subject (e.g., a human subject) any of the delivery vehicles described herein, such as any of the delivery vehicles described herein comprising any of the proteins of interest described herein, e.g., any of the cytokines, CARs, ACPs, and/or membrane-cleavable chimeric proteins having the formula S-C-MT or MT-C-S described herein. Further provided herein are methods that include delivering, or administering, to a subject (e.g., a human subject) any of the delivery vehicles described herein, such as any of the delivery vehicles described herein comprising two or more proteins of, e.g., at least two of the cytokines, CARs, ACPs, and/or the membrane-cleavable chimeric proteins having the formula S-C-MT or MT-C-S described herein.

In some embodiments, the engineered cells or delivery vehicles are administered via intravenous, intraperitoneal, intratracheal, subcutaneous, intratumoral, oral, anal, intranasal (e.g., packed in a delivery particle), or arterial (e.g., internal carotid artery) routes. Thus, the engineered cells or delivery vehicles may be administered systemically or locally (e.g., to a TME or via intratumoral administration). An engineered cell can be isolated from a subject, such as a subject known or suspected to have cancer. An engineered cell can be allogenic with reference to the subject being administered a treatment. Allogenic modified cells can be HLA-matched to the subject being administered a treatment. Delivery vehicles can be any of the lipid structure delivery systems described herein. Delivery vehicles can be any of the nanoparticles described herein.

Engineered cells or delivery vehicles can be administered alone or in combination with other treatments, either simultaneously or sequentially dependent upon the condition to be treated. For example, engineered cells or delivery vehicles can be administered in combination with one or more IMiDs described herein. FDA-approved IMiDs can be administered in their approved fashion. In another example, engineered cells or delivery vehicles can be administered in combination with a checkpoint inhibitor therapy. Exemplary checkpoint inhibitors include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies. Illustrative immune checkpoint inhibitors include pembrolizumab (anti-PD-1; MK-3475/Keytruda®—Merck), nivolumamb (anti-PD-1; Opdivo®—BMS), pidilizumab (anti-PD-1 antibody; CT-011—Teva/CureTech), AMP224 (anti-PD-1; NCI), avelumab (anti-PD-L1; Bavencio®—Pfizer), durvalumab (anti-PD-L1; MED14736/Imfinzi®—Medimmune/AstraZeneca), atezolizumab (anti-PD-L1; Tecentriq®-Roche/Genentech), BMS-936559 (anti-PD-L1—BMS), tremelimumab (anti-CTLA-4; Medimmune/AstraZeneca), ipilimumab (anti-CTLA-4; Yervoy®—BMS), lirilumab (anti-KIR; BMS), monalizumab (anti-NKG2A; Innate Pharma/AstraZeneca). In other examples, engineered cells or delivery vehicles can be administered in combination with TGFbeta inhibitors, VEGF inhibitors, or HPGE2. In another example, engineered cells or delivery vehicles can be administered in combination with an anti-CD40 antibody.

Some methods comprise selecting a subject (or patient population) having a tumor (or cancer) and treating that subject with engineered cells or delivery vehicles that modulate tumor-mediated immunosuppressive mechanisms.

The engineered cells or delivery vehicles of the present disclosure may be used, in some instances, to treat cancer, such as ovarian cancer. Other cancers are described herein. For example, the engineered cells may be used to treat bladder tumors, brain tumors, breast tumors, cervical tumors, colorectal tumors, esophageal tumors, gliomas, kidney tumors, liver tumors, lung tumors, melanomas, ovarian tumors, pancreatic tumors, prostate tumors, skin tumors, thyroid tumors, and/or uterine tumors. The engineered cells or delivery vehicles of the present disclosure can be used to treat cancers with tumors located in the peritoneal space of a subject.

The methods provided herein also include delivering a preparation of engineered cells or delivery vehicles. A preparation, in some embodiments, is a substantially pure preparation, containing, for example, less than 5% (e.g., less than 4%, 3%, 2%, or 1%) of cells other than engineered cells. A preparation may comprise 1×10⁵ cells/kg to 1×10⁷ cells/kg cells. Preparation of engineered cells or delivery vehicles can include pharmaceutical compositions having one or more pharmaceutically acceptable carriers. For example, preparations of engineered cells or delivery vehicles can include any of the engineered viruses, such as an engineered AAV virus, or any of the engineered viral vectors, such as AAV vector, described herein.

### In Vivo Expression

The methods provided herein also include delivering a composition in vivo capable of producing the engineered cells described herein, e.g., capable of delivering any of the engineered nucleic acids described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the proteins of interest described herein, e.g., any of the cytokines, CARs, ACPs, and/or membrane-cleavable chimeric proteins having the formula S-C-MT or MT-C-S described herein. The methods provided herein also include delivering a composition in vivo capable of producing two or more of the proteins of interest described herein. Compositions capable of in vivo production of proteins of interest include, but are not limited to, any of the engineered nucleic acids described herein. Compositions capable of in vivo production proteins of interest can be a naked mRNA or a naked plasmid.

### Additional Embodiments

1. A multicistronic expression system comprising:
   (a) an exogenous polynucleotide sequence encoding a first cytokine;
   (b) an exogenous polynucleotide sequence encoding a second cytokine; and
   (c) an exogenous polynucleotide sequence encoding a chimeric antigen receptor (CAR),
   wherein each exogenous polynucleotide sequence comprises a 5′ end and a 3′ end.
2. The multicistronic expression system of Embodiment 1, wherein at least one of the first and the second cytokines is a controlled release cytokine.
3. The multicistronic expression system of Embodiment 2, wherein each controlled release cytokine has the formula:

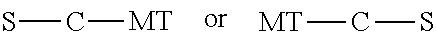

wherein
   S comprises a secretable effector molecule;
   C comprises a protease cleavage site; and
   MT comprises a cell membrane tethering domain.
4. The multicistronic expression system of any one of Embodiments 1-3, wherein the protease cleavage site is cleaved by ADAM10 and/or ADAM17.
5. The multicistronic expression system of any one of Embodiments 1-4, wherein the protease cleavage site comprises the amino acid sequence set forth in SEQ ID NO: 180 or SEQ ID NO: 191.
6. The multicistronic expression system of any one of Embodiments 1-5, wherein the cell membrane tethering domain comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, LIR1, B7-1, and BTLA.
7. The multicistronic expression system of any one of Embodiments 1-6, wherein the cell membrane tethering domain comprises a B7-1 transmembrane domain comprising the amino acid sequence set forth in SEQ ID NO: 219.
8. The multicistronic expression system of any one of Embodiments 1-7, wherein the first cytokine is IL15.
9. The multicistronic expression system of Embodiment 8, wherein the IL15 comprises the amino acid sequence set forth in SEQ ID NO: 285.
10. The multicistronic expression system of Embodiment 8 or 9, wherein the IL15 is controlled-release IL15 (crIL15).
11. The multicistronic expression system of any one of Embodiments 1-10, wherein the second cytokine is IL21.
12. The multicistronic expression system of Embodiment 11, wherein the IL21 comprises the amino acid sequence set forth in SEQ ID NO: 360.
13. The multicistronic expression system of Embodiment 11 or 12, wherein the IL21 is controlled-release IL21 (crIL21).
14. The multicistronic expression system of Embodiment 1, wherein the first or second cytokine comprises an amino acid sequence set forth in any one of SEQ ID NOs: 355-359, 361, and 391.
15. The multicistronic expression system of Embodiment 1 or 14, wherein the first or second cytokine is encoded by a nucleic acid sequence set forth in any one of SEQ ID NOs: -367-372, and 392.
16. The multicistronic expression system of any one of Embodiments 1-15, comprising an exogenous polynucleotide sequence encoding an activating CAR (aCAR) and an exogenous polynucleotide sequence encoding an inhibitory CAR (iCAR).
17. The multicistronic expression system of Embodiment 16, wherein the aCAR comprises:
   (a) a first antigen-binding domain;
   (b) one or more intracellular signaling domains that stimulate an immune response; and
   (c) one or more polypeptides selected from the group consisting of: a signal peptide, a transmembrane domain, a hinge domain, a spacer region, one or more peptide linkers, and combinations thereof.
18. The multicistronic expression system of Embodiment 17, wherein the first antigen-binding domain of the aCAR binds CEA, CEACAM1, CEACAM5, and CEACAM6.
19. The multicistronic expression system of Embodiment 17, wherein the first antigen-binding domain of the aCAR binds CEACAM5.
20. The multicistronic expression system of any one of Embodiments 17-19, wherein the first antigen binding domain of the aCAR comprises the amino acid sequence set forth in SEQ ID NO: 381.
21. The multicistronic expression system of Embodiment 17 or 18, wherein the one or more intracellular signaling domains of the aCAR are selected from the group consisting of: CD3-zeta, FcR gamma, FcR beta, CD3 gamma, CD3 delta, CD3 epsilon, CD5, CD22, CD79a, CD79b, CD278, FcεRI, DAP10, DAP12, CD66d, CD97, CD2, ICOS, CD27, CD154, CD8, OX40, 4-1BB, CD28, ZAP40, CD30, GITR, HVEM, DAP10, DAP12, MyD88, 2B4, CD40, PD-1, LFA-1, CD7, LIGHT, NKG2C, B7-H3, an MHC class I molecule, a TNF receptor protein, an Immunoglobulin-like protein, a cytokine receptor, an integrin, a SLAM protein, an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLAl, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and combinations thereof.
22. The multicistronic expression system of any one of Embodiments 17-21, wherein the aCAR comprises a hinge domain selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof.
23. The multicistronic expression system of any one of Embodiments 17-22, wherein the aCAR comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, and BTLA.
24. The multicistronic expression system of any one of Embodiments 17-23, wherein the aCAR comprises a signal peptide selected from the group consisting of: IgE, IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, CXCL12, IL21, CD8, NKG2D, TNFR2, GMCSF, and GM-CSFRa.
25. The multicistronic expression system of any one of Embodiments 16-24, wherein the aCAR comprises an amino acid sequence set forth in any one of SEQ ID NOs: 362-365.
26. The multicistronic expression system of any one of Embodiments 16-25, wherein the aCAR is encoded by a nucleic acid sequence set forth in any one of SEQ ID NOs: 373-376.
27. The multicistronic expression system of any one of Embodiments 16-26, wherein the iCAR comprises:
   (a) a second antigen-binding domain;
   (b) one or more intracellular signaling domains that inhibit an immune response; and
   (c) one or more polypeptides selected from the group consisting of: a signal peptide, a transmembrane domain, a hinge domain, a spacer region, one or more peptide linkers, and combinations thereof.
28. The multicistronic expression system of Embodiment 27, wherein the second antigen-binding domain of the iCAR binds VSIG2.
29. The multicistronic expression system of Embodiment 27 or 28, wherein the iCAR comprises an LIR1 intracellular inhibitory domain.
30. The multicistronic expression system of Embodiment 29, wherein the intracellular inhibitory domain comprises the amino acid sequence set forth in SEQ ID NO: 387.
31. The multicistronic expression system of Embodiment 27 or 28, wherein the iCAR comprises an SIRPα intracellular inhibitory domain.
32. The multicistronic expression system of Embodiment 31, wherein the intracellular inhibitory domain comprises the amino acid sequence set forth in SEQ ID NO: 385.
33. The multicistronic expression system of any one of Embodiments 27-29, wherein the iCAR comprises a hinge domain selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof.
34. The multicistronic expression system of any one of Embodiments 27-33, wherein the iCAR comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, SIRPα, and BTLA.
35. The multicistronic expression system of any one of Embodiments 27-34, wherein the iCAR comprises a signal peptide selected from the group consisting of: IgE, IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, CXCL12, IL21, CD8, NKG2D, TNFR2, GMCSF, and GM-CSFRa.
36. The multicistronic expression system of Embodiment 16, wherein the iCAR comprises the amino acid sequence set forth in SEQ ID NO: 366.
37. The multicistronic expression system of Embodiments 16 or 36, wherein the iCAR is encoded by the nucleic acid sequence set forth in SEQ ID NO: 377.
38. The multicistronic expression system of any one of Embodiments 16-37, wherein the exogenous polynucleotide encoding the first cytokine, the exogenous polynucleotide encoding the second cytokine, the exogenous polynucleotide encoding the aCAR, and the exogenous polynucleotide encoding the iCAR are comprised within a single expression vector.
39. The multicistronic expression system of any one of Embodiments 16-37, wherein the exogenous polynucleotide encoding the first cytokine, the exogenous polynucleotide encoding the second cytokine, and the exogenous polynucleotide encoding the aCAR are comprised within a first expression vector, and the exogenous polynucleotide encoding the iCAR is comprised within a second expression vector.
40. The multicistronic expression system of any one of Embodiments 1-39, wherein each exogenous polynucleotide sequence further comprises a promoter sequence at the 5′ end.
41. The multicistronic expression system of Embodiment 40, wherein the promoter is a constitutive promoter or an inducible promoter.
42. The multicistronic expression system of any one of Embodiments 1-41, further comprising ribosome skipping sites between each exogenous polynucleotide.
43. An engineered cell comprising the multicistronic expression system of any one of Embodiments 1-42.
44. The engineered cell of Embodiment 43, wherein the engineered cell is an immune cell.
45. The engineered cell of Embodiment 43, wherein the engineered cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell.
46. The engineered cell of any one of Embodiments 43-45, wherein the engineered cell is an NK cell.
47. A pharmaceutical composition comprising the engineered cell of any one of Embodiments 43-46 and a pharmaceutically acceptable carrier.
48. A method of treating a disease in a subjected in needed thereof, the method comprising administering a therapeutically effective dose of the engineered cell of any one of Embodiments 43-46 or the pharmaceutical composition of Embodiment 47 to the subject.
49. The method of Embodiment 48, wherein the disease is a cancer.
50. The method of Embodiment 48 or 49, wherein the isolated cell is allogenic to the subject.
51. The method of Embodiment 48 or 49, wherein the isolated cell is autologous to the subject.
52. A method of manufacturing an engineered cell, the method comprising transducing an isolated cell with the multicistronic expression system of any one of Embodiments 1-39.
53. The method of Embodiment 52, wherein the isolated cell is an immune cell.
54. The method of Embodiment 52, wherein the isolated cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell.
55. The isolated cell of any one of Embodiments 52-54, wherein the isolated cell is an NK cell.
56. An immunoresponsive cell comprising:
   (a) an exogenous polynucleotide encoding a first cytokine;
   (b) an exogenous polynucleotide encoding a second cytokine;
      an exogenous polynucleotide encoding a chimeric antigen receptor (CAR).

### Examples

Below are examples of specific embodiments for carrying out the present invention. The examples are offered for illustrative purposes only, and are not intended to limit the scope of the present invention in any way. For example, the experiments described and performed below demonstrate the general utility of engineering cells to secrete payloads (e.g., effector molecules) and delivering those cells to induce an immunogenic response against tumors.

Efforts have been made to ensure accuracy with respect to numbers used (e.g., amounts, temperatures, etc.), but some experimental error and deviation should, of course, be allowed for.

### Example 1: Expression and Function of an Anti-GPC3 CAR+IL15 Bidirectional Construct

Protein expression, cellular activation, and killing activity of cells transduced with anti-GPC3 CAR+IL15 bidirectional constructs were assessed. A cartoon diagram of the bidirectional orientation of the constructs is shown in FIG. 1.

### Materials and Methods

Primary, donor-derived NK cells were transduced (50,000 to 100,000 cells/transduction) in a non-TC treated retronectin coated plate with lentivirus (at a multiplicity of infection, MOI, of 40) or retrovirus (SinVec, approximately 400 μl each) encoding constructs having a first expression cassette encoding an anti-GPC3 CAR and a second expression cassette encoding IL15, with the two expression cassettes in a head-to-head bidirectional orientation. Constructs varied in the intracellular domains of the CAR, having 4-1BB and CD3-zeta signaling domains (41BBz), CD28 and CD3-zeta signaling domains (CD28z), OX40 and CD3-zeta signaling domains (OX40z) or a KIR3DS1 signaling domain (KIR3DS1), and transductions using either a lentivirus or a retrovirus system were compared for each construct. As a control, transductions were also performed with retroviruses and lentiviruses encoding each of the same CARs, but without the IL15 expression cassette (“CAR-only). After transduction, NK cells were rested in the same plate for 3 days before transfer to a 24-well non-adherent cell-optimized plate. NK cells were expanded to a total of 5 ml with a first cytokine spike-in on day 7 following transduction and a second cytokine spike-in on day 15 (each spike-in included 500 IU/ml IL12 for the CAR+IL15 transductions and the CAR-only transductions, and 10 ng/ml IL15 for the CAR only constructs).

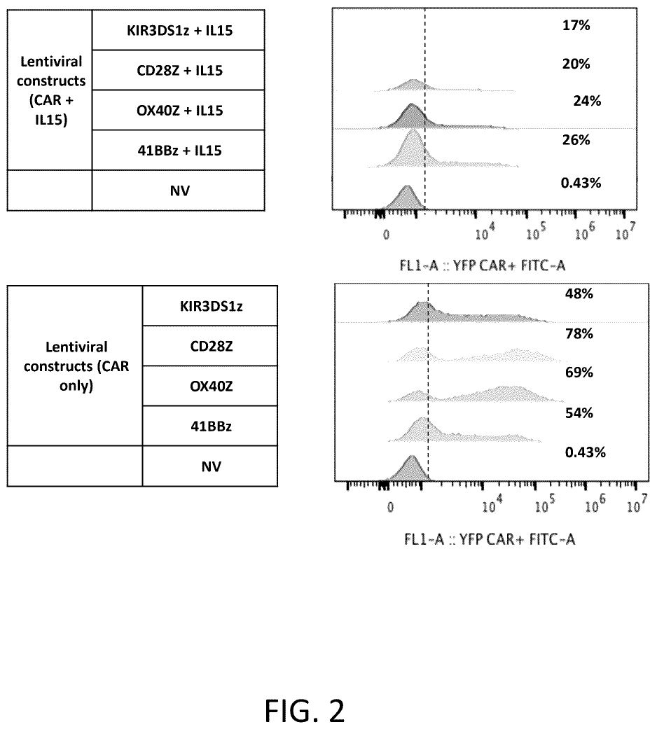

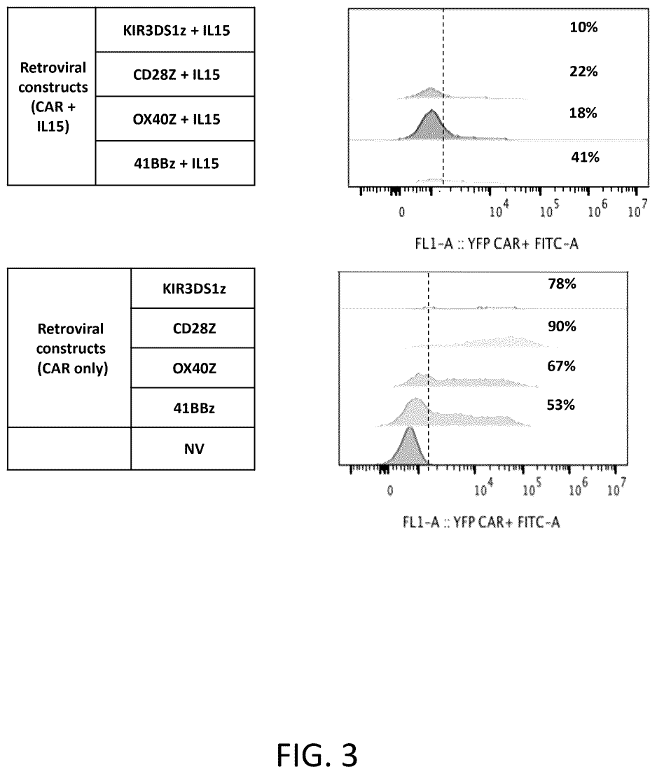

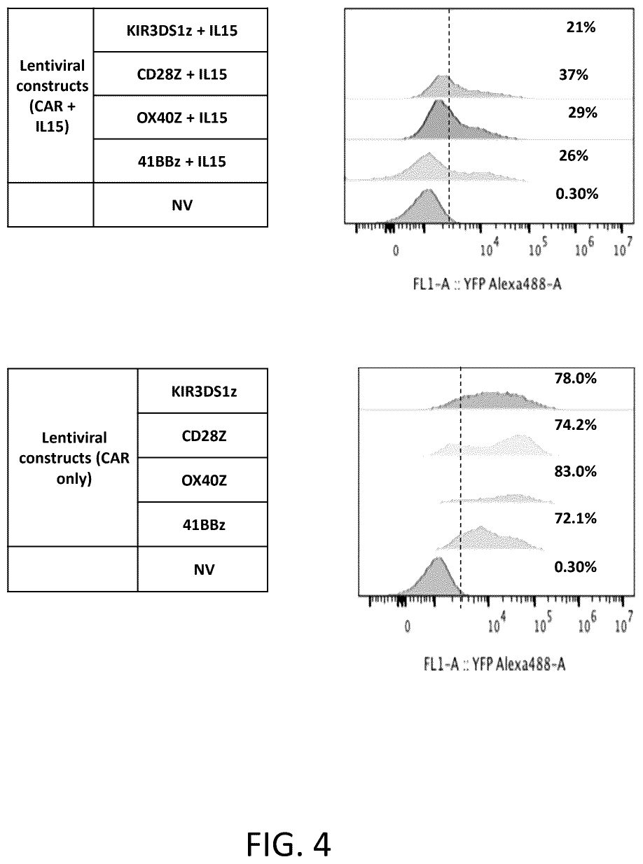

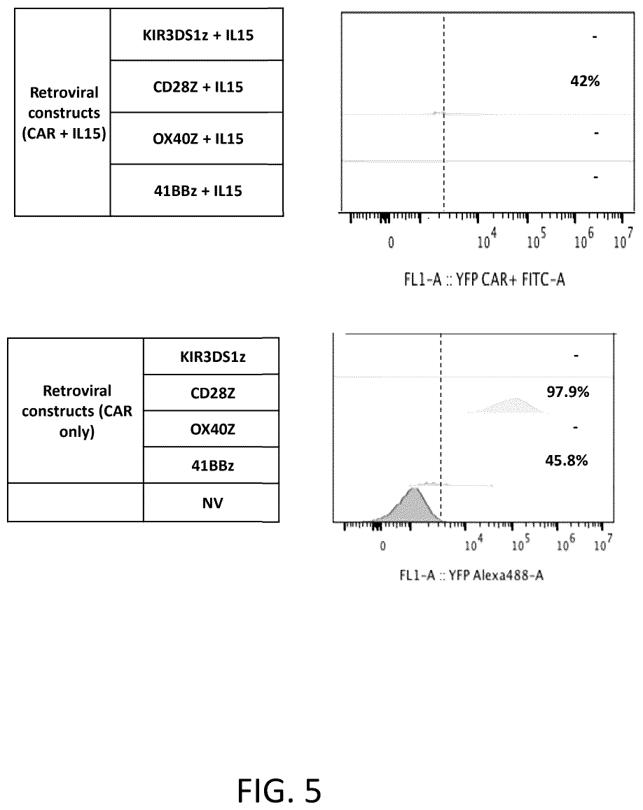

On days five and seven following transduction, CAR expression was assessed by flow cytometry for each construct. Day seven CAR expression from cells transduced with lentivirus encoding a bidirectional CAR+IL15 bidirectional construct and cells transduced with a lentivirus encoding the CAR-only is shown in FIG. 2. Day seven CAR expression from cells transduced with retrovirus encoding a bidirectional CAR+IL15 bidirectional construct and cells transduced with a retrovirus encoding the CAR-only is shown in FIG. 3. Day fifteen CAR expression from cells transduced with lentivirus encoding a bidirectional CAR+IL15 bidirectional construct and cells transduced with a lentivirus encoding the CAR-only is shown in FIG. 4. Day fifteen CAR expression from cells transduced with retrovirus encoding a bidirectional CAR+IL15 bidirectional construct and cells transduced with a retrovirus encoding the CAR-only is shown in FIG. 5.

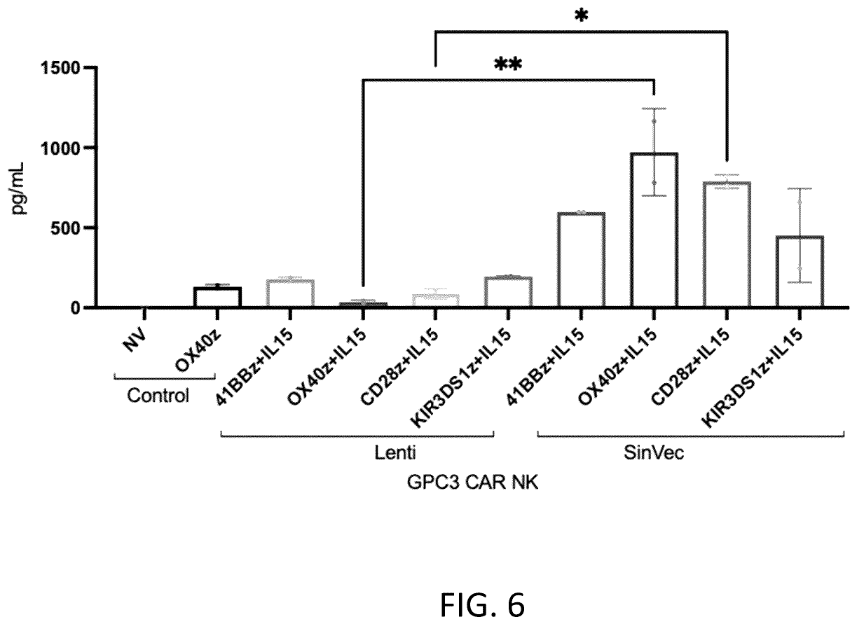

On day seven following transduction, a payload assay was conducted to assess IL15 levels for each construct. 200,000 cells per well were plated in 200 μl media (NK MACs complete media with IL2) in a 96-well plate. NK cells were incubated for 48 hours, and then IL15 levels were assessed by immunoassay. IL15 expression is shown in FIG. 6.

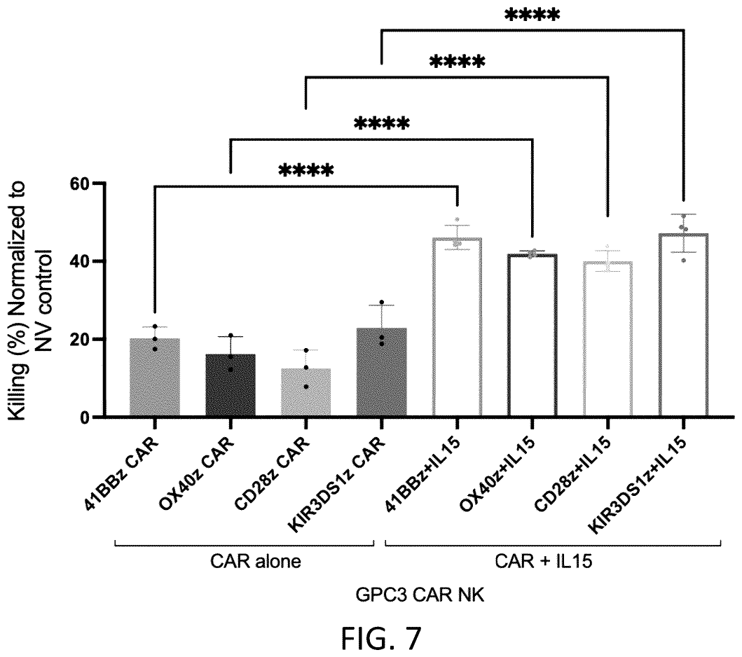

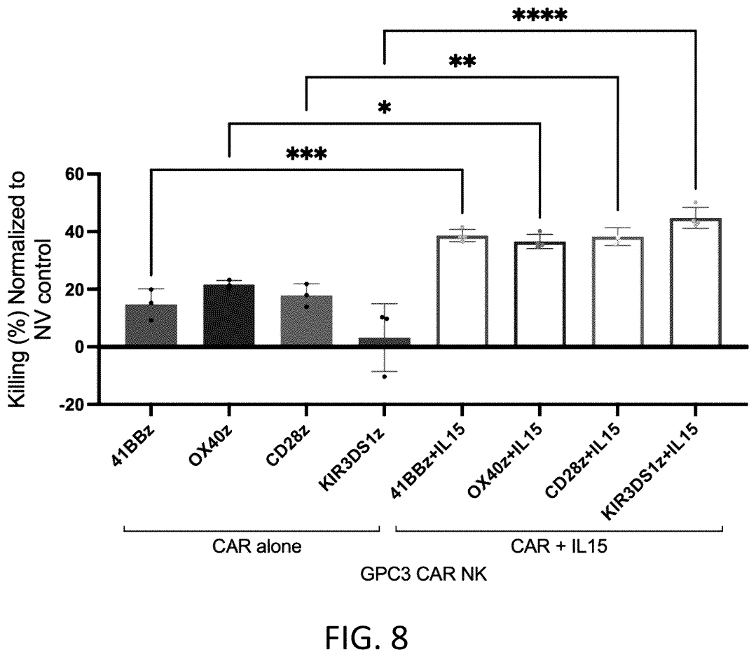

Co-culture killing assays were then performed. 25,000 target cells (a Huh7 mKate cell line or a HepG2 mKate cell line) per well were plated in a 96-well plate. Effector cells (the NK cells expressing each construct) were added to the plate at effector to target (E to T) cell ratios of 1:1 or 0.5:1, and the cells were cultured with NK MACs complete media without cytokines in a total volume of 200 μl. Two to three days following co-culture, real-time, fluorescence-based assays to measure mKate levels were performed to assess target cell killing. Killing by lentivirus-transduced NK cells expressing each construct is shown in FIG. 7, and killing by retrovirus-transduced NK cells expressing each construct is shown in FIG. 8.

### Results

CAR expression from NK cells transduced with each construct was assessed. As shown in FIG. 2, at day seven transduced NK cells had measurable CAR expression for each construct, with at least 10% of cells in each transduced population positive for CAR expression. As shown in FIG. 3, at day fifteen lentivirus-transduced NK cells had measurable CAR expression for each construct (top panel), with at least 20% of cells in each transduced population positive for CAR expression. Additionally, as shown in FIG. 3, retrovirus-transduced NK cells expressing the 28z CAR+IL15 bidirectional construct had measurable CAR expression, with at least 42% of cells in the transduced population positive for CAR expression.

IL15 expression by NK cells transduced with each construct was also assessed. Assay of IL15 expression by non-transduced cells and Ox40z CAR-only cells was performed as a negative control. As shown in FIG. 6, retrovirus-transduced NK cells expressing bidirectional CAR+IL15 had statistically significant increase in IL15 production over reciprocal lentivirus-transduced NK cells.

Killing by NK cells transduced with each construct was then assessed. As shown in FIG. 7, lentivirus-transduced NK cells expressing the CAR+IL15 bidirectional construct had statistically significant increased killing over lentivirus-transduced NK cells expressing the CAR alone (without the IL15 expression cassette). As shown in FIG. 8, retrovirus-transduced NK cells expressing the CAR+IL15 bidirectional construct had statistically significant increased killing over retrovirus-transduced NK cells expressing the CAR alone (without the IL15 expression cassette).

### Example 2: Expression of IL12 from Bidirectional Constructs Encoding a Regulatable IL12 and a Synthetic Transcription Factor

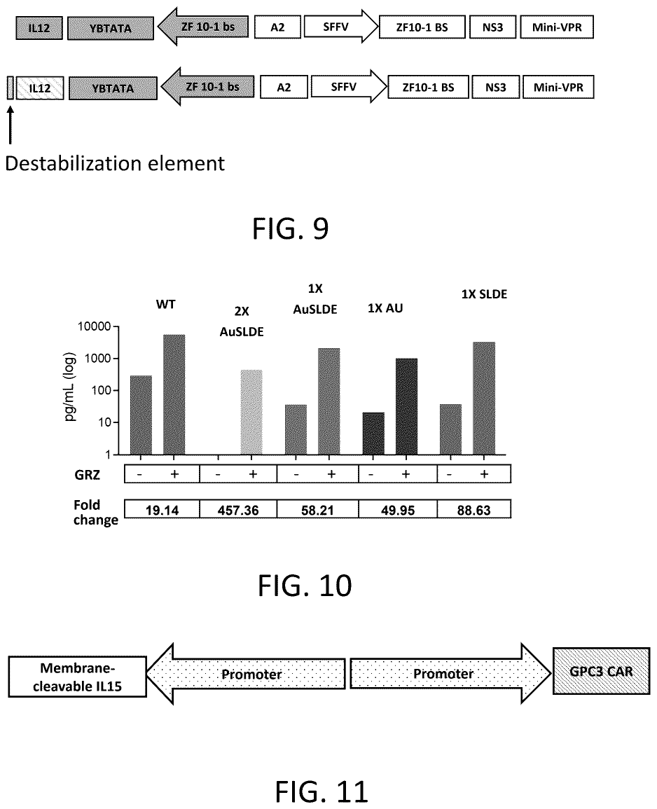

IL12 expression was assessed from NK cells transduced to express bidirectional constructs including a first expression cassette encoding a regulatable IL12 and a second expression cassette encoding a synthetic transcription factor. The regulatable IL12 is operably linked to a synthetic transcription factor-responsive promoter, which includes a ZF-10-1 binding site and a minimal promoter sequence (YBTATA)(SEQ ID NO:443). The synthetic transcription factor includes a DNA binding domain (an array of six zinc finger motifs known as ZF-10-1) and a transcriptional activation domain (Vpr). Between the DNA biding domain and the transcriptional activation domain is a protease domain (NS3) and cognate cleavage site for the protease. In the absence of an inhibitor of the protease, the protease induces cleavage at the cleavage site, resulting in a non-functional synthetic transcription factor. In the presence of the protease inhibitor, the synthetic transcription factor is not cleaved and is thus capable of modulating expression of the IL12. Constructs tested included IL12 expression cassettes having mRNA destabilization elements in the 3′ untranslated region. A cartoon diagram of the bidirectional orientation of the constructs is shown in FIG. 9(SEQ ID NO:443).

### Materials and Methods

Bidirectional constructs including two expression cassettes, a first expression cassette encoding a regulatable IL12 and a second expression cassette encoding a small molecule-regulatable synthetic transcription factor, were produced. A first construct lacks an mRNA destabilization element (“WT”), and four constructs each include a different mRNA destabilization element added to the 5′ non-coding region. The four destabilization elements used were: 1) an AU-rich motif (“AU” or “1XAU”); 2) a stem-loop destabilization element (“SLDE” or “1XSLDE”); 3) a tandem AU motif and SLDE motif (“AuSLDE” or “1× AuSLDE”); and 4) two repeated AuSLDE motifs (2× AuSLDE). The destabilization elements were added to attempt to reduce leakiness of IL12 expression in the absence of the small molecule regulator of the synthetic promoter (e.g., grazoprevir).

Primary, donor-derived NK cells were expanded for ten days and grown in IL21 and IL15, with K562 feeder cells, and then were transduced with a multiplicity of infection (MOI) of 40 (as determined by infection units titer) in a retronectin-coated 24 well plate, following Bx795 pre-treatment. Transduction was performed with spinoculation, at 800 g for 2 hours at 32° C.

On day three following transduction, NK cells were counted and seeded at 1e6 cells/mL with no drug or 0.1 uM grazoprevir (GRZ) for 24 hours.

On day four following transduction (with 24 hours of drug treatment), supernatant was harvested and analyzed for IL12 levels by immunoassay. IL12 concentrations for each cell type and condition are shown in FIG. 10.

### Results

As shown in FIG. 10, NK cells transduced with each construct demonstrated increased IL12 expression following treatment with grazoprevir, as compared to the absence of drug. NK cells transduced with the IL12 lacking a destabilization element (“WT”) had greater than 19-fold induction of IL12 expression following treatment with grazoprevir. However, NK cells transduced with constructs that included destabilization tags demonstrated about a 457-fold, 58-fold, 50-fold, and 89-fold induction of IL12 upon treatment with grazoprevir for 2× AuSLDE, 1× AuSLDE, 1× AU, and 1× SLDE, respectively. Additionally, each of the destabilization tags decreased the baseline IL12 expression in the absence of grazoprevir. Furthermore, the construct encoding an IL12 with a 2× AuSLDE destabilization element resulted in a non-detectable level of IL12 expression in the absence of grazoprevir.

### Example 3: Expression and Function of Anti-GPC3 CAR+IL15 Bidirectional Constructs

Protein expression, cellular activation, and killing activity of cells transduced with anti-GPC3 CAR+cleavable release IL15 bidirectional constructs were assessed. The expression cassette encoding the cleavable release IL15 includes a chimeric polypeptide including the IL15 and a transmembrane domain. Between the IL15 and the transmembrane domain is a protease cleavage domain that is cleavable by a protease endogenous to NK cells. A cartoon diagram of the bidirectional construct encoding a cleavable release IL15 is shown in FIG. 11.

Briefly, primary, donor-derived NK cells were transduced with viral vectors encoding constructs having a first expression cassette encoding an anti-GPC3 CAR and a second expression cassette encoding a cleavable release IL15 expression cassette, with the two expression cassettes in a head-to-head bidirectional orientation.

Culture Supernatant: Spinoculation of NK cells was performed (day 0). A partial culture media exchange was performed on days 1, 2, and 6. Cell culture supernatant was harvested on day 8.

Flow cytometry: On day 10 following transduction, CAR and mbIL15 expression was assessed by flow cytometry for each construct. NK cells were stained with an IL15 primary antibody and PE-secondary, and rhGPC3-FITC and Sytox blue (viability stain). Cells were run on Cytoflex and analyzed using Flowjo for CAR/mbIL15 expression.

Payload assay: On day 7 or 8 following transduction, a payload assay was conducted to assess IL15 levels for each construct. 200,000 cells per well were plated in 200 μl media (NK MACs complete media with IL2 only) in a 96-well plate, run in duplicates. Cells were incubated for 48 hours, and then cleaved IL15 levels were assessed by Luminex immunoassay.

Serial killing assay: Co-culture killing assays were performed. About 25,000 target cells (a Huh7 mKate cell line or a HepG2 mKate cell line) per well were plated in a 96-well plate. Effector cells (the NK cells expressing each construct) were added to the plate at effector to target (E to T) cell ratios of 1:1 in triplicates, and the cells were cultured with NK MAC complete media (no cytokines) in a total volume of 200 μl. Real-time, fluorescence-based assays were used to measure mKate to assess target cell killing in a serial-killing assay performed at 37° C.; initial killing was at day 9 post-transduction, serial one was at day 11 post-transduction, and serial 2 was at day 14 post transduction.

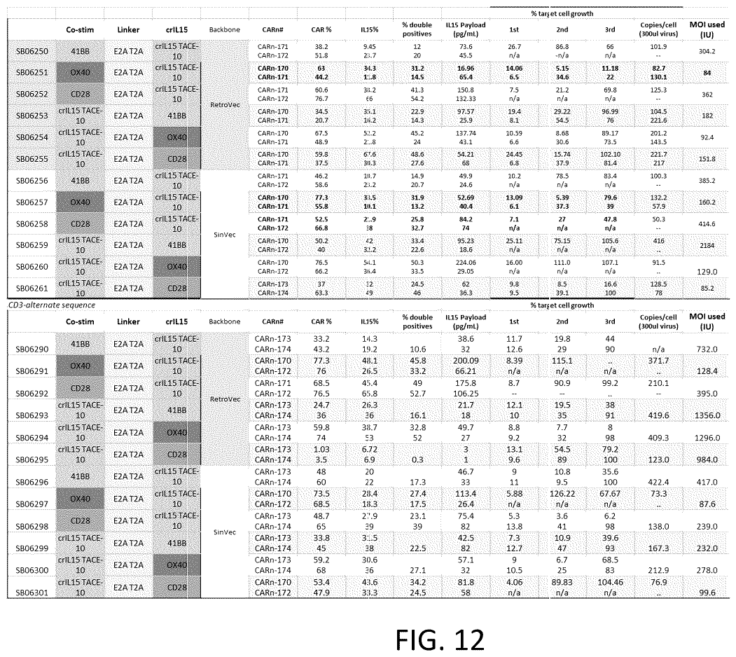

Over 150 IL15 cleavable release (crIL15) constructs were designed, and 33 constructs were selected for experimental testing. (see Table 7A). Each construct was tested in two viral backbones (e.g., SB06250 and SB06256, as shown in Table 7A). A summary of expression and killing activity of cells expressing a subset of bicistronic constructs is shown in Table 7B. Full-length sequences of a subset of constructs are shown in Table 7C. A summary of bicistronic constructs tested and their functional activities is provided in FIG. 12.

TABLE 7A

| Construct | SB# (CD3 Senti) | SB# (CD3mut) |
|---|---|---|
| GPC3-CAR (41BB) 2A crIL15(Tace10) | SB06250 | SB06290 |
| | SB06256 | SB06296 |
| GPC3-CAR (OX40) 2A crIL15(Tace10) | SB06251 | SB06291 |
| | SB06257 | SB06297 |
| GPC3-CAR (CD28) 2A crIL15(Tace10) | SB06252 | SB06292 |
| | SB06258 | SB06298 |
| crIL15(Tace10) 2A GPC3-CAR (41BB) | SB06253 | SB06293 |
| | SB06259 | SB06299 |
| crIL15(Tace10) 2A GPC3-CAR (OX40) | SB06254 | SB06294 |
| | SB06260 | SB06300 |
| crIL15(Tace10) 2A GPC3-CAR (CD28) | SB06255 | SB06295 |
| | SB06261 | SB06301 |
| crIL15(TaceOPT) 2A GPC3-CAR (41BB) | SB06685 | SB06688 |
| | SB06691 | SB06694 |
| crIL15(TaceOPT) 2A GPC3-CAR (OX40) | SB06686 | SB06689 |
| | SB06692 | SB06695 |
| crIL15(TaceOPT) 2A GPC3-CAR (CD28) | SB06687 | SB06690 |
| | SB06693 | SB06696 |

TABLE 7B

| | | | | | % Target | | | | | | |
|---|---|---|---|---|---|---|---|---|---|---|---|
| | | | | | cell | | | | | | |
| | | | | | growth | | | | | | |
| Virus | SB# | CAR % | IL15% | IL15(pg/ml) | (round3)^{a} | Hinge | TM | Co-stim | CD3z | IL15 | Description |
| Retrovec | SB06252 | 76.7 | 64.8 | 151 | n/a | CD8FA | CD8FA | CD28 | wt | Tace10 | CAR 2A crIL15 |
| | | 60.6 | 51.2 | 117 | 70 | | | | | | |
| SinVec | SB06258 | 66.8 | 38.5 | 84 | n/a | | | | | | |
| | | 52.5 | 30.6 | 74 | 62 | | | | | | |
| Retrovec | SB06255 | 59.8 | 67.6 | 54 | n/a | | | | | | crIL15 2A CAR |
| | | 37.5 | 41.0 | 58 | 81.4 | | | | | | |
| Retrovec | SB06251 | 64.2 | 30.9 | 17 | 11.2* | CD8 | OX40 | OX40 | wt | Tace10 | CAR 2A crIL15 |
| | | 44.2 | 18.5 | 65 | 22 | S2L | | | | | |
| SinVec | SB06257 | 78.3 | 30.1 | 53 | 59* | | | | | | |
| | | 55.8 | 15.8 | 40 | 39 | | | | | | |
| Retrovec | SB06254 | 67.5 | 52.2 | 137 | 89* | | | | | | crIL15 2A CAR |
| | | 48.9 | 30.1 | 43 | 74 | | | | | | |
| Retrovec | SB06294 | 60 | 39 | 50 | 8 | CD8 | OX40 | OX40 | CD3z- | Tace10 | crIL15 2A CAR |
| | | 71 | 58 | 27 | | S2L | | | Alt | | |

^{a}Normalized to Target cells alone
*crIL-15 control did not function as expected
*crIL-15 control did not killed as expected

TABLE 7C

| Construct | Full nucleotide sequence |
|---|---|
| SB06251 | aagctttgctcttaggagtttcctaatacatcccaaactcaaatatataaagcatttgac |
| | ttgttctatgccctagggggcggggggaagctaagccagctttttttaacatttaaaatg |
| | ttaattccattttaaatgcacagatgtttttatttcataagggtttcaatgtgcatgaat |
| | gctgcaatattcctgttaccaaagctagtataaataaaaatagataaacgtggaaattac |
| | ttagagtttctgtcattaacgtttccttcctcagttgacaacataaatgcgctgctgaga |
| | agccagtttgcatctgtcaggatcaatttcccattatgccagtcatattaattactagtc |
| | aattagttgatttttatttttgacatatacatgtgaaagaccccacctgtaggtttggca |
| | agctagcttaagtaacgccattttgcaaggcatggaaaaatacataactgagaatagaaa |
| | agttcagatcaaggtcaggaacagatggaacagctgaatatgggccaaacaggatatctg |
| | tggtaagcagttcctgccccggctcagggccaagaacagatggaacagctgaatatgggc |
| | caaacaggatatctgtggtaagcagttcctgccccggctcagggccaagaacagatggtc |
| | cccagatgcggtccagccctcagcagtttctagagaaccatcagatgtttccagggtgcc |
| | ccaaggacctgaaatgaccctgtgccttatttgaactaaccaatcagttcgcttctcgct |
| | tctgttcgcgcgcttctgctccccgagctcaataaaagagcccacaacccctcactcggc |
| | gcgccagtcctccgattgactgagtcgcccgggtacccgtgtatccaataaaccctcttg |
| | cagttgcatccgacttgtggtctcgctgttccttgggagggtctcctctgagtgattgac |
| | tacccgtcagcgggggtctttcatttgggggctcgtccgagatcgggagacccctgccca |
| | gggaccaccgacccaccaccgggaggtaagctggccagcaacttatctgtgtctgtccga |
| | ttgtctagtgtctatgactgattttatgcgcctgcgtcggtactagttagctaactagct |
| | ctgtatctggcggacccgtggtggaactgacgagttcggaacacccggccgcaaccctgg |
| | gagacgtcccagggacttcgggggccgtttttgtggcccgacctgagtcctaaaatcccg |
| | atcgtttaggactctttggtgcaccccccttagaggagggatatgtggttctggtaggag |
| | acgagaacctaaaacagttcccgcctccgtctgaatttttgctttcggtttgggaccgaa |
| | gccgcgccgcgcgtcttgtctgctgcagcatcgttctgtgttgtctctgtctgactgtgt |
| | ttctgtatttgtctgaaaatatggatcttatatggggcacccccgccccttgtaaacttc |
| | cctgaccctgacatgacaagagttactaacagcccctctctccaagctcacttacaggct |
| | ctctacttagtccagcacgaagtctggagacctctggcggcagcctaccaagaacaactg |
| | gaccgaccggtggtacctcacccttaccgagtcggcgacacagtgtgggtccgccgacac |
| | cagactaagaacctagaacctcgctggaaaggaccttacacagtcctgctgaccaccccc |
| | accgccctcaaagtagacggcatcgcagcttggatacacgccgcccacgtgaaggctgcc |
| | gaccccgggggtggaccatcctctagactgccggatccGCCGCCACCATGCTGCTGCTGG |
| | TCACATCTCTGCTGCTGTGCGAGCTGCCCCATCCTGCCTTTCTGCTGATCCCTCACATGG |
| | ACATCGTGATGACACAGAGCCCCGATAGCCTGGCCGTGTCTCTGGGAGAAAGAGCCACCA |
| | TCAACTGCAAGAGCAGCCAGAGCCTGCTGTACTCCAGCAACCAGAAGAACTACCTGGCCT |
| | GGTATCAGCAAAAGCCCGGCCAGCCTCCTAAGCTGCTGATCTATTGGGCCAGCTCCAGAG |
| | AAAGCGGCGTGCCCGATAGATTTTCTGGCTCTGGCAGCGGCACCGACTTCACCCTGACAA |
| | TTTCTAGCCTGCAAGCCGAGGACGTGGCCGTGTACTACTGCCAGCAGTACTACAACTACC |
| | CTCTGACCTTCGGCCAGGGCACCAAGCTGGAAATCAAAGGCGGCGGAGGATCTGGCGGAG |
| | GTGGAAGTGGCGGAGGCGGATCTGAAGTGCAGCTGGTTGAATCAGGTGGCGGCCTGGTTC |
| | AACCTGGCGGATCTCTGAGACTGAGCTGTGCCGCCAGCGGCTTCACCTTCAACAAGAACG |
| | CCATGAACTGGGTCCGACAGGCCCCTGGCAAAGGCCTTGAATGGGTCGGACGGATCCGGA |
| | ACAAGACCAACAACTACGCCACCTACTACGCCGACAGCGTGAAGGCCAGATTCACCATCA |
| | GCCGGGACGACAGCAAGAACAGCCTGTACCTGCAGATGAACTCCCTGAAAACCGAGGACA |
| | CCGCCGTGTATTATTGCGTGGCCGGCAACAGCTTTGCCTACTGGGGACAGGGAACCCTGG |
| | TCACCGTGTCTGCCACAACAACCCCTGCTCCTAGACCTCCTACACCAGCTCCTACAATCG |
| | CCCTGCAGCCTCTGTCTCTGAGGCCAGAAGCTTGTAGACCAGCTGCTGGCGGAGCCGTGC |
| | ATACAAGAGGACTGGACTTCGCCTGTGATGTGGCCGCCATTCTCGGACTGGGACTTGTTC |
| | TGGGACTGCTGGGACCTCTGGCCATTCTGCTGGCTCTGTATCTGCTGCGGAGGGACCAAA |
| | GACTGCCTCCTGATGCTCACAAGCCTCCAGGCGGAGGCAGCTTCAGAACCCCTATCCAAG |
| | AGGAACAGGCCGACGCTCACAGCACCCTGGCCAAGATTAGAGTGAAGTTCAGCAGAAGCG |
| | CCGACGCACCCGCCTATAAGCAGGGACAGAACCAGCTGTACAACGAGCTGAACCTGGGGA |
| | GAAGAGAAGAGTACGACGTGCTGGACAAGCGGAGAGGCAGAGATCCTGAGATGGGCGGCA |
| | AGCCCAGACGGAAGAATCCTCAAGAGGGCCTGTATAATGAGCTGCAGAAAGACAAGATGG |
| | CCGAGGCCTACAGCGAGATCGGAATGAAGGGCGAGCGCAGAAGAGGCAAGGGACACGATG |
| | GACTGTACCAGGGCCTGAGCACCGCCACCAAGGATACCTATGATGCCCTGCACATGCAGG |
| | CCCTGCCTCCAAGAGGTAGCGGCCAGTGTACCAACTACGCCCTGCTGAAACTGGCCGGCG |
| | ACGTGGAATCTAATCCTGGACCTGGATCTGGCGAGGGACGCGGGAGTCTACTGACGTGTG |
| | GAGACGTGGAGGAAAACCCTGGACCTATGGACTGGACCTGGATCCTGTTTCTGGTGGCCG |
| | CTGCCACAAGAGTGCACAGCAATTGGGTCAACGTGATCAGCGACCTGAAGAAGATCGAGG |
| | ACCTGATCCAGAGCATGCACATCGACGCCACACTGTACACCGAGAGCGACGTGCACCCTA |
| | GCTGTAAAGTGACCGCCATGAAGTGCTTTCTGCTGGAACTGCAAGTGATCAGCCTGGAAA |
| | GCGGCGACGCCAGCATCCACGACACCGTGGAAAACCTGATCATCCTGGCCAACAACAGCC |
| | TGAGCAGCAACGGCAATGTGACCGAGTCCGGCTGCAAAGAGTGCGAGGAACTGGAAGAGA |
| | AGAATATCAAAGAGTTCCTGCAGAGCTTCGTGCACATCGTGCAGATGTTCATCAACACAA |
| | GCTCTGGCGGCGGAGGATCTGGCGGAGGTGGAAGCGGAGTTACACCCGAGCCTATCTTCA |
| | GCCTGATCGGAGGCGGTAGCGGAGGCGGAGGAAGTGGTGGCGGATCTCTGCAACTGCTGC |
| | CTAGCTGGGCCATCACACTGATCTCCGTGAACGGCATCTTCGTGATCTGCTGCCTGACCT |
| | ACTGCTTCGCCCCTAGATGCAGAGAGCGGCGGAGAAACGAACGGCTGAGAAGAGAATCTG |
| | TGCGGCCCGTTtaaagatctagatccggattagtccaatttgttaaagacaggatatcag |
| | tggtccaggctctagttttgactcaacaatatcaccagctgaagcctatagagtacgagc |
| | catagataaaataaaagattttatttagtctccagaaaaaggggggaatgaaagacccca |
| | cctgtaggtttggcaagctagcttaagtaacgccattttgcaaggcatggaaaaatacat |
| | aactgagaatagagaagttcagatcaaggtcaggaacagatggaacagctgaatatgggc |
| | caaacaggatatctgtggtaagcagttcctgccccggctcagggccaagaacagatggaa |
| | cagctgaatatgggccaaacaggatatctgtggtaagcagttcctgccccggctcagggc |
| | caagaacagatggtccccagatgcggtccagccctcagcagtttctagagaaccatcaga |
| | tgtttccagggtgccccaaggacctgaaatgaccctgtgccttatttgaactaaccaatc |
| | agttcgcttctcgcttctgttcgcgcgcttctgctccccgagctcaataaaagagcccac |
| | aacccctcactcggggcgccagtcctccgattgactgagtcgcccgggtacccgtgtatc |
| | caataaaccctcttgcagttgcatccgacttgtggtctcgctgttccttgggagggtctc |
| | ctctgagtgattgactacccgtcagcgggggtctttcacatgcagcatgtatcaaaatta |
| | atttggttttttttcttaagtatttacattaaatggccatagtacttaaagttacattgg |
| | cttccttgaaataaacatggagtattcagaatgtgtcataaatatttctaattttaagat |
| | agtatctccattggctttctactttttcttttatttttttttgtcctctgtcttccattt |
| | gttgttgttgttgtttgtttgtttgtttgttggttggttggttaatttttttttaaagat |
| | cctacactatagttcaagctagactattagctactctgtaacccagggtgaccttgaagt |
| | catgggtagcctgctgttttagccttcccacatctaagattacaggtatgagctatcatt |
| | tttggtatattgattgattgattgattgatgtgtgtgtgtgtgattgtgtttgtgtgtgt |
| | gaTtgtgTaTatgtgtgtatggTtgtgtgtgaTtgtgtgtatgtatgTTtgtgtgtgaTt |
| | gTgtgtgtgtgaTtgtgcatgtgtgtgtgtgtgaTtgtgtTtatgtgtatgaTtgtgtgt |
| | gtgtgtgtgtgtgtgtgtgtgtgtgtgtgtgtgtgtgtgttgtgTaTaTatatttatggt |
| | agtgagagGcaacgctccggctcaggtgtcaggttggtttttgagacagagtctttcact |
| | tagcttggaattaattcactggccgtcgttttacaacgtcgtgactgggaaaaccctggc |
| | gttacccaacttaatcgccttgcagcacatccccctttcgccagctggcgtaatagcgaa |
| | gaggcccgcaccgatcgcccttcccaacagttgcgcagcctgaatggcgaatggcgcctg |
| | atgcggtattttctccttacgcatctgtgcggtatttcacaccgcatatggtgcactctc |
| | agtacaatctgctctgatgccgcatagttaagccagccccgacacccgccaacacccgct |
| | gacgcgccctgacgggcttgtctgctcccggcatccgcttacagacaagctgtgaccgtc |
| | tccgggagctgcatgtgtcagaggttttcaccgtcatcaccgaaacgcgcgagacgaaag |
| | ggcctcgtgatacgcctatttttataggttaatgtcatgataataatggtttcttagacg |
| | tcaggtggcacttttcggggaaatgtgcgcggaacccctatttgtttatttttctaaata |
| | cattcaaatatgtatccgctcatgagacaataaccctgataaatgcttcaataatattga |
| | aaaaggaagagtatgagtattcaacatttccgtgtcgcccttattcccttttttgcggca |
| | ttttgccttcctgtttttgctcacccagaaacgctggtgaaagtaaaagatgctgaagat |
| | cagttgggtgcacgagtgggttacatcgaactggatctcaacagcggtaagatccttgag |
| | agttttcgccccgaagaacgttttccaatgatgagcacttttaaagttctgctatgtggc |
| | gcggtattatcccgtattgacgccgggcaagagcaactcggtcgccgcatacactattct |
| | cagaatgacttggttgagtactcaccagtcacagaaaagcatcttacggatggcatgaca |
| | gtaagagaattatgcagtgctgccataaccatgagtgataacactgcggccaacttactt |
| | ctgacaacgatcggaggaccgaaggagctaaccgcttttttgcacaacatgggggatcat |
| | gtaactcgccttgatcgttgggaaccggagctgaatgaagccataccaaacgacgagcgt |
| | gacaccacgatgcctgtagcaatggcaacaacgttgcgcaaactattaactggcgaacta |
| | cttactctagcttcccggcaacaattaatagactggatggaggcggataaagttgcagga |
| | ccacttctgcgctcggcccttccggctggctggtttattgctgataaatctggagccggt |
| | gagcgtgggtctcgcggtatcattgcagcactggggccagatggtaagccctcccgtatc |
| | gtagttatctacacgacggggagtcaggcaactatggatgaacgaaatagacagatcgct |
| | gagataggtgcctcactgattaagcattggtaactgtcagaccaagtttactcatatata |
| | ctttagattgatttaaaacttcatttttaatttaaaaggatctaggtgaagatccttttt |
| | gataatctcatgaccaaaatcccttaacgtgagttttcgttccactgagcgtcagacccc |
| | gtagaaaagatcaaaggatcttcttgagatcctttttttctgcgcgtaatctgctgcttg |
| | caaacaaaaaaaccaccgctaccagcggtggtttgtttgccggatcaagagctaccaact |
| | ctttttccgaaggtaactggcttcagcagagcgcagataccaaatactgtccttctagtg |
| | tagccgtagttaggccaccacttcaagaactctgtagcaccgcctacatacctcgctctg |
| | ctaatcctgttaccagtggctgctgccagtggcgataagtcgtgtcttaccgggttggac |
| | tcaagacgatagttaccggataaggcgcagcggtcgggctgaacggggggttcgtgcaca |
| | cagcccagcttggagcgaacgacctacaccgaactgagatacctacagcgtgagctatga |
| | gaaagcgccacgcttcccgaagggagaaaggcggacaggtatccggtaagcggcagggtc |
| | ggaacaggagagcgcacgagggagcttccagggggaaacgcctggtatctttatagtcct |
| | gtcgggtttcgccacctctgacttgagcgtcgatttttgtgatgctcgtcaggggggcgg |
| | agcctatggaaaaacgccagcaacgcggcctttttacggttcctggccttttgctggcct |
| | tttgctcacatgttctttcctgcgttatcccctgattctgtggataaccgtattaccgcc |
| | tttgagtgagctgataccgctcgccgcagccgaacgaccgagcgcagcgagtcagtgagc |
| | gaggaagcggaagagcgcccaatacgcaaaccgcctctccccgcgcgttggccgattcat |
| | taatgcagctggcacgacaggtttcccgactggaaagcgggcagtgagcgcaacgcaatt |
| | aatgtgagttagctcactcattaggcaccccaggctttacactttatgcttccggctcgt |
| | atgttgtgtggaattgtgagcggataacaatttcacacaggaaacagctatgaccatgat |
| | tacgcc (SEQ ID NO: 307) |
| SB06252 | aagctttgctcttaggagtttcctaatacatcccaaactcaaatatataaagcatttgac |
| | ttgttctatgccctagggggcggggggaagctaagccagctttttttaacatttaaaatg |
| | ttaattccattttaaatgcacagatgtttttatttcataagggtttcaatgtgcatgaat |
| | gctgcaatattcctgttaccaaagctagtataaataaaaatagataaacgtggaaattac |
| | ttagagtttctgtcattaacgtttccttcctcagttgacaacataaatgcgctgctgaga |
| | agccagtttgcatctgtcaggatcaatttcccattatgccagtcatattaattactagtc |
| | aattagttgatttttatttttgacatatacatgtgaaagaccccacctgtaggtttggca |
| | agctagcttaagtaacgccattttgcaaggcatggaaaaatacataactgagaatagaaa |
| | agttcagatcaaggtcaggaacagatggaacagctgaatatgggccaaacaggatatctg |
| | tggtaagcagttcctgccccggctcagggccaagaacagatggaacagctgaatatgggc |
| | caaacaggatatctgtggtaagcagttcctgccccggctcagggccaagaacagatggtc |
| | cccagatgcggtccagccctcagcagtttctagagaaccatcagatgtttccagggtgcc |
| | ccaaggacctgaaatgaccctgtgccttatttgaactaaccaatcagttcgcttctcgct |
| | tctgttcgcgcgcttctgctccccgagctcaataaaagagcccacaacccctcactcggc |
| | gcgccagtcctccgattgactgagtcgcccgggtacccgtgtatccaataaaccctcttg |
| | cagttgcatccgacttgtggtctcgctgttccttgggagggtctcctctgagtgattgac |
| | tacccgtcagcgggggtctttcatttgggggctcgtccgagatcgggagacccctgccca |
| | gggaccaccgacccaccaccgggaggtaagctggccagcaacttatctgtgtctgtccga |
| | ttgtctagtgtctatgactgattttatgcgcctgcgtcggtactagttagctaactagct |
| | ctgtatctggcggacccgtggtggaactgacgagttcggaacacccggccgcaaccctgg |
| | gagacgtcccagggacttcgggggccgtttttgtggcccgacctgagtcctaaaatcccg |
| | atcgtttaggactctttggtgcaccccccttagaggagggatatgtggttctggtaggag |
| | acgagaacctaaaacagttcccgcctccgtctgaatttttgctttcggtttgggaccgaa |
| | gccgcgccgcgcgtcttgtctgctgcagcatcgttctgtgttgtctctgtctgactgtgt |
| | ttctgtatttgtctgaaaatatggatcttatatggggcacccccgccccttgtaaacttc |
| | cctgaccctgacatgacaagagttactaacagcccctctctccaagctcacttacaggct |
| | ctctacttagtccagcacgaagtctggagacctctggcggcagcctaccaagaacaactg |
| | gaccgaccggtggtacctcacccttaccgagtcggcgacacagtgtgggtccgccgacac |
| | cagactaagaacctagaacctcgctggaaaggaccttacacagtcctgctgaccaccccc |
| | accgccctcaaagtagacggcatcgcagcttggatacacgccgcccacgtgaaggctgcc |
| | gaccccgggggtggaccatcctctagactgccggatccGCCGCCACCATGCTGCTGCTGG |
| | TCACATCTCTGCTGCTGTGCGAGCTGCCCCATCCTGCCTTTCTGCTGATCCCTCACATGG |
| | AAGTGCAGCTGGTGGAATCTGGCGGAGGACTGGTTCAACCTGGCGGCTCTCTGAGACTGT |
| | CTTGTGCCGCCAGCGGCTTCACCTTCAACAAGAACGCCATGAACTGGGTCCGACAGGCCC |
| | CTGGCAAAGGCCTTGAATGGGTCGGACGGATCCGGAACAAGACCAACAACTACGCCACCT |
| | ACTACGCCGACAGCGTGAAGGCCAGGTTCACCATCTCCAGAGATGACAGCAAGAACAGCC |
| | TGTACCTGCAGATGAACTCCCTGAAAACCGAGGACACCGCCGTGTACTATTGCGTGGCCG |
| | GCAATAGCTTTGCCTACTGGGGACAGGGCACCCTGGTTACAGTTTCTGCTGGCGGCGGAG |
| | GAAGCGGAGGCGGAGGATCCGGTGGTGGTGGATCTGACATCGTGATGACACAGAGCCCCG |
| | ATAGCCTGGCCGTGTCTCTGGGAGAAAGAGCCACCATCAACTGCAAGAGCAGCCAGAGCC |
| | TGCTGTACTCCAGCAACCAGAAGAACTACCTGGCCTGGTATCAGCAAAAGCCCGGCCAGC |
| | CTCCTAAGCTGCTGATCTATTGGGCCAGCTCCAGAGAAAGCGGCGTGCCCGATAGATTTT |
| | CTGGCTCTGGCAGCGGCACCGACTTCACCCTGACAATTTCTAGCCTGCAAGCCGAGGACG |
| | TGGCCGTGTATTACTGCCAGCAGTACTACAACTACCCTCTGACCTTCGGCCAGGGCACCA |
| | AGCTGGAAATCAAATCTGGCGCCCTGAGCAACAGCATCATGTACTTCAGCCACTTCGTGC |
| | CCGTGTTTCTGCCCGCCAAGCCTACAACAACCCCTGCTCCTAGACCTCCTACACCAGCTC |
| | CTACAATCGCCAGCCAGCCTCTGTCTCTGAGGCCAGAAGCTTGTAGACCTGCTGCAGGCG |
| | GAGCCGTGCATACAAGAGGACTGGATTTCGCCTGCGACATCTACATCTGGGCCCCTCTGG |
| | CTGGAACATGTGGTGTCCTGCTGCTGAGCCTGGTCATCACCCTGTACTGCAACCACCGGC |
| | GGAGCAAGAGAAGCAGACTGCTGCACAGCGACTACATGAACATGACCCCTAGACGGCCCG |
| | GACCTACCAGAAAGCACTACCAGCCTTACGCTCCTCCTAGAGACTTCGCCGCCTACCGGT |
| | CCAGAGTGAAGTTCAGCAGATCCGCCGATGCTCCCGCCTATCAGCAGGGACAGAACCAGC |
| | TGTACAACGAGCTGAACCTGGGGAGAAGAGAAGAGTACGACGTGCTGGACAAGCGGAGAG |
| | GCAGAGATCCTGAGATGGGCGGCAAGCCCAGACGGAAGAATCCTCAAGAGGGCCTGTATA |
| | ATGAGCTGCAGAAAGACAAGATGGCCGAGGCCTACAGCGAGATCGGAATGAAGGGCGAGC |
| | GCAGAAGAGGCAAGGGACACGATGGACTGTACCAGGGCCTGAGCACCGCCACCAAGGATA |
| | CCTATGATGCCCTGCACATGCAGGCCCTGCCTCCAAGAGGTAGCGGCCAGTGTACCAACT |
| | ACGCCCTGCTGAAACTGGCCGGCGACGTGGAATCTAATCCTGGACCTGGATCTGGCGAGG |
| | GACGCGGGAGTCTACTGACGTGTGGAGACGTGGAGGAAAACCCTGGACCTATGGACTGGA |
| | CCTGGATCCTGTTTCTGGTGGCCGCTGCCACAAGAGTGCACAGCAATTGGGTCAACGTGA |
| | TCAGCGACCTGAAGAAGATCGAGGACCTGATCCAGAGCATGCACATCGACGCCACACTGT |
| | ACACCGAGAGCGACGTGCACCCTAGCTGTAAAGTGACCGCCATGAAGTGCTTTCTGCTGG |
| | AACTGCAAGTGATCAGCCTGGAAAGCGGCGACGCCAGCATCCACGACACCGTGGAAAACC |
| | TGATCATCCTGGCCAACAACAGCCTGAGCAGCAACGGCAATGTGACCGAGTCCGGCTGCA |
| | AAGAGTGCGAGGAACTGGAAGAGAAGAATATCAAAGAGTTCCTGCAGAGCTTCGTGCACA |
| | TCGTGCAGATGTTCATCAACACAAGCTCTGGCGGCGGAGGATCTGGCGGAGGTGGAAGCG |
| | GAGTTACACCCGAGCCTATCTTCAGCCTGATCGGAGGCGGTAGCGGAGGCGGAGGAAGTG |
| | GTGGCGGATCTCTGCAACTGCTGCCTAGCTGGGCCATCACACTGATCTCCGTGAACGGCA |
| | TCTTCGTGATCTGCTGCCTGACCTACTGCTTCGCCCCTAGATGCAGAGAGCGGCGGAGAA |
| | ACGAACGGCTGAGAAGAGAATCTGTGCGGCCCGTTtaaagatctagatccggattagtcc |
| | aatttgttaaagacaggatatcagtggtccaggctctagttttgactcaacaatatcacc |
| | agctgaagcctatagagtacgagccatagataaaataaaagattttatttagtctccaga |
| | aaaaggggggaatgaaagaccccacctgtaggtttggcaagctagcttaagtaacgccat |
| | tttgcaaggcatggaaaaatacataactgagaatagagaagttcagatcaaggtcaggaa |
| | cagatggaacagctgaatatgggccaaacaggatatctgtggtaagcagttcctgccccg |
| | gctcagggccaagaacagatggaacagctgaatatgggccaaacaggatatctgtggtaa |
| | gcagttcctgccccggctcagggccaagaacagatggtccccagatgcggtccagccctc |
| | agcagtttctagagaaccatcagatgtttccagggtgccccaaggacctgaaatgaccct |
| | gtgccttatttgaactaaccaatcagttcgcttctcgcttctgttcgcgcgcttctgctc |
| | cccgagctcaataaaagagcccacaacccctcactcggggcgccagtcctccgattgact |
| | gagtcgcccgggtacccgtgtatccaataaaccctcttgcagttgcatccgacttgtggt |
| | ctcgctgttccttgggagggtctcctctgagtgattgactacccgtcagcgggggtcttt |
| | cacatgcagcatgtatcaaaattaatttggttttttttcttaagtatttacattaaatgg |
| | ccatagtacttaaagttacattggcttccttgaaataaacatggagtattcagaatgtgt |
| | cataaatatttctaattttaagatagtatctccattggctttctactttttcttttattt |
| | ttttttgtcctctgtcttccatttgttgttgttgttgtttgtttgtttgtttgttggttg |
| | gttggttaatttttttttaaagatcctacactatagttcaagctagactattagctactc |
| | tgtaacccagggtgaccttgaagtcatgggtagcctgctgttttagccttcccacatcta |
| | agattacaggtatgagctatcatttttggtatattgattgattgattgattgatgtgtgt |
| | gtgtgtgattgtgtttgtgtgtgtgaTtgtgTaTatgtgtgtatggTtgtgtgtgaTtgt |
| | gtgtatgtatgTTtgtgtgtgaTtgTgtgtgtgtgaTtgtgcatgtgtgtgtgtgtgaTt |
| | gtgtTtatgtgtatgaTtgtgtgtgtgtgtgtgtgtgtgtgtgtgtgtgtgtgtgtgtgt |
| | gtgttgtgTaTaTatatttatggtagtgagagGcaacgctccggctcaggtgtcaggttg |
| | gtttttgagacagagtctttcacttagcttggaattaattcactggccgtcgttttacaa |
| | cgtcgtgactgggaaaaccctggcgttacccaacttaatcgccttgcagcacatccccct |
| | ttcgccagctggcgtaatagcgaagaggcccgcaccgatcgcccttcccaacagttgcgc |
| | agcctgaatggcgaatggcgcctgatgcggtattttctccttacgcatctgtgcggtatt |
| | tcacaccgcatatggtgcactctcagtacaatctgctctgatgccgcatagttaagccag |
| | ccccgacacccgccaacacccgctgacgcgccctgacgggcttgtctgctcccggcatcc |
| | gcttacagacaagctgtgaccgtctccgggagctgcatgtgtcagaggttttcaccgtca |
| | tcaccgaaacgcgcgagacgaaagggcctcgtgatacgcctatttttataggttaatgtc |
| | atgataataatggtttcttagacgtcaggtggcacttttcggggaaatgtgcgcggaacc |
| | cctatttgtttatttttctaaatacattcaaatatgtatccgctcatgagacaataaccc |
| | tgataaatgcttcaataatattgaaaaaggaagagtatgagtattcaacatttccgtgtc |
| | gcccttattcccttttttgcggcattttgccttcctgtttttgctcacccagaaacgctg |
| | gtgaaagtaaaagatgctgaagatcagttgggtgcacgagtgggttacatcgaactggat |
| | ctcaacagcggtaagatccttgagagttttgccccgaagaacgttttccaatgatgagca |
| | cttttaaagttctgctatgtggcgcggtattatcccgtattgacgccgggcaagagcaac |
| | tcggtcgccgcatacactattctcagaatgacttggttgagtactcaccagtcacagaaa |
| | agcatcttacggatggcatgacagtaagagaattatgcagtgctgccataaccatgagtg |
| | ataacactgcggccaacttacttctgacaacgatcggaggaccgaaggagctaaccgctt |
| | ttttgcacaacatgggggatcatgtaactcgccttgatcgttgggaaccggagctgaatg |
| | aagccataccaaacgacgagcgtgacaccacgatgcctgtagcaatggcaacaacgttgc |
| | gcaaactattaactggcgaactacttactctagcttcccggcaacaattaatagactgga |
| | tggaggcggataaagttgcaggaccacttctgcgctcggcccttccggctggctggttta |
| | ttgctgataaatctggagccggtgagcgtgggtctcgcggtatcattgcagcactggggc |
| | cagatggtaagccctcccgtatcgtagttatctacacgacggggagtcaggcaactatgg |
| | atgaacgaaatagacagatcgctgagataggtgcctcactgattaagcattggtaactgt |
| | cagaccaagtttactcatatatactttagattgatttaaaacttcatttttaatttaaaa |
| | ggatctaggtgaagatcctttttgataatctcatgaccaaaatcccttaacgtgagtttt |
| | cgttccactgagcgtcagaccccgtagaaaagatcaaaggatcttcttgagatccttttt |
| | ttctgcgcgtaatctgctgcttgcaaacaaaaaaaccaccgctaccagcggtggtttgtt |
| | tgccggatcaagagctaccaactctttttccgaaggtaactggcttcagcagagcgcaga |
| | taccaaatactgtccttctagtgtagccgtagttaggccaccacttcaagaactctgtag |
| | caccgcctacatacctcgctctgctaatcctgttaccagtggctgctgccagtggcgata |
| | agtcgtgtcttaccgggttggactcaagacgatagttaccggataaggcgcagcggtcgg |
| | gctgaacggggggttcgtgcacacagcccagcttggagcgaacgacctacaccgaactga |
| | gatacctacagcgtgagctatgagaaagcgccacgcttcccgaagggagaaaggcggaca |
| | ggtatccggtaagcggcagggtcggaacaggagagcgcacgagggagcttccagggggaa |
| | acgcctggtatctttatagtcctgtcgggtttcgccacctctgacttgagcgtcgatttt |
| | tgtgatgctcgtcaggggggcggagcctatggaaaaacgccagcaacgcggcctttttac |
| | ggttcctggccttttgctggccttttgctcacatgttctttcctgcgttatcccctgatt |
| | ctgtggataaccgtattaccgcctttgagtgagctgataccgctcgccgcagccgaacga |
| | ccgagcgcagcgagtcagtgagcgaggaagcggaagagcgcccaatacgcaaaccgcctc |
| | tccccgcgcgttggccgattcattaatgcagctggcacgacaggtttcccgactggaaag |
| | cgggcagtgagcgcaacgcaattaatgtgagttagctcactcattaggcaccccaggctt |
| | tacactttatgcttccggctcgtatgttgtgtggaattgtgagcggataacaatttcaca |
| | caggaaacagctatgaccatgattacgcc (SEQ ID NO: 308) |
| SB06257 | aagcttgaattcgagcttgcatgcctgcaggtcgttacataacttacggtaaatggcccg |
| (GM-CSF-Ra | cctggctgaccgcccaacgacccccgcccattgacgtcaataatgacgtatgttcccata |
| (SS)-aGPC3 | gtaacgccaatagggactttccattgacgtcaatgggtggagtatttacggtaaactgcc |
| hPY7 vL- | cacttggcagtacatcaagtgtatcatatgccaagtacgccccctattgacgtcaatgac |
| (GGGGS)3 | ggtaaatggcccgcctggcattatgcccagtacatgaccttatgggactttcctacttgg |
| (SEQ ID NO: | cagtacatctacgtattagtcatcgctattaccatggtgatgcggttttggcagtacatc |
| 223)-aGPC3 | aatgggcgtggatagcggtttgactcacggggatttccaagtctccaccccattgacgtc |
| hPY7 vH- | aatgggagtttgttttggcaccaaaatcaacgggactttccaaaatgtcgtaacaactcc |
| CD8 S2L | gccccattgacgcaaatgggggtaggcgtgtacggtgggaggtctatataagcagagctc |
| (Hinge)- | aataaaagagcccacaacccctcactcggcgcgccagtcctccgattgactgagtcgccc |
| OX40 (TM)- | gggtacccgtgtatccaataaaccctcttgcagttgcatccgacttgtggtctcgctgtt |
| OX40 (ICD)- | ccttgggagggtctcctctgagtgattgactacccgtcagcgggggtctttcatttgggg |
| CD3z (ICD)- | gctcgtccgagatcgggagacccctgcccagggaccaccgacccaccaccgggaggtaag |
| E2A T2A-IgE | ctggccagcaacttatctgtgtctgtccgattgtctagtgtctatgactgattttatgcg |
| (SS)-IL 15- | cctgcgtcggtactagttagctaactagctctgtatctggcggacccgtggtggaactga |
| Tace10 | cgagttcggaacacccggccgcaaccctgggagacgtcccagggacttcgggggccgttt |
| (cleavage | ttgtggcccgacctgagtcctaaaatcccgatcgtttaggactctttggtgcacccccct |
| site)-B7-1 | tagaggagggatatgtggttctggtaggagacgagaacctaaaacagttcccgcctccgt |
| (TM)) | ctgaatttttgctttcggtttgggaccgaagccgcgccgcgcgtcttgtctgctgcagca |
| | tcgttctgtgttgtctctgtctgactgtgtttctgtatttgtctgaaaatatgggccccc |
| | cctcgaggtaacgccattttgcaaggcatggaaaaataccaaaccaagaatagagaagtt |
| | cagatcaagggcgggtacatgaaaatagctaacgttgggccaaacaggatatctgcggtg |
| | agcagtttcggccccggcccggggccaagaacagatggtcaccgcagtttcggccccggc |
| | ccgaggccaagaacagatggtccccagatatggcccaaccctcagcagtttcttaagacc |
| | catcagatgtttccaggctcccccaaggacctgaaatgaccctgcgccttatttgaatta |
| | accaatcagcctgcttctcgcttctgttcgcgcgcttctgcttcccgagctctataaaag |
| | agctcacaacccctcactcggcgcgccagtcctccgacagactgagtcgcccgggGCCGC |
| | CACCATGCTGCTGCTGGTCACATCTCTGCTGCTGTGCGAGCTGCCCCATCCTGCCTTTCT |
| | GCTGATCCCTCACATGGACATCGTGATGACACAGAGCCCCGATAGCCTGGCCGTGTCTCT |
| | GGGAGAAAGAGCCACCATCAACTGCAAGAGCAGCCAGAGCCTGCTGTACTCCAGCAACCA |
| | GAAGAACTACCTGGCCTGGTATCAGCAAAAGCCCGGCCAGCCTCCTAAGCTGCTGATCTA |
| | TTGGGCCAGCTCCAGAGAAAGCGGCGTGCCCGATAGATTTTCTGGCTCTGGCAGCGGCAC |
| | CGACTTCACCCTGACAATTTCTAGCCTGCAAGCCGAGGACGTGGCCGTGTACTACTGCCA |
| | GCAGTACTACAACTACCCTCTGACCTTCGGCCAGGGCACCAAGCTGGAAATCAAAGGCGG |
| | CGGAGGATCTGGCGGAGGTGGAAGTGGCGGAGGCGGATCTGAAGTGCAGCTGGTTGAATC |
| | AGGTGGCGGCCTGGTTCAACCTGGCGGATCTCTGAGACTGAGCTGTGCCGCCAGCGGCTT |
| | CACCTTCAACAAGAACGCCATGAACTGGGTCCGACAGGCCCCTGGCAAAGGCCTTGAATG |
| | GGTCGGACGGATCCGGAACAAGACCAACAACTACGCCACCTACTACGCCGACAGCGTGAA |
| | GGCCAGATTCACCATCAGCCGGGACGACAGCAAGAACAGCCTGTACCTGCAGATGAACTC |
| | CCTGAAAACCGAGGACACCGCCGTGTATTATTGCGTGGCCGGCAACAGCTTTGCCTACTG |
| | GGGACAGGGAACCCTGGTCACCGTGTCTGCCACAACAACCCCTGCTCCTAGACCTCCTAC |
| | ACCAGCTCCTACAATCGCCCTGCAGCCTCTGTCTCTGAGGCCAGAAGCTTGTAGACCAGC |
| | TGCTGGCGGAGCCGTGCATACAAGAGGACTGGACTTCCCTGTGATGTGGCCGCCATTCTC |
| | GGACTGGGACTTGTTCTGGGACTGCTGGGACCTCTGGCCATTCTGCTGGCTCTGTATCTG |
| | CTGCGGAGGGACCAAAGACTGCCTCCTGATGCTCACAAGCCTCCAGGCGGAGGCAGCTTC |
| | AGAACCCCTATCCAAGAGGAACAGGCCGACGCTCACAGCACCCTGGCCAAGATTAGAGTG |
| | AAGTTCAGCAGAAGCGCCGACGCACCCGCCTATAAGCAGGGACAGAACCAGCTGTACAAC |
| | GAGCTGAACCTGGGGAGAAGAGAAGAGTACGACGTGCTGGACAAGCGGAGAGGCAGAGAT |
| | CCTGAGATGGGCGGCAAGCCCAGACGGAAGAATCCTCAAGAGGGCCTGTATAATGAGCTG |
| | CAGAAAGACAAGATGGCCGAGGCCTACAGCGAGATCGGAATGAAGGGCGAGCGCAGAAGA |
| | GGCAAGGGACACGATGGACTGTACCAGGGCCTGAGCACCGCCACCAAGGATACCTATGAT |
| | GCCCTGCACATGCAGGCCCTGCCTCCAAGAGGTAGCGGCCAGTGTACCAACTACGCCCTG |
| | CTGAAACTGGCCGGCGACGTGGAATCTAATCCTGGACCTGGATCTGGCGAGGGACGCGGG |
| | AGTCTACTGACGTGTGGAGACGTGGAGGAAAACCCTGGACCTATGGACTGGACCTGGATC |
| | CTGTTTCTGGTGGCCGCTGCCACAAGAGTGCACAGCAATTGGGTCAACGTGATCAGCGAC |
| | CTGAAGAAGATCGAGGACCTGATCCAGAGCATGCACATCGACGCCACACTGTACACCGAG |
| | AGCGACGTGCACCCTAGCTGTAAAGTGACCGCCATGAAGTGCTTTCTGCTGGAACTGCAA |
| | GTGATCAGCCTGGAAAGCGGCGACGCCAGCATCCACGACACCGTGGAAAACCTGATCATC |
| | CTGGCCAACAACAGCCTGAGCAGCAACGGCAATGTGACCGAGTCCGGCTGCAAAGAGTGC |
| | GAGGAACTGGAAGAGAAGAATATCAAAGAGTTCCTGCAGAGCTTCGTGCACATCGTGCAG |
| | ATGTTCATCAACACAAGCTCTGGCGGCGGAGGATCTGGCGGAGGTGGAAGCGGAGTTACA |
| | CCCGAGCCTATCTTCAGCCTGATCGGAGGCGGTAGCGGAGGCGGAGGAAGTGGTGGCGGA |
| | TCTCTGCAACTGCTGCCTAGCTGGGCCATCACACTGATCTCCGTGAACGGCATCTTCGTG |
| | ATCTGCTGCCTGACCTACTGCTTCGCCCCTAGATGCAGAGAGCGGCGGAGAAACGAACGG |
| | CTGAGAAGAGAATCTGTGCGGCCCGTTtaaggatccggattagtccaatttgttaaagac |
| | aggatgggctgcaggaattccgataatcaacctctggattacaaaatttgtgaaagattg |
| | actggtattcttaactatgttgctccttttacgctatgtggatacgctgctttaatgcct |
| | ttgtatcatgctattgcttcccgtatggctttcattttctcctccttgtataaatcctgg |
| | ttgctgtctctttatgaggagttgtggcccgttgtcaggcaacgtggcgtggtgtgcact |
| | gtgtttgctgacgcaacccccactggttggggcattgccaccacctgtcagctcctttcc |
| | gggactttcgctttccccctccctattgccacggcggaactcatcgccgcctgccttgcc |
| | cgctgctggacaggggctcggctgttgggcactgacaattccgtggtgttgtcggggaag |
| | ctgacgtcctttccatggctgctcgcctgtgttgccacctggattctgcgcgggacgtcc |
| | ttctgctacgtcccttcggccctcaatccagcggaccttccttcccgcggcctgctgccg |
| | gctctgcggcctcttccgcgtcttcgccttcgccctcagacgagtcggatctccctttgg |
| | gccgcctccccgcctggagaattcgatatcagtggtccaggctctagttttgactcaaca |
| | atatcaccagctgaagcctatagagtacgagccatagataaaataaaagattttatttag |
| | tctccagaaaaaggggggaatgaaagaccccacctgtaggtttggcaagctagcaataaa |
| | agagcccacaacccctcactcggggcgccagtcctccgattgactgagtcgcccggccgc |
| | ttcgagcagacatgataagatacattgatgagtttggacaaaccacaactagaatgcagt |
| | gaaaaaaatgctttatttgtgaaatttgtgatgctattgctttatttgtaaccattataa |
| | gctgcaataaacaagttaacaacaacaattgcattcattttatgtttcaggttcaggggg |
| | agatgtgggaggttttttaaagcaagtaaaacctctacaaatgtggtaaaatcgataagg |
| | atcgggtacccgtgtatccaataaaccctcttgcagttgcatccgacttgtggtctcgct |
| | gttccttgggagggtctcctctgagtgattgactacccgtcagcgggggtctttcacaca |
| | tgcagcatgtatcaaaattaatttggttttttttcttaagctgtgccttctagttgccag |
| | ccatctgttgtttgcccctcccccgtgccttccttgaccctggaaggtgccactcccact |
| | gtcctttcctaataaaatgaggaaattgcatcgcattgtctgagtaggtgtcattctatt |
| | ctggggggtggggggggcaggacagcaagggggaggattgggaagacaatagcaggcatg |
| | ctggggatgcggtgggctctatggagatcccgcggtacctcgcgaatgcatctagatcca |
| | atggcctttttggcccagacatgataagatacattgatgagtttggacaaaccacaacta |
| | gaatgcagtgaaaaaaatgctttatttgtgaaatttgtgatgctattgctttatttgtaa |
| | ccattataagGctgcaataaacaagttgcggccgcttagccctcccacacataaccagag |
| | ggcagcaattcacgaatcccaactgccgtcggctgtccatcactgtccttcactatggct |
| | ttgatcccaggatgcagatcgagaagcacctgtcggcaccgtccgcaggggctcaagatg |
| | cccctgttctcatttccgatcgcgacgatacaagtcaggttgccagctgccgcagcagca |
| | gcagtgcccagcaccacgagttctgcacaaggtcccccagtaaaatgatatacattgaca |
| | ccagtgaagatgcggccgtcgctagagagagctgcgctggcgacgctgtagtcttcagag |
| | atggggatgctgttgattgtagccgttgctctttcaatgagggtggattcttcttgagac |
| | aaaggcttggccatgcggccgccgctcggtgttcgaggccacacgcgtcaccttaatatg |
| | cgaagtggacctcggaccgcgccgccccgactgcatctgcgtgttcgaattcgccaatga |
| | caagacgctgggggggtttgtgtcatcatagaactaaagacatgcaaatatatttcttcc |
| | ggggggtaccggcctttttggccATTGGatcggatctggccaaaaaggcccttaagtatt |
| | tacattaaatggccatagtacttaaagttacattggcttccttgaaataaacatggagta |
| | ttcagaatgtgtcataaatatttctaattttaagatagtatctccattggctttctactt |
| | tttcttttatttttttttgtcctctgtcttccatttgttgttgttgttgtttgtttgttt |
| | gtttgttggttggttggttaatttttttttaaagatcctacactatagttcaagctagac |
| | tattagctactctgtaacccagggtgaccttgaagtcatgggtagcctgctgttttagcc |
| | ttcccacatctaagattacaggtatgagctatcatttttggtatattgattgattgattg |
| | attgatgtgtgtgtgtgtgattgtgtttgtgtgtgtgaTtgtgTaTatgtgtgtatggTt |
| | gtgtgtgaTtgtgtgtatgtatgTTtgtgtgtgaTtgTgtgtgtgtgaTtgtgcatgtgt |
| | gtgtgtgtgaTtgtgtTtatgtgtatgaTtgtgtgtgtgtgtgtgtgtgtgtgtgtgtgt |
| | gtgtgtgtgtgtgtgttgtgTaTaTatatttatggtagtgagagGcaacgctccggctca |
| | ggtgtcaggttggtttttgagacagagtctttcacttagcttggaattcactggccgtcg |
| | ttttacaacgtcgtgactgggaaaaccctggcgttacccaacttaatcgccttgcagcac |
| | atccccctttcgccagctggcgtaatagcgaagaggcccgcaccgatcgcccttcccaac |
| | agttgcgcagcctgaatggcgaatggcgcctgatgcggtattttctccttacgcatctgt |
| | gcggtatttcacaccgcatatggtgcactctcagtacaatctgctctgatgccgcatagt |
| | taagccagccccgacacccgccaacacccgctgacgcgccctgacgggcttgtctgctcc |
| | cggcatccgcttacagacaagctgtgaccgtctccgggagctgcatgtgtcagaggtttt |
| | caccgtcatcaccgaaacgcgcgagacgaaagggcctcgtgatacgcctatttttatagg |
| | ttaatgtcatgataataatggtttcttagacgtcaggtggcacttttcggggaaatgtgc |
| | gcggaacccctatttgtttatttttctaaatacattcaaatatgtatccgctcatgagac |
| | aataaccctgataaatgcttcaataatattgaaaaaggaagagtatgagtattcaacatt |
| | tccgtgtcgcccttattcccttttttgcggcattttgccttcctgtttttgctcacccag |
| | aaacgctggtgaaagtaaaagatgctgaagatcagttgggtgcacgagtgggttacatcg |
| | aactggatctcaacagcggtaagatccttgagagttttcgccccgaagaacgttttccaa |
| | tgatgagcacttttaaagttctgctatgtggcgcggtattatcccgtattgacgccgggc |
| | aagagcaactcggtcgccgcatacactattctcagaatgacttggttgagtactcaccag |
| | tcacagaaaagcatcttacggatggcatgacagtaagagaattatgcagtgctgccataa |
| | ccatgagtgataacactgcggccaacttacttctgacaacgatcggaggaccgaaggagc |
| | taaccgcttttttgcacaacatgggggatcatgtaactcgccttgatcgttgggaaccgg |
| | agctgaatgaagccataccaaacgacgagcgtgacaccacgatgcctgtagcaatggcaa |
| | caacgttgcgcaaactattaactggcgaactacttactctagcttcccggcaacaattaa |
| | tagactggatggaggcggataaagttgcaggaccacttctgcgctcggcccttccggctg |
| | gctggtttattgctgataaatctggagccggtgagcgtgggtctcgcggtatcattgcag |
| | cactggggccagatggtaagccctcccgtatcgtagttatctacacgacggggagtcagg |
| | caactatggatgaacgaaatagacagatcgctgagataggtgcctcactgattaagcatt |
| | ggtaactgtcagaccaagtttactcatatatactttagattgatttaaaacttcattttt |
| | aatttaaaaggatctaggtgaagatcctttttgataatctcatgaccaaaatcccttaac |
| | gtgagttttcgttccactgagcgtcagaccccgtagaaaagatcaaaggatcttcttgag |
| | atcctttttttctgcgcgtaatctgctgcttgcaaacaaaaaaaccaccgctaccagcgg |
| | tggtttgtttgccggatcaagagctaccaactctttttccgaaggtaactggcttcagca |
| | gagcgcagataccaaatactgtccttctagtgtagccgtagttaggccaccacttcaaga |
| | actctgtagcaccgcctacatacctcgctctgctaatcctgttaccagtggctgctgcca |
| | gtggcgataagtcgtgtcttaccgggttggactcaagacgatagttaccggataaggcgc |
| | agcggtcgggctgaacggggggttcgtgcacacagcccagcttggagcgaacgacctaca |
| | ccgaactgagatacctacagcgtgagctatgagaaagcgccacgcttcccgaagggagaa |
| | aggcggacaggtatccggtaagcggcagggtcggaacaggagagcgcacgagggagcttc |
| | cagggggaaacgcctggtatctttatagtcctgtcgggtttcgccacctctgacttgagc |
| | gtcgatttttgtgatgctcgtcaggggggcggagcctatggaaaaacgccagcaacgcgg |
| | cctttttacggttcctggccttttgctggccttttgctcacatgttctttcctgcgttat |
| | cccctgattctgtggataaccgtattaccgcctttgagtgagctgataccgctcgccgca |
| | gccgaacgaccgagcgcagcgagtcagtgagcgaggaagcggaagagcgcccaatacgca |
| | aaccgcctctccccgcgcgttggccgattcattaatgcagctggcacgacaggtttcccg |
| | actggaaagcgggcagtgagcgcaacgcaattaatgtgagttagctcactcattaggcac |
| | cccaggctttacactttatgcttccggctcgtatgttgtgtggaattgtgagcggataac |
| | aatttcacacaggaaacagctatgaccatgattacgcc (SEQ ID NO: 309) |
| SB06258 | aagcttgaattcgagcttgcatgcctgcaggtcgttacataacttacggtaaatggcccg |
| (GM-CSF-Ra | cctggctgaccgcccaacgacccccgcccattgacgtcaataatgacgtatgttcccata |
| (SS)-aGPC3 | gtaacgccaatagggactttccattgacgtcaatgggtggagtatttacggtaaactgcc |
| hPY7 vH- | cacttggcagtacatcaagtgtatcatatgccaagtacgccccctattgacgtcaatgac |
| (GGGGS)3 | ggtaaatggcccgcctggcattatgcccagtacatgaccttatgggactttcctacttgg |
| (SEQ ID NO: | cagtacatctacgtattagtcatcgctattaccatggtgatgcggttttggcagtacatc |
| 223)-aGPC3 | aatgggcgtggatagcggtttgactcacggggatttccaagtctccaccccattgacgtc |
| hPY7 vL- | aatgggagtttgttttggcaccaaaatcaacgggactttccaaaatgtcgtaacaactcc |
| CD8FA | gccccattgacgcaaatgggggtaggcgtgtacggtgggaggtctatataagcagagctc |
| (Hinge)-CD8 | aataaaagagcccacaacccctcactcggcgcgccagtcctccgattgactgagtcgccc |
| (TM)-CD28 | gggtacccgtgtatccaataaaccctcttgcagttgcatccgacttgtggtctcgctgtt |
| (ICD)-CD3z | ccttgggagggtctcctctgagtgattgactacccgtcagcgggggtctttcatttgggg |
| (ICD)-E2A | gctcgtccgagatcgggagacccctgcccagggaccaccgacccaccaccgggaggtaag |
| T2A-IgE | ctggccagcaacttatctgtgtctgtccgattgtctagtgtctatgactgattttatgcg |
| (SS)-IL15- | cctgcgtcggtactagttagctaactagctctgtatctggcggacccgtggtggaactga |
| Tace10 | cgagttcggaacacccggccgcaaccctgggagacgtcccagggacttcgggggccgttt |
| (cleavage | ttgtggcccgacctgagtcctaaaatcccgatcgtttaggactctttggtgcacccccct |
| site)-B7-1 | tagaggagggatatgtggttctggtaggagacgagaacctaaaacagttcccgcctccgt |
| (TM)) | ctgaatttttgctttcggtttgggaccgaagccgcgccgcgcgtcttgtctgctgcagca |
| | tcgttctgtgttgtctctgtctgactgtgtttctgtatttgtctgaaaatatgggccccc |
| | cctcgaggtaacgccattttgcaaggcatggaaaaataccaaaccaagaatagagaagtt |
| | cagatcaagggcgggtacatgaaaatagctaacgttgggccaaacaggatatctgcggtg |
| | agcagtttcggccccggcccggggccaagaacagatggtcaccgcagtttcggccccggc |
| | ccgaggccaagaacagatggtccccagatatggcccaaccctcagcagtttcttaagacc |
| | catcagatgtttccaggctcccccaaggacctgaaatgaccctgcgccttatttgaatta |
| | accaatcagcctgcttctcgcttctgttcgcgcgcttctgcttcccgagctctataaaag |
| | agctcacaacccctcactcggcgcgccagtcctccgacagactgagtcgcccgggGCCGC |
| | CACCATGCTGCTGCTGGTCACATCTCTGCTGCTGTGCGAGCTGCCCCATCCTGCCTTTCT |
| | GCTGATCCCTCACATGGAAGTGCAGCTGGTGGAATCTGGCGGAGGACTGGTTCAACCTGG |
| | CGGCTCTCTGAGACTGTCTTGTGCCGCCAGCGGCTTCACCTTCAACAAGAACGCCATGAA |
| | CTGGGTCCGACAGGCCCCTGGCAAAGGCCTTGAATGGGTCGGACGGATCCGGAACAAGAC |
| | CAACAACTACGCCACCTACTACGCCGACAGCGTGAAGGCCAGGTTCACCATCTCCAGAGA |
| | TGACAGCAAGAACAGCCTGTACCTGCAGATGAACTCCCTGAAAACCGAGGACACCGCCGT |
| | GTACTATTGCGTGGCCGGCAATAGCTTTGCCTACTGGGGACAGGGCACCCTGGTTACAGT |
| | TTCTGCTGGCGGCGGAGGAAGCGGAGGCGGAGGATCCGGTGGTGGTGGATCTGACATCGT |
| | GATGACACAGAGCCCCGATAGCCTGGCCGTGTCTCTGGGAGAAAGAGCCACCATCAACTG |
| | CAAGAGCAGCCAGAGCCTGCTGTACTCCAGCAACCAGAAGAACTACCTGGCCTGGTATCA |
| | GCAAAAGCCCGGCCAGCCTCCTAAGCTGCTGATCTATTGGGCCAGCTCCAGAGAAAGCGG |
| | CGTGCCCGATAGATTTTCTGGCTCTGGCAGCGGCACCGACTTCACCCTGACAATTTCTAG |
| | CCTGCAAGCCGAGGACGTGGCCGTGTATTACTGCCAGCAGTACTACAACTACCCTCTGAC |
| | CTTCGGCCAGGGCACCAAGCTGGAAATCAAATCTGGCGCCCTGAGCAACAGCATCATGTA |
| | CTTCAGCCACTTCGTGCCCGTGTTTCTGCCCGCCAAGCCTACAACAACCCCTGCTCCTAG |
| | ACCTCCTACACCAGCTCCTACAATCGCCAGCCAGCCTCTGTCTCTGAGGCCAGAAGCTTG |
| | TAGACCTGCTGCAGGCGGAGCCGTGCATACAAGAGGACTGGATTTCGCCTGCGACATCTA |
| | CATCTGGGCCCCTCTGGCTGGAACATGTGGTGTCCTGCTGCTGAGCCTGGTCATCACCCT |
| | GTACTGCAACCACCGGCGGAGCAAGAGAAGCAGACTGCTGCACAGCGACTACATGAACAT |
| | GACCCCTAGACGGCCCGGACCTACCAGAAAGCACTACCAGCCTTACGCTCCTCCTAGAGA |
| | CTTCGCCGCCTACCGGTCCAGAGTGAAGTTCAGCAGATCCGCCGATGCTCCCGCCTATCA |
| | GCAGGGACAGAACCAGCTGTACAACGAGCTGAACCTGGGGAGAAGAGAAGAGTACGACGT |
| | GCTGGACAAGCGGAGAGGCAGAGATCCTGAGATGGGCGGCAAGCCCAGACGGAAGAATCC |
| | TCAAGAGGGCCTGTATAATGAGCTGCAGAAAGACAAGATGGCCGAGGCCTACAGCGAGAT |
| | CGGAATGAAGGGCGAGCGCAGAAGAGGCAAGGGACACGATGGACTGTACCAGGGCCTGAG |
| | CACCGCCACCAAGGATACCTATGATGCCCTGCACATGCAGGCCCTGCCTCCAAGAGGTAG |
| | CGGCCAGTGTACCAACTACGCCCTGCTGAAACTGGCCGGCGACGTGGAATCTAATCCTGG |
| | ACCTGGATCTGGCGAGGGACGCGGGAGTCTACTGACGTGTGGAGACGTGGAGGAAAACCC |
| | TGGACCTATGGACTGGACCTGGATCCTGTTTCTGGTGGCCGCTGCCACAAGAGTGCACAG |
| | CAATTGGGTCAACGTGATCAGCGACCTGAAGAAGATCGAGGACCTGATCCAGAGCATGCA |
| | CATCGACGCCACACTGTACACCGAGAGCGACGTGCACCCTAGCTGTAAAGTGACCGCCAT |
| | GAAGTGCTTTCTGCTGGAACTGCAAGTGATCAGCCTGGAAAGCGGCGACGCCAGCATCCA |
| | CGACACCGTGGAAAACCTGATCATCCTGGCCAACAACAGCCTGAGCAGCAACGGCAATGT |
| | GACCGAGTCCGGCTGCAAAGAGTGCGAGGAACTGGAAGAGAAGAATATCAAAGAGTTCCT |
| | GCAGAGCTTCGTGCACATCGTGCAGATGTTCATCAACACAAGCTCTGGCGGCGGAGGATC |
| | TGGCGGAGGTGGAAGCGGAGTTACACCCGAGCCTATCTTCAGCCTGATCGGAGGCGGTAG |
| | CGGAGGCGGAGGAAGTGGTGGCGGATCTCTGCAACTGCTGCCTAGCTGGGCCATCACACT |
| | GATCTCCGTGAACGGCATCTTCGTGATCTGCTGCCTGACCTACTGCTTCGCCCCTAGATG |
| | CAGAGAGCGGCGGAGAAACGAACGGCTGAGAAGAGAATCTGTGCGGCCCGTTtaaggatc |
| | cggattagtccaatttgttaaagacaggatgggctgcaggaattccgataatcaacctct |
| | ggattacaaaatttgtgaaagattgactggtattcttaactatgttgctccttttacgct |
| | atgtggatacgctgctttaatgcctttgtatcatgctattgcttcccgtatggctttcat |
| | tttctcctccttgtataaatcctggttgctgtctctttatgaggagttgtggcccgttgt |
| | caggcaacgtggcgtggtgtgcactgtgtttgctgacgcaacccccactggttggggcat |
| | tgccaccacctgtcagctcctttccgggactttcgctttccccctccctattgccacggc |
| | ggaactcatcgccgcctgccttgcccgctgctggacaggggctcggctgttgggcactga |
| | caattccgtggtgttgtcggggaagctgacgtcctttccatggctgctcgcctgtgttgc |
| | cacctggattctgcgcgggacgtccttctgctacgtcccttcggccctcaatccagcgga |
| | ccttccttcccgcggcctgctgccggctctgcggcctcttccgcgtcttcgccttcgccc |
| | tcagacgagtcggatctccctttgggccgcctccccgcctggagaattcgatatcagtgg |
| | tccaggctctagttttgactcaacaatatcaccagctgaagcctatagagtacgagccat |
| | agataaaataaaagattttatttagtctccagaaaaaggggggaatgaaagaccccacct |
| | gtaggtttggcaagctagcaataaaagagcccacaacccctcactcgggggccagtcctc |
| | cgattgactgagtcgcccggccgcttcgagcagacatgataagatacattgatgagtttg |
| | gacaaaccacaactagaatgcagtgaaaaaaatgctttatttgtgaaatttgtgatgcta |
| | ttgctttatttgtaaccattataagctgcaataaacaagttaacaacaacaattgcattc |
| | attttatgtttcaggttcagggggagatgtgggaggttttttaaagcaagtaaaacctct |
| | acaaatgtggtaaaatcgataaggatcgggtacccgtgtatccaataaaccctcttgcag |
| | ttgcatccgacttgtggtctcgctgttccttgggagggtctcctctgagtgattgactac |
| | ccgtcagcgggggtctttcacacatgcagcatgtatcaaaattaatttggttttttttct |
| | taagctgtgccttctagttgccagccatctgttgtttgcccctcccccgtgccttccttg |
| | accctggaaggtgccactcccactgtcctttcctaataaaatgaggaaattgcatcgcat |
| | tgtctgagtaggtgtcattctattctggggggtggggggggcaggacagcaagggggagg |
| | attgggaagacaatagcaggcatgctggggatgcggtgggctctatggagatcccgcggt |
| | acctcgcgaatgcatctagatccaatggcctttttggcccagacatgataagatacattg |
| | atgagtttggacaaaccacaactagaatgcagtgaaaaaaatgctttatttgtgaaattt |
| | gtgatgctattgctttatttgtaaccattataagctgcaataaacaagttgcggccgctt |
| | agccctcccacacataaccagagggcagcaattcacgaatcccaactgccgtcggctgtc |
| | catcactgtccttcactatggctttgatcccaggatgcagatcgagaagcacctgtcggc |
| | accgtccgcaggggctcaagatgcccctgttctcatttccgatcgcgacgatacaagtca |
| | ggttgccagctgccgcagcagcagcagtgcccagcaccacgagttctgcacaaggtcccc |
| | cagtaaaatgatatacattgacaccagtgaagatgcggccgtcgctagagagagctgcgc |
| | tggcgacgctgtagtcttcagagatggggatgctgttgattgtagccgttgctctttcaa |
| | tgagggtggattcttcttgagacaaaggcttggccatgcggccgccgctcggtgttcgag |
| | gccacacgcgtcaccttaatatgcgaagtggacctcggaccgcgccgccccgactgcatc |
| | tgcgtgttcgaattcgccaatgacaagacgctgggggggtttgtgtcatcatagaactaa |
| | agacatgcaaatatatttcttccggggggtaccggcctttttggccATTGGatcggatct |
| | ggccaaaaaggcccttaagtatttacattaaatggccatagtacttaaagttacattggc |
| | ttccttgaaataaacatggagtattcagaatgtgtcataaatatttctaattttaagata |
| | gtatctccattggctttctactttttcttttatttttttttgtcctctgtcttccatttg |
| | ttgttgttgttgtttgtttgtttgtttgttggttggttggttaatttttttttaaagatc |
| | ctacactatagttcaagctagactattagctactctgtaacccagggtgaccttgaagtc |
| | atgggtagcctgctgttttagccttcccacatctaagattacaggtatgagctatcattt |
| | ttggtatattgattgattgattgattgatgtgtgtgtgtgtgattgtgtttgtgtgtgtg |
| | aTtgtgTaTatgtgtgtatggTtgtgtgtgaTtgtgtgtatgtatgTTtgtgtgtgaTtg |
| | TgtgtgtgtgaTtgtgcatgtgtgtgtgtgtgaTtgtgtTtatgtgtatgaTtgtgtgtg |
| | tgtgtgtgtgtgtgtgtgtgtgtgtgtgtgtgtgtgtgttgtgTaTaTatatttatggta |
| | gtgagagGcaacgctccggctcaggtgtcaggttggtttttgagacagagtctttcactt |
| | agcttggaattcactggccgtcgttttacaacgtcgtgactgggaaaaccctggcgttac |
| | ccaacttaatcgccttgcagcacatccccctttcgccagctggcgtaatagcgaagaggc |
| | ccgcaccgatcgcccttcccaacagttgcgcagcctgaatggcgaatggcgcctgatgcg |
| | gtattttctccttacgcatctgtgcggtatttcacaccgcatatggtgcactctcagtac |
| | aatctgctctgatgccgcatagttaagccagccccgacacccgccaacacccgctgacgc |
| | gccctgacgggcttgtctgctcccggcatccgcttacagacaagctgtgaccgtctccgg |
| | gagctgcatgtgtcagaggttttcaccgtcatcaccgaaacgcgcgagacgaaagggcct |
| | cgtgatacgcctatttttataggttaatgtcatgataataatggtttcttagacgtcagg |
| | tggcacttttcggggaaatgtgcgcggaacccctatttgtttatttttctaaatacattc |
| | aaatatgtatccgctcatgagacaataaccctgataaatgcttcaataatattgaaaaag |
| | gaagagtatgagtattcaacatttccgtgtcgcccttattcccttttttgcggcattttg |
| | ccttcctgtttttgctcacccagaaacgctggtgaaagtaaaagatgctgaagatcagtt |
| | gggtgcacgagtgggttacatcgaactggatctcaacagcggtaagatccttgagagttt |
| | tcgccccgaagaacgttttccaatgatgagcacttttaaagttctgctatgtggcgcggt |
| | attatcccgtattgacgccgggcaagagcaactcggtcgccgcatacactattctcagaa |
| | tgacttggttgagtactcaccagtcacagaaaagcatcttacggatggcatgacagtaag |
| | agaattatgcagtgctgccataaccatgagtgataacactgcggccaacttacttctgac |
| | aacgatcggaggaccgaaggagctaaccgcttttttgcacaacatgggggatcatgtaac |
| | tcgccttgatcgttgggaaccggagctgaatgaagccataccaaacgacgagcgtgacac |
| | cacgatgcctgtagcaatggcaacaacgttgcgcaaactattaactggcgaactacttac |
| | tctagcttcccggcaacaattaatagactggatggaggcggataaagttgcaggaccact |
| | tctgcgctcggcccttccggctggctggtttattgctgataaatctggagccggtgagcg |
| | tgggtctcgcggtatcattgcagcactggggccagatggtaagccccccgtatcgtagtt |
| | atctacacgacggggagtcaggcaactatggatgaacgaaatagacagatcgctgagata |
| | ggtgcctcactgattaagcattggtaactgtcagaccaagtttactcatatatactttag |
| | attgatttaaaacttcatttttaatttaaaaggatctaggtgaagatcctttttgataat |
| | ctcatgaccaaaatcccttaacgtgagttttcgttccactgagcgtcagaccccgtagaa |
| | aagatcaaaggatcttcttgagatcctttttttctgcgcgtaatctgctgcttgcaaaca |
| | aaaaaaccaccgctaccagcggtggtttgtttgccggatcaagagctaccaactcttttt |
| | ccgaaggtaactggcttcagcagagcgcagataccaaatactgtccttctagtgtagccg |
| | tagttaggccaccacttcaagaactctgtagcaccgcctacatacctcgctctgctaatc |
| | ctgttaccagtggctgctgccagtggcgataagtcgtgtcttaccgggttggactcaaga |
| | cgatagttaccggataaggcgcagcggtcgggctgaacggggggttcgtgcacacagccc |
| | agcttggagcgaacgacctacaccgaactgagatacctacagcgtgagctatgagaaagc |
| | gccacgcttcccgaagggagaaaggcggacaggtatccggtaagcggcagggtcggaaca |
| | ggagagcgcacgagggagcttccagggggaaacgcctggtatctttatagtcctgtcggg |
| | tttcgccacctctgacttgagcgtcgatttttgtgatgctcgtcaggggggggagcctat |
| | ggaaaaacgccagcaacgcggcctttttacggttcctggccttttgctggccttttgctc |
| | acatgttctttcctgcgttatcccctgattctgtggataaccgtattaccgcctttgagt |
| | gagctgataccgctcgccgcagccgaacgaccgagcgcagcgagtcagtgagcgaggaag |
| | cggaagagcgcccaatacgcaaaccgcctctccccgcgcgttggccgattcattaatgca |
| | gctggcacgacaggtttcccgactggaaagcgggcagtgagcgcaacgcaattaatgtga |
| | gttagctcactcattaggcaccccaggctttacactttatgcttccggctcgtatgttgt |
| | gtggaattgtgagcggataacaatttcacacaggaaacagctatgaccatgattacgcc |
| | (SEQ ID NO: 310) |
| SB06298 | aagcttgaattcgagcttgcatgcctgcaggtcgttacataacttacggtaaatggcccg |
| | cctggctgaccgcccaacgacccccgcccattgacgtcaataatgacgtatgttcccata |
| | gtaacgccaatagggactttccattgacgtcaatgggtggagtatttacggtaaactgcc |
| | cacttggcagtacatcaagtgtatcatatgccaagtacgccccctattgacgtcaatgac |
| | ggtaaatggcccgcctggcattatgcccagtacatgaccttatgggactttcctacttgg |
| | cagtacatctacgtattagtcatcgctattaccatggtgatgcggttttggcagtacatc |
| | aatgggcgtggatagcggtttgactcacggggatttccaagtctccaccccattgacgtc |
| | aatgggagtttgttttggcaccaaaatcaacgggactttccaaaatgtcgtaacaactcc |
| | gccccattgacgcaaatgggggtaggcgtgtacggtgggaggtctatataagcagagctc |
| | aataaaagagcccacaacccctcactcggcgcgccagtcctccgattgactgagtcgccc |
| | gggtacccgtgtatccaataaaccctcttgcagttgcatccgacttgtggtctcgctgtt |
| | ccttgggagggtctcctctgagtgattgactacccgtcagcgggggtctttcatttgggg |
| | gctcgtccgagatcgggagacccctgcccagggaccaccgacccaccaccgggaggtaag |
| | ctggccagcaacttatctgtgtctgtccgattgtctagtgtctatgactgattttatgcg |
| | cctgcgtcggtactagttagctaactagctctgtatctggcggacccgtggtggaactga |
| | cgagttcggaacacccggccgcaaccctgggagacgtcccagggacttcgggggccgttt |
| | ttgtggcccgacctgagtcctaaaatcccgatcgtttaggactctttggtgcacccccct |
| | tagaggagggatatgtggttctggtaggagacgagaacctaaaacagttcccgcctccgt |
| | ctgaatttttgctttcggtttgggaccgaagccgcgccgcgcgtcttgtctgctgcagca |
| | tcgttctgtgttgtctctgtctgactgtgtttctgtatttgtctgaaaatatgggccccc |
| | cctcgaggtaacgccattttgcaaggcatggaaaaataccaaaccaagaatagagaagtt |
| | cagatcaagggcgggtacatgaaaatagctaacgttgggccaaacaggatatctgcggtg |
| | agcagtttcggccccggcccggggccaagaacagatggtcaccgcagtttcggccccggc |
| | ccgaggccaagaacagatggtccccagatatggcccaaccctcagcagtttcttaagacc |
| | catcagatgtttccaggctcccccaaggacctgaaatgaccctgcgccttatttgaatta |
| | accaatcagcctgcttctcgcttctgttcgcgcgcttctgcttcccgagctctataaaag |
| | agctcacaacccctcactcggcgcgccagtcctccgacagactgagtcgcccgggGCCGC |
| | CACCATGCTGCTGCTGGTCACATCTCTGCTGCTGTGCGAGCTGCCCCATCCTGCCTTTCT |
| | GCTGATCCCTCACATGGAAGTGCAGCTGGTGGAATCTGGCGGAGGACTGGTTCAACCTGG |
| | CGGCTCTCTGAGACTGTCTTGTGCCGCCAGCGGCTTCACCTTCAACAAGAACGCCATGAA |
| | CTGGGTCCGACAGGCCCCTGGCAAAGGCCTTGAATGGGTCGGACGGATCCGGAACAAGAC |
| | CAACAACTACGCCACCTACTACGCCGACAGCGTGAAGGCCAGGTTCACCATCTCCAGAGA |
| | TGACAGCAAGAACAGCCTGTACCTGCAGATGAACTCCCTGAAAACCGAGGACACCGCCGT |
| | GTACTATTGCGTGGCCGGCAATAGCTTTGCCTACTGGGGACAGGGCACCCTGGTTACAGT |
| | TTCTGCTGGCGGCGGAGGAAGCGGAGGCGGAGGATCCGGTGGTGGTGGATCTGACATCGT |
| | GATGACACAGAGCCCCGATAGCCTGGCCGTGTCTCTGGGAGAAAGAGCCACCATCAACTG |
| | CAAGAGCAGCCAGAGCCTGCTGTACTCCAGCAACCAGAAGAACTACCTGGCCTGGTATCA |
| | GCAAAAGCCCGGCCAGCCTCCTAAGCTGCTGATCTATTGGGCCAGCTCCAGAGAAAGCGG |
| | CGTGCCCGATAGATTTTCTGGCTCTGGCAGCGGCACCGACTTCACCCTGACAATTTCTAG |
| | CCTGCAAGCCGAGGACGTGGCCGTGTATTACTGCCAGCAGTACTACAACTACCCTCTGAC |
| | CTTCGGCCAGGGCACCAAGCTGGAAATCAAATCTGGCGCCCTGAGCAACAGCATCATGTA |
| | CTTCAGCCACTTCGTGCCCGTGTTTCTGCCCGCCAAGCCTACAACAACCCCTGCTCCTAG |
| | ACCTCCTACACCAGCTCCTACAATCGCCAGCCAGCCTCTGTCTCTGAGGCCAGAAGCTTG |
| | TAGACCTGCTGCAGGCGGAGCCGTGCATACAAGAGGACTGGATTTCGCCTGCGACATCTA |
| | CATCTGGGCCCCTCTGGCTGGAACATGTGGTGTCCTGCTGCTGAGCCTGGTCATCACCCT |
| | GTACTGCAACCACCGGCGGAGCAAGAGAAGCAGACTGCTGCACAGCGACTACATGAACAT |
| | GACCCCTAGACGGCCCGGACCTACCAGAAAGCACTACCAGCCTTACGCTCCTCCTAGAGA |
| | CTTCGCCGCCTACCGGTCCAGAGTGAAGTTCAGCAGGAGCGCAGACGCCCCCGCGTACAA |
| | GCAGGGCCAGAACCAGCTCTATAACGAGCTCAATCTAGGACGAAGAGAGGAGTACGATGT |
| | TTTGGACAAGAGACGTGGCCGGGACCCTGAGATGGGGGGAAAGCCGAGAAGGAAGAACCC |
| | TCAGGAAGGCCTGTACAATGAACTGCAGAAAGATAAGATGGCGGAGGCCTACAGTGAGAT |
| | TGGGATGAAAGGCGAGCGCCGGAGGGGCAAGGGGCACGATGGCCTTTACCAGGGTCTCAG |
| | TACAGCCACCAAGGACACCTACGACGCCCTTCACATGCAGGCCCTGCCCCCTCGCCAGTG |
| | TACCAACTACGCCCTGCTGAAACTGGCCGGCGACGTGGAATCTAATCCTGGACCTGGATC |
| | TGGCGAGGGACGCGGGAGTCTACTGACGTGTGGAGACGTGGAGGAAAACCCTGGACCTAT |
| | GGACTGGACCTGGATCCTGTTTCTGGTGGCCGCTGCCACAAGAGTGCACAGCAATTGGGT |
| | CAACGTGATCAGCGACCTGAAGAAGATCGAGGACCTGATCCAGAGCATGCACATCGACGC |
| | CACACTGTACACCGAGAGCGACGTGCACCCTAGCTGTAAAGTGACCGCCATGAAGTGCTT |
| | TCTGCTGGAACTGCAAGTGATCAGCCTGGAAAGCGGCGACGCCAGCATCCACGACACCGT |
| | GGAAAACCTGATCATCCTGGCCAACAACAGCCTGAGCAGCAACGGCAATGTGACCGAGTC |
| | CGGCTGCAAAGAGTGCGAGGAACTGGAAGAGAAGAATATCAAAGAGTTCCTGCAGAGCTT |
| | CGTGCACATCGTGCAGATGTTCATCAACACAAGCTCTGGCGGCGGAGGATCTGGCGGAGG |
| | TGGAAGCGGAGTTACACCCGAGCCTATCTTCAGCCTGATCGGAGGCGGTAGCGGAGGCGG |
| | AGGAAGTGGTGGCGGATCTCTGCAACTGCTGCCTAGCTGGGCCATCACACTGATCTCCGT |
| | GAACGGCATCTTCGTGATCTGCTGCCTGACCTACTGCTTCGCCCCTAGATGCAGAGAGCG |
| | GCGGAGAAACGAACGGCTGAGAAGAGAATCTGTGCGGCCCGTTtaaggatccggattagt |
| | ccaatttgttaaagacaggatgggctgcaggaattccgataatcaacctctggattacaa |
| | aatttgtgaaagattgactggtattcttaactatgttgctccttttacgctatgtggata |
| | cgctgctttaatgcctttgtatcatgctattgcttcccgtatggctttcattttctcctc |
| | cttgtataaatcctggttgctgtctctttatgaggagttgtggcccgttgtcaggcaacg |
| | tggcgtggtgtgcactgtgtttgctgacgcaacccccactggttggggcattgccaccac |
| | ctgtcagctcctttccgggactttcgctttccccctccctattgccacggcggaactcat |
| | cgccgcctgccttgcccgctgctggacaggggctcggctgttgggcactgacaattccgt |
| | ggtgttgtcggggaagctgacgtcctttccatggctgctcgcctgtgttgccacctggat |
| | tctgcgcgggacgtccttctgctacgtcccttcggccctcaatccagcggaccttccttc |
| | ccgcggcctgctgccggctctgcggcctcttccgcgtcttcgccttcgccctcagacgag |
| | tcggatctccctttgggccgcctccccgcctggagaattcgatatcagtggtccaggctc |
| | tagttttgactcaacaatatcaccagctgaagcctatagagtacgagccatagataaaat |
| | aaaagattttatttagtctccagaaaaaggggggaatgaaagaccccacctgtaggtttg |
| | gcaagctagcaataaaagagcccacaacccctcactcggggcgccagtcctccgattgac |
| | tgagtcgcccggccgcttcgagcagacatgataagatacattgatgagtttggacaaacc |
| | acaactagaatgcagtgaaaaaaatgctttatttgtgaaatttgtgatgctattgcttta |
| | tttgtaaccattataagctgcaataaacaagttaacaacaacaattgcattcattttatg |
| | tttcaggttcagggggagatgtgggaggttttttaaagcaagtaaaacctctacaaatgt |
| | ggtaaaatcgataaggatcgggtacccgtgtatccaataaaccctcttgcagttgcatcc |
| | gacttgtggtctcgctgttccttgggagggtctcctctgagtgattgactacccgtcagc |
| | gggggtctttcacacatgcagcatgtatcaaaattaatttggttttttttcttaagctgt |
| | gccttctagttgccagccatctgttgtttgcccctcccccgtgccttccttgaccctgga |
| | aggtgccactcccactgtcctttcctaataaaatgaggaaattgcatcgcattgtctgag |
| | taggtgtcattctattctggggggtggggggggcaggacagcaagggggaggattgggaa |
| | gacaatagcaggcatgctggggatgcggtgggctctatggagatcccgcggtacctcgcg |
| | aatgcatctagatccaatggcctttttggcccagacatgataagatacattgatgagttt |
| | ggacaaaccacaactagaatgcagtgaaaaaaatgctttatttgtgaaatttgtgatgct |
| | attgctttatttgtaaccattataagctgcaataaacaagttgcggccgcttagccctcc |
| | cacacataaccagagggcagcaattcacgaatcccaactgccgtcggctgtccatcactg |
| | tccttcactatggctttgatcccaggatgcagatcgagaagcacctgtcggcaccgtccg |
| | caggggctcaagatgcccctgttctcatttccgatcgcgacgatacaagtcaggttgcca |
| | gctgccgcagcagcagcagtgcccagcaccacgagttctgcacaaggtcccccagtaaaa |
| | tgatatacattgacaccagtgaagatgcggccgtcgctagagagagctgcgctggcgacg |
| | ctgtagtcttcagagatggggatgctgttgattgtagccgttgctctttcaatgagggtg |
| | gattcttcttgagacaaaggcttggccatgcggccgccgctcggtgttcgaggccacacg |
| | cgtcaccttaatatgcgaagtggacctcggaccgcgccgccccgactgcatctgcgtgtt |
| | cgaattcgccaatgacaagacgctgggcggggtttgtgtcatcatagaactaaagacatg |
| | caaatatatttcttccggggggtaccggcctttttggccATTGGatcggatctggccaaa |
| | aaggcccttaagtatttacattaaatggccatagtacttaaagttacattggcttccttg |
| | aaataaacatggagtattcagaatgtgtcataaatatttctaattttaagatagtatctc |
| | cattggctttctactttttcttttatttttttttgtcctctgtcttccatttgttgttgt |
| | tgttgtttgtttgtttgtttgttggttggttggttaatttttttttaaagatcctacact |
| | atagttcaagctagactattagctactctgtaacccagggtgaccttgaagtcatgggta |
| | gcctgctgttttagccttcccacatctaagattacaggtatgagctatcatttttggtat |
| | attgattgattgattgattgatgtgtgtgtgtgtgattgtgtttgtgtgtgtgaTtgtgT |
| | aTatgtgtgtatggTtgtgtgtgaTtgtgtgtatgtatgTTtgtgtgtgaTtgTgtgtgt |
| | gtgaTtgtgcatgtgtgtgtgtgtgaTtgtgtTtatgtgtatgaTtgtgtgtgtgtgtgt |
| | gtgtgtgtgtgtgtgtgtgtgtgtgtgtgtgttgtgTaTaTatatttatggtagtgagag |
| | Gcaacgctccggctcaggtgtcaggttggtttttgagacagagtctttcacttagcttgg |
| | aattcactggccgtcgttttacaacgtcgtgactgggaaaaccctggcgttacccaactt |
| | aatcgccttgcagcacatccccctttcgccagctggcgtaatagcgaagaggcccgcacc |
| | gatcgcccttcccaacagttgcgcagcctgaatggcgaatggcgcctgatgcggtatttt |
| | ctccttacgcatctgtgcggtatttcacaccgcatatggtgcactctcagtacaatctgc |
| | tctgatgccgcatagttaagccagccccgacacccgccaacacccgctgacgcgccctga |
| | cgggcttgtctgctcccggcatccgcttacagacaagctgtgaccgtctccgggagctgc |
| | atgtgtcagaggttttcaccgtcatcaccgaaacgcgcgagacgaaagggcctcgtgata |
| | cgcctatttttataggttaatgtcatgataataatggtttcttagacgtcaggtggcact |
| | tttcggggaaatgtgcgcggaacccctatttgtttatttttctaaatacattcaaatatg |
| | tatccgctcatgagacaataaccctgataaatgcttcaataatattgaaaaaggaagagt |
| | atgagtattcaacatttccgtgtcgcccttattcccttttttgcggcattttgccttcct |
| | gtttttgctcacccagaaacgctggtgaaagtaaaagatgctgaagatcagttgggtgca |
| | cgagtgggttacatcgaactggatctcaacagcggtaagatccttgagagttttcgcccc |
| | gaagaacgttttccaatgatgagcacttttaaagttctgctatgtggcgcggtattatcc |
| | cgtattgacgccgggcaagagcaactcggtcgccgcatacactattctcagaatgacttg |
| | gttgagtactcaccagtcacagaaaagcatcttacggatggcatgacagtaagagaatta |
| | tgcagtgctgccataaccatgagtgataacactgcggccaacttacttctgacaacgatc |
| | ggaggaccgaaggagctaaccgcttttttgcacaacatgggggatcatgtaactcgcctt |
| | gatcgttgggaaccggagctgaatgaagccataccaaacgacgagcgtgacaccacgatg |
| | cctgtagcaatggcaacaacgttgcgcaaactattaactggcgaactacttactctagct |
| | tcccggcaacaattaatagactggatggaggcggataaagttgcaggaccacttctgcgc |
| | tcggcccttccggctggctggtttattgctgataaatctggagccggtgagcgtgggtct |
| | cgcggtatcattgcagcactggggccagatggtaagccctcccgtatcgtagttatctac |
| | acgacggggagtcaggcaactatggatgaacgaaatagacagatcgctgagataggtgcc |
| | tcactgattaagcattggtaactgtcagaccaagtttactcatatatactttagattgat |
| | ttaaaacttcatttttaatttaaaaggatctaggtgaagatcctttttgataatctcatg |
| | accaaaatcccttaacgtgagttttcgttccactgagcgtcagaccccgtagaaaagatc |
| | aaaggatcttcttgagatcctttttttctgcgcgtaatctgctgcttgcaaacaaaaaaa |
| | ccaccgctaccagcggtggtttgtttgccggatcaagagctaccaactctttttccgaag |
| | gtaactggcttcagcagagcgcagataccaaatactgtccttctagtgtagccgtagtta |
| | ggccaccacttcaagaactctgtagcaccgcctacatacctcgctctgctaatcctgtta |
| | ccagtggctgctgccagtggcgataagtcgtgtcttaccgggttggactcaagacgatag |
| | ttaccggataaggcgcagcggtcgggctgaacggggggttcgtgcacacagcccagcttg |
| | gagcgaacgacctacaccgaactgagatacctacagcgtgagctatgagaaagcgccacg |
| | cttcccgaagggagaaaggcggacaggtatccggtaagcggcagggtcggaacaggagag |
| | cgcacgagggagcttccagggggaaacgcctggtatctttatagtcctgtcgggtttcgc |
| | cacctctgacttgagcgtcgatttttgtgatgctcgtcaggggggcggagcctatggaaa |
| | aacgccagcaacgcggcctttttacggttcctggccttttgctggccttttgctcacatg |
| | ttctttcctgcgttatcccctgattctgtggataaccgtattaccgcctttgagtgagct |
| | gataccgctcgccgcagccgaacgaccgagcgcagcgagtcagtgagcgaggaagcggaa |
| | gagcgcccaatacgcaaaccgcctctccccgcgcgttggccgattcattaatgcagctgg |
| | cacgacaggtttcccgactggaaagcgggcagtgagcgcaacgcaattaatgtgagttag |
| | ctcactcattaggcaccccaggctttacactttatgcttccggctcgtatgttgtgtgga |
| | attgtgagcggataacaatttcacacaggaaacagctatgaccatgattacgcc |
| | (SEQ ID NO: 311) |
| SB06254 | aagctttgctcttaggagtttcctaatacatcccaaactcaaatatataaagcatttgac |
| | ttgttctatgccctagggggcggggggaagctaagccagctttttttaacatttaaaatg |
| | ttaattccattttaaatgcacagatgtttttatttcataagggtttcaatgtgcatgaat |
| | gctgcaatattcctgttaccaaagctagtataaataaaaatagataaacgtggaaattac |
| | ttagagtttctgtcattaacgtttccttcctcagttgacaacataaatgcgctgctgaga |
| | agccagtttgcatctgtcaggatcaatttcccattatgccagtcatattaattactagtc |
| | aattagttgatttttatttttgacatatacatgtgaaagaccccacctgtaggtttggca |
| | agctagcttaagtaacgccattttgcaaggcatggaaaaatacataactgagaatagaaa |
| | agttcagatcaaggtcaggaacagatggaacagctgaatatgggccaaacaggatatctg |
| | tggtaagcagttcctgccccggctcagggccaagaacagatggaacagctgaatatgggc |
| | caaacaggatatctgtggtaagcagttcctgccccggctcagggccaagaacagatggtc |
| | cccagatgcggtccagccctcagcagtttctagagaaccatcagatgtttccagggtgcc |
| | ccaaggacctgaaatgaccctgtgccttatttgaactaaccaatcagttcgcttctcgct |
| | tctgttcgcgcgcttctgctccccgagctcaataaaagagcccacaacccctcactcggc |
| | gcgccagtcctccgattgactgagtcgcccgggtacccgtgtatccaataaaccctcttg |
| | cagttgcatccgacttgtggtctcgctgttccttgggagggtctcctctgagtgattgac |
| | tacccgtcagcgggggtctttcatttgggggctcgtccgagatcgggagacccctgccca |
| | gggaccaccgacccaccaccgggaggtaagctggccagcaacttatctgtgtctgtccga |
| | ttgtctagtgtctatgactgattttatgcgcctgcgtcggtactagttagctaactagct |
| | ctgtatctggcggacccgtggtggaactgacgagttcggaacacccggccgcaaccctgg |
| | gagacgtcccagggacttcgggggccgtttttgtggcccgacctgagtcctaaaatcccg |
| | atcgtttaggactctttggtgcaccccccttagaggagggatatgtggttctggtaggag |
| | acgagaacctaaaacagttcccgcctccgtctgaatttttgctttcggtttgggaccgaa |
| | gccgcgccgcgcgtcttgtctgctgcagcatcgttctgtgttgtctctgtctgactgtgt |
| | ttctgtatttgtctgaaaatatggatcttatatggggcacccccgccccttgtaaacttc |
| | cctgaccctgacatgacaagagttactaacagcccctctctccaagctcacttacaggct |
| | ctctacttagtccagcacgaagtctggagacctctggcggcagcctaccaagaacaactg |
| | gaccgaccggtggtacctcacccttaccgagtcggcgacacagtgtgggtccgccgacac |
| | cagactaagaacctagaacctcgctggaaaggaccttacacagtcctgctgaccaccccc |
| | accgccctcaaagtagacggcatcgcagcttggatacacgccgcccacgtgaaggctgcc |
| | gaccccgggggtggaccatcctctagactgccggatccGCCGCCACCATGGACTGGACCT |
| | GGATCCTGTTTCTGGTGGCCGCTGCCACAAGAGTGCACAGCAATTGGGTCAACGTGATCA |
| | GCGACCTGAAGAAGATCGAGGACCTGATCCAGAGCATGCACATCGACGCCACACTGTACA |
| | CCGAGAGCGACGTGCACCCTAGCTGTAAAGTGACCGCCATGAAGTGCTTTCTGCTGGAAC |
| | TGCAAGTGATCAGCCTGGAAAGCGGCGACGCCAGCATCCACGACACCGTGGAAAACCTGA |
| | TCATCCTGGCCAACAACAGCCTGAGCAGCAACGGCAATGTGACCGAGTCCGGCTGCAAAG |
| | AGTGCGAGGAACTGGAAGAGAAGAATATCAAAGAGTTCCTGCAGAGCTTCGTGCACATCG |
| | TGCAGATGTTCATCAACACAAGCTCTGGCGGCGGAGGATCTGGCGGAGGTGGAAGCGGAG |
| | TTACACCCGAGCCTATCTTCAGCCTGATCGGAGGCGGTAGCGGAGGCGGAGGAAGTGGTG |
| | GCGGATCTCTGCAACTGCTGCCTAGCTGGGCCATCACACTGATCTCCGTGAACGGCATCT |
| | TCGTGATCTGCTGCCTGACCTACTGCTTCGCCCCTAGATGCAGAGAGCGGCGGAGAAACG |
| | AACGGCTGAGAAGAGAATCTGTGCGGCCCGTTGGTAGCGGCCAGTGTACCAACTACGCCC |
| | TGCTGAAACTGGCCGGCGACGTGGAATCTAATCCTGGACCTGGATCTGGCGAGGGACGCG |
| | GGAGTCTACTGACGTGTGGAGACGTGGAGGAAAACCCTGGACCTATGCTGCTGCTGGTCA |
| | CATCTCTGCTGCTGTGCGAGCTGCCCCATCCTGCCTTTCTGCTGATCCCTCACATGGACA |
| | TCGTGATGACACAGAGCCCCGATAGCCTGGCCGTGTCTCTGGGAGAAAGAGCCACCATCA |
| | ACTGCAAGAGCAGCCAGAGCCTGCTGTACTCCAGCAACCAGAAGAACTACCTGGCCTGGT |
| | ATCAGCAAAAGCCCGGCCAGCCTCCTAAGCTGCTGATCTATTGGGCCAGCTCCAGAGAAA |
| | GCGGCGTGCCCGATAGATTTTCTGGCTCTGGCAGCGGCACCGACTTCACCCTGACAATTT |
| | CTAGCCTGCAAGCCGAGGACGTGGCCGTGTACTACTGCCAGCAGTACTACAACTACCCTC |
| | TGACCTTCGGCCAGGGCACCAAGCTGGAAATCAAAGGCGGCGGAGGATCTGGCGGAGGTG |
| | GAAGTGGCGGAGGCGGATCTGAAGTGCAGCTGGTTGAATCAGGTGGCGGCCTGGTTCAAC |
| | CTGGCGGATCTCTGAGACTGAGCTGTGCCGCCAGCGGCTTCACCTTCAACAAGAACGCCA |
| | TGAACTGGGTCCGACAGGCCCCTGGCAAAGGCCTTGAATGGGTCGGACGGATCCGGAACA |
| | AGACCAACAACTACGCCACCTACTACGCCGACAGCGTGAAGGCCAGATTCACCATCAGCC |
| | GGGACGACAGCAAGAACAGCCTGTACCTGCAGATGAACTCCCTGAAAACCGAGGACACCG |
| | CCGTGTATTATTGCGTGGCCGGCAACAGCTTTGCCTACTGGGGACAGGGAACCCTGGTCA |
| | CCGTGTCTGCCACAACAACCCCTGCTCCTAGACCTCCTACACCAGCTCCTACAATCGCCC |
| | TGCAGCCTCTGTCTCTGAGGCCAGAAGCTTGTAGACCAGCTGCTGGCGGAGCCGTGCATA |
| | CAAGAGGACTGGACTTCGCCTGTGATGTGGCCGCCATTCTCGGACTGGGACTTGTTCTGG |
| | GACTGCTGGGACCTCTGGCCATTCTGCTGGCTCTGTATCTGCTGCGGAGGGACCAAAGAC |
| | TGCCTCCTGATGCTCACAAGCCTCCAGGCGGAGGCAGCTTCAGAACCCCTATCCAAGAGG |
| | AACAGGCCGACGCTCACAGCACCCTGGCCAAGATTAGAGTGAAGTTCAGCAGAAGCGCCG |
| | ACGCACCCGCCTATAAGCAGGGACAGAACCAGCTGTACAACGAGCTGAACCTGGGGAGAA |
| | GAGAAGAGTACGACGTGCTGGACAAGCGGAGAGGCAGAGATCCTGAGATGGGCGGCAAGC |
| | CCAGACGGAAGAATCCTCAAGAGGGCCTGTATAATGAGCTGCAGAAAGACAAGATGGCCG |
| | AGGCCTACAGCGAGATCGGAATGAAGGGCGAGCGCAGAAGAGGCAAGGGACACGATGGAC |
| | TGTACCAGGGCCTGAGCACCGCCACCAAGGATACCTATGATGCCCTGCACATGCAGGCCC |
| | TGCCTCCAAGAtaaagatctagatccggattagtccaatttgttaaagacaggatatcag |
| | tggtccaggctctagttttgactcaacaatatcaccagctgaagcctatagagtacgagc |
| | catagataaaataaaagattttatttagtctccagaaaaaggggggaatgaaagacccca |
| | cctgtaggtttggcaagctagcttaagtaacgccattttgcaaggcatggaaaaatacat |
| | aactgagaatagagaagttcagatcaaggtcaggaacagatggaacagctgaatatgggc |
| | caaacaggatatctgtggtaagcagttcctgccccggctcagggccaagaacagatggaa |
| | cagctgaatatgggccaaacaggatatctgtggtaagcagttcctgccccggctcagggc |
| | caagaacagatggtccccagatgcggtccagccctcagcagtttctagagaaccatcaga |
| | tgtttccagggtgccccaaggacctgaaatgaccctgtgccttatttgaactaaccaatc |
| | agttcgcttctcgcttctgttcgcgcgcttctgctccccgagctcaataaaagagcccac |
| | aacccctcactcggggcgccagtcctccgattgactgagtcgcccgggtacccgtgtatc |
| | caataaaccctcttgcagttgcatccgacttgtggtctcgctgttccttgggagggtctc |
| | ctctgagtgattgactacccgtcagcgggggtctttcacatgcagcatgtatcaaaatta |
| | atttggttttttttcttaagtatttacattaaatggccatagtacttaaagttacattgg |
| | cttccttgaaataaacatggagtattcagaatgtgtcataaatatttctaattttaagat |
| | agtatctccattggctttctactttttcttttatttttttttgtcctctgtcttccattt |
| | gttgttgttgttgtttgtttgtttgtttgttggttggttggttaatttttttttaaagat |
| | cctacactatagttcaagctagactattagctactctgtaacccagggtgaccttgaagt |
| | catgggtagcctgctgttttagccttcccacatctaagattacaggtatgagctatcatt |
| | tttggtatattgattgattgattgattgatgtgtgtgtgtgtgattgtgtttgtgtgtgt |
| | gaTtgtgTaTatgtgtgtatggTtgtgtgtgaTtgtgtgtatgtatgTTtgtgtgtgaTt |
| | gTgtgtgtgtgaTtgtgcatgtgtgtgtgtgtgaTtgtgtTtatgtgtatgaTtgtgtgt |
| | gtgtgtgtgtgtgtgtgtgtgtgtgtgtgtgtgtgtgtgttgtgTaTaTatatttatggt |
| | agtgagagGcaacgctccggctcaggtgtcaggttggtttttgagacagagtctttcact |
| | tagcttggaattaattcactggccgtcgttttacaacgtcgtgactgggaaaaccctggc |
| | gttacccaacttaatcgccttgcagcacatccccctttcgccagctggcgtaatagcgaa |
| | gaggcccgcaccgatcgcccttcccaacagttgcgcagcctgaatggcgaatggcgcctg |
| | atgcggtattttctccttacgcatctgtgcggtatttcacaccgcatatggtgcactctc |
| | agtacaatctgctctgatgccgcatagttaagccagccccgacacccgccaacacccgct |
| | gacgcgccctgacgggcttgtctgctcccggcatccgcttacagacaagctgtgaccgtc |
| | tccgggagctgcatgtgtcagaggttttcaccgtcatcaccgaaacgcgcgagacgaaag |
| | ggcctcgtgatacgcctatttttataggttaatgtcatgataataatggtttcttagacg |
| | tcaggtggcacttttcggggaaatgtgcgcggaacccctatttgtttatttttctaaata |
| | cattcaaatatgtatccgctcatgagacaataaccctgataaatgcttcaataatattga |
| | aaaaggaagagtatgagtattcaacatttccgtgtcgcccttattcccttttttgcggca |
| | ttttgccttcctgtttttgctcacccagaaacgctggtgaaagtaaaagatgctgaagat |
| | cagttgggtgcacgagtgggttacatcgaactggatctcaacagcggtaagatccttgag |
| | agttttcgccccgaagaacgttttccaatgatgagcacttttaaagttctgctatgtggc |
| | gcggtattatcccgtattgacgccgggcaagagcaactcggtcgccgcatacactattct |
| | cagaatgacttggttgagtactcaccagtcacagaaaagcatcttacggatggcatgaca |
| | gtaagagaattatgcagtgctgccataaccatgagtgataacactgcggccaacttactt |
| | ctgacaacgatcggaggaccgaaggagctaaccgcttttttgcacaacatgggggatcat |
| | gtaactcgccttgatcgttgggaaccggagctgaatgaagccataccaaacgacgagcgt |
| | gacaccacgatgcctgtagcaatggcaacaacgttgcgcaaactattaactggcgaacta |
| | cttactctagcttcccggcaacaattaatagactggatggaggcggataaagttgcagga |
| | ccacttctgcgctcggcccttccggctggctggtttattgctgataaatctggagccggt |
| | gagcgtgggtctcgcggtatcattgcagcactggggccagatggtaagccctcccgtatc |
| | gtagttatctacacgacggggagtcaggcaactatggatgaacgaaatagacagatcgct |
| | gagataggtgcctcactgattaagcattggtaactgtcagaccaagtttactcatatata |
| | ctttagattgatttaaaacttcatttttaatttaaaaggatctaggtgaagatccttttt |
| | gataatctcatgaccaaaatcccttaacgtgagttttcgttccactgagcgtcagacccc |
| | gtagaaaagatcaaaggatcttcttgagatcctttttttctgcgcgtaatctgctgcttg |
| | caaacaaaaaaaccaccgctaccagcggtggtttgtttgccggatcaagagctaccaact |
| | ctttttccgaaggtaactggcttcagcagagcgcagataccaaatactgtccttctagtg |
| | tagccgtagttaggccaccacttcaagaactctgtagcaccgcctacatacctcgctctg |
| | ctaatcctgttaccagtggctgctgccagtggcgataagtcgtgtcttaccgggttggac |
| | tcaagacgatagttaccggataaggcgcagcggtcgggctgaacggggggttcgtgcaca |
| | cagcccagcttggagcgaacgacctacaccgaactgagatacctacagcgtgagctatga |
| | gaaagcgccacgcttcccgaagggagaaaggcggacaggtatccggtaagcggcagggtc |
| | ggaacaggagagcgcacgagggagcttccagggggaaacgcctggtatctttatagtcct |
| | gtcgggtttcgccacctctgacttgagcgtcgatttttgtgatgctcgtcaggggggcgg |
| | agcctatggaaaaacgccagcaacgcggcctttttacggttcctggccttttgctggcct |
| | tttgctcacatgttctttcctgcgttatcccctgattctgtggataaccgtattaccgcc |
| | tttgagtgagctgataccgctcgccgcagccgaacgaccgagcgcagcgagtcagtgagc |
| | gaggaagcggaagagcgcccaatacgcaaaccgcctctccccgcgcgttggccgattcat |
| | taatgcagctggcacgacaggtttcccgactggaaagcgggcagtgagcgcaacgcaatt |
| | aatgtgagttagctcactcattaggcaccccaggctttacactttatgcttccggctcgt |
| | atgttgtgtggaattgtgagcggataacaatttcacacaggaaacagctatgaccatgat |
| | tacgcc (SEQ ID NO: 312) |
| SB06255 | aagctttgctcttaggagtttcctaatacatccc |
| | aaactcaaatatataaagcatttgacttgttctatgccctagggggcggggggaagctaa |
| | gccagctttttttaacatttaaaatgttaattccattttaaatgcacagatgtttttatt |
| | tcataagggtttcaatgtgcatgaatgctgcaatattcctgttaccaaagctagtataaa |
| | taaaaatagataaacgtggaaattacttagagtttctgtcattaacgtttccttcctcag |
| | ttgacaacataaatgcgctgctgagaagccagtttgcatctgtcaggatcaatttcccat |
| | tatgccagtcatattaattactagtcaattagttgatttttatttttgacatatacatgt |
| | gaaagaccccacctgtaggtttggcaagctagcttaagtaacgccattttgcaaggcatg |
| | gaaaaatacataactgagaatagaaaagttcagatcaaggtcaggaacagatggaacagc |
| | tgaatatgggccaaacaggatatctgtggtaagcagttcctgccccggctcagggccaag |
| | aacagatggaacagctgaatatgggccaaacaggatatctgtggtaagcagttcctgccc |
| | cggctcagggccaagaacagatggtccccagatgcggtccagccctcagcagtttctaga |
| | gaaccatcagatgtttccagggtgccccaaggacctgaaatgaccctgtgccttatttga |
| | actaaccaatcagttcgcttctcgcttctgttcgcgcgcttctgctccccgagctcaata |
| | aaagagcccacaacccctcactcggcgcgccagtcctccgattgactgagtcgcccgggt |
| | acccgtgtatccaataaaccctcttgcagttgcatccgacttgtggtctcgctgttcctt |
| | gggagggtctcctctgagtgattgactacccgtcagcgggggtctttcatttgggggctc |
| | gtccgagatcgggagacccctgcccagggaccaccgacccaccaccgggaggtaagctgg |
| | ccagcaacttatctgtgtctgtccgattgtctagtgtctatgactgattttatgcgcctg |
| | cgtcggtactagttagctaactagctctgtatctggcggacccgtggtggaactgacgag |
| | ttcggaacacccggccgcaaccctgggagacgtcccagggacttcgggggccgtttttgt |
| | ggcccgacctgagtcctaaaatcccgatcgtttaggactctttggtgcaccccccttaga |
| | ggagggatatgtggttctggtaggagacgagaacctaaaacagttcccgcctccgtctga |
| | atttttgctttcggtttgggaccgaagccgcgccgcgcgtcttgtctgctgcagcatcgt |
| | tctgtgttgtctctgtctgactgtgtttctgtatttgtctgaaaatatggatcttatatg |
| | gggcacccccgccccttgtaaacttccctgaccctgacatgacaagagttactaacagcc |
| | cctctctccaagctcacttacaggctctctacttagtccagcacgaagtctggagacctc |
| | tggcggcagcctaccaagaacaactggaccgaccggtggtacctcacccttaccgagtcg |
| | gcgacacagtgtgggtccgccgacaccagactaagaacctagaacctcgctggaaaggac |
| | cttacacagtcctgctgaccacccccaccgccctcaaagtagacggcatcgcagcttgga |
| | tacacgccgcccacgtgaaggctgccgaccccgggggtggaccatcctctagactgccgg |
| | atccGCCGCCACCATGGACTGGACCTGGATCCTGTTTCTGGTGGCCGCTGCCACAAGAGT |
| | GCACAGCAATTGGGTCAACGTGATCAGCGACCTGAAGAAGATCGAGGACCTGATCCAGAG |
| | CATGCACATCGACGCCACACTGTACACCGAGAGCGACGTGCACCCTAGCTGTAAAGTGAC |
| | CGCCATGAAGTGCTTTCTGCTGGAACTGCAAGTGATCAGCCTGGAAAGCGGCGACGCCAG |
| | CATCCACGACACCGTGGAAAACCTGATCATCCTGGCCAACAACAGCCTGAGCAGCAACGG |
| | CAATGTGACCGAGTCCGGCTGCAAAGAGTGCGAGGAACTGGAAGAGAAGAATATCAAAGA |
| | GTTCCTGCAGAGCTTCGTGCACATCGTGCAGATGTTCATCAACACAAGCTCTGGCGGCGG |
| | AGGATCTGGCGGAGGTGGAAGCGGAGTTACACCCGAGCCTATCTTCAGCCTGATCGGAGG |
| | CGGTAGCGGAGGCGGAGGAAGTGGTGGCGGATCTCTGCAACTGCTGCCTAGCTGGGCCAT |
| | CACACTGATCTCCGTGAACGGCATCTTCGTGATCTGCTGCCTGACCTACTGCTTCGCCCC |
| | TAGATGCAGAGAGCGGCGGAGAAACGAACGGCTGAGAAGAGAATCTGTGCGGCCCGTTGG |
| | TAGCGGCCAGTGTACCAACTACGCCCTGCTGAAACTGGCCGGCGACGTGGAATCTAATCC |
| | TGGACCTGGATCTGGCGAGGGACGCGGGAGTCTACTGACGTGTGGAGACGTGGAGGAAAA |
| | CCCTGGACCTATGCTGCTGCTGGTCACATCTCTGCTGCTGTGCGAGCTGCCCCATCCTGC |
| | CTTTCTGCTGATCCCTCACATGGAAGTGCAGCTGGTGGAATCTGGCGGAGGACTGGTTCA |
| | ACCTGGCGGCTCTCTGAGACTGTCTTGTGCCGCCAGCGGCTTCACCTTCAACAAGAACGC |
| | CATGAACTGGGTCCGACAGGCCCCTGGCAAAGGCCTTGAATGGGTCGGACGGATCCGGAA |
| | CAAGACCAACAACTACGCCACCTACTACGCCGACAGCGTGAAGGCCAGGTTCACCATCTC |
| | CAGAGATGACAGCAAGAACAGCCTGTACCTGCAGATGAACTCCCTGAAAACCGAGGACAC |
| | CGCCGTGTACTATTGCGTGGCCGGCAATAGCTTTGCCTACTGGGGACAGGGCACCCTGGT |
| | TACAGTTTCTGCTGGCGGCGGAGGAAGCGGAGGCGGAGGATCCGGTGGTGGTGGATCTGA |
| | CATCGTGATGACACAGAGCCCCGATAGCCTGGCCGTGTCTCTGGGAGAAAGAGCCACCAT |
| | CAACTGCAAGAGCAGCCAGAGCCTGCTGTACTCCAGCAACCAGAAGAACTACCTGGCCTG |
| | GTATCAGCAAAAGCCCGGCCAGCCTCCTAAGCTGCTGATCTATTGGGCCAGCTCCAGAGA |
| | AAGCGGCGTGCCCGATAGATTTTCTGGCTCTGGCAGCGGCACCGACTTCACCCTGACAAT |
| | TTCTAGCCTGCAAGCCGAGGACGTGGCCGTGTATTACTGCCAGCAGTACTACAACTACCC |
| | TCTGACCTTCGGCCAGGGCACCAAGCTGGAAATCAAATCTGGCGCCCTGAGCAACAGCAT |
| | CATGTACTTCAGCCACTTCGTGCCCGTGTTTCTGCCCGCCAAGCCTACAACAACCCCTGC |
| | TCCTAGACCTCCTACACCAGCTCCTACAATCGCCAGCCAGCCTCTGTCTCTGAGGCCAGA |
| | AGCTTGTAGACCTGCTGCAGGCGGAGCCGTGCATACAAGAGGACTGGATTTCGCCTGCGA |
| | CATCTACATCTGGGCCCCTCTGGCTGGAACATGTGGTGTCCTGCTGCTGAGCCTGGTCAT |
| | CACCCTGTACTGCAACCACCGGCGGAGCAAGAGAAGCAGACTGCTGCACAGCGACTACAT |
| | GAACATGACCCCTAGACGGCCCGGACCTACCAGAAAGCACTACCAGCCTTACGCTCCTCC |
| | TAGAGACTTCGCCGCCTACCGGTCCAGAGTGAAGTTCAGCAGATCCGCCGATGCTCCCGC |
| | CTATCAGCAGGGACAGAACCAGCTGTACAACGAGCTGAACCTGGGGAGAAGAGAAGAGTA |
| | CGACGTGCTGGACAAGCGGAGAGGCAGAGATCCTGAGATGGGCGGCAAGCCCAGACGGAA |
| | GAATCCTCAAGAGGGCCTGTATAATGAGCTGCAGAAAGACAAGATGGCCGAGGCCTACAG |
| | CGAGATCGGAATGAAGGGCGAGCGCAGAAGAGGCAAGGGACACGATGGACTGTACCAGGG |
| | CCTGAGCACCGCCACCAAGGATACCTATGATGCCCTGCACATGCAGGCCCTGCCTCCAAG |
| | Ataaagatctagatccggattagtccaatttgttaaagacaggatatcagtggtccaggc |
| | tctagttttgactcaacaatatcaccagctgaagcctatagagtacgagccatagataaa |
| | ataaaagattttatttagtctccagaaaaaggggggaatgaaagaccccacctgtaggtt |
| | tggcaagctagcttaagtaacgccattttgcaaggcatggaaaaatacataactgagaat |
| | agagaagttcagatcaaggtcaggaacagatggaacagctgaatatgggccaaacaggat |
| | atctgtggtaagcagttcctgccccggctcagggccaagaacagatggaacagctgaata |
| | tgggccaaacaggatatctgtggtaagcagttcctgccccggctcagggccaagaacaga |
| | tggtccccagatgcggtccagccctcagcagtttctagagaaccatcagatgtttccagg |
| | gtgccccaaggacctgaaatgaccctgtgccttatttgaactaaccaatcagttcgcttc |
| | tcgcttctgttcgcgcgcttctgctccccgagctcaataaaagagcccacaacccctcac |
| | tcggggcgccagtcctccgattgactgagtcgcccgggtacccgtgtatccaataaaccc |
| | tcttgcagttgcatccgacttgtggtctgctgttccttgggagggtctcctctgagtgat |
| | tgactacccgtcagcgggggtctttcacatgcagcatgtatcaaaattaatttggttttt |
| | tttcttaagtatttacattaaatggccatagtacttaaagttacattggcttccttgaaa |
| | taaacatggagtattcagaatgtgtcataaatatttctaattttaagatagtatctccat |
| | tggctttctactttttcttttatttttttttgtcctctgtcttccatttgttgttgttgt |
| | tgtttgtttgtttgtttgttggttggttggttaatttttttttaaagatcctacactata |
| | gttcaagctagactattagctactctgtaacccagggtgaccttgaagtcatgggtagcc |
| | tgctgttttagccttcccacatctaagattacaggtatgagctatcatttttggtatatt |
| | gattgattgattgattgatgtgtgtgtgtgtgattgtgtttgtgtgtgtgaTtgtgTaTa |
| | tgtgtgtatggTtgtgtgtgaTtgtgtgtatgtatgTTtgtgtgtgaTtgTgtgtgtgtg |
| | aTtgtgcatgtgtgtgtgtgtgaTtgtgtTtatgtgtatgaTtgtgtgtgtgtgtgtgtg |
| | tgtgtgtgtgtgtgtgtgtgtgtgtgtgttgtgTaTaTatatttatggtagtgagagGca |
| | acgctccggctcaggtgtcaggttggtttttgagacagagtctttcacttagcttggaat |
| | taattcactggccgtcgttttacaacgtcgtgactgggaaaaccctggcgttacccaact |
| | taatcgccttgcagcacatccccctttcgccagctggcgtaatagcgaagaggcccgcac |
| | cgatcgcccttcccaacagttgcgcagcctgaatggcgaatggcgcctgatgcggtattt |
| | tctccttacgcatctgtgcggtatttcacaccgcatatggtgcactctcagtacaatctg |
| | ctctgatgccgcatagttaagccagccccgacacccgccaacacccgctgacgcgccctg |
| | acgggcttgtctgctcccggcatccgcttacagacaagctgtgaccgtctccgggagctg |
| | catgtgtcagaggttttcaccgtcatcaccgaaacgcgcgagacgaaagggcctcgtgat |
| | acgcctatttttataggttaatgtcatgataataatggtttcttagacgtcaggtggcac |
| | ttttcggggaaatgtgcgcggaacccctatttgtttatttttctaaatacattcaaatat |
| | gtatccgctcatgagacaataaccctgataaatgcttcaataatattgaaaaaggaagag |
| | tatgagtattcaacatttccgtgtcgcccttattcccttttttgcggcattttgccttcc |
| | tgtttttgctcacccagaaacgctggtgaaagtaaaagatgctgaagatcagttgggtgc |
| | acgagtgggttacatcgaactggatctcaacagcggtaagatccttgagagttttcgccc |
| | cgaagaacgttttccaatgatgagcacttttaaagttctgctatgtggcgcggtattatc |
| | ccgtattgacgccgggcaagagcaactcggtcgccgcatacactattctcagaatgactt |
| | ggttgagtactcaccagtcacagaaaagcatcttacggatggcatgacagtaagagaatt |
| | atgcagtgctgccataaccatgagtgataacactgcggccaacttacttctgacaacgat |
| | cggaggaccgaaggagctaaccgcttttttgcacaacatgggggatcatgtaactcgcct |
| | tgatcgttgggaaccggagctgaatgaagccataccaaacgacgagcgtgacaccacgat |
| | gcctgtagcaatggcaacaacgttgcgcaaactattaactggcgaactacttactctagc |
| | ttcccggcaacaattaatagactggatggaggcggataaagttgcaggaccacttctgcg |
| | ctcggcccttccggctggctggtttattgctgataaatctggagccggtgagcgtgggtc |
| | tcgcggtatcattgcagcactggggccagatggtaagccccccgtatcgtagttatctac |
| | acgacggggagtcaggcaactatggatgaacgaaatagacagatcgctgagataggtgcc |
| | tcactgattaagcattggtaactgtcagaccaagtttactcatatatactttagattgat |
| | ttaaaacttcatttttaatttaaaaggatctaggtgaagatcctttttgataatctcatg |
| | accaaaatcccttaacgtgagttttcgttccactgagcgtcagaccccgtagaaaagatc |
| | aaaggatcttcttgagatcctttttttctgcgcgtaatctgctgcttgcaaacaaaaaaa |
| | ccaccgctaccagcggtggtttgtttgccggatcaagagctaccaactctttttccgaag |
| | gtaactggcttcagcagagcgcagataccaaatactgtccttctagtgtagccgtagtta |
| | ggccaccacttcaagaactctgtagcaccgcctacatacctcgctctgctaatcctgtta |
| | ccagtggctgctgccagtggcgataagtcgtgtcttaccgggttggactcaagacgatag |
| | ttaccggataaggcgcagcggtcgggctgaacggggggttcgtgcacacagcccagcttg |
| | gagcgaacgacctacaccgaactgagatacctacagcgtgagctatgagaaagcgccacg |
| | cttcccgaagggagaaaggcggacaggtatccggtaagcggcagggtcggaacaggagag |
| | cgcacgagggagcttccagggggaaacgcctggtatctttatagtcctgtcgggtttcgc |
| | cacctctgacttgagcgtcgatttttgtgatgctcgtcaggggggcggagcctatggaaa |
| | aacgccagcaacgcggcctttttacggttcctggccttttgctggccttttgctcacatg |
| | ttctttcctgcgttatcccctgattctgtggataaccgtattaccgcctttgagtgagct |
| | gataccgctcgccgcagccgaacgaccgagcgcagcgagtcagtgagcgaggaagcggaa |
| | gagcgcccaatacgcaaaccgcctctccccgcgcgttggccgattcattaatgcagctgg |
| | cacgacaggtttcccgactggaaagcgggcagtgagcgcaacgcaattaatgtgagttag |
| | ctcactcattaggcaccccaggctttacactttatgcttccggctcgtatgttgtgtgga |
| | attgtgagcggataacaatttcacacaggaaacagctatgaccatgattacgcc(SEQID |
| | NO:313) |
| SB06294 | aagctttgctcttaggagtttcctaatacatcccaaactcaaatatataaagcatttgac |
| (lgE (SS)- | ttgttctatgccctagggggcggggggaagctaagccagctttttttaacatttaaaatg |
| IL15 Tace10 | ttaattccattttaaatgcacagatgtttttatttcataagggtttcaatgtgcatgaat |
| (cleavage | gctgcaatattcctgttaccaaagctagtataaataaaaatagataaacgtggaaattac |
| site)-B7-1 | ttagagtttctgtcattaacgtttccttcctcagttgacaacataaatgcgctgctgaga |
| (TM)-E2A | agccagtttgcatctgtcaggatcaatttcccattatgccagtcatattaattactagtc |
| T2A-GM- | aattagttgatttttatttttgacatatacatgtgaaagaccccacctgtaggtttggca |
| CSF-Ra (SS) | agctagcttaagtaacgccattttgcaaggcatggaaaaatacataactgagaatagaaa |
| -aGPC3 | agttcagatcaaggtcaggaacagatggaacagctgaatatgggccaaacaggatatctg |
| hPY7 vL- | tggtaagcagttcctgccccggctcagggccaagaacagatggaacagctgaatatgggc |
| (GGGGS)3 | caaacaggatatctgtggtaagcagttcctgccccggctcagggccaagaacagatggtc |
| (SEQ ID NO: | cccagatgcggtccagccctcagcagtttctagagaaccatcagatgtttccagggtgcc |
| 223)-aGPC3 | ccaaggacctgaaatgaccctgtgccttatttgaactaaccaatcagttcgcttctcgct |
| hPY7 vH- | tctgttcgcgcgcttctgctccccgagctcaataaaagagcccacaacccctcactcggc |
| CD8 S2L | gcgccagtcctccgattgactgagtcgcccgggtacccgtgtatccaataaaccctcttg |
| (Hinge)- | cagttgcatccgacttgtggtctcgctgttccttgggagggtctcctctgagtgattgac |
| OX40 (TM)- | tacccgtcagcgggggtctttcatttgggggctcgtccgagatcgggagacccctgccca |
| OX40 (ICD)- | gggaccaccgacccaccaccgggaggtaagctggccagcaacttatctgtgtctgtccga |
| CD3z mut | ttgtctagtgtctatgactgattttatgcgcctgcgtcggtactagttagctaactagct |
| (ICD)) | ctgtatctggcggacccgtggtggaactgacgagttcggaacacccggccgcaaccctgg |
| | gagacgtcccagggacttcgggggccgtttttgtggcccgacctgagtcctaaaatcccg |
| | atcgtttaggactctttggtgcaccccccttagaggagggatatgtggttctggtaggag |
| | acgagaacctaaaacagttcccgcctccgtctgaatttttgctttcggtttgggaccgaa |
| | gccgcgccgcgcgtcttgtctgctgcagcatcgttctgtgttgtctctgtctgactgtgt |
| | ttctgtatttgtctgaaaatatggatcttatatggggcacccccgccccttgtaaacttc |
| | cctgaccctgacatgacaagagttactaacagcccctctctccaagctcacttacaggct |
| | ctctacttagtccagcacgaagtctggagacctctggcggcagcctaccaagaacaactg |
| | gaccgaccggtggtacctcacccttaccgagtcggcgacacagtgtgggtccgccgacac |
| | cagactaagaacctagaacctcgctggaaaggaccttacacagtcctgctgaccaccccc |
| | accgccctcaaagtagacggcatcgcagcttggatacacgccgcccacgtgaaggctgcc |
| | gaccccgggggtggaccatcctctagactgccggatccGCCGCCACCATGGACTGGACCT |
| | GGATCCTGTTTCTGGTGGCCGCTGCCACAAGAGTGCACAGCAATTGGGTCAACGTGATCA |
| | GCGACCTGAAGAAGATCGAGGACCTGATCCAGAGCATGCACATCGACGCCACACTGTACA |
| | CCGAGAGCGACGTGCACCCTAGCTGTAAAGTGACCGCCATGAAGTGCTTTCTGCTGGAAC |
| | TGCAAGTGATCAGCCTGGAAAGCGGCGACGCCAGCATCCACGACACCGTGGAAAACCTGA |
| | TCATCCTGGCCAACAACAGCCTGAGCAGCAACGGCAATGTGACCGAGTCCGGCTGCAAAG |
| | AGTGCGAGGAACTGGAAGAGAAGAATATCAAAGAGTTCCTGCAGAGCTTCGTGCACATCG |
| | TGCAGATGTTCATCAACACAAGCTCTGGCGGCGGAGGATCTGGCGGAGGTGGAAGCGGAG |
| | TTACACCCGAGCCTATCTTCAGCCTGATCGGAGGCGGTAGCGGAGGCGGAGGAAGTGGTG |
| | GCGGATCTCTGCAACTGCTGCCTAGCTGGGCCATCACACTGATCTCCGTGAACGGCATCT |
| | TCGTGATCTGCTGCCTGACCTACTGCTTCGCCCCTAGATGCAGAGAGCGGCGGAGAAACG |
| | AACGGCTGAGAAGAGAATCTGTGCGGCCCGTTCAGTGTACCAACTACGCCCTGCTGAAAC |
| | TGGCCGGCGACGTGGAATCTAATCCTGGACCTGGATCTGGCGAGGGACGCGGGAGTCTAC |
| | TGACGTGTGGAGACGTGGAGGAAAACCCTGGACCTATGCTGCTGCTGGTCACATCTCTGC |
| | TGCTGTGCGAGCTGCCCCATCCTGCCTTTCTGCTGATCCCTCACATGGACATCGTGATGA |
| | CACAGAGCCCCGATAGCCTGGCCGTGTCTCTGGGAGAAAGAGCCACCATCAACTGCAAGA |
| | GCAGCCAGAGCCTGCTGTACTCCAGCAACCAGAAGAACTACCTGGCCTGGTATCAGCAAA |
| | AGCCCGGCCAGCCTCCTAAGCTGCTGATCTATTGGGCCAGCTCCAGAGAAAGCGGCGTGC |
| | CCGATAGATTTTCTGGCTCTGGCAGCGGCACCGACTTCACCCTGACAATTTCTAGCCTGC |
| | AAGCCGAGGACGTGGCCGTGTACTACTGCCAGCAGTACTACAACTACCCTCTGACCTTCG |
| | GCCAGGGCACCAAGCTGGAAATCAAAGGCGGCGGAGGCTGGCGGAGGTGGAAGTGGCGGA |
| | GGCGGATCTGAAGTGCAGCTGGTTGAATCAGGTGGCGGCCTGGTTCAACCTGGCGGATCT |
| | CTGAGACTGAGCTGTGCCGCCAGCGGCTTCACCTTCAACAAGAACGCCATGAACTGGGTC |
| | CGACAGGCCCCTGGCAAAGGCCTTGAATGGGTCGGACGGATCCGGAACAAGACCAACAAC |
| | TACGCCACCTACTACGCCGACAGCGTGAAGGCCAGATTCACCATCAGCCGGGACGACAGC |
| | AAGAACAGCCTGTACCTGCAGATGAACTCCCTGAAAACCGAGGACACCGCCGTGTATTAT |
| | TGCGTGGCCGGCAACAGCTTTGCCTACTGGGGACAGGGAACCCTGGTCACCGTGTCTGCC |
| | ACAACAACCCCTGCTCCTAGACCTCCTACACCAGCTCCTACAATCGCCCTGCAGCCTCTG |
| | TCTCTGAGGCCAGAAGCTTGTAGACCAGCTGCTGGCGGAGCCGTGCATACAAGAGGACTG |
| | GACTTCGCCTGTGATGTGGCCGCCATTCTCGGACTGGGACTTGTTCTGGGACTGCTGGGA |
| | CCTCTGGCCATTCTGCTGGCTCTGTATCTGCTGCGGAGGGACCAAAGACTGCCTCCTGAT |
| | GCTCACAAGCCTCCAGGCGGAGGCAGCTTCAGAACCCCTATCCAAGAGGAACAGGCCGAC |
| | GCTCACAGCACCCTGGCCAAGATTAGAGTGAAGTTCAGCAGGAGCGCAGACGCCCCCGCG |
| | TACAAGCAGGGCCAGAACCAGCTCTATAACGAGCTCAATCTAGGACGAAGAGAGGAGTAC |
| | GATGTTTTGGACAAGAGACGTGGCCGGGACCCTGAGATGGGGGGAAAGCCGAGAAGGAAG |
| | AACCCTCAGGAAGGCCTGTACAATGAACTGCAGAAAGATAAGATGGCGGAGGCCTACAGT |
| | GAGATTGGGATGAAAGGCGAGCGCCGGAGGGGCAAGGGGCACGATGGCCTTTACCAGGGT |
| | CTCAGTACAGCCACCAAGGACACCTACGACGCCCTTCACATGCAGGCCCTGCCCCCTCGC |
| | taaagatctagatccggattagtccaatttgttaaagacaggatatcagtggtccaggct |
| | ctagttttgactcaacaatatcaccagctgaagcctatagagtacgagccatagataaaa |
| | taaaagattttatttagtctccagaaaaaggggggaatgaaagaccccacctgtaggttt |
| | ggcaagctagcttaagtaacgccattttgcaaggcatggaaaaatacataactgagaata |
| | gagaagttcagatcaaggtcaggaacagatggaacagctgaatatgggccaaacaggata |
| | tctgtggtaagcagttcctgccccggctcagggccaagaacagatggaacagctgaatat |
| | gggccaaacaggatatctgtggtaagcagttcctgccccggctcagggccaagaacagat |
| | ggtccccagatgcggtccagccctcagcagtttctagagaaccatcagatgtttccaggg |
| | tgccccaaggacctgaaatgaccctgtgccttatttgaactaaccaatcagttcgcttct |
| | cgcttctgttcgcgcgcttctgctccccgagctcaataaaagagcccacaacccctcact |
| | cggggcgccagtcctccgattgactgagtcgcccgggtacccgtgtatccaataaaccct |
| | cttgcagttgcatccgacttgtggtctcgctgttccttgggagggtctcctctgagtgat |
| | tgactacccgtcagcgggggtctttcacatgcagcatgtatcaaaattaatttggttttt |
| | tttcttaagtatttacattaaatggccatagtacttaaagttacattggcttccttgaaa |
| | taaacatggagtattcagaatgtgtcataaatatttctaattttaagatagtatctccat |
| | tggctttctactttttcttttatttttttttgtcctctgtcttccatttgttgttgttgt |
| | tgtttgtttgtttgtttgttggttggttggttaatttttttttaaagatcctacactata |
| | gttcaagctagactattagctactctgtaacccagggtgaccttgaagtcatgggtagcc |
| | tgctgttttagccttcccacatctaagattacaggtatgagctatcatttttggtatatt |
| | gattgattgattgattgatgtgtgtgtgtgtgattgtgtttgtgtgtgtgaTtgtgTaTa |
| | tgtgtgtatggTtgtgtgtgaTtgtgtgtatgtatgTTtgtgtgtgaTtgTgtgtgtgtg |
| | aTtgtgcatgtgtgtgtgtgtgaTtgtgtTtatgtgtatgaTtgtgtgtgtgtgtgtgtg |
| | tgtgtgtgtgtgtgtgtgtgtgtgtgtgttgtgTaTaTatatttatggtagtgagagGca |
| | acgctccggctcaggtgtcaggttggtttttgagacagagtctttcacttagcttggaat |
| | taattcactggccgtcgttttacaacgtcgtgactgggaaaaccctggcgttacccaact |
| | taatcgccttgcagcacatccccctttcgccagctggcgtaatagcgaagaggcccgcac |
| | cgatcgcccttcccaacagttgcgcagcctgaatggcgaatggcgcctgatgcggtattt |
| | tctccttacgcatctgtgcggtatttcacaccgcatatggtgcactctcagtacaatctg |
| | ctctgatgccgcatagttaagccagccccgacacccgccaacacccgctgacgcgccctg |
| | acgggcttgtctgctcccggcatccgcttacagacaagctgtgaccgtctccgggagctg |
| | catgtgtcagaggttttcaccgtcatcaccgaaacgcgcgagacgaaagggcctcgtgat |
| | acgcctatttttataggttaatgtcatgataataatggtttcttagacgtcaggtggcac |
| | ttttcggggaaatgtgcgcggaacccctatttgtttatttttctaaatacattcaaatat |
| | gtatccgctcatgagacaataaccctgataaatgcttcaataatattgaaaaaggaagag |
| | tatgagtattcaacatttccgtgtcgcccttattcccttttttgcggcattttgccttcc |
| | tgtttttgctcacccagaaacgctggtgaATaagtaaaagatgctgaagatcagttgggt |
| | gcacgagtgggttacatgaactggatctcaacagcggtaagatccttgagagttttcgcc |
| | ccgaagaacgttttccaatgatgagcacttttaaagttctgctatgtggcgcggtattat |
| | cccgtattgacgccgggcaagagcaactcggtcgccgcatacactattctcagaatgact |
| | tggttgagtactcaccagtcacagaaaagcatcttacggatggcatgacagtaagagaat |
| | tatgcagtgctgccataaccatgagtgataacactgcggccaacttacttctgacaacga |
| | tcggaggaccgaaggagctaaccgcttttttgcacaacatgggggatcatgtaactcgcc |
| | ttgatcgttgggaaccggagctgaatgaagccataccaaacgacgagcgtgacaccacga |
| | tgcctgtagcaatggcaacaacgttgcgcaaactattaactggcgaactacttactctag |
| | cttcccggcaacaattaatagactggatggaggcggataaagttgcaggaccacttctgc |
| | gctcggcccttccggctggctggtttattgctgataaatctggagccggtgagcgtgggt |
| | ctcgcggtatcattgcagcactggggccagatggtaagccctcccgtatcgtagttatct |
| | acacgacggggagtcaggcaactatggatgaacgaaatagacagatcgctgagataggtg |
| | cctcactgattaagcattggtaactgtcagaccaagtttactcatatatactttagattg |
| | atttaaaacttcatttttaatttaaaaggatctaggtgaagatcctttttgataatctca |
| | tgaccaaaatcccttaacgtgagttttcgttccactgagcgtcagaccccgtagaaaaga |
| | tcaaaggatcttcttgagatcctttttttctgcgcgtaatctgctgcttgcaaacaaaaa |
| | aaccaccgctaccagcggtggtttgtttgccggatcaagagctaccaactctttttccga |
| | aggtaactggcttcagcagagcgcagataccaaatactgtccttctagtgtagccgtagt |
| | taggccaccacttcaagaactctgtagcaccgcctacatacctcgctctgctaatcctgt |
| | taccagtggctgctgccagtggcgataagtcgtgtcttaccgggttggactcaagacgat |
| | agttaccggataaggcgcagcggtcgggctgaacggggggttcgtgcacacagcccagct |
| | tggagcgaacgacctacaccgaactgagatacctacagcgtgagctatgagaaagcgcca |
| | cgcttcccgaagggagaaaggcggacaggtatccggtaagcggcagggtcggaacaggag |
| | agcgcacgagggagcttccagggggaaacgcctggtatctttatagtcctgtcgggtttc |
| | gccacctctgacttgagcgtcgatttttgtgatgctcgtcaggggggcggagcctatgga |
| | aaaacgccagcaacgcggcctttttacggttcctggccttttgctggccttttgctcaca |
| | tgttctttcctgcgttatcccctgattctgtggataaccgtattaccgcctttgagtgag |
| | ctgataccgctcgccgcagccgaacgaccgagcgcagcgagtcagtgagcgaggaagcgg |
| | aagagcgcccaatacgcaaaccgcctctccccgcgcgttggccgattcattaatgcagct |
| | ggcacgacaggtttcccgactggaaagcgggcagtgagcgcaacgcaattaatgtgagtt |
| | agctcactcattaggcaccccaggctttacactttatgcttccggctcgtatgttgtgtg |
| | gaattgtgagcggataacaatttcacacaggaaacagctatgaccatgattacgcc(SEQ |
| | IDNO:314) |
| SB06692 | aagcttggaattcgagcttgcatgcctgcaggtcgttacataacttacggtaaatggccc |
| | gcctggctgaccgcccaacgacccccgcccattgacgtcaataatgacgtatgttcccat |
| | agtaacgccaatagggactttccattgacgtcaatgggtggagtatttacggtaaactgc |
| | ccacttggcagtacatcaagtgtatcatatgccaagtacgccccctattgacgtcaatga |
| | cggtaaatggcccgcctggcattatgcccagtacatgaccttatgggactttcctacttg |
| | gcagtacatctacgtattagtcatcgctattaccatggtgatgcggttttggcagtacat |
| | caatgggcgtggatagcggtttgactcacggggatttccaagtctccaccccattgacgt |
| | caatgggagtttgttttggcaccaaaatcaacgggactttccaaaatgtcgtaacaactc |
| | cgccccattgacgcaaatgggcggtaggcgtgtacggtgggaggtctatataagcagagc |
| | tcaataaaagagcccacaacccctcactcggcgcgccagtcctccgattgactgagtcgc |
| | ccgggtacccgtgtatccaataaaccctcttgcagttgcatccgacttgtggtctcgctg |
| | ttccttgggagggtctcctctgagtgattgactacccgtcagcgggggtctttcatttgg |
| | gggctcgtccgagatcgggagacccctgcccagggaccaccgacccaccaccgggaggta |
| | agctggccagcaacttatctgtgtctgtccgattgtctagtgtctatgactgattttatg |
| | cgcctgcgtcggtactagttagctaactagctctgtatctggcggacccgtggtggaact |
| | gacgagttcggaacacccggccgcaaccctgggagacgtcccagggacttcgggggccgt |
| | ttttgtggcccgacctgagtcctaaaatcccgatcgtttaggactctttggtgcaccccc |
| | cttagaggagggatatgtggttctggtaggagacgagaacctaaaacagttcccgcctcc |
| | gtctgaatttttgctttcggtttgggaccgaagccgcgccgcgcgtcttgtctgctgcag |
| | catcgttctgtgttgtctctgtctgactgtgtttctgtatttgtctgaaaatatgggccc |
| | cccctcgaggtaacgccattttgcaaggcatggaaaaataccaaaccaagaatagagaag |
| | ttcagatcaagggcgggtacatgaaaatagctaacgttgggccaaacaggatatctgcgg |
| | tgagcagtttcggccccggcccggggccaagaacagatggtcaccgcagtttcggccccg |
| | gcccgaggccaagaacagatggtccccagatatggcccaaccctcagcagtttcttaaga |
| | cccatcagatgtttccaggctcccccaaggacctgaaatgaccctgcgccttatttgaat |
| | taaccaatcagcctgcttctcgcttctgttcgcgcgcttctgcttcccgagctctataaa |
| | agagctcacaacccctcactcggcgcgccagtcctccgacagactgagtcgcccgggGCC |
| | GCCACCATGGACTGGACCTGGATCCTGTTTCTGGTGGCCGCTGCCACAAGAGTGCACAGC |
| | AATTGGGTCAACGTGATCAGCGACCTGAAGAAGATCGAGGACCTGATCCAGAGCATGCAC |
| | ATCGACGCCACACTGTACACCGAGAGCGACGTGCACCCTAGCTGTAAAGTGACCGCCATG |
| | AAGTGCTTTCTGCTGGAACTGCAAGTGATCAGCCTGGAAAGCGGCGACGCCAGCATCCAC |
| | GACACCGTGGAAAACCTGATCATCCTGGCCAACAACAGCCTGAGCAGCAACGGCAATGTG |
| | ACCGAGTCCGGCTGCAAAGAGTGCGAGGAACTGGAAGAGAAGAATATCAAAGAGTTCCTG |
| | CAGAGCTTCGTGCACATCGTGCAGATGTTCATCAACACAAGCCCCAGAGCCGAGGCTCTG |
| | AAAGGCGGATCAGGCGGCGGTGGTAGTGGAGGCGGAGGCTCAGGCGGCGGAGGTTCCGGA |
| | GGTGGCGGTTCCGGCGGAGGATCTCTTCAATTGCTGCCTAGCTGGGCCATCACACTGATC |
| | TCCGTGAACGGCATCTTCGTGATCTGCTGCCTGACCTACTGCTTCGCCCCTAGATGCAGA |
| | GAGCGGAGAAGAAACGAGCGGCTGAGAAGAGAAAGCGTGCGGCCTGTGGGTAGCGGCCAG |
| | TGTACCAACTACGCCCTGCTGAAACTGGCCGGCGACGTGGAATCTAATCCTGGACCTGGA |
| | TCTGGCGAGGGACGCGGGAGTCTACTGACGTGTGGAGACGTGGAGGAAAACCCTGGACCT |
| | ATGCTGCTGCTGGTCACATCTCTGCTGCTGTGCGAGCTGCCCCATCCTGCCTTTCTGCTG |
| | ATCCCTCACATGGACATCGTGATGACACAGAGCCCCGATAGCCTGGCCGTGTCTCTGGGA |
| | GAAAGAGCCACCATCAACTGCAAGAGCAGCCAGAGCCTGCTGTACTCCAGCAACCAGAAG |
| | AACTACCTGGCCTGGTATCAGCAAAAGCCCGGCCAGCCTCCTAAGCTGCTGATCTATTGG |
| | GCCAGCTCCAGAGAAAGCGGCGTGCCCGATAGATTTTCTGGCTCTGGCAGCGGCACCGAC |
| | TTCACCCTGACAATTTCTAGCCTGCAAGCCGAGGACGTGGCCGTGTACTACTGCCAGCAG |
| | TACTACAACTACCCTCTGACCTTCGGCCAGGGCACCAAGCTGGAAATCAAAGGCGGCGGA |
| | GGATCTGGCGGAGGTGGAAGTGGCGGAGGCGGATCTGAAGTGCAGCTGGTTGAATCAGGT |
| | GGCGGCCTGGTTCAACCTGGCGGATCTCTGAGACTGAGCTGTGCCGCCAGCGGCTTCACC |
| | TTCAACAAGAACGCCATGAACTGGGTCCGACAGGCCCCTGGCAAAGGCCTTGAATGGGTC |
| | GGACGGATCCGGAACAAGACCAACAACTACGCCACCTACTACGCCGACAGCGTGAAGGCC |
| | AGATTCACCATCAGCCGGGACGACAGCAAGAACAGCCTGTACCTGCAGATGAACTCCCTG |
| | AAAACCGAGGACACCGCCGTGTATTATTGCGTGGCCGGCAACAGCTTTGCCTACTGGGGA |
| | CAGGGAACCCTGGTCACCGTGTCTGCCACAACAACCCCTGCTCCTAGACCTCCTACACCA |
| | GCTCCTACAATCGCCCTGCAGCCTCTGTCTCTGAGGCCAGAAGCTTGTAGACCAGCTGCT |
| | GGCGGAGCCGTGCATACAAGAGGACTGGACTTCGCCTGTGATGTGGCCGCCATTCTCGGA |
| | CTGGGACTTGTTCTGGGACTGCTGGGACCTCTGGCCATTCTGCTGGCTCTGTATCTGCTG |
| | CGGAGGGACCAAAGACTGCCTCCTGATGCTCACAAGCCTCCAGGCGGAGGCAGCTTCAGA |
| | ACCCCTATCCAAGAGGAACAGGCCGACGCTCACAGCACCCTGGCCAAGATTAGAGTGAAG |
| | TTCAGCAGAAGCGCCGACGCACCCGCCTATAAGCAGGGACAGAACCAGCTGTACAACGAG |
| | CTGAACCTGGGGAGAAGAGAAGAGTACGACGTGCTGGACAAGCGGAGAGGCAGAGATCCT |
| | GAGATGGGCGGCAAGCCCAGACGGAAGAATCCTCAAGAGGGCCTGTATAATGAGCTGCAG |
| | AAAGACAAGATGGCCGAGGCCTACAGCGAGATCGGAATGAAGGGCGAGCGCAGAAGAGGC |
| | AAGGGACACGATGGACTGTACCAGGGCCTGAGCACCGCCACCAAGGATACCTATGATGCC |
| | CTGCACATGCAGGCCCTGCCTCCAAGAtaaggatccggattagtccaatttgttaaagac |
| | aggatgggctgcaggaattccgataatcaacctctggattacaaaatttgtgaaagattg |
| | actggtattcttaactatgttgctccttttacgctatgtggatacgctgctttaatgcct |
| | ttgtatcatgctattgcttcccgtatggctttcattttctcctccttgtataaatcctgg |
| | ttgctgtctctttatgaggagttgtggcccgttgtcaggcaacgtggcgtggtgtgcact |
| | gtgtttgctgacgcaacccccactggttggggcattgccaccacctgtcagctcctttcc |
| | gggactttcgctttccccctccctattgccacggcggaactcatcgccgcctgccttgcc |
| | cgctgctggacaggggctcggctgttgggcactgacaattccgtggtgttgtcggggaag |
| | ctgacgtcctttccatggctgctcgcctgtgttgccacctggattctgcgcgggacgtcc |
| | ttctgctacgtcccttcggccctcaatccagcggaccttccttcccgcggcctgctgccg |
| | gctctgcggcctcttccgcgtcttcgccttcgccctcagacgagtcggatctccctttgg |
| | gccgcctccccgcctggagaattcgatatcagtggtccaggctctagttttgactcaaca |
| | atatcaccagctgaagcctatagagtacgagccatagataaaataaaagattttatttag |
| | tctccagaaaaaggggggaatgaaagaccccacctgtaggtttggcaagctagcaataaa |
| | agagcccacaacccctcactcggggcgccagtcctccgattgactgagtcgcccggccgc |
| | ttcgagcagacatgataagatacattgatgagtttggacaaaccacaactagaatgcagt |
| | gaaaaaaatgctttatttgtgaaatttgtgatgctattgctttatttgtaaccattataa |
| | gctgcaataaacaagttaacaacaacaattgcattcattttatgtttcaggttcaggggg |
| | agatgtgggaggttttttaaagcaagtaaaacctctacaaatgtggtaaaatcgataagg |
| | atcgggtacccgtgtatccaataaaccctcttgcagttgcatccgacttgtggtctcgct |
| | gttccttgggagggtctcctctgagtgattgactacccgtcagcgggggtctttcacaca |
| | tgcagcatgtatcaaaattaatttggttttttttcttaagctgtgccttctagttgccag |
| | ccatctgttgtttgcccctcccccgtgccttccttgaccctggaaggtgccactcccact |
| | gtcctttcctaataaaatgaggaaattgcatcgcattgtctgagtaggtgtcattctatt |
| | ctggggggtggggggggcaggacagcaagggggaggattgggaagacaatagcaggcatg |
| | ctggggatgcggtgggctctatggagatcccgcggtacctcgcgaatgcatctagatcca |
| | atggcctttttggcccagacatgataagatacattgatgagtttggacaaaccacaacta |
| | gaatgcagtgaaaaaaatgctttatttgtgaaatttgtgatgctattgctttatttgtaa |
| | ccattataagctgcaataaacaagttgcggccgcttagccctcccacacataaccagagg |
| | gcagcaattcacgaatcccaactgccgtcggctgtccatcactgtccttcactatggctt |
| | tgatcccaggatgcagatcgagaagcacctgtcggcaccgtccgcaggggctcaagatgc |
| | ccctgttctcatttccgatcgcgacgatacaagtcaggttgccagctgccgcagcagcag |
| | cagtgcccagcaccacgagttctgcacaaggtcccccagtaaaatgatatacattgacac |
| | cagtgaagatgcggccgtcgctagagagagctgcgctggcgacgctgtagtcttcagaga |
| | tggggatgctgttgattgtagccgttgctctttcaatgagggtggattcttcttgagaca |
| | aaggcttggccatgcggccgccgctcggtgttcgaggccacacgcgtcaccttaatatgc |
| | gaagtggacctcggaccgcgccgccccgactgcatctgcgtgttcgaattcgccaatgac |
| | aagacgctgggggggtttgtgtcatcatagaactaaagacatgcaaatatatttcttccg |
| | gggggtaccggcctttttggccATTGGatcggatctggccaaaaaggcccttaagtattt |
| | acattaaatggccatagtacttaaagttacattggcttccttgaaataaacatggagtat |
| | tcagaatgtgtcataaatatttctaattttaagatagtatctccattggctttctacttt |
| | ttcttttatttttttttgtcctctgtcttccatttgttgttgttgttgtttgtttgtttg |
| | tttgttggttggttggttaatttttttttaaagatcctacactatagttcaagctagact |
| | attagctactctgtaacccagggtgaccttgaagtcatgggtagcctgctgttttagcct |
| | tcccacatctaagattacaggtatgagctatcatttttggtatattgattgattgattga |
| | ttgatgtgtgtgtgtgtgattgtgtttgtgtgtgtgaTtgtgTaTatgtgtgtatggTtg |
| | tgtgtgaTtgtgtgtatgtatgTTtgtgtgtgaTtgTgtgtgtgtgaTtgtgcatgtgtg |
| | tgtgtgtgaTtgtgtTtatgtgtatgaTtgtgtgtgtgtgtgtgtgtgtgtgtgtgtgtg |
| | tgtgtgtgtgtgtgttgtgTaTaTatatttatggtagtgagagGcaacgctccggctcag |
| | gtgtcaggttggtttttgagacagagtctttcacttagcttggaattcactggccgtcgt |
| | tttacaacgtcgtgactgggaaaaccctggcgttacccaacttaatcgccttgcagcaca |
| | tccccctttcgccagctggcgtaatagcgaagaggcccgcaccgatcgcccttcccaaca |
| | gttgcgcagcctgaatggcgaatggcgcctgatgcggtattttctccttacgcatctgtg |
| | cggtatttcacaccgcatatggtgcactctcagtacaatctgctctgatgccgcatagtt |
| | aagccagccccgacacccgccaacacccgctgacgcgccctgacgggcttgtctgctccc |
| | ggcatccgcttacagacaagctgtgaccgtctccgggagctgcatgtgtcagaggttttc |
| | accgtcatcaccgaaacgcgcgagacgaaagggcctcgtgatacgcctatttttataggt |
| | taatgtcatgataataatggtttcttagacgtcaggtggcacttttcggggaaatgtgcg |
| | cggaacccctatttgtttatttttctaaatacattcaaatatgtatccgctcatgagaca |
| | ataaccctgataaatgcttcaataatattgaaaaaggaagagtatgagtattcaacattt |
| | ccgtgtcgcccttattcccttttttgcggcattttgccttcctgtttttgctcacccaga |
| | aacgctggtgaaagtaaaagatgctgaagatcagttgggtgcacgagtgggttacatcga |
| | actggatctcaacagcggtaagatccttgagagttttcgccccgaagaacgttttccaat |
| | gatgagcacttttaaagttctgctatgtggcgcggtattatcccgtattgacgccgggca |
| | agagcaactcggtcgccgcatacactattctcagaatgacttggttgagtactcaccagt |
| | cacagaaaagcatcttacggatggcatgacagtaagagaattatgcagtgctgccataac |
| | catgagtgataacactgcggccaacttacttctgacaacgatcggaggaccgaaggagct |
| | aaccgcttttttgcacaacatgggggatcatgtaactcgccttgatcgttgggaaccgga |
| | gctgaatgaagccataccaaacgacgagcgtgacaccacgatgcctgtagcaatggcaac |
| | aacgttgcgcaaactattaactggcgaactacttactctagcttcccggcaacaattaat |
| | agactggatggaggcggataaagttgcaggaccacttctgcgctcggcccttccggctgg |
| | ctggtttattgctgataaatctggagccggtgagcgtgggtctcgcggtatcattgcagc |
| | actggggccagatggtaagccctcccgtatcgtagttatctacacgacggggagtcaggc |
| | aactatggatgaacgaaatagacagatcgctgagataggtgcctcactgattaagcattg |
| | gtaactgtcagaccaagtttactcatatatactttagattgatttaaaacttcattttta |
| | atttaaaaggatctaggtgaagatcctttttgataatctcatgaccaaaatcccttaacg |
| | tgagttttcgttccactgagcgtcagaccccgtagaaaagatcaaaggatcttcttgaga |
| | tcctttttttctgcgcgtaatctgctgcttgcaaacaaaaaaaccaccgctaccagcggt |
| | ggtttgtttgccggatcaagagctaccaactctttttccgaaggtaactggcttcagcag |
| | agcgcagataccaaatactgtccttctagtgtagccgtagttaggccaccacttcaagaa |
| | ctctgtagcaccgcctacatacctcgctctgctaatcctgttaccagtggctgctgccag |
| | tggcgataagtcgtgtcttaccgggttggactcaagacgatagttaccggataaggcgca |
| | gcggtcgggctgaacggggggttcgtgcacacagcccagcttggagcgaacgacctacac |
| | cgaactgagatacctacagcgtgagctatgagaaagcgccacgcttcccgaagggagaaa |
| | ggcggacaggtatccggtaagcggcagggtcggaacaggagagcgcacgagggagcttcc |
| | agggggaaacgcctggtatctttatagtcctgtcgggtttcgccacctctgacttgagcg |
| | tcgatttttgtgatgctcgtcaggggggcggagcctatggaaaaacgccagcaacgcggc |
| | ctttttacggttcctggccttttgctggccttttgctcacatgttctttcctgcgttatc |
| | ccctgattctgtggataaccgtattaccgcctttgagtgagctgataccgctcgccgcag |
| | ccgaacgaccgagcgcagcgagtcagtgagcgaggaagcggaagagcgcccaatacgcaa |
| | accgcctctccccgcgcgttggccgattcattaatgcagctggcacgacaggtttcccga |
| | ctggaaagcgggcagtgagcgcaacgcaattaatgtgagttagctcactcattaggcacc |
| | ccaggctttacactttatgcttccggctcgtatgttgtgtggaattgtgagcggataaca |
| | atttcacacaggaaacagctatgaccatgattacgcc (SEQ ID NO: 315) |
| SB06261 | aagcttgaattcgagcttgcatgcctgcaggtcgttacataacttacggtaaatggcccg |
| | cctggctgaccgcccaacgacccccgcccattgacgtcaataatgacgtatgttcccata |
| | gtaacgccaatagggactttccattgacgtcaatgggtggagtatttacggtaaactgcc |
| | cacttggcagtacatcaagtgtatcatatgccaagtacgccccctattgacgtcaatgac |
| | ggtaaatggcccgcctggcattatgcccagtacatgaccttatgggactttcctacttgg |
| | cagtacatctacgtattagtcatcgctattaccatggtgatgcggttttggcagtacatc |
| | aatgggcgtggatagcggtttgactcacggggatttccaagtctccaccccattgacgtc |
| | aatgggagtttgttttggcaccaaaatcaacgggactttccaaaatgtcgtaacaactcc |
| | gccccattgacgcaaatgggggtaggcgtgtacggtgggaggtctatataagcagagctc |
| | aataaaagagcccacaacccctcactcggcgcgccagtcctccgattgactgagtcgccc |
| | gggtacccgtgtatccaataaaccctcttgcagttgcatccgacttgtggtctcgctgtt |
| | ccttgggagggtctcctctgagtgattgactacccgtcagcgggggtctttcatttgggg |
| | gctcgtccgagatcgggagacccctgcccagggaccaccgacccaccaccgggaggtaag |
| | ctggccagcaacttatctgtgtctgtccgattgtctagtgtctatgactgattttatgcg |
| | cctgcgtcggtactagttagctaactagctctgtatctggcggacccgtggtggaactga |
| | cgagttcggaacacccggccgcaaccctgggagacgtcccagggacttcgggggccgttt |
| | ttgtggcccgacctgagtcctaaaatcccgatcgtttaggactctttggtgcacccccct |
| | tagaggagggatatgtggttctggtaggagacgagaacctaaaacagttcccgcctccgt |
| | ctgaatttttgctttcggtttgggaccgaagccgcgcgcgcgtcttgtctgctgcagcat |
| | cgttctgtgttgtctctgtctgactgtgtttctgtatttgtctgaaaatatgggcccccc |
| | ctcgaggtaacgccattttgcaaggcatggaaaaataccaaaccaagaatagagaagttc |
| | agatcaagggcgggtacatgaaaatagctaacgttgggccaaacaggatatctgcggtga |
| | gcagtttcggccccggcccggggccaagaacagatggtcaccgcagtttcggccccggcc |
| | cgaggccaagaacagatggtccccagatatggcccaaccctcagcagtttcttaagaccc |
| | atcagatgtttccaggctcccccaaggacctgaaatgaccctgcgccttatttgaattaa |
| | ccaatcagcctgcttctcgcttctgttcgcgcgcttctgcttcccgagctctataaaaga |
| | gctcacaacccctcactcggcgcgccagtcctccgacagactgagtcgcccgggGCCGCC |
| | ACCATGGACTGGACCTGGATCCTGTTTCTGGTGGCCGCTGCCACAAGAGTGCACAGCAAT |
| | TGGGTCAACGTGATCAGCGACCTGAAGAAGATCGAGGACCTGATCCAGAGCATGCACATC |
| | GACGCCACACTGTACACCGAGAGCGACGTGCACCCTAGCTGTAAAGTGACCGCCATGAAG |
| | TGCTTTCTGCTGGAACTGCAAGTGATCAGCCTGGAAAGCGGCGACGCCAGCATCCACGAC |
| | ACCGTGGAAAACCTGATCATCCTGGCCAACAACAGCCTGAGCAGCAACGGCAATGTGACC |
| | GAGTCCGGCTGCAAAGAGTGCGAGGAACTGGAAGAGAAGAATATCAAAGAGTTCCTGCAG |
| | AGCTTCGTGCACATCGTGCAGATGTTCATCAACACAAGCTCTGGCGGCGGAGGATCTGGC |
| | GGAGGTGGAAGCGGAGTTACACCCGAGCCTATCTTCAGCCTGATCGGAGGCGGTAGCGGA |
| | GGCGGAGGAAGTGGTGGCGGATCTCTGCAACTGCTGCCTAGCTGGGCCATCACACTGATC |
| | TCCGTGAACGGCATCTTCGTGATCTGCTGCCTGACCTACTGCTTCGCCCCTAGATGCAGA |
| | GAGCGGCGGAGAAACGAACGGCTGAGAAGAGAATCTGTGCGGCCCGTTGGTAGCGGCCAG |
| | TGTACCAACTACGCCCTGCTGAAACTGGCCGGCGACGTGGAATCTAATCCTGGACCTGGA |
| | TCTGGCGAGGGACGCGGGAGTCTACTGACGTGTGGAGACGTGGAGGAAAACCCTGGACCT |
| | ATGCTGCTGCTGGTCACATCTCTGCTGCTGTGCGAGCTGCCCCATCCTGCCTTTCTGCTG |
| | ATCCCTCACATGGAAGTGCAGCTGGTGGAATCTGGCGGAGGACTGGTTCAACCTGGCGGC |
| | TCTCTGAGACTGTCTTGTGCCGCCAGCGGCTTCACCTTCAACAAGAACGCCATGAACTGG |
| | GTCCGACAGGCCCCTGGCAAAGGCCTTGAATGGGTCGGACGGATCCGGAACAAGACCAAC |
| | AACTACGCCACCTACTACGCCGACAGCGTGAAGGCCAGGTTCACCATCTCCAGAGATGAC |
| | AGCAAGAACAGCCTGTACCTGCAGATGAACTCCCTGAAAACCGAGGACACCGCCGTGTAC |
| | TATTGCGTGGCCGGCAATAGCTTTGCCTACTGGGGACAGGGCACCCTGGTTACAGTTTCT |
| | GCTGGCGGCGGAGGAAGCGGAGGCGGAGGATCCGGTGGTGGTGGATCTGACATCGTGATG |
| | ACACAGAGCCCCGATAGCCTGGCCGTGTCTCTGGGAGAAAGAGCCACCATCAACTGCAAG |
| | AGCAGCCAGAGCCTGCTGTACTCCAGCAACCAGAAGAACTACCTGGCCTGGTATCAGCAA |
| | AAGCCCGGCCAGCCTCCTAAGCTGCTGATCTATTGGGCCAGCTCCAGAGAAAGCGGCGTG |
| | CCCGATAGATTTTCTGGCTCTGGCAGCGGCACCGACTTCACCCTGACAATTTCTAGCCTG |
| | CAAGCCGAGGACGTGGCCGTGTATTACTGCCAGCAGTACTACAACTACCCTCTGACCTTC |
| | GGCCAGGGCACCAAGCTGGAAATCAAATCTGGCGCCCTGAGCAACAGCATCATGTACTTC |
| | AGCCACTTCGTGCCCGTGTTTCTGCCCGCCAAGCCTACAACAACCCCTGCTCCTAGACCT |
| | CCTACACCAGCTCCTACAATCGCCAGCCAGCCTCTGTCTCTGAGGCCAGAAGCTTGTAGA |
| | CCTGCTGCAGGCGGAGCCGTGCATACAAGAGGACTGGATTTCGCCTGCGACATCTACATC |
| | TGGGCCCCTCTGGCTGGAACATGTGGTGTCCTGCTGCTGAGCCTGGTCATCACCCTGTAC |
| | TGCAACCACCGGCGGAGCAAGAGAAGCAGACTGCTGCACAGCGACTACATGAACATGACC |
| | CCTAGACGGCCCGGACCTACCAGAAAGCACTACCAGCCTTACGCTCCTCCTAGAGACTTC |
| | GCCGCCTACCGGTCCAGAGTGAAGTTCAGCAGATCCGCCGATGCTCCCGCCTATCAGCAG |
| | GGACAGAACCAGCTGTACAACGAGCTGAACCTGGGGAGAAGAGAAGAGTACGACGTGCTG |
| | GACAAGCGGAGAGGCAGAGATCCTGAGATGGGCGGCAAGCCCAGACGGAAGAATCCTCAA |
| | GAGGGCCTGTATAATGAGCTGCAGAAAGACAAGATGGCCGAGGCCTACAGCGAGATCGGA |
| | ATGAAGGGCGAGCGCAGAAGAGGCAAGGGACACGATGGACTGTACCAGGGCCTGAGCACC |
| | GCCACCAAGGATACCTATGATGCCCTGCACATGCAGGCCCTGCCTCCAAGAtaaggatcc |
| | ggattagtccaatttgttaaagacaggatgggctgcaggaattccgataatcaacctctg |
| | gattacaaaatttgtgaaagattgactggtattcttaactatgttgctccttttacgcta |
| | tgtggatacgctgctttaatgcctttgtatcatgctattgcttcccgtatggctttcatt |
| | ttctcctccttgtataaatcctggttgctgtctctttatgaggagttgtggcccgttgtc |
| | aggcaacgtggcgtggtgtgcactgtgtttgctgacgcaacccccactggttggggcatt |
| | gccaccacctgtcagctcctttccgggactttcgctttccccctccctattgccacggcg |
| | gaactcatcgccgcctgccttgcccgctgctggacaggggctcggctgttgggcactgac |
| | aattccgtggtgttgtcggggaagctgacgtcctttccatggctgctcgcctgtgttgcc |
| | acctggattctgcgcgggacgtccttctgctacgtcccttcggccctcaatccagcggac |
| | cttccttcccgcggcctgctgccggctctgcggcctcttccgcgtcttcgccttcgccct |
| | cagacgagtcggatctccctttgggccgcctccccgcctggagaattcgatatcagtggt |
| | ccaggctctagttttgactcaacaatatcaccagctgaagcctatagagtacgagccata |
| | gataaaataaaagattttatttagtctccagaaaaaggggggaatgaaagaccccacctg |
| | taggtttggcaagctagcaataaaagagcccacaacccctcactcggggcgccagtcctc |
| | cgattgactgagtcgcccggccgcttcgagcagacatgataagatacattgatgagtttg |
| | gacaaaccacaactagaatgcagtgaaaaaaatgctttatttgtgaaatttgtgatgcta |
| | ttgctttatttgtaaccattataagctgcaataaacaagttaacaacaacaattgcattc |
| | attttatgtttcaggttcagggggagatgtgggaggttttttaaagcaagtaaaacctct |
| | acaaatgtggtaaaatcgataaggatcgggtacccgtgtatccaataaaccctcttgcag |
| | ttgcatccgacttgtggtctcgctgttccttgggagggtctcctctgagtgattgactac |
| | ccgtcagcgggggtctttcacacatgcagcatgtatcaaaattaatttggttttttttct |
| | taagctgtgccttctagttgccagccatctgttgtttgcccctcccccgtgccttccttg |
| | accctggaaggtgccactcccactgtcctttcctaataaaatgaggaaattgcatcgcat |
| | tgtctgagtaggtgtcattctattctggggggtggggggggcaggacagcaagggggagg |
| | attgggaagacaatagcaggcatgctggggatgcggtgggctctatggagatcccgcggt |
| | acctcgcgaatgcatctagatccaatggcctttttggcccagacatgataagatacattg |
| | atgagtttggacaaaccacaactagaatgcagtgaaaaaaatgctttatttgtgaaattt |
| | gtgatgctattgctttatttgtaaccattataagctgcaataaacaagttgcggccgctt |
| | agccctcccacacataaccagagggcagcaattcacgaatcccaactgccgtcggctgtc |
| | catcactgtccttcactatggctttgatcccaggatgcagatcgagaagcacctgtcggc |
| | accgtccgcaggggctcaagatgcccctgttctcatttccgatcgcgacgatacaagtca |
| | ggttgccagctgccgcagcagcagcagtgcccagcaccacgagttctgcacaaggtcccc |
| | cagtaaaatgatatacattgacaccagtgaagatgcggccgtcgctagagagagctgcgc |
| | tggcgacgctgtagtcttcagagatggggatgctgttgattgtagccgttgctctttcaa |
| | tgagggtggattcttcttgagacaaaggcttggccatgcggccgccgctcggtgttcgag |
| | gccacacgcgtcaccttaatatgcgaagtggacctcggaccgcgccgccccgactgcatc |
| | tgcgtgttcgaattcgccaatgacaagacgctgggggggtttgtgtcatcatagaactaa |
| | agacatgcaaatatatttcttccggggggtaccggcctttttggccATTGGatcggatct |
| | ggccaaaaaggcccttaagtatttacattaaatggccatagtacttaaagttacattggc |
| | ttccttgaaataaacatggagtattcagaatgtgtcataaatatttctaattttaagata |
| | gtatctccattggctttctactttttcttttatttttttttgtcctctgtcttccatttg |
| | ttgttgttgttgtttgtttgtttgtttgttggttggttggttaatttttttttaaagatc |
| | ctacactatagttcaagctagactattagctactctgtaacccagggtgaccttgaagtc |
| | atgggtagcctgctgttttagccttcccacatctaagattacaggtatgagctatcattt |
| | ttggtatattgattgattgattgattgatgtgtgtgtgtgtgattgtgtttgtgtgtgtg |
| | aTtgtgTaTatgtgtgtatggTtgtgtgtgaTtgtgtgtatgtatgTTtgtgtgtgaTtg |
| | TgtgtgtgtgaTtgtgcatgtgtgtgtgtgtgaTtgtgtTtatgtgtatgaTtgtgtgtg |
| | tgtgtgtgtgtgtgtgtgtgtgtgtgtgtgtgtgtgtgttgtgTaTaTatatttatggta |
| | gtgagagGcaacgctccggctcaggtgtcaggttggtttttgagacagagtctttcactt |
| | agcttggaattcactggccgtcgttttacaacgtcgtgactgggaaaaccctggcgttac |
| | ccaacttaatcgccttgcagcacatccccctttcgccagctggcgtaatagcgaagaggc |
| | ccgcaccgatcgcccttcccaacagttgcgcagcctgaatggcgaatggcgcctgatgcg |
| | gtattttctccttacgcatctgtgcggtatttcacaccgcatatggtgcactctcagtac |
| | aatctgctctgatgccgcatagttaagccagccccgacacccgccaacacccgctgacgc |
| | gccctgacgggcttgtctgctcccggcatccgcttacagacaagctgtgaccgtctccgg |
| | gagctgcatgtgtcagaggttttcaccgtcatcaccgaaacgcgcgagacgaaagggcct |
| | cgtgatacgcctatttttataggttaatgtcatgataataatggtttcttagacgtcagg |
| | tggcacttttcggggaaatgtgcgcggaacccctatttgtttatttttctaaatacattc |
| | aaatatgtatccgctcatgagacaataaccctgataaatgcttcaataatattgaaaaag |
| | gaagagtatgagtattcaacatttccgtgtcgcccttattcccttttttgcggcattttg |
| | ccttcctgtttttgctcacccagaaacgctggtgaaagtaaaagatgctgaagatcagtt |
| | gggtgcacgagtgggttacatcgaactggatctcaacagcggtaagatccttgagagttt |
| | tcgccccgaagaacgttttccaatgatgagcacttttaaagttctgctatgtggcgcggt |
| | attatcccgtattgacgccgggcaagagcaactcggtcgccgcatacactattctcagaa |
| | tgacttggttgagtactcaccagtcacagaaaagcatcttacggatggcatgacagtaag |
| | agaattatgcagtgctgccataaccatgagtgataacactgcggccaacttacttctgac |
| | aacgatcggaggaccgaaggagctaaccgcttttttgcacaacatgggggatcatgtaac |
| | tcgccttgatcgttgggaaccggagctgaatgaagccataccaaacgacgagcgtgacac |
| | cacgatgcctgtagcaatggcaacaacgttgcgcaaactattaactggcgaactacttac |
| | tctagcttcccggcaacaattaatagactggatggaggcggataaagttgcaggaccact |
| | tctgcgctcggcccttccggctggctggtttattgctgataaatctggagccggtgagcg |
| | tgggtctcgcggtatcattgcagcactggggccagatggtaagccctcccgtatcgtagt |
| | tatctacacgacggggagtcaggcaactatggatgaacgaaatagacagatcgctgagat |
| | aggtgcctcactgattaagcattggtaactgtcagaccaagtttactcatatatacttta |
| | gattgatttaaaacttcatttttaatttaaaaggatctaggtgaagatcctttttgataa |
| | tctcatgaccaaaatcccttaacgtgagttttcgttccactgagcgtcagaccccgtaga |
| | aaagatcaaaggatcttcttgagatcctttttttctgcgcgtaatctgctgcttgcaaac |
| | aaaaaaaccaccgctaccagcggtggtttgtttgccggatcaagagctaccaactctttt |
| | tccgaaggtaactggcttcagcagagcgcagataccaaatactgtccttctagtgtagcc |
| | gtagttaggccaccacttcaagaactctgtagcaccgcctacatacctcgctctgctaat |
| | cctgttaccagtggctgctgccagtggcgataagtcgtgtcttaccgggttggactcaag |
| | acgatagttaccggataaggcgcagcggtcgggctgaacggggggttcgtgcacacagcc |
| | cagcttggagcgaacgacctacaccgaactgagatacctacagcgtgagctatgagaaag |
| | cgccacgcttcccgaagggagaaaggcggacaggtatccggtaagcggcagggtcggaac |
| | aggagagcgcacgagggagcttccagggggaaacgcctggtatctttatagtcctgtcgg |
| | gtttcgccacctctgacttgagcgtcgatttttgtgatgctcgtcaggggggcggagcct |
| | atggaaaaacgccagcaacgcggcctttttacggttcctggccttttgctggccttttgc |
| | tcacatgttctttcctgcgttatcccctgattctgtggataaccgtattaccgcctttga |
| | gtgagctgataccgctcgccgcagccgaacgaccgagcgcagcgagtcagtgagcgagga |
| | agcggaagagcgcccaatacgcaaaccgcctctccccgcgcgttggccgattcattaatg |
| | cagctggcacgacaggtttcccgactggaaagcgggcagtgagcgcaacgcaattaatgt |
| | gagttagctcactcattaggcaccccaggctttacactttatgcttccggctcgtatgtt |
| | gtgtggaattgtgagcggataacaatttcacacaggaaacagctatgaccatgattacgc |
| | c (SEQ ID NO: 316) |

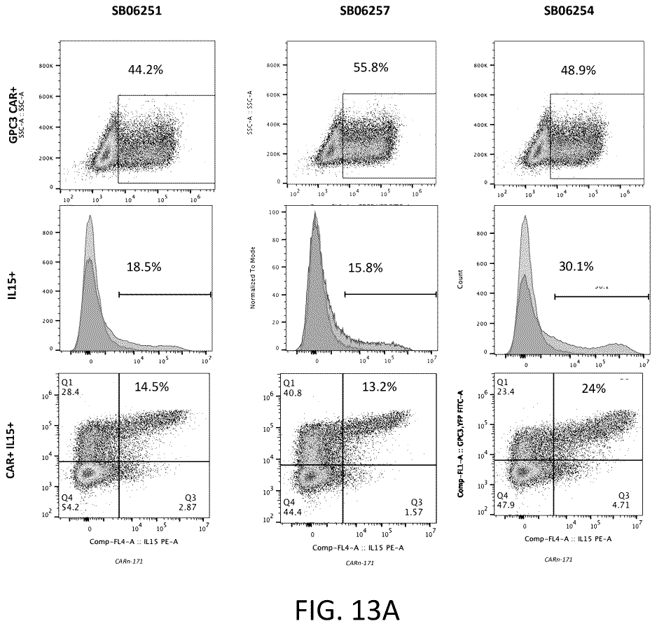

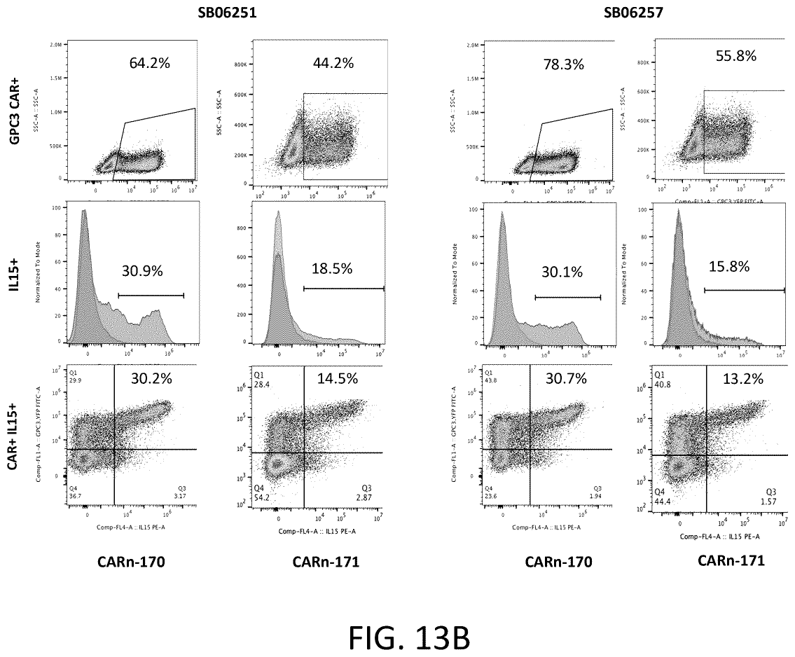

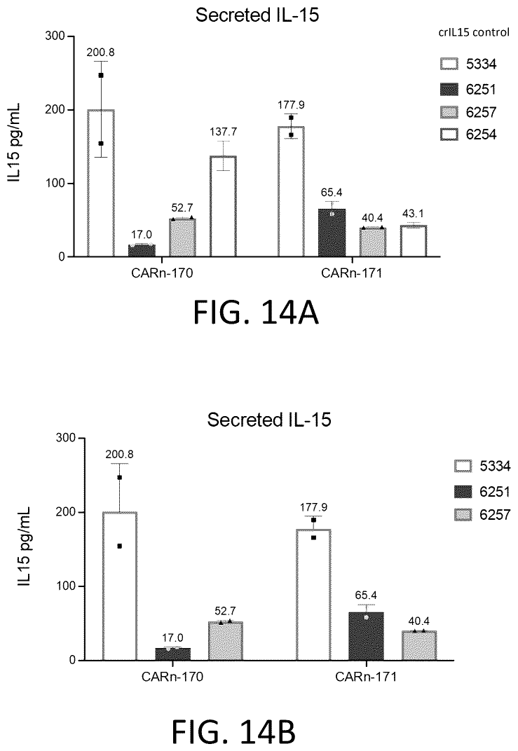

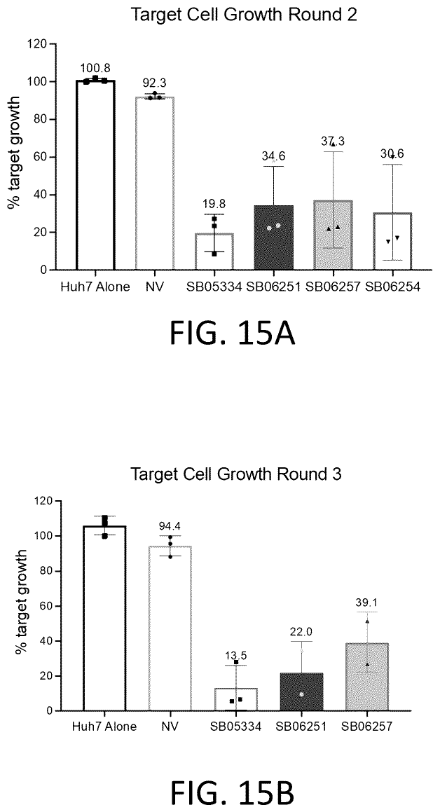

NK cells comprising CARs comprising OX40 transmembrane (TM) and co-stimulatory (co-stim) domains, SB06251, SB06257, and SB06254, were assessed for expression of constructs as described above. Results as determined by flow cytometry are shown in FIG. 13A and FIG. 13B. Secreted IL15 was measured as described above; results are summarized in FIG. 14A and FIG. 14B. To assess killing of the target cell population, cell growth was determined as described above (FIG. 15A and FIG. 15B).

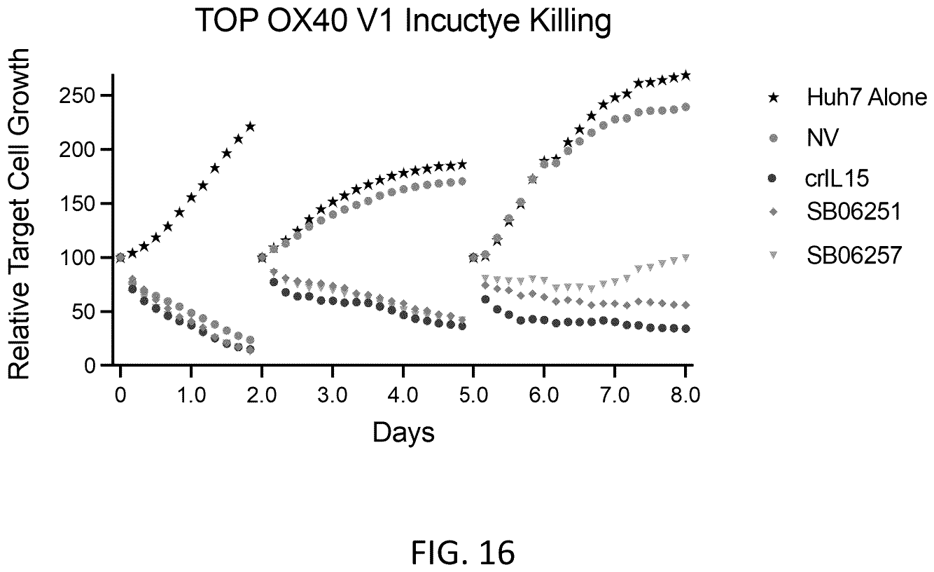

Serial killing by the NK cells comprising SB06257 was also assessed. Target cells were added at Days 0, 2, and 5, and Huh7 target cell count was calculated using an Incucyte. Results are shown in FIG. 16.

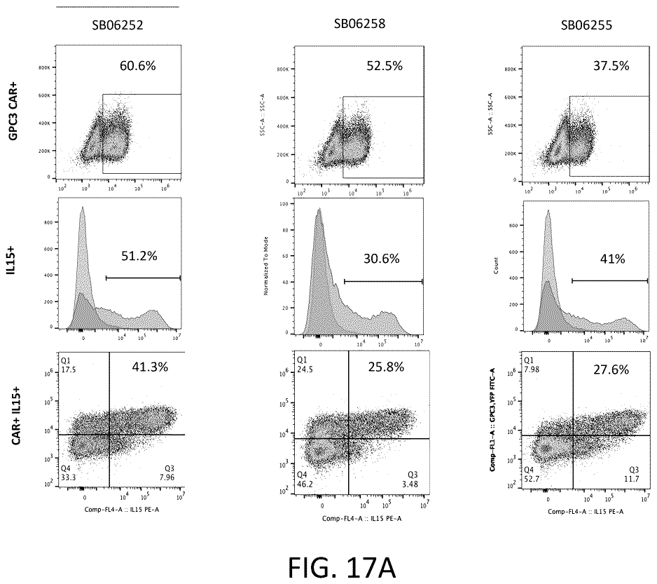

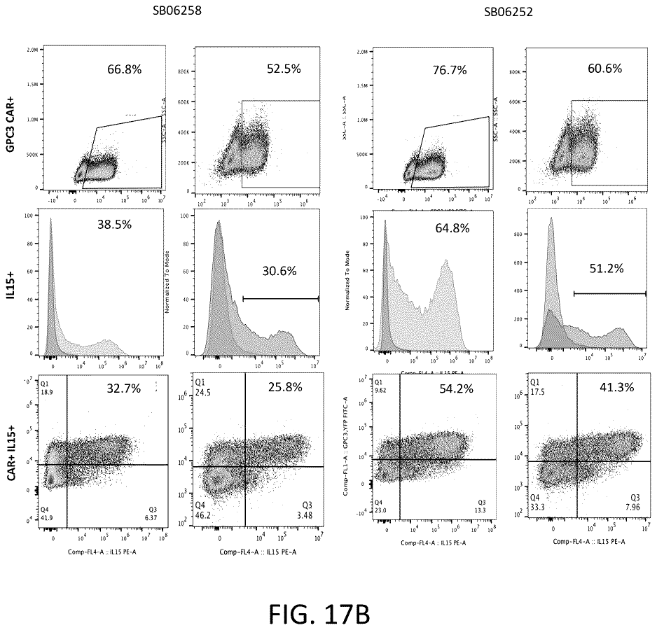

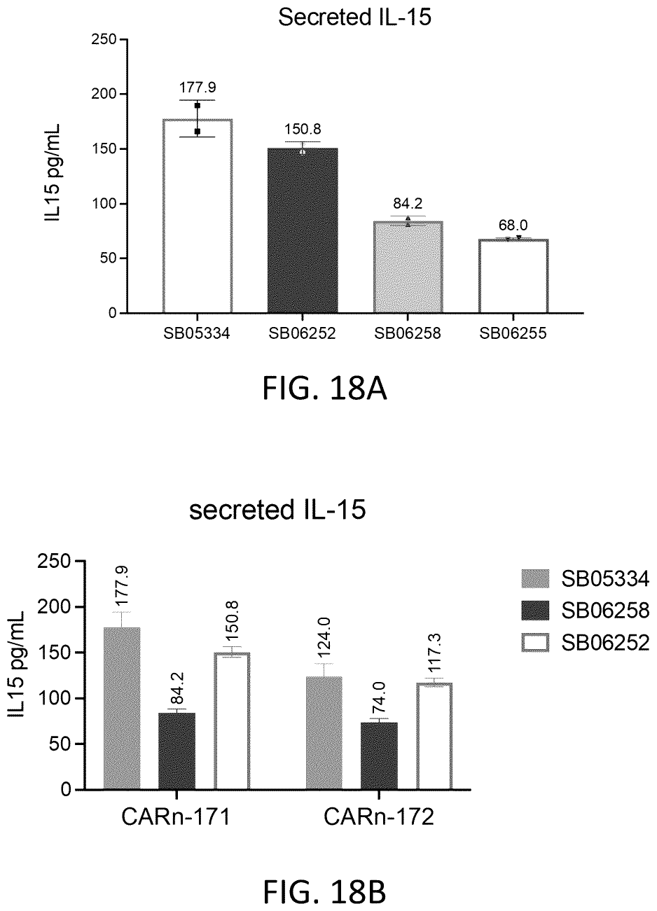

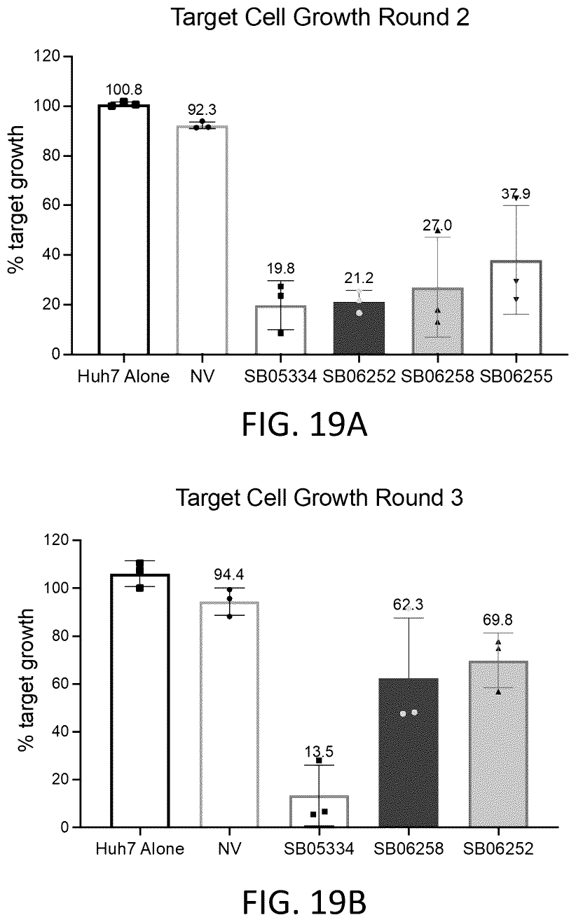

NK cells comprising CARs comprising CD28 co-stimulatory (co-stim) domains, SB06252, SB06258, and SB06255, were assessed for expression of constructs as described above. Results as determined by flow cytometry FACS are shown in FIG. 17A and FIG. 17B. Secreted IL15 was measured as described above; results are summarized in FIG. 18A and FIG. 18B. To assess killing of the target cell population, cell growth was determined as described above (FIG. 19A and FIG. 19B).

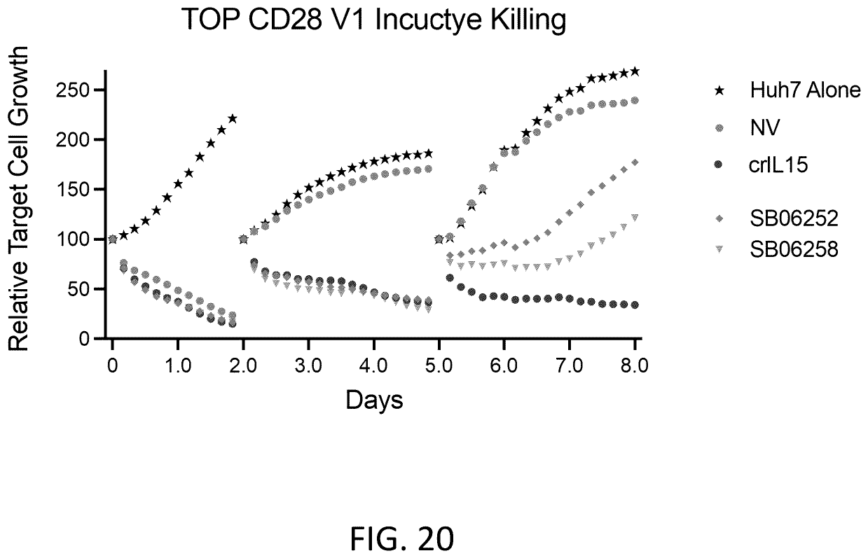

Serial killing by the NK cells comprising SB06252 and SB06258 was also assessed. Target cells were added at Days 0, 2, and 5, and Huh7 target cell count was calculated using an Incucyte. Results are shown in FIG. 20.

### Screening for Bicistronic Constructs

0.5e6 NK donor 7B cells were expanded in the presence of fresh irradiated mbIL21/IL15K562 feeder cells on retronectin coated non-TC 24-well plates. Spinoculation was performed at 800 g at 32° C. for 2 hr. For viral transduction, 300 μl of virus added, for a total transduction volume of 500 μl.

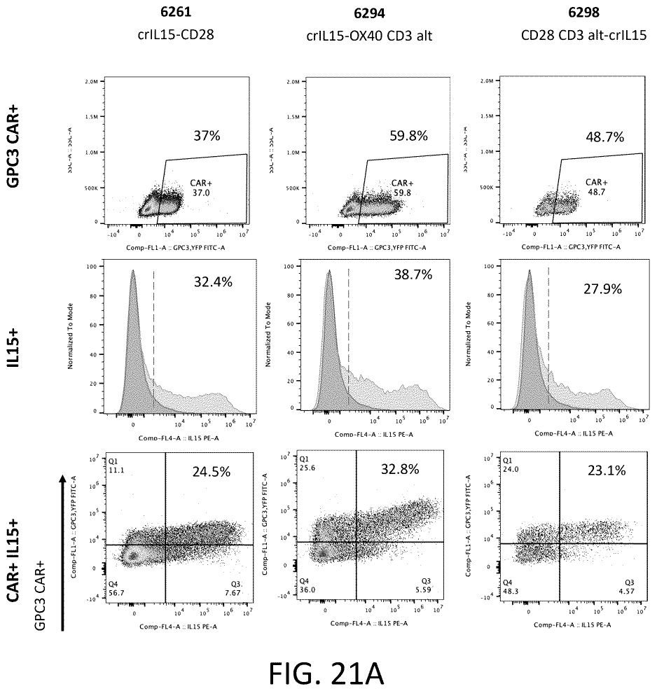

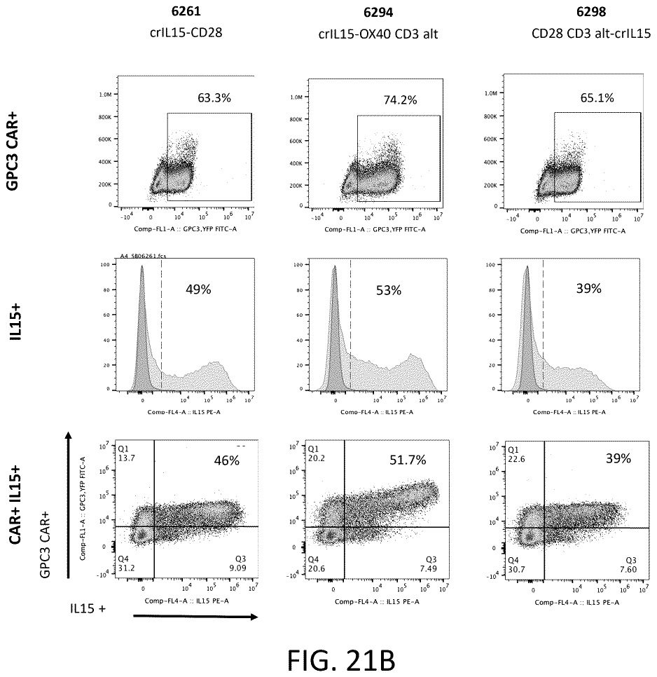

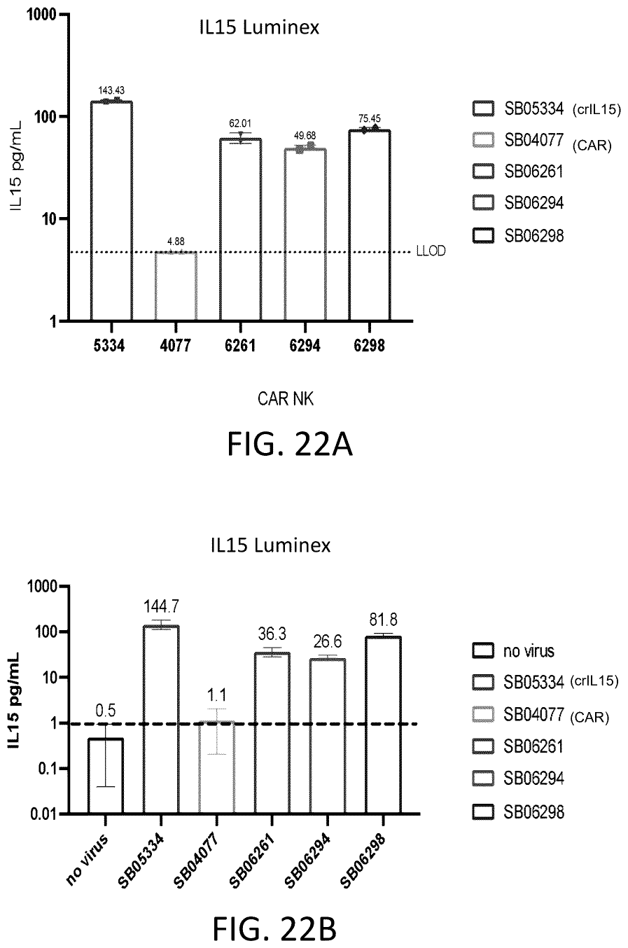

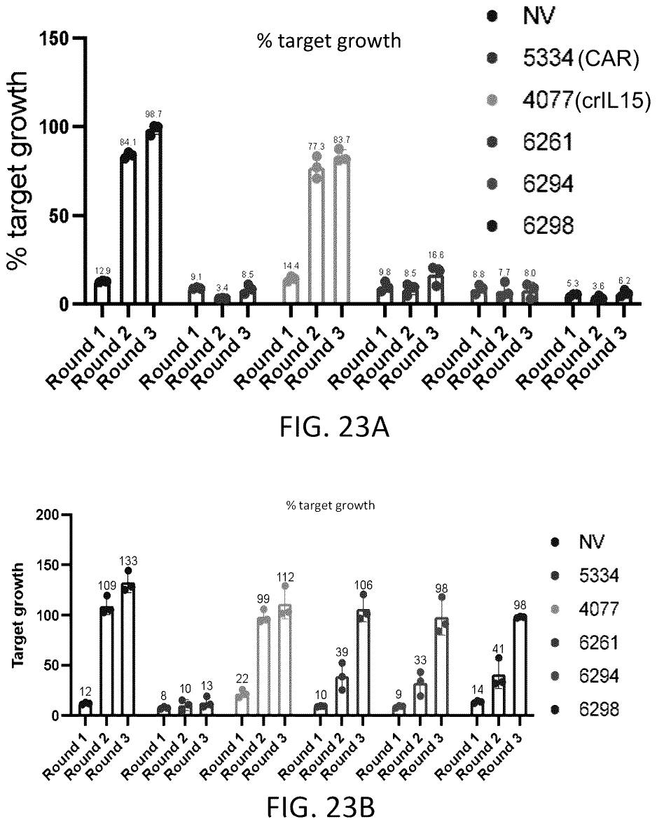

Cells were cultured in the same plate for the entire expansion period, in 2 ml final volume. Three partial media exchanges were performed as described above before assessing expression and using the cells in functional assays. Results of expression and cytotoxicity against target cells are shown in Table 8. As shown, SB06261, SB6294, and SB6298 showed good CAR and IL15 expression levels as determined by flow and good cytotoxicity in serial killing assay (n=2). Flow cytometry expression data is shown in FIG. 21A and FIG. 21B, IL15 levels are shown in FIG. 22A and FIG. 22B, and cell growth of the target cell population (as a measure of cell killing by the NK cells) is shown in FIG. 23A and FIG. 23B.

Due to its high CAR and IL15 expression and performance in functional assays, SB06294, a retroviral vector with crIL15 2A OX40 CAR design, was selected for further study.

TABLE 8

| | | | | | | | | Double + | sIL15 | Round | Round | Round |
|---|---|---|---|---|---|---|---|---|---|---|---|---|
| SB# | Virus | Insert 1 | 2A | Insert 2 | Expt # | CAR % | mbIL15% | % | (pg/mL) | 1 | 2 | 3 |
| 6261 | Sinvec | crIL15 | E2A | CD28 | CARn-173 | 37 | 32 | 24.5 | 62 | 9.8 | 8.5 | 16.6 |
| | | TACE-10 | T2A | | CARn-174 | 63.3 | 49 | 46 | 36 | 9.5 | 39 | 100 |
| 6294 | Retrovec | crIL15 | E2A | OX40- | CARn-173 | 59.8 | 38.7 | 32.8 | 50 | 8.8 | 7.7 | 8 |
| | | TACE-10 | T2A | CD3 alt | CARn-174 | 74 | 53 | 52 | 27 | 9.2 | 32 | 98 |
| 6298 | Sinvec | CD28-CD3 | E2A | crIL15 | CARn-173 | 48.7 | 27.9 | 23.1 | 75 | 5.3 | 3.6 | 6.2 |
| | | alt | T2A | TACE-10 | CARn-174 | 65 | 39 | 39 | 82 | 13.8 | 41 | 98 |

### Analysis of TACE-OPT Constructs

Bicistronic TACE-OPT constructs comprising a TACE10 cleavage site, were analyzed for CAR and IL15 expression, CNA assay, and payload assay for secreted cytokines, as described above. A TACE10 cleavage site was modified to increase cleavage kinetics, resulting in “TACE-OPT,” which results in higher cytokine secretion levels as compared to the parent TACE10. Tricistronic constructs were analyzed for CAR and IL15 expression, and IL12 induction.

Briefly, 0.5e6 NK donor 7B cells were expanded in the presence of fresh irradiated mbIL21/IL15K562 feeder cells on retronectin coated non-TC 24-well plates. Spinoculation was performed at 800 g at 32° C. for 2 hr. For viral transduction, 300 μl of virus was added, for a total transduction volume of 500 μl.

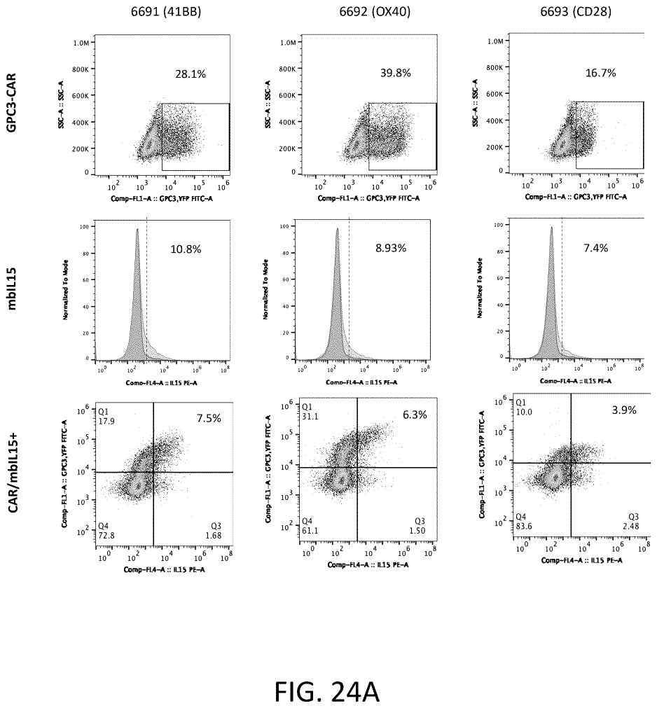

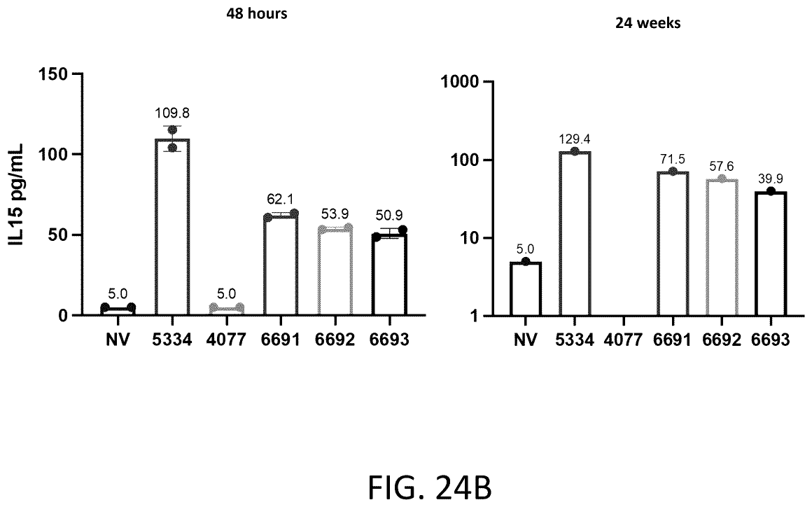

Bicistronic constructs SB6691 (comprising 41BB co-stimulatory domain), SB6692 (comprising OX40 co-stimulatory domain), and SB6693 (comprising CD28 co-stimulatory domain) were assessed by flow cytometry for expression of CAR and IL15 (FIG. 24A). Copy number of each construct per cell is shown in Table 9. IL15 secretion was quantified as described above at 48 hours and 24 weeks post-tranduction (FIG. 24B). While the TACE-OPT constructs tested have similar expression levels and cytokine secretion, SB06692 (comprising an OX40 co-stimulatory domain) has the highest CAR expression.

TABLE 9

| | YP7 [CAR] | IL15 | WPRE |
|---|---|---|---|
| COPY # | (copies/cell) | (copies/cell) | (copies/cell) |
| SB06691 | 116.6 | 120.2 | 147.2 |
| SB06692 | 308.3 | 318.3 | 313.0 |
| SB06693 | 48.8 | 49.4 | 57.6 |

SB06258, SB06257, SB06294 and SB06692 demonstrated high CAR expression, high crIL15 expression (both membrane-bound and secreted), and high serial killing function in vitro.

### Example 4: Expression of IL12 from Bidirectional Constructs Encoding a Regulatable, Cleavable-Release IL12 and a Synthetic Transcription Factor

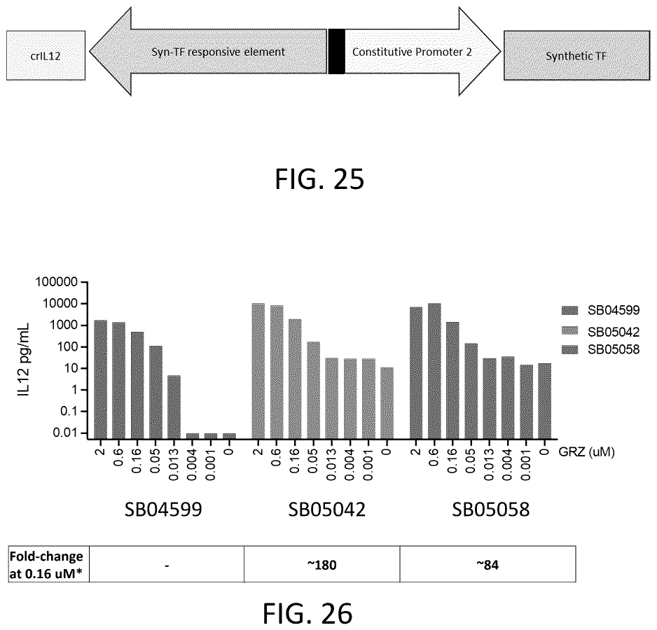

IL12 expression was assessed for NK cells transduced with bidirectional constructs encoding regulatable, cleavable release IL12 and a synthetic transcription factor, with transductions performed as described in Example 3 above. The regulatable, cleavable IL12 is operably linked to a synthetic transcription factor-responsive promoter, which includes a ZF-10-1 binding site and a minimal promoter sequence. The synthetic transcription factor includes a DNA binding domain and a transcriptional activation domain. Between the DNA binding domain and the transcriptional activation domain is a protease domain that is regulatable by a protease inhibitor and cognate cleavage site for the protease. In the absence of an inhibitor of the protease, the protease induces cleavage at the cleavage site, resulting in a non-functional synthetic transcription factor. In the presence of the protease inhibitor, the synthetic transcription factor is not cleaved and is thus capable of modulating expression of the cleavable IL12. The expression cassette encoding the cleavable release IL12 includes a chimeric polypeptide including the IL12 and a transmembrane domain. Between the IL12 and the transmembrane domain is a protease cleavage domain that is cleavable by a protease endogenous to NK cells. A cartoon diagram of the bidirectional constructs encoding cleavable release 12 is shown in FIG. 25. Parameters of the constructs tested herein are summarized in Table 10. Designs tested include: cleavable-release IL12 (crIL12) regulated constructs (32 constructs tested), soluble IL12 (sIL12) regulated and/or WPRE and polyA+different destabilizing domains (32 constructs tested), destabilizing domain and/or WPRE and polyA (26 constructs tested). Initial studies demonstrated toxicity generally due to leaky expression of IL12, resulting in poor NK cell viability and expansion following transduction (data not shown). A screen was designed to discovere constructs that could overcome or reduce IL12 associated toxicity by modifying the parameters in Table 10. A summary of screening criteria for is shown in Table 11A. Suitable candidates SB05058 and SB05042 (both gammaretroviral vectors) and SB04599 (lentiviral vector) were identified. A summary of these candidates is provided in Table 11B.

TABLE 10
Parameters tested

| | ZF | | Effector domain | |
|---|---|---|---|---|
| Promoter | copies | IL12 | orientation | Modifications |
| SFFV | 10-1 | crIL12 | N-terminal | UTR |
| | | CD16 | | |
| SV40 | 5-7 | crIL12 | C-terminal | Destabilizing |
| | | TACE 10 | | domains |
| | | SIL12 | | Remove |
| | | | | WPRE/PolyA |

TABLE 11A
Screen

| Metrics | Recommendations |
|---|---|
| IL12 induction at 0.1 uM GRZ in 24 hours in vitro | >50-fold |
| | >1000 pg/ml |
| NK cell viability Day 10 post-transduction | >75% |
| Fold-expansion in 10 days (mid-scale, 6 well G- | >10-fold |
| rex) | (research) |

TABLE 11B
Candidates

| | | | | | Effector |
|---|---|---|---|---|---|
| Viral | | | | NS3 | domain |
| vector | SB# | ZF | IL12 | promoter | orientation |
| Gamma retro | SB05058 | 5-7 | CD16 crIL12 | SV40 | C-terminal |
| Gamma retro | SB05042 | 5-7 | CD16 crIL12 | SV40 | N-terminal |
| Lenti | SB04599 | 10-1 | 1X SLDE | SFFV | C-terminal |
| | | | sIL12 | | |

Assessment of gammaretroviral vectors and lentiviral vectors was performed. A grazoprevir (GRZ) dose response assay measuring IL12 secretion demonstrated that both gammaretroviral constructs showed higher sensitivity to GRZ as compared to the lentiviral construct (FIG. 26 and Table 12A).

TABLE 12A

| [GRZ] | | | |
|---|---|---|---|
| μM | SB04599 | SB05042 | SB05058 |
| 2 | 1762.68 | 10629.99 | 7167.37 |
| 0.6 | 1387.37 | 8722.87 | 10922.93 |
| 0.16 | 514.02 | 2031.82 | 1470.22 |
| 0.05 | 112.14 | 173.44 | 151.69 |
| 0.013 | 4.80 | 31.57 | 29.72 |
| 0.004 | u.d | 28.48 | 35.83 |
| 0.001 | u.d | 28.48 | 14.83 |
| 0 | u.d | 11.27 | 17.56 |

u.d = <5 pg/ml, undetectable

Construct expression and cellular viability were determined 10-days following transduction of NK cells. Results are shown in Table 12B and demonstrate an above 10-fold cellular expansion in mid-scale plates, above 85% viability, and greater than 2 copies/cell. Gammaretroviral vectors displayed higher transduction efficiency of NK cells than lentiviral vectors, particularly for the bidirectional vectors tested.

TABLE 12B

| | | | Viability | Fold | CNA (avg |
|---|---|---|---|---|---|
| Viral vector | SB# | MOI | (%) | expansion | copies/cell) |
| | NV | n/a | 88 | 29.9 | n/a |
| Lenti | 4599 | 29.7 | 89 | 19.6 | 1.0 |
| Gamma | 5042 | 83.5 | 89 | 15.1 | 1.6 |
| retro | | | | | |
| Gamma | 5058 | 0.8 | 86 | 11.6 | 1.8 |
| retro | | | | | |

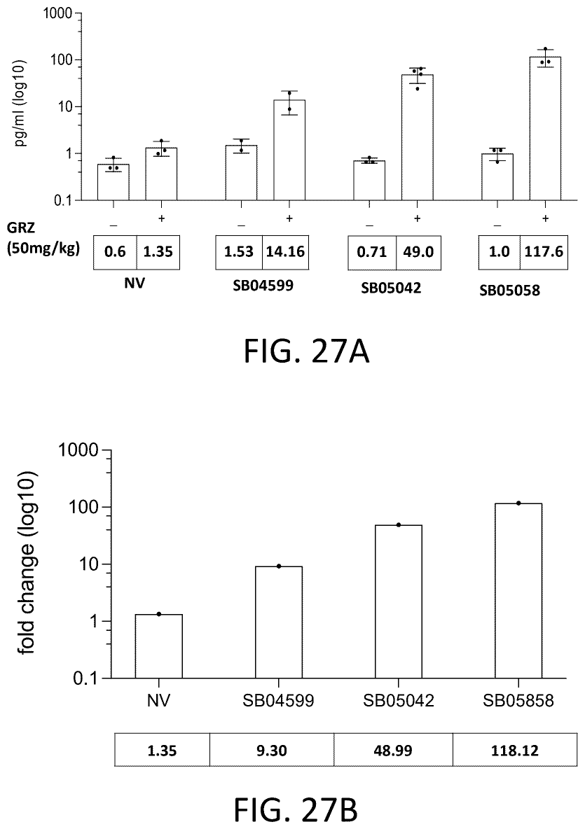

Additionally, IL12 induction was assessed in vivo. Briefly, mice were injected intravenously with transduced NK cells at a dose of 15e6 cells in a 200 μL volume. Blood was collected 24 hours after injection and assayed for IL12 expression levels. SB05042 and SB05058 showed the highest IL12 fold-induction. No induction was observed in 10 mg/kg dose groups (data not shown). The percentage of % hNKs in mouse blood was determined to be less than 2% for all constructs. Results are summarized in Table 12C. IL12 levels are shown in FIG. 27A and fold change is shown in FIG. 27B.

TABLE 12C

| Viral | | −GRZ | +GRZ | Fold- |
|---|---|---|---|---|
| vector | SB# | (pg/ml) | (pg/ml) | change |
| | NV | 0.6 | 1.35 | 1.35 |
| Lenti | 4599 | 1.35 | 14.16 | 9.30 |
| Gamma | 5042 | 0.71 | 49.0 | 48.99 |
| retro | | | | |
| Gamma | 5058 | 1.0 | 117.62 | 118.12 |
| retro | | | | |

The gammaretroviral vectors (SB05042 and SB305058) demonstrated superior IL12 induction in vitro compared to the lentiviral vector (SB04599), while maintaining good viability and cell growth post-transduction. Importantly, both gammaretroviral vectors tested showed IL12 induction in NK cells in vivo.

Full-length sequences of constructs described in this Example are shown in Table 13.

TABLE 13

| Construct | Full nucleotide sequence |
|---|---|
| SB05042 | aagcttggaattcgagcttgcatgcctgcaggtcgttacataacttacggtaaatggcccgcctggctgaccgcccaacgaccccc |
| (B7-1(TM)-CD16 | gcccattgacgtcaataatgacgtatgttcccatagtaacgccaatagggactttccattgacgtcaatgggtggagtatttacggtaa |
| TACE(cleavage | actgcccacttggcagtacatcaagtgtatcatatgccaagtacgccccctattgacgtcaatgacggtaaatggcccgcctggcatt |
| site)-IL12- | atgcccagtacatgaccttatgggactttcctacttggcagtacatctacgtattagtcatcgctattaccatggtgatgcggttttggca |
| YB_TATA ZFBD | gtacatcaatgggcgtggatagcggtttgactcacggggatttccaagtctccaccccattgacgtcaatgggagtttgttttggcac |
| (syn prmoter)-A2 | caaaatcaacgggactttccaaaatgtcgtaacaactccgccccattgacgcaaatgggcggtaggcgtgtacggtgggaggtct |
| (insulator)-SV40 | atataagcagagctcaataaaagagcccacaacccctcactcggcgcgccagtcctccgattgactgagtcgcccgggtacccgt |
| (promoter)-Syn | gtatccaataaaccctcttgcagttgcatccgacttgtggtctcgctgttccttgggagggtctcctctgagtgattgactacccgtcag |
| TF (NLS + | cgggggtctttcatttgggggctcgtccgagatcgggagacccctgcccagggaccaccgacccaccaccgggaggtaagctg |
| mini VPR | gccagcaacttatctgtgtctgtccgattgtctagtgtctatgactgattttatgcgcctgcgtcggtactagttagctaactagctctgta |
| activation | tctggcggacccgtggtggaactgacgagttcggaacacccggccgcaaccctgggagacgtcccagggacttcgggggccgt |
| domain + NS3 | ttttgtggcccgacctgagtcctaaaatcccgatcgtttaggactctttggtgcaccccccttagaggagggatatgtggttctggtag |
| protease + | gagacgagaacctaaaacagttcccgcctccgtctgaatttttgctttcggtttgggaccgaagccgcgccgcgcgtcttgtctgctg |
| ZFBD DNA | cagcatcgttctgtgttgtctctgtctgactgtgtttctgtatttgtctgaaaatatgggccccccctcgagtccccagcatgcctgctatt |
| binding domain) | ctcttcccaatcctcccccttgctgtcctgccccaccccaccccccagaatagaatgacacctactcagacaatgcgatgcaatttcc |
| | tcattttattaggaaaggacagtgggagtggcaccttccagggtcaaggaaggcacgggggaggggcaaacaacagatggctg |
| | gcaactagaaggcacagttacttaCACAGGCCGCACAGATTCTCTCCGCAGCCGTTCGTTTCT |
| | TCTCCGCTCTCTGCACCGAGGGGCGAAGCAGTAGGTCAGGCAACAGATCACGA |
| | AGATGCCGTTCACGGAGATCAGTGTGATGGCCCAGCTTGGCAGCAGTTGAAGA |
| | GATCCACCGCCACTGCCACCGCCGCCACTACCGCCACCAAAGAAGCTGGAGAT |
| | TGTAGACACGGCGAGTCCCTGTGTAATTCCAGATCCTCCGCCTCCGCTACCACC |
| | TCCGCCGCTAGAGGCGTTCAGGTAGCTCATCACTCTGTCGATGGTCACGGCTCT |
| | GATCCGGAAGGCGTGCAGCAGGATGCACAGCTTGATCTTGGTCTTGTAGAAGT |
| | CGGGTTCTTCCAGGCTAGACTTCTGGGGCACTGTCTCGCTGTTGAAGTTCAGGG |
| | CCTGCATCAGCTCGTCGATCACGGCCAGCATATTCTGGTCCAGGAAGATCTGC |
| | CGCTTGGGGTCCATCAGCAGCTTGGCGTTCATGGTCTTGAATTCCACCTGGTAC |
| | ATCTTCAGGTCCTCGTAGATGCTGCTCAGGCACAGGGCCATCATGAAGGAGGT |
| | CTTTCTGCTGGCCAGGCAAGAGCCGTTGGTGATGAAGCTGGTTTCCCGGCTGTT |
| | CAGGCAGCTCTCGTTCTTGGTCAGTTCCAGAGGCAGGCAGGCTTCCACGGTGC |
| | TGGTCTTATCCTTGGTGATGTCCTCGTGGTCGATTTCCTCGCTGGTGCAGGGGT |
| | AGAATTCCAGGGTCTGTCTGGCCTTCTGCAGCATGTTGGACACGGCTCTCAGCA |
| | GGTTCTGGCTGTGGTGCAGACAAGGGAACATGCCAGGATCAGGAGTGGCCAC |
| | AGGCAGGTTTCTAGATCCGCCGCCAGATCCACCACCTGATCCGCCACCGCTTCC |
| | TCCGCCAGAACATGGCACGCTGGCCCATTCGCTCCAAGAGCTGCTGTAGTACC |
| | GGTCCTGGGCTCTGACGCTGATGCTGGCGTTCTTTCTGCAGATCACGGTGGCGC |
| | TGGTCTTGTCGGTGAACACCCGGTCCTTTTTCTCGCGCTTGGACTTGCCCTGCA |
| | CTTGCACGCAAAAGGTCAGGCTGAAGTAGCTGTGGGGTGTAGACCAGGTGTCG |
| | GGGTACTCCCAGGACACTTCCACCTGTCTGCTGTTCTTCAGAGGCTTCAGCTGC |
| | AGGTTCTTTGGAGGATCGGGCTTGATGATGTCCCGGATGAAAAAGCTGGAGGT |
| | GTAGTTCTCGTACTTCAGCTTGTGCACGGCGTCCACCATCACTTCGATAGGCAG |
| | AGACTCTTCGGCGGCTGGACAGGCGCTGTCCTCTTGGCATTCCACGCTGTACTC |
| | GTATTCTTTGTTGTCGCCCCGCACTCTTTCGGCAGACAGTGTAGCGGCGCCACA |
| | TGTAACGCCCTGAGGATCACTGCTGCCTCTGCTGGACTTCACGCTGAAGGTCA |
| | GGTCGGTGCTGATGGTGGTCAGCCACCAACATGTGAACCGGCCGCTGTAGTTC |
| | TTGGCCTCGCATCTCAGGAAGGTCTTGTTCTTGGGCTCTTTCTGGTCCTTCAGG |
| | ATGTCGGTGCTCCAAATGCCATCCTCTTTCTTGTGGAGCAGCAGCAGGCTGTGG |
| | CTCAGCACTTCTCCGCCTTTGTGACAGGTGTACTGGCCGGCGTCGCCAAACTCT |
| | TTCACTTGGATGGTCAGGGTCTTGCCGCTGCCGAGCACCTCGCTAGACTGATCC |
| | AGTGTCCAGGTGATGCCGTCCTCTTCAGGGGTATCGCAGGTCAGCACCACCAT |
| | CTCGCCAGGAGCATCGGGATACCAGTCCAGTTCCACCACGTACACGTCTTTCTT |
| | CAGCTCCCAGATGGCCACCAGAGGAGAGGCCAGGAACACCAGGCTGAACCAG |
| | CTGATGACCAGCTGCTGGTGACACATCATGGTGGCGACACCGGTACGCGTTGG |
| | CCCCCATTATATACCCTCTAGAACTAGTtatccactccgtgtaagggagagtgagcctcttacgaatcT |
| | TCGGCGTCGACTGCTTCattccgaggcgactgataccTTCGGCGTCGACTGCTTCatacgaaggc |
| | agtccgattcTTCGGCGTCGACTGCTTCaacctttactgagacgggacTTCGGCGTCGACTGCTTC |
| | aaaggcgttgcgaatcctcatgcgattgttacgaaacccgTTAATTAAAGAGCGAGATTCCGTCTCAAA |
| | GAAAAAAAAAGTAATGAAATGAATAAAATGAGTCCTAGAGCCAGTAAATGTC |
| | GTAAATGTCTCAGCTAGTCAGGTAGTAAAAGGTCTCAACTAGGCAGTGGCAGA |
| | GCAGGATTCAAATTCAGGGCTGTTGTGATGCCTCCGCAGACTCTGAGCGCCAC |
| | CTGGTGGTAATTTGTCTGTGCCTCTTCTGACGTGGAAGAACAGCAACTAACAC |
| | ACTAACACGGCATTTACTATGGGCCAGCCATTGTCCATCTAGATGGccgataaaataa |
| | aagattttatttagtctccagaaaaaggggggaatgaaagaccccacctgtaggtttggcaagctagctgcaGTGTGTCAG |
| | TTAGGGTGTGGAAAGTCCCCAGGCTCCCCAGCAGGCAGAAGTATGCAAAGCAT |
| | GCATCTCAATTAGTCAGCAACCAGGTGTGGAAAGTCCCCAGGCTCCCCAGCAG |
| | GCAGAAGTATGCAAAGCATGCATCTCAATTAGTCAGCAACCATAGTCCCGCCC |
| | CTAACTCCGCCCATCCCGCCCCTAACTCCGCCCAGTTCCGCCCATTCTCCGCCC |
| | CATGGCTGACTAATTTTTTTTATTTATGCAGAGGCCGAGGCCGCCTCTGCCTCT |
| | GAGCTATTCCAGAAGTAGTGAGGAGGCTTTTTTGGAGGCCTAGGCTTTTGCAA |
| | AGGATCCGCCACCATGCCCAAGAAAAAGCGGAAGGTGGACGCCCTGGACGAC |
| | TTCGATCTGGATATGCTGGGCAGCGACGCTCTGGATGATTTTGACCTGGACATG |
| | CTCGGCTCTGATGCACTCGACGATTTCGACCTCGATATGTTGGGATCTGATGCC |
| | CTTGATGACTTTGATCTCGACATGTTGATCAATAGCCGGTCCAGCGGCAGCCCC |
| | AAGAAGAAGAGAAAAGTCGGCTCTGGCGGCGGATCTGGCGGTTCTGGATCTGT |
| | TTTGCCCCAAGCTCCTGCTCCTGCACCAGCTCCAGCTATGGTTTCTGCTCTGGC |
| | TCAGGCTCCAGCTCCTGTGCCTGTTCTTGCTCCTGGACCTCCTCAGGCTGTTGCT |
| | CCACCAGCACCTAAACCTACACAGGCCGGCGAGGGAACACTGTCTGAAGCTCT |
| | GCTGCAGCTCCAGTTCGACGACGAAGATCTGGGAGCCCTGCTGGGCAATAGCA |
| | CAGATCCTGCCGTGTTCACCGATCTGGCCAGCGTGGACAATAGCGAGTTCCAG |
| | CAGCTCCTGAACCAGGGCATTCCTGTGGCTCCTCACACCACCGAGCCTATGCTG |
| | ATGGAATACCCCGAGGCCATCACCAGACTGGTCACCGGTGCTCAAAGACCACC |
| | TGATCCGGCTCCAGCACCTCTTGGAGCACCTGGACTGCCTAATGGACTGCTGTC |
| | TGGCGACGAGGACTTCAGCTCTATCGCCGACATGGATTTCAGCGCCCTGCTCA |
| | GTGGCGGTGGAAGCGGAGGAAGTGGCAGCGATCTTTCTCACCCTCCACCTAGA |
| | GGCCACCTGGACGAGCTGACAACCACACTGGAATCCATGACCGAGGACCTGA |
| | ACCTGGACAGCCCTCTGACACCCGAGCTGAACGAGATCCTGGACACCTTCCTG |
| | AACGACGAGTGTCTGCTGCACGCCATGCACATCTCTACCGGCCTGAGCATCTTC |
| | GACACCAGCCTGTTTGAGGATGTCGTGTGCTGCCACAGCATCTACGGCAAGAA |
| | GAAGGGCGACATCGACACCTACCGGTACATCGGCAGCTCTGGCACAGGCTGTG |
| | TGGTCATCGTGGGCAGAATCGTGCTGTCTGGCAGCGGAACAAGCGCCCCTATC |
| | ACAGCCTATGCTCAGCAGACAAGAGGCCTGCTGGGCTGCATCATCACAAGCCT |
| | GACCGGCAGAGACAAGAACCAGGTGGAAGGCGAGGTGCAGATCGTGTCTACA |
| | GCTACCCAGACCTTCCTGGCCACCTGTATCAATGGCGTGTGCTGGGCCGTGTAT |
| | CACGGCGCTGGAACCAGAACAATCGCCTCTCCTAAGGGCCCCGTGATCCAGAT |
| | GTACACCAACGTGGACCAGGACCTCGTTGGCTGGCCTGCTCCTCAAGGCAGCA |
| | GAAGCCTGACACCTTGCACCTGTGGCTCCAGCGATCTGTACCTGGTCACCAGA |
| | CACGCCGACGTGATCCCTGTCAGAAGAAGAGGGGATTCCAGAGGCAGCCTGCT |
| | GAGCCCTAGACCTATCAGCTACCTGAAGGGCTCTAGCGGCGGACCTCTGCTTT |
| | GTCCTGCTGGACATGCCGTGGGCCTGTTTAGAGCCGCCGTGTGTACAAGAGGC |
| | GTGGCCAAAGCCGTGGACTTCATCCCCGTGGAAAACCTGGAAACCACCATGCG |
| | GAGCCCCGTGTTCACCGACAATTCTAGCCCTCCAGCCGTGACACTGACACACC |
| | CCATCACCAAGATCGACAGAGAGGTGCTGTACCAAGAGTTCGACGAGATGGA |
| | AGAGTGCAGCCAGCACATGTCTAGACCTGGCGAGAGGCCCTTCCAGTGCCGGA |
| | TCTGCATGCGGAACTTCAGCAACATGAGCAACCTGACCAGACACACCCGGACA |
| | CACACAGGCGAGAAGCCTTTTCAGTGCAGAATCTGTATGCGCAATTTCTCCGA |
| | CAGAAGCGTGCTGCGGAGACACCTGAGAACCCACACCGGCAGCCAGAAACCA |
| | TTCCAGTGTCGCATCTGTATGAGAAACTTTAGCGACCCCTCCAATCTGGCCCGG |
| | CACACCAGAACACATACCGGGGAAAAACCCTTTCAGTGTAGGATATGCATGAG |
| | GAATTTTTCCGACCGGTCCAGCCTGAGGCGGCACCTGAGGACACATACTGGCT |
| | CCCAAAAGCCGTTCCAATGTCGGATATGTATGCGCAACTTTAGCCAGAGCGGC |
| | ACCCTGCACAGACACACAAGAACCCATACTGGCGAGAAACCTTTCCAATGTAG |
| | AATCTGCATGCGAAATTTTTCCCAGCGGCCTAATCTGACCAGGCATCTGAGGA |
| | CCCACCTGAGAGGATCTTaAGTCGACAATCAACCTCtggattacaaaatttgtgaaagattgactg |
| | gtattcttaactatgttgctccttttacgctatgtggatacgctgctttaatgcctttgtatcatgctattgcttcccgtatggctttcattttctc |
| | ctccttgtataaatcctggttgctgtctctttatgaggagttgtggcccgttgtcaggcaacgtggcgtggtgtgcactgtgtttgctgac |
| | gcaacccccactggttggggcattgccaccacctgtcagctcctttccgggactttcgctttccccctccctattgccacggcggaac |
| | tcatcgccgcctgccttgcccgctgctggacaggggctcggctgttgggcactgacaattccgtggtgttgtcggggaaatcatcgt |
| | cctttccttggctgctcgcctgtgttgccacctggattctgcgcgggacgtccttctgctacgtcccttcggccctcaatccagcggac |
| | cttccttcccgcggcctgctgccggctctgcggcctcttccgcgtctacgccttcgccctcagacgagtcggatctccctttgggcc |
| | gcctccccgcgatatcagtggtccaggctctagttttgactcaacaatatcaccagctgaagcctatagagtacgagccatagataa |
| | aataaaagattttatttagtctccagaaaaaggggggaatgaaagaccccacctgtaggtttggcaagctagcaataaaagagccc |
| | acaacccctcactcggggcgccagtcctccgattgactgagtcgcccggccgcttcgagcagacatgataagatacattgatgagt |
| | ttggacaaaccacaactagaatgcagtgaaaaaaatgctttatttgtgaaatttgtgatgctattgctttatttgtaaccattataagctgc |
| | aataaacaagttaacaacaacaattgcattcattttatgtttcaggttcagggggagatgtgggaggttttttaaagcaagtaaaacctc |
| | tacaaatgtggtaaaatcgataaggatcgggtacccgtgtatccaataaaccctcttgcagttgcatccgacttgtggtctcgctgttc |
| | cttgggagggtctcctctgagtgattgactacccgtcagcgggggtctttcacacatgcagcatgtatcaaaattaatttggttttttttct |
| | taagctgtgccttctagttgccagccatctgttgtttgcccctcccccgtgccttccttgaccctggaaggtgccactcccactgtccttt |
| | cctaataaaatgaggaaattgcatcgcattgtctgagtaggtgtcattctattctggggggtggggggggcaggacagcaagggg |
| | gaggattgggaagacaatagcaggcatgctggggatgcggtgggctctatggagatcccgcggtacctcgcgaatgcatctagat |
| | ccaatggcctttttggcccagacatgataagatacattgatgagtttggacaaaccacaactagaatgcagtgaaaaaaatgctttatt |
| | tgtgaaatttgtgatgctattgctttatttgtaaccattataagctgcaataaacaagttgcggccgcttagccctcccacacataaccag |
| | agggcagcaattcacgaatcccaactgccgtcggctgtccatcactgtccttcactatggctttgatcccaggatgcagatcgagaa |
| | gcacctgtcggcaccgtccgcaggggctcaagatgcccctgttctcatttccgatcgcgacgatacaagtcaggttgccagctgcc |
| | gcagcagcagcagtgcccagcaccacgagttctgcacaaggtcccccagtaaaatgatatacattgacaccagtgaagatgcgg |
| | ccgtcgctagagagagctgcgctggcgacgctgtagtcttcagagatggggatgctgttgattgtagccgttgctctttcaatgagg |
| | gtggattcttcttgagacaaaggcttggccatgcggccgccgctcggtgttcgaggccacacgcgtcaccttaatatgcgaagtgg |
| | acctcggaccgcgccgccccgactgcatctgcgtgttcgaattcgccaatgacaagacgctgggcggggtttgtgtcatcatagaa |
| | ctaaagacatgcaaatatatttcttccggggggtaccggcctttttggccATTGGatcggatctggccaaaaaggcccttaagta |
| | tttacattaaatggccatagtacttaaagttacattggcttccttgaaataaacatggagtattcagaatgtgtcataaatatttctaattttatggtt |
| | agatagtatctccattggctttctactttttcttttatttttttttgtcctctgtcttccatttgttgttgttgttgtttgtttgtttgtttgttggt |
| | ggttaatttttttttaaagatcctacactatagttcaagctagactattagctactctgtaacccagggtgaccttgaagtcatgggtagc |
| | ctgctgttttagccttcccacatctaagattacaggtatgagctatcatttttggtatattgattgattgattgattgatgtgtgtgtgtgtgat |
| | tgtgtttgtgtgtgtgaTtgtgTaTatgtgtgtatggTtgtgtgtgaTtgtgtgtatgtatgTTtgtgtgtgaTtgTgtgtgtgtgaT |
| | tgtgcatgtgtgtgtgtgtgaTtgtgtTtatgtgtatgaTtgtgtgtgtgtgtgtgtgtgtgtgtgtgtgtgtgtgtgtgtgtgtgttgtgT |
| | aTaTatatttatggtagtgagagGcaacgctccggctcaggtgtcaggttggtttttgagacagagtctttcacttagcttggaattc |
| | actggccgtcgttttacaacgtcgtgactgggaaaaccctggcgttacccaacttaatcgccttgcagcacatccccctttcgccagc |
| | tggcgtaatagcgaagaggcccgcaccgatcgcccttcccaacagttgcgcagcctgaatggcgaatggcgcctgatgcggtatt |
| | ttctccttacgcatctgtgcggtatttcacaccgcatatggtgcactctcagtacaatctgctctgatgccgcatagttaagccagcccc |
| | gacacccgccaacacccgctgacgcgccctgacgggcttgtctgctcccggcatccgcttacagacaagctgtgaccgtctccgg |
| | gagctgcatgtgtcagaggttttcaccgtcatcaccgaaacgcgcgagacgaaagggcctcgtgatacgcctatttttataggttaat |
| | gtcatgataataatggtttcttagacgtcaggtggcacttttcggggaaatgtgcgcggaacccctatttgtttatttttctaaatacattc |
| | aaatatgtatccgctcatgagacaataaccctgataaatgcttcaataatattgaaaaaggaagagtatgagtattcaacatttccgtgt |
| | cgcccttattcccttttttgcggcattttgccttcctgtttttgctcacccagaaacgctggtgaaagtaaaagatgctgaagatcagttg |
| | ggtgcacgagtgggttacatcgaactggatctcaacagcggtaagatccttgagagttttcgccccgaagaacgttttccaatgatg |
| | agcacttttaaagttctgctatgtggcgcggtattatcccgtattgacgccgggcaagagcaactcggtcgccgcatacactattctc |
| | agaatgacttggttgagtactcaccagtcacagaaaagcatcttacggatggcatgacagtaagagaattatgcagtgctgccataa |
| | ccatgagtgataacactgcggccaacttacttctgacaacgatcggaggaccgaaggagctaaccgcttttttgcacaacatgggg |
| | gatcatgtaactcgccttgatcgttgggaaccggagctgaatgaagccataccaaacgacgagcgtgacaccacgatgcctgtag |
| | caatggcaacaacgttgcgcaaactattaactggcgaactacttactctagcttcccggcaacaattaatagactggatggaggcgg |
| | ataaagttgcaggaccacttctgcgctcggcccttccggctggctggtttattgctgataaatctggagccggtgagcgtgggtctcg |
| | cggtatcattgcagcactggggccagatggtaagccctcccgtatcgtagttatctacacgacggggagtcaggcaactatggatg |
| | aacgaaatagacagatcgctgagataggtgcctcactgattaagcattggtaactgtcagaccaagtttactcatatatactttagattg |
| | atttaaaacttcatttttaatttaaaaggatctaggtgaagatcctttttgataatctcatgaccaaaatcccttaacgtgagttttcgttcca |
| | ctgagcgtcagaccccgtagaaaagatcaaaggatcttcttgagatcctttttttctgcgcgtaatctgctgcttgcaaacaaaaaaac |
| | caccgctaccagcggtggtttgtttgccggatcaagagctaccaactctttttccgaaggtaactggcttcagcagagcgcagatac |
| | caaatactgttcttctagtgtagccgtagttaggccaccacttcaagaactctgtagcaccgcctacatacctcgctctgctaatcctgt |
| | taccagtggctgctgccagtggcgataagtcgtgtcttaccgggttggactcaagacgatagttaccggataaggcgcagcggtcg |
| | ggctgaacggggggttcgtgcacacagcccagcttggagcgaacgacctacaccgaactgagatacctacagcgtgagctatga |
| | gaaagcgccacgcttcccgaagggagaaaggcggacaggtatccggtaagcggcagggtcggaacaggagagcgcacgag |
| | ggagcttccagggggaaacgcctggtatctttatagtcctgtcgggtttcgccacctctgacttgagcgtcgatttttgtgatgctcgtc |
| | aggggggcggagcctatggaaaaacgccagcaacgcggcctttttacggttcctggccttttgctggccttttgctcacatgttctttc |
| | ctgcgttatcccctgattctgtggataaccgtattaccgcctttgagtgagctgataccgctcgccgcagccgaacgaccgagcgca |
| | gcgagtcagtgagcgaggaagcggaagagcgcccaatacgcaaaccgcctctccccgcgcgttggccgattcattaatgcagct |
| | ggcacgacaggtttcccgactggaaagcgggcagtgagcgcaacgcaattaatgtgagttagctcactcattaggcaccccaggc |
| | tttacactttatgcttccggctcgtatgttgtgtggaattgtgagcggataacaatttcacacaggaaacagctatgaccatgattacgc |
| | c (SEQ ID NO: 317) |
| SB05058 | aagcttggaattcgagcttgcatgcctgcaggtcgttacataacttacggtaaatggcccgcctggctgaccgcccaacgaccccc |
| | gcccattgacgtcaataatgacgtatgttcccatagtaacgccaatagggactttccattgacgtcaatgggtggagtatttacggtaa |
| | actgcccacttggcagtacatcaagtgtatcatatgccaagtacgccccctattgacgtcaatgacggtaaatggcccgcctggcatt |
| | atgcccagtacatgaccttatgggactttcctacttggcagtacatctacgtattagtcatcgctattaccatggtgatgcggttttggca |
| | gtacatcaatgggcgtggatagcggtttgactcacggggatttccaagtctccaccccattgacgtcaatgggagtttgttttggcac |
| | caaaatcaacgggactttccaaaatgtgtaacaactccgccccattgacgcaaatgggcggtaggcgtgtacggtgggaggtct |
| | atataagcagagctcaataaaagagcccacaacccctcactcggcgcgccagtcctccgattgactgagtcgcccgggtacccgt |
| | gtatccaataaaccctcttgcagttgcatccgacttgtggtctcgctgttccttgggagggtctcctctgagtgattgactacccgtcag |
| | cgggggtctttcatttgggggctcgtccgagatcgggagacccctgcccagggaccaccgacccaccaccgggaggtaagctg |
| | gccagcaacttatctgtgtctgtccgattgtctagtgtctatgactgattttatgcgcctgcgtcggtactagttagctaactagctctgta |
| | tctggcggacccgtggtggaactgacgagttcggaacacccggccgcaaccctgggagacgtcccagggacttcgggggccgt |
| | ttttgtggcccgacctgagtcctaaaatcccgatcgtttaggactctttggtgcaccccccttagaggagggatatgtggttctggtag |
| | gagacgagaacctaaaacagttcccgcctccgtctgaatttttgctttcggtttgggaccgaagccgcgccgcgcgtcttgtctgctg |
| | cagcatcgttctgtgttgtctctgtctgactgtgtttctgtatttgtctgaaaatatgggccccccctcgagtccccagcatgcctgctatt |
| | ctcttcccaatcctcccccttgctgtcctgccccaccccaccccccagaatagaatgacacctactcagacaatgcgatgcaatttcc |
| | tcattttattaggaaaggacagtgggagtggcaccttccagggtcaaggaaggcacgggggaggggcaaacaacagatggctg |
| | gcaactagaaggcacagttacttaCACAGGCCGCACAGATTCTCTCCGCAGCCGTTCGTTTCT |
| | TCTCCGCTCTCTGCACCGAGGGGCGAAGCAGTAGGTCAGGCAACAGATCACGA |
| | AGATGCCGTTCACGGAGATCAGTGTGATGGCCCAGCTTGGCAGCAGTTGAAGA |
| | GATCCACCGCCACTGCCACCGCCGCCACTACCGCCACCAAAGAAGCTGGAGAT |
| | TGTAGACACGGCGAGTCCCTGTGTAATTCCAGATCCTCCGCCTCCGCTACCACC |
| | TCCGCCGCTAGAGGCGTTCAGGTAGCTCATCACTCTGTCGATGGTCACGGCTCT |
| | GATCCGGAAGGCGTGCAGCAGGATGCACAGCTTGATCTTGGTCTTGTAGAAGT |
| | CGGGTTCTTCCAGGCTAGACTTCTGGGGCACTGTCTCGCTGTTGAAGTTCAGGG |
| | CCTGCATCAGCTCGTCGATCACGGCCAGCATATTCTGGTCCAGGAAGATCTGC |
| | CGCTTGGGGTCCATCAGCAGCTTGGCGTTCATGGTCTTGAATTCCACCTGGTAC |
| | ATCTTCAGGTCCTCGTAGATGCTGCTCAGGCACAGGGCCATCATGAAGGAGGT |
| | CTTTCTGCTGGCCAGGCAAGAGCCGTTGGTGATGAAGCTGGTTTCCCGGCTGTT |
| | CAGGCAGCTCTCGTTCTTGGTCAGTTCCAGAGGCAGGCAGGCTTCCACGGTGC |
| | TGGTCTTATCCTTGGTGATGTCCTCGTGGTCGATTTCCTCGCTGGTGCAGGGGT |
| | AGAATTCCAGGGTCTGTCTGGCCTTCTGCAGCATGTTGGACACGGCTCTCAGCA |
| | GGTTCTGGCTGTGGTGCAGACAAGGGAACATGCCAGGATCAGGAGTGGCCAC |
| | AGGCAGGTTTCTAGATCCGCCGCCAGATCCACCACCTGATCCGCCACCGCTTCC |
| | TCCGCCAGAACATGGCACGCTGGCCCATTCGCTCCAAGAGCTGCTGTAGTACC |
| | GGTCCTGGGCTCTGACGCTGATGCTGGCGTTCTTTCTGCAGATCACGGTGGCGC |
| | TGGTCTTGTCGGTGAACACCCGGTCCTTTTTCTCGCGCTTGGACTTGCCCTGCA |
| | CTTGCACGCAAAAGGTCAGGCTGAAGTAGCTGTGGGGTGTAGACCAGGTGTCG |
| | GGGTACTCCCAGGACACTTCCACCTGTCTGCTGTTCTTCAGAGGCTTCAGCTGC |
| | AGGTTCTTTGGAGGATCGGGCTTGATGATGTCCCGGATGAAAAAGCTGGAGGT |
| | GTAGTTCTCGTACTTCAGCTTGTGCACGGCGTCCACCATCACTTCGATAGGCAG |
| | AGACTCTTCGGCGGCTGGACAGGCGCTGTCCTCTTGGCATTCCACGCTGTACTC |
| | GTATTCTTTGTTGTCGCCCCGCACTCTTTCGGCAGACAGTGTAGCGGCGCCACA |
| | TGTAACGCCCTGAGGATCACTGCTGCCTCTGCTGGACTTCACGCTGAAGGTCA |
| | GGTCGGTGCTGATGGTGGTCAGCCACCAACATGTGAACCGGCCGCTGTAGTTC |
| | TTGGCCTCGCATCTCAGGAAGGTCTTGTTCTTGGGCTCTTTCTGGTCCTTCAGG |
| | ATGTCGGTGCTCCAAATGCCATCCTCTTTCTTGTGGAGCAGCAGCAGGCTGTGG |
| | CTCAGCACTTCTCCGCCTTTGTGACAGGTGTACTGGCCGGCGTCGCCAAACTCT |
| | TTCACTTGGATGGTCAGGGTCTTGCCGCTGCCGAGCACCTCGCTAGACTGATCC |
| | AGTGTCCAGGTGATGCCGTCCTCTTCAGGGGTATCGCAGGTCAGCACCACCAT |
| | CTCGCCAGGAGCATCGGGATACCAGTCCAGTTCCACCACGTACACGTCTTTCTT |
| | CAGCTCCCAGATGGCCACCAGAGGAGAGGCCAGGAACACCAGGCTGAACCAG |
| | CTGATGACCAGCTGCTGGTGACACATCATGGTGGCGACACCGGTACGCGTTGG |
| | CCCCCATTATATACCCTCTAGAACTAGTtatccactccgtgtaagggagagtgagcctcttacgaatcT |
| | TCGGCGTCGACTGCTTCattccgaggcgactgataccTTCGGCGTCGACTGCTTCatacgaaggc |
| | agtccgattcTTCGGCGTCGACTGCTTCaacctttactgagacgggacTTCGGCGTCGACTGCTTC |
| | aaaggcgttgcgaatcctcatgcgattgttacgaaacccgTTAATTAAAGAGCGAGATTCCGTCTCAAA |
| | GAAAAAAAAAGTAATGAAATGAATAAAATGAGTCCTAGAGCCAGTAAATGTC |
| | GTAAATGTCTCAGCTAGTCAGGTAGTAAAAGGTCTCAACTAGGCAGTGGCAGA |
| | GCAGGATTCAAATTCAGGGCTGTTGTGATGCCTCCGCAGACTCTGAGCGCCAC |
| | CTGGTGGTAATTTGTCTGTGCCTCTTCTGACGTGGAAGAACAGCAACTAACAC |
| | ACTAACACGGCATTTACTATGGGCCAGCCATTGTCCATCTAGATGGccgataaaataa |
| | aagattttatttagtctccagaaaaaggggggaatgaaagaccccacctgtaggtttggcaagctagctgcaGTGTGTCAG |
| | TTAGGGTGTGGAAAGTCCCCAGGCTCCCCAGCAGGCAGAAGTATGCAAAGCAT |
| | GCATCTCAATTAGTCAGCAACCAGGTGTGGAAAGTCCCCAGGCTCCCCAGCAG |
| | GCAGAAGTATGCAAAGCATGCATCTCAATTAGTCAGCAACCATAGTCCCGCCC |
| | CTAACTCCGCCCATCCCGCCCCTAACTCCGCCCAGTTCCGCCCATTCTCCGCCC |
| | CATGGCTGACTAATTTTTTTTATTTATGCAGAGGCCGAGGCCGCCTCTGCCTCT |
| | GAGCTATTCCAGAAGTAGTGAGGAGGCTTTTTTGGAGGCCTAGGCTTTTGCAA |
| | AGGATCCGCCACCATGCCCAAGAAAAAGCGGAAGGTGATGTCTAGACCTGGC |
| | GAGAGGCCCTTCCAGTGCCGGATCTGCATGCGGAACTTCAGCAACATGAGCAA |
| | CCTGACCAGACACACCCGGACACACACAGGCGAGAAGCCTTTTCAGTGCAGAA |
| | TCTGTATGCGCAATTTCTCCGACAGAAGCGTGCTGCGGAGACACCTGAGAACC |
| | CACACCGGCAGCCAGAAACCATTCCAGTGTCGCATCTGTATGAGAAACTTTAG |
| | CGACCCCTCCAATCTGGCCCGGCACACCAGAACACATACCGGGGAAAAACCCT |
| | TTCAGTGTAGGATATGCATGAGGAATTTTTCCGACCGGTCCAGCCTGAGGCGG |
| | CACCTGAGGACACATACTGGCTCCCAAAAGCCGTTCCAATGTCGGATATGTAT |
| | GCGCAACTTTAGCCAGAGCGGCACCCTGCACAGACACACAAGAACCCATACTG |
| | GCGAGAAACCTTTCCAATGTAGAATCTGCATGCGAAATTTTTCCCAGCGGCCT |
| | AATCTGACCAGGCATCTGAGGACCCACCTGAGAGGATCTGAGGATGTCGTGTG |
| | CTGCCACAGCATCTACGGCAAGAAGAAGGGCGACATCGACACCTACCGGTAC |
| | ATCGGCAGCTCTGGCACAGGCTGTGTGGTCATCGTGGGCAGAATCGTGCTGTC |
| | TGGCAGCGGAACAAGCGCCCCTATCACAGCCTATGCTCAGCAGACAAGAGGCC |
| | TGCTGGGCTGCATCATCACAAGCCTGACCGGCAGAGACAAGAACCAGGTGGA |
| | AGGCGAGGTGCAGATCGTGTCTACAGCTACCCAGACCTTCCTGGCCACCTGTA |
| | TCAATGGCGTGTGCTGGGCCGTGTATCACGGCGCTGGAACCAGAACAATCGCC |
| | TCTCCTAAGGGCCCCGTGATCCAGATGTACACCAACGTGGACCAGGACCTCGT |
| | TGGCTGGCCTGCTCCTCAAGGCAGCAGAAGCCTGACACCTTGCACCTGTGGCT |
| | CCAGCGATCTGTACCTGGTCACCAGACACGCCGACGTGATCCCTGTCAGAAGA |
| | AGAGGGGATTCCAGAGGCAGCCTGCTGAGCCCTAGACCTATCAGCTACCTGAA |
| | GGGCTCTAGCGGCGGACCTCTGCTTTGTCCTGCTGGACATGCCGTGGGCCTGTT |
| | TAGAGCCGCCGTGTGTACAAGAGGCGTGGCCAAAGCCGTGGACTTCATCCCCG |
| | TGGAAAACCTGGAAACCACCATGCGGAGCCCCGTGTTCACCGACAATTCTAGC |
| | CCTCCAGCCGTGACACTGACACACCCCATCACCAAGATCGACAGAGAGGTGCT |
| | GTACCAAGAGTTCGACGAGATGGAAGAGTGCAGCCAGCACGACGCCCTGGAC |
| | GACTTCGATCTGGATATGCTGGGCAGCGACGCTCTGGATGATTTTGACCTGGA |
| | CATGCTCGGCTCTGATGCACTCGACGATTTCGACCTCGATATGTTGGGATCTGA |
| | TGCCCTTGATGACTTTGATCTCGACATGTTGATCAATAGCCGGTCCAGCGGCAG |
| | CCCCAAGAAGAAGAGAAAAGTCGGCTCTGGCGGCGGATCTGGCGGTTCTGGAT |
| | CTGTTTTGCCCCAAGCTCCTGCTCCTGCACCAGCTCCAGCTATGGTTTCTGCTCT |
| | GGCTCAGGCTCCAGCTCCTGTGCCTGTTCTTGCTCCTGGACCTCCTCAGGCTGT |
| | TGCTCCACCAGCACCTAAACCTACACAGGCCGGCGAGGGAACACTGTCTGAAG |
| | CTCTGCTGCAGCTCCAGTTCGACGACGAAGATCTGGGAGCCCTGCTGGGCAAT |
| | AGCACAGATCCTGCCGTGTTCACCGATCTGGCCAGCGTGGACAATAGCGAGTT |
| | CCAGCAGCTCCTGAACCAGGGCATTCCTGTGGCTCCTCACACCACCGAGCCTA |
| | TGCTGATGGAATACCCCGAGGCCATCACCAGACTGGTCACCGGTGCTCAAAGA |
| | CCACCTGATCCGGCTCCAGCACCTCTTGGAGCACCTGGACTGCCTAATGGACT |
| | GCTGTCTGGCGACGAGGACTTCAGCTCTATCGCCGACATGGATTTCAGCGCCCT |
| | GCTCAGTGGCGGTGGAAGCGGAGGAAGTGGCAGCGATCTTTCTCACCCTCCAC |
| | CTAGAGGCCACCTGGACGAGCTGACAACCACACTGGAATCCATGACCGAGGA |
| | CCTGAACCTGGACAGCCCTCTGACACCCGAGCTGAACGAGATCCTGGACACCT |
| | TCCTGAACGACGAGTGTCTGCTGCACGCCATGCACATCTCTACCGGCCTGAGC |
| | ATCTTCGACACCAGCCTGTTTTaAGTCGACAATCAACCTCtggattacaaaatttgtgaaagatt |
| | gactggtattcttaactatgttgctccttttacgctatgtggatacgctgctttaatgcctttgtatcatgctattgcttcccgtatggctttca |
| | ttttctcctccttgtataaatcctggttgctgtctctttatgaggagttgtggcccgttgtcaggcaacgtggcgtggtgtgcactgtgttt |
| | gctgacgcaacccccactggttggggcattgccaccacctgtcagctcctttccgggactttcgctttccccctccctattgccacgg |
| | cggaactcatcgccgcctgccttgcccgctgctggacaggggctcggctgttgggcactgacaattccgtggtgttgtcggggaa |
| | atcatcgtcctttccttggctgctcgcctgtgttgccacctggattctgcgcgggacgtccttctgctacgtcccttcggccctcaatcc |
| | agcggaccttccttcccgcggcctgctgccggctctgcggcctcttccgcgtctacgccttcgccctcagacgagtcggatctccct |
| | ttgggccgcctccccgcgatatcagtggtccaggctctagttttgactcaacaatatcaccagctgaagcctatagagtacgagcca |
| | tagataaaataaaagattttatttagtctccagaaaaaggggggaatgaaagaccccacctgtaggtttggcaagctagcaataaaa |
| | gagcccacaacccctcactcggggcgccagtcctccgattgactgagtcgcccggccgcttcgagcagacatgataagatacatt |
| | gatgagtttggacaaaccacaactagaatgcagtgaaaaaaatgctttatttgtgaaatttgtgatgctattgctttatttgtaaccattat |
| | aagctgcaataaacaagttaacaacaacaattgcattcattttatgtttcaggttcagggggagatgtgggaggttttttaaagcaagta |
| | aaacctctacaaatgtggtaaaatcgataaggatcgggtacccgtgtatccaataaaccctcttgcagttgcatccgacttgtggtctc |
| | gctgttccttgggagggtctcctctgagtgattgactacccgtcagcgggggtctttcacacatgcagcatgtatcaaaattaatttggt |
| | tttttttcttaagctgtgccttctagttgccagccatctgttgtttgcccctcccccgtgccttccttgaccctggaaggtgccactcccac |
| | tgtcctttcctaataaaatgaggaaattgcatcgcattgtctgagtaggtgtcattctattctggggggtggggggggcaggacagc |
| | aagggggaggattgggaagacaatagcaggcatgctggggatgcggtgggctctatggagatcccgcggtacctcgcgaatgc |
| | atctagatccaatggcctttttggcccagacatgataagatacattgatgagtttggacaaaccacaactagaatgcagtgaaaaaaa |
| | tgctttatttgtgaaatttgtgatgctattgctttatttgtaaccattataagctgcaataaacaagttgcggccgcttagccctcccacac |
| | ataaccagagggcagcaattcacgaatcccaactgccgtcggctgtccatcactgtccttcactatggctttgatcccaggatgcag |
| | atcgagaagcacctgtcggcaccgtccgcaggggctcaagatgcccctgttctcatttccgatcgcgacgatacaagtcaggttgc |
| | cagctgccgcagcagcagcagtgcccagcaccacgagttctgcacaaggtcccccagtaaaatgatatacattgacaccagtgaa |
| | gatgcggccgtcgctagagagagctgcgctggcgacgctgtagtcttcagagatggggatgctgttgattgtagccgttgctctttc |
| | aatgagggtggattcttcttgagacaaaggcttggccatgcggccgccgctcggtgttcgaggccacacgcgtcaccttaatatgc |
| | gaagtggacctcggaccgcgccgccccgactgcatctgcgtgttcgaattcgccaatgacaagacgctgggcggggtttgtgtca |
| | tcatagaactaaagacatgcaaatatatttcttccggggggtaccggcctttttggccATTGGatcggatctggccaaaaaggcc |
| | cttaagtatttacattaaatggccatagtacttaaagttacattggcttccttgaaataaacatggagtattcagaatgtgtcataaatattt |
| | ctaattttaagatagtatctccattggctttctactttttcttttatttttttttgtcctctgtcttccatttgttgttgttgttgtttgtttgtttg |
| | tttgttggttggttggttaatttttttttaaagatcctacactatagttcaagctagactattagctactctgtaacccagggtgaccttgaagtcat |
| | gggtagcctgctgttttagccttcccacatctaagattacaggtatgagctatcatttttggtatattgattgattgattgattgatgtgtgt |
| | gtgtgtgattgtgtttgtgtgtgtgaTtgtgTaTatgtgtgtatggTtgtgtgtgaTtgtgtgtatgtatgTTtgtgtgtgaTtgTgt |
| | gtgtgtgaTtgtgcatgtgtgtgtgtgtgaTtgtgtTtatgtgtatgaTtgtgtgtgtgtgtgtgtgtgtgtgtgtgtgtgtgtgtgtgtg |
| | tgtgttgtgTaTaTatatttatggtagtgagagGcaacgctccggctcaggtgtcaggttggtttttgagacagagtctttcacttag |
| | cttggaattcactggccgtcgttttacaacgtcgtgactgggaaaaccctggcgttacccaacttaatcgccttgcagcacatccccc |
| | tttcgccagctggcgtaatagcgaagaggcccgcaccgatcgcccttcccaacagttgcgcagcctgaatggcgaatggcgcctg |
| | atgcggtattttctccttacgcatctgtgcggtatttcacaccgcatatggtgcactctcagtacaatctgctctgatgccgcatagttaa |
| | gccagccccgacacccgccaacacccgctgacgcgccctgacgggcttgtctgctcccggcatccgcttacagacaagctgtga |
| | ccgtctccgggagctgcatgtgtcagaggttttcaccgtcatcaccgaaacgcgcgagacgaaagggcctcgtgatacgcctatttt |
| | tataggttaatgtcatgataataatggtttcttagacgtcaggtggcacttttcggggaaatgtgcgcggaacccctatttgtttatttttct |
| | aaatacattcaaatatgtatccgctcatgagacaataaccctgataaatgcttcaataatattgaaaaaggaagagtatgagtattcaa |
| | catttccgtgtcgcccttattcccttttttgcggcattttgccttcctgtttttgctcacccagaaacgctggtgaaagtaaaagatgctga |
| | agatcagttgggtgcacgagtgggttacatcgaactggatctcaacagcggtaagatccttgagagttttcgccccgaagaacgtttt |
| | ccaatgatgagcacttttaaagttctgctatgtggcgcggtattatcccgtattgacgccgggcaagagcaactcggtcgccgcata |
| | cactattctcagaatgacttggttgagtactcaccagtcacagaaaagcatcttacggatggcatgacagtaagagaattatgcagtg |
| | ctgccataaccatgagtgataacactgcggccaacttacttctgacaacgatcggaggaccgaaggagctaaccgcttttttgcaca |
| | acatgggggatcatgtaactcgccttgatcgttgggaaccggagctgaatgaagccataccaaacgacgagcgtgacaccacgat |
| | gcctgtagcaatggcaacaacgttgcgcaaactattaactggcgaactacttactctagcttcccggcaacaattaatagactggatg |
| | gaggcggataaagttgcaggaccacttctgcgctcggcccttccggctggctggtttattgctgataaatctggagccggtgagcgt |
| | gggtctcgcggtatcattgcagcactggggccagatggtaagccccccgtatcgtagttatctacacgacggggagtcaggcaa |
| | ctatggatgaacgaaatagacagatcgctgagataggtgcctcactgattaagcattggtaactgtcagaccaagtttactcatatata |
| | ctttagattgatttaaaacttcatttttaatttaaaaggatctaggtgaagatcctttttgataatctcatgaccaaaatcccttaacgtgagt |
| | tttcgttccactgagcgtcagaccccgtagaaaagatcaaaggatcttcttgagatcctttttttctgcgcgtaatctgctgcttgcaaac |
| | aaaaaaaccaccgctaccagcggtggtttgtttgccggatcaagagctaccaactctttttccgaaggtaactggcttcagcagagc |
| | gcagataccaaatactgttcttctagtgtagccgtagttaggccaccacttcaagaactctgtagcaccgcctacatacctcgctctgc |
| | taatcctgttaccagtggctgctgccagtggcgataagtcgtgtcttaccgggttggactcaagacgatagttaccggataaggcgc |
| | agcggtcgggctgaacggggggttcgtgcacacagcccagcttggagcgaacgacctacaccgaactgagatacctacagcgt |
| | gagctatgagaaagcgccacgcttcccgaagggagaaaggcggacaggtatccggtaagcggcagggtcggaacaggagag |
| | cgcacgagggagcttccagggggaaacgcctggtatctttatagtcctgtcgggtttcgccacctctgacttgagcgtcgatttttgt |
| | gatgctcgtcaggggggcggagcctatggaaaaacgccagcaacgcggcctttttacggttcctggccttttgctggccttttgctc |
| | acatgttctttcctgcgttatcccctgattctgtggataaccgtattaccgcctttgagtgagctgataccgctcgccgcagccgaacg |
| | accgagcgcagcgagtcagtgagcgaggaagcggaagagcgcccaatacgcaaaccgcctctccccgcgcgttggccgattc |
| | attaatgcagctggcacgacaggtttcccgactggaaagcgggcagtgagcgcaacgcaattaatgtgagttagctcactcattag |
| | gcaccccaggctttacactttatgcttccggctcgtatgttgtgtggaattgtgagcggataacaatttcacacaggaaacagctatga |
| | ccatgattacgcc (SEQ ID NO: 318) |
| SB04599 | acgcgtgtagtcttatgcaatactcttgtagtcttgcaacatggtaacgatgagttagcaacatgccttacaaggagagaaaaagcac |
| | cgtgcatgccgattggtggaagtaaggtggtacgatcgtgccttattaggaaggcaacagacgggtctgacatggattggacgaa |
| | ccactgaattgccgcattgcagagatattgtatttaagtgcctagctcgatacataaacgggtctctctggttagaccagatctgagcc |
| | tgggagctctctggctaactagggaacccactgcttaagcctcaataaagcttgccttgagtgcttcaagtagtgtgtgcccgtctgtt |
| | gtgtgactctggtaactagagatccctcagacccttttagtcagtgtggaaaatctctagcagtggcgcccgaacagggacctgaaa |
| | gcgaaagggaaaccagagctctctcgacgcaggactcggcttgctgaagcgcgcacggcaagaggcgaggggcggcgactg |
| | gtgagtacgccaaaaattttgactagcggaggctagaaggagagagatgggtgcgagagcgtcagtattaagcgggggagaatt |
| | agatcgcgatgggaaaaaattcggttaaggccagggggaaagaaaaaatataaattaaaacatatagtatgggcaagcagggag |
| | ctagaacgattcgcagttaatcctggcctgttagaaacatcagaaggctgtagacaaatactgggacagctacaaccatcccttcag |
| | TTCGGTTCTTTTTGGTCCTTGAGGATGTCAGTGGACCAGATTCCATCCTCTTTCT |
| | TGTGCAGCAGCAGCAGGGAGTGGGACAGCACTTCGCCACCCTTGTGGCAAGTG |
| | TACTGGCCCGCGTCGCCGAACTCCTTGACTTGAATGGTCAGGGTCTTTCCGCTT |
| | CCGAGCACCTCGGAGCTCTGATCCAGGGTCCAGGTTATGCCGTCCTCTTCTGGC |
| | GTATCGCAAGTCAGCACGACCATTTCTCCAGGGGCGTCCGGGTACCAATCCAG |
| | CTCGACCACGTAGACGTCCTTCTTCAGTTCCCAAATGGCGACCAGAGGGGAAG |
| | CGAGGAACACAAGGGAGAACCAGGAGATGACGAGTTGCTGATGGCACATCAT |
| | GGTGGCGACACCGGTACGCGTTGGCCCCCATTATATACCCTCTAGAACTAGTtat |
| | ccactccgtgtaagggagagtgagcctcttacgaatgCGCGACATCGGCTACGCCgttccgaggcgactgatac |
| | gCGCGACATCGGCTACGCCgtacgaaggcagtccgattgCGCGACATCGGCTACGCCgaccttt |
| | actgagacgggagCGCGACATCGGCTACGCCgaaggcgttgcgaatcctcatgcgattgttacgaaacccgTT |
| | AATTAAAGAGCGAGATTCCGTCTCAAAGAAAAAAAAAGTAATGAAATGAATA |
| | AAATGAGTCCTAGAGCCAGTAAATGTCGTAAATGTCTCAGCTAGTCAGGTAGT |
| | AAAAGGTCTCAACTAGGCAGTGGCAGAGCAGGATTCAAATTCAGGGCTGTTGT |
| | GATGCCTCCGCAGACTCTGAGCGCCACCTGGTGGTAATTTGTCTGTGCCTCTTC |
| | TGACGTGGAAGAACAGCAACTAACACACTAACACGGCATTTACTATGGGCCAG |
| | CCATTGTCCATCTAGATGGccgataaaataaaagattttatttagtctccagaaaaaggggggaatgaaagaccc |
| | cacctgtaggtttggcaagctagctgcagtaacgccattttgcaaggcatggaaaaataccaaaccaagaatagagaagttcagat |
| | caagggcgggtacatgaaaatagctaacgttgggccaaacaggatatctgcggtgagcagtttcggccccggcccggggccaa |
| | gaacagatggtcaccgcagtttcggccccggcccgaggccaagaacagatggtccccagatatggcccaaccctcagcagtttct |
| | taagacccatcagatgtttccaggctcccccaaggacctgaaatgaccctgcgccttatttgaattaaccaatcagcctgcttctcgct |
| | tctgttcgcgcgcttctgcttcccgagctctataaaagagctcacaacccctcactcggcgcgccagtcctccgacagactgagtcg |
| | cccgggGGATCCGCCACCATGCCCAAGAAGAAGCGGAAGGTTTCCCGGCCTGGC |
| | GAGAGGCCTTTCCAGTGCAGAATCTGCATGCGGAACTTCAGCAGACGGCACGG |
| | CCTGGACAGACACACCAGAACACACACAGGCGAGAAACCCTTCCAGTGCCGG |
| | ATCTGTATGAGAAATTTCAGCGACCACAGCAGCCTGAAGCGGCACCTGAGAAC |
| | CCATACCGGCAGCCAGAAACCATTTCAGTGTAGGATATGCATGCGCAATTTCT |
| | CCGTGCGGCACAACCTGACCAGACACCTGAGGACACACACCGGGGAGAAGCC |
| | TTTTCAATGTCGCATATGCATGAGAAACTTCTCTGACCACTCCAACCTGAGCCG |
| | CCACCTCAAAACCCACACCGGCTCTCAAAAGCCCTTCCAATGTAGAATATGTA |
| | TGAGGAACTTTAGCCAGCGGAGCAGCCTCGTGCGCCATCTGAGAACTCACACT |
| | GGCGAAAAGCCGTTTCAATGCCGTATCTGTATGCGCAACTTTAGCGAGAGCGG |
| | CCACCTGAAGAGACATCTGCGCACACACCTGAGAGGCAGCGAGGATGTCGTGT |
| | GCTGCCACAGCATCTACGGAAAGAAGAAGGGCGACATCGACACCTATCGGTA |
| | CATCGGCAGCAGCGGCACAGGCTGTGTTGTGATCGTGGGCAGAATCGTGCTGA |
| | GCGGCTCTGGAACAAGCGCCCCTATCACAGCCTACGCTCAGCAGACAAGAGGC |
| | CTGCTGGGCTGCATCATCACAAGCCTGACCGGCAGAGACAAGAACCAGGTGG |
| | AAGGCGAGGTGCAGATCGTGTCTACAGCTACCCAGACCTTCCTGGCCACCTGT |
| | ATCAATGGCGTGTGCTGGGCCGTGTATCACGGCGCTGGCACAAGAACAATCGC |
| | CTCTCCAAAGGGCCCCGTGATCCAGATGTACACCAACGTGGACCAGGACCTCG |
| | TTGGCTGGCCTGCTCCTCAAGGCAGCAGAAGCCTGACACCTTGCACCTGTGGC |
| | TCCAGCGATCTGTACCTGGTCACCAGACACGCCGACGTGATCCCTGTCAGAAG |
| | AAGAGGGGATTCCAGAGGCAGCCTGCTGAGCCCTAGACCTATCAGCTACCTGA |
| | AGGGCAGCTCTGGCGGACCTCTGCTTTGTCCTGCTGGACATGCCGTGGGCCTGT |
| | TTAGAGCCGCCGTGTGTACAAGAGGCGTGGCCAAAGCCGTGGACTTCATCCCC |
| | GTGGAAAACCTGGAAACCACCATGCGGAGCCCCGTGTTCACCGACAATTCTAG |
| | CCCTCCAGCCGTGACACTGACACACCCCATCACCAAGATCGACAGAGAGGTGC |
| | TGTACCAAGAGTTCGACGAGATGGAAGAGTGCAGCCAGCACGACGCTCTTGAT |
| | GACTTTGACCTGGATATGCTCGGATCAGATGCCCTGGACGATTTCGATCTGGAC |
| | ATGTTGGGGTCTGATGCTCTCGACGACTTCGATCTGGATATGCTTGGAAGTGAC |
| | GCGCTGGATGATTTCGACCTTGACATGCTCATCAATTCTCGATCCAGTGGAAGC |
| | CCGAAAAAGAAACGCAAGGTGGGAAGTGGGGGCGGCTCCGGTGGGAGCGGTA |
| | GTGTATTGCCTCAAGCTCCCGCGCCCGCTCCTGCTCCGGCAATGGTTTCAGCTC |
| | TGGCACAAGCTCCAGCTCCAGTGCCTGTGCTCGCCCCTGGCCCTCCGCAGGCC |
| | GTAGCACCTCCCGCCCCCAAACCGACGCAAGCCGGTGAGGGGACTCTCTCTGA |
| | AGCCTTGCTGCAGCTTCAGTTCGATGATGAAGATCTGGGCGCGCTCTTGGGGA |
| | ACAGCACGGATCCGGCAGTATTTACGGACCTCGCATCAGTTGACAATAGTGAA |
| | TTTCAACAACTTCTTAACCAGGGAATACCGGTTGCGCCCCATACGACGGAACC |
| | TATGCTGATGGAGTACCCTGAAGCTATAACCAGACTCGTAACTGGCGCCCAAC |
| | GCCCGCCCGACCCGGCTCCTGCGCCGCTGGGTGCGCCGGGTCTTCCGAATGGT |
| | CTTCTCTCAGGGGACGAAGATTTCAGTTCCATTGCGGATATGGACTTTTCCGCG |
| | CTCCTGAGTGGGGGTGGCTCTGGAGGCTCTGGTTCCGACCTCAGCCATCCTCCA |
| | CCGAGAGGACACCTCGACGAGCTGACAACCACCCTCGAAAGTATGACGGAAG |
| | ATCTGAACTTGGATTCCCCCCTTACCCCAGAACTGAATGAAATCCTCGATACGT |
| | TCTTGAACGATGAGTGCCTTTTGCACGCCATGCATATATCAACAGGTTTGTCTA |
| | TCTTCGACACGTCCCTCTTTTGAGTCGACAATCAACCTCtggattacaaaatttgtgaaagattg |
| | actggtattcttaactatgttgctccttttacgctatgtggatacgctgctttaatgcctttgtatcatgctattgcttcccgtatggctttcatt |
| | ttctcctccttgtataaatcctggttgctgtctctttatgaggagttgtggcccgttgtcaggcaacgtggcgtggtgtgcactgtgtttg |
| | ctgacgcaacccccactggttggggcattgccaccacctgtcagctcctttccgggactttcgctttccccctccctattgccacggc |
| | ggaactcatcgccgcctgccttgcccgctgctggacaggggctcggctgttgggcactgacaattccgtggtgttgtcggggaaat |
| | catcgtcctttccttggctgctcgcctgtgttgccacctggattctgcgcgggacgtccttctgctacgtcccttcggccctcaatccag |
| | cggaccttccttcccgcggcctgctgccggctctgcggcctcttccgcgtctacgccttcgccctcagacgagtcggatctccctttg |
| | ggccgcctccccgcctggtacctttaagaccaatgacttacaaggcagctgtagatcttagccactttttaaaagaaaaggggggac |
| | tggaagggctaattcactcccaacgaaaataagatctgctttttgcttgtactgggtctctctggttagaccagatctgagcctgggag |
| | ctctctggctaactagggaacccactgcttaagcctcaataaagcttgccttgagtgcttcaagtagtgtgtgcccgtctgttgtgtga |
| | ctctggtaactagagatccctcagacccttttagtcagtgtggaaaatctctagcagtagtagttcatgtcatcttattattcagtatttata |
| | acttgcaaagaaatgaatatcagagagtgagaggaacttgtttattgcagcttataatggttacaaataaagcaatagcatcacaaatt |
| | tcacaaataaagcatttttttcactgcattctagttgtggtttgtccaaactcatcaatgtatcttateatgtctggctctagctatcccgccc |
| | ctaactccgcccagttccgcccattctccgccccatggctgactaattttttttatttatgcagaggccgaggccgcctcggcctctga |
| | gctattccagaagtagtgaggaggcttttttggaggcctagacttttgcagagacggcccaaattcgtaatcatggtcatagctgtttc |
| | ctgtgtgaaattgttatccgctcacaattccacacaacatacgagccggaagcataaagtgtaaagcctggggtgcctaatgagtga |
| | gctaactcacattaattgcgttgcgctcactgcccgctttccagtcgggaaacctgtcgtgccagctgcattaatgaatcggccaacg |
| | cgcggggagaggcggtttgcgtattgggcgctcttccgcttcctcgctcactgactcgctgcgctcggtcgttcggctgcggcgag |
| | cggtatcagctcactcaaaggcggtaatacggttatccacagaatcaggggataacgcaggaaagaacatgtgagcaaaaggcc |
| | agcaaaaggccaggaaccgtaaaaaggccgcgttgctggcgtttttccataggctccgcccccctgacgagcatcacaaaaatcg |
| | acgctcaagtcagaggtggcgaaacccgacaggactataaagataccaggcgtttccccctggaagctccctcgtgcgctctcct |
| | gttccgaccctgccgcttaccggatacctgtccgcctttctcccttcgggaagcgtggcgctttctcatagctcacgctgtaggtatct |
| | cagttcggtgtaggtcgttcgctccaagctgggctgtgtgcacgaaccccccgttcagcccgaccgctgcgccttatccggtaacta |
| | tcgtcttgagtccaacccggtaagacacgacttatcgccactggcagcagccactggtaacaggattagcagagcgaggtatgta |
| | ggcggtgctacagagttcttgaagtggtggcctaactacggctacactagaaggacagtatttggtatctgcgctctgctgaagcca |
| | gttaccttcggaaaaagagttggtagctcttgatccggcaaacaaaccaccgctggtagcggtggtttttttgtttgcaagcagcaga |
| | ttacgcgcagaaaaaaaggatctcaagaagatcctttgatcttttctacggggtctgacgctcagtggaacgaaaactcacgttaag |
| | ggattttggtcatgagattatcaaaaaggatcttcacctagatccttttaaattaaaaatgaagttttaaatcaatctaaagtatatatgagt |
| | aaacttggtctgacagttaccaatgcttaatcagtgaggcacctatctcagcgatctgtctatttcgttcatccatagttgcctgactccc |
| | cgtcgtgtagataactacgatacgggagggcttaccatctggccccagtgctgcaatgataccgcgagacccacgctcaccggct |
| | ccagatttatcagcaataaaccagccagccggaagggccgagcgcagaagtggtcctgcaactttatccgcctccatccagtctat |
| | taattgttgccgggaagctagagtaagtagttcgccagttaatagtttgcgcaacgttgttgccattgctacaggcatcgtggtgtcac |
| | gctcgtcgtttggtatggcttcattcagctccggttcccaacgatcaaggcgagttacatgatcccccatgttgtgcaaaaaagcggtt |
| | agctccttcggtcctccgatcgttgtcagaagtaagttggccgcagtgttatcactcatggttatggcagcactgcataattctcttact |
| | gtcatgccatccgtaagatgcttttctgtgactggtgagtactcaaccaagtcattctgagaatagtgtatgcggcgaccgagttgctc |
| | ttgcccggcgtcaatacgggataataccgcgccacatagcagaactttaaaagtgctcatcattggaaaacgttcttcggggcgaaa |
| | actctcaaggatcttaccgctgttgagatccagttcgatgtaacccactcgtgcacccaactgatcttcagcatcttttactttcaccag |
| | cgtttctgggtgagcaaaaacaggaaggcaaaatgccgcaaaaaagggaataagggcgacacggaaatgttgaatactcatact |
| | cttcctttttcaatattattgaagcatttatcagggttattgtctcatgagcggatacatatttgaatgtatttagaaaaataaacaaatagg |
| | ggttccgcgcacatttccccgaaaagtgccacctgacgtctaagaaaccattattatcatgacattaacctataaaaataggcgtatca |
| | cgaggccctttcgtctcgcgcgtttcggtgatgacggtgaaaacctctgacacatgcagctcccggagacggtcacagcttgtctgt |
| | aagcggatgccgggagcagacaagcccgtcagggcgcgtcagcgggtgttggcgggtgtcggggctggcttaactatgcggc |
| | atcagagcagattgtactgagagtgcaccatatgcggtgtgaaataccgcacagatgcgtaaggagaaaataccgcatcaggcgc |
| | cattcgccattcaggctgcgcaactgttgggaagggcgatcggtgcgggcctcttcgctattacgccagctggcgaaagggggat |
| | gtgctgcaaggcgattaagttgggtaacgccagggttttcccagtcacgacgttgtaaaacgacggccagtgccaagctg (SEQ |
| | ID NO: 319) |

### Example 5: Screening of GPC3 CAR/IL15 Expression Constructs

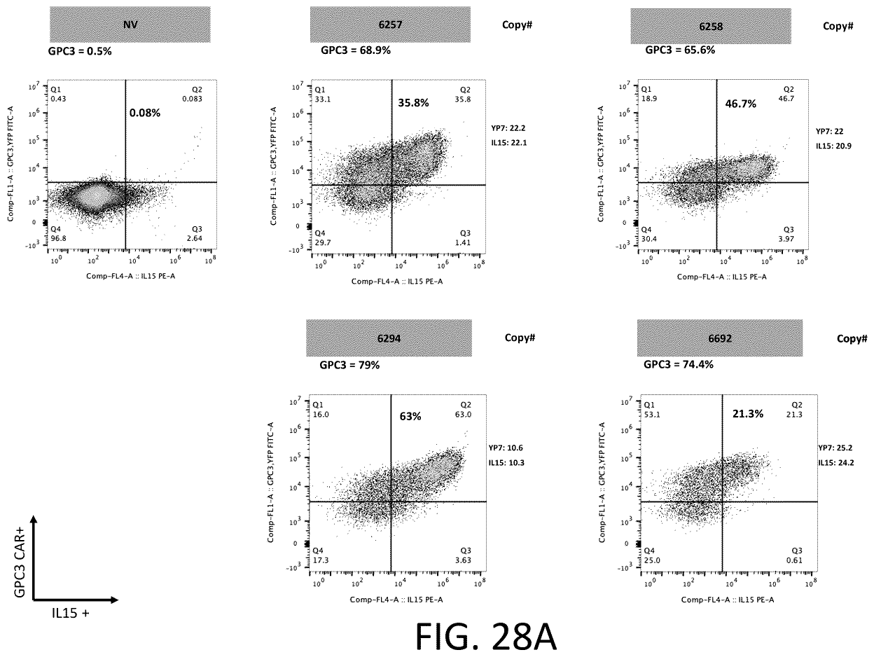

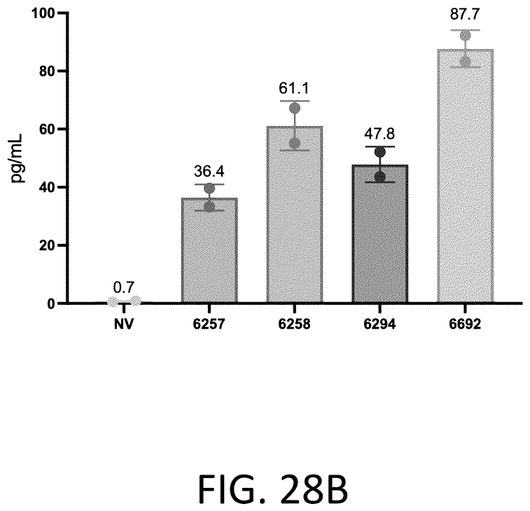

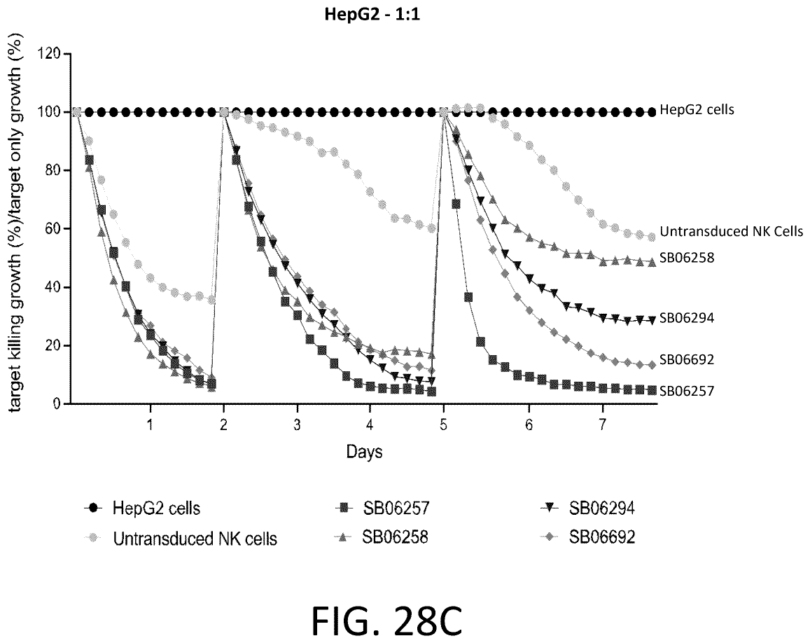

Assessment of the expression and function of the GPC3 CAR/IL15 expression constructs in NK cells was performed. 2e6 NK cells were plated into a 6-well non-TC treated, retronectin coated plate. A single viral transduction via spinoculation (MOI=15) was performed on plated NK cells. The NK cells were transduced using lentivirus or retrovirus containing the expression construct. Expression of the CAR and membrane IL15 were assessed as seen in FIG. 28A. NK cells transduced with constructs SB06257, SB06258, SB06294, and SB06692 exhibited expression of greater than 65% of cells in the gated population. In addition, FIG. 28A shows the measured copy numbers of YP7 and IL15 of each transduced NK cell population.

In addition to CAR expression being assessed, secreted IL15 was also measured using the same expression constructs. To measure the levels of secreted IL15, 200,000 transduced NK cells were suspended in 200 μL of MACS media in the presence of IL2. Secreted IL15 was measured 48 hours after transduction. The concentrations of secreted IL15 were measured for each construct and the results are shown in FIG. 28B.

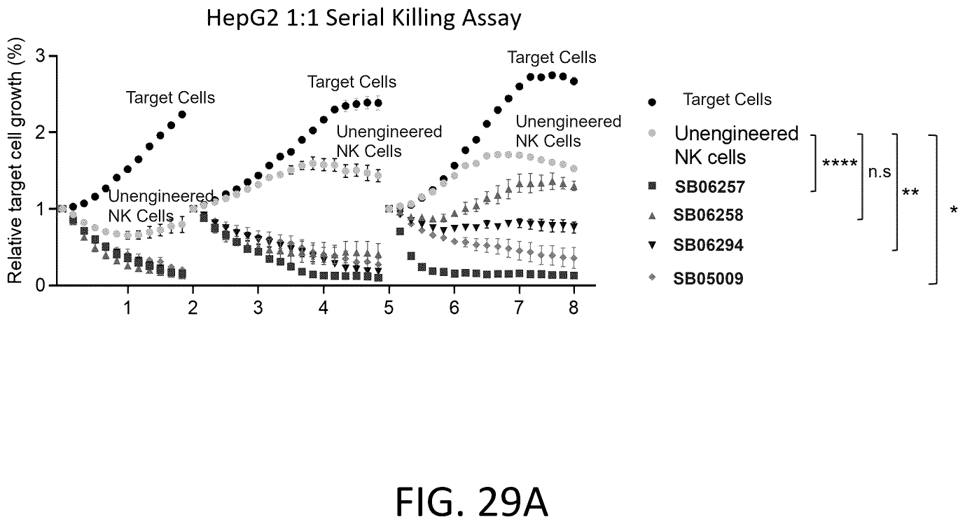

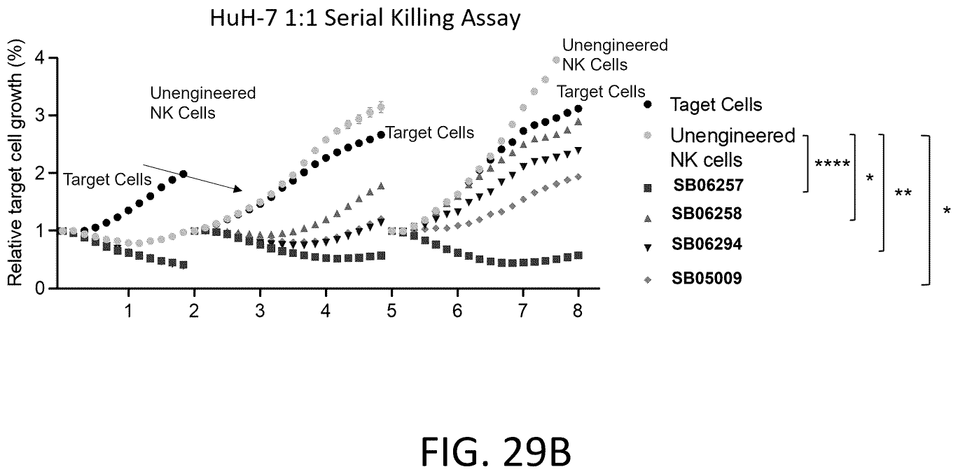

Serial killing by NK cells transduced with the constructs was also assessed. Target cells were added at Days 0, 2, and 5, and target cell killing was measured over the course of the study. Results for serial NK cell killing of HepG2 target cells are shown in FIG. 28C and FIG. 29A. FIG. 29B shows results of serial NK cell killing of HuH-7 target cells.

Table 14 shows the exemplary constructs and their components used in this study.

TABLE 14

| Construct | Base Vector | Co-Stim | Orientation |
|---|---|---|---|
| SB06257 | SinVec | OX40 | CAR 2A crIL15 (T10) |
| SB06258 | SinVec | CD28 | CAR 2A crIL15 (T10) |
| SB06294 | RetroVec | OX40 | crIL15 2A CAR (T10) |
| SB06692 | SinVec | OX40 | crIL15 2A CAR (T-OPT) |

### Example 6: Measuring GPC3 CAR/IL15 Expression and Function in Expanded NK Cells

In this study, the expression and function of GPC3 CAR/IL15 were measured for NK cells that were expanded using the G-Rex (Gas rapid expansion) system.

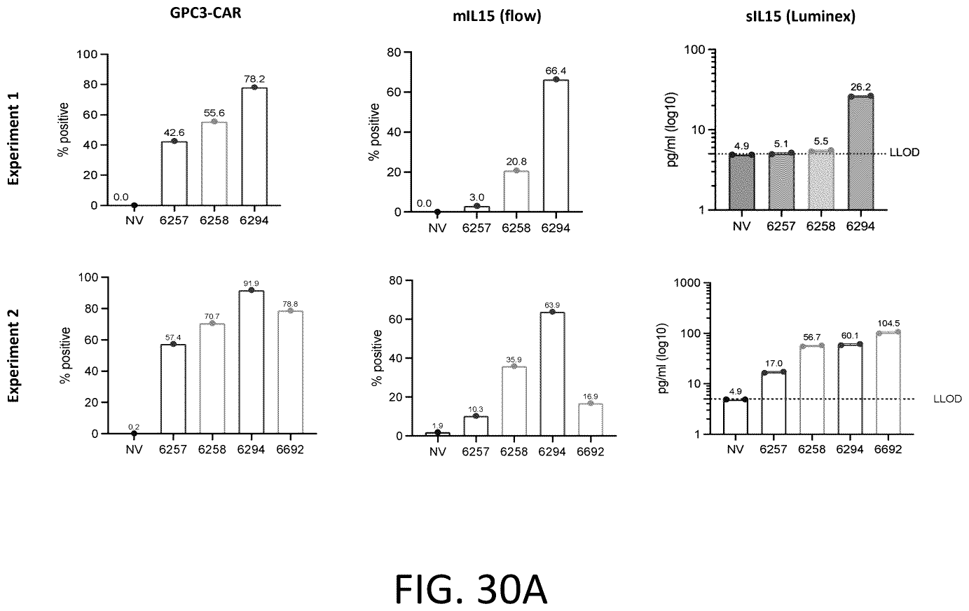

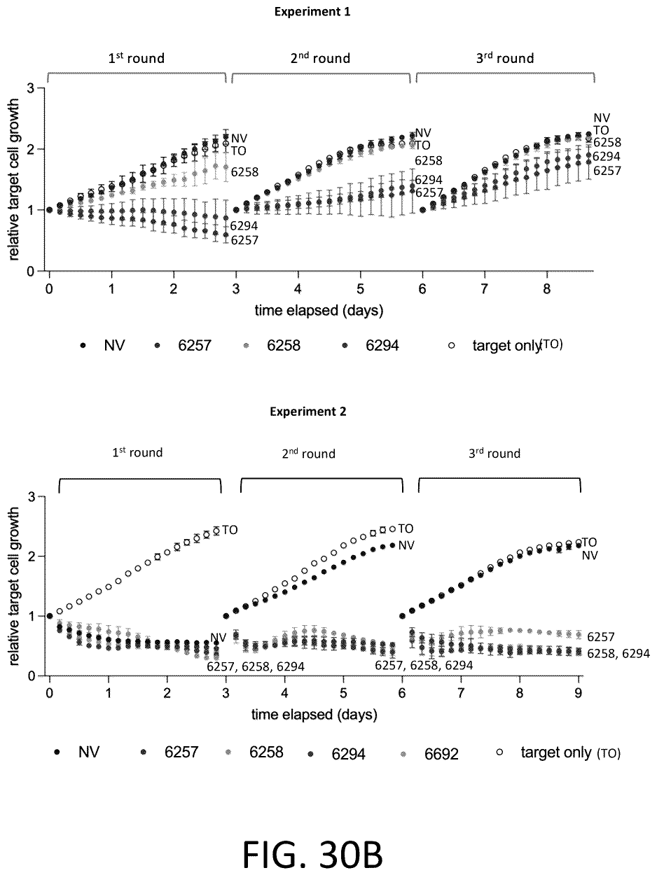

7-day-old donor-derived 7B NK cells (mbIL21/IL15K562 feeders) were transduced and expanded in two different G-Rex experimental methods. Experiment 1 transduced 7-day donor 7B NK cells (mbIL21/IL15K562 feeders) in G-Rex 6M culture containers for 11 days and harvested 11 days after transduction. Experiment 2 transduced 7-day donor 7B NK cells (mbIL21/IL15 K562 feeders) in G-Rex 1L culture containers for 7 days and harvested 10 days after transduction. FIG. 30A demonstrated the effects of the different expansion conditions have on the expression of different proteins of interest in the engineered NK cells. FIG. 30B shows the serial killing assay measurements from the NK Cells derived from the different experiments.

Table 15 shows a summary of the study performed in Example 6. The top number corresponds to results obtained from NK cells expanded using the method of Experiment 1. The bottom number corresponds to results obtained from NK cells expended using the method of Experiment 2.

TABLE 15

| | | | | | % | | | 1^{st} round - | 2^{nd} round- | 3^{rd} round- | CAN | |
|---|---|---|---|---|---|---|---|---|---|---|---|---|
| | Back | Co- | | | GPC3 | % | pg/ml | % HepG2 | % HepG2 | % HepG2 | (copies | |
| SB# | bone | stim | IL15 | Orientation | CAR | mIL15 | sIL15 | killing | killing | killing | per cell) | MOI |
| NV | — | — | — | — | 1.02 | 1.37 | 4.9 | 0 | 0 | 0 | — | — |
| | | | | | 0.2 | 1.9 | 4.9 | 77.2 | 11.0 | 3.9 | | |
| 6257 | SinVec | OX40 | Tace10 | CAR/ | 37.5 | 1.69 | 5.1 | 71.6 | 37.2 | 17.8 | 23.3 | 30.6 |
| | | | | crIL15 | 57.4 | 10.3 | 17.0 | 81.2 | 78.8 | 83.2 | 23.9 | 30.6 |
| 6258 | SinVec | CD28 | Tace10 | CAR/ | 36.8 | 10.7 | 5.5 | 18.3 | 1.4 | 0 | 39.2 | 15.5 |
| | | | | crIL15 | 70.7 | 35.9 | 56.7 | 87.6 | 79.0 | 73.0 | 54.1 | 15.5 |
| 6294 | RetroVec | OX40 | Tace10 | crIL15/ | 78.4 | 58.9 | 26.2 | 58.5 | 33.2 | 12.5 | 41.7 | 10.5 |
| | | | | CAR | 91.9 | 63.9 | 60.1 | 85.2 | 83.8 | 84.2 | 35.0 | 8.8 |
| 6692 | SinVec | OX40 | TaceOPT | crIL15/ | — | — | — | — | — | — | — | — |
| | | | | CAR | 78.8 | 16.9 | 104.5 | 83.4 | 83.0 | 83.2 | 47.5 | 15.0 |

Example 7: Assessment of GPC3 CAR/IL15 Bicistronic Constructs in a Xenograft Tumor Model

The in vivo function of selected engineered NK cells was assessed using a HepG2 xenotransplantation tumor model. Two studies were conducted: a double NK dose and a triple NK dose.

### Double NK Dose In Vivo Xenograft Tumor Model

The tumor was implanted in NSG mice at day 0. Mice were randomized at day 9. NK cells were injected twice over the course of the study on days 10 and 17. Table 16 summarizes the study set-up.

TABLE 16
Summary of double NK dosing in vivo xenograft tumor model

| Tumor model | Group Name | # NKs per dose | Dose day(s) |
|---|---|---|---|
| IP | PBS | — | 10, 17 |
| HepG2, 6e6 | No virus (NV) | 30e6 | |
| | SB06257 | | |
| | SB06258 | | |
| | SB06294* | | |

*Due to cell # limitation, second dose was ~15e6

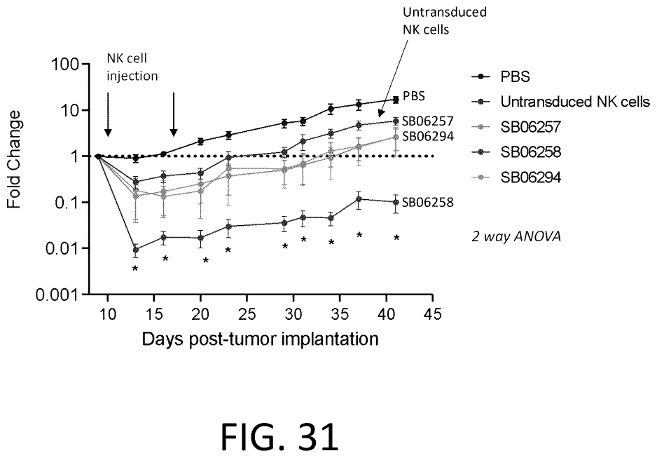

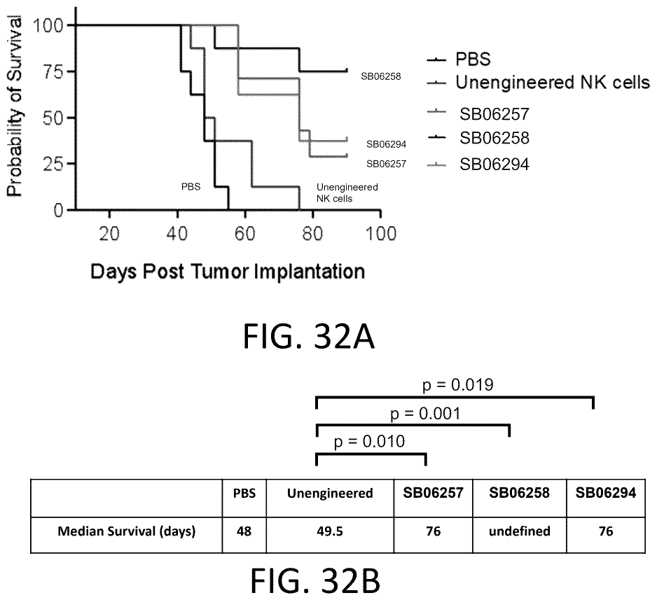

For this survival study, Jackson Labs NSG mice were also injected with 50,000 IU rhIL2 per mouse twice per week. Bioluminescence imaging (BLI), body weight, and overall health measurements were conducted twice a week. Upon euthanizing mice, tumor were collected, weighed, and formalin fixed paraffin embedded (FFPE) for histology. IP fluid and cells were collected from the IP space and the % NK cells were assessed by flow cytometry. FIG. 31 summarizes the results the fold change in normalized mean BLI measurement in the HepG2 xenotransplantation tumor model. SB06258 showed the lowest normalized mean BLI compared to other treatment groups and was found to be statistically significant compared to the no virus (NV) group. FIG. 32A shows a survival curve of animals and FIG. 32B shows a summary of the median survival of each of the treatment groups. Each of the different CAR constructs tested were found to be statistically significant compared to un-engineered NK cells.

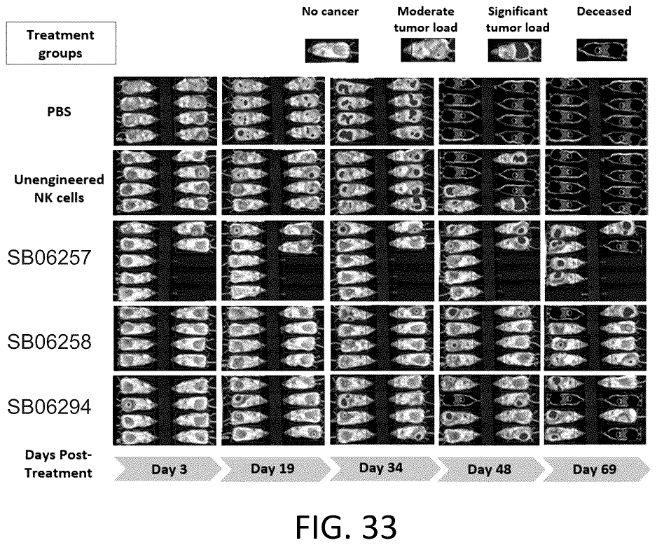

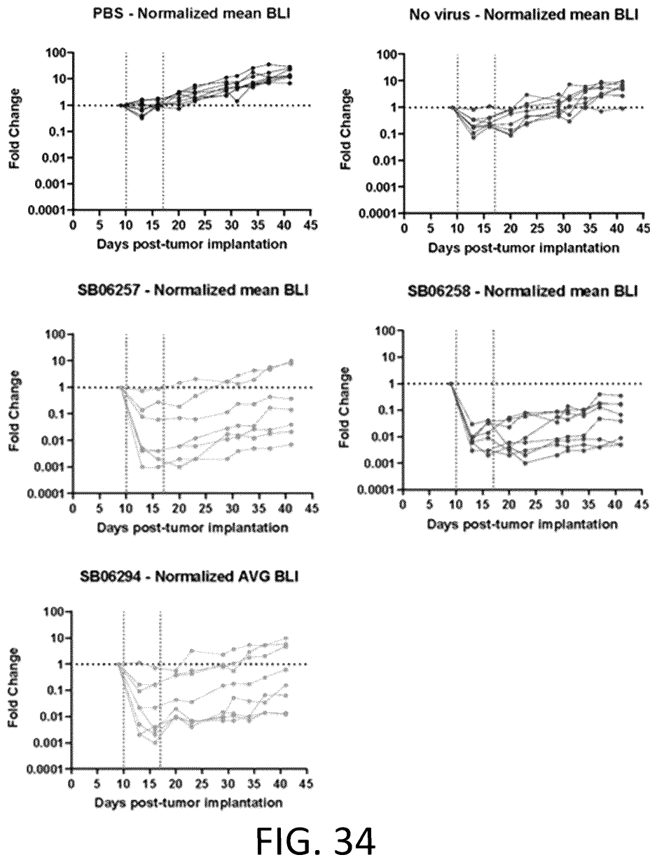

FIG. 33 shows a time course of the mice treated with different CAR-NK cells as measured and observed through bioluminescence imaging (BLI). The animals shown here were imaged 3 days, 10 days, 34 days, 48 days, and 69 days after treatment. In FIG. 34, BLI measurements were normalized to day 10 (first dose).

### Triple Dosing—In Vivo HepG2 Xenograft Tumor Model

The in vivo function of selected engineered NK cells was assessed using a HepG2 xenotransplantation tumor model. The tumor was implanted in NSG mice at day 0 in another in vivo experiments. Mice were randomized at day 9 and day 20. 30e6 NK cells were injected (IP) three times over the course of the study on days 10, 15, and 22. Table 17 summarizes the study set-up. On day 21, half of the mice were euthanized. The other half were euthanized on day 50 of the study. Upon euthanizing mice, tumor were collected, weighed, and formalin fixed paraffin embedded (FFPE) for histology.

TABLE 17
Study Design of HepG2 xenograft model

| Tumor model | Group Name | # NKs per dose | NK dose days |
|---|---|---|---|
| IP | PBS | — | 10, 15, 22 |
| 6e6 | No virus | IP | |
| HepG2 | (NV) | 30e6 | |
| | SB06257 | | |
| | SB06258 | | |
| | SB06294 | | |
| | SB06692 | | |

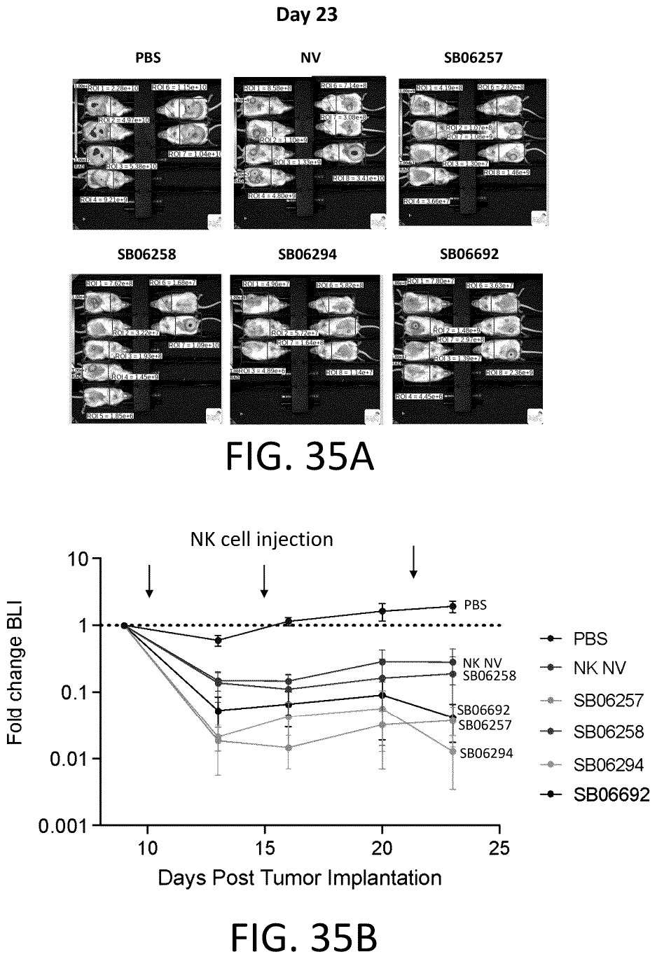

For this survival study, Jackson Labs NSG mice were also injected with 50,000 IU rhIL2 per mouse twice per week. Bioluminescence imaging (BLI), body weight, and overall health measurements were conducted twice a week. IP fluid and cells were collected from the IP space and the % NK cells were assessed by flow cytometry. FIG. 35A shows a representative BLI image at day 23 of the study. FIG. 35B summarizes the results the fold change in normalized mean BLI measurement in the HepG2 xenograft tumor model.

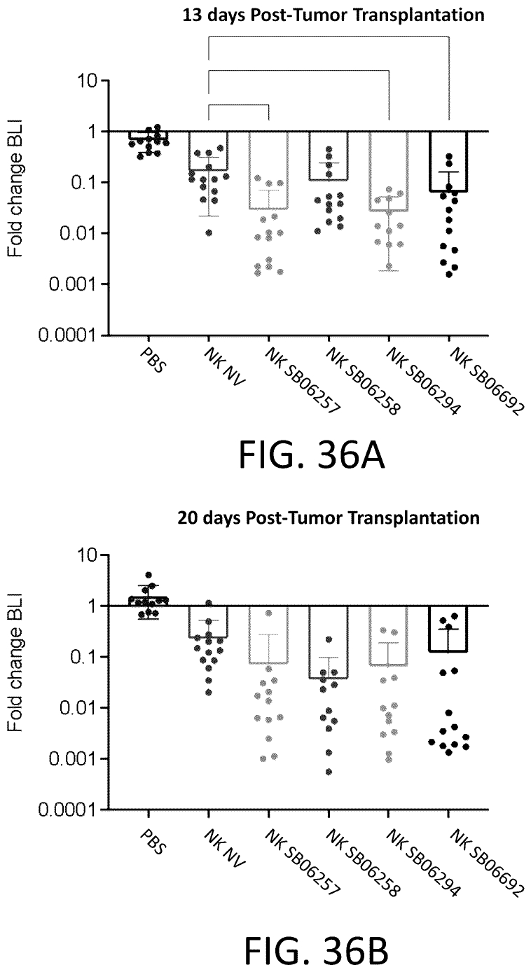

The fold change of BLI measurements were assessed at different stages of the experiments to assess the effect of a single or double dose of the engineered NK cells had an effect. FIG. 36A shows the fold change of BLI measurements on day 13, in which the mice had undergone one dose of the engineered NK cells. FIG. 36B shows the fold change of BLI measurements on day 20, in which the mice had undergone two doses of the engineered NK cells.

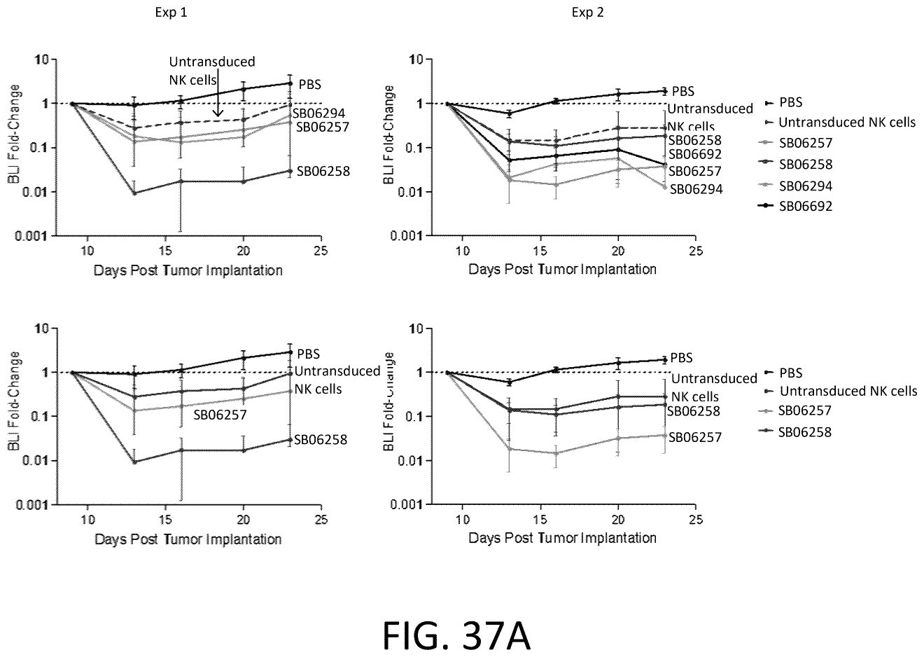

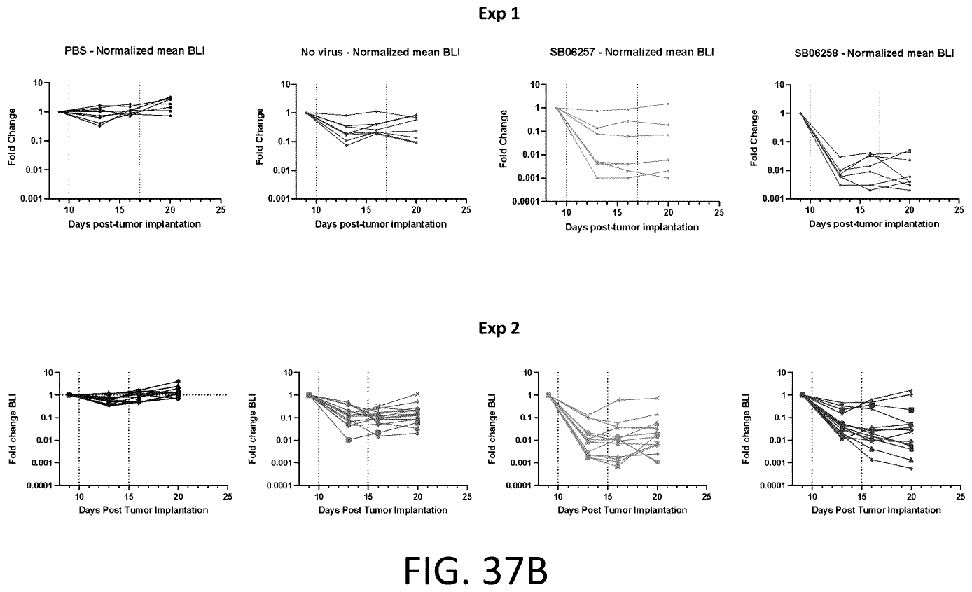

Comparison of the results from the two in vivo experiments are presented in FIG. 37A and FIG. 37B. In FIG. 37A, the different CAR constructs were tested in a xenograft model, plotting fold change of BLI over the course of the study. As seen in FIG. 37A and FIG. 37B, the two in vivo experiments exhibit differences in antitumor function of SB06257 and SB06258. GPC3 CAR- crIL15 NK cell therapy shows statically significant in vivo anti-tumor efficacy compared to unengineered NK cells in an IP HCC (HepG2+luciferase) xenotransplantation model. All 3 groups treated with GPC3 CAR-crIL15 engineered NK cells show significant increased survival over untreated (PBS) and unengineered NK cell-treated groups.

### In Vivo Xenograft Model—Intratumoral Injection of NK Cells

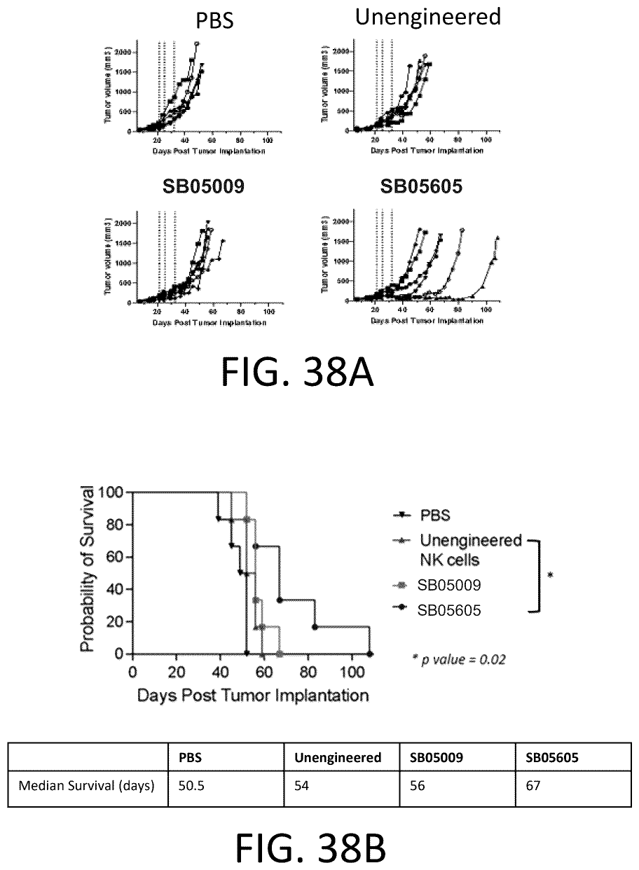

Another experimental approach was used to demonstrate NK-mediated anti-tumor killing for an HepG2 (HCC) subcutaneous xenograft tumor model. In this survival study, mice were injected three times with 3e6 NK cells on days 20, 25, and 32. FIG. 38A demonstrates tumor growth in mice in the absence or presence of injected engineered NK cells. GPC3 CAR-crIL15 NK cell therapy shows significant in vivo anti-tumor efficacy compared to unengineered NK cells injected intratumorally (IT) within a subcutaneous HCC (HepG2+luciferase) xenotransplantation model. NK cells transduced with SB05605 show significantly increased survival over untreated (PBS) and unengineered NK cell-treated groups. Table 18 provides the constructs used for intratumoral injection of NK cells. SB05009 and SB06205 contain IL15 and the GPC3 CAR that are separate, and their expression is driven by separate promoters (SV40 promoter=GPC3 CAR, hPGK promoter=IL15). In addition, these constructs are oriented such that the reading frames are oriented in opposing directions.

TABLE 18

| SEQ ID NO: | Construct | Sequence |
|---|---|---|
| 328 | SB05009 | aagcttgaattcgagcttgcatgcctgcaggtcgttacataacttacggtaaatggcccgcctggctgaccgcccaacgacccccgcc |
| | | cattgacgtcaataatgacgtatgttcccatagtaacgccaatagggactttccattgacgtcaatgggtggagtatttacggtaaactgc |
| | | ccacttggcagtacatcaagtgtatcatatgccaagtacgccccctattgacgtcaatgacggtaaatggcccgcctggcattatgccc |
| | | agtacatgaccttatgggactttcctacttggcagtacatctacgtattagtcatcgctattaccatggtgatgcggttttggcagtacatca |
| | | atgggcgtggatagcggtttgactcacggggatttccaagtctccaccccattgacgtcaatgggagtttgttttggcaccaaaatcaac |
| | | gggactttccaaaatgtcgtaacaactccgccccattgacgcaaatgggcggtaggcgtgtacggtgggaggtctatataagcagag |
| | | ctcaataaaagagcccacaacccctcactcggcgcgccagtcctccgattgactgagtcgcccgggtacccgtgtatccaataaacc |
| | | ctcttgcagttgcatccgacttgtggtctcgctgttccttgggagggtctcctctgagtgattgactacccgtcagcgggggtctttcattt |
| | | gggggctcgtccgagatcgggagacccctgcccagggaccaccgacccaccaccgggaggtaagctggccagcaacttatctgt |
| | | gtctgtccgattgtctagtgtctatgactgattttatgcgcctgcgtcggtactagttagctaactagctctgtatctggcggacccgtggt |
| | | ggaactgacgagttcggaacacccggccgcaaccctgggagacgtcccagggacttcgggggccgtttttgtggcccgacctgagt |
| | | cctaaaatcccgatcgtttaggactctttggtgcaccccccttagaggagggatatgtggttctggtaggagacgagaacctaaaacag |
| | | ttcccgcctccgtctgaatttttgctttcggtttgggaccgaagccgcgccgcgcgtcttgtctgctgcagcatcgttctgtgttgtctctgt |
| | | ctgactgtgtttctgtatttgtctgaaaatatgggccccccctcgagtccccagcatgcctgctattctcttcccaatcctcccccttgctgt |
| | | cctgccccaccccaccccccagaatagaatgacacctactcagacaatgcgatgcaatttcctcattttattaggaaaggacagtggga |
| | | gtggcaccttccagggtcaaggaaggcacgggggaggggcaaacaacagatggctggcaactagaaggcacagcttaAACG |
| | | GGCCGCACAGATTCTCTTCTCAGCCGTTCGTTTCTCCGCCGCTCTCTGCATCTAGG |
| | | GGCGAAGCAGTAGGTCAGGCAGCAGATCACGAAGATGCCGTTCACGGAGATCA |
| | | GTGTGATGGCCCAGCTAGGCAGCAGTTGCAGAGATCCGCCACCACTTCCTCCGC |
| | | CTCCGCTACCGCCTCCGATCAGGCTGAAGATAGGCTCGGGTGTAACTCCGCTTCC |
| | | ACCTCCGCCAGATCCTCCGCCGCCAGAGCTTGTGTTGATGAACATCTGCACGATG |
| | | TGCACGAAGCTCTGCAGGAACTCTTTGATATTCTTCTCTTCCAGTTCCTCGCACTC |
| | | TTTGCAGCCGGACTCGGTCACATTGCCGTTGCTGCTCAGGCTGTTGTTGGCCAGG |
| | | ATGATCAGGTTTTCCACGGTGTCGTGGATGCTGGCGTCGCCGCTTTCCAGGCTGA |
| | | TCACTTGCAGTTCCAGCAGAAAGCACTTCATGGCGGTCACTTTACAGCTAGGGTG |
| | | CACGTCGCTCTCGGTGTACAGTGTGGCGTCGATGTGCATGCTCTGGATCAGGTCC |
| | | TCGATCTTCTTCAGGTCGCTGATCACGTTGACCCAATTGCTGTGCACTCTTGTGG |
| | | CAGCGGCCACCAGAAACAGGATCCAGGTCCAGTCCATGGTGGCGGCacgcgtctgggg |
| | | agagaggtcggtgattcggtcaacgagggagccgactgccgacgtgcgctccggaggcttgcagaatgcggaacaccgcgcggg |
| | | caggaacagggcccacactaccgccccacaccccgcctcccgcaccgccccttcccggccgctgctctcggcgcgccctgctgag |
| | | cagccgctattggccacagcccatcgcggtcggcgcgctgccattgctccctggcgctgtccgtctgcgagggtaatagtgagacgt |
| | | gcggcttccgtttgtcacgtccggcacgccggaaccgcaaggaaccttcccgacttaggggcggagcaggaagcgtcgccggg |
| | | gggcccacaagggtagcggcgaagatccgggtgacgctgcgaacggacgtgaagaatgtgcgagacccagggtcggcgccgct |
| | | gcgtttcccggaaccacgcccagagcagccgcgtccctgcgcaaacccagggctgccttggaaaaggcgcaaccccaaccccga |
| | | attcccgataaaataaaagattttatttagtctccagaaaaaggggggaatgaaagaccccacctgtaggtttggcaagctagctgca |
| | | GTGTGTCAGTTAGGGTGTGGAAAGTCCCCAGGCTCCCCAGCAGGCAGAAGTATG |
| | | CAAAGCATGCATCTCAATTAGTCAGCAACCAGGTGTGGAAAGTCCCCAGGCTCC |
| | | CCAGCAGGCAGAAGTATGCAAAGCATGCATCTCAATTAGTCAGCAACCATAGTC |
| | | CCGCCCCTAACTCCGCCCATCCCGCCCCTAACTCCGCCCAGTTCCGCCCATTCTC |
| | | CGCCCCATGGCTGACTAATTTTTTTTATTTATGCAGAGGCCGAGGCCGCCTCTGC |
| | | CTCTGAGCTATTCCAGAAGTAGTGAGGAGGCTTTTTTGGAGGCCTAGGCTTTTGC |
| | | AAAggatccgccaccATGCTGCTGCTGGTCACATCTCTGCTGCTGTGCGAGCTGCCCCA |
| | | TCCTGCCTTTCTGCTGATCCCTCACATGGAAGTGCAGCTGGTGGAATCTGGCGGA |
| | | GGACTGGTTCAACCTGGCGGCTCTCTGAGACTGTCTTGTGCCGCCAGCGGCTTCA |
| | | CCTTCAACAAGAACGCCATGAACTGGGTCCGACAGGCCCCTGGCAAAGGCCTTG |
| | | AATGGGTCGGACGGATCCGGAACAAGACCAACAACTACGCCACCTACTACGCCG |
| | | ACAGCGTGAAGGCCAGGTTCACCATCTCCAGAGATGACAGCAAGAACAGCCTGT |
| | | ACCTGCAGATGAACTCCCTGAAAACCGAGGACACCGCCGTGTACTATTGCGTGG |
| | | CCGGCAATAGCTTTGCCTACTGGGGACAGGGCACCCTGGTTACAGTTTCTGCTGG |
| | | CGGCGGAGGAAGCGGAGGCGGAGGATCCGGTGGTGGTGGATCTGACATCGTGA |
| | | TGACACAGAGCCCCGATAGCCTGGCCGTGTCTCTGGGAGAAAGAGCCACCATCA |
| | | ACTGCAAGAGCAGCCAGAGCCTGCTGTACTCCAGCAACCAGAAGAACTACCTGG |
| | | CCTGGTATCAGCAAAAGCCCGGCCAGCCTCCTAAGCTGCTGATCTATTGGGCCA |
| | | GCTCCAGAGAAAGCGGCGTGCCCGATAGATTTTCTGGCTCTGGCAGCGGCACCG |
| | | ACTTCACCCTGACAATTTCTAGCCTGCAAGCCGAGGACGTGGCCGTGTATTACTG |
| | | CCAGCAGTACTACAACTACCCTCTGACCTTCGGCCAGGGCACCAAGCTGGAAAT |
| | | CAAATCTGGCGCCCTGAGCAACAGCATCATGTACTTCAGCCACTTCGTGCCCGTG |
| | | TTTCTGCCCGCCAAGCCTACAACAACCCCTGCTCCTAGACCTCCTACACCAGCTC |
| | | CTACAATCGCCAGCCAGCCTCTGTCTCTGAGGCCAGAAGCTTGTAGACCTGCTGC |
| | | AGGCGGAGCCGTGCATACAAGAGGACTGGATTTCGCCTGCGACATCTACATCTG |
| | | GGCCCCTCTGGCTGGAACATGTGGTGTCCTGCTGCTGAGCCTGGTCATCACCCTG |
| | | TACTGCAACCACCGGCGGAGCAAGAGAAGCAGACTGCTGCACAGCGACTACAT |
| | | GAACATGACCCCTAGACGGCCCGGACCTACCAGAAAGCACTACCAGCCTTACGC |
| | | TCCTCCTAGAGACTTCGCCGCCTACCGGTCCAGAGTGAAGTTCAGCAGATCCGCC |
| | | GATGCTCCCGCCTATCAGCAGGGACAGAACCAGCTGTACAACGAGCTGAACCTG |
| | | GGGAGAAGAGAAGAGTACGACGTGCTGGACAAGCGGAGAGGCAGAGATCCTGA |
| | | GATGGGCGGCAAGCCCAGACGGAAGAATCCTCAAGAGGGCCTGTATAATGAGC |
| | | TGCAGAAAGACAAGATGGCCGAGGCCTACAGCGAGATCGGAATGAAGGGCGAG |
| | | CGCAGAAGAGGCAAGGGACACGATGGACTGTACCAGGGCCTGAGCACCGCCAC |
| | | CAAGGATACCTATGATGCCCTGCACATGCAGGCCCTGCCTCCAAGAGGAtaaggatcc |
| | | ggattagtccaatttgttaaagacaggatgggctgcaggaattccgataatcaacctctggattacaaaatttgtgaaagattgactggta |
| | | ttcttaactatgttgctccttttacgctatgtggatacgctgctttaatgcctttgtatcatgctattgcttcccgtatggctttcattttctcctcct |
| | | tgtataaatcctggttgctgtctctttatgaggagttgtggcccgttgtcaggcaacgtggcgtggtgtgcactgtgtttgctgacgcaac |
| | | ccccactggttggggcattgccaccacctgtcagctcctttccgggactttcgctttccccctccctattgccacggcggaactcatcgc |
| | | cgcctgccttgcccgctgctggacaggggctcggctgttgggcactgacaattccgtggtgttgtcggggaagctgacgtcctttccat |
| | | ggctgctcgcctgtgttgccacctggattctgcgcgggacgtccttctgctacgtcccttcggccctcaatccagcggaccttccttccc |
| | | gcggcctgctgccggctctgcggcctcttccgcgtcttcgccttcgccctcagacgagtcggatctccctttgggccgcctccccgcct |
| | | ggagaattcgatatcagtggtccaggctctagttttgactcaacaatatcaccagctgaagcctatagagtacgagccatagataaaata |
| | | aaagattttatttagtctccagaaaaaggggggaatgaaagaccccacctgtaggtttggcaagctagcaataaaagagcccacaacc |
| | | cctcactcggggcgccagtcctccgattgactgagtcgcccggccgcttcgagcagacatgataagatacattgatgagtttggacaa |
| | | accacaactagaatgcagtgaaaaaaatgctttatttgtgaaatttgtgatgctattgctttatttgtaaccattataagctgcaataaacaa |
| | | gttaacaacaacaattgcattcattttatgtttcaggttcagggggagatgtgggaggttttttaaagcaagtaaaacctctacaaatgtgg |
| | | taaaatcgataaggatcgggtacccgtgtatccaataaaccctcttgcagttgcatccgacttgtggtctcgctgttccttgggagggtct |
| | | cctctgagtgattgactacccgtcagcgggggtctttcacacatgcagcatgtatcaaaattaatttggttttttttcttaagctgtgccttct |
| | | agttgccagccatctgttgtttgcccctcccccgtgccttccttgaccctggaaggtgccactcccactgtcctttcctaataaaatgagg |
| | | aaattgcatcgcattgtctgagtaggtgtcattctattctggggggtggggggggcaggacagcaagggggaggattgggaagaca |
| | | atagcaggcatgctggggatgcggtgggctctatggagatcccgcggtacctcgcgaatgcatctagatccaatggcctttttggccc |
| | | agacatgataagatacattgatgagtttggacaaaccacaactagaatgcagtgaaaaaaatgctttatttgtgaaatttgtgatgctattg |
| | | ctttatttgtaaccattataagctgcaataaacaagttgcggccgcttagccctcccacacataaccagagggcagcaattcacgaatcc |
| | | caactgccgtcggctgtccatcactgtccttcactatggctttgatcccaggatgcagatcgagaagcacctgtcggcaccgtccgca |
| | | ggggctcaagatgcccctgttctcatttccgatcgcgacgatacaagtcaggttgccagctgccgcagcagcagcagtgcccagcac |
| | | cacgagttctgcacaaggtcccccagtaaaatgatatacattgacaccagtgaagatgcggccgtcgctagagagagctgcgctggc |
| | | gacgctgtagtcttcagagatggggatgctgttgattgtagccgttgctctttcaatgagggtggattcttcttgagacaaaggcttggcc |
| | | atgcggccgccgctcggtgttcgaggccacacgcgtcaccttaatatgcgaagtggacctcggaccgcgccgccccgactgcatct |
| | | gcgtgttcgaattcgccaatgacaagacgctgggggggtttgtgtcatcatagaactaaagacatgcaaatatatttcttccgggggg |
| | | taccggcctttttggccATTGGatcggatctggccaaaaaggcccttaagtatttacattaaatggccatagtacttaaagttacattg |
| | | gcttccttgaaataaacatggagtattcagaatgtgtcataaatatttctaattttaagatagtatctccattggctttctactttttttttatttttt |
| | | tttgtcctctgtcttccatttgttgttgttgttgtttgtttgtttgtttgttggttggttggttaatttttttttaaagatcctacactatagttcaagcta |
| | | gactattagctactctgtaacccagggtgaccttgaagtcatgggtagcctgctgttttagccttcccacatctaagattacaggtatgag |
| | | ctatcatttttggtatattgattgattgattgattgatgtgtgtgtgtgtgattgtgtttgtgtgtgtgaTtgtgTaTatgtgtgtatggTtgtgt |
| | | gtgaTtgtgtgtatgtatgTTtgtgtgtgaTtgTgtgtgtgtgaTtgtgcatgtgtgtgtgtgtgaTtgtgtTtatgtgtatgaTtgtgt |
| | | gtgtgtgtgtgtgtgtgtgtgtgtgtgtgtgtgtgtgtgtgttgtgTaTaTatatttatggtagtgagagGcaacgctccggctcaggtg |
| | | tcaggttggtttttgagacagagtctttcacttagcttggaattcactggccgtcgttttacaacgtcgtgactgggaaaaccctggcgtta |
| | | cccaacttaatcgccttgcagcacatccccctttcgccagctggcgtaatagcgaagaggcccgcaccgatcgcccttcccaacagtt |
| | | gcgcagcctgaatggcgaatggcgcctgatgcggtattttctccttacgcatctgtgcggtatttcacaccgcatatggtgcactctcag |
| | | tacaatctgctctgatgccgcatagttaagccagccccgacacccgccaacacccgctgacgcgccctgacgggcttgtctgctccc |
| | | ggcatccgcttacagacaagctgtgaccgtctccgggagctgcatgtgtcagaggttttcaccgtcatcaccgaaacgcgcgagacg |
| | | aaagggcctcgtgatacgcctatttttataggttaatgtcatgataataatggtttcttagacgtcaggtggcacttttcggggaaatgtgc |
| | | gcggaacccctatttgtttatttttctaaatacattcaaatatgtatccgctcatgagacaataaccctgataaatgcttcaataatattgaaa |
| | | aaggaagagtatgagtattcaacatttccgtgtcgcccttattcccttttttgcggcattttgccttcctgtttttgctcacccagaaacgctg |
| | | gtgaaagtaaaagatgctgaagatcagttgggtgcacgagtgggttacatcgaactggatctcaacagcggtaagatccttgagagttt |
| | | tcgccccgaagaacgttttccaatgatgagcacttttaaagttctgctatgtggcgcggtattatcccgtattgacgccgggcaagagca |
| | | actcggtcgccgcatacactattctcagaatgacttggttgagtactcaccagtcacagaaaagcatcttacggatggcatgacagtaa |
| | | gagaattatgcagtgctgccataaccatgagtgataacactgcggccaacttacttctgacaacgatcggaggaccgaaggagctaa |
| | | ccgcttttttgcacaacatgggggatcatgtaactcgccttgatcgttgggaaccggagctgaatgaagccataccaaacgacgagcg |
| | | tgacaccacgatgcctgtagcaatggcaacaacgttgcgcaaactattaactggcgaactacttactctagcttcccggcaacaattaat |
| | | agactggatggaggcggataaagttgcaggaccacttctgcgctcggcccttccggctggctggtttattgctgataaatctggagcc |
| | | ggtgagcgtgggtctcgcggtatcattgcagcactggggccagatggtaagccctcccgtatcgtagttatctacacgacggggagt |
| | | caggcaactatggatgaacgaaatagacagatcgctgagataggtgcctcactgattaagcattggtaactgtcagaccaagtttactc |
| | | atatatactttagattgatttaaaacttcatttttaatttaaaaggatctaggtgaagatcctttttgataatctcatgaccaaaatcccttaacgt |
| | | gagttttcgttccactgagcgtcagaccccgtagaaaagatcaaaggatcttcttgagatcctttttttctgcgcgtaatctgctgcttgca |
| | | aacaaaaaaaccaccgctaccagcggtggtttgtttgccggatcaagagctaccaactctttttccgaaggtaactggcttcagcagag |
| | | cgcagataccaaatactgtccttctagtgtagccgtagttaggccaccacttcaagaactctgtagcaccgcctacatacctcgctctgc |
| | | taatcctgttaccagtggctgctgccagtggcgataagtcgtgtcttaccgggttggactcaagacgatagttaccggataaggcgcag |
| | | cggtcgggctgaacggggggttcgtgcacacagcccagcttggagcgaacgacctacaccgaactgagatacctacagcgtgagc |
| | | tatgagaaagcgccacgcttcccgaagggagaaaggcggacaggtatccggtaagcggcagggtcggaacaggagagcgcacg |
| | | agggagcttccagggggaaacgcctggtatctttatagtcctgtcgggtttcgccacctctgacttgagcgtcgatttttgtgatgctcgt |
| | | caggggggcggagcctatggaaaaacgccagcaacgcggcctttttacggttcctggccttttgctggccttttgctcacatgttctttc |
| | | ctgcgttatcccctgattctgtggataaccgtattaccgcctttgagtgagctgataccgctcgccgcagccgaacgaccgagcgcag |
| | | cgagtcagtgagcgaggaagcggaagagcgcccaatacgcaaaccgcctctccccgcggttggccgattcattaatgcagctgg |
| | | cacgacaggtttcccgactggaaagcgggcagtgagcgcaacgcaattaatgtgagttagctcactcattaggcaccccaggctttac |
| | | actttatgcttccggctcgtatgttgtgtggaattgtgagcggataacaatttcacacaggaaacagctatgaccatgattacgcc |
| 329 | SB05605 | aagcttgaattcgagcttgcatgcctgcaggtcgttacataacttacggtaaatggcccgcctggctgaccgcccaacgacccccgcc |
| | | cattgacgtcaataatgacgtatgttcccatagtaacgccaatagggactttccattgacgtcaatgggtggagtatttacggtaaactgc |
| | | ccacttggcagtacatcaagtgtatcatatgccaagtacgccccctattgacgtcaatgacggtaaatggcccgcctggcattatgccc |
| | | agtacatgaccttatgggactttcctacttggcagtacatctacgtattagtcatcgctattaccatggtgatgcggttttggcagtacatca |
| | | atgggcgtggatagcggtttgactcacggggatttccaagtctccaccccattgacgtcaatgggagtttgttttggcaccaaaatcaac |
| | | gggactttccaaaatgtcgtaacaactccgccccattgacgcaaatgggcggtaggcgtgtacggtgggaggtctatataagcagag |
| | | ctcaataaaagagcccacaacccctcactcggcgcgccagtcctccgattgactgagtcgcccgggtacccgtgtatccaataaacc |
| | | ctcttgcagttgcatccgacttgtggtctcgctgttccttgggagggtctcctctgagtgattgactacccgtcagcgggggtctttcattt |
| | | gggggctcgtccgagatcgggagacccctgcccagggaccaccgacccaccaccgggaggtaagctggccagcaacttatctgt |
| | | gtctgtccgattgtctagtgtctatgactgattttatgcgcctgcgtcggtactagttagctaactagctctgtatctggcggacccgtggt |
| | | ggaactgacgagttcggaacacccggccgcaaccctgggagacgtcccagggacttcgggggccgtttttgtggcccgacctgagt |
| | | cctaaaatcccgatcgtttaggactctttggtgcaccccccttagaggagggatatgtggttctggtaggagacgagaacctaaaacag |
| | | ttcccgcctccgtctgaatttttgctttcggtttgggaccgaagccgcgccgcgcgtcttgtctgctgcagcatcgttctgtgttgtctctgt |
| | | ctgactgtgtttctgtatttgtctgaaaatatgggccccccctcgagtccccagcatgcctgctattctcttcccaatcctcccccttgctgt |
| | | cctgccccaccccaccccccagaatagaatgacacctactcagacaatgcgatgcaatttcctcattttattaggaaaggacagtggga |
| | | gtggcaccttccagggtcaaggaaggcacgggggaggggcaaacaacagatggctggcaactagaaggcacagcttaAACG |
| | | GGCCGCACAGATTCTCTTCTCAGCCGTTCGTTTCTCCGCCGCTCTCTGCATCTAGG |
| | | GGCGAAGCAGTAGGTCAGGCAGCAGATCACGAAGATGCCGTTCACGGAGATCA |
| | | GTGTGATGGCCCAGCTAGGCAGCAGTTGCAGAGATCCGCCACCACTTCCTCCGC |
| | | CTCCGCTACCGCCTCCGATCAGGCTGAAGATAGGCTCGGGTGTAACTCCGCTTCC |
| | | ACCTCCGCCAGATCCTCCGCCGCCAGAGCTTGTGTTGATGAACATCTGCACGATG |
| | | TGCACGAAGCTCTGCAGGAACTCTTTGATATTCTTCTCTTCCAGTTCCTCGCACTC |
| | | TTTGCAGCCGGACTCGGTCACATTGCCGTTGCTGCTCAGGCTGTTGTTGGCCAGG |
| | | ATGATCAGGTTTTCCACGGTGTCGTGGATGCTGGCGTCGCCGCTTTCCAGGCTGA |
| | | TCACTTGCAGTTCCAGCAGAAAGCACTTCATGGCGGTCACTTTACAGCTAGGGTG |
| | | CACGTCGCTCTCGGTGTACAGTGTGGCGTCGATGTGCATGCTCTGGATCAGGTCC |
| | | TCGATCTTCTTCAGGTCGCTGATCACGTTGACCCAATTGCTGTGCACTCTTGTGG |
| | | CAGCGGCCACCAGAAACAGGATCCAGGTCCAGTCCATGGTGGCGGCacgcgtctgggg |
| | | agagaggtcggtgattcggtcaacgagggagccgactgccgacgtgcgctccggaggcttgcagaatgcggaacaccgcgcggg |
| | | caggaacagggcccacactaccgccccacaccccgcctcccgcaccgccccttcccggccgctgctctcggcgcgccctgctgag |
| | | cagccgctattggccacagcccatcgcggtcggcgcgctgccattgctccctggcgctgtccgtctgcgagggtaatagtgagacgt |
| | | gcggcttccgtttgtcacgtccggcacgccgcgaaccgcaaggaaccttcccgacttaggggcggagcaggaagcgtcgccggg |
| | | gggcccacaagggtagcggcgaagatccgggtgacgctgcgaacggacgtgaagaatgtgcgagacccagggtcggcgccgct |
| | | gcgtttcccggaaccacgcccagagcagccgcgtccctgcgcaaacccagggctgccttggaaaaggcgcaaccccaaccccga |
| | | attcccgataaaataaaagattttatttagtctccagaaaaaggggggaatgaaagaccccacctgtaggtttggcaagctagctgca |
| | | GTGTGTCAGTTAGGGTGTGGAAAGTCCCCAGGCTCCCCAGCAGGCAGAAGTATG |
| | | CAAAGCATGCATCTCAATTAGTCAGCAACCAGGTGTGGAAAGTCCCCAGGCTCC |
| | | CCAGCAGGCAGAAGTATGCAAAGCATGCATCTCAATTAGTCAGCAACCATAGTC |
| | | CCGCCCCTAACTCCGCCCATCCCGCCCCTAACTCCGCCCAGTTCCGCCCATTCTC |
| | | CGCCCCATGGCTGACTAATTTTTTTTATTTATGCAGAGGCCGAGGCCGCCTCTGC |
| | | CTCTGAGCTATTCCAGAAGTAGTGAGGAGGCTTTTTTGGAGGCCTAGGCTTTTGC |
| | | AAAggatccgccaccATGCTGCTGCTGGTCACATCTCTGCTGCTGTGCGAGCTGCCCCA |
| | | TCCTGCCTTTCTGCTGATCCCTCACATGGAAGTGCAGCTGGTGGAATCTGGCGGA |
| | | GGACTGGTTCAACCTGGCGGCTCTCTGAGACTGTCTTGTGCCGCCAGCGGCTTCA |
| | | CCTTCAACAAGAACGCCATGAACTGGGTCCGACAGGCCCCTGGCAAAGGCCTTG |
| | | AATGGGTCGGACGGATCCGGAACAAGACCAACAACTACGCCACCTACTACGCCG |
| | | ACAGCGTGAAGGCCAGGTTCACCATCTCCAGAGATGACAGCAAGAACAGCCTGT |
| | | ACCTGCAGATGAACTCCCTGAAAACCGAGGACACCGCCGTGTACTATTGCGTGG |
| | | CCGGCAATAGCTTTGCCTACTGGGGACAGGGCACCCTGGTTACAGTTTCTGCTGG |
| | | CGGCGGAGGAAGCGGAGGCGGAGGATCCGGTGGTGGTGGATCTGACATCGTGA |
| | | TGACACAGAGCCCCGATAGCCTGGCCGTGTCTCTGGGAGAAAGAGCCACCATCA |
| | | ACTGCAAGAGCAGCCAGAGCCTGCTGTACTCCAGCAACCAGAAGAACTACCTGG |
| | | CCTGGTATCAGCAAAAGCCCGGCCAGCCTCCTAAGCTGCTGATCTATTGGGCCA |
| | | GCTCCAGAGAAAGCGGCGTGCCCGATAGATTTTCTGGCTCTGGCAGCGGCACCG |
| | | ACTTCACCCTGACAATTTCTAGCCTGCAAGCCGAGGACGTGGCCGTGTATTACTG |
| | | CCAGCAGTACTACAACTACCCTCTGACCTTCGGCCAGGGCACCAAGCTGGAAAT |
| | | CAAGACCACCACACCAGCTCCTCGGCCACCAACTCCAGCTCCAACAATTGCCAG |
| | | CCAGCCTCTGTCTCTGAGGCCCGAAGCTTGTAGACCTGCTGCAGGCGGAGCCGT |
| | | GCATACAAGAGGACTGGATTTCGCCTGCGACATCTACATCTGGGCCCCTCTGGCT |
| | | GGAACATGTGGTGTCTTGCTGCTGAGCCTGGTCATCACCAAGCGGGGCAGAAAG |
| | | AAGCTGCTGTACATCTTCAAGCAGCCCTTCATGCGGCCCGTGCAGACCACACAA |
| | | GAGGAAGATGGCTGCAGCTGTCGGTTCCCCGAGGAAGAAGAAGGCGGCTGCGA |
| | | GCTGAGAGTGAAGTTCAGCAGGAGCGCAGACGCCCCCGCGTACAAGCAGGGCC |
| | | AGAACCAGCTCTATAACGAGCTCAATCTAGGACGAAGAGAGGAGTACGATGTTT |
| | | TGGACAAGAGACGTGGCCGGGACCCTGAGATGGGGGGAAAGCCGAGAAGGAAG |
| | | AACCCTCAGGAAGGCCTGTACAATGAACTGCAGAAAGATAAGATGGCGGAGGC |
| | | CTACAGTGAGATTGGGATGAAAGGCGAGCGCCGGAGGGGCAAGGGGCACGATG |
| | | GCCTTTACCAGGGTCTCAGTACAGCCACCAAGGACACCTACGACGCCCTTCACA |
| | | TGCAGGCCCTGCCCCCTCGCtaaggatccggattagtccaatttgttaaagacaggatgggctgcaggaattccgat |
| | | aatcaacctctggattacaaaatttgtgaaagattgactggtattcttaactatgttgctccttttacgctatgtggatacgctgctttaatgcc |
| | | tttgtatcatgctattgcttcccgtatggctttcattttctcctccttgtataaatcctggttgctgtctctttatgaggagttgtggcccgttgtc |
| | | aggcaacgtggcgtggtgtgcactgtgtttgctgacgcaacccccactggttggggcattgccaccacctgtcagctcctttccggga |
| | | ctttcgctttccccctccctattgccacggcggaactcatcgccgcctgccttgcccgctgctggacaggggctcggctgttgggcact |
| | | gacaattccgtggtgttgtcggggaagctgacgtcctttccatggctgctcgcctgtgttgccacctggattctgcgcgggacgtccttc |
| | | tgctacgtcccttcggccctcaatccagcggaccttccttcccgcggcctgctgccggctctgcggcctcttccgcgtcttcgccttcgc |
| | | cctcagacgagtcggatctccctttgggccgcctccccgcctggagaattcgatatcagtggtccaggctctagttttgactcaacaata |
| | | tcaccagctgaagcctatagagtacgagccatagataaaataaaagattttatttagtctccagaaaaaggggggaatgaaagacccc |
| | | acctgtaggtttggcaagctagcaataaaagagcccacaacccctcactcggggcgccagtcctccgattgactgagtcgcccggcc |
| | | gcttcgagcagacatgataagatacattgatgagtttggacaaaccacaactagaatgcagtgaaaaaaatgctttatttgtgaaatttgt |
| | | gatgctattgctttatttgtaaccattataagctgcaataaacaagttaacaacaacaattgcattcattttatgtttcaggttcagggggaga |
| | | tgtgggaggttttttaaagcaagtaaaacctctacaaatgtggtaaaatcgataaggatcgggtacccgtgtatccaataaaccctcttgc |
| | | agttgcatccgacttgtggtctcgctgttccttgggagggtctcctctgagtgattgactacccgtcagcgggggtctttcacacatgca |
| | | gcatgtatcaaaattaatttggttttttttcttaagctgtgccttctagttgccagccatctgttgtttgcccctcccccgtgccttccttgaccc |
| | | tggaaggtgccactcccactgtcctttcctaataaaatgaggaaattgcatcgcattgtctgagtaggtgtcattctattctggggggtgg |
| | | ggtggggcaggacagcaagggggaggattgggaagacaatagcaggcatgctggggatgcggtgggctctatggagatcccgc |
| | | ggtacctcgcgaatgcatctagatccaatggcctttttggcccagacatgataagatacattgatgagtttggacaaaccacaactagaa |
| | | tgcagtgaaaaaaatgctttatttgtgaaatttgtgatgctattgctttatttgtaaccattataagctgcaataaacaagttgcggccgctta |
| | | gccctcccacacataaccagagggcagcaattcacgaatcccaactgccgtcggctgtccatcactgtccttcactatggctttgatcc |
| | | caggatgcagatcgagaagcacctgtcggcaccgtccgcaggggctcaagatgcccctgttctcatttccgatcgcgacgatacaag |
| | | tcaggttgccagctgccgcagcagcagcagtgcccagcaccacgagttctgcacaaggtcccccagtaaaatgatatacattgacac |
| | | cagtgaagatgcggccgtcgctagagagagctgcgctggcgacgctgtagtcttcagagatggggatgctgttgattgtagccgttg |
| | | ctctttcaatgagggtggattcttcttgagacaaaggcttggccatgcggccgccgctcggtgttcgaggccacacgcgtcaccttaat |
| | | atgcgaagtggacctcggaccgcgccgccccgactgcatctgcgtgttcgaattcgccaatgacaagacgctgggggggtttgtgt |
| | | catcatagaactaaagacatgcaaatatatttcttccggggggtaccggcctttttggccATTGGatcggatctggccaaaaaggc |
| | | ccttaagtatttacattaaatggccatagtacttaaagttacattggcttccttgaaataaacatggagtattcagaatgtgtcataaatatttc |
| | | taattttaagatagtatctccattggctttctactttttcttttatttttttttgtcctctgtcttccatttgttgttgttgttgtttgtttgtttgttt |
| | | gttggttggttggttaatttttttttaaagatcctacactatagttcaagctagactattagctactctgtaacccagggtgaccttgaagtcatgggta |
| | | gcctgctgttttagccttcccacatctaagattacaggtatgagctatcatttttggtatattgattgattgattgattgatgtgtgtgtgtgtga |
| | | ttgtgtttgtgtgtgtgaTtgtgTaTatgtgtgtatggTtgtgtgtgaTtgtgtgtatgtatgTTtgtgtgtgaTtgTgtgtgtgtgaTt |
| | | gtgcatgtgtgtgtgtgtgaTtgtgtTtatgtgtatgaTtgtgtgtgtgtgtgtgtgtgtgtgtgtgtgtgtgtgtgtgtgtgtgttgtgTaT |
| | | a TatatttatggtagtgagagGcaacgctccggctcaggtgtcaggttggtttttgagacagagtctttcacttagcttggaattcactg |
| | | gccgtcgttttacaacgtcgtgactgggaaaaccctggcgttacccaacttaatcgccttgcagcacatccccctttcgccagctggcg |
| | | taatagcgaagaggcccgcaccgatcgcccttcccaacagttgcgcagcctgaatggcgaatggcgcctgatgcggtattttctcctt |
| | | acgcatctgtgcggtatttcacaccgcatatggtgcactctcagtacaatctgctctgatgccgcatagttaagccagccccgacaccc |
| | | gccaacacccgctgacgcgccctgacgggcttgtctgctcccggcatccgcttacagacaagctgtgaccgtctccgggagctgcat |
| | | gtgtcagaggttttcaccgtcatcaccgaaacgcgcgagacgaaagggcctcgtgatacgcctatttttataggttaatgtcatgataat |
| | | aatggtttcttagacgtcaggtggcacttttcggggaaatgtgcgcggaacccctatttgtttatttttctaaatacattcaaatatgtatccg |
| | | ctcatgagacaataaccctgataaatgcttcaataatattgaaaaaggaagagtatgagtattcaacatttccgtgtcgcccttattcccttt |
| | | tttgcggcattttgccttcctgtttttgctcacccagaaacgctggtgaaagtaaaagatgctgaagatcagttgggtgcacgagtgggtt |
| | | acatcgaactggatctcaacagcggtaagatccttgagagttttcgccccgaagaacgttttccaatgatgagcacttttaaagttctgct |
| | | atgtggcgcggtattatcccgtattgacgccgggcaagagcaactcggtcgccgcatacactattctcagaatgacttggttgagtact |
| | | caccagtcacagaaaagcatcttacggatggcatgacagtaagagaattatgcagtgctgccataaccatgagtgataacactgcggc |
| | | caacttacttctgacaacgatcggaggaccgaaggagctaaccgcttttttgcacaacatgggggatcatgtaactcgccttgatcgttg |
| | | ggaaccggagctgaatgaagccataccaaacgacgagcgtgacaccacgatgcctgtagcaatggcaacaacgttgcgcaaactat |
| | | taactggcgaactacttactctagcttcccggcaacaattaatagactggatggaggcggataaagttgcaggaccacttctgcgctcg |
| | | gcccttccggctggctggtttattgctgataaatctggagccggtgagcgtgggtctcgcggtatcattgcagcactggggccagatg |
| | | gtaagccctcccgtatcgtagttatctacacgacggggagtcaggcaactatggatgaacgaaatagacagatcgctgagataggtg |
| | | cctcactgattaagcattggtaactgtcagaccaagtttactcatatatactttagattgatttaaaacttcatttttaatttaaaaggatctagg |
| | | tgaagatcctttttgataatctcatgaccaaaatcccttaacgtgagttttcgttccactgagcgtcagaccccgtagaaaagatcaaagg |
| | | atcttcttgagatcctttttttctgcgcgtaatctgctgcttgcaaacaaaaaaaccaccgctaccagcggtggtttgtttgccggatcaag |
| | | agctaccaactctttttccgaaggtaactggcttcagcagagcgcagataccaaatactgtccttctagtgtagccgtagttaggccacc |
| | | acttcaagaactctgtagcaccgcctacatacctcgctctgctaatcctgttaccagtggctgctgccagtggcgataagtcgtgtcttac |
| | | cgggttggactcaagacgatagttaccggataaggcgcagcggtcgggctgaacggggggttcgtgcacacagcccagcttggag |
| | | cgaacgacctacaccgaactgagatacctacagcgtgagctatgagaaagcgccacgcttcccgaagggagaaaggggacagg |
| | | tatccggtaagcggcagggtcggaacaggagagcgcacgagggagcttccagggggaaacgcctggtatctttatagtcctgtcgg |
| | | gtttcgccacctctgacttgagcgtcgatttttgtgatgctcgtcaggggggggagcctatggaaaaacgccagcaacgcggcctttt |
| | | tacggttcctggccttttgctggccttttgctcacatgttctttcctgcgttatcccctgattctgtggataaccgtattaccgcctttgagtga |
| | | gctgataccgctcgccgcagccgaacgaccgagcgcagcgagtcagtgagcgaggaagcggaagagcgcccaatacgcaaacc |
| | | gcctctccccgcgcgttggccgattcattaatgcagctggcacgacaggtttcccgactggaaagcgggcagtgagcgcaacgcaa |
| | | ttaatgtgagttagctcactcattaggcaccccaggctttacactttatgcttccggctcgtatgttgtgtggaattgtgagcggataacaat |
| | | ttcacacaggaaacagctatgaccatgattacgcc |

### Example 8: Assessment of Grazoprevir Induction of IL12 in Natural Killer Cells

For this study, the induction of IL12 was measured in the presence and absence of grazoprevir, an inhibitor of the HCV NS3 protease. The construct used in this study has been previously described in Example 2. Two regulatable IL12 constructs demonstrated controlled crIL12 expression by GRZ in a dose-response manner and show low donor-to-donor variability

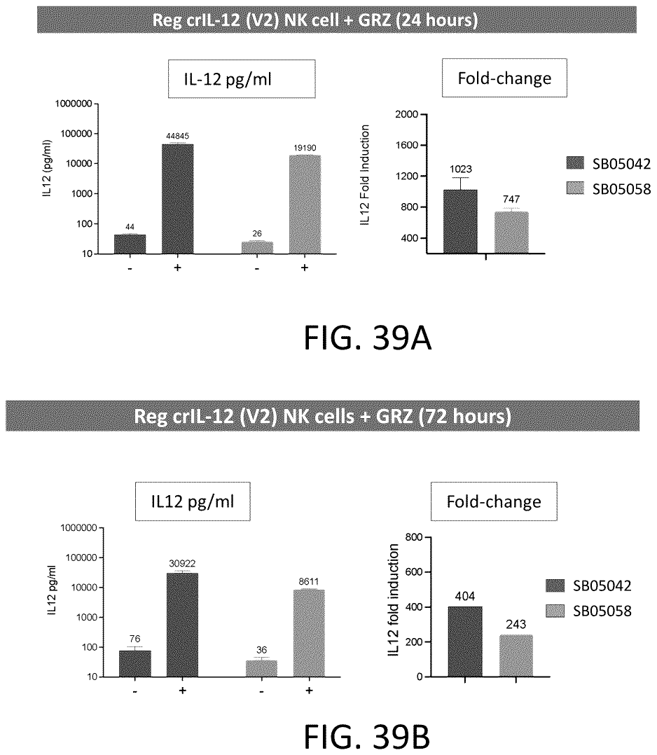

The tested construct candidates resulted in low IL12 basal levels in the absence of GRZ (less than 100 pg/ml) and greater than 100-fold induction of IL12 by 0.1 μM of GRZ (p=<0.0001). FIG. 39A-39B show two different time points (24 hours and 72 hours, respectively) after addition of GRZ to NK cells expressing the SB05042 and SB05058 constructs.

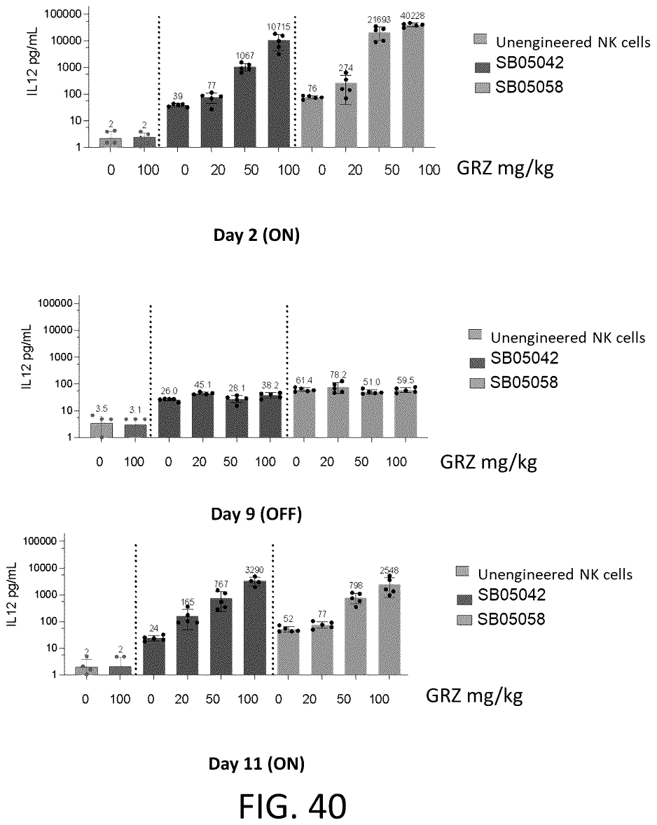

To assess whether the grazoprevir can be used to transition the circuit in an on to off or off to on state in a mouse model, the following study was designed. On day 0, NK cells were injected (IV) in the presence of grazoprevir or vehicle. On days 1, 9, and 10, another dose of grazoprevir or vehicle was injected. Mice were bled on days 2, 9, and 11 to assess expression of IL12. FIG. 40 shows the results of the study. On day 2, IL12 expression increased in the presence of 20, 50, and 100 mg/kg GRZ as compared to the control. On day 9, where GRZ administration has not occurred for 8 days, expression of IL12 is decreased as compared to sampling on day 2. On day 11, expression has increased once again in relation to the control.

### Example 9: Assessment of Co-Transduction of GPC3 CAR/IL15 and Regulated IL12 Constructs

Function and expression of GPC3 CAR, IL15 and IL12 were assessed in NK cells that were co-transduced with GPC/IL15 constructs and the regulated IL12 construct.

### Expression of GPC3 CAR IL15

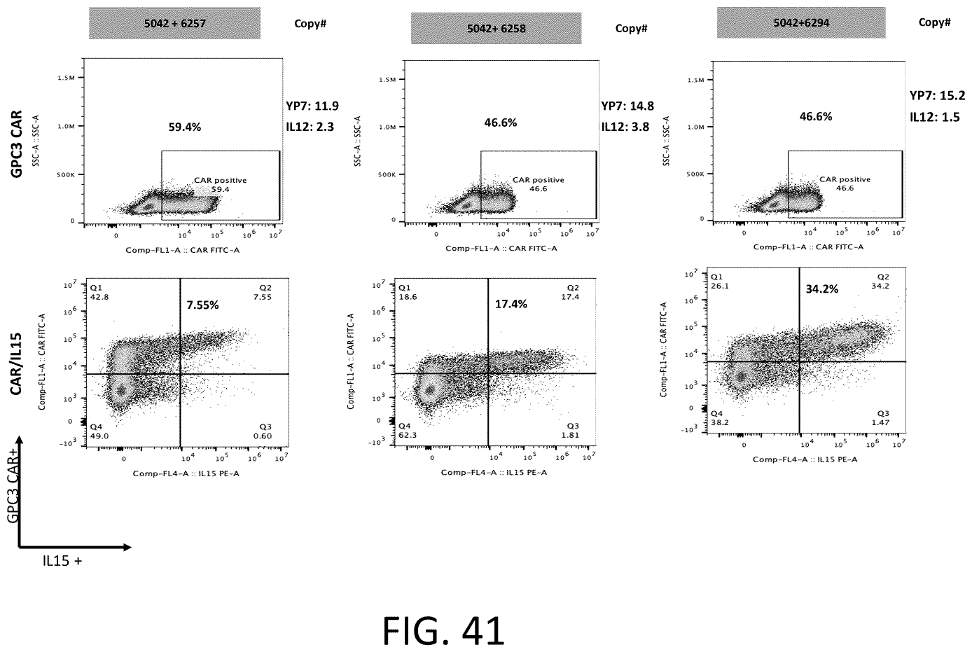

Three construct combinations were tested: 1) SB05042+SB0257, 2) SB05042+SB06258, and 3) SB05042 and SB06294. NK cells co-transduced with SB05042+SB06257 or SB05042+SB06258 expressed GPC3 CAR and IL15 populations and similar copies per cell. NK cells co-transduced with SB06294 exhibited a higher double positive (GPC+/IL15+) population with a slight decrease in CAR only population and with similar copies per cell (FIG. 41)

### Expression of Secreted IL12 and IL15

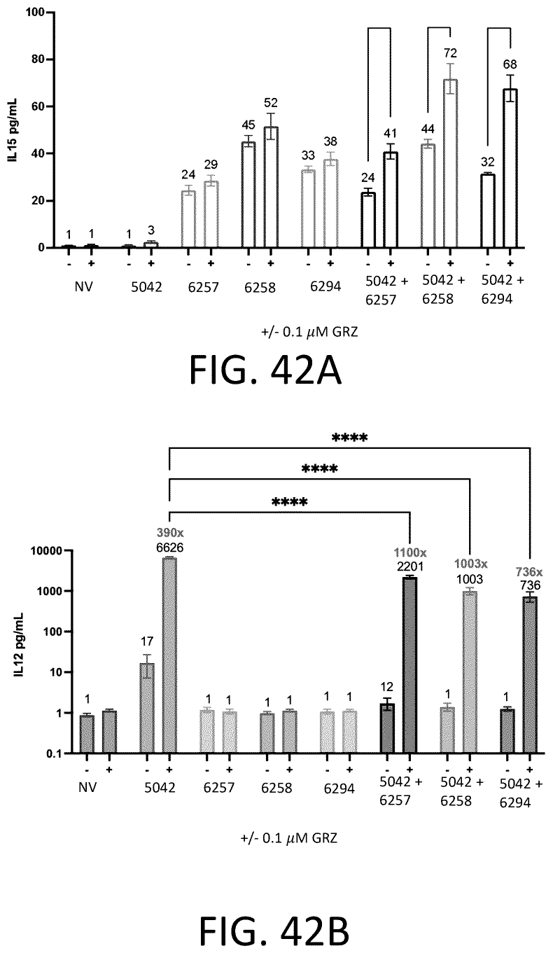

Expression of secreted IL12 and IL15 were measured in NK cells in the presence or absence of grazoprevir was tested. 200,000 transduced NK cells were suspended in 200 μL of NK MACS media supplemented with IL2. Grazoprevir was added to “+” conditions at a molar concentration of 0.1 μM. NK cells were incubated for 48 hours at 37C prior to measurement of the supernatant for IL15 (FIG. 42A) and IL12 (FIG. 42B) concentration. IL15 expression increased slightly in the presence of grazoprevir, with the co-transduced NK cells showing statistically significant IL15 expression in the presence of GRZ. NK cells co-transduced with SB05042+SB06257 expressed 2201 μg/mL IL12 in the presence of grazoprevir, as compared to 12 pg/mL in the absence of grazoprevir (1100-fold induction). SB05042+SB06258 cotransduction exhibited 1003-fold induction in the presence of grazoprevir. SB05042+SB06294 co transduction exhibited 736-fold induction. The three co-transduction combinations were statistically significant compared to NK cells transduced with SB05042 alone. Assessing IL12 expression, NK cells transduced with SB05042 alone showed induction of IL12 in the presence of grazoprevir, showing an 390-fold increase in expression.

### Cytokine Secretion During Serial Killing (Huh7)

Serial killing of target cells were carried out as previously described using NK cells singly transduced or co-transduced with GPC3 CAR/IL15 (SB06257, SB06258, SB06294) and/or IL12 constructs (SB05042).

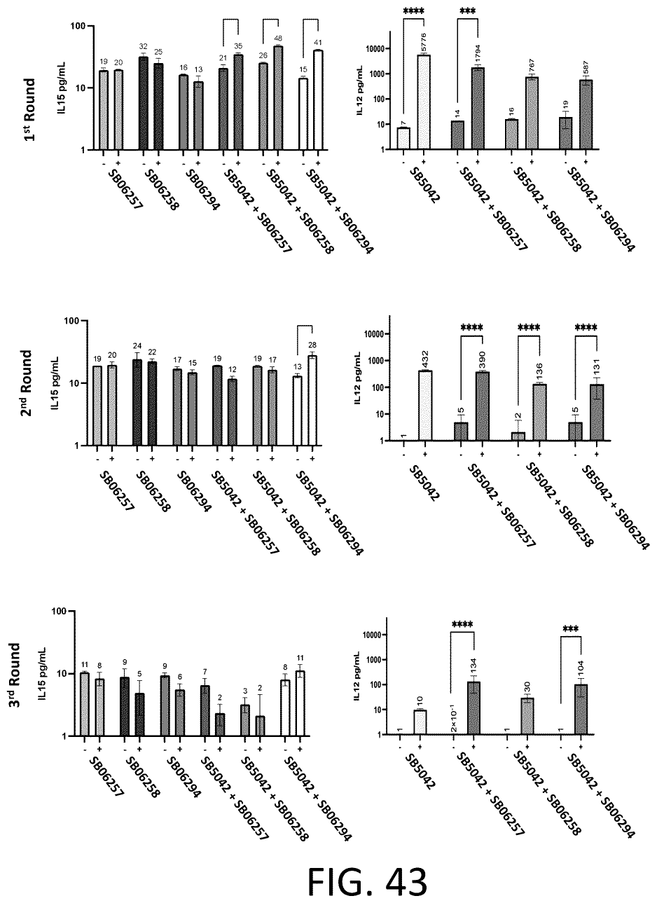

Co-transduced samples maintained low amounts of IL12 induction into the 3rd round in the presence of GRZ. Overall cytokine secretion decreases overtime in both IL12 and IL15 (FIG. 43). In the presence of grazoprevir, SB05042 and SB05042+SB06257 transductions showed significant induction of IL12 expression in the first round of killing. In the second round, the three co-transductions with the different GPC3 CAR expressing constructs (SB06257, SB06258, SB06294) and SB05042 showed statistically significant induction of IL12. In the third round, only SB05042+SB06257 and SB05042+SB06294 showed significant IL12 induction.

Serial Killing Assays with Co-Transduced NK Cells

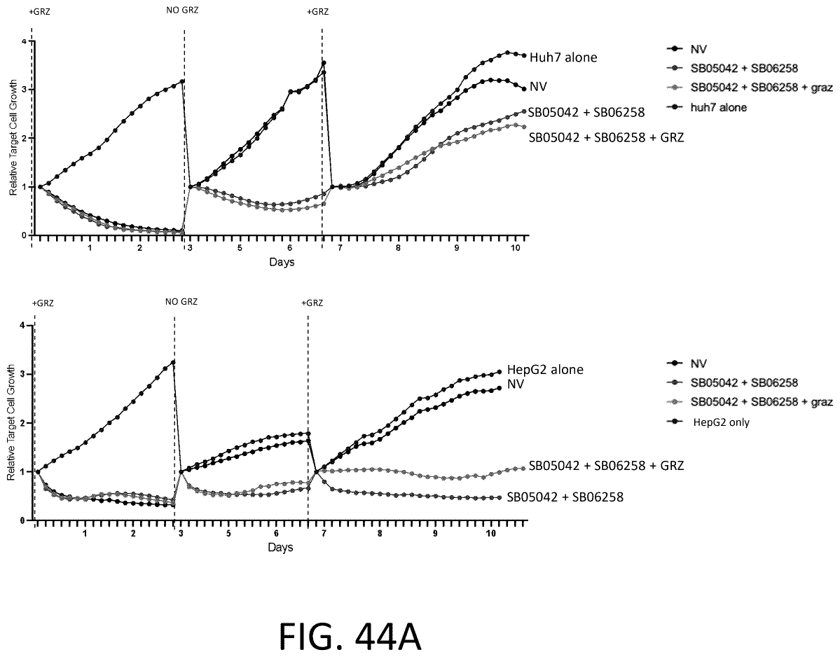

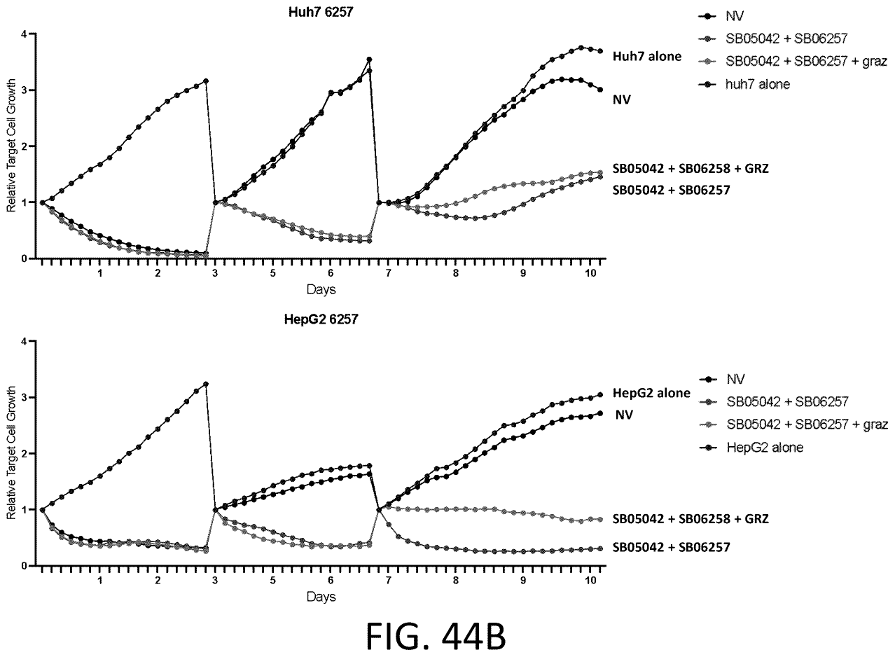

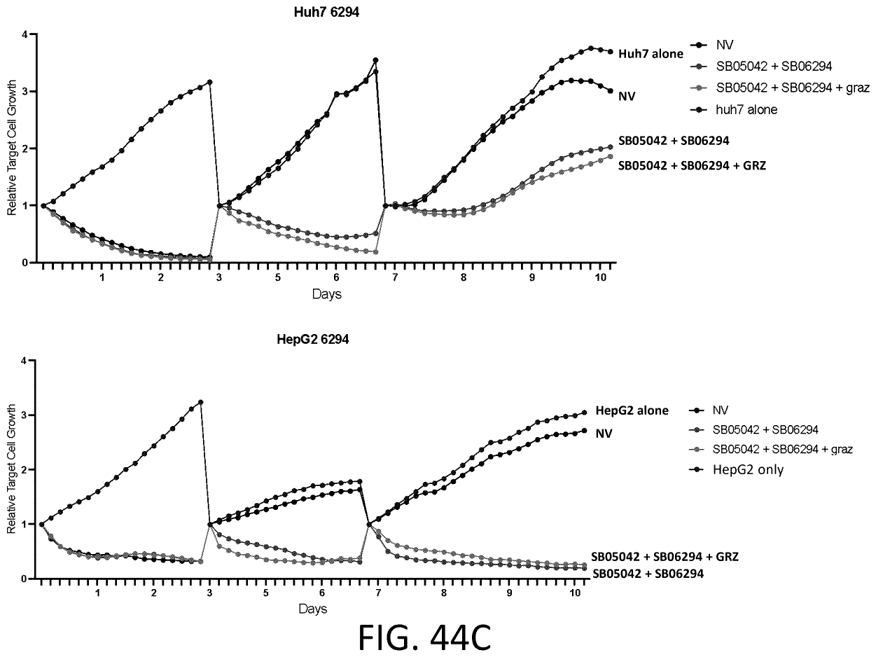

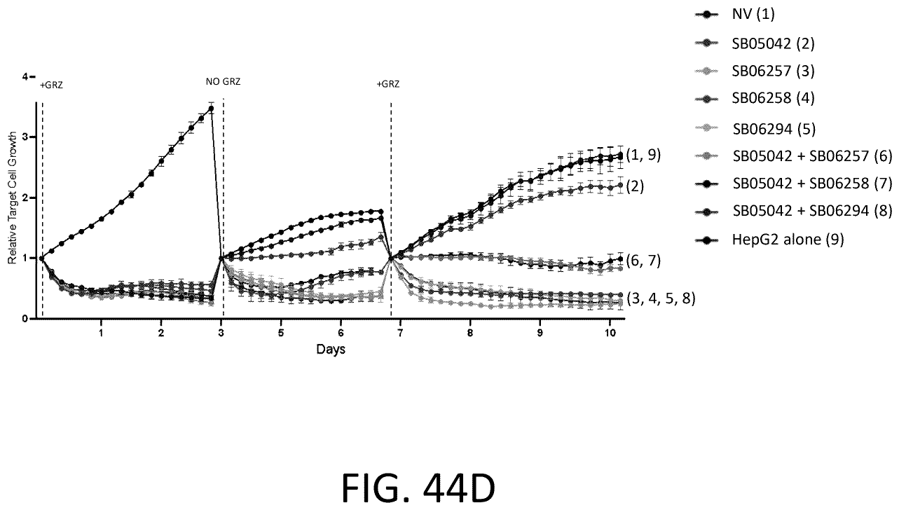

The cell killing effect of NK cells that were co-transduced with GPC3 CAR/IL15 (SB06257, SB06258, SB06294) and/or IL12 constructs (SB05042) were assessed using a serial killing assay. NK cells co-transduced with SB05042+SB06258 (FIG. 44A), SB05042+SB06257 (FIG. 44B) and SB05042+SB06294 (FIG. 44C) were used in a serial killing assay in which GRZ was added at the first and third rounds of cell killing. When co-cultured with HepG2 we see a greater difference between +/−GRZ (induced IL12 or not) as compared to huh7. FIG. 44D shows a combination of the data shown in FIGS. 44A-44C.

### Example 10: Selection of GPC3 CAR/IL15 Clones

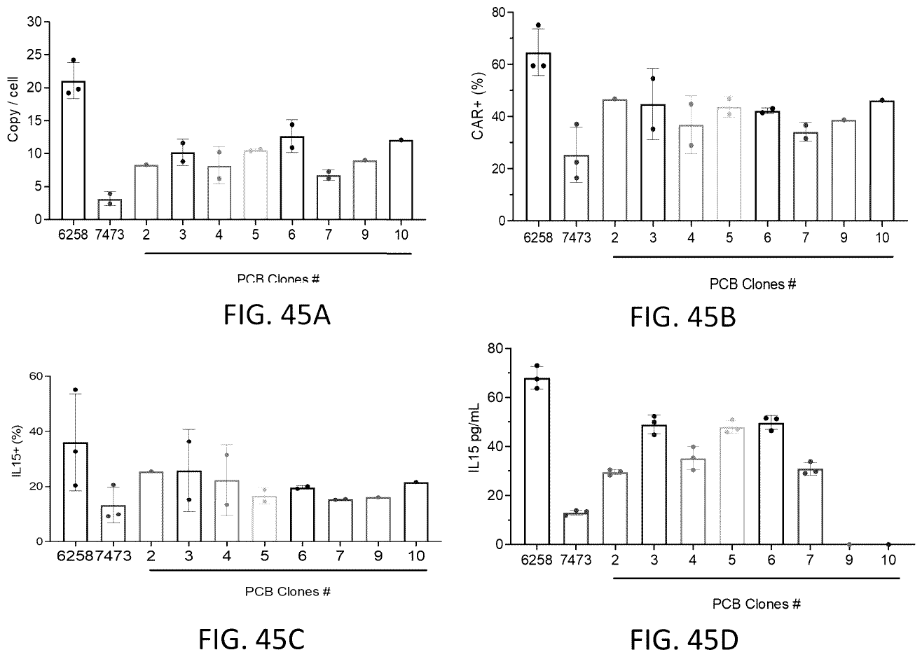

Selection of clones were performed by transducing NK cells that have stably integrated the expression construct. A lower MOI was used (MOI=3) was used for clonal selection of SB06258. A control transient transduction (MOI=15) was also performed used in SB06258 and SB07273 (identical to SB06258 but contains a kanamycin resistance marker instead of an ampicillin resistance marker). 8 days after transduction, the cells were assessed. The copies per cell was lower in the PCB clones as compared to the transient transduction using SB06258 (FIG. 45A). CAR expression was relatively constant across the different PCB clones (FIG. 45B), as well as the IL15+ population (FIG. 45C). Secreted IL15 of PCB clones was measured to be greater than 30 μg/mL (FIG. 45D).

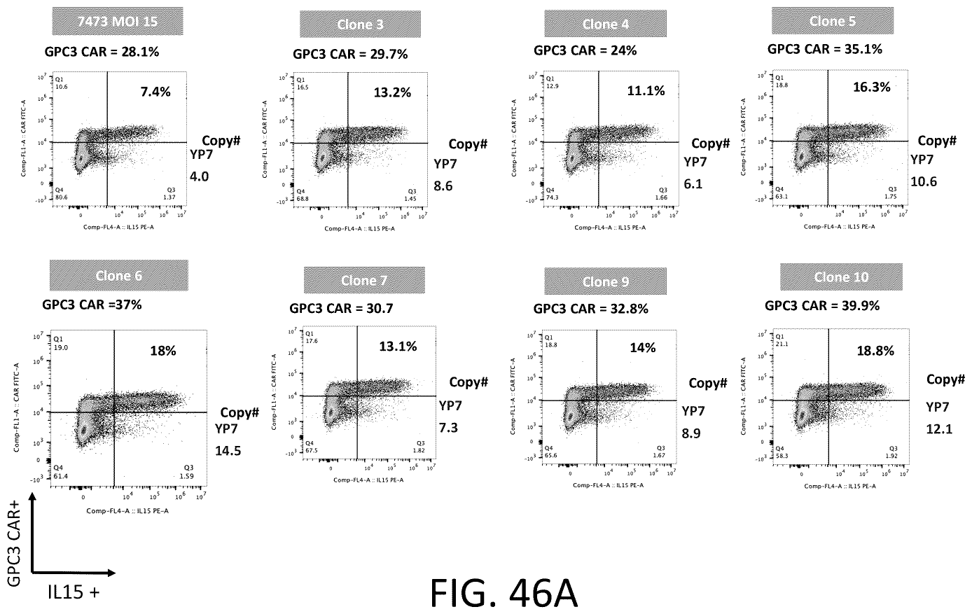

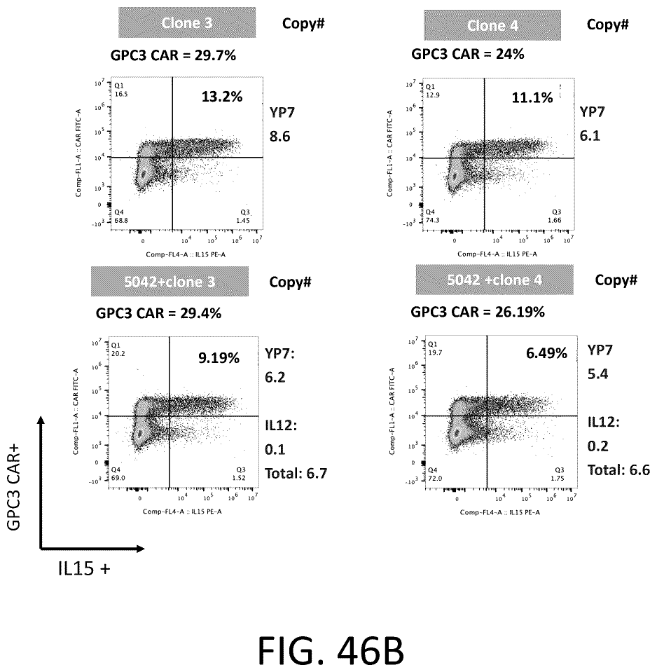

Flow cytometry was also used to assess the expression of the GPC3 CAR and IL15 in the PCM clones. As a control, SB07473 was used to transduced NK cells at an MOI=15. PCB clones were transduced at an MOI of 3.0. For all PCR clones, GPC3 CAR expression was greater than 20% (FIG. 46A).

For select clones, SB05042 was also co-transduced to assess the expression of the GPC3 CAR, membrane bound IL15 and membrane bound IL12 9 days after transduction. Clone 3 (MOI=3.0) and clone 4 (MOI=3.0) was co-transduced with SB05042 (MOI=0.05). During co-transduction, there was similar expression of the GPC3 CAR and membrane bound IL12 (FIG. 46B). Table 19 shows a summary of the expression levels of the PCB clones transduced with SB06258.

TABLE 19

| | | | PCB Clones | | | | | | | |
|---|---|---|---|---|---|---|---|---|---|---|
| | 6258 | 7473 | 2 | 3 | 4 | 5 | 6 | 7 | 9 | 10 |
| Copy # | 19.8 | 3.9 | | 8.88 | 6.2 | 10.7 | 14.4 | 7.9 | 9 | 12.1 |
| | 19.2 | 2.4 | | | | 10.4 | 10.9 | 6.2 | | |
| | 24.2 | | 8.3 | 11.6 | 10.2 | | | | | |
| CAR % | 59.5 | 22.5 | | 35.2 | 29 | 40.9 | 43.1 | 36.7 | 38.8 | 46.3 |
| | 59.5 | 16.5 | | | | 46.8 | 41.5 | 31.6 | | |
| | 75.1 | 37.1 | 46.8 | 54.6 | 44.8 | | | | | |
| memb-IL15 | 32.7 | 9.23 | | 15.2 | 13.4 | 18.8 | 20.1 | 15.4 | 16.1 | 21.6 |
| | 20.4 | 9.9 | | | | 14.6 | 19.2 | 15.2 | | |
| | 55.1 | 20.6 | 25.5 | 36.3 | 31.5 | | | | | |
| Sec-IL15 | 73.0 | 11.9 | 30.6 | 49.9 | 39.9 | 51.0 | 51.5 | 33.8 | | |
| | 63.8 | 13.9 | 29.8 | 44.8 | 30.4 | 45.8 | 46.5 | 29.0 | | |
| | 67.6 | 13.5 | 28.3 | 52.4 | 35.4 | 47.1 | 51.3 | 29.8 | | |

TABLE 20

| SEQ ID NO | Construct | Sequence |
|---|---|---|
| 326 | SB07472 | aagcttggaattcgagcttgcatgcctgcaggtcgttacataacttacggtaaatggcccgcctggctgaccgcccaac |
| | | gacccccgcccattgacgtcaataatgacgtatgttcccatagtaacgccaatagggactttccattgacgtcaatgggt |
| | | ggagtatttacggtaaactgcccacttggcagtacatcaagtgtatcatatgccaagtacgccccctattgacgtcaatg |
| | | acggtaaatggcccgcctggcattatgcccagtacatgaccttatgggactttcctacttggcagtacatctacgtattag |
| | | tcatcgctattaccatggtgatgcggttttggcagtacatcaatgggcgtggatagcggtttgactcacggggatttcca |
| | | agtctccaccccattgacgtcaatgggagtttgttttggcaccaaaatcaacgggactttccaaaatgtcgtaacaactc |
| | | cgccccattgacgcaaatgggcggtaggcgtgtacggtgggaggtctatataagcagagctcaataaaagagccca |
| | | caacccctcactcggcgcgccagtcctccgattgactgagtcgcccgggtacccgtgtatccaataaaccctcttgca |
| | | gttgcatccgacttgtggtctcgctgttccttgggagggtctcctctgagtgattgactacccgtcagcgggggtctttca |
| | | tttgggggctcgtccgagatcgggagacccctgcccagggaccaccgacccaccaccgggaggtaagctggcca |
| | | gcaacttatctgtgtctgtccgattgtctagtgtctatgactgattttatgcgcctgcgtcggtactagttagctaactagctc |
| | | tgtatctggcggacccgtggtggaactgacgagttcggaacacccggccgcaaccctgggagacgtcccagggact |
| | | tcgggggccgtttttgtggcccgacctgagtcctaaaatcccgatcgtttaggactctttggtgcaccccccttagagga |
| | | gggatatgtggttctggtaggagacgagaacctaaaacagttcccgcctccgtctgaatttttgctttcggtttgggacc |
| | | gaagccgcgccgcgcgtcttgtctgctgcagcatcgttctgtgttgtctctgtctgactgtgtttctgtatttgtctgaaaat |
| | | atgggccccccctcgaggtaacgccattttgcaaggcatggaaaaataccaaaccaagaatagagaagttcagatca |
| | | agggcgggtacatgaaaatagctaacgttgggccaaacaggatatctgcggtgagcagtttcggccccggcccggg |
| | | gccaagaacagatggtcaccgcagtttcggccccggcccgaggccaagaacagatggtccccagatatggcccaa |
| | | ccctcagcagtttcttaagacccatcagatgtttccaggctcccccaaggacctgaaatgaccctgcgccttatttgaatt |
| | | aaccaatcagcctgcttctcgcttctgttcgcgcgcttctgcttcccgagctctataaaagagctcacaacccctcactcg |
| | | gcgcgccagtcctccgacagactgagtcgcccgggGCCGCCACCATGCTGCTGCTGGTCAC |
| | | ATCTCTGCTGCTGTGCGAGCTGCCCCATCCTGCCTTTCTGCTGATCCCTC |
| | | ACATGGACATCGTGATGACACAGAGCCCCGATAGCCTGGCCGTGTCTC |
| | | TGGGAGAAAGAGCCACCATCAACTGCAAGAGCAGCCAGAGCCTGCTG |
| | | TACTCCAGCAACCAGAAGAACTACCTGGCCTGGTATCAGCAAAAGCCC |
| | | GGCCAGCCTCCTAAGCTGCTGATCTATTGGGCCAGCTCCAGAGAAAGC |
| | | GGCGTGCCCGATAGATTTTCTGGCTCTGGCAGCGGCACCGACTTCACCC |
| | | TGACAATTTCTAGCCTGCAAGCCGAGGACGTGGCCGTGTACTACTGCC |
| | | AGCAGTACTACAACTACCCTCTGACCTTCGGCCAGGGCACCAAGCTGG |
| | | AAATCAAAGGCGGCGGAGGATCTGGCGGAGGTGGAAGTGGCGGAGGC |
| | | GGATCTGAAGTGCAGCTGGTTGAATCAGGTGGCGGCCTGGTTCAACCT |
| | | GGCGGATCTCTGAGACTGAGCTGTGCCGCCAGCGGCTTCACCTTCAAC |
| | | AAGAACGCCATGAACTGGGTCCGACAGGCCCCTGGCAAAGGCCTTGAA |
| | | TGGGTCGGACGGATCCGGAACAAGACCAACAACTACGCCACCTACTAC |
| | | GCCGACAGCGTGAAGGCCAGATTCACCATCAGCCGGGACGACAGCAA |
| | | GAACAGCCTGTACCTGCAGATGAACTCCCTGAAAACCGAGGACACCGC |
| | | CGTGTATTATTGCGTGGCCGGCAACAGCTTTGCCTACTGGGGACAGGG |
| | | AACCCTGGTCACCGTGTCTGCCACAACAACCCCTGCTCCTAGACCTCCT |
| | | ACACCAGCTCCTACAATCGCCCTGCAGCCTCTGTCTCTGAGGCCAGAA |
| | | GCTTGTAGACCAGCTGCTGGCGGAGCCGTGCATACAAGAGGACTGGAC |
| | | TTCGCCTGTGATGTGGCCGCCATTCTCGGACTGGGACTTGTTCTGGGAC |
| | | TGCTGGGACCTCTGGCCATTCTGCTGGCTCTGTATCTGCTGCGGAGGGA |
| | | CCAAAGACTGCCTCCTGATGCTCACAAGCCTCCAGGCGGAGGCAGCTT |
| | | CAGAACCCCTATCCAAGAGGAACAGGCCGACGCTCACAGCACCCTGGC |
| | | CAAGATTAGAGTGAAGTTCAGCAGAAGCGCCGACGCACCCGCCTATAA |
| | | GCAGGGACAGAACCAGCTGTACAACGAGCTGAACCTGGGGAGAAGAG |
| | | AAGAGTACGACGTGCTGGACAAGCGGAGAGGCAGAGATCCTGAGATG |
| | | GGCGGCAAGCCCAGACGGAAGAATCCTCAAGAGGGCCTGTATAATGA |
| | | GCTGCAGAAAGACAAGATGGCCGAGGCCTACAGCGAGATCGGAATGA |
| | | AGGGCGAGCGCAGAAGAGGCAAGGGACACGATGGACTGTACCAGGGC |
| | | CTGAGCACCGCCACCAAGGATACCTATGATGCCCTGCACATGCAGGCC |
| | | CTGCCTCCAAGAGGTAGCGGCCAGTGTACCAACTACGCCCTGCTGAAA |
| | | CTGGCCGGCGACGTGGAATCTAATCCTGGACCTGGATCTGGCGAGGGA |
| | | CGCGGGAGTCTACTGACGTGTGGAGACGTGGAGGAAAACCCTGGACCT |
| | | ATGGACTGGACCTGGATCCTGTTTCTGGTGGCCGCTGCCACAAGAGTG |
| | | CACAGCAATTGGGTCAACGTGATCAGCGACCTGAAGAAGATCGAGGA |
| | | CCTGATCCAGAGCATGCACATCGACGCCACACTGTACACCGAGAGCGA |
| | | CGTGCACCCTAGCTGTAAAGTGACCGCCATGAAGTGCTTTCTGCTGGA |
| | | ACTGCAAGTGATCAGCCTGGAAAGCGGCGACGCCAGCATCCACGACAC |
| | | CGTGGAAAACCTGATCATCCTGGCCAACAACAGCCTGAGCAGCAACGG |
| | | CAATGTGACCGAGTCCGGCTGCAAAGAGTGCGAGGAACTGGAAGAGA |
| | | AGAATATCAAAGAGTTCCTGCAGAGCTTCGTGCACATCGTGCAGATGT |
| | | TCATCAACACAAGCTCTGGCGGCGGAGGATCTGGCGGAGGTGGAAGC |
| | | GGAGTTACACCCGAGCCTATCTTCAGCCTGATCGGAGGCGGTAGCGGA |
| | | GGCGGAGGAAGTGGTGGCGGATCTCTGCAACTGCTGCCTAGCTGGGCC |
| | | ATCACACTGATCTCCGTGAACGGCATCTTCGTGATCTGCTGCCTGACCT |
| | | ACTGCTTCGCCCCTAGATGCAGAGAGCGGCGGAGAAACGAACGGCTG |
| | | AGAAGAGAATCTGTGCGGCCCGTTtaaggatccggattagtccaatttgttaaagacaggatgg |
| | | gctgcaggaattccgataatcaacctctggattacaaaatttgtgaaagattgactggtattcttaactatgttgctcctttta |
| | | cgctatgtggatacgctgctttaatgcctttgtatcatgctattgcttcccgtatggctttcattttctcctccttgtataaatcc |
| | | tggttgctgtctctttatgaggagttgtggcccgttgtcaggcaacgtggcgtggtgtgcactgtgtttgctgacgcaacc |
| | | cccactggttggggcattgccaccacctgtcagctcctttccgggactttcgctttccccctccctattgccacggcgga |
| | | actcatcgccgcctgccttgcccgctgctggacaggggctcggctgttgggcactgacaattccgtggtgttgtcggg |
| | | gaagctgacgtcctttccatggctgctcgcctgtgttgccacctggattctgcgcgggacgtccttctgctacgtcccttc |
| | | ggccctcaatccagcggaccttccttcccgcggcctgctgccggctctgcggcctcttccgcgtcttcgccttcgccct |
| | | cagacgagtcggatctccctttgggccgcctccccgcctggagaattcgatatcagtggtccaggctctagttttgactc |
| | | aacaatatcaccagctgaagcctatagagtacgagccatagataaaataaaagattttatttagtctccagaaaaaggg |
| | | gggaatgaaagaccccacctgtaggtttggcaagctagcaataaaagagcccacaacccctcactcggggcgccag |
| | | tcctccgattgactgagtcgcccggccgcttcgagcagacatgataagatacattgatgagtttggacaaaccacaact |
| | | agaatgcagtgaaaaaaatgctttatttgtgaaatttgtgatgctattgctttatttgtaaccattataagctgcaataaacaa |
| | | gttaacaacaacaattgcattcattttatgtttcaggttcagggggagatgtgggaggttttttaaagcaagtaaaacctct |
| | | acaaatgtggtaaaatcgataaggatcgggtacccgtgtatccaataaaccctcttgcagttgcatccgacttgtggtct |
| | | cgctgttccttgggagggtctcctctgagtgattgactacccgtcagcgggggtctttcacacatgcagcatgtatcaaa |
| | | attaatttggttttttttcttaagctgtgccttctagttgccagccatctgttgtttgcccctcccccgtgccttccttgaccctg |
| | | gaaggtgccactcccactgtcctttcctaataaaatgaggaaattgcatcgcattgtctgagtaggtgtcattctattctgg |
| | | ggggggggtggggcaggacagcaagggggaggattgggaagacaatagcaggcatgctggggatgcggtggg |
| | | ctctatggagatcccgcggtacctcgcgaatgcatctagatccaatggcctttttggcccagacatgataagatacattg |
| | | atgagtttggacaaaccacaactagaatgcagtgaaaaaaatgctttatttgtgaaatttgtgatgctattgctttatttgtaa |
| | | ccattataagctgcaataaacaagttgcggccgcttagccctcccacacataaccagagggcagcaattcacgaatcc |
| | | caactgccgtcggctgtccatcactgtccttcactatggctttgatcccaggatgcagatcgagaagcacctgtcggca |
| | | ccgtccgcaggggctcaagatgcccctgttctcatttccgatcgcgacgatacaagtcaggttgccagctgccgcagc |
| | | agcagcagtgcccagcaccacgagttctgcacaaggtcccccagtaaaatgatatacattgacaccagtgaagatgc |
| | | ggccgtcgctagagagagctgcgctggcgacgctgtagtcttcagagatggggatgctgttgattgtagccgttgctct |
| | | ttcaatgagggtggattcttcttgagacaaaggcttggccatgcggccgccgctcggtgttcgaggccacacgcgtca |
| | | ccttaatatgcgaagtggacctcggaccgcgccgccccgactgcatctgcgtgttcgaattcgccaatgacaagacg |
| | | ctgggcggggtttgtgtcatcatagaactaaagacatgcaaatatatttcttccggggggtaccggcctttttggccAT |
| | | TGGatcggatctggccaaaaaggcccttaagtatttacattaaatggccatagtacttaaagttacattggcttccttga |
| | | aataaacatggagtattcagaatgtgtcataaatatttctaattttaagatagtatctccattggctttctactttttcttttattttt |
| | | ttttgtcctctgtcttccatttgttgttgttgttgtttgtttgtttgtttgttggttggttggttaatttttttttaaagatcctacactat |
| | | agttcaagctagactattagctactctgtaacccagggtgaccttgaagtcatgggtagcctgctgttttagccttcccac |
| | | atctaagattacaggtatgagctatcatttttggtatattgattgattgattgattgatgtgtgtgtgtgtgattgtgtttgtgtgt |
| | | gtgaTtgtgTaTatgtgtgtatggTtgtgtgtgtgtgtgtgtgtgtgtgtgtgtgtgtgtgtgtgtgtgtgttgtgTaTa |
| | | TatatttatggtagtgagagGcaacgctccggctcaggtgtcaggttggtttttgagacagagtctttcacttagcttgg |
| | | aattcactggccgtcgttttacaacgtcgtgactgggaaaaccctggcgttacccaacttaatcgccttgcagcacatcc |
| | | ccctttcgccagctggcgtaatagcgaagaggcccgcaccgatcgcccttcccaacagttgcgcagcctgaatggcg |
| | | aatggcgcctgatgcggtattttctccttacgcatctgtgcggtatttcacaccgcatatggtgcactctcagtacaatctg |
| | | ctctgatgccgcatagttaagccagccccgacacccgccaacacccgctgacgcgccctgacgggcttgtctgctcc |
| | | cggcatccgcttacagacaagctgtgaccgtctccgggagctgcatgtgtcagaggttttcaccgtcatcaccgaaac |
| | | gcgcgagacgaaagggcctcgtgatacgcctatttttataggttaatgtcatgataataatggtttcttagacgtcaggtg |
| | | gcacttttcggggaaatgtgcgcggaacccctatttgtttatttttctaaatacattcaaatatgtatccgctcatgagacaa |
| | | taaccctgataaatgcttcaataatattgaaaaaggaagagtatgagccatattcaacgggaaacgtcgaggccgcga |
| | | ttaaattccaacatggatgctgatttatatgggtataaatgggctcgcgataatgtcgggcaatcaggtgcgacaatctat |
| | | cgcttgtatgggaagcccgatgcgccagagttgtttctgaaacatggcaaaggtagcgttgccaatgatgttacagatg |
| | | agatggtcagactaaactggctgacggaatttatgcctcttccgaccatcaagcattttatccgtactcctgatgatgcat |
| | | ggttactcaccactgcgatccccggaaaaacagcattccaggtattagaagaatatcctgattcaggtgaaaatattgtt |
| | | gatgcgctggcagtgttcctgcgccggttgcattcgattcctgtttgtaattgtccttttaacagcgatcgcgtatttcgtct |
| | | cgctcaggcgcaatcacgaatgaataacggtttggttgatgcgagtgattttgatgacgagcgtaatggctggcctgtt |
| | | gaacaagtctggaaagaaatgcataaacttttgccattctcaccggattcagtcgtcactcatggtgatttctcacttgata |
| | | accttatttttgacgaggggaaattaataggttgtattgatgttggacgagtcggaatcgcagaccgataccaggatctt |
| | | gccatcctatggaactgcctcggtgagttttctccttcattacagaaacggctttttcaaaaatatggtattgataatcctga |
| | | tatgaataaattgcagtttcatttgatgctcgatgagtttttctaactgtcagaccaagtttactcatatatactttagattgattt |
| | | aaaacttcatttttaatttaaaaggatctaggtgaagatcctttttgataatctcatgaccaaaatcccttaacgtgagttttc |
| | | gttccactgagcgtcagaccccgtagaaaagatcaaaggatcttcttgagatcctttttttctgcgcgtaatctgctgcttg |
| | | caaacaaaaaaaccaccgctaccagcggtggtttgtttgccggatcaagagctaccaactctttttccgaaggtaactg |
| | | gcttcagcagagcgcagataccaaatactgttcttctagtgtagccgtagttaggccaccacttcaagaactctgtagca |
| | | ccgcctacatacctcgctctgctaatcctgttaccagtggctgctgccagtggcgataagtcgtgtcttaccgggttgga |
| | | ctcaagacgatagttaccggataaggcgcagcggtcgggctgaacggggggttcgtgcacacagcccagcttgga |
| | | gcgaacgacctacaccgaactgagatacctacagcgtgagctatgagaaagcgccacgcttcccgaagggagaaa |
| | | ggcggacaggtatccggtaagcggcagggtcggaacaggagagcgcacgagggagcttccagggggaaacgcc |
| | | tggtatctttatagtcctgtcgggtttcgccacctctgacttgagcgtcgatttttgtgatgctcgtcaggggggcggagc |
| | | ctatggaaaaacgccagcaacgcggcctttttacggttcctggccttttgctggccttttgctcacatgttctttcctgcgtt |
| | | atcccctgattctgtggataaccgtattaccgcctttgagtgagctgataccgctcgccgcagccgaacgaccgagcg |
| | | cagcgagtcagtgagcgaggaagcggaagagcgcccaatacgcaaaccgcctctccccgcgcgttggccgattca |
| | | ttaatgcagctggcacgacaggtttcccgactggaaagcgggcagtgagcgcaacgcaattaatgtgagttagctca |
| | | ctcattaggcaccccaggctttacactttatgcttccggctcgtatgttgtgtggaattgtgagcggataacaatttcacac |
| | | aggaaacagctatgaccatgattacgcc |
| 327 | SB07473 | aagcttgGaattcgagcttgcatgcctgcaggtcgttacataacttacggtaaatggcccgcctggctgaccgcccaa |
| | | cgacccccgcccattgacgtcaataatgacgtatgttcccatagtaacgccaatagggactttccattgacgtcaatgg |
| | | gtggagtatttacggtaaactgcccacttggcagtacatcaagtgtatcatatgccaagtacgccccctattgacgtcaa |
| | | tgacggtaaatggcccgcctggcattatgcccagtacatgaccttatgggactttcctacttggcagtacatctacgtatt |
| | | agtcatcgctattaccatggtgatgcggttttggcagtacatcaatgggcgtggatagcggtttgactcacggggatttc |
| | | caagtctccaccccattgacgtcaatgggagtttgttttggcaccaaaatcaacgggactttccaaaatgtcgtaacaac |
| | | tccgccccattgacgcaaatgggcggtaggcgtgtacggtgggaggtctatataagcagagctcaataaaagagcc |
| | | cacaacccctcactcggcgcgccagtcctccgattgactgagtcgcccgggtacccgtgtatccaataaaccctcttg |
| | | cagttgcatccgacttgtggtctcgctgttccttgggagggtctcctctgagtgattgactacccgtcagcgggggtcttt |
| | | catttgggggctcgtccgagatcgggagacccctgcccagggaccaccgacccaccaccgggaggtaagctggc |
| | | cagcaacttatctgtgtctgtccgattgtctagtgtctatgactgattttatgcgcctgcgtcggtactagttagctaactag |
| | | ctctgtatctggcggacccgtggtggaactgacgagttcggaacacccggccgcaaccctgggagacgtcccagg |
| | | gacttcgggggccgtttttgtggcccgacctgagtcctaaaatcccgatcgtttaggactctttggtgcaccccccttag |
| | | aggagggatatgtggttctggtaggagacgagaacctaaaacagttcccgcctccgtctgaatttttgctttcggtttgg |
| | | gaccgaagccgcgccgcgcgtcttgtctgctgcagcatcgttctgtgttgtctctgtctgactgtgtttctgtatttgtctg |
| | | aaaatatgggccccccctcgaggtaacgccattttgcaaggcatggaaaaataccaaaccaagaatagagaagttca |
| | | gatcaagggcgggtacatgaaaatagctaacgttgggccaaacaggatatctgcggtgagcagtttcggccccggc |
| | | ccggggccaagaacagatggtcaccgcagtttcggccccggcccgaggccaagaacagatggtccccagatatgg |
| | | cccaaccctcagcagtttcttaagacccatcagatgtttccaggctcccccaaggacctgaaatgaccctgcgccttatt |
| | | tgaattaaccaatcagcctgcttctcgcttctgttcgcgcgcttctgcttcccgagctctataaaagagctcacaacccct |
| | | cactcggcgcgccagtcctccgacagactgagtcgcccgggGCCGCCACCATGCTGCTGCTGG |
| | | TCACATCTCTGCTGCTGTGCGAGCTGCCCCATCCTGCCTTTCTGCTGAT |
| | | CCCTCACATGGAAGTGCAGCTGGTGGAATCTGGCGGAGGACTGGTTCA |
| | | ACCTGGCGGCTCTCTGAGACTGTCTTGTGCCGCCAGCGGCTTCACCTTC |
| | | AACAAGAACGCCATGAACTGGGTCCGACAGGCCCCTGGCAAAGGCCT |
| | | TGAATGGGTCGGACGGATCCGGAACAAGACCAACAACTACGCCACCT |
| | | ACTACGCCGACAGCGTGAAGGCCAGGTTCACCATCTCCAGAGATGACA |
| | | GCAAGAACAGCCTGTACCTGCAGATGAACTCCCTGAAAACCGAGGAC |
| | | ACCGCCGTGTACTATTGCGTGGCCGGCAATAGCTTTGCCTACTGGGGA |
| | | CAGGGCACCCTGGTTACAGTTTCTGCTGGCGGCGGAGGAAGCGGAGGC |
| | | GGAGGATCCGGTGGTGGTGGATCTGACATCGTGATGACACAGAGCCCC |
| | | GATAGCCTGGCCGTGTCTCTGGGAGAAAGAGCCACCATCAACTGCAAG |
| | | AGCAGCCAGAGCCTGCTGTACTCCAGCAACCAGAAGAACTACCTGGCC |
| | | TGGTATCAGCAAAAGCCCGGCCAGCCTCCTAAGCTGCTGATCTATTGG |
| | | GCCAGCTCCAGAGAAAGCGGCGTGCCCGATAGATTTTCTGGCTCTGGC |
| | | AGCGGCACCGACTTCACCCTGACAATTTCTAGCCTGCAAGCCGAGGAC |
| | | GTGGCCGTGTATTACTGCCAGCAGTACTACAACTACCCTCTGACCTTCG |
| | | GCCAGGGCACCAAGCTGGAAATCAAATCTGGCGCCCTGAGCAACAGC |
| | | ATCATGTACTTCAGCCACTTCGTGCCCGTGTTTCTGCCCGCCAAGCCTA |
| | | CAACAACCCCTGCTCCTAGACCTCCTACACCAGCTCCTACAATCGCCA |
| | | GCCAGCCTCTGTCTCTGAGGCCAGAAGCTTGTAGACCTGCTGCAGGCG |
| | | GAGCCGTGCATACAAGAGGACTGGATTTCGCCTGCGACATCTACATCT |
| | | GGGCCCCTCTGGCTGGAACATGTGGTGTCCTGCTGCTGAGCCTGGTCA |
| | | TCACCCTGTACTGCAACCACCGGCGGAGCAAGAGAAGCAGACTGCTGC |
| | | ACAGCGACTACATGAACATGACCCCTAGACGGCCCGGACCTACCAGA |
| | | AAGCACTACCAGCCTTACGCTCCTCCTAGAGACTTCGCCGCCTACCGG |
| | | TCCAGAGTGAAGTTCAGCAGATCCGCCGATGCTCCCGCCTATCAGCAG |
| | | GGACAGAACCAGCTGTACAACGAGCTGAACCTGGGGAGAAGAGAAGA |
| | | GTACGACGTGCTGGACAAGCGGAGAGGCAGAGATCCTGAGATGGGCG |
| | | GCAAGCCCAGACGGAAGAATCCTCAAGAGGGCCTGTATAATGAGCTG |
| | | CAGAAAGACAAGATGGCCGAGGCCTACAGCGAGATCGGAATGAAGGG |
| | | CGAGCGCAGAAGAGGCAAGGGACACGATGGACTGTACCAGGGCCTGA |
| | | GCACCGCCACCAAGGATACCTATGATGCCCTGCACATGCAGGCCCTGC |
| | | CTCCAAGAGGTAGCGGCCAGTGTACCAACTACGCCCTGCTGAAACTGG |
| | | CCGGCGACGTGGAATCTAATCCTGGACCTGGATCTGGCGAGGGACGCG |
| | | GGAGTCTACTGACGTGTGGAGACGTGGAGGAAAACCCTGGACCTATG |
| | | GACTGGACCTGGATCCTGTTTCTGGTGGCCGCTGCCACAAGAGTGCAC |
| | | AGCAATTGGGTCAACGTGATCAGCGACCTGAAGAAGATCGAGGACCT |
| | | GATCCAGAGCATGCACATCGACGCCACACTGTACACCGAGAGCGACGT |
| | | GCACCCTAGCTGTAAAGTGACCGCCATGAAGTGCTTTCTGCTGGAACT |
| | | GCAAGTGATCAGCCTGGAAAGCGGCGACGCCAGCATCCACGACACCG |
| | | TGGAAAACCTGATCATCCTGGCCAACAACAGCCTGAGCAGCAACGGC |
| | | AATGTGACCGAGTCCGGCTGCAAAGAGTGCGAGGAACTGGAAGAGAA |
| | | GAATATCAAAGAGTTCCTGCAGAGCTTCGTGCACATCGTGCAGATGTT |
| | | CATCAACACAAGCTCTGGCGGCGGAGGATCTGGCGGAGGTGGAAGCG |
| | | GAGTTACACCCGAGCCTATCTTCAGCCTGATCGGAGGCGGTAGCGGAG |
| | | GCGGAGGAAGTGGTGGCGGATCTCTGCAACTGCTGCCTAGCTGGGCCA |
| | | TCACACTGATCTCCGTGAACGGCATCTTCGTGATCTGCTGCCTGACCTA |
| | | CTGCTTCGCCCCTAGATGCAGAGAGCGGCGGAGAAACGAACGGCTGA |
| | | GAAGAGAATCTGTGCGGCCCGTTtaaggatccggattagtccaatttgttaaagacaggatggg |
| | | ctgcaggaattccgataatcaacctctggattacaaaatttgtgaaagattgactggtattcttaactatgttgctccttttac |
| | | gctatgtggatacgctgctttaatgcctttgtatcatgctattgcttcccgtatggctttcattttctcctccttgtataaatcct |
| | | ggttgctgtctctttatgaggagttgtggcccgttgtcaggcaacgtggcgtggtgtgcactgtgtttgctgacgcaacc |
| | | cccactggttggggcattgccaccacctgtcagctcctttccgggactttcgctttccccctccctattgccacggcgga |
| | | actcatcgccgcctgccttgcccgctgctggacaggggctcggctgttgggcactgacaattccgtggtgttgtcggg |
| | | gaagctgacgtcctttccatggctgctcgcctgtgttgccacctggattctgcgcgggacgtccttctgctacgtcccttc |
| | | ggccctcaatccagcggaccttccttcccgcggcctgctgccggctctgcggcctcttccgcgtcttcgccttcgccct |
| | | cagacgagtcggatctccctttgggccgcctccccgcctggagaattcgatatcagtggtccaggctctagttttgact |
| | | caacaatatcaccagctgaagcctatagagtacgagccatagataaaataaaagattttatttagtctccagaaaaagg |
| | | ggggaatgaaagaccccacctgtaggtttggcaagctagcaataaaagagcccacaacccctcactcggggcgcc |
| | | agtcctccgattgactgagtcgcccggccgcttcgagcagacatgataagatacattgatgagtttggacaaaccaca |
| | | actagaatgcagtgaaaaaaatgctttatttgtgaaatttgtgatgctattgctttatttgtaaccattataagctgcaataaa |
| | | caagttaacaacaacaattgcattcattttatgtttcaggttcagggggagatgtgggaggttttttaaagcaagtaaaac |
| | | ctctacaaatgtggtaaaatcgataaggatcgggtacccgtgtatccaataaaccctcttgcagttgcatccgacttgtg |
| | | gtctcgctgttccttgggagggtctcctctgagtgattgactacccgtcagcgggggtctttcacacatgcagcatgtat |
| | | caaaattaatttggttttttttcttaagctgtgccttctagttgccagccatctgttgtttgcccctcccccgtgccttccttgac |
| | | cctggaaggtgccactcccactgtcctttcctaataaaatgaggaaattgcatcgcattgtctgagtaggtgtcattctatt |
| | | ctggggggggggtggggcaggacagcaagggggaggattgggaagacaatagcaggcatgctggggatgcgg |
| | | tgggctctatggagatcccgcggtacctcgcgaatgcatctagatccaatggcctttttggcccagacatgataagata |
| | | cattgatgagtttggacaaaccacaactagaatgcagtgaaaaaaatgctttatttgtgaaatttgtgatgctattgctttat |
| | | ttgtaaccattataagctgcaataaacaagttgcggccgcttagccctcccacacataaccagagggcagcaattcac |
| | | gaatcccaactgccgtcggctgtccatcactgtccttcactatggctttgatcccaggatgcagatcgagaagcacctg |
| | | tcggcaccgtccgcaggggctcaagatgcccctgttctcatttccgatcgcgacgatacaagtcaggttgccagctgc |
| | | cgcagcagcagcagtgcccagcaccacgagttctgcacaaggtcccccagtaaaatgatatacattgacaccagtga |
| | | agatgcggccgtcgctagagagagctgcgctggcgacgctgtagtcttcagagatggggatgctgttgattgtagcc |
| | | gttgctctttcaatgagggtggattcttcttgagacaaaggcttggccatgcggccgccgctcggtgttcgaggccaca |
| | | cgcgtcaccttaatatgcgaagtggacctcggaccgcgccgccccgactgcatctgcgtgttcgaattcgccaatgac |
| | | aagacgctgggcggggtttgtgtcatcatagaactaaagacatgcaaatatatttcttccggggggtaccggcctttttg |
| | | gccATTGGatcggatctggccaaaaaggcccttaagtatttacattaaatggccatagtacttaaagttacattggct |
| | | tccttgaaataaacatggagtattcagaatgtgtcataaatatttctaattttaagatagtatctccattggctttctactttttct |
| | | tttatttttttttgtcctctgtcttccatttgttgttgttgttgtttgtttgtttgtttgttggttggttggttaatttttttttaaagatcct |
| | | acactatagttcaagctagactattagctactctgtaacccagggtgaccttgaagtcatgggtagcctgctgttttagcc |
| | | ttcccacatctaagattacaggtatgagctatcatttttggtatattgattgattgattgattgatgtgtgtgtgtgtgattgtgt |
| | | ttgtgtgtgtgaTtgtgTaTatgtgtgtatggTtgtgtgtgtgtgtgtgtgtgtgtgtgtgtgtgtgttgtgTaTaTatat |
| | | ttatggtagtgagagGcaacgctccggctcaggtgtcaggttggtttttgagacagagtctttcacttagcttggaattc |
| | | actggccgtcgttttacaacgtcgtgactgggaaaaccctggcgttacccaacttaatcgccttgcagcacatccccctt |
| | | tcgccagctggcgtaatagcgaagaggcccgcaccgatcgcccttcccaacagttgcgcagcctgaatggcgaatg |
| | | gcgcctgatgcggtattttctccttacgcatctgtgcggtatttcacaccgcatatggtgcactctcagtacaatctgctct |
| | | gatgccgcatagttaagccagccccgacacccgccaacacccgctgacgcgccctgacgggcttgtctgctcccgg |
| | | catccgcttacagacaagctgtgaccgtctccgggagctgcatgtgtcagaggttttcaccgtcatcaccgaaacgcg |
| | | cgagacgaaagggcctcgtgatacgcctatttttataggttaatgtcatgataataatggtttcttagacgtcaggtggca |
| | | cttttcggggaaatgtgcgcggaacccctatttgtttatttttctaaatacattcaaatatgtatccgctcatgagacaataa |
| | | ccctgataaatgcttcaataatattgaaaaaggaagagtatgagccatattcaacgggaaacgtcgaggccgcgatta |
| | | aattccaacatggatgctgatttatatgggtataaatgggctcgcgataatgtcgggcaatcaggtgcgacaatctatcg |
| | | cttgtatgggaagcccgatgcgccagagttgtttctgaaacatggcaaaggtagcgttgccaatgatgttacagatgag |
| | | atggtcagactaaactggctgacggaatttatgcctcttccgaccatcaagcattttatccgtactcctgatgatgcatgg |
| | | ttactcaccactgcgatccccggaaaaacagcattccaggtattagaagaatatcctgattcaggtgaaaatattgttgat |
| | | gcgctggcagtgttcctgcgccggttgcattcgattcctgtttgtaattgtccttttaacagcgatcgcgtatttcgtctcgc |
| | | tcaggcgcaatcacgaatgaataacggtttggttgatgcgagtgattttgatgacgagcgtaatggctggcctgttgaa |
| | | caagtctggaaagaaatgcataaacttttgccattctcaccggattcagtcgtcactcatggtgatttctcacttgataacc |
| | | ttatttttgacgaggggaaattaataggttgtattgatgttggacgagtcggaatcgcagaccgataccaggatcttgcc |
| | | atcctatggaactgcctcggtgagttttctccttcattacagaaacggctttttcaaaaatatggtattgataatcctgatatg |
| | | aataaattgcagtttcatttgatgctcgatgagtttttctaactgtcagaccaagtttactcatatatactttagattgatttaaa |
| | | acttcatttttaatttaaaaggatctaggtgaagatcctttttgataatctcatgaccaaaatcccttaacgtgagttttcgttc |
| | | cactgagcgtcagaccccgtagaaaagatcaaaggatcttcttgagatcctttttttctgcgcgtaatctgctgcttgcaa |
| | | acaaaaaaaccaccgctaccagcggtggtttgtttgccggatcaagagctaccaactctttttccgaaggtaactggctt |
| | | cagcagagcgcagataccaaatactgtTcttctagtgtagccgtagttaggccaccacttcaagaactctgtagcacc |
| | | gcctacatacctcgctctgctaatcctgttaccagtggctgctgccagtggcgataagtcgtgtcttaccgggttggact |
| | | caagacgatagttaccggataaggcgcagcggtcgggctgaacggggggttcgtgcacacagcccagcttggagc |
| | | gaacgacctacaccgaactgagatacctacagcgtgagctatgagaaagcgccacgcttcccgaagggagaaagg |
| | | cggacaggtatccggtaagcggcagggtcggaacaggagagcgcacgagggagcttccagggggaaacgcctg |
| | | gtatctttatagtcctgtcgggtttcgccacctctgacttgagcgtcgatttttgtgatgctcgtcaggggggcggagcct |
| | | atggaaaaacgccagcaacgcggcctttttacggttcctggccttttgctggccttttgctcacatgttctttcctgcgttat |
| | | cccctgattctgtggataaccgtattaccgcctttgagtgagctgataccgctcgcgcagccgaacgaccgagcgca |
| | | gcgagtcagtgagcgaggaagcggaagagcgcccaatacgcaaaccgcctctccccgcgcgttggccgattcatt |
| | | aatgcagctggcacgacaggtttcccgactggaaagcgggcagtgagcgcaacgcaattaatgtgagttagctcact |
| | | cattaggcaccccaggctttacactttatgcttccggctcgtatgttgtgtggaattgtgagcggataacaatttcacaca |
| | | ggaaacagctatgaccatgattacgcc |

TABLE 21

| | | | | | | | | | | Co- | | | | | | |
|---|---|---|---|---|---|---|---|---|---|---|---|---|---|---|---|---|
| SB | Descrip- | Back- | Seq | | | | | | | stim | CD3z | | | | Cleavage | TM |
| ID | tion | bone | Type | SS | scFV | Linker | scFV | Hinge | TM | ICD | ICD | E2A | SS | IL15 | Site | domain |
| SB06 | | | | GM- | hPY7 | (GGGG | hPY7 | CD8 | OX40 | OX40 | CD3z | E2A/ | IgE | IL15 | LR1 | B7-1 |
| 251 | | | | CSF- | VL | S)3 | VH | S2L | | | | T2A | | | split | |
| | | | | Ra | | (SEQ ID | | | | | | | | | N | |
| | | | | | | NO: | | | | | | | | | term | |
| | | | | | | 223) | | | | | | | | | linker | |
| | | | | | | | | | | | | | | | + Tace10 | |
| | GM-CSF- | | DNA | ATG | GACATCGTG | GGCGG | GAAGTGCAG | ACAA | GTG | GCTC | AGAGTGAAG | GGTAGCG | ATGG | AATTGGGTCAAC | TCTGGCG | CTGCTGC |
| | Ra(SS)- | | | CTG | ATGACACAG | CGGAG | CTGGTTGAA | CAAC | GCC | TGTA | TTCAGCAGA | GCCAGTG | ACTG | GTGATCAGCGAC | GCGGAGG | CTAGCTG |
| | aGPC3 | | | CTG | AGCCCCGAT | GATCT | TCAGGTGGC | CCCT | GCC | TCTG | AGCGCCGAC | TACCAAC | GACC | CTGAAGAAGATC | ATCTGGC | GGCCATC |
| | hPY7 vL | | | CTG | AGCCTGGCC | GGCGG | GGCCTGGTT | GCTC | ATTC | CTGC | GCACCCGCC | TACGCCC | TGGA | GAGGACCTGATC | GGAGGTG | ACACTGA |
| | - | | | GTC | GTGTCTCTGG | AGGTG | CAACCTGGC | CTAG | TCG | GGAG | TATAAGCAG | TGCTGAA | TCCT | CAGAGCATGCAC | GAAGCGG | TCTCCGT |
| | (GGGGS) | | | ACA | GAGAAAGAG | GAAGT | GGATCTCTG | ACCT | GAC | GGAC | GGACAGAAC | ACTGGCC | GTTT | ATCGACGCCACA | AGTTACA | GAACGGC |
| | 3(SEQ | | | TCT | CCACCATCA | GGCGG | AGACTGAGC | CCTA | TGG | CAAA | CAGCTGTAC | GGCGACG | CTGG | CTGTACACCGAG | CCCGAGC | ATCTTCG |
| | ID NO: | | | CTG | ACTGCAAGA | AGGCG | TGTGCCGCC | CACC | GAC | GACT | AACGAGCTG | TGGAATC | TGGC | AGCGACGTGCAC | CTATCTTC | TGATCTG |
| | 223)- | | | CTG | GCAGCCAGA | GATCT | AGCGGCTTC | AGCT | TTGT | GCCT | AACCTGGGG | TAATCCT | CGCT | CCTAGCTGTAAA | AGCCTGA | CTGCCTG |
| | aGPC3 | | | CTG | GCCTGCTGT | | ACCTTCAAC | CCTA | TCTG | CCTG | AGAAGAGAA | GGACCTG | GCCA | GTGACCGCCATG | TCGGAGG | ACCTACT |
| | hPY7- | | | TGC | ACTCCAGCA | | AAGAACGCC | CAAT | GGA | ATGC | GAGTACGAC | GATCTGG | CAA | AAGTGCTTTCTG | CGGTAGC | GCTTCGC |
| | CD8S2L | | | GAG | ACCAGAAGA | | ATGAACTGG | CGCC | CTGC | TCAC | GTGCTGGAC | CGAGGGA | GAGT | CTGGAACTGCAA | GGAGGCG | CCCTAGA |
| | (Hinge)- | | | CTG | ACTACCTGG | | GTCCGACAG | CTGC | TGG | AAGC | AAGCGGAGA | CGCGGGA | GCAC | GTGATCAGCCTG | GAGGAAG | TGCAGAG |
| | OX40 | | | CCC | CCTGGTATC | | GCCCCTGGC | AGCC | GAC | CTCC | GGCAGAGAT | GTCTACT | AGC | GAAAGCGGCGAC | TGGTGGC | AGCGGCG |
| | (TM)- | | | CAT | AGCAAAAGC | | AAAGGCCTT | TCTG | CTCT | AGGC | CCTGAGATG | GACGTGT | | GCCAGCATCCAC | GGATCTC | GAGAAAC |
| | OX40 | | | CCT | CCGGCCAGC | | GAATGGGTC | TCTCT | GGC | GGAG | GGCGGCAAG | GGAGACG | | GACACCGTGGAA | TGCAA | GAACGGC |
| | (ICD)- | | | GCC | CTCCTAAGCT | | GGACGGATC | GAGG | CATT | GCAG | CCCAGACGG | TGGAGGA | | AACCTGATCATC | | TGAGAAG |
| | CD3z | | | TTT | GCTGATCTAT | | CGGAACAAG | CCAG | CTGC | CTTC | AAGAATCCT | AAACCCT | | CTGGCCAACAAC | | AGAATCT |
| | (ICD)- | | | CTG | TGGGCCAGC | | ACCAACAAC | AAGC | T | AGAA | CAAGAGGGC | GGACCT | | AGCCTGAGCAGC | | GTGCGGC |
| | E2AT2A | | | CTG | TCCAGAGAA | | TACGCCACC | TTGT | | CCCC | CTGTATAAT | | | AACGGCAATGTG | | CCGTT |
| | -IgE(SS) | | | ATC | AGCGGCGTG | | TACTACGCC | AGAC | | TATC | GAGCTGCAG | | | ACCGAGTCCGGC | | |
| | -IL15- | | | CCT | CCCGATAGA | | GACAGCGTG | CAGC | | CAAG | AAAGACAAG | | | TGCAAAGAGTGC | | |
| | Tace10 | | | | TTTTCTGGCT | | AAGGCCAGA | TGCT | | AGGA | ATGGCCGAG | | | GAGGAACTGGAA | | |
| | (cleavage | | | | CTGGCAGCG | | TTCACCATCA | CGCC | | ACAG | GCCTACAGC | | | GAGAAGAATATC | | |
| | site)-B7- | | | | GCACCGACT | | GCCGGGACG | GGCG | | GCCG | GAGATCGGA | | | AAAGAGTTCCTG | | |
| | 1(TM) | | | | TCACCCTGA | | ACAGCAAGA | GAGC | | ACGC | ATGAAGGGC | | | CAGAGCTTCGTG | | |
| | | | | | CAATTTCTAG | | ACAGCCTGT | CGTG | | TCAC | GAGCGCAGA | | | CACATCGTGCAG | | |
| | | | | | CCTGCAAGC | | ACCTGCAGA | CATA | | AGCA | AGAGGCAAG | | | ATGTTCATCAAC | | |
| | | | | | CGAGGACGT | | TGAACTCCCT | CAAG | | CCCT | GGACACGAT | | | ACAAGC | | |
| | | | | | GGCCGTGTA | | GAAAACCGA | AGGA | | GGCC | GGACTGTAC | | | | | |
| | | | | | CTACTGCCA | | GGACACCGCC | CTGG | | AAGA | CAGGGCCTG | | | | | |
| | | | | | GCAGTACTA | | GTGTATTAT | ACTT | | TT | AGCACCGCC | | | | | |
| | | | | | CAACTACCC | | TGCGTGGCC | TGTG | | | ACCAAGGAT | | | | | |
| | | | | | TCTGACCTTC | | GGCAACAGC | AT | | | ACCTATGAT | | | | | |
| | | | | | GGCCAGGGC | | TTTGCCTACT | | | | GCCCTGCAC | | | | | |
| | | | | | ACCAAGCTG | | GGGGACAGG | | | | ATGCAGGCC | | | | | |
| | | | | | GAAATCAAA | | GAACCCTGG | | | | CTGCCTCCA | | | | | |
| | | | | | | | TCACCGTGTC | | | | AGA | | | | | |
| | | | | | | | TGCC | | | | | | | | | |
| | SEQ ID | | | 217 | 221 | 224 | 330 | 227 | 235 | 270 | 278 | 282 | 214 | 286 | 288 | 331 |
| | NO | | | | | | | | | | | | | | | |
| | | | AA | MLL | DIVMTQSPDS | GGGGS | EVQLVESGG | TTTP | VAAI | ALYL | RVKFSRSADA | GSGQCTN | MDW | NWVNVISDLKKIE | SGGGGSG | LLPSWAIT |
| | | | | LVT | LAVSLGERAT | GGGGS | GLVQPGGSLR | APRPPT | LGLG | LRRD | PAYKQGQNQ | YALLKLA | TWIL | DLIQSMHIDATLY | GGGSGVT | LISVNGIF |
| | | | | SLL | INCKSSQSLL | GGGGS | LSCAASGFT | PAPTI | LVLG | QRLPP | LYNELNLGR | GDVESNP | FLVA | TESDVHPSCKVTA | PEPIFSLI | VICCLTYC |
| | | | | LCE | YSSNQKNYL | | FNKNAMNWV | ALQP | LLGP | DAHK | REEYDVLDK | GPGSGEG | AATR | MKCFLLELQVISL | GGGSGGGG | FAPRCRER |
| | | | | LPH | AWYQQKPGQ | | RQAPGKGLE | LSLRP | LAIL | PPGG | RRGRDPEMG | RGSLLTCG | VHS | ESGDASIHDTVEN | SGGGSL | RRNERLR |
| | | | | PAF | PPKLLIYWAS | | WVGRIRNKT | EACR | | GSFRT | GKPRRKNPQ | DVEENPG | | LIILANNSLSSNGN | | RESVRPV |
| | | | | LLIP | SRESGVPDRF | | NNYATYYAD | PAAG | | PIQEE | EGLYNELQK | P | | VTESGCKECEELE | | |
| | | | | | SGSGSGTDFT | | SVKARFTISR | GAVH | | QADA | DKMAEAYSEI | | | EKNIKEFLQSFVHI | | |
| | | | | | LTISSLQAED | | DDSKNSLYLQ | TRGL | | HSTL | GMKGERRRG | | | VQMFINTS | | |
| | | | | | VAVYYCQQY | | MNSLKTEDTA | DFAC | | AKI | KGHDGLYQG | | | | | |
| | | | | | YNYPLTFGQG | | VYYCVAGN | | | | LSTATKDTYD | | | | | |
| | | | | | TKLEIK | | SFAYWGQGT | | | | ALHMQALPP | | | | | |
| | | | | | | | LVTVSA | | | | R | | | | | |
| | SEQ ID | | | 216 | 208 | 223 | 206 | 226 | 234 | 269 | 277 | 281 | 218 | 285 | 287 | 219 |
| | NO | | | | | | | | | | | | | | | |
| SB06 | | | | GM- | hPY7 | (GGGG | hPY7 | CD8FA | CD8FA | CD28 | CD3z | E2A/T2A | IgE | IL15 | LR1 | B7-1 |
| 252 | | | | CSF- | VH | S)3 | VL | | | | | | | | split | |
| | | | | Ra | | (SEQ ID | | | | | | | | | N | |
| | | | | | | NO: | | | | | | | | | term | |
| | | | | | | 223) | | | | | | | | | linker | |
| | | | | | | | | | | | | | | | + Tace10 | |
| | GM-CSF- | | DNA | ATG | GAAGTGCAG | GGCGG | GACATCGTG | GGCG | ATCT | CGGA | AGAGTGAAG | GGTAGCG | ATGG | AATTGGGTCAAC | TCTGGCG | ESKY |
| | Ra(SS)- | | | CTG | CTGGTGGAA | CGGAG | ATGACACAG | CCCT | ACA | GAGA | TTCAGCAGA | GCCAGTG | ACTG | GTGATCAGCGAC | GCGGAGG | GPPC |
| | aGPC3 | | | CTG | TCTGGCGGA | GAAGC | AGCCCCGAT | GAGC | TCTG | GACC | TCCGCCGAT | TACCAAC | GACC | CTGAAGAAGATC | ATCTGGC | PSCP |
| | hPY7 | | | CTG | GGACTGGTT | GGAGG | AGCCTGGCC | AACA | GGC | GCAA | GCTCCCGCC | TACGCCC | TGGA | GAGGACCTGATC | GGAGGTG | |
| | vH | | | GTC | CAACCTGGC | CGGAG | GTGTCTCTG | GCAT | CCCT | AGCA | TATCAGCAG | TGCTGAA | TCCT | CAGAGCATGCAC | GAAGCGG | |
| | (GGGGS) | | | ACA | GGCTCTCTG | GATCC | GAGGAAAGAG | CATG | CTG | GACT | GGACAGAAC | ACTGGCC | GTTT | ATCGACGCCACA | AGTTACA | |
| | 3(SEQ | | | TCT | AGACTGTCTT | GGTGG | CCACCATCA | TACT | GCT | GCTG | CAGCTGTAC | GGCGACG | CTGG | CTGTACACCGAG | CCCGAGC | |
| | ID NO: | | | CTG | GTGCCGCCA | TGGTG | ACTGCAAGA | TCAG | GGA | CACA | AACGAGCTG | TGGAATC | TGGC | AGCGACGTGCAC | CTATCTTC | |
| | 223)- | | | CTG | GCGGCTTCA | GATCT | GCAGCCAGA | CCAC | ACA | GCGA | AACCTGGGG | TAATCCT | CGCT | CCTAGCTGTAAA | AGCCTGA | |
| | aGPC3 | | | CTG | CCTTCAACA | | GCCTGCTGT | TTCG | TGTG | CTAC | AGAAGAGAA | GGACCTG | GCCA | GTGACCGCCATG | TCGGAGG | |
| | hPY7 | | | TGC | AGAACGCCA | | ACTCCAGCA | TGCC | GTGT | ATGA | GAGTACGAC | GATCTGG | CAAG | AAGTGCTTTCTG | CGGTAGC | |
| | vL | | | GAG | TGAACTGGG | | ACCAGAAGA | CGTG | CCTG | ACAT | GTGCTGGAC | CGAGGGA | AGT | CTGGAACTGCAA | GGAGGCG | |
| | -CD8FA | | | CTG | TCCGACAGG | | ACTACCTGG | TTTCT | CTGC | GACG | AAGCGGAGA | CGCGGGA | GCAC | GTGATCAGCCTG | GAGGAAG | |
| | (Hinge)- | | | CCC | CCCCTGGCA | | CCTGGTATC | GCCC | CCTA | GCCC | GGCAGAGAT | GTCTACT | AGC | GAAAGCGGCGAC | TGGTGGC | |
| | CD8FA | | | CAT | AAGGCCTTG | | AGCAAAAGC | GCCA | TGA | GGAC | CCTGAGATG | GACGTGT | | GCCAGCATCCAC | GGATCTC | |
| | (TM) | | | CCT | AATGGGTCG | | CCGGCCAGC | AGCC | GCCT | CTAC | GGCGGCAAG | GGAGACG | | GACACCGTGGAA | TGCAA | |
| | - | | | GCC | GACGGATCC | | CTCCTAAGCT | TACA | GGT | CAGA | CCCAGACGG | TGGAGGA | | AACCTGATCATC | | |
| | CD28 | | | TTT | GGAACAAGA | | GCTGATCTAT | ACAA | CATC | AAGC | AAGAATCCT | AAACCCT | | CTGGCCAACAAC | | |
| | (ICD) | | | CTG | CCAACAACT | | TGGGCCAGC | CCCC | ACC | ACTA | CAAGAGGGC | GGACCT | | AGCCTGAGCAGC | | |
| | - | | | CTG | ACGCCACCT | | TCCAGAGAA | TGCT | CTGT | CCAG | CTGTATAAT | | | AACGGCAATGTG | | |
| | CD3z | | | ATC | ACTACGCCG | | AGCGGCGTG | CCTA | ACT | CCTT | GAGCTGCAG | | | ACCGAGTCCGGC | | |
| | (ICD) | | | CCT | ACAGCGTGA | | CCCGATAGA | GACC | GCA | ACGC | AAAGACAAG | | | TGCAAAGAGTGC | | |
| | - | | | | AGGCCAGGT | | TTTTCTGGCT | TCCT | ACC | TCCT | ATGGCCGAG | | | GAGGAACTGGAA | | |
| | E2A | | | | TCACCATCTC | | CTGGCAGCG | ACAC | ACC | CCTA | GCCTACAGC | | | GAGAAGAATATC | | |
| | T2A | | | | CAGAGATGA | | GCACCGACT | CAGC | IGG | GAGA | GAGATCGGA | | | AAAGAGTTCCTG | | |
| | - | | | | CAGCAAGAA | | TCACCCTGA | ACAA | | CTTC | ATGAAGGGC | | | CAGAGCTTCGTG | | |
| | IgE | | | | CAGCCTGTA | | CAATTTCTAG | TCCT | | GCCG | GAGCGCAGA | | | CACATCGTGCAG | | |
| | (SS) | | | | CCTGCAGAT | | CCTGCAAGC | TCGC | | CCTA | AGAGGCAAG | | | ATGTTCATCAAC | | |
| | - | | | | GAACTCCCT | | CGAGGACGT | CAGC | | CCGG | GGACACGAT | | | ACAAGC | | |
| | IL15 | | | | GAAAACCGA | | GGCCGTGTA | CAGC | | TCC | GGACTGTAC | | | | | |
| | - | | | | GGACACCGC | | TTACTGCCA | CTCT | | | CAGGGCCTG | | | | | |
| | Tace10 | | | | CGTGTACTAT | | GCAGTACTA | GTCT | | | AGCACCGCC | | | | | |
| | (cleavage | | | | TGCGTGGCC | | CAACTACCC | CTGA | | | ACCAAGGAT | | | | | |
| | site) | | | | GGCAATAGC | | TCTGACCTTC | GGCC | | | ACCTATGAT | | | | | |
| | - | | | | TTTGCCTACT | | GGCCAGGGC | AGAA | | | GCCCTGCAC | | | | | |
| | B7- | | | | GGGGACAGG | | ACCAAGCTG | GCTT | | | ATGCAGGCC | | | | | |
| | 1 | | | | GCACCCTGG | | GAAATCAAA | GTAG | | | CTGCCTCCA | | | | | |
| | (TM) | | | | TTACAGTTTC | | | ACCT | | | AGA | | | | | |
| | | | | | TGCT | | | GCTG | | | | | | | | |
| | | | | | | | | CAGG | | | | | | | | |
| | | | | | | | | CGGA | | | | | | | | |
| | | | | | | | | GCCG | | | | | | | | |
| | | | | | | | | TGCA | | | | | | | | |
| | | | | | | | | TACA | | | | | | | | |
| | | | | | | | | AGAG | | | | | | | | |
| | | | | | | | | GACT | | | | | | | | |
| | | | | | | | | GGAT | | | | | | | | |
| | | | | | | | | TTCG | | | | | | | | |
| | | | | | | | | CCTG | | | | | | | | |
| | | | | | | | | CGAC | | | | | | | | |
| | SEQ | | | 217 | 222 | 332 | 333 | 229 | 243 | 268 | 280 | 282 | 214 | 286 | 288 | 331 |
| | ID | | | | | | | | | | | | | | | |
| | NO | | | | | | | | | | | | | | | |
| | | | AA | MLL | EVQLVESGG | GGGGS | DIVMTQSPDS | GALS | IYIW | RSKRS | RVKFSRS | GSGQCTN | MDW | NWVNVISDLKKIE | SGGGGSG | LLPSWAIT |
| | | | | LVT | GLVQPGGSLR | GGGGS | LAVSLGERAT | NSIM | APLA | RLLHS | ADAPAYQ | YALLKLA | TWIL | DLIQSMHIDATLY | GGGSGVT | LISVNGIF |
| | | | | SLL | LSCAASGFTF | GGGGS | INCKSSQSLL | YFSHF | GTC | DYMN | QGQNQLY | GDVESNP | FLVA | TESDVHPSCKVTA | PEPIFSLI | VICCLTYC |
| | | | | LCE | NKNAMNWV | | YSSNQKNYL | VPVFL | GVLL | MTPR | NELNLGR | GPGSGEG | AATR | MKCFLLELQVISL | GGGSGGG | FAPRCRER |
| | | | | LPH | RQAPGKGLE | | AWYQQKPGQ | PAKPT | LSLV | RPGPT | REEYDVL | RGSLLTC | VHS | LIILANNSLSSNG | GSGGGSL | RRNERLR |
| | | | | PAF | WVGRIRNKT | | PPKLLIYWAS | TTPAP | ITLY | RKHY | DKRRGRD | GDVEENP | | NVTESGCKECEEL | Q | RESVRPV |
| | | | | LLIP | NNYATYYAD | | SRESGVPDRF | RPPTP | CNH | QPYA | PEMGGKP | GP | | EESGDASIHDTVE | | |
| | | | | | SVKARFTISR | | SGSGSGTDFT | APTIA | R | PPRDF | RRKNPQE | | | NEKNIKEFLQSFV | | |
| | | | | | DDSKNSLYLQ | | LTISSLQAED | SQPLS | | AAYR | GLYNELQ | | | HIVQMFINTS | | |
| | | | | | MNSLKTEDT | | VAVYYCQQY | LRPEA | | S | KDKMAEA | | | | | |
| | | | | | AVYYCVAGN | | YNYPLTFGQG | CRPA | | | YSEIGMK | | | | | |
| | | | | | SFAYWGQGT | | TKLEIK | AGGA | | | GERRRGK | | | | | |
| | | | | | LVTVSA | | | VHTR | | | GHDGLYQ | | | | | |
| | | | | | | | | GLDF | | | GLSTATK | | | | | |
| | | | | | | | | ACD | | | DTYDALH | | | | | |
| | | | | | | | | | | | MQALPPR | | | | | |
| | SEQ ID | | | 216 | 206 | 223 | 208 | 228 | 242 | 267 | 279 | 281 | 218 | 285 | 287 | 219 |
| | NO | | | | | | | | | | | | | | | |
| SB06 | | | | GM- | hPY7 | (GGGG | hPY7 | CD8 | OX40 | OX40 | CD3z | E2A/T2A | IgE | IL15 | LR1 | B7-1 |
| 257 | | | | CSF- | VL | S)3 | VH | S2L | | | | | | | split | |
| | | | | Ra | | (SEQ ID | | | | | | | | | N | |
| | | | | | | NO: | | | | | | | | | term | |
| | | | | | | 223 | | | | | | | | | linker | |
| | | | | | | | | | | | | | | | + Tace10 | |
| | GM-CSF- | Sin | DNA | ATG | GACATCGTG | GGCGG | GAAGTGCAG | ACAA | GTG | GCTC | AGAGTGAAG | GGTAGCG | ATGG | AATTGGGTCAAC | TCTGGCG | CTGCTGC |
| | Ra(SS)- | Vec | | CTG | ATGACACAG | CGGAG | CTGGTTGAA | CAAC | GCC | TGTA | TTCAGCAGA | GCCAGTG | ACTG | GTGATCAGCGAC | GCGGAGG | CTAGCTG |
| | aGPC3 | | | CTG | AGCCCCGAT | GATCT | TCAGGTGGC | CCCT | GCC | TCTG | AGCGCCGAC | TACCAAC | GACC | CTGAAGAAGATC | ATCTGGC | GGCCATC |
| | hPY7 | | | CTG | AGCCTGGCC | GGCGG | GGCCTGGTT | GCTC | ATTC | CTGC | GCACCCGCC | TACGCCC | TGGA | GAGGACCTGATC | GGAGGTG | ACACTGA |
| | vL- | | | GTC | GTGTCTCTGG | AGGTG | CAACCTGGC | CTAG | TCG | GGAG | TATAAGCAG | TGCTGAA | TCCT | CAGAGCATGCAC | GAAGCGG | TCTCCGT |
| | (GGGGS) | | | ACA | GAGAAAGAG | GAAGT | GGATCTCTG | ACCT | GAC | GGAC | GGACAGAAC | ACTGGCC | GTTT | ATCGACGCCACA | AGTTACA | GAACGGC |
| | 3(SEQ | | | TCT | CCACCATCA | GGCGG | AGACTGAGC | CCTA | TGG | CAAA | CAGCTGTAC | GGCGACG | CTGG | CTGTACACCGAG | CCCGAGC | ATCTTCG |
| | ID NO: | | | CTG | ACTGCAAGA | AGGCG | TGTGCCGCC | CACC | GAC | GACT | AACGAGCTG | TGGAATC | TGGC | AGCGACGTGCAC | CTATCTTC | TGATCTG |
| | 223)- | | | CTG | ACCAGAAGA | GATCT | AGCGGCTTC | AGCT | TTGT | GCCT | AACCTGGGG | TAATCCT | CGCT | CCTAGCTGTAAA | AGCCTGAT | CTGCCTG |
| | aGPC3 | | | CTG | GCAGCCAGA | | ACCTTCAAC | CCTA | TCTG | CCTG | AGAAGAGAA | GGACCTG | GCCA | GTGACCGCCATG | CGGAGG | ACCTACT |
| | hPY7 | | | TGC | GCCTGCTGT | | AAGAACGCC | CAAT | GGA | ATGC | GAGTACGAC | GATCTGG | CAA | AAGTGCTTTCTG | CGGTAGC | GCTTCGC |
| | vH | | | GAG | ACTCCAGCA | | ATGAACTGG | CGCC | CTGC | TCAC | GTGCTGGAC | CGAGGGA | GAGT | CTGGAACTGCAA | GGAGGCG | CCCTAGA |
| | -CD8 | | | CTG | ACTACCTGG | | GTCCGACAG | CTGC | TGG | AAGC | AAGCGGAGA | CGCGGGA | GCAC | GTGATCAGCCTG | GAGGAAG | TGCAGAG |
| | S2L | | | CCC | CCTGGTATC | | GCCCCTGGC | AGCC | GAC | CTCC | GGCAGAGAT | GTCTACT | AGC | GAAAGCGGCGAC | TGGTGGC | AGCGGCG |
| | (Hinge)- | | | CAT | AGCAAAAGC | | AAAGGCCTT | TCTG | CTCT | AGGC | CCTGAGATG | GACGTGT | | GCCAGCATCCAC | GGATCTC | GAGAAAC |
| | OX40 | | | CCT | CCGGCCAGC | | GAATGGGTC | TCTCT | GGC | GGAG | GGCGGCAAG | GGAGACG | | GACACCGTGGAA | TGCAA | GAACGGC |
| | (TM)- | | | GCC | CTCCTAAGCT | | GGACGGATC | GAGG | CATT | GCAG | CCCAGACGG | TGGAGGA | | AACCTGATCATC | | TGAGAAG |
| | OX40 | | | TTT | GCTGATCTAT | | CGGAACAAG | CCAG | CTGC | CTTC | AAGAATCCT | AAACCCT | | CTGGCCAACAAC | | AGAATCT |
| | (ICD)- | | | CTG | TGGGCCAGC | | ACCAACAAC | AAGC | TG | AGAA | CAAGAGGGC | GGACCT | | AGCCTGAGCAGC | | GTGCGGC |
| | CD3z | | | CTG | TCCAGAGAA | | TACGCCACC | TTGT | | CCCC | CTGTATAAT | | | AACGGCAATGTG | | CCGTT |
| | (ICD)- | | | ATC | AGCGGCGTG | | TACTACGCC | AGAC | | TATC | GAGCTGCAG | | | ACCGAGTCCGGC | | |
| | E2A | | | CCT | CCCGATAGA | | GACAGCGTG | CAGC | | CAAG | AAAGACAAG | | | TGCAAAGAGTGC | | |
| | T2A | | | | TTTTCTGGCT | | AAGGCCAGA | TGCT | | AGGA | ATGGCCGAG | | | GAGGAACTGGAA | | |
| | - | | | | CTGGCAGCG | | TTCACCATCA | GGCG | | ACAG | GCCTACAGC | | | GAGAAGAATATC | | |
| | IgE | | | | GCACCGACT | | GCCGGGACG | GAGC | | GCCG | GAGATCGGA | | | AAAGAGTTCCTG | | |
| | (SS) | | | | TCACCCTGA | | ACAGCAAGA | CGTG | | ACGC | ATGAAGGGC | | | CAGAGCTTCGTG | | |
| | - | | | | CAATTTCTAG | | ACAGCCTGT | CATA | | TCAC | GAGCGCAGA | | | CACATCGTGCAG | | |
| | IL15 | | | | CCTGCAAGC | | ACCTGCAGA | CAAG | | AGCA | AGAGGCAAG | | | ATGTTCATCAAC | | |
| | - | | | | CGAGGACGT | | TGAACTOCCT | AGGA | | CCCT | GGACACGAT | | | ACAAGC | | |
| | Tace10 | | | | GGCCGTGTA | | GAAAACCGA | CTGG | | GGCC | GGACTGTAC | | | | | |
| | (cleavage | | | | CTACTGCCA | | GGACACCGC | ACTT | | AAGA | CAGGGCCTG | | | | | |
| | site) | | | | GCAGTACTA | | CGTGTATTAT | CGCC | | TT | AGCACCGCC | | | | | |
| | - | | | | CAACTACCC | | TGCGTGGCC | TGTG | | | ACCAAGGAT | | | | | |
| | B7- | | | | TCTGACCTTC | | GGCAACAGC | AT | | | ACCTATGAT | | | | | |
| | 1 | | | | GGCCAGGGC | | TTTGCCTACT | | | | GCCCTGCAC | | | | | |
| | (TM) | | | | ACCAAGCTG | | GGGGACAGG | | | | ATGCAGGCC | | | | | |
| | | | | | GAAATCAAA | | GAACCCTGG | | | | CTGCCTCCA | | | | | |
| | | | | | | | TCACCGTGTC | | | | AGA | | | | | |
| | | | | | | | TGCC | | | | | | | | | |
| | SEQ | | | 217 | 221 | 224 | 330 | 227 | 245 | 270 | 278 | 282 | 214 | 286 | 288 | 331 |
| | ID | | | | | | | | | | | | | | | |
| | NO | | | | | | | | | | | | | | | |
| | | | AA | MLL | DIVMTQSPDS | GGGGS | EVQLVESGG | TTTPA | VAAI | ALYL | RVKFSRSADA | GSGQCTN | MDW | NWVNVISDLKKIE | SGGGGSG | LLPSWAIT |
| | | | | LVT | LAVSLGERAT | GGGGS | GLVQPGGSLR | PRPPT | LGLG | LRRD | PAYKQGQNQ | YALLKLA | TWIL | DLIQSMHIDATLY | GGGSGVT | LISVNGIF |
| | | | | SLL | INCKSSQSLL | GGGGS | LSCAASGFTF | PAPTI | LVLG | QRLPP | LYNELNLGR | GDVESNP | FLVA | TESDVHPSCKVTA | PEPIFSLI | VICCLTYC |
| | | | | LCE | YSSNQKNYL | | NKNAMNWV | ALQP | LLGP | DAHK | REEYDVLDK | GPGSGEG | AATR | MKCFLLELQVISL | GGGSGGG | FAPRCRER |
| | | | | LPH | AWYQQKPGQ | | RQAPGKGLE | LSLRP | LAIL | PPGG | RRGRDPEMG | RGSLLTCG | VHS | ESGDASIHDTVEN | GSGGGSL | RRNERLR |
| | | | | PAF | PPKLLIYWAS | | WVGRIRNKT | EACR | | GSFRT | GKPRRKNPQ | DVEENPG | | LIILANNSLSSNG | Q | RESVRPV |
| | | | | LLIP | SRESGVPDRF | | NNYATYYAD | PAAG | | PIQEE | EGLYNELQK | P | | NVTESGCKECEEL | | |
| | | | | | SGSGSGTDFT | | SVKARFTIS | GAVH | | QADA | DKMAEAYSEI | | | EEKNIKEFLQSFV | | |
| | | | | | LTISSLQAED | | RDDSKNSL | TRGL | | HSTL | GMKGERRRG | | | HIVQMFINTS | | |
| | | | | | VAVYYCQQY | | YLQMNSLKT | DFAC | | AKI | KGHDGLYQG | | | | | |
| | | | | | YNYPLTFGQG | | EDTAVYYCV | D | | | LSTATKDTYD | | | | | |
| | | | | | TKLEIK | | AGNSFAYWG | | | | ALHMQALPP | | | | | |
| | | | | | | | QGTLVTVSA | | | | R | | | | | |
| | SEQ | | | 216 | 208 | 223 | 206 | 226 | 244 | 269 | 277 | 281 | 218 | 285 | 287 | 219 |
| | ID | | | | | | | | | | | | | | | |
| | NO | | | | | | | | | | | | | | | |
| SB06 | | | | GM- | hPY7 | (GGGG | hPY7 | CD8FA | CD8FA | CD28 | CD3z | E2A/ | IgE | IL15 | LR1 | B7-1 |
| 258 | | | | CSF- | VH | S)3 | VL | | | | | T2A | | | split | |
| | | | | Ra | | (SEQ | | | | | | | | | N | |
| | | | | | | ID | | | | | | | | | term | |
| | | | | | | NO: | | | | | | | | | linker | |
| | | | | | | 223) | | | | | | | | | + Tace10 | |
| | GM-CSF- | Sin | DNA | ATG | GAAGTGCAG | GGCGG | GACATCGTG | GGCGC | ATCT | CGGA | AGAGTGAAG | GGTAGCG | ATGG | AATTGGGTCAAC | TCTGGCG | CTGCTGC |
| | Ra(SS)- | Vec | | CTG | CTGGTTGAA | CGGAG | ATGACACA | CCTGA | ACA | GCAA | TTCAGCAGA | GCCAGTG | ACTG | GTGATCAGCGAC | GCGGAGG | CTAGCTG |
| | aGPC3 | | | CTG | TCAGGTGGC | GATCT | GAGCCCCG | GCAAC | TCTG | GAGA | TCCGCCGAT | TACCAAC | GACC | CTGAAGAAGATC | ATCTGGC | GGCCATC |
| | hPY7 | | | CTG | GGCCTGGTT | GGCGG | ATAGCCTGG | AGCAT | GGC | AGCA | GCTCCCGCC | TACGCCC | TGGA | GAGGACCTGATC | GGAGGTG | ACACTGA |
| | vH | | | GTC | CAACCTGGC | AGGTG | CCGTGTCTC | CATGT | CCCT | GACT | TATCAGCAG | TGCTGAA | TCCT | CAGAGCATGCAC | GAAGCGG | TCTCCGT |
| | (GGGGS) | | | ACA | GGATCTCTG | GAAGT | TGGGAGAA | CCAGA | CTG | GCTG | GGACAGAAC | ACTGGCC | GTTT | ATCGACGCCACA | AGTTACA | TGATCTG |
| | 3(SEQ | | | TCT | AGACTGAGC | GGCGG | AGAGCCAC | GTAGA | GCT | CACA | CAGCTGTAC | GGCGACG | CTGG | CTGTACACCGAG | CCCGAGC | GAACGGC |
| | ID NO: | | | CTG | TGTGCCGCC | AGGCG | CATCAACTG | ACTTC | GGA | GCGA | AACGAGCTG | TGGAATC | TGGC | AGCGACGTGCAC | CTATCTT | ATCTTCG |
| | 223)- | | | CTG | AGCGGCTTC | GATCT | CAAGAGCA | AGCCA | ACA | CTAC | AACCTGGGG | TAATCCT | CGCT | CCTAGCTGTAAA | CAGCCTG | CTGCCTG |
| | aGPC3 | | | CTG | ACCTTCAAC | | GCCAGAGC | CTTCG | TGTG | ATGA | AGAAGAGAA | GGACCTG | GCCA | GTGACCGCCATG | ATCGGAGG | ACCTACT |
| | hPY7 | | | TGC | AAGAACGCC | | CTGCTGTAC | TGCCC | GTGT | ACAT | GAGTACGAC | GATCTGG | CAA | AAGTGCTTTCTG | CGGTAGC | GCTTCGC |
| | vL | | | GAG | ATGAACTGG | | TCCAGCAAC | GTGTT | CCTG | GACC | GTGCTGGAC | CGAGGGA | GAGT | CTGGAACTGCAA | GGAGGCG | CCCTAGA |
| | -CD8FA | | | CTG | GTCCGACAG | | CAGAAGAA | TCTGC | CTGC | CCTA | AAGCGGAGA | CGCGGGA | GCAC | GTGATCAGCCTG | GAGGAAG | TGCAGAG |
| | (Hinge)- | | | CCC | GCCCCTGGC | | CTACCTGGC | CCGCC | TGA | GACG | GGCAGAGAT | GTCTACT | AGC | GAAAGCGGCGAC | TGGTGGC | AGCGGCG |
| | CD8 | | | CAT | AAAGGCCTT | | CTGGTATCA | AAGCC | GCCT | GCCC | CCTGAGATG | GACGTGT | | GCCAGCATCCAC | GGATCTC | GAGAAAC |
| | (TM)- | | | CCT | GAATGGGTC | | GCAAAAGC | TACAA | GGT | GGAC | GGCGGCAAG | GGAGACG | | GACACCGTGGAA | TGCAA | GAACGGC |
| | CD28 | | | GCC | GGACGGATC | | CCGGCCAG | CAACC | CATC | CTAC | CCCAGACGG | TGGAGGA | | AACCTGATCATC | | TGAGAAG |
| | (ICD)- | | | TTT | CGGAACAAG | | CCTCCTAAG | CCTGC | ACC | CAGA | AAGAATCCT | AAACCCT | | CTGGCCAACAAC | | AGAATCT |
| | CD3z | | | CTG | ACCAACAAC | | CTGCTGATC | TCCTA | CTGT | AAGC | CAAGAGGGC | GGACCT | | AGCCTGAGCAGC | | GTGCGGC |
| | (ICD)- | | | CTG | TACGCCACC | | TATTGGGCC | GACCT | ACT | ACTA | CTGTATAAT | | | AACGGCAATGTG | | CCGTT |
| | E2AT2A | | | ATC | TACTACGCC | | AGCTCCAG | CCTAC | GCA | CCAG | GAGCTGCAG | | | ACCGAGTCCGGC | | |
| | -IgE(SS) | | | CCT | GACAGCGTG | | AGAAAGCG | ACCAG | ACC | CCTT | AAAGACAAG | | | TGCAAAGAGTGC | | |
| | -IL15- | | | | AAGGCCAGA | | GCGTGCCCG | CTCCT | ACC | ACGC | ATGGCCGAG | | | GAGGAACTGGAA | | |
| | Tace10 | | | | TTCACCATCA | | ATAGATTTT | ACAAT | GG | TCCT | GCCTACAGC | | | GAGAAGAATATC | | |
| | (cleavage | | | | GCCGGGACG | | CTGGCTCTG | CGCCA | | CCTA | GAGATCGGA | | | AAAGAGTTCCTG | | |
| | site)-B7- | | | | ACAGCAAGA | | GCAGCGGC | GCCAG | | GAGA | ATGAAGGGC | | | CAGAGCTTCGTG | | |
| | 1(TM) | | | | ACAGCCTGT | | ACCGACTTC | CCTCT | | CTTC | GAGCGCAGA | | | CACATCGTGCAG | | |
| | | | | | ACCTGCAGA | | ACCCTGACA | GTCTC | | GCCG | AGAGGCAAG | | | ATGTTCATCAAC | | |
| | | | | | TGAACTCCCT | | ATTTCTAGC | TGAGG | | CCTA | GGACACGAT | | | ACAAGC | | |
| | | | | | GAAAACCGA | | CTGCAAGCC | AGCTT | | CCGG | GGACTGTAC | | | | | |
| | | | | | GGACACCGC | | GAGGACGTG | TGCAG | | TCC | CAGGGCCTG | | | | | |
| | | | | | CGTGTATTAT | | GCCGTGTA | GCGGA | | | AGCACCGCC | | | | | |
| | | | | | TGCGTGGCC | | CTACTGCCA | GCCGT | | | ACCAAGGAT | | | | | |
| | | | | | GGCAACAGC | | GCAGTACTA | TCGCC | | | ACCTATGAT | | | | | |
| | | | | | TTTGCCTACT | | CAACTACCC | CCTGC | | | GCCCTGCAC | | | | | |
| | | | | | GGGGACAGG | | TCTGACCTT | GCATA | | | ATGCAGGCC | | | | | |
| | | | | | GAACCCTGG | | CGGCCAGG | CAAGA | | | CTGCCTCCA | | | | | |
| | | | | | TCACCGTGTC | | GCACCAAG | GGACT | | | AGA | | | | | |
| | | | | | TGCC | | CTGGAAATC | GGATT | | | | | | | | |
| | | | | | | | AAA | TGCGA | | | | | | | | |
| | | | | | | | | C | | | | | | | | |
| | SEQ ID | | | 217 | 330 | 224 | 221 | 229 | 243 | 268 | 280 | 282 | 214 | 286 | 288 | 331 |
| | NO | | | | | | | | | | | | | | | |
| | | | AA | MLL | EVQLVESGG | GGGGS | DIVMTQSPD | GALSN | IYIW | RSKRS | RVKFSRSA | GSGQCTN | MDW | NWVNVISDLKKIE | SGGGGSG | LLPSWAIT |
| | | | | LVT | GLVQPGGSLR | GGGGS | SLAVSLGER | SIMYFS | APLA | RLLHS | DAPAYQQG | YALLKLA | TWIL | DLIQSMHIDATLY | GGGSGVT | LISVNGIF |
| | | | | SLL | LSCAASGFTF | GGGGS | ATINCKSSQS | HFVPV | GTC | DYMN | QNQLYNEL | GDVESNP | FLVA | TESDVHPSCKVTA | PEPIFSLI | VICCLTYC |
| | | | | LCE | NKNAMNWV | | LLYSSNQKN | FLPAK | GVLL | MTPR | NLGRREEY | GPGSGEG | AATR | MKCFLLELQVISL | GGGSGGG | FAPRCRER |
| | | | | LPH | RQAPGKGLE | | YLAWYQQKP | PTTTPA | LSLV | RPGPT | DVLDKRRG | RGSLLTCG | VHS | ESGDASIHDTVEN | GSGGGSL | RRNERLR |
| | | | | PAF | WVGRIRNKT | | GQPPKLLIY | PRPPTP | ITLY | RKHY | RDPEMGGK | DVEENPG | | LIILANNSLSSNG | Q | RESVRPV |
| | | | | LLIP | NNYATYYAD | | WASSRESGV | APTIAS | CNH | QPYA | PRRKNPQE | P | | NVTESGCKECEEL | | |
| | | | | | SVKARFTISR | | PDRFSGSGS | QPLSL | R | PPRDF | GLYNELQK | | | EEKNIKEFLQSFV | | |
| | | | | | DDSKNSLYLQ | | GTDFTLTISS | RPEAC | | AAYR | DKMAEAYS | | | HIVQMFINTS | | |
| | | | | | MNSLKTEDT | | LQAEDVAV | RPAAG | | S | EIGMKGER | | | | | |
| | | | | | AVYYCVAGN | | YYCQQYYN | GAVHT | | | RRGKGHDG | | | | | |
| | | | | | SFAYWGQGT | | YPLTFGQGT | RGLDF | | | LYQGLSTA | | | | | |
| | | | | | LVTVSA | | KLEIK | ACD | | | TKDTYDAL | | | | | |
| | | | | | | | | | | | HMQALPPR | | | | | |
| | SEQ | | | 216 | 206 | 223 | 208 | 228 | 242 | 267 | 279 | 281 | 218 | 285 | 287 | 219 |
| | ID | | | | | | | | | | | | | | | |
| | NO | | | | | | | | | | | | | | | |
| SB06 | | | | GM- | hPY7 | (GGGG | hPY7 | CD8FA | CD8 | CD28 | CD3z | E2A/ | IgE | IL15 | LR1 | B7-1 |
| 298 | | | | CSF- | VH | S)3 | VL | | FA | | | T2A | | | split | |
| | | | | Ra | | (SEQ | | | | | | | | | term | |
| | | | | | | ID | | | | | | | | | linker | |
| | | | | | | NO: | | | | | | | | | + Tace10 | |
| | | | | | | 223) | | | | | | | | | | |
| | GM-CSF- | Sin | DNA | ATG | GAAGTGCAG | GGCGG | GACATCGTG | GGCGC | GGC | CGGA | AGAGTGAAG | CAGTGTA | ATGG | AATTGGGTCAAC | ATCTGGC | ACACTGA |
| | Ra | Vec | | CTG | CTGGTGGAA | GAAGC | ATGACACA | CCTGA | ATCT | GCAA | TTCAGCAGG | CCAACTA | ACTG | GTGATCAGCGAC | GGAGGTG | CTGCTGC |
| | (SS)- | | | CTG | TCTGGCGGA | CGGAG | GAGCCCCG | GCAAC | CCCT | GAGA | AGCGCAGAC | CGCCCTG | GACC | CTGAAGAAGATC | TCTGGCG | CTAGCTG |
| | aGPC3 | | | CTG | GGACTGGTT | GGAGG | ATAGCCTGG | AGCAT | ACA | AGCA | GCCCCCGCG | CTGAAAC | TGGA | GAGGACCTGATC | GCGGAGG | GGCCATC |
| | hPY7 vH | | | GTC | CAACCTGGC | CGGAG | CCGTGTCTC | CATGT | TCTG | GACT | TACAAGCAG | TGGCCGG | TCCT | CAGAGCATGCAC | GAAGCGG | TCTCCGT |
| | (GGGGS) | | | ACA | GGCTCTCTG | GATCC | TGGGAGAA | ACTTC | CTG | GCTG | GGCCAGAAC | CGACGTG | GTTT | ATCGACGCCACA | AGTTACA | GAACGGC |
| | 3(SEQ | | | TCT | AGACTGTCTT | GGTGG | AGAGCCAC | AGCCA | GCT | CACA | CAGCTCTAT | GAATCTA | CTGG | CTGTACACCGAG | CCCGAGC | ATCTTCG |
| | ID NO: | | | CTG | GTGCCGCCA | TGGTG | CATCAACTG | CTTCG | GGA | GCGA | AACGAGCTC | ATCCTGG | TGGC | AGCGACGTGCAC | CTATCTTC | TGATCTG |
| | (223)- | | | CTG | GCGGCTTCA | GATCT | CAAGAGCA | TGCCC | ACA | CTAC | AATCTAGGA | ACCTGGA | CGCT | CCTAGCTCTAAA | AGCCTGA | CTGCCTC |
| | aGPC3 | | | CTG | CCTICAACA | | GCCAGAGC | GTGTT | TGTG | ATGA | CGAAGAGAG | TCTGGCG | GCCA | GTGACCGCCATG | TCGGAGG | ACCTACT |
| | hPY7 vL | | | TGC | AGAACGCCA | | CTGCTGTAC | TCTGC | GTGT | ACAT | GAGTACGAT | AGGGACG | CAA | AAGTGCTTTCTG | CGGTAGC | GCTTCGC |
| | -CD8FA | | | GAG | TGAACTGGG | | TCCAGCAAC | CCGCC | CCTG | GACC | GTTTTGGAC | CGGGAGT | GAGT | CTGGAACTGCAA | GGAGGCG | CCCTAGA |
| | (Hinge)- | | | CTG | TCCGACAGG | | CAGAAGAA | AAGCC | CTGC | CCTA | AAGAGACGT | CTACTGA | GCAC | GTGATCAGCCTG | GAGGAAG | TGCAGAG |
| | CD8 | | | CCC | CCCCTGGCA | | CTACCTGGC | TACAA | TGA | GACG | GGCCGGGAC | CGTGTGG | AGC | GAAAGCGGCGAC | TGGTGGC | JAGCGGCG |
| | (TM)- | | | CAT | AAGGCCTTG | | CTGGTATCA | CAACC | GCCT | GCCC | CCTGAGATG | AGACGTG | | GCCAGCATCCAC | GGATCTC | GAGAAAC |
| | CD28 | | | CCT | AATGGGTCG | | GCAAAAGC | CCTGC | GGT | GGAC | GGGGGAAAG | GAGGAAA | | GACACCGTGGAA | TGCAA | GAACGGC |
| | (ICD)- | | | GCC | GACGGATCC | | CCGGCCAG | TCCTA | CATC | CTAC | CCGAGAAGG | ACCCTGG | | AACCTGATCATC | | TGAGAAG |
| | CD3z mut | | | TTT | GGAACAAGA | | CCTCCTAAG | GACCT | ACC | CAGA | AAGAACCCT | ACCT | | CTGGCCAACAAC | | AGAATCT |
| | (ICD)- | | | CTG | CCAACAACT | | CTGCTGATC | CCTAC | CTGT | AAGC | CAGGAAGGC | | | AGCCTGAGCAGC | | GTGCGGC |
| | E2A T2A | | | CTG | ACGCCACCT | | TATTGGGCC | ACCAG | ACT | ACTA | CTGTACAAT | | | AACGGCAATGTG | | CCGTT |
| | -IgE (SS) | | | ATC | ACTACGCCG | | AGCTCCAG | CTCCT | GCA | CCAG | GAACTGCAG | | | ACCGAGTCCGGC | | |
| | -IL15- | | | CCT | ACAGCGTGA | | AGAAAGCG | ACAAT | ACC | CCTT | ANAGATAAG | | | TGCAAAGAGTGC | | |
| | Tace10 | | | | AGGCCAGGT | | GCGTGCCCG | CGCCA | ACC | ACGC | ATGGCGGAG | | | GAGGAACTGGAA | | |
| | (cleavage | | | | TCACCATCTC | | ATAGATTTT | GCCAG | GG | TCCT | GCCTACAGT | | | GAGAAGAATATC | | |
| | site)- | | | | CAGAGATGA | | CTGGCTCTG | CCTCT | | CCTA | GAGATTGGG | | | AAAGAGTTCCTG | | |
| | B7- | | | | CAGCAAGAA | | GCAGCGGC | GTCTC | | GAGA | ATGAAAGGC | | | CAGAGCTTCGTG | | |
| | 1 (TM) | | | | CAGCCTGTA | | ACCGACTTC | TGAGG | | CTTC | GAGCGCCGG | | | CACATCGTGCAG | | |
| | | | | | CCTGCAGAT | | ACCCTGACA | CCAGA | | GCCG | AGGGGCAAG | | | ATGTTCATCAAC | | |
| | | | | | GAACTCCCT | | ATTTCTAGC | AGCTT | | CCTA | GGGCACGAT | | | ACAAGC | | |
| | | | | | GAAAACCGA | | CTGCAAGCC | GTAGA | | CCGG | GGCCTTTAC | | | | | |
| | | | | | GGACACCGC | | GAGGACGT | CCTGC | | TCC | CAGGGTCTC | | | | | |
| | | | | | CGTGTACTAT | | GGCCGTGTA | TGCAG | | | AGTACAGCC | | | | | |
| | | | | | TGCGTGGCC | | TTACTGCCA | GCGGA | | | ACCAAGGAC | | | | | |
| | | | | | GGCAATAGC | | GCAGTACTA | GCCGT | | | ACCTACGAC | | | | | |
| | | | | | TTTGCCTACT | | CAACTACCC | GCATA | | | GCCCTTCAC | | | | | |
| | | | | | GGGGACAGG | | TCTGACCTT | CAAGA | | | ATGCAGGCC | | | | | |
| | | | | | GCACCCTGG | | CGGCCAGG | GGACT | | | CTGCCCCCT | | | | | |
| | | | | | TTACAGTTTC | | GCACCAAG | GGATT | | | CGC | | | | | |
| | | | | | TCCT | | CTGGAAATC | TCGCC | | | | | | | | |
| | | | | | | | AAA | TGCGA | | | | | | | | |
| | | | | | | | | C | | | | | | | | |
| | SEQ ID | | 217 | 222 | 332 | 333 | 229 | 243 | 268 | 334 | 284 | 214 | 286 | 288 | 331 | |
| | NO | | | | | | | | | | | | | | | |
| | | | AA | MLL | EVQLVESGG | GGGGS | DIVMTQSP | GALSN | IYIW | RSKRS | RVKFSRSADA | QCTNYAL | MDW | NWVNVISDLKKIE | SGGGGSG | LLPSWAIT |
| | | | | LVT | GLVQPGGSLR | GGGGS | DSLAVSLG | HFVPV | GTC | DYMN | PAYKQGQNQ | LKLAGDV | TWIL | DLIQSMHIDATLY | GGGSGVT | LISVNGIF |
| | | | | SLL | LSCAASGFTF | GGGGS | ERATINCK | SIMYFS | APLA | RLLHS | LYNELNLGR | ESNPGPGS | FLVA | TESDVHPSCKVTA | PEPIFSLI | VICCLTYC |
| | | | | LCE | NKNAMNWV | | SSQS LLY | FLPAK | GVLL | DYMN | REEYDVLDK | GEGRGSL | AATR | MKCFLLELQVISL | GGGSGGG | FAPRCRER |
| | | | | LPH | RQAPGKGLE | | SSNQKNYL | PTTTPA | LSLV | MTPR | RRGRDPEMG | LTCGDVE | VHS | ESGDASIHDTVEN | GSGGGSL | RRNERLR |
| | | | | PAF | WVGRIRNKT | | AWYQQKPG | PRPPTP | ITLY | RPGPT | GKPRRKNPQ | ENPGP | | LIILANNSLSSNG | Q | RESVRPV |
| | | | | LLIP | NNYATYYAD | | QPPKLLIY | APTIAS | CNH | RKHY | EGLYNELQK | | | NVTESGCKECEEL | | |
| | | | | | SVKARFTISR | | WASSRESG | QPLSL | R | QPYA | DKMAEAYSEI | | | EEKNIKEFLQSFV | | |
| | | | | | DDSKNSLYLQ | | VPDRFSGS | RPEAC | | PPRDF | GMKGERRRG | | | HIVQMFINTS | | |
| | | | | | MNSLKTEDT | | GSGTDFTL | RPAAG | | AAYR | KGHDGLYQG | | | | | |
| | | | | | AVYYCVAGN | | TISSLQAE | GAVHT | | S | LSTATKDTYD | | | | | |
| | | | | | SFAYWGQGT | | DVAVYYCQ | RGLDF | | | ALHMQALPP | | | | | |
| | | | | | LVTVSA | | QYYNYPLT | ACD | | | R | | | | | |
| | | | | | | | FGQGTKLE | | | | | | | | | |
| | | | | | | | IK | | | | | | | | | |
| | SEQ | | | 216 | 206 | 223 | 208 | 228 | 242 | 267 | 277 | 283 | 218 | 285 | 287 | 219 |
| | ID | | | | | | | | | | | | | | | |
| | NO | | | | | | | | | | | | | | | |
| SB06 | | | | IgE | IL15 | LR1 | B7-1 | E2A/T2 | GM- | hPY7 | (GGGGS) | hPY7 | CD8S2L | OX40 | OX40 | CD3z |
| 254 | | | | | | splitN | | A | CSF- | VL | 3(SEQ | VH | | | | |
| | | | | | | term | | | Ra | | ID NO: | | | | | |
| | | | | | | linker | | | | | 223) | | | | | |
| | | | | | | Tace10 | | | | | | | | | | |
| | IgE(SS)- | | DNA | ATG | AATTGGGTC | TCTGG | CTGCTG | CAGTG | ATG | GACATCGTG | GGCGG | GAAGTGCAG | ACAACA | GTG | GCTCTGTATCT | AGAGTGAAG |
| | IL15 | | | GAC | AACGTGATC | CGGCG | CCTAGC | TACCA | CTGC | ATGACACAG | CGGAG | CTGGTTGAA | ACCCCT | GCC | GCTGCGGAGG | TTCAGCAGA |
| | Tace10 | | | TGG | AGCGACCTG | GAGGA | TGGGCC | ACTAC | TGCT | AGCCCCGAT | GATCTG | TCAGGTGGC | GCTCCTA | GCC | GACCAAAGACT | AGCGCCGAC |
| | (cleavage | | | ACC | AAGAAGATC | TCTGG | ATCACA | GCCCT | GGT | AGCCTGGCC | GCGGA | GGCCTGGTT | GACCTC | ATTC | GCCTCCTGATG | GCACCCGCC |
| | site)-B7- | | | TGG | GAGGACCTG | CGGAG | CTGATC | GCTGA | CAC | GTGTCTCTGG | GGTGG | CAACCTGGC | CTACAC | TCG | CTCACAAGCCT | TATAAGCAG |
| | 1(TM)- | | | ATC | ATCCAGAGC | GTGGA | TCCGTG | AACTG | ATCT | GAGAAAGAG | AAGTG | GGATCTCTG | CAGCTC | GAC | CCAGGCGGAG | GGACAGAAC |
| | E2AT2A | | | CTG | ATGCACATC | AGCGG | AACGG | GCCGG | CTGC | CCACCATCA | GCGGA | AGACTGAGC | CTACAA | TGG | GCAGCTTCAGA | CAGCTGTAC |
| | -GM- | | | TTT | GACGCCACA | AGTTA | CATCTT | CGACG | TGCT | ACTGCAAGA | GGCGG | TGTGCCGCC | TCGCCCT | GAC | ACCCCTATCCA | AACGAGCTG |
| | CSF-Ra | | | CTG | CTGTACACC | CACCC | CGTGAT | TGGAA | GTG | GCAGCCAGA | ATCT | AGCGGCTTC | GCAGCC | TTGT | AGAGGAACAG | AACCTGGGG |
| | (SS)- | | | GTG | GAGAGCGAC | GAGCC | CTGCTG | TCTAA | CGA | GCCTGCTGT | | ACCTTCAAC | TCTGTCT | TCTG | GCCGACGCTCA | AGAAGAGAA |
| | aGPC3 | | | GCC | GTGCACCCT | TATCT | CCTGAC | TCCTG | GCT | ACTCCAGCA | | AAGAACGCC | CTGAGG | GGA | CAGCACCCTGG | GAGTACGAC |
| | hPY7 vL | | | GCT | AGCTGTAAA | TCAGC | CTACTG | GACCT | GCC | ACCAGAAGA | | ATGAACTGG | CCAGAA | CTGC | CCAAGATT | GTGCTGGAC |
| | (GGGGS) | | | GCC | GTGACCGCC | CTGAT | CTTCGC | GGATC | CCAT | ACTACCTGG | | GTCCGACAG | GCTTGTA | TGG | | AAGCGGAGA |
| | 3(SEQ | | | ACA | ATGAAGTGC | CGGAG | CCCTAG | TGGCG | CCTG | CCTGGTATC | | GCCCCTGGC | GACCAG | GAC | | GGCAGAGAT |
| | ID NO: | | | AGA | TTTCTGCTGG | GCGGT | ATGCAG | AGGGA | CCTT | AGCAAAAGC | | AAAGGCCTT | CTGCTG | CTCT | | CCTGAGATG |
| | 223)- | | | GTG | AACTGCAAG | AGCGG | AGAGC | CGCGG | TCTG | CCGGCCAGC | | GAATGGGTC | GCGGAG | GGC | | GGCGGCAAG |
| | aGPC3 | | | CAC | TGATCAGCC | AGGCG | GGCGG | GAGTC | CTG | CTCCTAAGCT | | GGACGGATC | CCGTGC | CATT | | CCCAGACGG |
| | hPY7 | | | AGC | TGGAAAGCG | GAGGA | AGAAA | TACTG | ATCC | GCTGATCTAT | | CGGAACAAG | ATACAA | CTGC | | AAGAATCCT |
| | vH | | | | GCGACGCCA | AGTGG | CGAAC | ACGTG | CT | TGGGCCAGC | | ACCAACAAC | GAGGAC | TG | | CAAGAGGGC |
| | -CD8 | | | | GCATCCACG | TGGCG | GGCTGA | TGGAG | | TCCAGAGAA | | TACGCCACC | TGGACTT | | | CTGTATAAT |
| | S2L | | | | ACACCGTGG | GATCT | GAAGA | ACGTG | | AGCGGCGTG | | TACTACGCC | CGCCTGT | | | GAGCTGCAG |
| | (Hinge)- | | | | AAAACCTGA | CTGCA | GAATCT | GAGGA | | CCCGATAGA | | GACAGCGTG | GAT | | | AAAGACAAG |
| | OX40 | | | | TCATCCTGGC | A | GTGCGG | AAACC | | TTTTCTGGCT | | AAGGCCAGA | | | | ATGGCCGAG |
| | (TM)- | | | | CAACAACAG | | CCCGTT | CTGGA | | CTGGCAGCG | | TTCACCATCA | | | | GCCTACAGC |
| | OX40 | | | | CCTGAGCAG | | | CCT | | GCACCGACT | | GCCGGGACG | | | | GAGATCGGA |
| | (ICD)- | | | | CAACGGCAA | | | | | TCACCCTGA | | ACAGCAAGA | | | | ATGAAGGGC |
| | CD3z | | | | TGTGACCGA | | | | | CAATTTCTAG | | ACAGCCTGT | | | | GAGCGCAGA |
| | (ICD) | | | | GTCCGGCTG | | | | | CCTGCAAGC | | ACCTGCAGA | | | | AGAGGCAAG |
| | | | | | CAAAGAGTG | | | | | CGAGGACGT | | TGAACTCCCT | | | | GGACACGAT |
| | | | | | CGAGGAACT | | | | | GGCCGTGTA | | GAAAACCGA | | | | GGACTGTAC |
| | | | | | GGAAGAGAA | | | | | CTACTGCCA | | GGACACCGC | | | | CAGGGCCTG |
| | | | | | GAATATCAA | | | | | GCAGTACTA | | CGTGTATTAT | | | | AGCACCGCC |
| | | | | | AGAGTTCCT | | | | | CAACTACCC | | TGCGTGGCC | | | | ACCAAGGAT |
| | | | | | GCAGAGCTT | | | | | TCTGACCTTC | | GGCAACAGC | | | | ACCTATGAT |
| | | | | | CGTGCACAT | | | | | GGCCAGGGC | | TTTGCCTACT | | | | GCCCTGCAC |
| | | | | | CGTGCAGAT | | | | | ACCAAGCTG | | GGGGACAGG | | | | ATGCAGGCC |
| | | | | | GTTCATCAA | | | | | GAAATCAAA | | GAACCCTGG | | | | CTGCCTCCA |
| | | | | | CACAAGC | | | | | | | TCACCGTGTC | | | | AGA |
| | | | | | | | | | | | | TGCC | | | | |
| | SEQ ID | | | 214 | 286 | 288 | 331 | 284 | 217 | 221 | 224 | 330 | 227 | 245 | 270 | 278 |
| | NO | | | | | | | | | | | | | | | |
| | | | AA | MD | NWVNVISDL | SGGGG | LLPSWA | QCTNY | MLL | DIVMTQSPDS | GGGGSG | EVQLVESGG | TTTPAPR | VAAI | ALYLLRRDQRL | RVKFSRSADA |
| | | | | WT | KKIEDLIQSM | SGGGG | ITLISVN | ALLKL | LVTS | LAVSLGERAT | GGGSGG | GLVQPGGSL | PPTPAPTI | LVLG | PPDAHKPPGGG | PAYKQGQNQ |
| | | | | WIL | HIDATLYTES | SGVTP | GIFVICC | AGDVE | LLLC | INCKSSQSLL | GGS | RLSCAASGF | ALQPLSL | LLGP | SFRTPIQEEQA | LYNELNLGRR |
| | | | | FLV | DVHPSCKVT | EPIFSL | LTYCFA | SNPGP | ELPH | YSSNQKNYL | | TFNKNAMNWV | RPEACRP | LAIL | DAHSTLAKI | EEYDVLDKR |
| | | | | AAA | AMKCFLLELQ | IGGGS | PRCRER | GSGEG | PAFL | AWYQQKPGQ | | RQAPGKGLE | AAGGAV | LGLG | | RGRDPEMGG |
| | | | | TRV | VISLESGDAS | GGGGS | RRNERL | RGSLL | LIP | PPKLLIYWAS | | WVGRIRNKT | HTRGLDF | L | | KPRRKNPQEG |
| | | | | HS | IHDTVENLII | GGGSL | RRESVR | TCGDV | | SRESGVPDRF | | NNYATYYAD | ACD | | | LYNELQKDK |
| | | | | | LANNSLSSNG | Q | PV | EENPG | | SGSGSGTDFT | | SVKARFTISR | | | | MAEAYSEIG |
| | | | | | NVTESGCKEC | | | P | | LTISSLQAED | | DDSKNSLYLQ | | | | MKGERRRGK |
| | | | | | EELEEKNIKE | | | | | VAVYYCQQY | | MNSLKTEDT | | | | GHDGLYQGL |
| | | | | | FLQSFVHIVQ | | | | | YNYPLTFGQG | | AVYYCVAGN | | | | STATKDTYD |
| | | | | | MFINTS | | | | | TKLEIK | | SFAYWGQGT | | | | ALHMQALPP |
| | | | | | | | | | | | | LVTVSA | | | | R |
| | SEQ ID | | | 218 | 285 | 287 | 219 | 283 | 216 | 208 | 223 | 206 | 226 | 244 | 269 | 277 |
| | NO | | | | | | | | | | | | | | | |
| SB062 | | | | IgE | IL15 | LR1 | hPY7 | CD8FA | B7-1 | E2A/ | hPY7 | (GGGGS)3 | hPY7 | CD8FA | CD28 | CD3z |
| 55 | | | | | | split | VL | | | T2A | VL | (SEQ | VL | | | |
| | | | | | | term | | | | | | ID NO: | | | | |
| | | | | | | linker | | | | | | 223) | | | | |
| | | | | | | + Tace10 | | | | | | | | | | |
| | IgE | | DNA | ATG | AATTGGGTC | TCTGG | CTGCTG | CAGTG | ATG | GAAGTGCAG | GGCGG | GACATCGTG | GGCGCC | ATCT | CGGAGCAAGA | AGAGTGAAG |
| | (SS) | | | GAC | AACGTGATC | CGGCG | CCTAGC | TACCA | CTGC | CTGGTGGAA | CGGAG | ATGACACAG | CTGAGC | ACA | GAAGCAGACT | TTCAGCAGA |
| | - | | | TGG | AGCGACCTG | GAGGA | TGGGCC | ACTAC | TGCT | TCTGGCGGA | GAAGC | AGCCCCGAT | AACAGC | TCTG | GCTGCACAGCG | TCCGCCGAT |
| | IL15 | | | ACC | AAGAAGATC | TCTGG | ATCACA | GCCCT | GGT | GGACTGGTT | GGAGG | AGCCTGGCC | ATCATGT | GGC | ACTACATGAAC | GCTCCCGCCT |
| | Tace10 | | | TGG | GAGGACCTG | CGGAG | CTGATC | GCTGA | CAC | CAACCTGGC | CGGAG | GTGTCTCTGG | ACTTCA | CCCT | ATGACCCCTAG | ATCAGCAGG |
| | (cleavage | | | ATC | ATCCAGAGC | GTGGA | TCCGTG | AACTG | ATCT | GGCTCTCTG | GATCCG | GAGAAAGAG | GCCACTT | CTG | ACGGCCCGGAC | GACAGAACC |
| | site) | | | CTG | ATGCACATC | AGCGG | AACGG | GCCGG | CTGC | AGACTGTCTT | GTGGTG | CCACCATCA | CGTGCC | GCT | CTACCAGAAAG | AGCTGTACA |
| | - | | | TTT | GACGCCACA | AGTTA | CATCTT | CGACG | TGCT | GTGCCGCCA | GTGGAT | ACTGCAAGA | CGTGTTT | GGA | CACTACCAGCC | ACGAGCTGA |
| | B7- | | | CTG | CTGTACACC | CACCC | CGTGAT | TGGAA | GTG | GCGGCTTCA | CT | GCAGCCAGA | CTGCCC | ACA | TTACGCTCCTC | ACCTGGGGA |
| | 1 | | | GTG | GAGAGCGAC | GAGCC | CTGCTG | TCTAA | CGA | CCTTCAACA | | GCCTGCTGT | GCCAAG | TGTG | CTAGAGACTTC | GAAGAGAAG |
| | (TM) | | | GCC | GTGCACCCT | TATCT | CCTGAC | TCCTG | GCT | AGAACGCCA | | ACTCCAGCA | CCTACA | GTGT | GCCGCCTACCG | AGTACGACG |
| | - | | | GCT | AGCTGTAAA | TCAGC | CTACTG | GACCT | GCC | TGAACTGGG | | ACCAGAAGA | ACAACC | CCTG | GTCC | TGCTGGACA |
| | E2A | | | GCC | GTGACCGCC | CTGAT | CTTCGC | GGATC | CCAT | TCCGACAGG | | ACTACCTGG | CCTGCTC | CTGC | | AGCGGAGAG |
| | T2A | | | ACA | ATGAAGTGC | CGGAG | CCCTAG | TGGCG | CCTG | CCCCTGGCA | | CCTGGTATC | CTAGAC | TGA | | GCAGAGATC |
| | - | | | AGA | TTTCTGCTGG | GCGGT | ATGCAG | AGGGA | CCTT | AAGGCCTTG | | AGCAAAAGC | CTCCTAC | GCCT | | CTGAGATGG |
| | GM- | | | GTG | AACTGCAAG | AGCGG | AGAGC | CGCGG | TCTG | AATGGGTCG | | CCGGCCAGC | ACCAGC | IGGT | | GCGGCAAGC |
| | CSF-Ra | | | CAC | TGATCAGCC | AGGCG | GGCGG | GAGTC | CTG | GACGGATCC | | CTCCTAAGCT | TCCTACA | CATC | | CCAGACGGA |
| | (SS) | | | AGC | TGGAAAGCG | GAGGA | AGAAA | TACTG | ATCC | GGAACAAGA | | GCTGATCTAT | ATCGCC | ACC | | AGAATCCTC |
| | - | | | | GCGACGCCA | AGTGG | CGAAC | ACGTG | CT | CCAACAACT | | TGGGCCAGC | AGCCAG | CTGT | | AAGAGGGCC |
| | aGPC3 | | | | GCATCCACG | TGGCG | GGCTGA | TGGAG | | ACGCCACCT | | TCCAGAGAA | CCTCTGT | ACT | | TGTATAATG |
| | hPY7 | | | | ACACCGTGG | GATCT | GAAGA | ACGTG | | ACTACGCCG | | AGCGGCGTG | CTCTGA | GCA | | AGCTGCAGA |
| | vL | | | | AAAACCTGA | CTGCA | GAATCT | GAGGA | | ACAGCGTGA | | CCCGATAGA | GGCCAG | ACC | | AAGACAAGA |
| | (GGGGS) | | | | TCATCCTGGC | A | GTGCGG | AAACC | | AGGCCAGGT | | TTTTCTGGCT | AAGCTT | ACC | | TGGCCGAGG |
| | 3(SEQ | | | | CAACAACAG | | CCCGTT | CTGGA | | TCACCATCTC | | CTGGCAGCG | GTAGAC | GG | | CCTACAGCG |
| | ID | | | | CCTGAGCAG | | | CCT | | CAGAGATGA | | GCACCGACT | CTGCTGC | | | AGATCGGAA |
| | NO: | | | | CAACGGCAA | | | | | CAGCAAGAA | | TCACCCTGA | AGGCGG | | | TGAAGGGCG |
| | 223) | | | | TGTGACCGA | | | | | CAGCCTGTA | | CAATTTCTAG | AGCCGT | | | AGCGCAGAA |
| | - | | | | GTCCGGCTG | | | | | CCTGCAGAT | | CCTGCAAGC | GCATAC | | | GAGGCAAGG |
| | aGPC3 | | | | CAAAGAGTG | | | | | GAACTCCCT | | CGAGGACGT | AAGAGG | | | GACACGATG |
| | hPY7 | | | | CGAGGAACT | | | | | GAAAACCGA | | GGCCGTGTA | ACTGGA | | | GACTGTACC |
| | vH | | | | GGAAGAGAA | | | | | GGACACCGC | | TTACTGCCA | TTTCGCC | | | AGGGCCTGA |
| | - | | | | GAATATCAA | | | | | CGTGTACTAT | | GCAGTACTA | TGCGAC | | | GCACCGCCA |
| | CD8FA | | | | AGAGTTCCT | | | | | TGCGTGGCC | | CAACTACCC | | | | CCAAGGATA |
| | (Hinge) | | | | GCAGAGCTT | | | | | GGCAATAGC | | TCTGACCTTC | | | | CCTATGATG |
| | - | | | | CGTGCACAT | | | | | TTTGCCTACT | | GGCCAGGGC | | | | CCCTGCACA |
| | CDSFA | | | | CGTGCAGAT | | | | | GGGGACAGG | | ACCAAGCTG | | | | TGCAGGCCC |
| | (TM) | | | | GTTCATCAA | | | | | GCACCCTGG | | GAAATCAAA | | | | TGCCTCCAA |
| | - | | | | CACAAGC | | | | | TTACAGTTTC | | | | | | GA |
| | CD28 | | | | | | | | | TGCT | | | | | | |
| | (ICD) | | | | | | | | | | | | | | | |
| | - | | | | | | | | | | | | | | | |
| | CD3z | | | | | | | | | | | | | | | |
| | (ICD) | | | | | | | | | | | | | | | |
| | SEQ ID | | | 214 | 286 | 288 | 331 | 284 | 217 | 222 | 332 | 229 | 243 | 244 | 268 | 280 |
| | NO | | | | | | | | | | | | | | | |
| | | | AA | MD | NWVNVISDL | SGGGG | LLPSWA | QCTNY | MLL | | GGGGSG | DIVMTQSPDS | GALSNSI | IYIW | RSKRSRLLHSD | RVKFSRSADA |
| | | | | WT | KKIEDLIQSM | SGGGG | ITLISVN | ALLKL | LVTS | | GGGSGG | LAVSLGERAT | MYFSHF | APLA | YMNMTPRRPGP | PAYQQGQNQ |
| | | | | WIL | HIDATLYTES | SGVTP | GIFVICC | AGDVE | LLLC | | GGS | INCKSSQSLL | VPVFLPA | GTC | TRKHYQPYAPP | LYNELNLGRR |
| | | | | FLV | DVHPSCKVT | EPIFSL | LTYCFA | SNPGP | ELPH | | | YSSNQKNYL | KPTTTPA | GVLL | RDFAAYRS | EEYDVLDKR |
| | | | | AAA | AMKCFLLELQ | IGGGS | PRCRER | GSGEG | PAFL | | | AWYQQKPGQ | PRPPTPA | LSLV | | RGRDPEMGG |
| | | | | HS | VISLESGDAS | GGGGS | RRNERL | RGSLL | LIP | | | PPKLLIYWAS | PTIASQPL | ITLY | | KPRRKNPQEG |
| | | | | TRV | IHDTVENLII | GGGSL | RRESVR | TCGDV | | | | SRESGVPDRF | SLRPEAC | CNH | | LYNELQKDK |
| | | | | | LANNSLSSNG | Q | PV | EENPG | | | | SGSGSGTDFT | RPAAGG | R | | MAEAYSEIG |
| | | | | | NVTESGCKEC | | | P | | | | LTISSLQAED | AVHTRG | | | MKGERRRGK |
| | | | | | EELEEKNIKE | | | | | | | VAVYYCQQY | LDFACD | | | GHDGLYQGL |
| | | | | | FLQSFVHIVQ | | | | | | | YNYPLTFGQG | | | | STATKDTYD |
| | | | | | MFINTS | | | | | | | TKLEIK | | | | ALHMQALPP |
| | | | | | | | | | | | | | | | | R |
| | SEQ ID | | | 218 | 285 | 287 | 219 | 283 | 216 | 206 | 223 | 208 | 228 | 242 | 267 | 279 |
| | NO | | | | | | | | | | | | | | | |
| SB062 | | | | IgE | IL15 | LR1 | B7-1 | E2A/ | GM- | hPY7 VL | (GGGGS)3 | hPY7 VH | CD8 S2L | OX40 | OX40 | CD3zmut |
| 94 | | | | | split N | | | T2A | CSF- | | (SEQ ID | | | | | |
| | | | | | term | | | | | | NO: | | | | | |
| | | | | | Ra | | | | | | 223) | | | | | |
| | | | | | linker | | | | | | | | | | | |
| | | | | | Tace10 | | | | | | | | | | | |
| | IgE(SS)- | Retro | DNA | ATG | AATTGGGTC | TCTGG | TGGGCC | CAGTG | ATG | GACATCGTG | GGCGG | TCAGGTGGC | ACAACA | GCC | GCTCTGTATCT | AGAGTGAAG |
| | IL15 | Vec | | GAC | AACGTGATC | CGGCG | CTGCTG | TACCA | CTGC | ATGACACAG | CGGAG | GAAGTGCAG | ACCCCT | GTG | GCTGCGGAGG | TTCAGCAGG |
| | Tace10 | | | TGG | AGCGACCTG | GAGGA | CCTAGC | ACTAC | TGCT | AGCCCCGAT | GATCTG | CTGGTTGAA | GCTCCTA | GCC | GACCAAAGACT | AGCGCAGAC |
| | (cleavage | | | ACC | AAGAAGATC | TCTGG | ATCACA | GCCCT | GGT | AGCCTGGCC | GCGGA | GGCCTGGTT | GACCTC | ATTC | GCCTCCTGATG | GCCCCCGCG |
| | site)-B7- | | | TGG | GAGGACCTG | CGGAG | CTGATC | GCTGA | CAC | GTGTCTCTGG | GGTGG | CAACCTGGC | CTACAC | TCG | CTCACAAGCCT | TACAAGCAG |
| | 1(TM)- | | | ATC | ATCCAGAGC | GTGGA | TCCGTG | AACTG | ATCT | GAGAAAGAG | AAGTG | GGATCTCTG | CAGCTC | GAC | CCAGGCGGAG | GGCCAGAAC |
| | E2AT2A | | | CTG | ATGCACATC | AGCGG | AACGG | GCCGG | CTGC | CCACCATCA | GCGGA | AGACTGAGC | CTACAA | TGG | GCAGCTTCAGA | CAGCTCTAT |
| | -GM- | | | TTT | GACGCCACA | AGTTA | CATCTT | CGACG | TGCT | ACTGCAAGA | GGCGG | TGTGCCGCC | TCGCCCT | GAC | ACCCCTATCCA | AACGAGCTC |
| | CSF-Ra | | | CTG | CTGTACACC | CACCC | CGTGAT | TGGAA | GTG | GCAGCCAGA | ATCT | AGCGGCTTC | GCAGCC | TTGT | AGAGGAACAG | AATCTAGGA |
| | (SS)- | | | GTG | GAGAGCGAC | GAGCC | CTGCTG | TCTAA | CGA | GCCTGCTGT | | ACCTTCAAC | TCTGTCT | TCTG | GCCGACGCTCA | CGAAGAGAG |
| | aGPC3 | | | GCC | GTGCACCCT | TATCT | CCTGAC | TCCTG | GCT | ACTCCAGCA | | AAGAACGCC | CTGAGG | GGA | CAGCACCCTGG | GAGTACGAT |
| | hPY7 | | | GCT | AGCTGTAAA | TCAGC | CTACTG | GACCT | GCC | ACCAGAAGA | | ATGAACTGG | CCAGAA | CTGC | CCAAGATT | GTTTTGGAC |
| | vL | | | GCC | GTGACCGCC | CTGAT | CTTCGC | GGATC | CCAT | ACTACCTGG | | GTCCGACAG | GCTTGTA | TGG | | AAGAGACGT |
| | (GGGGS) | | | ACA | ATGAAGTGC | CGGAG | CCCTAG | TGGCG | CCTG | CCTGGTATC | | GCCCCTGGC | GACCAG | GAC | | GGCCGGGAC |
| | 3(SEQ | | | AGA | TTTCTGCTGG | GCGGT | ATGCAG | AGGGA | CCTT | AGCAAAAGC | | AAAGGCCTT | CTGCTG | CTCT | | CCTGAGATG |
| | ID NO: | | | GTG | AACTGCAAG | AGCGG | AGAGC | CGCGG | TCTG | CCGGCCAGC | | GAATGGGTC | GCGGAG | GGC | | GGGGGAAAG |
| | 223)- | | | CAC | TGATCAGCC | AGGCG | GGCGG | GAGTC | CTG | CTCCTAAGCT | | GGACGGATC | CCGTGC | CATT | | CCGAGAAGG |
| | aGPC3 | | | AGC | TGGAAAGCG | GAGGA | AGAAA | TACTG | ATCC | GCTGATCTAT | | CGGAACAAG | ATACAA | CTGC | | AAGAACCCT |
| | hPY7 | | | | GCGACGCCA | AGTGG | CGAAC | ACGTG | CT | TGGGCCAGC | | ACCAACAAC | GAGGAC | TG | | CAGGAAGGC |
| | vH | | | | GCATCCACG | TGGCG | GGCTGA | TGGAG | | TCCAGAGAA | | TACGCCACC | TGGACTT | | | CTGTACAAT |
| | -CD8 | | | | ACACCGTGG | GATCT | GAAGA | ACGTG | | AGCGGCGTG | | TACTACGCC | CGCCTGT | | | GAACTGCAG |
| | S2L | | | | AAAACCTGA | CTGCA | GAATCT | GAGGA | | CCCGATAGA | | GACAGCGTG | GATG | | | AAAGATAAG |
| | (Hinge)- | | | | TCATCCTGGC | A | GTGCGG | AAACC | | TTTTCTGGCT | | AAGGCCAGA | | | | ATGGCGGAG |
| | OX40 | | | | CAACAACAG | | CCCGTT | CTGGA | | CTGGCAGCG | | TTCACCATCA | | | | GCCTACAGT |
| | (TM)- | | | | CCTGAGCAG | | | CCT | | GCACCGACT | | GCCGGGACG | | | | GAGATTGGG |
| | OX40 | | | | CAACGGCAA | | | | | TCACCCTGA | | ACAGCAAGA | | | | ATGAAAGGC |
| | (ICD)- | | | | TGTGACCGA | | | | | CAATTTCTAG | | ACAGCCTGT | | | | GAGCGCCGG |
| | CD3zmut | | | | GTCCGGCTG | | | | | CCTGCAAGC | | ACCTGCAGA | | | | AGGGGCAAG |
| | (ICD) | | | | CAAAGAGTG | | | | | CGAGGACGT | | TGAACTCCCT | | | | GGGCACGAT |
| | | | | | CGAGGAACT | | | | | GGCCGTGTA | | GAAAACCGA | | | | GGCCTTTACC |
| | | | | | GGAAGAGAA | | | | | CTACTGCCA | | GGACACCGC | | | | AGGGTCTCA |
| | | | | | GAATATCAA | | | | | GCAGTACTA | | CGTGTATTAT | | | | GTACAGCCA |
| | | | | | AGAGTTCCT | | | | | CAACTACCC | | TGCGTGGCC | | | | CCAAGGACA |
| | | | | | GCAGAGCTT | | | | | TCTGACCTTC | | GGCAACAGC | | | | CCTACGACG |
| | | | | | CGTGCACAT | | | | | GGCCAGGGC | | TTTGCCTACT | | | | CCCTTCACAT |
| | | | | | CGTGCAGAT | | | | | ACCAAGCTG | | GGGGACAGG | | | | GCAGGCCCT |
| | | | | | GTTCATCAA | | | | | GAAATCAAA | | GAACCCTGG | | | | GCCCCCTCG |
| | | | | | CACAAGC | | | | | | | TCACCGTGTC | | | | C |
| | | | | | | | | | | | | TGCC | | | | |
| | SEQ ID | | | 214 | 286 | 288 | 331 | 284 | 217 | 221 | 224 | 330 | 352 | 245 | 270 | 334 |
| | NO | | | | | | | | | | | | | | | |
| | | | AA | MD | NWVNVISDL | SGGGG | LLPSWA | QCTNY | MLL | DIVMTQSPDS | GGGGSG | EVQLVESG | TTTPAPR | VAAI | ALYLLRRDQRL | RVKFSRSADA |
| | | | | WT | KKIEDLIQSM | SGGGG | ITLISVN | ALLKL | LVTS | LAVSLGERAT | GGGSGG | GGLVQPGG | PPTPAPTI | LGLG | PPDAHKPPGGG | PAYKQGQNQ |
| | | | | WIL | HIDATLYTES | SGVTP | GIFVICC | AGDVE | LLLC | INCKSSQSLL | GGS | SLRLSCAA | ALQPLSL | LVLG | SFRTPIQEEQA | LYNELNLGRR |
| | | | | FLV | DVHPSCKVT | EPIFSL | LTYCFA | SNPGP | ELPH | YSSNQKNYL | | SGFTFNKN | RPEACRP | LLGP | DAHSTLAKI | EEYDVLDKR |
| | | | | AAA | AMKCFLLEL | IGGGS | PRCRER | GSGEG | PAFL | AWYQQKPGQ | | AMNWVRQA | AAGGAV | LAIL | | RGRDPEMGG |
| | | | | TRV | QVISLESGDA | GGGGS | RRNERL | RGSLL | LIP | PPKLLIYWAS | | PGKGLEWV | HTRGLDF | L | | KPRRKNPQEG |
| | | | | HS | SIHDTVENLII | GGGSL | RRESVR | TCGDV | | SRESGVPDRF | | GRIRNKTN | ACD | | | LYNELQKDK |
| | | | | | LANNSLSSNG | Q | PV | EENPG | | SGSGSGTDFT | | NYATYYAD | | | | MAEAYSEIG |
| | | | | | NVTESGCKEC | | | P | | LTISSLQAED | | SVKARFTI | | | | MKGERRRGK |
| | | | | | EELEEKNIKE | | | | | VAVYYCQQY | | SRDDSKNS | | | | GHDGLYQGL |
| | | | | | FLQSFVHIVQ | | | | | YNYPLTFGQG | | LYLQMNSL | | | | STATKDTYD |
| | | | | | MFINTS | | | | | TKLEIK | | KTEDTAVY | | | | ALHMQALPP |
| | | | | | | | | | | | | YCVAGNSF | | | | R |
| | | | | | | | | | | | | AYWGQGTL | | | | |
| | | | | | | | | | | | | VTVSA | | | | |
| | SEQ ID | | | 218 | 285 | 287 | 219 | 283 | 216 | 208 | 223 | 206 | 226 | 244 | 269 | 277 |
| | NO | | | | | | | | | | | | | | | |
| SB06 | | | | IgE | IL15 | TaceOP | B7-1 | E2A/T2 | GM- | hPY7 | (GGGGS) | hPY7 | CD8S2L | OX40 | OX40 | CD3z |
| 692 | | | | | | T + LR1 | | A | CSF- | VL | 3(SEQ | VH | | | | |
| | | | | | | linker | | | Ra | | ID NO: | | | | | |
| | | | | | | | | | | | 223) | | | | | |
| | IgE(SS)- | Sin | DNA | ATG | AATTGGGTC | CCCAG | CTGCTG | CAGTG | ATG | GACATCGTG | GGCGG | GAAGTGCAG | ACAACA | GTG | GCTCTGTATCT | AGAGTGAAG |
| | IL15- | Vec | | GAC | AACGTGATC | AGCCG | CCTAGC | TACCA | CTGC | ATGACACAG | CGGAG | CTGGTTGAA | ACCCCT | GCC | GCTGCGGAGG | TTCAGCAGA |
| | TaceOPT | | | TGG | AGCGACCTG | AGGCT | TGGGCC | ACTAC | TGCT | AGCCCCGAT | GATCTG | TCAGGTGGC | GCTCCTA | GCC | GACCAAAGACT | AGCGCCGAC |
| | (cleavage | | | ACC | AAGAAGATC | CTGAA | ATCACA | GCCCT | GGT | AGCCTGGCC | GCGGA | GGCCTGGTT | GACCTC | ATTC | GCCTCCTGATG | GCACCCGCC |
| | site)-B7- | | | TGG | GAGGACCTG | AGGCG | CTGATC | GCTGA | CAC | GTGTCTCTGG | GGTGG | CAACCTGGC | CTACAC | TCG | CTCACAAGCCT | TATAAGCAG |
| | 1(TM)- | | | ATC | ATCCAGAGC | GATCA | TCCGTG | AACTG | ATCT | GAGAAAGAG | AAGTG | GGATCTCTG | CAGCTC | GAC | CCAGGCGGAG | GGACAGAAC |
| | E2AT2A | | | CTG | ATGCACATC | GGCGG | AACGG | GCCGG | CTGC | CCACCATCA | GCGGA | AGACTGAGC | CTACAA | TGG | GCAGCTTCAGA | CAGCTGTAC |
| | -GM- | | | TTT | GACGCCACA | CGGTG | CATCTT | CGACG | TGCT | ACTGCAAGA | GGCGG | TGTGCCGCC | TCGCCCT | GAC | ACCCCTATCCA | AACGAGCTG |
| | CSF-Ra | | | CTG | CTGTACACC | GTAGT | CGTGAT | TGGAA | GTG | GCAGCCAGA | ATCT | AGCGGCTTC | GCAGCC | TTGT | AGAGGAACAG | AACCTGGGG |
| | (SS)- | | | GTG | GAGAGCGAC | GGAGG | CTGCTG | TCTAA | CGA | GCCTGCTGT | | ACCTTCAAC | TCTGTCT | TCTG | GCCGACGCTCA | AGAAGAGAA |
| | aGPC3 | | | GCC | GTGCACCCT | CGGAG | CCTGAC | TCCTG | GCT | ACTCCAGCA | | AAGAACGCC | CTGAGG | GGA | CAGCACCCTGG | GAGTACGAC |
| | hPY7 vL | | | GCT | AGCTGTAAA | GCTCA | CTACTG | GACCT | GCC | ACCAGAAGA | | ATGAACTGG | CCAGAA | CTGC | CCAAGATT | GTGCTGGAC |
| | (GGGGS) | | | GCC | GTGACCGCC | GGCGG | CTTCGC | GGATC | CCAT | ACTACCTGG | | GTCCGACAG | GCTTGTA | TGG | | AAGCGGAGA |
| | 3(SEQ | | | ACA | ATGAAGTGC | CGGAG | CCCTAG | TGGCG | CCTG | CCTGGTATC | | GCCCCTGGC | GACCAG | GAC | | GGCAGAGAT |
| | ID NO: | | | AGA | TTTCTGCTGG | GTTCC | ATGCAG | AGGGA | CCTT | AGCAAAAGC | | AAAGGCCTT | CTGCTG | CTCT | | CCTGAGATG |
| | 223)- | | | GTG | AACTGCAAG | GGAGG | AGAGC | CGCGG | TCTG | CCGGCCAGC | | GAATGGGTC | GCGGAG | GGC | | GGCGGCAAG |
| | aGPC3 | | | CAC | TGATCAGCC | TGGCG | GGCGG | GAGTC | CTG | CTCCTAAGCT | | GGACGGATC | CCGTGC | CATT | | CCCAGACGG |
| | hPY7 vH | | | AGC | TGGAAAGCG | GTTCC | AGAAA | TACTG | ATCC | GCTGATCTAT | | CGGAACAAG | ATACAA | CTGC | | AAGAATCCT |
| | -CD8 | | | | GCGACGCCA | GGCGG | CGAAC | ACGTG | CT | TGGGCCAGC | | ACCAACAAC | GAGGAC | TG | | CAAGAGGGC |
| | (Hinge)- | | | | GCATCCACG | AGGAT | GGCTGA | TGGAG | | TCCAGAGAA | | TACGCCACC | TGGACTT | | | CTGTATAAT |
| | OX40 | | | | ACACCGTGG | CTCTT | GAAGA | ACGTG | | AGCGGCGTG | | TACTACGCC | CGCCTGT | | | GAGCTGCAG |
| | (TM)- | | | | AAAACCTGA | CAAT | GAATCT | GAGGA | | CCCGATAGA | | GACAGCGTG | GAT | | | AAAGACAAG |
| | OX40 | | | | TCATCCTGGC | | GTGCGG | AAACC | | TTTTCTGGCT | | AAGGCCAGA | | | | ATGGCCGAG |
| | (ICD)- | | | | CAACAACAG | | CCCGTT | CTGGA | | CTGGCAGCG | | TTCACCATCA | | | | GCCTACAGC |
| | CD3z | | | | CCTGAGCAG | | | CCT | | GCACCGACT | | GCCGGGACG | | | | GAGATCGGA |
| | (ICD) | | | | CAACGGCAA | | | | | TCACCCTGA | | ACAGCAAGA | | | | ATGAAGGGC |
| | | | | | TGTGACCGA | | | | | CAATTTCTAG | | ACAGCCTGT | | | | GAGCGCAGA |
| | | | | | GTCCGGCTG | | | | | CCTGCAAGC | | ACCTGCAGA | | | | AGAGGCAAG |
| | | | | | CAAAGAGTG | | | | | CGAGGACGT | | TGAACTCCCT | | | | GGACACGAT |
| | | | | | CGAGGAACT | | | | | GGCCGTGTA | | GAAAACCGA | | | | GGACTGTAC |
| | | | | | GGAAGAGAA | | | | | CTACTGCCA | | GGACACCGC | | | | CAGGGCCTG |
| | | | | | GAATATCAA | | | | | GCAGTACTA | | CGTGTATTAT | | | | AGCACCGCC |
| | | | | | AGAGTTCCT | | | | | CAACTACCC | | TGCGTGGCC | | | | ACCAAGGAT |
| | | | | | GCAGAGCTT | | | | | TCTGACCTTC | | GGCAACAGC | | | | ACCTATGAT |
| | | | | | CGTGCACAT | | | | | GGCCAGGGC | | TTTGCCTACT | | | | GCCCTGCAC |
| | | | | | CGTGCAGAT | | | | | ACCAAGCTG | | GGGGACAGG | | | | ATGCAGGCC |
| | | | | | GTTCATCAA | | | | | GAAATCAAA | | GAACCCTGG | | | | CTGCCTCCA |
| | | | | | CACAAGC | | | | | | | TCACCGTGTC | | | | AGA |
| | | | | | | | | | | | | TGCC | | | | |
| | SEQ ID | | | 214 | 286 | 292 | 331 | 284 | 217 | 221 | 224 | 330 | 227 | 245 | 270 | 278 |
| | NO | | | | | | | | | | | | | | | |
| | | | AA | MD | NWVNVISDL | PRAEA | LLPSWA | QCTNY | MLL | DIVMTQSPDS | GGGGSG | EVQLVESGG | TTTPAPR | VAAI | ALYLLRRDQRL | RVKFSRSADA |
| | | | | WT | KKIEDLIQSM | LKGGS | ITLISVN | ALLKL | LVTS | LAVSLGERAT | GGGSGG | GLVQPGGS | PPTPAPTI | LGLG | PPDAHKPPGGG | PAYKQGQNQ |
| | | | | WIL | HIDATLYTES | GGGGS | GIFVICC | AGDVE | LLLC | INCKSSQSLL | GGS | LRLSCAAS | ALQPLSL | LVLG | SFRTPIQEEQA | LYNELNLGRR |
| | | | | FLV | DVHPSCKVT | GGGGS | LTYCFA | SNPGP | ELPH | YSSNQKNYL | | GFTFNKNA | RPEACRP | LLGP | DAHSTLAKI | EEYDVLDKR |
| | | | | AAA | AMKCFLLELQ | GGGGS | PRCRER | GSGEG | PAFL | AWYQQKPGQ | | MNWVRQAP | AAGGAV | LAIL | | RGRDPEMGG |
| | | | | TRV | VISLESGDAS | GGGGS | RRNERL | RGSLL | LIP | PPKLLIYWAS | | GKGLEWVG | HTRGLDF | L | | KPRRKNPQEG |
| | | | | HS | IHDTVENLII | GGGSL | RRESVR | TCGDV | | SRESGVPDRF | | RIRNKTNN | ACD | | | LYNELQKDK |
| | | | | | LANNSLSSNG | Q | PV | EENPG | | SGSGSGTDFT | | YATYYADS | | | | MAEAYSEIG |
| | | | | | NVTESGCKEC | | | P | | LTISSLQAED | | VKARFTIS | | | | MKGERRRGK |
| | | | | | EELEEKNIKE | | | | | VAVYYCQQY | | RDDSKNSL | | | | GHDGLYQGL |
| | | | | | FLQSFVHIVQ | | | | | YNYPLTFGQG | | YLQMNSLK | | | | STATKDTYD |
| | | | | | MFINTS | | | | | TKLEIK | | TEDTAVYY | | | | ALHMQALPP |
| | | | | | | | | | | | | CVAGNSFA | | | | R |
| | | | | | | | | | | | | YWGQGTLV | | | | |
| | | | | | | | | | | | | TVSA | | | | |
| | SEQ ID | | | 218 | 285 | 289 | 219 | 283 | 216 | 208 | 223 | 206 | 226 | 244 | 269 | 277 |
| | NO | | | | | | | | | | | | | | | |
| SB | | | | IgE | IL15 | Tace | B7-1 | E2A/T2 | GM- | hPY7 | (GGGGS) | hPY7 | CD8FA | CD8F | CD28 | CD3z |
| 06261 | | | | | | OPT | | A | CSF- | VH | 3(SEQ | VL | | A | | |
| | | | | | | LR1 | | | Ra | | ID NO: | | | | | |
| | | | | | | linker | | | | | 223) | | | | | |
| | IgE(SS)- | Sin | DNA | ATG | AATTGGGTC | TCTGG | CTGCTG | CAGTG | ATG | GAAGTGCAG | GGCGG | GACATCGTG | GGCGCC | ATCT | CGGAGCAAGA | AGAGTGAAG |
| | IL15 | Vec | | GAC | AACGTGATC | CGGCG | CCTAGC | TACCA | CTGC | CTGGTGGAA | CGGAG | ATGACACAG | CTGAGC | ACA | GAAGCAGACT | TTCAGCAGA |
| | Tace10 | | | TGG | AGCGACCTG | GAGGA | TGGGCC | ACTAC | TGCT | TCTGGCGGA | GAAGC | AGCCCCGAT | AACAGC | TCTG | GCTGCACAGCG | TCCGCCGAT |
| | (cleavage | | | ACC | AAGAAGATC | TCTGG | ATCACA | GCCCT | GGT | GGACTGGTT | GGAGG | AGCCTGGCC | ATCATGT | GGC | ACTACATGAAC | GCTCCCGCCT |
| | site)-B7- | | | TGG | GAGGACCTG | CGGAG | CTGATC | GCTGA | CAC | CAACCTGGC | CGGAG | GTGTCTCTGG | ACTTCA | CCCT | ATGACCCCTAG | ATCAGCAGG |
| | 1(TM)- | | | ATC | ATCCAGAGC | GTGGA | TCCGTG | AACTG | ATCT | GGCTCTCTG | GATCCG | GAGAAAGAG | GCCACTT | CTG | ACGGCCCGGAC | GACAGAACC |
| | E2AT2A | | | CTG | ATGCACATC | AGCGG | AACGG | GCCGG | CTGC | AGACTGTCTT | GTGGTG | CCACCATCA | CGTGCC | GCT | CTACCAGAAAG | AGCTGTACA |
| | -GM- | | | TTT | GACGCCACA | AGTTA | CATCTT | CGACG | TGCT | GTGCCGCCA | GTGGAT | ACTGCAAGA | CGTGTTT | GGA | CACTACCAGCC | ACGAGCTGA |
| | CSF-Ra | | | CTG | CTGTACACC | CACCC | CGTGAT | TGGAA | GTG | GCGGCTTCA | CT | GCAGCCAGA | CTGCCC | ACA | TTACGCTCCTC | ACCTGGGGA |
| | (SS)- | | | GTG | GAGAGCGAC | GAGCC | CTGCTG | TCTAA | CGA | CCTTCAACA | | GCCTGCTGT | GCCAAG | TGTG | CTAGAGACTTC | GAAGAGAAG |
| | aGPC3 | | | GCC | GTGCACCCT | TATCT | CCTGAC | TCCTG | GCT | AGAACGCCA | | ACTCCAGCA | CCTACA | GTGT | GCCGCCTACCG | AGTACGACG |
| | hPY7 vL | | | GCT | AGCTGTAAA | TCAGC | CTACTG | GACCT | GCC | TGAACTGGG | | ACCAGAAGA | ACAACC | CCTG | GTCC | TGCTGGACA |
| | (GGGGS) | | | GCC | GTGACCGCC | CTGAT | CTTCGC | GGATC | CCAT | TCCGACAGG | | ACTACCTGG | CCTGCTC | CTGC | | AGCGGAGAG |
| | 3(SEQ | | | ACA | ATGAAGTGC | CGGAG | CCCTAG | TGGCG | CCTG | CCCCTGGCA | | CCTGGTATC | CTAGAC | TGA | | GCAGAGATC |
| | ID NO: | | | AGA | TTTCTGCTGG | GCGGT | ATGCAG | AGGGA | CCTT | AAGGCCTTG | | AGCAAAAGC | CTCCTAC | GCCT | | CTGAGATGG |
| | 223)- | | | GTG | AACTGCAAG | AGCGG | AGAGC | CGCGG | TCTG | AATGGGTCG | | CCGGCCAGC | ACCAGC | GGT | | GCGGCAAGC |
| | aGPC3 | | | CAC | TGATCAGCC | AGGCG | GGCGG | GAGTC | CTG | GACGGATCC | | CTCCTAAGCT | TCCTACA | CATC | | CCAGACGGA |
| | hPY7 vH | | | AGC | TGGAAAGCG | GAGGA | AGAAA | TACTG | ATCC | GGAACAAGA | | GCTGATCTAT | ATCGCC | ACC | | AGAATCCTC |
| | -CD8FA | | | | GCGACGCCA | AGTGG | CGAAC | ACGTG | CT | CCAACAACT | | TGGGCCAGC | AGCCAG | CTGT | | AAGAGGGCC |
| | (Hinge)- | | | | GCATCCACG | TGGCG | GGCTGA | TGGAG | | ACGCCACCT | | TCCAGAGAA | CCTCTGT | ACT | | TGTATAATG |
| | CD8FA | | | | ACACCGTGG | GATCT | GAAGA | ACGTG | | ACTACGCCG | | AGCGGCGTG | CTCTGA | GCA | | AGCTGCAGA |
| | (TM)- | | | | AAAACCTGA | CTGCA | GAATCT | GAGGA | | ACAGCGTGA | | CCCGATAGA | GGCCAG | ACC | | AAGACAAGA |
| | CD28 | | | | TCATCCTGGC | A | GTGCGG | AAACC | | AGGCCAGGT | | TTTTCTGGCT | AAGCTT | ACC | | TGGCCGAGG |
| | (ICD)- | | | | CAACAACAG | | CCCGTT | CTGGA | | TCACCATCTC | | CTGGCAGCG | GTAGAC | GG | | CCTACAGCG |
| | CD3z | | | | CCTGAGCAG | | | CCT | | CAGAGATGA | | GCACCGACT | AGCCGT | | | AGATCGGAA |
| | (ICD) | | | | CAACGGCAA | | | | | CAGCAAGAA | | TCACCCTGA | CTGCTG | | | TGAAGGGCG |
| | | | | | TGTGACCGA | | | | | CAGCCTGTA | | CAATTTCTAG | CAGGCGG | | | AGCGCAGAA |
| | | | | | GTCCGGCTG | | | | | CCTGCAGAT | | CCTGCAAGC | GCATAC | | | GAGGCAAGG |
| | | | | | CAAAGAGTG | | | | | GAACTCCCT | | CGAGGACGT | AAGAGG | | | GACACGATG |
| | | | | | CGAGGAACT | | | | | GAAAACCGA | | GGCCGTGTA | ACTGGA | | | GACTGTACC |
| | | | | | GGAAGAGAA | | | | | GGACACCGC | | TTACTGCCA | TTTCGCC | | | AGGGCCTGA |
| | | | | | GAATATCAA | | | | | CGTGTACTAT | | GCAGTACTA | TGCGAC | | | GCACCGCCA |
| | | | | | AGAGTTCCT | | | | | TGCGTGGCC | | CAACTACCC | | | | CCAAGGATA |
| | | | | | GCAGAGCTT | | | | | GGCAATAGC | | TCTGACCTTC | | | | CCTATGATG |
| | | | | | CGTGCACAT | | | | | TTTGCCTACT | | GGCCAGGGC | | | | CCCTGCACA |
| | | | | | CGTGCAGAT | | | | | GGGGACAGG | | ACCAAGCTG | | | | TGCAGGCCC |
| | | | | | GTTCATCAA | | | | | GCACCCTGG | | GAAATCAAA | | | | TGCCTCCAA |
| | | | | | CACAAGC | | | | | TTACAGTTTC | | | | | | GA |
| | | | | | | | | | | TGCT | | | | | | |
| | SEQ ID | | | 214 | 286 | 288 | 331 | 284 | 217 | 222 | 332 | 333 | 229 | 243 | 268 | 280 |
| | NO | | | | | | | | | | | | | | | |
| | | | AA | MD | NWVNVISDL | SGGGG | LLPSWA | QCTNY | IMLL | EVQLVESGG | GGGGSG | DIVMTQSPDS | GALSNSI | IYIW | RSKRSRLLHSD | RVKFSRSADA |
| | | | | WT | KKIEDLIQSM | SGGGG | ITLISVN | ALLKL | LVTS | GLVQPGGSLR | GGGSGG | LAVSLGERAT | MYFSHF | APLA | YMNMTPRRPGP | PAYQQGQNQ |
| | | | | WIL | HIDATLYTES | SGVTP | GIFVICC | AGDVE | LLLC | LSCAASGFTF | GGS | INCKSSQSLL | VPVFLPA | GTC | TRKHYQPYAPP | LYNELNLGRR |
| | | | | FLV | DVHPSCKVT | EPIFSL | LTYCFA | SNPGP | ELPH | NKNAMNWV | | YSSNQKNYL | KPTTTPA | GVLL | RDFAAYRS | EEYDVLDKR |
| | | | | AAA | AMKCFLLEL | IGGGS | PRCRER | GSGEG | PAFL | RQAPGKGLE | | AWYQQKPGQ | PRPPTPA | LSLV | | RGRDPEMGG |
| | | | | TRV | QVISLESGDA | GGGGS | RRNERL | RGSLL | LIP | WVGRIRNKT | | PPKLLIYWAS | PTIASQPL | ITLY | | KPRRKNPQEG |
| | | | | HS | SIHDTVENLI | GGGSL | RRESVR | TCGDV | | NNYATYYAD | | SRESGVPDRF | SLRPEAC | CNHR | | LYNELQKDK |
| | | | | | ILANNSLSSN | Q | PV | EENPG | | SVKARFTISR | | SGSGSGTDFT | RPAAGG | | | MAEAYSEIG |
| | | | | | GNVTESGCKEC | | | P | | DDSKNSLYLQ | | LTISSLQAED | AVHTRG | | | MKGERRRGK |
| | | | | | EELEEKNIKE | | | | | MNSLKTEDT | | VAVYYCQQY | LDFACD | | | GHDGLYQGL |
| | | | | | FLQSFVHIVQ | | | | | AVYYCVAGN | | YNYPLTFGQ | | | | STATKDTYD |
| | | | | | MFINTS | | | | | SFAYWGQGT | | GTKLEIK | | | | ALHMQALPP |
| | | | | | | | | | | LVTVSA | | | | | | R |
| | SEQ ID | | | 218 | 285 | 287 | 219 | 283 | 216 | 206 | 223 | 208 | 228 | 242 | 267 | 279 |
| | NO | | | | | | | | | | | | | | | |
| SB | | | | IgE | IL15 | TaceOP | B7-1 | E2A/ | GM- | hPY7 | (GGGGS) | hPY7 | CD8FA | CD8F | CD28 | CD3z |
| 05009 | | | | | | T + LR1 | | T2A | CSF- | VH | 3(SEQ | VL | | A | | |
| | | | | | | linker | | | Ra | | ID NO: | | | | | |
| | | | | | | | | | | | 223) | | | | | |
| | (IgE(SS) | Sin | DNA | ATG | AATTGGGTC | TCTGG | CTGCTG | None | ATG | GAAGTGCAG | GGCGG | GACATCGTG | GCCCTG | ATCT | CGGAGCAAGA | AGAGTGAAG |
| | -IL15 | Vec | | GAC | AACGTGATC | CGGCG | CCTAGC | | CTGC | CTGGTGGAA | CGGAG | ATGACACAG | AGCAAC | ACA | GAAGCAGACT | TTCAGCAGA |
| | Tace10 | | | TGG | AGCGACCTG | GAGGA | TGGGCC | | TGCT | TCTGGCGGA | GAAGC | AGCCCCGAT | AGCATC | TCTG | GCTGCACAGCG | TCCGCCGAT |
| | (cleavage | | | ACC | AAGAAGATC | TCTGG | ATCACA | | GGT | GGACTGGTT | GGAGG | AGCCTGGCC | ATGTACT | GGC | ACTACATGAAC | GCTCCCGCCT |
| | site)-B7- | | | TGG | GAGGACCTG | CGGAG | CTGATC | | CAC | CAACCTGGC | CGGAG | GTGTCTCTGG | TCAGCC | CCCT | ATGACCCCTAG | ATCAGCAGG |
| | 1(TM)) | | | ATC | ATCCAGAGC | GTGGA | TCCGTG | | ATCT | GGCTCTCTG | GATCCG | GAGAAAGAG | ACTTCGT | CTG | ACGGCCCGGAC | GACAGAACC |
| | (Reverse | | | CTG | ATGCACATC | AGCGG | AACGG | | CTGC | AGACTGTCTT | GTGGTG | CCACCATCA | GCCCGT | GCT | CTACCAGAAAG | AGCTGTACA |
| | Orienta- | | | TTT | GACGCCACA | AGTTA | CATCTT | | TGCT | GTGCCGCCA | GTGGAT | ACTGCAAGA | GTTTCTG | GGA | CACTACCAGCC | ACGAGCTGA |
| | tion)-GM- | | | CTG | CTGTACACC | CACCC | CGTGAT | | GTG | GCGGCTTCA | CT | GCAGCCAGA | CCCGCC | ACA | TTACGCTCCTC | ACCTGGGGA |
| | CSF-Ra | | | GTG | GAGAGCGAC | GAGCC | CTGCTG | | CGA | CCTTCAACA | | GCCTGCTGT | AAGCCT | TGTG | CTAGAGACTTC | GAAGAGAAG |
| | (SS)- | | | GCC | GTGCACCCT | TATCT | CCTGAC | | GCT | AGAACGCCA | | ACTCCAGCA | ACAACA | GTGT | GCCGCCTACCG | AGTACGACG |
| | aGPC3 | | | GCT | AGCTGTAAA | TCAGC | CTACTG | | GCC | TGAACTGGG | | ACCAGAAGA | ACCCCT | CCTG | GTCC | TGCTGGACA |
| | hPY7 vL | | | GCC | GTGACCGCC | CTGAT | CTTCGC | | CCAT | TCCGACAGG | | ACTACCTGG | GCTCCTA | CTGC | | AGCGGAGAG |
| | | | | ACA | ATGAAGTGC | CGGAG | CCCTAG | | CCTG | CCCCTGGCA | | CCTGGTATC | GACCTC | TGA | | GCAGAGATC |
| | (GGGGS) | | | AGA | TTTCTGCTGG | GCGGT | ATGCAG | | CCTT | AAGGCCTTG | | AGCAAAAGC | CTACAC | GCCT | | CTGAGATGG |
| | 3(SEQ | | | GTG | AACTGCAAG | AGCGG | AGAGC | | TCTG | AATGGGTCG | | CCGGCCAGC | CAGCTC | GGT | | GCGGCAAGC |
| | ID NO: | | | CAC | TGATCAGCC | AGGCG | GGCGG | | CTG | GACGGATCC | | CTCCTAAGCT | CTACAA | CATC | | CCAGACGGA |
| | 223)- | | | AGC | TGGAAAGCG | GAGGA | AGAAA | | ATCC | GGAACAAGA | | GCTGATCTAT | TCGCCA | ACC | | AGAATCCTC |
| | aGPC3 | | | | GCGACGCCA | AGTGG | CGAAC | | CT | CCAACAACT | | TGGGCCAGC | GCCAGC | CTGT | | AAGAGGGCC |
| | hPY7 vH | | | | GCATCCACG | TGGCG | GGCTGA | | | ACGCCACCT | | TCCAGAGAA | CTCTGTC | ACT | | TGTATAATG |
| | -CD8FA | | | | ACACCGTGG | GATCT | GAAGA | | | ACTACGCCG | | AGCGGCGTG | TCTGAG | GCA | | AGCTGCAGA |
| | (Hinge)- | | | | AAAACCTGA | CTGCA | GAATCT | | | ACAGCGTGA | | CCCGATAGA | GCCAGA | ACC | | AAGACAAGA |
| | CD8FA | | | | TCATCCTGGC | A | GTGCGG | | | AGGCCAGGT | | TTTTCTGGCT | AGCTTGT | ACC | | TGGCCGAGG |
| | (TM)- | | | | CAACAACAG | | CCCGTT | | | TCACCATCTC | | CTGGCAGCG | AGACCT | GG | | CCTACAGCG |
| | CD28 | | | | CCTGAGCAG | | | | | CAGAGATGA | | GCACCGACT | GCTGCA | | | AGATCGGAA |
| | (ICD)- | | | | CAACGGCAA | | | | | CAGCAAGAA | | TCACCCTGA | GGCGGA | | | TGAAGGGCG |
| | CD3z | | | | TGTGACCGA | | | | | CAGCCTGTA | | CAATTTCTAG | GCCGTG | | | AGCGCAGAA |
| | (ICD) | | | | GTCCGGCTG | | | | | CCTGCAGAT | | CCTGCAAGC | CATACA | | | GAGGCAAGG |
| | | | | | CAAAGAGTG | | | | | GAACTCCCT | | CGAGGACGT | AGAGGA | | | GACACGATG |
| | | | | | CGAGGAACT | | | | | GAAAACCGA | | GGCCGTGTA | CTGGATT | | | GACTGTACC |
| | | | | | GGAAGAGAA | | | | | GGACACCGC | | TTACTGCCA | TCGCCTG | | | AGGGCCTGA |
| | | | | | GAATATCAA | | | | | CGTGTACTAT | | GCAGTACTA | CGAC | | | GCACCGCCA |
| | | | | | AGAGTTCCT | | | | | TGCGTGGCC | | CAACTACCC | | | | CCAAGGATA |
| | | | | | GCAGAGCTT | | | | | GGCAATAGC | | TCTGACCTTC | | | | CCTATGATG |
| | | | | | CGTGCACAT | | | | | TTTGCCTACT | | GGCCAGGGC | | | | CCCTGCACA |
| | | | | | CGTGCAGAT | | | | | GGGGACAGG | | ACCAAGCTG | | | | TGCAGGCCC |
| | | | | | GTTCATCAA | | | | | GCACCCTGG | | GAAATCAAA | | | | TGCCTCCAA |
| | | | | | CACAAGC | | | | | TTACAGTTTC | | | | | | GA |
| | | | | | | | | | | TGCT | | | | | | |
| | SEQ ID | | | 214 | 286 | 288 | 331 | | 217 | 222 | 332 | 333 | 335 | 243 | 268 | 280 |
| | NO | | | | | | | | | | | | | | | |
| | | | AA | MD | NWVNVISDL | SGGGG | LLPSWA | None | MLL | EVQLVESGG | GGGGSG | DIVMTQSPDS | ALSNSIM | IYIW | RSKRSRLLHSD | RVKFSRSADA |
| | | | | WT | KKIEDLIQSM | SGGGG | ITLISVN | | LVTS | GLVQPGGSLR | GGGSGG | LAVSLGERAT | YFSHFVP | APLA | YMNMTPRRPGP | PAYQQGQNQ |
| | | | | WIL | HIDATLYTES | SGVTP | GIFVICC | | LLLC | LSCAASGFTF | GGS | INCKSSQSLL | VFLPAKP | GTCG | TRKHYQPYAP | LYNELNLGRR |
| | | | | FLV | DVHPSCKVT | EPIFSL | LTYCFA | | ELPH | NKNAMNWV | | YSSNQKNYL | TTTPAPR | VLL | PRDFAAYRS | EEYDVLDKR |
| | | | | AAA | AMKCFLLEL | IGGGS | PRCRER | | PAFL | RQAPGKGLE | | AWYQQKPGQ | PPTPAPTI | LSLV | | RGRDPEMGG |
| | | | | TRV | QVISLESGDA | GGGGS | RRNERL | | LIP | WVGRIRNKT | | PPKLLIYWAS | ASQPLSL | ITLY | | KPRRKNPQEG |
| | | | | HS | SIHDTVENLII | GGGSL | RRESVR | | | NNYATYYAD | | SRESGVPDRF | RPEACRP | CNH | | LYNELQKDK |
| | | | | | LANNSLSSNG | Q | PV | | | SVKARFTISR | | SGSGSGTDFT | AAGGAV | | | MAEAYSEIG |
| | | | | | NVTESGCKEC | | | | | DDSKNSLYLQ | | LTISSLQAED | HTRGLDF | | | MKGERRRGK |
| | | | | | EELEEKNIKE | | | | | MNSLKTEDT | | VAVYYCQQY | ACD | | | GHDGLYQGL |
| | | | | | FLQSFVHIVQ | | | | | AVYYCVAGN | | YNYPLTFGQG | | | | STATKDTYD |
| | | | | | MFINTS | | | | | SFAYWGQGT | | TKLEIK | | | | ALHMQALPP |
| | | | | | | | | | | LVTVSA | | | | | | R |
| | | | | | | | | | | | | | | R | | |
| | SEQ ID | | | 218 | 285 | 287 | 219 | | 216 | 206 | 223 | 208 | 353 | 242 | 267 | 279 |
| | NO | | | | | | | | | | | | | | | |
| SB | | | | IgE | IL15 | TaceOP | B7-1 | E2A/T2 | IGM- | hPY7 | (GGGGS) | hPY7 | Hinge | CD8F | 41BB | CD3z |
| 05605 | | | | | | T+LR1 | | A | CSF- | VH | 3(SEQ | VL | CD8a | A | | |
| | | | | | | linker | | | Ra | | ID NO: | | | | | |
| | | | | | | | | | | | 223) | | | | | |
| | (IgE(SS) | | | ATG | AATTGGGTC | TCTGG | CTGCTG | None | ATG | GAAGTGCAG | GGCGG | GACATCGTG | ACCACC | ATCT | AAGCGGGGCA | AGAGTGAAG |
| | -IL15 | | | GAC | AACGTGATC | CGGCG | CCTAGC | | CTGC | CTGGTGGAA | CGGAG | ATGACACAG | ACACCA | ACA | GAAAGAAGCT | TTCAGCAGG |
| | Tace10 | | | TGG | AGCGACCTG | GAGGA | TGGGCC | | TGCT | TCTGGCGGA | GAAGC | AGCCCCGAT | GCTCCTC | TCTG | GCTGTACATCT | AGCGCAGAC |
| | (cleavage | | | ACC | AAGAAGATC | TCTGG | ATCACA | | GGT | GGACTGGTT | GGAGG | AGCCTGGCC | GGCCAC | GGC | TCAAGCAGCCC | GCCCCCGCG |
| | site)-B7- | | | TGG | GAGGACCTG | CGGAG | CTGATC | | CAC | CAACCTGGC | CGGAG | GTGTCTCTGG | CAACTC | CCCT | TTCATGCGGCC | TACAAGCAG |
| | 1(TM)) | | | ATC | ATCCAGAGC | GTGGA | TCCGTG | | ATCT | GGCTCTCTG | GATCCG | GAGAAAGAG | CAGCTC | CTG | CGTGCAGACCA | GGCCAGAAC |
| | (Reverse | | | CTG | ATGCACATC | AGCGG | AACGG | | CTGC | AGACTGTCT | GTGGTG | CCACCATCA | CAACAA | GCT | CACAAGAGGA | CAGCTCTAT |
| | Orienta- | | | TTT | GACGCCACA | AGTTA | CATCTT | | TGCT | TGTGCCGCC | GTGGAT | ACTGCAAGA | TTGCCA | GGA | AGATGGCTGCA | AACGAGCTC |
| | tion)-GM- | | | CTG | CTGTACACC | CACCC | CGTGAT | | GTG | AGCGGCTTC | CT | GCAGCCAGA | GCCAGC | ACA | GCTGTCGGTTC | AATCTAGGA |
| | CSF-Ra | | | GTG | GAGAGCGAC | GAGCC | CTGCTG | | CGA | ACCTTCAACA | | GCCTGCTGT | CTCTGTC | TGTG | CCCGAGGAAG | CGAAGAGAG |
| | (SS)- | | | GCC | GTGCACCCT | TATCT | CCTGAC | | GCT | AGAACGCCA | | ACTCCAGCA | TCTGAG | GTGT | AAGAAGGCGG | GAGTACGAT |
| | aGPC3 | | | GCT | AGCTGTAAA | TCAGC | CTACTG | | GCC | TGAACTGGG | | ACCAGAAGA | GCCCGA | CTTG | CTGCGAGCTG | GTTTTGGAC |
| | hPY7 vL | | | GCC | GTGACCGCC | CTGAT | CTTCGC | | CCAT | TCCGACAGG | | ACTACCTGG | AGCTTGT | CTGC | | AAGAGACGT |
| | (GGGGS) | | | ACA | ATGAAGTGC | CGGAG | CCCTAG | | CCTG | CCCCTGGCA | | CCTGGTATC | AGACCT | TGA | | GGCCGGGAC |
| | 3(SEQ | | | AGA | TTTCTGCTGG | GCGGT | ATGCAG | | CCTT | AAGGCCTTG | | AGCAAAAGC | GCTGCA | GCCT | | CCTGAGATG |
| | ID NO: | | | GTG | AACTGCAAG | AGCGG | AGAGC | | TCTG | AATGGGTCG | | CCGGCCAGC | GGCGGA | GGT | | GGGGGAAAG |
| | 223)- | | | CAC | TGATCAGCC | AGGCG | GGCGG | | CTG | GACGGATCC | | CTCCTAAGCT | GCCGTG | CATC | | CCGAGAAGG |
| | aGPC3 | | | AGC | TGGAAAGCG | GAGGA | AGAAA | | ATCC | GGAACAAGA | | GCTGATCTAT | CATACA | ACC | | AAGAACCCT |
| | hPY7 vH | | | | GCGACGCCA | AGTGG | CGAAC | | CT | CCAACAACT | | TGGGCCAGC | AGAGGA | | | CAGGAAGGC |
| | -CD8a | | | | GCATCCACG | TGGCG | GGCTGA | | | ACGCCACCT | | TCCAGAGAA | CTGGATT | | | CTGTACAAT |
| | (Hinge)- | | | | ACACCGTGG | GATCT | GAAGA | | | ACTACGCCG | | AGCGGCGTG | TCGCCTG | | | GAACTGCAG |
| | CD8 | | | | AAAACCTGA | CTGCA | GAATCT | | | ACAGCGTGA | | CCCGATAGA | CGAC | | | AAAGATAAG |
| | (TM)- | | | | TCATCCTGGC | A | GTGCGG | | | AGGCCAGGT | | TTTTCTGGCT | | | | ATGGCGGAG |
| | 41BB | | | | CAACAACAG | | CCCGTT | | | TCACCATCTC | | CTGGCAGCG | | | | GCCTACAGT |
| | (ICD)- | | | | CCTGAGCAG | | | | | CAGAGATGA | | GCACCGACT | | | | GAGATTGGG |
| | CD3z | | | | CAACGGCAA | | | | | CAGCAAGAA | | TCACCCTGA | | | | ATGAAAGGC |
| | (ICD) | | | | TGTGACCGA | | | | | CAGCCTGTA | | CAATTTCTAG | | | | GAGCGCCGG |
| | | | | | GTCCGGCTG | | | | | CCTGCAGAT | | CCTGCAAGC | | | | AGGGGCAAG |
| | | | | | CAAAGAGTG | | | | | GAACTCCCT | | CGAGGACGT | | | | GGGCACGAT |
| | | | | | CGAGGAACT | | | | | GAAAACCGA | | GGCCGTGTA | | | | GGCCTTTACC |
| | | | | | GGAAGAGAA | | | | | GGACACCGC | | TTACTGCCA | | | | AGGGTCTCA |
| | | | | | GAATATCAA | | | | | CGTGTACTAT | | GCAGTACTA | | | | GTACAGCCA |
| | | | | | AGAGTTCCT | | | | | TGCGTGGCC | | CAACTACCC | | | | CCAAGGACA |
| | | | | | GCAGAGCTT | | | | | GGCAATAGC | | TCTGACCTTC | | | | CCTACGACG |
| | | | | | CGTGCACAT | | | | | TTTGCCTACT | | GGCCAGGGC | | | | CCCTTCACAT |
| | | | | | CGTGCAGAT | | | | | GGGGACAGG | | ACCAAGCTG | | | | GCAGGCCCT |
| | | | | | GTTCATCAA | | | | | GCACCCTGG | | GAAATCAAG | | | | GCCCCCTCG |
| | | | | | CACAAGC | | | | | TTACAGTTTC | | | | | | C |
| | | | | | | | | | | TGCT | | | | | | |
| | SEQ ID | | | 214 | 286 | 288 | 331 | | 217 | 222 | 332 | 336 | 337 | 338 | 339 | 334 |
| | NO | | | | | | | | | | | | | | | |
| | | | | MD | NWVNVISDL | SGGGG | LLPSWA | None | MLL | LSCAASGFTF | GGGGSG | DIVMTQSPDS | TTTPAPR | IYIW | KQPFMRPVQTT | RVKFSRSADA |
| | | | | WT | KKIEDLIQSM | SGGGG | ITLISVN | | LVTS | EVQLVESGG | GGGSGG | LAVSLGERAT | PPTPAPT | APLA | KRGRKKLLYIF | PAYKQGQNQ |
| | | | | WIL | HIDATLYTES | SGVTP | GIFVICC | | LLLC | GLVQPGGSLR | GGS | INCKSSQSLL | IASQPLS | GTC | QEEDGCSCRFP | LYNELNLGRR |
| | | | | FLV | DVHPSCKVT | EPIFSLI | LTYCFA | | ELPH | NKNAMNWV | | YSSNQKNYL | LRPEACR | GVLL | EEEEGGCEL | EEYDVLDKR |
| | | | | AAA | AMKCFLLELQ | GGGSG | PRCRER | | PAFL | RQAPGKGLE | | AWYQQKPGQ | PAAGGAV | LSLV | | RGRDPEMGG |
| | | | | TRV | VISLESGDAS | GGGSG | RRNERL | | LIP | WVGRIRNKT | | PPKLLIYWAS | HTRGLDF | IT | | KPRRKNPQEG |
| | | | | HS | IHDTVENLII | GGSLQ | RRESVR | | | NNYATYYAD | | SRESGVPDRF | ACD | | | LYNELQKDK |
| | | | | | LANNSLSSNG | | PV | | | SVKARFTISR | | SGSGSGTDFT | | | | MAEAYSEIG |
| | | | | | NVTESGCKEC | | | | | DDSKNSLYLQ | | LTISSLQAED | | | | MKGERRRGK |
| | | | | | EELEEKNIKE | | | | | MNSLKTEDT | | VAVYYCQQY | | | | GHDGLYQGL |
| | | | | | FLQSFVHIVQ | | | | | AVYYCVAGN | | YNYPLTFGQG | | | | STATKDTYD |
| | | | | | MFINTS | | | | | SFAYWGQGT | | TKLEIK | | | | ALHMQALPP |
| | | | | | | | | | | LVTVSA | | | | | | R |
| | SEQ ID | | | 218 | 285 | 287 | 219 | | 216 | 206 | 223 | 208 | 196 | 236 | 271 | 277 |
| | NO | | | | | | | | | | | | | | | |

TABLE 22

| SB | Descrip- | Back- | Seq | TM | Cleavage | | Syn | Insu- | | | | | |
|---|---|---|---|---|---|---|---|---|---|---|---|---|---|
| ID | tion | bone | Type | domain | Site | IL12 | promoter | lator | Promoter | SynTF | | | |
| | | | | | LR1 | | | | | | | | |
| | | | | | split | | | | | | | | |
| | | | | | N term | | | | | | | | |
| | | | | | linker + | | YB TATA | | | | | | |
| SB | | | | | CD16 | | 4X ZF5 | | | SV40 | | | |
| 05042 | | | | B7-1 | TACE | IL12p70 | BD | A2 | SV40 | NLS | miniVPR | NS3 | ZF5-7 DBD |
| | B7-1 | Sin | DNA | CTGC | AGCGG | ATGTGTCACCAGCAGCTGGTCAT | AATTAA | ACAA | GTGTGT | ATG | GACGCCCTGGACG | GAGGATGTCGTGT | ATGTCTAGACCT |
| | (TM)- | Vec | | TGCC | CGGAG | CAGCTGGTTCAGCCTGGTGTTCC | cgggtttcgt | TGGC | CAGTTA | CCC | ACTTCGATCTGGA | GCTGCCACAGCAT | GGCGAGAGGCC |
| | CD16 | | | AAG | GTGGTA | TGGCCTCTCCTCTGGTGGCCATC | aacaatcgc | TGGC | GGGTGT | AAG | TATGCTGGGCAGC | CTACGGCAAGAAG | CTTCCAGTGCCG |
| | TACE | | | CTG | GCGGA | TGGGAGCTGAAGAAAGACGTGT | atgaggattc | CCAT | GGAAA | AAA | GACGCTCTGGATG | AAGGGCGACATCG | GATCTGCATGCG |
| | (cleavage | | | GGC | GGCGG | ACGTGGTGGAACTGGACTGGTA | gcaacgect | AGTA | GTCCCC | AAG | ATTTTGACCTGGA | ACACCTACCGGTA | GAACTTCAGCA |
| | site)- | | | CATC | AGGATC | TCCCGATGCTCCTGGCGAGATGG | ttGAAGC | AATG | AGGCTC | CGG | CATGCTCGGCTCT | CATCGGCAGCTCT | ACATGAGCAAC |
| | IL12- | | | ACA | TGGAAT | TGGTGCTGACCTGCGATACCCCT | AGTCGA | CCGT | CCCAGC | AAG | GATGCACTCGACG | GGCACAGGCTGTG | CTGACCAGACA |
| | YB TAT | | | CTG | TACACA | GAAGAGGACGGCATCACCTGGA | CGCCGA | GTTA | AGGCA | GTG | ATTTCGACCTCGA | TGGTCATCGTGGG | CACCCGGACAC |
| | A ZFBD | | | ATCT | GGGACT | CACTGGATCAGTCTAGCGAGGT | Agtcccgtc | GTGT | GAAGT | | TATGTTGGGATCT | CAGAATCGTGCTG | ACACAGGCGAG |
| | (syn | | | CCGT | CGCCGT | GCTCGGCAGCGGCAAGACCCTG | tcagtaaag | GTTA | ATGCAA | | GATGCCCTTGATG | TCTGGCAGCGGAA | AAGCCTTTTCAG |
| | prmoter)- | | | GAA | GTCTAC | ACCATCCAAGTGAAAGAGTTTG | gttGAAG | GTTG | AGCATG | | ACTTTGATCTCGA | CAAGCGCCCCTAT | TGCAGAATCTGT |
| | A2 (insu- | | | CGG | AATCTC | GCGACGCCGGCCAGTACACCTG | CAGTCG | CTGT | CATCTC | | CATGTTGATCAAT | CACAGCCTATGCT | ATGCGCAATTTC |
| | lator)- | | | CATC | CAGCTT | TCACAAAGGCGGAGAAGTGCTG | ACGCCG | TCTTC | AATTAG | | AGCCGGTCCAGCG | CAGCAGACAAGA | TCCGACAGAAG |
| | SV40 | | | TTCG | CTTTGG | AGCCACAGCCTGCTGCTGCTCCA | AAgaatcg | CACG | TCAGCA | | GCAGCCCCAAGAA | GGCCTGCTGGGCT | CGTGCTGCGGA |
| | (promoter)- | | | TGAT | TGGCGG | CAAGAAAGAGGATGGCATTTGG | gactgccttc | TCAG | ACCAG | | GAAGAGAAAAGT | GCATCATCACAAG | GACACCTGAGA |
| | Syn TF | | | CTGT | TAGTGG | AGCACCGACATCCTGAAGGACC | gtatGAA | AAGA | GTGTGG | | CGGCTCTGGCGGC | CCTGACCGGCAGA | ACCCACACCGG |
| | (NLS + | | | TGCC | CGGCG | AGAAAGAGCCCAAGAACAAGAC | GCAGTC | GGCA | AAAGTC | | GGATCTGGCGGTT | GACAAGAACCAG | CAGCCAGAAAC |
| | miniVPR | | | TGA | GTGGCA | CTTCCTGAGATGCGAGGCCAAG | GACGCC | CAGA | CCCAGG | | CTGGATCTGTTTT | GTGGAAGGCGAG | CATTCCAGTGTC |
| | activation | | | CCTA | GTGGCG | AACTACAGCGGCCGGTTCACAT | GAAggtat | CAAA | CTCCCC | | GCCCCAAGCTCCT | GTGCAGATCGTGT | GCATCTGTATGA |
| | domain + | | | CTGC | GTGGAT | GTTGGTGGCTGACCACCATCAGC | cagtegcct | TTAC | AGCAG | | GCTCCTGCACCAG | CTACAGCTACCCA | GAAACTTTAGC |
| | NS3 | | | TTCG | CTCTTC | ACCGACCTGACCTTCAGCGTGA | cggaatGA | CACC | GCAGA | | CTCCAGCTATGGT | GACCTTCCTGGCC | GACCCCTCCAAT |
| | protease + | | | CCCC | AA | AGTCCAGCAGAGGCAGCAGTGA | AGCAGT | AGGT | AGTATG | | TTCTGCTCTGGCTC | ACCTGTATCAATG | CTGGCCCGGCA |
| | ZFBD | | | TCG | | TCCTCAGGGCGTTACATGTGGCG | CGACGC | GGCG | CAAAG | | AGGCTCCAGCTCC | GCGTGTGCTGGGC | CACCAGAACAC |
| | DNA | | | GTG | | CCGCTACACTGTCTGCCGAAAG | CGAAgat | CTCA | CATGCA | | TGTGCCTGTTCTTG | CGTGTATCACGGC | ATACCGGGGAA |
| | binding | | | CAG | | AGTGCGGGGCGACAACAAAGAA | tcgtaagag | GAGT | TCTCAA | | CTCCTGGACCTCC | GCTGGAACCAGAA | AAACCCTTTCAG |
| | domain) | | | AGA | | TACGAGTACAGCGTGGAATGCC | gctcactctc | CTGC | TTAGTC | | TCAGGCTGTTGCT | CAATCGCCTCTCC | TGTAGGATATGC |
| | | | | GCG | | AAGAGGACAGCGCCTGTCCAGC | ccttacacg | GGAG | AGCAA | | CCACCAGCACCTA | TAAGGGCCCCGTG | ATGAGGAATTTT |
| | | | | GAG | | CGCCGAAGAGTCTCTGCCTATCG | gagtggata | GCAT | CCATAG | | AACCTACACAGGC | ATCCAGATGTACA | TCCGACCGGTCC |
| | | | | AAG | | AAGTGATGGTGGACGCCGTGCA | ACTAGT | CACA | TCCCGC | | CGGCGAGGGAAC | CCAACGTGGACCA | AGCCTGAGGCG |
| | | | | AAA | | CAAGCTGAAGTACGAGAACTAC | TCTAGA | ACAG | CCCTAA | | ACTGTCTGAAGCT | GGACCTCGTTGGC | GCACCTGAGGA |
| | | | | CGA | | ACCTCCAGCTTTTTCATCCGGGA | GGGTAT | CCCT | CTCCGC | | CTGCTGCAGCTCC | TGGCCTGCTCCTC | CACATACTGGCT |
| | | | | ACG | | CATCATCAAGCCCGATCCTCCAA | ATAATG | GAAT | CCATCC | | AGTTCGACGACGA | AAGGCAGCAGAA | CCCAAAAGCCG |
| | | | | GCT | | AGAACCTGCAGCTGAAGCCTCT | GGGGC | TTGA | CGCCCC | | AGATCTGGGAGCC | GCCTGACACCTTG | TTCCAATGTCGG |
| | | | | GCG | | GAAGAACAGCAGACAGGTGGAA | CAACGC | ATCC | TAATTT | | CTGCTGGGCAATA | CACCTGTGGCTCC | ATATGTATGCGC |
| | | | | GAG | | GTGTCCTGGGAGTACCCCGACA | GTACCG | TGCT | TAACTC | | GCACAGATCCTGC | AGCGATCTGTACC | AACTTTAGCCAG |
| | | | | AGA | | CCTGGTCTACACCCCACAGCTAC | GTGTC | CTGC | CGCCCA | | CGTGTTCACCGAT | TGGTCACCAGACA | AGCGGCACCCT |
| | | | | ATCT | | TTCAGCCTGACCTTTTGCGTGCA | | CACT | GTTCCG | | CTGGCCAGCGTGG | CGCCGACGTGATC | GCACAGACACA |
| | | | | GTG | | AGTGCAGGGCAAGTCCAAGCGC | | GCCT | CCCATT | | ACAATAGCGAGTT | CCTGTCAGAAGAA | CAAGAACCCAT |
| | | | | CGG | | GAGAAAAAGGACCGGGTGTTCA | | AGTT | CTCCGC | | CCAGCAGCTCCTG | GAGGGGATTCCAG | ACTGGCGAGAA |
| | | | | CCTG | | CCGACAAGACCAGCGCCACCGT | | GAGA | CCCATG | | AACCAGGGCATTC | AGGCAGCCTGCTG | ACCTTTCCAATG |
| | | | | TG | | GATCTGCAGAAAGAACGCCAGC | | CCTTT | GCTGAC | | CTGTGGCTCCTCA | AGCCCTAGACCTA | TAGAATCTGCAT |
| | | | | | | ATCAGCGTCAGAGCCCAGGACC | | TACT | TTTTTA | | CACCACCGAGCCT | TCAGCTACCTGAA | GCGAAATTTTTC |
| | | | | | | GGTACTACAGCAGCTCTTGGAG | | ACCT | TTTATG | | ATGCTGATGGAAT | GGGCTCTAGCGGC | CCAGCGGCCTA |
| | | | | | | CGAATGGGCCAGCGTGCCATGT | | GACT | CAGAG | | ACCCCGAGGCCAT | GGACCTCTGCTTT | ATCTGACCAGG |
| | | | | | | TCTGGCGGAGGAAGCGGTGGCG | | AGCT | GCCGA | | CACCAGACTGGTC | GTCCTGCTGGACA | CATCTGAGGAC |
| | | | | | | GATCAGGTGGTGGATCTGGCGG | | GAGA | GGCCGC | | ACCGGTGCTCAAA | TGCCGTGGGCCTG | CCACCTGAGAG |
| | | | | | | CGGATCTAGAAACCTGCCTGTG | | CATT | CTCTGC | | GACCACCTGATCC | TTTAGAGCCGCCG | GATCT |
| | | | | | | GCCACTCCTGATCCTGGCATGTT | | TACG | CTCTGA | | GGCTCCAGCACCT | TGTGTACAAGAGG | |
| | | | | | | CCCTTGTCTGCACCACAGCCAGA | | ACAT | GCTATT | | CTTGGAGCACCTG | CGTGGCCAAAGCC | |
| | | | | | | ACCTGCTGAGAGCCGTGTCCAA | | TTAC | CCAGA | | GACTGCCTAATGG | GTGGACTTCATCC | |
| | | | | | | CATGCTGCAGAAGGCCAGACAG | | TGGC | AGTAGT | | ACTGCTGTCTGGC | CCGTGGAAAACCT | |
| | | | | | | ACCCTGGAATTCTACCCCTGCAC | | TCTA | GAGGA | | GACGAGGACTTCA | GGAAACCACCATG | |
| | | | | | | CAGCGAGGAAATCGACCACGAG | | GGAC | GGCTTT | | GCTCTATCGCCGA | CGGAGCCCCGTGT | |
| | | | | | | GACATCACCAAGGATAAGACCA | | TCAT | TTTGGA | | CATGGATTTCAGC | TCACCGACAATTC | |
| | | | | | | GCACCGTGGAAGCCTGCCTGCCT | | TTTAT | GGCCTA | | GCCCTGCTCAGTG | TAGCCCTCCAGCC | |
| | | | | | | CTGGAACTGACCAAGAACGAGA | | TCAT | GGCTTT | | GCGGTGGAAGCGG | GTGACACTGACAC | |
| | | | | | | GCTGCCTGAACAGCCGGGAAAC | | TTCA | TGCAAA | | AGGAAGTGGCAGC | ACCCCATCACCAA | |
| | | | | | | CAGCTTCATCACCAACGGCTCTT | | TTAC | | | GATCTTTCTCACC | GATCGACAGAGAG | |
| | | | | | | GCCTGGCCAGCAGAAAGACCTC | | TTTTT | | | CTCCACCTAGAGG | GTGCTGTACCAAG | |
| | | | | | | CTTCATGATGGCCCTGTGCCTGA | | TTTTC | | | CCACCTGGACGAG | AGTTCGACGAGAT | |
| | | | | | | GCAGCATCTACGAGGACCTGAA | | TTTG | | | CTGACAACCACAC | GGAAGAGTGCAGC | |
| | | | | | | GATGTACCAGGTGGAATTCAAG | | AGAC | | | TGGAATCCATGAC | CAGCAC | |
| | | | | | | ACCATGAACGCCAAGCTGCTGA | | GGAA | | | CGAGGACCTGAAC | | |
| | | | | | | TGGACCCCAAGCGGCAGATCTT | | TCTC | | | CTGGACAGCCCTC | | |
| | | | | | | CCTGGACCAGAATATGCTGGCC | | GCTC | | | TGACACCCGAGCT | | |
| | | | | | | GTGATCGACGAGCTGATGCAGG | | T | | | GAACGAGATCCTG | | |
| | | | | | | CCCTGAACTTCAACAGCGAGAC | | | | | GACACCTTCCTGA | | |
| | | | | | | AGTGCCCCAGAAGTCTAGCCTG | | | | | ACGACGAGTGTCT | | |
| | | | | | | GAAGAACCCGACTTCTACAAGA | | | | | GCTGCACGCCATG | | |
| | | | | | | CCAAGATCAAGCTGTGCATCCTG | | | | | CACATCTCTACCG | | |
| | | | | | | CTGCACGCCTTCCGGATCAGAGC | | | | | GCCTGAGCATCTT | | |
| | | | | | | CGTGACCATCGACAGAGTGATG | | | | | CGACACCAGCCTG | | |
| | | | | | | AGCTACCTGAACGCCTCT | | | | | TTT | | |
| | SEQ ID | | | 220 | 291 | 294 | 298 | 300 | 295 | 340 | 322 | 195 | 323 |
| | NO | | | | | | | | | | | | |
| | | | AA | LLPS | SGGGGS | MCHQQLVISWFSLVFLASPLVAIW | | | | MPK | DALDDFDLDMLGS | EDVVCCHSIYGKK | MSRPGERPFQCRI |
| | | | | WAIT | GGGGSG | ELKKDVYVVELDWYPDAPGEMV | | | | KKR | DALDDFDLDMLGS | KGDIDTYRYIGSSG | CMRNFSNMSNLT |
| | | | | LISV | ITQGLA | VLTCDTPEEDGITWTLDQSSEVLG | | | | KV | DALDDFDLDMLGS | TGCVVIVGRIVLSG | RHTRTHTGEKPF |
| | | | | NGIF | VSTISSF | SGKTLTIQVKEFGDAGQYTCHKG | | | | | DALDDFDLDMLIN | SGTSAPITAYAQQT | QCRICMRNFSDR |
| | | | | VICC | FGGGSG | GEVLSHSLLLLHKKEDGIWSTDIL | | | | | SRSSGSPKKKRKVG | RGLLGCIITSLTGRD | SVLRRHLRTHTG |
| | | | | LTYC | GSLQ | KDQKEPKNKTFLRCEAKNYSGRF | | | | | SGGGSGGSGSVLPQ | KNQVEGEVQIVST | SQKPFQCRICMR |
| | | | | FAPR | | TCWWLTTISTDLTFSVKSSRGSSD | | | | | APAPAPAPAMVSA | ATQTFLATCINGVC | NFSDPSNLARHT |
| | | | | CRER | | PQGVTCGAATLSAERVRGDNKEY | | | | | LAQAPAPVPVLAP | WAVYHGAGTRTIA | CMRNFSDRSSLR |
| | | | | RRNE | | EYSVECQEDSACPAAEESLPIEVM | | | | | GPPQAVAPPAPKPT | SPKGPVIQMYTNV | RHLRTHTGSQKP |
| | | | | RLRR | | VDAVHKLKYENYTSSFFIRDIIKPD | | | | | QAGEGTLSEALLQL | DQDLVGWPAPQGS | FQCRICMRNFSQ |
| | | | | ESVR | | PPKNLQLKPLKNSRQVEVSWEYP | | | | | QFDDEDLGALLGN | RSLTPCTCGSSDLY | SGTLHRHTRTHT |
| | | | | PV | | DTWSTPHSYFSLTFCVQVQGKSK | | | | | STDPAVFTDLASVD | LVTRHADVIPVRRR | GEKPFQCRICMR |
| | | | | | | REKKDRVFTDKTSATVICRKNASI | | | | | NSEFQQLLNQGIPV | GDSRGSLLSPRPISY | NFSQRPNLTRHL |
| | | | | | | SVRAQDRYYSSSWSEWASVPCSG | | | | | APHTTEPMLMEYP | LKGSSGGPLLCPAG | RTHLRGS |
| | | | | | | GGSGGGSGGGSGGGSRNLPVATP | | | | | EAITRLVTGAQRPP | HAVGLFRAAVCTR | |
| | | | | | | DPGMFPCLHHSQNLLRAVSNMLQ | | | | | DPAPAPLGAPGLPN | GVAKAVDFIPVENL | |
| | | | | | | KARQTLEFYPCTSEEIDHEDITKD | | | | | GLLSGDEDFSSIAD | ETTMRSPVFTDNSS | |
| | | | | | | KTSTVEACLPLELTKNESCLNSRE | | | | | MDFSALLSGGGSG | PPAVTLTHPITKIDR | |
| | | | | | | TSFITNGSCLASRKTSFMMALCLS | | | | | GSGSDLSHPPPRGH | EVLYQEFDEMEEC | |
| | | | | | | SIYEDLKMYQVEFKTMNAKLLM | | | | | LDELTTTLESMTED | SQH | |
| | | | | | | DPKRQIFLDQNMLAVIDELMQAL | | | | | LNLDSPLTPELNEI | | |
| | | | | | | NFNSETVPQKSSLEEPDFYKTKIK | | | | | LDTFLNDECLLHAM | | |
| | | | | | | LCILLHAFRIRAVTIDRVMSYLNAS | | | | | HISTGLSIFDTSLF | | |
| | SEQ ID | | | 219 | 290 | 293 | | | | 296 | 325 | 321 | 320 |
| | NO | | | | | | | | | | | | |
| | | | | | LR1 | | | | | | | | |
| | | | | | split | | | | | | | | |
| | | | | | N term | | | | | | | | |
| | | | | | linker + | | YB_TATA | | | | | | |
| SB | | | | | CD16 | | 4X ZF5 | | | SV40 | | | |
| 05058 | | | | B7-1 | TACE | IL12p70 | BD | A2 | SV40 | NLS | ZF5-7 DBD | NS3 | mini VPR |
| | B7-1 | Sin | DNA | CTGC | AGCGG | ATGTGTCACCAGCAGCTGGTCAT | AATTAA | ACAA | GTGTGT | ATG | ATGTCTAGACCTG | GAGGATGTCGTGT | GACGCCCTGGA |
| | (TM)- | Vec | | TGCC | CGGAG | CAGCTGGTTCAGCCTGGTGTTCC | cgggtttcgt | TGGC | CAGTTA | CCC | GCGAGAGGCCCTT | GCTGCCACAGCAT | CGACTTCGATCT |
| | CD16 | | | AAG | GTGGTA | TGGCCTCTCCTCTGGTGGCCATC | aacaatcgc | TGGC | GGGTGT | AAG | CCAGTGCCGGATC | CTACGGCAAGAAG | GGATATGCTGG |
| | TACE | | | CTG | GCGGA | TGGGAGCTGAAGAAAGACGTGT | atgaggattc | CCAT | GGAAA | AAA | TGCATGCGGAACT | AAGGGCGACATCG | GCAGCGACGCT |
| | (cleavage | | | GGC | GGCGG | ACGTGGTGGAACTGGACTGGTA | gcaacgcct | AGTA | GTCCCC | AAG | TCAGCAACATGAG | ACACCTACCGGTA | CTGGATGATTTT |
| | site)- | | | CATC | AGGATC | TCCCGATGCTCCTGGCGAGATGG | ttGAAGC | AATG | AGGCTC | CGG | CAACCTGACCAGA | CATCGGCAGCTCT | GACCTGGACAT |
| | IL12 | | | ACA | TGGAAT | TGGTGCTGACCTGCGATACCCCT | AGTCGA | CCGT | CCCAGC | AAG | CACACCCGGACAC | GGCACAGGCTGTG | GCTCGGCTCTGA |
| | YB_TATA | | | CTG | TACACA | GAAGAGGACGGCATCACCTGGA | CGCCGA | GTTA | AGGCA | GTG | ACACAGGCGAGA | TGGTCATCGTGGG | TGCACTCGACG |
| | ZFBD | | | ATCT | GGGACT | CACTGGATCAGTCTAGCGAGGT | Agtcccgtc | GTGT | GAAGT | | AGCCTTTTCAGTG | CAGAATCGTGCTG | ATTTCGACCTCG |
| | (syn | | | CCGT | CGCCGT | GCTCGGCAGCGGCAAGACCCTG | tcagtaaag | GTTA | ATGCAA | | CAGAATCTGTATG | TCTGGCAGCGGAA | ATATGTTGGGAT |
| | prmoter)- | | | GAA | GTCTAC | ACCATCCAAGTGAAAGAGTTTG | gttGAAG | GTTG | AGCATG | | CGCAATTTCTCCG | CAAGCGCCCCTAT | CTGATGCCCTTG |
| | A2 (insu- | | | CGG | AATCTC | GCGACGCCGGCCAGTACACCTG | CAGTCG | CTGT | CATCTC | | ACAGAAGCGTGCT | CACAGCCTATGCT | ATGACTTTGATC |
| | lator)- | | | CATC | CAGCTT | TCACAAAGGCGGAGAAGTGCTG | ACGCCG | TCTTC | AATTAG | | GCGGAGACACCTG | CAGCAGACAAGA | TCGACATGTTGA |
| | SV40 | | | TTCG | CTTTGG | AGCCACAGCCTGCTGCTGCTCCA | AAgaatcg | CACG | TCAGCA | | AGAACCCACACCG | GGCCTGCTGGGCT | TCAATAGCCGGT |
| | (promoter)- | | | TGAT | TGGCGG | CAAGAAAGAGGATGGCATTTGG | gactgccttc | TCAG | ACCAG | | GCAGCCAGAAACC | GCATCATCACAAG | CCAGCGGCAGC |
| | Syn TF | | | CTGT | TAGTGG | AGCACCGACATCCTGAAGGACC | gtatGAA | AAGA | GTGTGG | | ATTCCAGTGTCGC | CCTGACCGGCAGA | CCCAAGAAGAA |
| | (NLS + | | | TGCC | CGGCG | AGAAAGAGCCCAAGAACAAGAC | GCAGTC | GGCA | AAAGTC | | ATCTGTATGAGAA | GACAAGAACCAG | GAGAAAAGTCG |
| | ZFBD | | | TGA | GTGGCA | CTTCCTGAGATGCGAGGCCAAG | GACGCC | CAGA | CCCAGG | | ACTTTAGCGACCC | GTGGAAGGCGAG | GCTCTGGCGGC |
| | DNA | | | CCTA | GTGGCG | AACTACAGCGGCCGGTTCACAT | GAAggtat | CAAA | CTCCCC | | CTCCAATCTGGCC | GTGCAGATCGTGT | GGATCTGGCGG |
| | binding | | | CTGC | GTGGAT | GTTGGTGGCTGACCACCATCAGC | cagtcgcct | TTAC | AGCAG | | CGGCACACCAGAA | CTACAGCTACCCA | TTCTGGATCTGT |
| | domain + | | | TTCG | CTCTTC | ACCGACCTGACCTTCAGCGTGA | cggaatGA | CACC | GCAGA | | CACATACCGGGGA | GACCTTCCTGGCC | TTTGCCCCAAGC |
| | NS3 | | | CCCC | AA | AGTCCAGCAGAGGCAGCAGTGA | AGCAGT | AGGT | AGTATG | | AAAACCCTTTCAG | ACCTGTATCAATG | TCCTGCTCCTGC |
| | protease + | | | TCG | | TCCTCAGGGCGTTACATGTGGCG | CGACGC | GGCG | CAAAG | | TGTAGGATATGCA | GCGTGTGCTGGGC | ACCAGCTCCAG |
| | miniVPR | | | GTG | | CCGCTACACTGTCTGCCGAAAG | CGAAgat | CTCA | CATGCA | | TGAGGAATTTTTC | CGTGTATCACGGC | CTCTGGCTCAGG |
| | activation | | | CAG | | AGTGCGGGGCGACAACAAAGAA | tcgtaagag | GAGT | TCTCAA | | CGACCGGTCCAGC | GCTGGAACCAGAA | CTATGGTTTCTG |
| | domain) | | | AGA | | TACGAGTACAGCGTGGAATGCC | gctcactctc | CTGC | TTAGTC | | CTGAGGCGGCACC | CAATCGCCTCTCC | CTCCAGCTCCTG |
| | | | | GCG | | AAGAGGACAGCGCCTGTCCAGC | ccttacacg | GGAG | AGCAA | | TGAGGACACATAC | TAAGGGCCCCGTG | TGCCTGTTCTTG |
| | | | | GAG | | CGCCGAAGAGTCTCTGCCTATCG | gagtggata | GCAT | CCATAG | | TGGCTCCCAAAAG | ATCCAGATGTACA | CTCCTGGACCTC |
| | | | | AAG | | AAGTGATGGTGGACGCCGTGCA | ACTAGT | CACA | TCCCGC | | CCGTTCCAATGTC | CCAACGTGGACCA | CTCAGGCTGTTG |
| | | | | AAA | | CAAGCTGAAGTACGAGAACTAC | TCTAGA | TGCT | CCCTAA | | GGATATGTATGCG | GGACCTCGTTGGC | CTCCACCAGCAC |
| | | | | CGA | | ACCTCCAGCTTTTTCATCCGGGA | GGGTAT | ACAG | CTCCGC | | CAACTTTAGCCAG | TGGCCTGCTCCTC | CTAAACCTACAC |
| | | | | ACG | | CATCATCAAGCCCGATCCTCCAA | ATAATG | CCCT | CCATCC | | AGCGGCACCCTGC | AAGGCAGCAGAA | AGGCCGGCGAG |
| | | | | GCT | | AGAACCTGCAGCTGAAGCCTCT | GGGGC | GAAT | CGCCCC | | ACAGACACACAAG | GCCTGACACCTTG | GGAACACTGTCT |
| | | | | GCG | | GAAGAACAGCAGACAGGTGGAA | CAACGC | TTGA | TAACTC | | AACCCATACTGGC | CACCTGTGGCTCC | GAAGCTCTGCTG |
| | | | | GAG | | GTGTCCTGGGAGTACCCCGACA | GTACCG | ATCC | CGCCCA | | GAGAAACCTTTCC | AGCGATCTGTACC | CAGCTCCAGTTC |
| | | | | AGA | | CCTGGTCTACACCCCACAGCTAC | GTGTC | CTGC | GTTCCG | | AATGTAGAATCTG | TGGTCACCAGACA | GACGACGAAGA |
| | | | | ATCT | | TTCAGCCTGACCTTTTGCGTGCA | | CACT | CCCATT | | CATGCGAAATTTT | CGCCGACGTGATC | TCTGGGAGCCCT |
| | | | | GTG | | AGTGCAGGGCAAGTCCAAGCGC | | GCCT | CTCCGC | | TCCCAGCGGCCTA | CCTGTCAGAAGAA | GCTGGGCAATA |
| | | | | CGG | | GAGAAAAAGGACCGGGTGTTCA | | AGTT | CCCATG | | ATCTGACCAGGCA | GAGGGGATTCCAG | GCACAGATCCT |
| | | | | CCTG | | CCGACAAGACCAGCGCCACCGT | | GAGA | GCTGAC | | TCTGAGGACCCAC | AGGCAGCCTGCTG | GCCGTGTTCACC |
| | | | | TG | | GATCTGCAGAAAGAACGCCAGC | | CCTTT | TAATTT | | CTGAGAGGATCT | AGCCCTAGACCTA | GATCTGGCCAG |
| | | | | | | ATCAGCGTCAGAGCCCAGGACC | | TACT | TTTTTA | | | TCAGCTACCTGAA | CGTGGACAATA |
| | | | | | | GGTACTACAGCAGCTCTTGGAG | | ACCT | TTTATG | | | GGGCTCTAGCGGC | GCGAGTTCCAG |
| | | | | | | CGAATGGGCCAGCGTGCCATGT | | GACT | CAGAG | | | GGACCTCTGCTTT | CAGCTCCTGAAC |
| | | | | | | TCTGGCGGAGGAAGCGGTGGCG | | AGCT | GCCGA | | | GTCCTGCTGGACA | CAGGGCATTCCT |
| | | | | | | GATCAGGTGGTGGATCTGGCGG | | GAGA | GGCCGC | | | TGCCGTGGGCCTG | GTGGCTCCTCAC |
| | | | | | | CGGATCTAGAAACCTGCCTGTG | | CATT | CTCTGC | | | TTTAGAGCCGCCG | ACCACCGAGCC |
| | | | | | | GCCACTCCTGATCCTGGCATGTT | | TACG | CTCTGA | | | TGTGTACAAGAGG | TATGCTGATGGA |
| | | | | | | CCCTTGTCTGCACCACAGCCAGA | | ACAT | GCTATT | | | CGTGGCCAAAGCC | ATACCCCGAGG |
| | | | | | | ACCTGCTGAGAGCCGTGTCCAA | | TTAC | CCAGA | | | GTGGACTTCATCC | CCATCACCAGA |
| | | | | | | CATGCTGCAGAAGGCCAGACAG | | TGGC | AGTAGT | | | CCGTGGAAAACCT | CTGGTCACCGGT |
| | | | | | | ACCCTGGAATTCTACCCCTGCAC | | TCTA | GAGGA | | | GGAAACCACCATG | GCTCAAAGACC |
| | | | | | | CAGCGAGGAAATCGACCACGAG | | GGAC | GGCTTT | | | CGGAGCCCCGTGT | ACCTGATCCGGC |
| | | | | | | GACATCACCAAGGATAAGACCA | | TCAT | TTTGGA | | | TCACCGACAATTC | TCCAGCACCTCT |
| | | | | | | GCACCGTGGAAGCCTGCCTGCCT | | TTTAT | GGCCTA | | | TAGCCCTCCAGCC | TGGAGCACCTG |
| | | | | | | CTGGAACTGACCAAGAACGAGA | | TCAT | GGCTTT | | | GTGACACTGACAC | GACTGCCTAATG |
| | | | | | | GCTGCCTGAACAGCCGGGAAAC | | TTCA | TGCAAA | | | ACCCCATCACCAA | TTTCAGCGCCCT |
| | | | | | | CAGCTTCATCACCAACGGCTCTT | | TTAC | | | | GATCGACAGAGAG | GACTGCTGTCTG |
| | | | | | | GCCTGGCCAGCAGAAAGACCTC | | TTTTT | | | | GTGCTGTACCAAG | GCGACGAGGAC |
| | | | | | | CTTCATGATGGCCCTGTGCCTGA | | TTTTC | | | | AGTTCGACGAGAT | TTCAGCTCTATC |
| | | | | | | GCAGCATCTACGAGGACCTGAA | | TTTG | | | | GGAAGAGTGCAGC | GCCGACATGGA |
| | | | | | | GATGTACCAGGTGGAATTCAAG | | AGAC | | | | CAGCAC | TTTCAGCGCCCT |
| | | | | | | ACCATGAACGCCAAGCTGCTGA | | GGAA | | | | | GCTCAGTGGCG |
| | | | | | | TGGACCCCAAGCGGCAGATCTT | | TCTC | | | | | GTGGAAGCGGA |
| | | | | | | CCTGGACCAGAATATGCTGGCC | | GCTC | | | | | GGAAGTGGCAG |
| | | | | | | GTGATCGACGAGCTGATGCAGG | | T | | | | | CGATCTTTCTCA |
| | | | | | | CCCTGAACTTCAACAGCGAGAC | | | | | | | CCCTCCACCTAG |
| | | | | | | AGTGCCCCAGAAGTCTAGCCTG | | | | | | | AGGCCACCTGG |
| | | | | | | GAAGAACCCGACTTCTACAAGA | | | | | | | ACGAGCTGACA |
| | | | | | | CCAAGATCAAGCTGTGCATCCTG | | | | | | | ACCACACTGGA |
| | | | | | | CTGCACGCCTTCCGGATCAGAGC | | | | | | | ATCCATGACCG |
| | | | | | | CGTGACCATCGACAGAGTGATG | | | | | | | AGGACCTGAAC |
| | | | | | | AGCTACCTGAACGCCTCT | | | | | | | CTGGACAGCCCT |
| | | | | | | | | | | | | | CTGACACCCGA |
| | | | | | | | | | | | | | GCTGAACGAGA |
| | | | | | | | | | | | | | TCCTGGACACCT |
| | | | | | | | | | | | | | TCCTGAACGAC |
| | | | | | | | | | | | | | GAGTGTCTGCTG |
| | | | | | | | | | | | | | CACGCCATGCA |
| | | | | | | | | | | | | | CATCTCTACCGG |
| | | | | | | | | | | | | | CCTGAGCATCTT |
| | | | | | | | | | | | | | CGACACCAGCC |
| | | | | | | | | | | | | | TGTTT |
| | SEQ ID | | | 220 | 291 | 294 | 298 | 300 | 295 | 340 | 323 | 195 | 322 |
| | NO | | | | | | | | | | | | |
| | | | AA | LLPS | SGGGGS | MCHQQLVISWFSLVFLASPLVAIW | | | | MPK | MRNFSNMSNLTRH | EDVVCCHSIYGKK | DALDDFDLDML |
| | | | | WAIT | GGGGSG | ELKKDVYVVELDWYPDAPGEMV | | | | KKR | MSRPGERPFQCRIC | KGDIDTYRYIGSSG | GSDALDDFDLD |
| | | | | LISV | ITQGLA | VLTCDTPEEDGITWTLDQSSEVLG | | | | KV | TRTHTGEKPFQCRI | TGCVVIVGRIVLSG | MLGSDALDDFDL |
| | | | | NGIF | VSTISSF | SGKTLTIQVKEFGDAGQYTCHKG | | | | | CMRNFSDRSVLRR | SGTSAPITAYAQQT | DMLGSDALDDF |
| | | | | VICC | FGGGSG | GEVLSHSLLLLHKKEDGIWSTDIL | | | | | HLRTHTGSQKPFQC | RGLLGCIITSLTGRD | DLDMLINSRSSG |
| | | | | LTYC | GGGSGG | KDQKEPKNKTFLRCEAKNYSGRF | | | | | RICMRNFSDPSNLA | KNQVEGEVQIVST | SPKKKRKVGSGG |
| | | | | FAPR | GSLQ | TCWWLTTISTDLTFSVKSSRGSSD | | | | | RHTRTHTGEKPFQC | ATQTFLATCINGVC | GSGGSGSVLPQA |
| | | | | CRER | | PQGVTCGAATLSAERVRGDNKEY | | | | | RICMRNFSDRSSLR | WAVYHGAGTRTIA | PAPAPAPAMVSA |
| | | | | RRNE | | EYSVECQEDSACPAAEESLPIEVM | | | | | RHLRTHTGSQKPFQ | SPKGPVIQMYTNV | LAQAPAPVPVLA |
| | | | | RLRR | | VDAVHKLKYENYTSSFFIRDIIKPD | | | | | CRICMRNFSQSGTL | DQDLVGWPAPQGS | PGPPQAVAPPAP |
| | | | | ESVR | | PPKNLQLKPLKNSRQVEVSWEYP | | | | | HRHTRTHTGEKPF | RSLTPCTCGSSDLY | KPTQAGEGTLSE |
| | | | | PV | | DTWSTPHSYFSLTFCVQVQGKSK | | | | | QCRICMRNFSQRPN | LVTRHADVIPVRRR | ALLQLQFDDEDL |
| | | | | | | REKKDRVFTDKTSATVICRKNASI | | | | | LTRHLRTHLRGS | GDSRGSLLSPRPISY | GALLGNSTDPAV |
| | | | | | | SVRAQDRYYSSSWSEWASVPCSG | | | | | | LKGSSGGPLLCPAG | FTDLASVDNSEF |
| | | | | | | GGSGGGSGGGSGGGSRNLPVATP | | | | | | HAVGLFRAAVCTR | QQLLNQGIPVAP |
| | | | | | | DPGMFPCLHHSQNLLRAVSNMLQ | | | | | | GVAKAVDFIPVENL | HTTEPMLMEYPE |
| | | | | | | KARQTLEFYPCTSEEIDHEDITKD | | | | | | ETTMRSPVFTDNSS | AITRLVTGAQRP |
| | | | | | | KTSTVEACLPLELTKNESCLNSRE | | | | | | PPAVTLTHPITKIDR | PDPAPAPLGAPG |
| | | | | | | TSFITNGSCLASRKTSFMMALCLS | | | | | | EVLYQEFDEMEEC | LPNGLLSGDEDF |
| | | | | | | SIYEDLKMYQVEFKTMNAKLLM | | | | | | SQH | SSIADMDFSALLS |
| | | | | | | DPKRQIFLDQNMLAVIDELMQAL | | | | | | | GGGSGGSGSDLS |
| | | | | | | NFNSETVPQKSSLEEPDFYKTKIK | | | | | | | HPPPRGHLDELT |
| | | | | | | LCILLHAFRIRAVTIDRVMSYLNA | | | | | | | TTLESMTEDLNL |
| | | | | | | S | | | | | | | DSPLTPELNEILD |
| | | | | | | | | | | | | | TFLNDECLLHAM |
| | | | | | | | | | | | | | HISTGLSIFDTSLF |
| | SEQ ID | | | 219 | 290 | 293 | | | | 296 | 320 | 321 | 325 |
| | NO | | | | | | | | | | | | |
| | | | | | | | YB TATA | | | | | | |
| | | | | | | | 4X | | | | | | |
| SB | | | | | | | ZF10-1 | | | SV40 | | | |
| 04599 | | | | | | IL12p70 | BD | A2 | SFFV | NLS | ZF10-1 DBD | NS3 | mini VPR |
| | sIL12 | Lenti | DNA | | | ATGTGCCATCAGCAACTCGTCAT | cgggtttcgt | ACAA | gtaacgcca | ATG | GCAGACGGCACGG | CATCGGCAGCAGC | GATATGCTCGG |
| | YB_TATA | | | | | CTCCTGGTTCTCCCTTGTGTTCCT | aacaatcgc | TGGC | ttttgcaagg | CCC | TCCCGGCCTGGCG | TTGTGATCGTGGG | GACTTTGACCTG |
| | ZFBD | | | | | CGCTTCCCCTCTGGTCGCCATTT | atgaggattc | TGGC | catggaaaa | AAG | AGAGGCCTTTCCA | GAGGATGTCGTGT | GACGCTCTTGAT |
| | (syn | | | | | GGGAACTGAAGAAGGACGTCTA | gcaacgcct | CCAT | ataccaaac | AAG | GTGCAGAATCTGC | GCTGCCACAGCAT | ATCAGATGCCCT |
| | prmoter)- | | | | | CGTGGTCGAGCTGGATTGGTACC | tcGGCGT | AGTA | caagaatag | AAG | ATGCGGAACTTCA | CTACGGAAAGAAG | GGACGATTTCG |
| | A2 (insu- | | | | | CGGACGCCCCTGGAGAAATGGT | AGCCG | AATG | agaagttca | CGG | CCTGGACAGACAC | AAGGGCGACATCG | ATCTGGACATGT |
| | lator)- | | | | | CGTGCTGACTTGCGATACGCCAG | ATGTCG | CCGT | gatcaaggg | AAG | ACCAGAACACACA | ACACCTATCGGTA | TGGGGTCTGATG |
| | SV40 (pro- | | | | | AAGAGGACGGCATAACCTGGAC | CGctcccg | GTTA | cgggtacat | GTT | CAGGCGAGAAACC | GGCACAGGCTGTG | CTCTCGACGACT |
| | moter)- | | | | | CCTGGATCAGAGCTCCGAGGTG | tctcagtaaa | GTGT | gaaaatagc | | CTTCCAGTGCCGG | CAGAATCGTGCTG | TCGATCTGGATA |
| | Syn TF | | | | | CTCGGAAGCGGAAAGACCCTGA | ggtcGGC | GTTA | taacgttggg | | ATCTGTATGAGAA | AGCGGCTCTGGAA | TGCTTGGAAGTG |
| | (NLS + | | | | | CCATTCAAGTCAAGGAGTTCGG | GTAGCC | GTTG | ccaaacagg | | ATTTCAGCGACCA | CAAGCGCCCCTAT | ACGCGCTGGAT |
| | ZFBD | | | | | CGACGCGGGCCAGTACACTTGC | GATGTC | CTGT | atatctgcgg | | CAGCAGCCTGAAG | CACAGCCTACGCT | GATTTCGACCTT |
| | DNA | | | | | CACAAGGGTGGCGAAGTGCTGT | GCGcaatc | TCTTC | tgagcagttt | | CGGCACCTGAGAA | CAGCAGACAAGA | GACATGCTCATC |
| | binding | | | | | CCCACTCCCTGCTGCTGCTGCAC | ggactgcctt | CACG | cggccccg | | CCCATACCGGCAG | GGCCTGCTGGGCT | AATTCTCGATCC |
| | domain + | | | | | AAGAAAGAGGATGGAATCTGGT | cgtacGG | TCAG | gcccgggg | | CCAGAAACCATTT | GCATCATCACAAG | AGTGGAAGCCC |
| | NS3 | | | | | CCACTGACATCCTCAAGGACCA | CGTAGC | AAGA | ccaagaaca | | CAGTGTAGGATAT | CCTGACCGGCAGA | GAAAAAGAAAC |
| | protease + | | | | | AAAAGAACCGAAGAACAAGACC | CGATGT | GGCA | gatggtcac | | GCATGCGCAATTT | GACAAGAACCAG | GCAAGGTGGGA |
| | miniVPR | | | | | TTCCTCCGCTGCGAAGCCAAGA | CGCGcgt | CAGA | cgcagtttcg | | CTCCGTGCGGCAC | GTGGAAGGCGAG | AGTGGGGGCGG |
| | activation | | | | | ACTACAGCGGTCGGTTCACCTGT | atcagtcgc | CAAA | gccccggcc | | AACCTGACCAGAC | GTGCAGATCGTGT | CTCCGGTGGGA |
| | domain) | | | | | TGGTGGCTGACGACAATCTCCAC | ctcggaac | TTAC | cgaggccaa | | ACCTGAGGACACA | CTACAGCTACCCA | GCGGTAGTGTAT |
| | | | | | | CGACCTGACTTTCTCCGTGAAGT | GGCGTA | CACC | gaacagatg | | CACCGGGGAGAA | GACCTTCCTGGCC | TGCCTCAAGCTC |
| | | | | | | CGTCACGGGGATCAAGCGATCC | GCCGAT | AGGT | gtccccaga | | GCCTTTTCAATGT | ACCTGTATCAATG | CCGCGCCCGCTC |
| | | | | | | TCAGGGCGTGACCTGTGGAGCC | GTCGCG | GGCG | tatggccca | | CGCATATGCATGA | GCGTGTGCTGGGC | CTGCTCCGGCAA |
| | | | | | | GCCACTCTGTCCGCCGAGAGAG | cattogtaag | CTCA | accctcagc | | GAAACTTCTCTGA | CGTGTATCACGGC | TGGTTTCAGCTC |
| | | | | | | TCAGGGGAGACAACAAGGAATA | aggctcact | GAGT | agtttcttaag | | CCACTCCAACCTG | GCTGGCACAAGAA | TGGCACAAGCT |
| | | | | | | TGAGTACTCCGTGGAATGCCAG | ctcccttaca | CTGC | acccatcag | | AGCCGCCACCTCA | CAATCGCCTCTCC | CCAGCTCCAGTG |
| | | | | | | GAGGACAGCGCCTGCCCTGCCG | cggagtgga | GGAG | atgtttccag | | AAACCCACACCGG | AAAGGGCCCCGTG | CCTGTGCTCGCC |
| | | | | | | CGGAAGAGTCCCTGCCTATCGA | taACTAG | GCAT | gctccccca | | CTCTCAAAAGCCC | ATCCAGATGTACA | CCTGGCCCTCCG |
| | | | | | | GGTCATGGTCGATGCCGTGCATA | TTCTAG | CACA | aggacctga | | TTCCAATGTAGAA | CCAACGTGGACCA | CAGGCCGTAGC |
| | | | | | | AGCTGAAATACGAGAACTACAC | AGGGT | ACAG | aatgaccct | | TATGTATGAGGAA | GGACCTCGTTGGC | ACCTCCCGCCCC |
| | | | | | | TTCCTCCTTCTTTATCCGCGACA | ATATAA | CCCT | gcgccttattt | | CTTTAGCCAGCGG | TGGCCTGCTCCTC | CAAACCGACGC |
| | | | | | | TCATCAAGCCTGACCCCCCCAAG | TGGGG | GAAT | gaattaacca | | AGCAGCCTCGTGC | AAGGCAGCAGAA | AAGCCGGTGAG |
| | | | | | | AACTTGCAGCTGAAGCCACTCA | GCCA | TTGA | atcagcctg | | GCCATCTGAGAAC | GCCTGACACCTTG | GGGACTCTCTCT |
| | | | | | | AGAACTCCCGCCAAGTGGAAGT | | ATCC | cttctcgcttc | | TCACACTGGCGAA | CACCTGTGGCTCC | GAAGCCTTGCTG |
| | | | | | | GTCTTGGGAATATCCAGACACTT | | TGCT | tgttcgcgcg | | AAGCCGTTTCAAT | AGCGATCTGTACC | CAGCTTCAGTTC |
| | | | | | | GGAGCACCCCGCACTCATACTTC | | CTGC | cttctgcttcc | | GCCGTATCTGTAT | TGGTCACCAGACA | GATGATGAAGA |
| | | | | | | TCGCTCACTTTCTGTGTGCAAGT | | CACT | cgagctctat | | GCGCAACTTTAGC | CGCCGACGTGATC | TCTGGGCGCGCT |
| | | | | | | GCAGGGAAAGTCCAAACGGGAG | | GCCT | aaaagagct | | GAGAGCGGCCACC | CCTGTCAGAAGAA | CTTGGGGAACA |
| | | | | | | AAGAAAGACCGGGTGTTCACCG | | AGTT | cacaacccc | | TGAAGAGACATCT | GAGGGGATTCCAG | GCACGGATCCG |
| | | | | | | ACAAAACCTCCGCCACTGTGATT | | GAGA | tcacteggc | | GCGCACACACCTG | AGGCAGCCTGCTG | GCAGTATTTACG |
| | | | | | | TGTCGGAAGAACGCGTCAATCA | | CCTTT | gcgccagtc | | AGAGGCAGC | AGCCCTAGACCTA | GACCTCGCATCA |
| | | | | | | GCGTCCGGGCGCAGGATAGATA | | TACT | ctccgacag | | | TCAGCTACCTGAA | GTTGACAATAGT |
| | | | | | | CTACTCGTCCTCCTGGAGCGAAT | | ACCT | actgagtcg | | | GGGCAGCTCTGGC | GAATTTCAACA |
| | | | | | | GGGCCAGCGTGCCTTGTTCCGGT | | GACT | cccggg | | | GGACCTCTGCTTT | ACTTCTTAACCA |
| | | | | | | GGCGGATCAGGCGGAGGTTCAG | | AGCT | | | | GTCCTGCTGGACA | GGGAATACCGG |
| | | | | | | GAGGAGGCTCCGGAGGAGGTTC | | GAGA | | | | TGCCGTGGGCCTG | TTGCGCCCCATA |
| | | | | | | CCGGAACCTCCCTGTGGCAACCC | | CATT | | | | TTTAGAGCCGCCG | CGACGGAACCT |
| | | | | | | CCGACCCTGGAATGTTCCCGTGC | | TACG | | | | TGTGTACAAGAGG | ATGCTGATGGA |
| | | | | | | CTACACCACTCCCAAAACCTCCT | | ACAT | | | | CGTGGCCAAAGCC | GTACCCTGAAG |
| | | | | | | GAGGGCTGTGTCGAACATGTTG | | TTAC | | | | GTGGACTTCATCC | CTATAACCAGA |
| | | | | | | CAGAAGGCCCGCCAGACCCTTG | | TGGC | | | | CCGTGGAAAACCT | CTCGTAACTGGC |
| | | | | | | AGTTCTACCCCTGCACCTCGGAA | | TCTA | | | | GGAAACCACCATG | GCCCAACGCCC |
| | | | | | | GAAATTGATCACGAGGACATCA | | GGAC | | | | CGGAGCCCCGTGT | GCCCGACCCGG |
| | | | | | | CCAAGGACAAGACCTCGACCGT | | TCAT | | | | TCACCGACAATTC | CTCCTGCGCCGC |
| | | | | | | GGAAGCCTGCCTGCCGCTGGAA | | TTTAT | | | | TAGCCCTCCAGCC | TGGGTGCGCCG |
| | | | | | | TGAACTCCCGCGAGACAAGCTTT | | TTCA | | | | ACCCCATCACCAA | GGTCTTCTCTCA |
| | | | | | | CTGACCAAGAACGAATCGTGTC | | TCAT | | | | GTGACACTGACAC | GGTCTTCCGAAT |
| | | | | | | TGAACTCCCGCGAGACAAGCTTT | | TTCA | | | | ACCCCATCACCAA | GGTCTTCTCTCA |
| | | | | | | ATCACTAACGGCAGCTGCCTGG | | TTAC | | | | GATCGACAGAGAG | GGGGACGAAGA |
| | | | | | | CGTCGAGAAAGACCTCATTCAT | | TTTTT | | | | GTGCTGTACCAAG | TTTCAGTTCCAT |
| | | | | | | GATGGCGCTCTGTCTTTCCTCGA | | TTTTC | | | | AGTTCGACGAGAT | TGCGGATATGG |
| | | | | | | TCTACGAAGATCTGAAGATGTAT | | TTTG | | | | GGAAGAGTGCAGC | ACTTTTCCGCGC |
| | | | | | | CAGGTCGAGTTCAAGACCATGA | | AGAC | | | | CAGCAC | TCCTGAGTGGG |
| | | | | | | ACGCCAAGCTGCTCATGGACCC | | GGAA | | | | | GGTGGCTCTGG |
| | | | | | | GAAGCGGCAGATCTTCCTGGAC | | TCTC | | | | | AGGCTCTGGTTC |
| | | | | | | CAGAATATGCTCGCCGTGATTGA | | GCTC | | | | | CGACCTCAGCC |
| | | | | | | TGAACTGATGCAGGCCCTGAATT | | T | | | | | ATCCTCCACCGA |
| | | | | | | TCAACTCCGAGACTGTGCCTCAA | | | | | | | GAGGACACCTC |
| | | | | | | AAGTCCAGCCTGGAAGAACCGG | | | | | | | GACGAGCTGAC |
| | | | | | | ACTTCTACAAGACCAAGATCAA | | | | | | | AACCACCCTCG |
| | | | | | | GCTGTGCATCCTGTTGCACGCTT | | | | | | | AAAGTATGACG |
| | | | | | | TCCGCATTCGAGCCGTGACCATT | | | | | | | GAAGATCTGAA |
| | | | | | | GACCGCGTGATGTCCTACCTGAA | | | | | | | CTTGGATTCCCC |
| | | | | | | CGCCAGT | | | | | | | CCTTACCCCAGA |
| | | | | | | | | | | | | | ACTGAATGAAA |
| | | | | | | | | | | | | | TCCTCGATACGT |
| | | | | | | | | | | | | | TCTTGAACGATG |
| | | | | | | | | | | | | | AGTGCCTTTTGC |
| | | | | | | | | | | | | | ACGCCATGCAT |
| | | | | | | | | | | | | | ATATCAACAGG |
| | | | | | | | | | | | | | TTTGTCTATCTT |
| | | | | | | | | | | | | | CGACACGTCCCT |
| | | | | | | | | | | | | | CTTTTGA |
| | SEQ ID | | | | | 57 | 299 | 300 | 17 | 297 | 341 | 342 | 343 |
| | NO | | | | | | | | | | | | |
| | | | AA | | | MCHQQLVISWFSLVFLASPLVAIW | | | | MPK | SRPGERPFQCRICM | EDVVCCHSIYGKK | DALDDFDLDML |
| | | | | | | ELKKDVYVVELDWYPDAPGEMV | | | | KKR | RNFSRRHGLDRHT | KGDIDTYRYIGSSG | GSDALDDFDLD |
| | | | | | | VLTCDTPEEDGITWTLDQSSEVLG | | | | KV | RTHTGEKPFQCRIC | TGCVVIVGRIVLSG | MLGSDALDDFDL |
| | | | | | | SGKTLTIQVKEFGDAGQYTCHKG | | | | | MRNFSDHSSLKRH | SGTSAPITAYAQQT | DMLGSDALDDF |
| | | | | | | GEVLSHSLLLLHKKEDGIWSTDIL | | | | | LRTHTGSQKPFQCR | RGLLGCIITSLTGRD | DLDMLINSRSSG |
| | | | | | | KDQKEPKNKTFLRCEAKNYSGRF | | | | | ICMRNFSVRHNLTR | KNQVEGEVQIVST | SPKKKRKVGSGG |
| | | | | | | TCWWLTTISTDLTFSVKSSRGSSD | | | | | HLRTHTGEKPFQCR | ATQTFLATCINGVC | GSGGSGSVLPQA |
| | | | | | | PQGVTCGAATLSAERVRGDNKEY | | | | | ICMRNFSDHSNLSR | WAVYHGAGTRTIA | PAPAPAPAMVSA |
| | | | | | | EYSVECQEDSACPAAEESLPIEVM | | | | | HLKTHTGSQKPFQ | SPKGPVIQMYTNV | LAQAPAPVPVLA |
| | | | | | | VDAVHKLKYENYTSSFFIRDIIKPD | | | | | CRICMRNFSQRSSL | DQDLVGWPAPQGS | PGPPQAVAPPAP |
| | | | | | | PPKNLQLKPLKNSRQVEVSWEYP | | | | | VRHLRTHTGEKPF | RSLTPCTCGSSDLY | KPTQAGEGTLSE |
| | | | | | | DTWSTPHSYFSLTFCVQVQGKSK | | | | | QCRICMRNFSESGH | LVTRHADVIPVRRR | ALLQLQFDDEDL |
| | | | | | | REKKDRVFTDKTSATVICRKNASI | | | | | LKRHLRTHLRGS | GDSRGSLLSPRPISY | GALLGNSTDPAV |
| | | | | | | SVRAQDRYYSSSWSEWASVPCSG | | | | | | LKGSSGGPLLCPAG | FTDLASVDNSEF |
| | | | | | | GGSGGGSGGGSGGGSRNLPVATP | | | | | | HAVGLFRAAVCTR | QQLLNQGIPVAP |
| | | | | | | DPGMFPCLHHSQNLLRAVSNMLQ | | | | | | GVAKAVDFIPVENL | HTTEPMLMEYPE |
| | | | | | | KARQTLEFYPCTSEEIDHEDITKD | | | | | | ETTMRSPVFTDNSS | AITRLVTGAQRP |
| | | | | | | KTSTVEACLPLELTKNESCLNSRE | | | | | | PPAVTLTHPITKIDR | PDPAPAPLGAPG |
| | | | | | | TSFITNGSCLASRKTSFMMALCLS | | | | | | EVLYQEFDEMEEC | LPNGLLSGDEDF |
| | | | | | | SIYEDLKMYQVEFKTMNAKLLM | | | | | | SQH | SSIADMDFSALLS |
| | | | | | | DPKRQIFLDQNMLAVIDELMQAL | | | | | | | GGGSGGSGSDLS |
| | | | | | | NFNSETVPQKSSLEEPDFYKTKIK | | | | | | | HPPPRGHLDELT |
| | | | | | | LCILLHAFRIRAVTIDRVMSYLNA | | | | | | | TTLESMTEDLNL |
| | | | | | | S | | | | | | | DSPLTPELNEILD |
| | | | | | | | | | | | | | TFLNDECLLHAM |
| | | | | | | | | | | | | | HISTGLSIFDTSLF |
| | SEQ ID | | | | | 293 | | | | 296 | 354 | 321 | 325 |
| | NO | | | | | | | | | | | | |

### Example 11: Assessment of crIL15 Effects in CAR-NK Cells

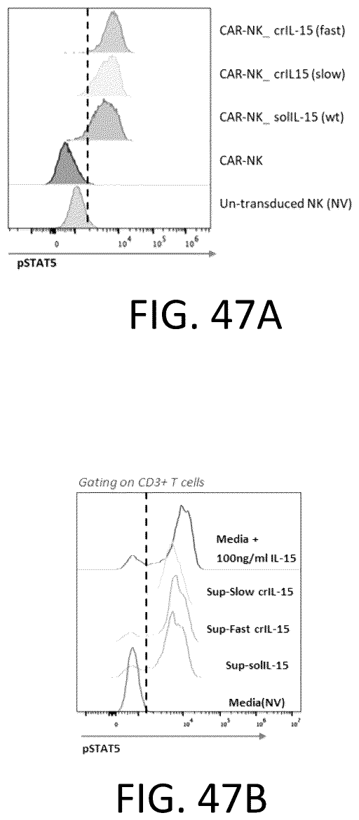

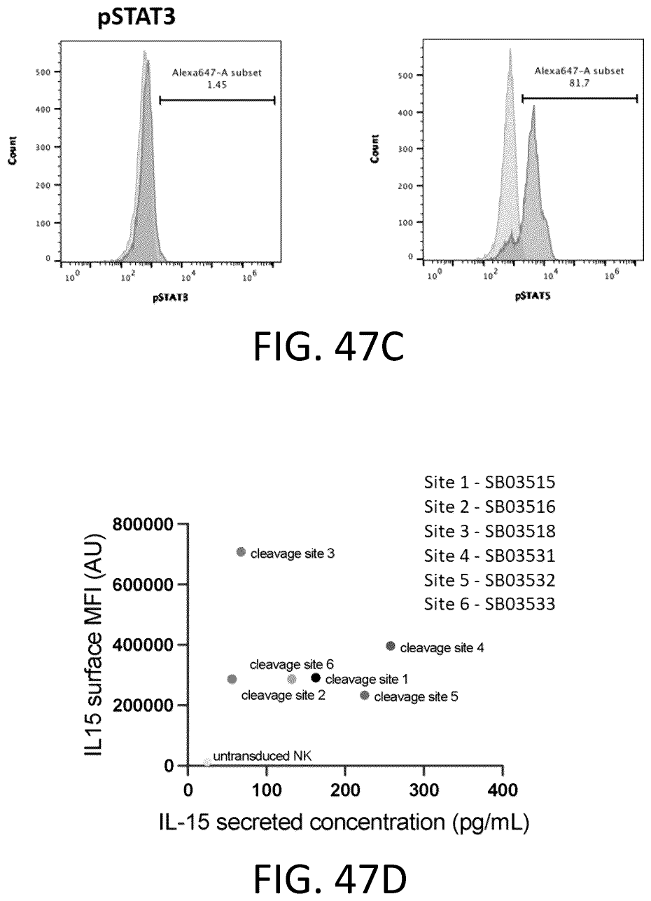

NK cells were transduced with virus expressing a bi-cistronic construct consistent of an activating CAR and various forms of IL15 including wt (wild type or naïve, fully secreted IL15 (SEQ ID NO: 357)) or two different isoforms of calibrated-release IL15 with different kinetics (“slow” crIL15 (SEQ ID NO: 355) or “fast” (optimized TACE cleavage site) crIL15 (SEQ ID NO: 356). 3 days after transduction, NK cells were washed to remove any cytokines from the media, and cytokine-starved for a period of 24 to 48 hours. NK cells were collected and the levels of phosphorylated STAT5 (pSTAT5) were determined via flow cytometry. In parallel, the conditioned media from different transduced NK cells was collected and used to incubate resting PBMCs. The levels of pSTAT5 in CD3+ T cells from the resting PBMCs were quantified. In a similar experiment, NK cells were transduced with virus expressing various forms of crIL-15 with different cleavage sites (e.g., SB03515, SB03516, SB03518, SB03531, SB03532, SB03533). After culture for a number of days, supernatant was collected from the cultures and secreted IL-15 was measured by ELISA. Transduced NK cells were stained with fluorescently labeled antibodies against IL-15 and surface IL-15 expression was measured by flow cytometry (MFI: geometric mean of the IL-15 stain fluorescence signal across the population). Furthermore, pSTAT3 and pSTAT5 levels were determined via flow cytometry. Results are shown in FIGS. 47A-D. Various embodiments of crIL-15, and their corresponding SB construct numbers, are described in PCT Publication WO2022098922A1, which is hereby incorporated by reference in its entirety. Results showed that crIL15 constructs provide autocrine (FIG. 47A, 47C) and paracrine (FIG. 47B) effects acting on the engineered CAR-NK cells and in co-cultured PBMCs, and that different engineered forms of crIL15 exhibit different relative levels of surface-associated and secreted IL15 (FIG. 47D).

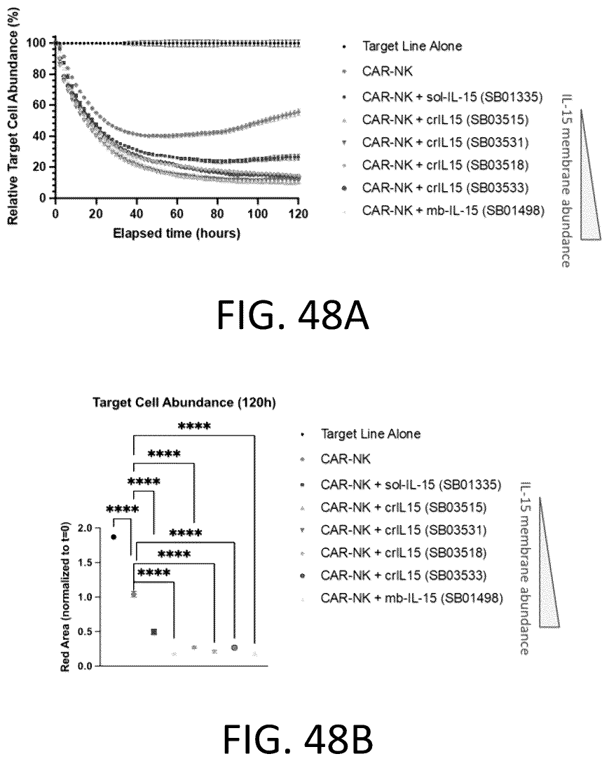

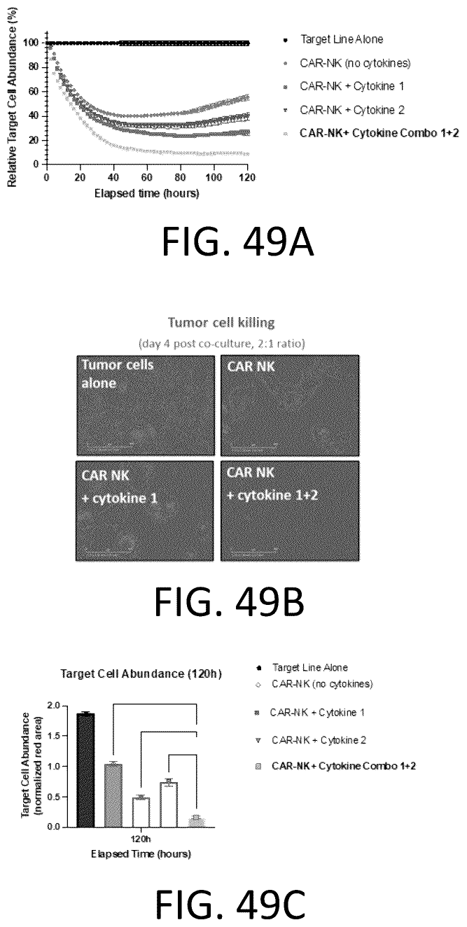

To assess target cell killing of CAR-NK cells expressing crIL15, NK cells were transduced with virus expressing an activating CAR and different forms of IL15 including membrane-bound (mbIL15; SEQ ID NO:358), secreted (wild type, naïve; SEQ ID NO: 357) or different forms of crIL15 (“slow” crIL15 (SEQ ID NO: 355) or “fast” (optimized TACE cleavage site) crIL15 (SEQ ID NO: 356)). 10 days after transduction, NK cells were collected, counted and seeded in a co-culture with tumor target cells that constitutively express a fluorescent reporter (mKate) at the appropriate effector to target (E:T) ratios. Tumor target cell growth (area) was quantified using an imaging-based system (Incucyte, Sartorius). Target cell area was quantified and compared across different conditions. Target cell area is normalized by the target alone growth and represented as percentages. Results showed that expression of crIL15 increased target cell killing by CAR-NK cells (FIGS. 48A and 48B). Furthermore, co-expression of multiple cytokines in CAR-NK cells lead to a significant increase in target cell killing compared to co-expression of a single cytokine (FIGS. 49A-49C).

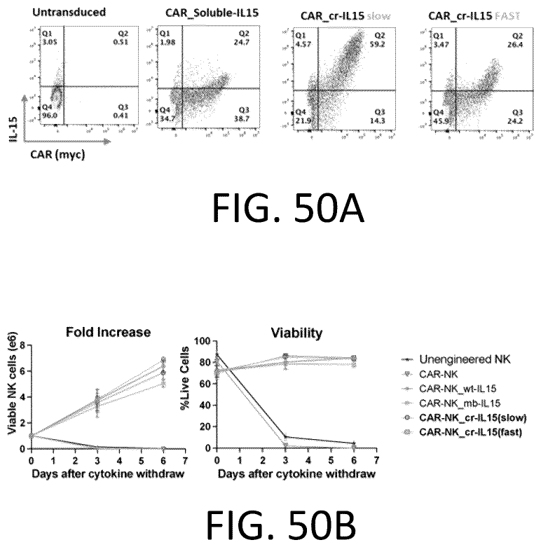

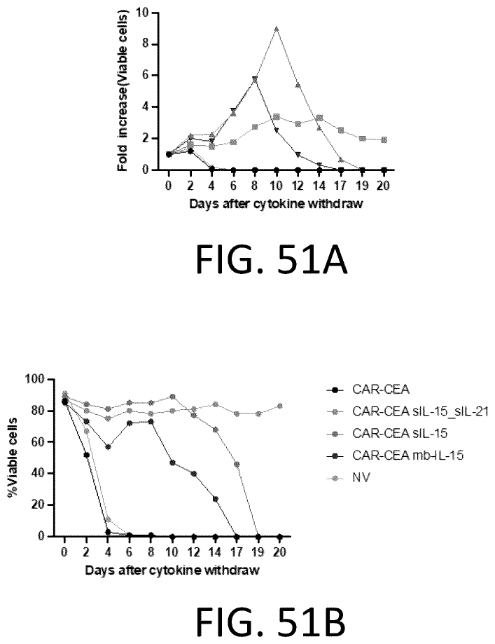

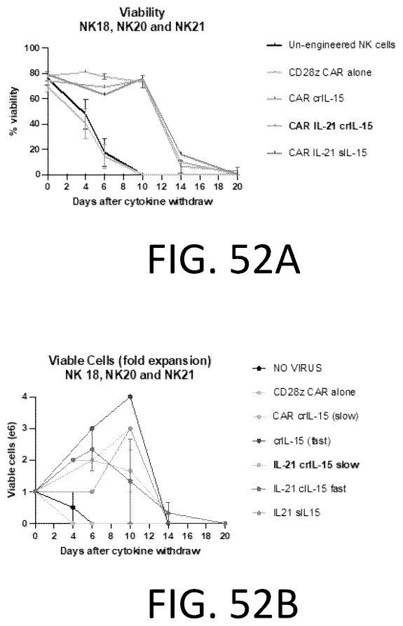

To further optimize the kinetic properties of crIL15, NK cells were transduced with virus expressing a bi-cistronic construct consistent of an activating CAR and various forms of IL15 including wild type or naïve, fully secreted IL15 (SEQ ID NO: 357) or two different isoforms of calibrated-release IL15 with different kinetics (“slow” crIL15 (SEQ ID NO: 355) or “fast” IL15 (SEQ ID NO: 356)). In some experiments, IL15 was co-expressed with secreted IL21 (SEQ ID NO: 359). 3 days after transduction, NK cells were washed to remove any cytokines from the media, and seeded in the absence of any recombinant cytokines. NK cells were counted every 2-3 days to determine their growth and survival. NK cells were collected and the levels of phosphorylated STAT5 (pSTAT5) were determined via flow cytometry. “Slow” crIL15 showed a higher proportion of IL15 on the cell membrane compered to “fast” crIL15 and soluble IL15 (sIL15) (FIG. 50A). However, both “slow” and “fast” forms of IL15 showed similar effects on NK cell expansion and viability (FIG. 50B). Additionally, in longer time-course experiments, the results showed that a combination of co-expression of IL21 with IL15 yielded sustained expansion (FIG. 51A) and viability (FIG. 51B) of CAR-NK cells, whereas cells co-expressing IL15 alone showed a peak in expansion after 8-12 days followed by a decrease in viability over time. Co-expression of IL21 with crIL15 yielded sustained viability in the absence of soluble cytokines in culture medium (FIGS. 52A-52B).

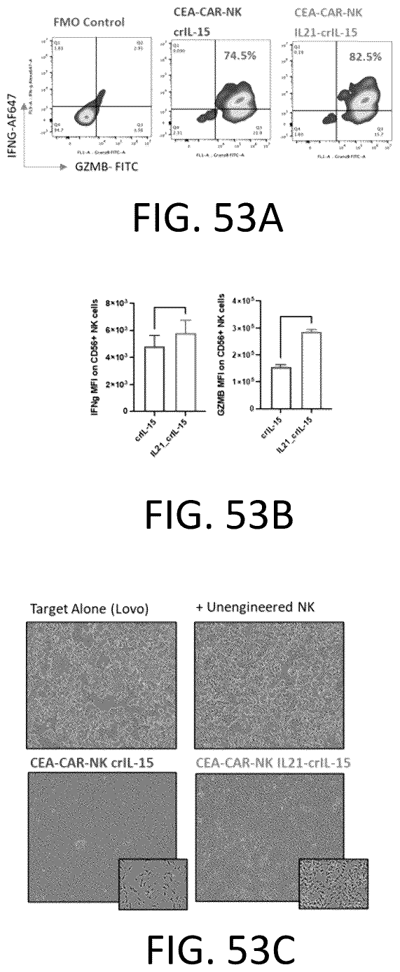

To assess, target cell killing by CAR-NK cells co-expressing crIL15 and IL21, NK cells were transduced with virus expressing a bi-cistronic or tri-cistronic constructs consistent of an activating CAR and crIL15 in combination with IL21. 3 days after transduction, NK cells were washed to remove any cytokines from the media and seeded in the absence of any recombinant cytokines. 3 days later, NK cells were co-cultured with tumor target cells (Ls174t or Lovo). NK:target cell co-cultures were assessed using flow cytometry and NK cell activation cytokines (IFNg and GZMB) were quantified. Right panel: tumor target cell area was quantified using an image-based assay (Incucyte). Representative images after two rounds of serial killing. Co-expression of crIL15 and IL21 lead to increased activation of CAR-NK cells (FIGS. 53A and 53B) and improved expansion and persistence of the NK cells in serial killing assays FIG. 53C) compared to crIL15 alone.

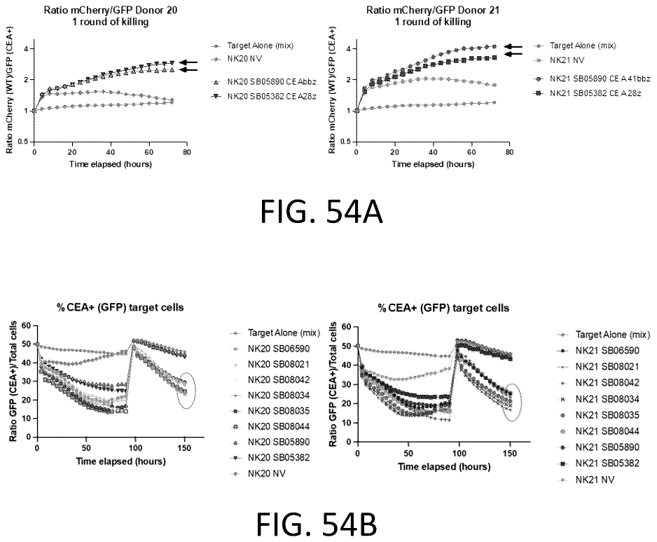

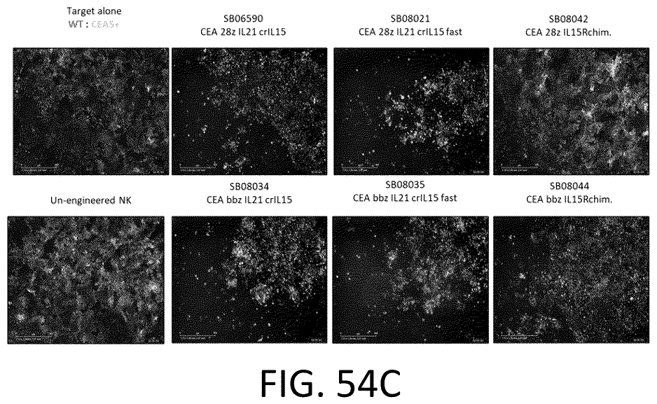

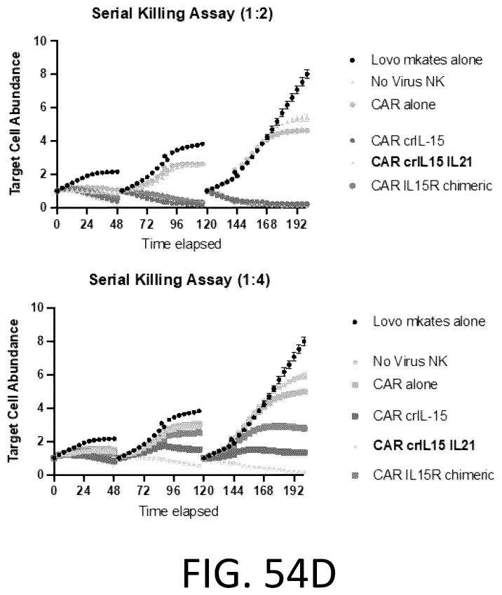

To ensure that CAR-NK cells maintain target specificity, NK cells were transduced with retrovirus encoding different bi or tri-cistronic constructs consistent on a CEA-activating CAR with different intracellular signaling domains (hMN14-28z (SEQ ID NO 362) or hMN14-BBz (SEQ ID NO: 364)) and IL15 (“slow” crIL15 (SEQ ID NO: 355), “fast” IL15 (SEQ ID NO: 356), or sushi crIL15 (SEQ ID NO:361) and IL21 (SEQ ID NO:359). 6 to 7 days after transduction, NK cells were collected, counted and seeded in a co-culture with tumor target cells that constitutively express a nuclear fluorescent reporter at the appropriate effector to target (E:T) ratios. Targets were a 1:1 mixture of non-antigen expressing (in red, mCherry) or antigen-expressing (in green, GFP) to discriminate between antigen-specific and non-antigen-specific killing. Tumor target cell growth was quantified using an imaging-based system (Incucyte, Sartorius). After 72 hours of co-culture, new target cells were plated, and the NK cells were collected from the original plates and transferred to newly seeded targets for a 2^{nd} tumor re-challenge (serial killing). Ratio between mCherry+ and GFP+ target cells was quantified to determine antigen-specificity. Results showed that CAR-NK cells (either using a CD28z or 41BBz signaling domains) specifically killed target-expressing cells (FIG. 54A) and that this specificity was maintained when cytokines (various forms of crIL15 with “slow” or “fast” cleavage or the combination of crIL15 and IL21) were co-expressed with the CAR over two serial rounds of target cell killing (FIG. 54B). Representative images of control and antigen-expressing cells at the end of the serial killing assay are shown in FIG. 54C. Serial killing results from CAR-NK cells using 41BBz signaling domains, at various E:T ratios, are further shown in FIG. 54D.

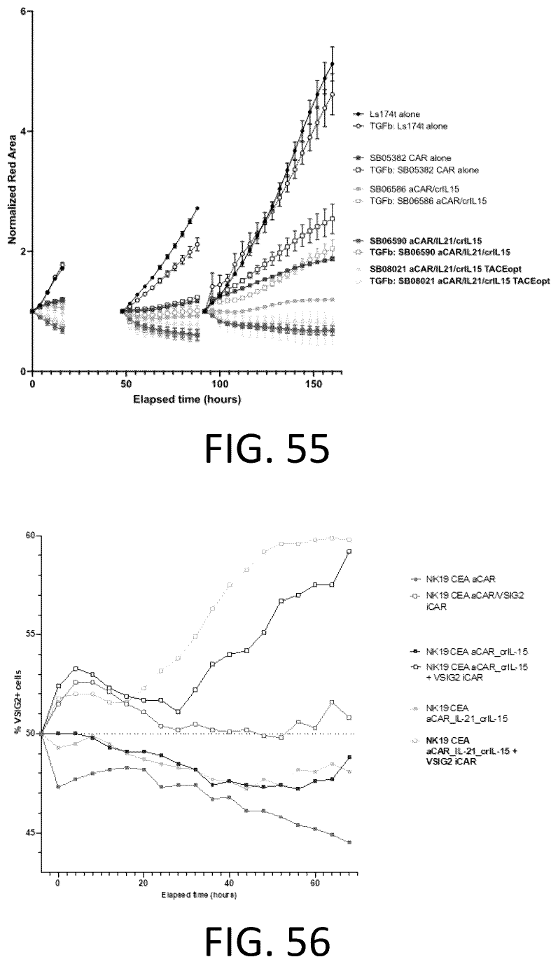

To assess whether CAR-NK cells co-expressing crIL15 and IL21 can maintain target cell killing activity in immunosuppressive conditions, NK cells were transduced with retrovirus encoding different bi or tri-cistronic constructs consistent on a CEA-activating CAR with CD28z intracellular domain (hMN14-28z (SEQ ID NO 362)) and IL15 (“slow” crIL15 (SEQ ID NO: 355) or “fast” IL15 (SEQ ID NO: 356)) and IL21 (SEQ ID NO: 359). 6 to 7 days after transduction, NK cells were harvested, counted and seeded in a co-culture with CEA+ tumor target cells that constitutively express a fluorescent reporter (mKate) at the appropriate effector to target (E:T) ratios. TGFb was added to the co-culture at 20 pg/mL in the appropriate conditions. Tumor target cell growth (area) was quantified using an imaging-based system (Incucyte, Sartorius). After 48 to 72 hours of co-culture, new target cells were plated, and the NK cells were collected from the original plates and transferred to newly seeded targets for a 2^{nd} or 3^{rd} tumor re-challenge (serial killing). TGFb was added to the co-culture at 20 pg/mL after each round. Target cell area was quantified and compared across different conditions. Results showed that serial killing capacity was maintained for CAR-NK cells co-expressing IL21 and crIL15 (FIG. 55).

An inhibitory CAR (iCAR; SEQ ID NO:366) that recognizes a protective antigen (VSIG2) was introduced to protect against on-target/off-tumor cell killing. NK cells were transduced with retrovirus encoding different bi or tri-cistronic constructs consistent on a CEA-activating CAR (hMN14-28z (SEQ ID NO 362)) and crIL15 (“slow” crIL15 (SEQ ID NO: 355)) and IL21 (SEQ ID NO:359). 6 to 7 days after transduction, NK cells were harvested, counted and seeded in a co-culture with tumor target cells that constitutively express a nuclear fluorescent reporter at the appropriate effector to target (E:T) ratios. Targets were a 1:1 mixture of Protective Antigen (VSIG2) expressing (in red, mCherry) or non-targeting Protective Antigen-expressing (HER2) (in green, GFP) to discriminate between Protective-antigen expressing and iCAR-mediated protection. Tumor target cell growth was quantified using an imaging-based system (Incucyte, Sartorius). Ratio between mCherry+ and GFP+ target cells was quantified to determine antigen-specificity and iCAR protective effects. Increased ratio of VSIG2+ cells indicates protection of such targets in an antigen-dependent manner when the iCAR is present. The data showed that introduction of the iCAR protected cells expressing the protective antigen from CAR-NK mediated cytotoxicity even when the CAR was co-expressed with cytokines crIL15 and IL21 (FIG. 56).

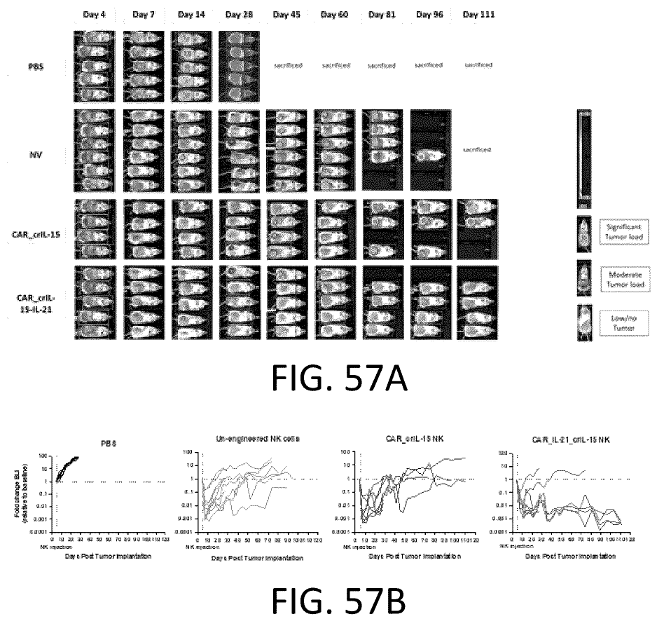

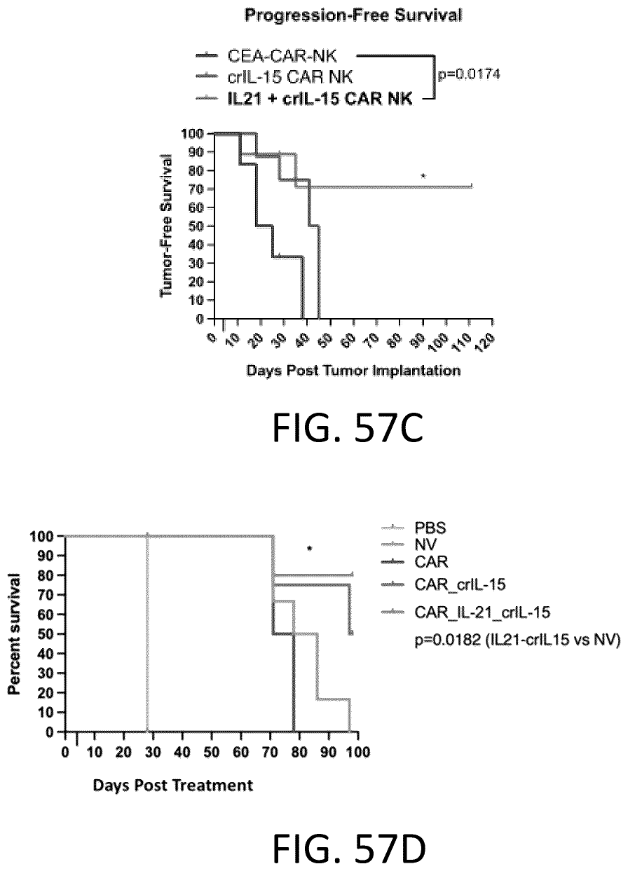

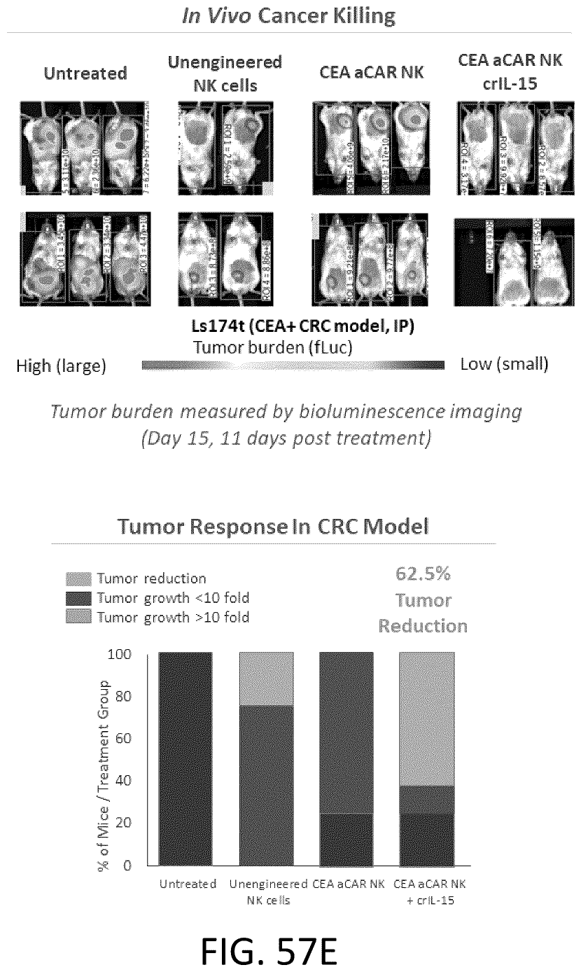

To measure the in vivo effects of CAR-NK cells co-expressing crIL15 and IL21, NK cells were transduced with retrovirus encoding different bi or tri-cistronic constructs consistent on a CEA-activating CAR (hMN14-28z (SEQ ID NO 362)) and “slow” crIL15 (SEQ ID NO: 355) with or without IL21 (SEQ ID NO:359). NSG female mice were implanted with LOVO (CEA+) human colorectal cancer cells (IP) that constitutively express the bioluminescence reporter fLuciferase. Mice were randomized prior to treatment based on tumor burden (measured by bioluminescence, BLI). NK cells were expanded and used to treat NSG mice with established LOVO human xenograft tumors on day 4 after tumor implant. Tumor burden was quantified at various time points using BLI (fLuciferase) of the tumor cells. fLuciferase intensity (representative of tumor burden) shown for all the mice that were on study and measured for survival. Progression-free survival was calculated as time until tumor burden (BLI) was the same as prior to treatment. Individual tumor burden progression curves are shown for each of the treatment groups. Values for each mice were normalized to starting BLI (tumor burden) values to calculate the fold change in tumor burden. Peritoneal fluid and whole blood was collected at the mentioned time points and NK cell presence was determined using flow cytometry. Representative plots showing mouse vs human (NK) cells are shown gating on live cells. Results show that treatment of mice with CAR-NK cells co-expressing crIL15 and IL21 yielded the greatest and most durable reduction in tumor burden (FIGS. 57A and 57B), increase in progression-free survival (FIG. 57C), and increase in percent survival (FIG. 57D) compared to control or CAR-NK cells co-expressing crIL15 alone. Progression free survival was assessed using BLI tumor burden values. When the fold change values became greater than or equal to 0.5 (New BLI value/First BLI value≥0.5) that animal was no longer deemed “progression-free” and the graph for their respective group decreases on that day toward y=0. Another experiment was performed to assess in vivo tumor suppression by CAR-NK cells with or without crIL15. NK cells were transduced with SB04285 (CEA aCAR) or SB05629 (crIL-15 CEA-aCAR-NK). On Day 0, 30e6 Ls174t-luc-mKate cells were administered intraperitoneally into 6-8 week old female NSG mice. On Day 4, mice received no NK cells, untransduced NK cells, CEA aCAR NK cells, or crIL-15 CEA-aCAR NK cells. Bioluminescence imaging to monitor tumor growth, body weight and clinical observations were performed to monitor animal health. Results are shown in FIG. 57E. As shown, CEA-CAR-NK cells armed with cr-IL15 provide an advantage in tumor control compared to CEA-CAR-NK alone up to 14 days post treatment.

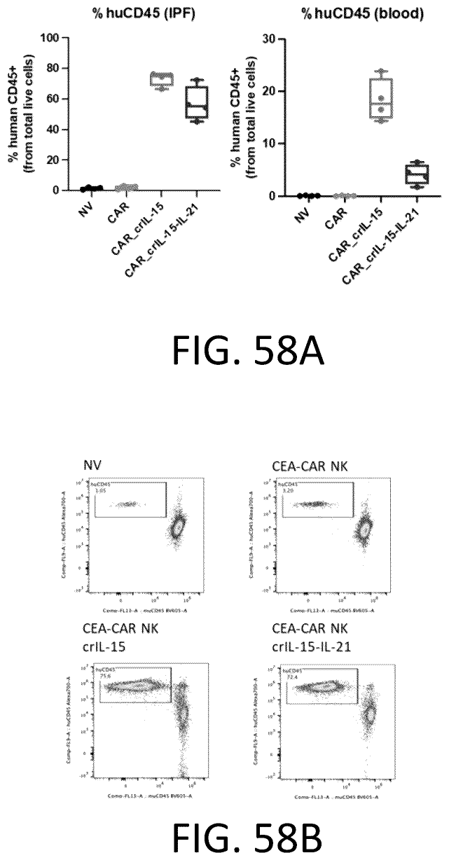

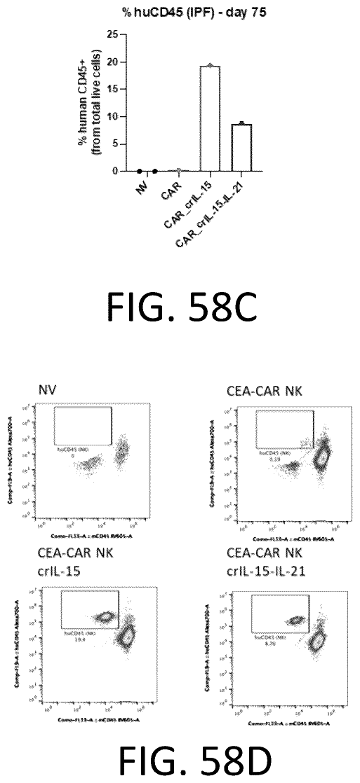

CAR-NK cells expressing crIL15 or crIL15+IL21 also showed increased persistence compared to control cells or cells expressing the CAR alone at 27 days (FIGS. 58A and 58B) and 70 days (FIGS. 58C and 58D) post-administration.

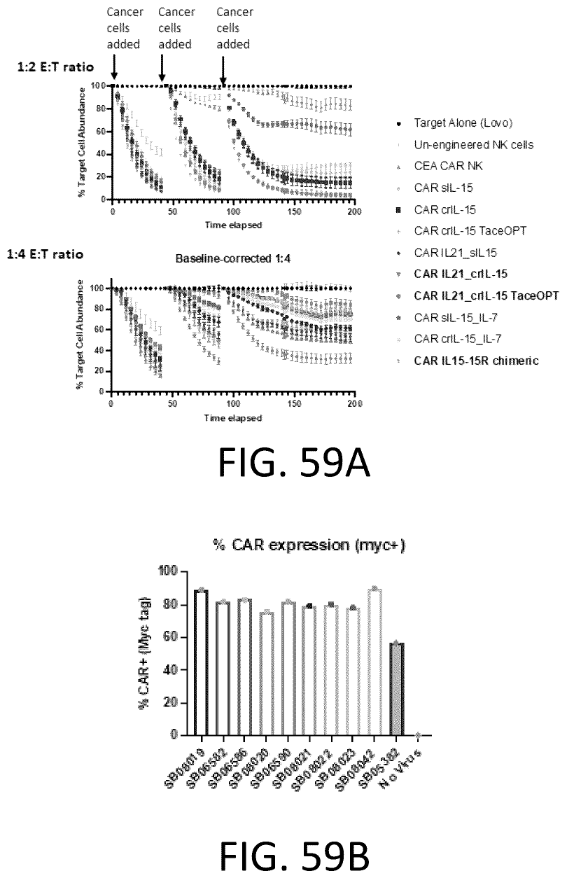

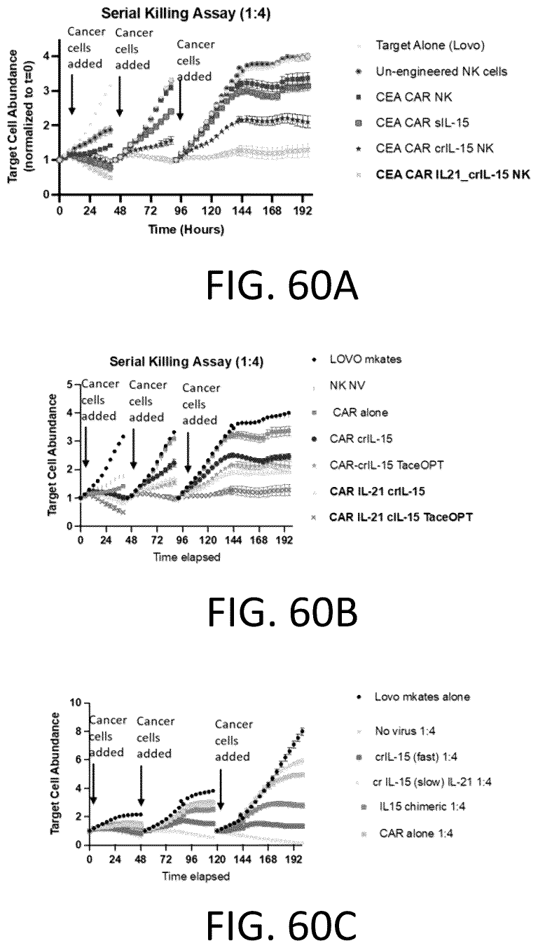

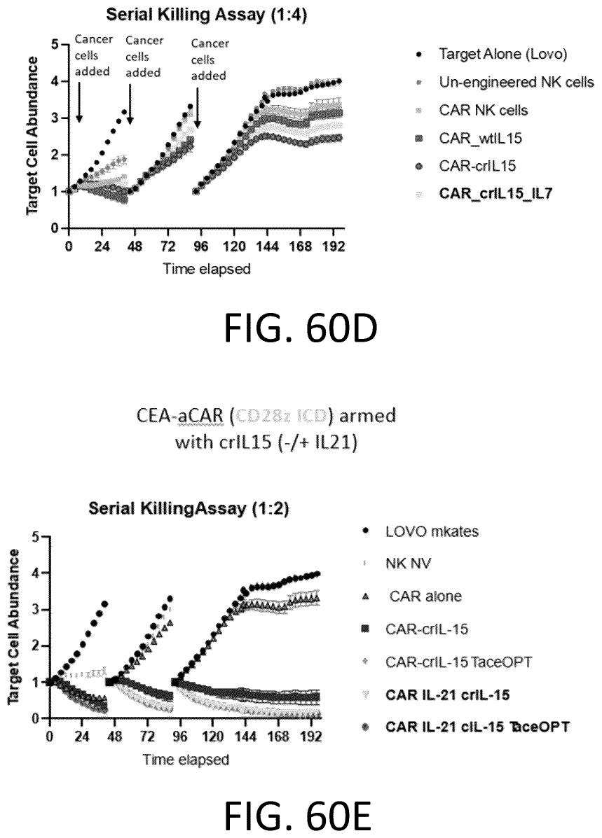

To test the construction of different formats of engineered IL15 constructs, NK cells were transduced with retrovirus encoding different bi or tri-cistronic constructs consistent on a CEA-activating CAR with different intracellular signaling domains (hMN14-28z (SEQ ID NO 362) or hMN14-BBz (SEQ ID NO: 364)) and cytokines IL15 (“slow” (SEQ ID NO: 355) or “fast”/TaceOPT IL15 (SEQ ID NO: 356)), IL21(SEQ ID NO:359), or IL7 (SEQ ID NO: 394) or a chimeric cytokine receptor (IL15-IL15Ra chimeric proteins; SEQ ID NOs: 361 and 391). 6 to 7 days after transduction, NK cells were collected, counted and seeded in a co-culture with tumor target cells that constitutively express a fluorescent reporter (mKate) at the appropriate effector to target (E:T) ratios. Tumor target cell growth (area) was quantified using an imaging-based system (Incucyte, Sartorius). After 48 to 72 hours of co-culture, new target cells were plated, and the NK cells were collected from the original plates and transferred to newly seeded targets for a 2^{nd} or 3^{rd} tumor re-challenge (serial killing). Target cell area was quantified and compared across different conditions. All tested constructs showed good transduction efficiency and comparable CAR expression (FIG. 59B) The combination of crIL15 with IL21 and a chimeric IL15-IL15R molecule with CD28 intracellular domain yielded the greatest serial target cell killing at both tested E:T ratios (FIG. 59A). When compared to wild-type (secreted) IL15, crIL15 expression in CAR-NK cells yielded superior serial target cell killing, which was enhanced by co-expression of IL21 (FIG. 60A). Additionally, the “slow” crIL15 yielded enhanced target cell killing compared to the “fast” (TaceOPT) crIL15, which was also enhanced by co-expression of IL21 (FIGS. 60B-60C). Similar serial target cell killing is shown at a different E:T ratio in FIG. 60E. Co-expression of IL7 with crIL15 showed no improvement in serial target cell killing when combined with crIL15 (FIG. 60D).

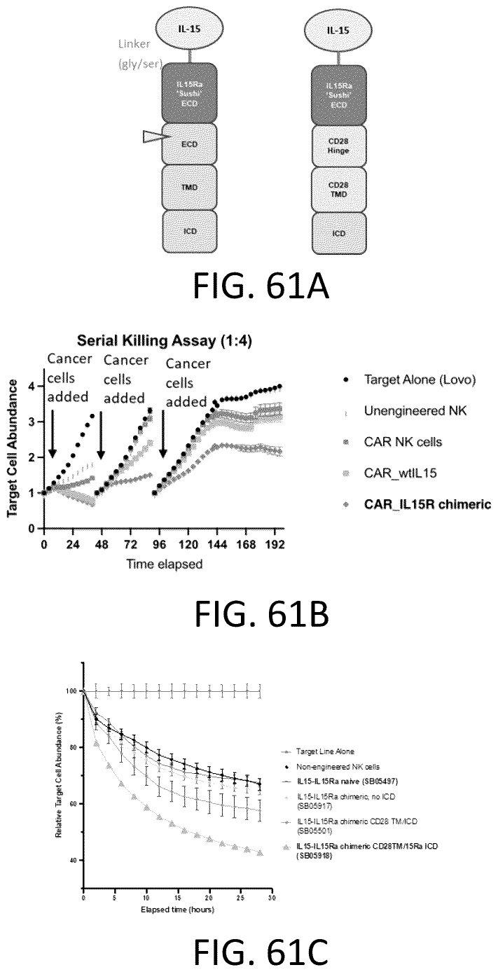

Additionally, various constructions of a chimeric protein containing IL15 tethered to the IL15 receptor (FIG. 61A) was tested. The chimeric protein comprising CD28 hinge and transmembrane domains yielded superior serial target cell killing, especially at the second and third rounds of killing (FIGS. 61B and 61C).

A summary of constructs used in this example are provided in Table 23.

TABLE 23

| Construct | |
|---|---|
| Number | All construct descriptions |
| SB04285 | aCAR: hMN 28 CD3z |
| SB05623 | aCAR: hMN 28 CD3z; P2A; IL15 |
| SB05624 | aCAR: hMN 28z; P2A; HA-mbIL15 |
| SB05625 | aCAR: hMN 28z; P2A; IL21 |
| SB05626 | aCAR: hMN 28z; P2A; IL15; E2A T2A IL21 |
| SB05627 | aCAR: hMN 28z; P2A; IL21; E2A T2A; IL15 |
| SB05628 | aCAR: hMN 28z; P2A; HA-crIL15 |
| SB05629 | aCAR: hMN 28z; P2A; HA-crIL15 |
| SB05657 | aCAR: hMN 28z; P2A; sushi crIL15 |
| SB05658 | aCAR: hMN OX40z; P2A; sushi crIL15 |
| SB05659 | aCAR: hMN OX40z; P2A; IL15 |
| SB05660 | aCAR: hMN OX40z; P2A; HA-mbIL15 |
| SB05661 | aCAR: hMN OX40z; P2A; IL21 |
| SB05662 | aCAR: hMN OX40z; P2A; IL15; E2A T2A; IL21 |
| SB05663 | aCAR: hMN OX40z; P2A; IL21; E2A T2A; IL15 |
| SB05664 | aCAR: hMN OX40z; P2A; HA-crIL15 |
| SB05665 | aCAR: hMN OX40z; P2A; HA-crIL15 |
| SB06582 | aCAR: hMN 28z; P2A; IL21; Furin E2AT2A; IL15 |
| SB06584 | aCAR: hMN 28z; E2AT2A; IL21; Furin E2AT2A; IL15 |
| SB06586 | aCAR: hMN 28z; P2A; HA-crIL15 |
| SB06588 | aCAR: hMN 28z; E2AT2A; HA-crIL15 |
| SB06590 | aCAR: hMN 28z; P2A; IL21 Furin E2AT2A; HA-crIL15 |
| SB06592 | aCAR: hMN 28z; E2AT2A; IL21; Furin E2AT2A; HA-crIL15 |
| SB08019 | aCAR: hMN 28z; P2A; sIL15 |
| SB08020 | aCAR: hMN 28z; P2A; HA- crIL15 (fast) |
| SB08021 | aCAR: hMN 28z; P2A; IL21 Furin E2AT2A; cIL15 |
| | “fast” (SB04802) |
| SB08022 | aCAR: hMN 28z; P2A; sIL15; Furin E2AT2A; IL7 |
| SB08023 | aCAR: hMN 28z; P2A; crIL15 (slow); E2AT2A; IL7 |
| SB08024 | aCAR: hMN OX40z; P2A; sIL15 |
| SB08025 | aCAR: hMN OX40z; P2A; IL21; Furin E2AT2A; IL15 |
| SB08026 | aCAR: hMN OX40z; P2A; HA- crIL15 (slow) |
| SB08027 | aCAR: hMN OX40z; P2A; HA- crIL15 (fast) |
| SB08028 | aCAR: hMN OX40z; P2A; crIL15 (slow); E2AT2A; IL21 |
| SB08029 | aCAR: hMN OX40z; P2A; crIL15 (fast); E2AT2A; IL21 |
| SB08030 | aCAR: hMN 41BBz; P2A; sIL15 |
| SB08031 | aCAR: hMN 41BBz; P2A; IL21; Furin E2AT2A; IL15 |
| SB08032 | aCAR: hMN 41BBz; P2A; HA-crIL15 (slow) |
| SB08033 | aCAR: hMN 41BBz; P2A; HA- crIL15 (fast) |
| SB08034 | aCAR: hMN 41BBz; P2A; crIL15 (slow); E2AT2A; IL21 |
| SB08035 | aCAR: hMN 41BBz; P2A; crIL15 (fast); E2AT2A; IL21 |
| SB08036 | aCAR: hMN14 CD28-2B4z; P2A; sIL15 |
| SB08037 | aCAR: hMN14 CD28-2B4z; P2A; IL21; Furin E2AT2A; |
| | IL15 |
| SB08038 | aCAR: hMN14 CD28-2B4z; P2A; HA-crIL15 (slow) |
| SB08039 | aCAR: hMN14 CD28-2B4z; P2A; HA-crIL15 (fast) |
| SB08040 | aCAR: hMN14 CD28-2B4z; P2A; crIL15 (slow); E2AT2A; |
| | IL21 |
| SB08041 | aCAR: hMN14 CD28-2B4z; P2A; crIL15 (fast); E2AT2A; |
| | IL21 |
| SB08042 | aCAR: hMN 28z; P2A-IL15 chimeric |
| SB08043 | aCAR: hMN OX40z; P2A-IL15 chimeric |
| SB08044 | aCAR: hMN 41BBz; P2A-IL15 chimeric |
| SB08045 | aCAR: hMN14 CD28-2B4z P2A-IL15 chimeric |
| SB04753 | iCAR: aVSIG2 H-Whitlow-L V5 CD8-LIR1-LIR1-P2A |
| | PuroR |
| SB05890, | aCAR: hMN 41BBz |
| SB05382 | aCAR: hMN 28z |

### Example 12: Engineered NK Cells Comprising CEA aCAR, VSIG2 iCAR, crIL15, IL21

The following multicistronic constructs were prepared with promoter and encoding sequences in the following orders (from 5′ to 3′).

| | | Component | Component | Component | Component |
|---|---|---|---|---|---|
| SB# and description | Promoter | 1 | 2 | 3 | 4 |
| 10010 | pSFFV | CEA aCAR | VISIG2 | crIL-15 | IL-21 |
| aCEA-28z 2A_C aVSIG2-SIRPa 2A_B crIL- | | | iCAR | | |
| 15 (+furin cleavage site) 2A_A IL-21 | | | | | |
| 10055 | pSFFV | crIL-15 | CEA aCAR | VISIG2 | IL-21 |
| crIL-15 2A_A aCEA-28z 2A_C aVSIG2- | | | | iCAR | |
| SIRPa 2A_B IL-21 | | | | | |
| 10063 | pSFFV | crIL-15 | IL-21 | CEA aCAR | VISIG2 |
| crIL-15 (+furin cleavage site) 2A_A IL-21 | | | | | iCAR |
| 2A_B aCEA-28z 2A_C aVSIG2-SIRPa | | | | | |
| 10064 | pSFFV | IL-21 | crIL-15 | CEA aCAR | VISIG2 |
| IL-21 (+furin clevage site) 2A_A crIL-15 | | | | | iCAR |
| 2A_B aCEA-28z 2A_C aVSIG2-SIRPa | | | | | |

Amino acid sequences of the above constructs are described herein, see Table below. Sequences of the 2A_A, 2A_B, and 2A_C linkers are provided herein.

| | SEQ ID NO: |
|---|---|
| Component | (AA) |
| aCEA-28Z aCAR | 362 |
| aVSIG2-SIRPa | 419 |
| crIL-15 | 355 |
| IL-21 | 359 |
| crIL-15 (+furin cleavage site) | 415 |
| IL-21 (+furin clevage site) | 413 |

### Retrovirus Production

DNA was transfected into GP2-293 (γ-retrovirus) producer cells following manufacturer recommendations. Viral supernatant was collected, clarified by centrifugation, treated by MgCl2 and Benzonase, and concentrated using Lenti-X concentrator.

### NK Cell Engineering

Primary NK cells were isolated from PBMCs from healthy donors and frozen in liquid nitrogen. For individual experiments, single vials of frozen NK cells were thawed and stimulated with irradiated feeder cells (engineered K562 cells). NK cells were expanded in 6-well G-Rex plates in NK media (NK MACS media with 5% human AB serum with 100 U/mL IL2). For virus transduction preparation, 12-well plates were coated with recombinant human fibronectin fragment (RetroNectin) according to manufacturer protocols. NK cells and retrovirus were added to coated plates and centrifuge at 1000 g for 2 hours at 32° C. After transduction, the NK cells were transferred to 12-well G-Rex for expansion. After 7-14 days, expression was checked by flow cytometry and cells were harvested for use in assays. Vector copy number (VCN) was determined by qPCR against woodchuck hepatitis virus post-transcriptional regulatory element (WPRE) within the construct. The secreted cytokines, IL-15 and IL-21 were quantified using Luminex assays (multiplexed ELISA assays). 5×105 NK cells were plated in 96-well plate in a total volume of 200 μL of medium. After 24 hours, plates were centrifuged and 100 μL supernatant were collected. The secreted cytokines were measured using customized MILLIPLEX multiplex assay kit and Luminex MAGPIX System according to manufacturer protocols.

### Flow Cytometry

For aCAR-Myc, iCAR-V5 detection and IL-15 detection, Myc-tag antibody (Cell Signaling Technology), V5-tag antibody (ThermoFisher), anti-human IL-15 antibody (BioLegend) and Sytox Blue (Life Technologies) were used to stain the transduced NK cells. For staining preparation, transduced NK cells were washed twice with FACS buffer, then stained with antibodies and viability dye for 1 hour in 4° C. After incubation, cells were washed and resuspended in an appropriate volume of FACS buffer for flow cytometry (Beckman CytoFLEX).

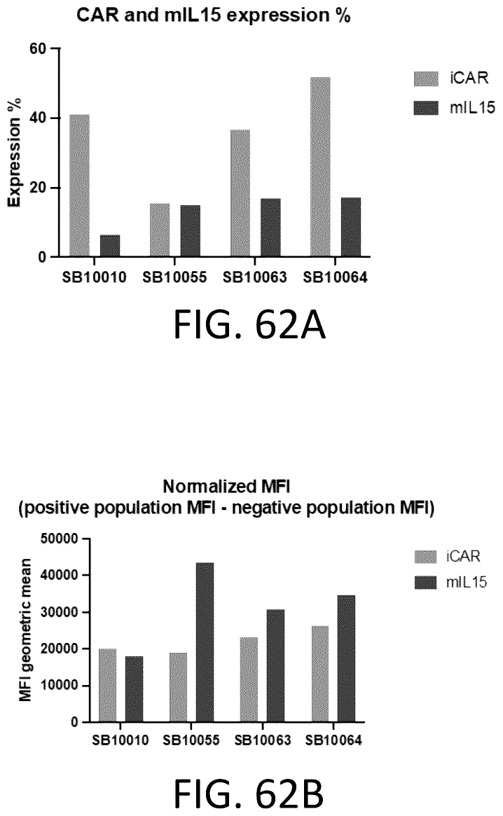

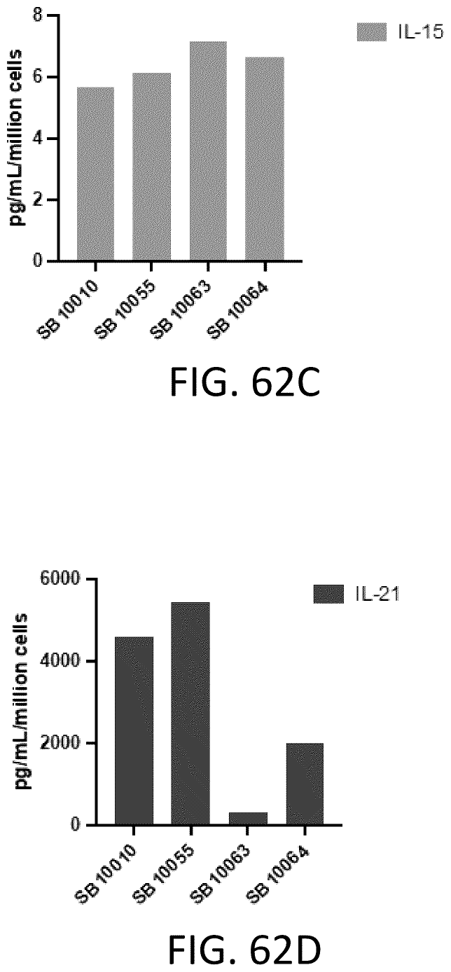

Surface iCAR and IL15 expression results by flow cytometry are shown in FIG. 62A. As shown, surface VSIG2 iCAR and crIL15 is detected in an appreciable percentage of cells engineered with the four constructs.

FIG. 62B shows normalized MFI for NK cells transduced with all four constructs. As shown, appreciable iCAR and crIL15 levels are detected in the positive populations of cells engineered with the four constructs.

FIGS. 62C and 62D show levels of secreted IL-15 and IL-21. As shown, cells engineered with the four constructs all exhibited detectable levels of secreted IL-15 and IL-21.

Viral copy number was determined by qPCR against woodchuck hepatitis virus post-transcriptional regulatory element (WPRE) within the construct. VCN results are shown in the Table below.

| SB | Average VCN |
|---|---|
| 10010 | 2.6 |
| 10055 | 0.9 |
| 10063 | 2.2 |
| 10064 | 2.3 |

Taken together, all four constructs successfully expressed the four component payloads in engineered NK cells. Constructs with the following multicistronic configuration IL21_crIL15_aCAR_iCAR exhibited the highest CAR and surface IL15 expression.

### Example 13: Further Testing of Engineered NK Cells Comprising CEA aCAR, VSIG2 iCAR, crIL15, IL21

Multicistronic constructs for encoding an aCAR (e.g., an aCAR targeting CEACAM5), an iCAR (e.g., an iCAR targeting VSIG2), a membrane-cleavable chimeric protein (e.g., crIL15), and a cytokine (e.g., IL21) are prepared. Exemplary multicistronic constructs for encoding a CEACAM5 aCAR, a VSIG2 iCAR, crIL15, and IL21 are described herein, e.g., SB10010, 10055, 10063, and 10064. Components of exemplary multicistronic constructs are described, e.g., in Example 13.

Another exemplary multicistronic construct is prepared expressing the following components: IL-21 (+furin cleavage site)-2A_A linker-crIL-15 (no G4S linker(SEQ ID NO:444) or tag)-2A_B linker-aCEA-28z (G4S linker(SEQ ID NO:444), no tag)-2A_C linker-aVSIG2-SIRPα (no tag). Amino acid sequences of this construct is described herein, see Table below. Sequences of the 2A_A, 2A_B, and 2A_C linkers are provided herein.

| | SEQ ID NO: |
|---|---|
| Component | (AA) |
| aCEA-28Z aCAR (G4S linker(SEQ ID NO: 444), no tag) | 362 |
| aVSIG2-SIRPa (no tag) | 359 |
| crIL-15 (no G4S linker (SEQ ID NO: 444) or tag) | 410 |
| IL-21 (+furin cleavage site) | 413 |

### Retrovirus Production

DNA is transfected into GP2-293 (γ-retrovirus) producer cells following manufacturer recommendations. Viral supernatant is collected, clarified by centrifugation, treated by MgCl2 and Benzonase, and concentrated using Lenti-X concentrator.

### NK Cell Engineering

Primary NK cells are isolated from PBMCs from healthy donors and frozen in liquid nitrogen. For individual experiments, single vials of frozen NK cells are thawed and stimulated with irradiated feeder cells (engineered K562 cells). NK cells are expanded in 6-well G-Rex plates in NK media (NK MACS media with 5% human AB serum with 100 U/mL IL2). For virus transduction preparation, 12-well plates are coated with recombinant human fibronectin fragment (RetroNectin) according to manufacturer protocols. NK cells and retrovirus are added to coated plates and centrifuge at 1000 g for 2 hours at 32° C. After transduction, the NK cells are transferred to 12-well G-Rex for expansion. After 7-14 days, expression is checked by flow cytometry and cells are harvested for use in assays. Vector copy number (VCN) is determined by qPCR against woodchuck hepatitis virus post-transcriptional regulatory element (WPRE) within the construct. The secreted cytokines, IL-15 and IL-21 are quantified using Luminex assays (multiplexed ELISA assays). 5×105 NK cells are plated in 96-well plate in a total volume of 200 μL of medium. After 24 hours, plates are centrifuged and 100 μL supernatant are collected. The secreted cytokines are measured using customized MILLIPLEX multiplex assay kit and Luminex MAGPIX System according to manufacturer protocols.

### Flow Cytometry

Cells are stained with an antibody cocktail to detect the engineered payloads and determine viability. Payload detection for myc or V5 tagged aCAR and iCAR constructs is performed according to methods described in Example 13.

Payload detection of “no tag” versions of the aCAR and iCAR is performed as follows: To detect the aCAR, a fluorochrome-conjugated anti-G4S Linker(SEQ ID NO:444) (E702V) Rabbit mAb from Cell Signaling Technologies is used. To detect the iCAR, a fluorochrome-conjugated anti-Whitlow/218 Linker (E3U7Q) Rabbit mAb from Cell Signaling Technologies is used. To detect residual uncleaved crIL-15, a fluorochrome-conjugated anti-human IL-15 mAb from BioLegend is used. To assess cell viability, Sytox (Life Technologies) is used to stain the transduced NK cells. For staining preparation, transduced NK cells are washed twice with FACS buffer, then stained with antibodies and viability dye for 1 hour in 4° C. After incubation, cells are washed and resuspended in an appropriate volume of FACS buffer for flow cytometry (Beckman CytoFLEX).

Furthermore, killing function of NK cells engineered with the constructs are evaluated in a mixed target serial killing assay. For each round of killing, two engineered DLD-1 (colorectal adenocarcinoma) cell lines are mixed at 1:1 ratio and pre-plated in flat-bottom tissue culture plates. The TA+PA− DLD-1 target cell line is engineered to express: GFP reporter protein, a CEA target antigen that is recognized by the aCAR (TA+), and an off-target protective antigen Her2 that is not recognized by the iCAR (PA−). The TA+PA+ DLD-1 target cell line is engineered to express: mCherry reporter, the same CEA target antigen recognized by the aCAR (TA+), and on-target protective antigen VSIG2 recognized by the iCAR (PA+).

Effector cells (unengineered NK cells (NV) or engineered NK cells) are added to pre-plated DLD-1 target cells at E:T ratio of 1:4. (For clarity, E:T ratio refers to the ratio of Effector Cells (NK cells) to the pre-plated DLD-1 target cells.) After approximately 45 hours post co-culture, NK cells in suspension are transferred to new plated target cells for next round of killing, for total of three rounds of killing. Images of the assay wells are automatically taken at four-hour intervals on the Incucyte. Killing of the two engineered DLD-1 cell lines as are quantified as counts of reporter protein positive cells over time.

It is expected that NK cells engineered with the multicistronic constructs will express detectable levels of the aCAR, iCAR, and crIL15, as measured by flow cytometry, and will also exhibit detectable levels of secreted IL15 and IL21, as measured by Luminex assay. It is also expected that engineered NK cells will exhibit killing of the TA+PA− DLD-1 target cell line.

### Additional Sequences

| Construct | |
|---|---|
| # | Full payload sequence (AA) |
| SB10010 | MALPVTALLLPLALLLHAARPDIQLTQSPSSLSASVGDRVTITCKASQDVGTSVAWYQQKPGKAPKLLIYW |
| | TSTRHTGVPSRFSGSGSGTDFTFTISSLQPEDIATYYCQQYSLYRSFGQGTKVEIKGGSGSGGSGSGGSGSEV |
| | QLVESGGGVVQPGRSLRLSCSASGFDFTTYWMSWVRQAPGKGLEWIGEIHPDSSTINYAPSLKDRFTISRDN |
| | AKNTLFLQMDSLRPEDTGVYFCASLYFGFPWFAYWGQGTPVTVSSEQKLISEEDLNGAATTTPAPRPPTPA |
| | PTIALQPLSLRPEACRPAAGGAVHTRGLDFACDFWVLVVVGGVLACYSLLVTVAFIIFWVRSKRSRLLHSD |
| | YMNMTPRRPGPTRKHYQPYAPPRDFAAYRSRVKFSRSADAPAYKQGQNQLYNELNLGRREEYDVLDKRR |
| | GRDPEMGGKPRRKNPQEGLYNELQKDKMAEAYSEIGMKGERRRGKGHDGLYQGLSTATKDTYDALHMQ |
| | ALPPRGSGQCTNYALLKLAGDVESNPGPGSGEGRGSLLTCGDVEENPGPMALPVTALLLPLALLLHAARPD |
| | IQMTQSPASLSASVGETVTMTCRASENIYSYLAWYQQKQGKSPQLLVFNAETLPEGVPSRFSGTGSGTHFSL |
| | RINSLQPEDFGSYYCQHHYVIPWTFGGGTKLEIKGSTSGSGKPGSGEGSTKGEVQMVESGGDLVKPGGSLK |
| | LSCAASGFTFSNSGMSWVRQTPDKRLEWVASISDGGLYTHYPDSVKGRFTISRDNGKSTLYLQMSSLRSED |
| | TAIYYCARQGVRPFFDYWGQGTTLTVSSGKPIPNPLLGLDSTNGAATTTPAPRPPTPAPTIALQPLSLRPEAC |
| | RPAAGGAVHTRGLDFACDIVVGVVCTLLVALLMAALYLVRIRQKKAQGSTSSTRLHEPEKNAREITQDTN |
| | DITYADLNLPKGKKPAPQAAEPNNHTEYASIQTSPQPASEDTLTYADLDMVHLNRTPKQPAPKPEPSFSEYA |
| | SVQVPRKGSGQCTNYALLKLAGDVESNPGPGSGEGRGSLLTCGDVEENPGPMDWTWILFLVAAATRVHS |
| | YPYDVPDYAGGGGSNWVNVISDLKKIEDLIQSMHIDATLYTESDVHPSCKVTAMKCFLLELQVISLESGDA |
| | SIHDTVENLIILANNSLSSNGNVTESGCKECEELEEKNIKEFLQSFVHIVQMFINTSSGGGGSGGGGSGVTPEP |
| | IFSLIGGGSGGGGSGGGSLQLLPSWAITLISVNGIFVICCLTYCFAPRCRERRRNERLRRESVRPVRRKRGSGQ |
| | CTNYALLKLAGDVESNPGPGSGEGRGSLLTCGDVEENPGPMERIVICLMVIFLGTLVHKSSSQGQDRHMIR |
| | MRQLIDIVDQLKNYVNDLVPEFLPAPEDVETNCEWSAFSCFQKAQLKSANTGNNERIINVSIKKLKRKPPST |
| | NAGRRQKHRLTCPSCDSYEKKPPKEFLERFKSLLQKMIHQHLSSRTHGSEDS (SEQ ID NO: 405) |
| SB10055 | MDWTWILFLVAAATRVHSYPYDVPDYAGGGGSNWVNVISDLKKIEDLIQSMHIDATLYTESDVHPSCKVT |
| | AMKCFLLELQVISLESGDASIHDTVENLIILANNSLSSNGNVTESGCKECEELEEKNIKEFLQSFVHIVQMFIN |
| | TSSGGGGSGGGGSGVTPEPIFSLIGGGSGGGGSGGGSLQLLPSWAITLISVNGIFVICCLTYCFAPRCRERRRN |
| | ERLRRESVRPVGSGQCTNYALLKLAGDVESNPGPGSGEGRGSLLTCGDVEENPGPMALPVTALLLPLALLL |
| | HAARPDIQLTQSPSSLSASVGDRVTITCKASQDVGTSVAWYQQKPGKAPKLLIYWTSTRHTGVPSRFSGSGS |
| | GTDFTFTISSLQPEDIATYYCQQYSLYRSFGQGTKVEIKGGSGSGGSGSGGSGSEVQLVESGGGVVQPGRSL |
| | RLSCSASGFDFTTYWMSWVRQAPGKGLEWIGEIHPDSSTINYAPSLKDRFTISRDNAKNTLFLQMDSLRPED |
| | TGVYFCASLYFGFPWFAYWGQGTPVTVSSEQKLISEEDLNGAATTTPAPRPPTPAPTIALQPLSLRPEACRPA |
| | AGGAVHTRGLDFACDFWVLVVVGGVLACYSLLVTVAFIIFWVRSKRSRLLHSDYMNMTPRRPGPTRKHY |
| | QPYAPPRDFAAYRSRVKFSRSADAPAYKQGQNQLYNELNLGRREEYDVLDKRRGRDPEMGGKPRRKNPQ |
| | EGLYNELQKDKMAEAYSEIGMKGERRRGKGHDGLYQGLSTATKDTYDALHMQALPPRGSGQCTNYALL |
| | KLAGDVESNPGPGSGEGRGSLLTCGDVEENPGPMALPVTALLLPLALLLHAARPDIQMTQSPASLSASVGE |
| | TVTMTCRASENIYSYLAWYQQKQGKSPQLLVFNAETLPEGVPSRFSGTGSGTHFSLRINSLQPEDFGSYYCQ |
| | HHYVIPWTFGGGTKLEIKGSTSGSGKPGSGEGSTKGEVQMVESGGDLVKPGGSLKLSCAASGFTFSNSGMS |
| | WVRQTPDKRLEWVASISDGGLYTHYPDSVKGRFTISRDNGKSTLYLQMSSLRSEDTAIYYCARQGVRPFFD |
| | YWGQGTTLTVSSGKPIPNPLLGLDSTNGAATTTPAPRPPTPAPTIALQPLSLRPEACRPAAGGAVHTRGLDF |
| | ACDIVVGVVCTLLVALLMAALYLVRIRQKKAQGSTSSTRLHEPEKNAREITQDTNDITYADLNLPKGKKPA |
| | PQAAEPNNHTEYASIQTSPQPASEDTLTYADLDMVHLNRTPKQPAPKPEPSFSEYASVQVPRKGSGQCTNY |
| | ALLKLAGDVESNPGPGSGEGRGSLLTCGDVEENPGPMERIVICLMVIFLGTLVHKSSSQGQDRHMIRMRQLI |
| | DIVDQLKNYVNDLVPEFLPAPEDVETNCEWSAFSCFQKAQLKSANTGNNERIINVSIKKLKRKPPSTNAGRR |
| | QKHRLTCPSCDSYEKKPPKEFLERFKSLLQKMIHQHLSSRTHGSEDS (SEQ ID NO: 406) |
| SB10063 | MDWTWILFLVAAATRVHSYPYDVPDYAGGGGSNWVNVISDLKKIEDLIQSMHIDATLYTESDVHPSCKVT |
| | AMKCFLLELQVISLESGDASIHDTVENLIILANNSLSSNGNVTESGCKECEELEEKNIKEFLQSFVHIVQMFIN |
| | TSSGGGGSGGGGSGVTPEPIFSLIGGGSGGGGSGGGSLQLLPSWAITLISVNGIFVICCLTYCFAPRCRERRRN |
| | ERLRRESVRPVRRKRGSGQCTNYALLKLAGDVESNPGPGSGEGRGSLLTCGDVEENPGPMERIVICLMVIFL |
| | GTLVHKSSSQGQDRHMIRMRQLIDIVDQLKNYVNDLVPEFLPAPEDVETNCEWSAFSCFQKAQLKSANTG |
| | NNERIINVSIKKLKRKPPSTNAGRRQKHRLTCPSCDSYEKKPPKEFLERFKSLLQKMIHQHLSSRTHGSEDSG |
| | SGQCTNYALLKLAGDVESNPGPGSGEGRGSLLTCGDVEENPGPMALPVTALLLPLALLLHAARPDIQLTQS |
| | PSSLSASVGDRVTITCKASQDVGTSVAWYQQKPGKAPKLLIYWTSTRHTGVPSRFSGSGSGTDFTFTISSLQ |
| | PEDIATYYCQQYSLYRSFGQGTKVEIKGGSGSGGSGSGGSGSEVQLVESGGGVVQPGRSLRLSCSASGFDFT |
| | TYWMSWVRQAPGKGLEWIGEIHPDSSTINYAPSLKDRFTISRDNAKNTLFLQMDSLRPEDTGVYFCASLYF |
| | GFPWFAYWGQGTPVTVSSEQKLISEEDLNGAATTTPAPRPPTPAPTIALQPLSLRPEACRPAAGGAVHTRGL |
| | DFACDFWVLVVVGGVLACYSLLVTVAFIIFWVRSKRSRLLHSDYMNMTPRRPGPTRKHYQPYAPPRDFAA |
| | YRSRVKFSRSADAPAYKQGQNQLYNELNLGRREEYDVLDKRRGRDPEMGGKPRRKNPQEGLYNELQKDK |
| | MAEAYSEIGMKGERRRGKGHDGLYQGLSTATKDTYDALHMQALPPRGSGQCTNYALLKLAGDVESNPGP |
| | GSGEGRGSLLTCGDVEENPGPMALPVTALLLPLALLLHAARPDIQMTQSPASLSASVGETVTMTCRASENI |
| | YSYLAWYQQKQGKSPQLLVFNAETLPEGVPSRFSGTGSGTHFSLRINSLQPEDFGSYYCQHHYVIPWTFGG |
| | GTKLEIKGSTSGSGKPGSGEGSTKGEVQMVESGGDLVKPGGSLKLSCAASGFTFSNSGMSWVRQTPDKRLE |
| | WVASISDGGLYTHYPDSVKGRFTISRDNGKSTLYLQMSSLRSEDTAIYYCARQGVRPFFDYWGQGTTLTVS |
| | SGKPIPNPLLGLDSTNGAATTTPAPRPPTPAPTIALQPLSLRPEACRPAAGGAVHTRGLDFACDIVVGVVCTL |
| | LVALLMAALYLVRIRQKKAQGSTSSTRLHEPEKNAREITQDTNDITYADLNLPKGKKPAPQAAEPNNHTEY |
| | ASIQTSPQPASEDTLTYADLDMVHLNRTPKQPAPKPEPSFSEYASVQVPRK (SEQ ID NO: 407) |
| SB10064 | MERIVICLMVIFLGTLVHKSSSQGQDRHMIRMRQLIDIVDQLKNYVNDLVPEFLPAPEDVETNCEWSAFSCF |
| | QKAQLKSANTGNNERIINVSIKKLKRKPPSTNAGRRQKHRLTCPSCDSYEKKPPKEFLERFKSLLQKMIHQH |
| | LSSRTHGSEDSRRKRGSGQCTNYALLKLAGDVESNPGPGSGEGRGSLLTCGDVEENPGPMDWTWILFLVA |
| | AATRVHSYPYDVPDYAGGGGSNWVNVISDLKKIEDLIQSMHIDATLYTESDVHPSCKVTAMKCFLLELQVI |
| | SLESGDASIHDTVENLIILANNSLSSNGNVTESGCKECEELEEKNIKEFLQSFVHIVQMFINTSSGGGGSGGG |
| | GSGVTPEPIFSLIGGGSGGGGSGGGSLQLLPSWAITLISVNGIFVICCLTYCFAPRCRERRRNERLRRESVRPV |
| | GSGQCTNYALLKLAGDVESNPGPGSGEGRGSLLTCGDVEENPGPMALPVTALLLPLALLLHAARPDIQLTQ |
| | SPSSLSASVGDRVTITCKASQDVGTSVAWYQQKPGKAPKLLIYWTSTRHTGVPSRFSGSGSGTDFTFTISSL |
| | QPEDIATYYCQQYSLYRSFGQGTKVEIKGGSGSGGSGSGGSGSEVQLVESGGGVVQPGRSLRLSCSASGFD |
| | FTTYWMSWVRQAPGKGLEWIGEIHPDSSTINYAPSLKDRFTISRDNAKNTLFLQMDSLRPEDTGVYFCASL |
| | YFGFPWFAYWGQGTPVTVSSEQKLISEEDLNGAATTTPAPRPPTPAPTIALQPLSLRPEACRPAAGGAVHTR |
| | GLDFACDFWVLVVVGGVLACYSLLVTVAFIIFWVRSKRSRLLHSDYMNMTPRRPGPTRKHYQPYAPPRDF |
| | AAYRSRVKFSRSADAPAYKQGQNQLYNELNLGRREEYDVLDKRRGRDPEMGGKPRRKNPQEGLYNELQK |
| | DKMAEAYSEIGMKGERRRGKGHDGLYQGLSTATKDTYDALHMQALPPRGSGQCTNYALLKLAGDVESN |
| | PGPGSGEGRGSLLTCGDVEENPGPMALPVTALLLPLALLLHAARPDIQMTQSPASLSASVGETVTMTCRAS |
| | ENIYSYLAWYQQKQGKSPQLLVFNAETLPEGVPSRFSGTGSGTHFSLRINSLQPEDFGSYYCQHHYVIPWTF |
| | GGGTKLEIKGSTSGSGKPGSGEGSTKGEVQMVESGGDLVKPGGSLKLSCAASGFTFSNSGMSWVRQTPDK |
| | RLEWVASISDGGLYTHYPDSVKGRFTISRDNGKSTLYLQMSSLRSEDTAIYYCARQGVRPFFDYWGQGTTL |
| | TVSSGKPIPNPLLGLDSTNGAATTTPAPRPPTPAPTIALQPLSLRPEACRPAAGGAVHTRGLDFACDIVVGVV |
| | CTLLVALLMAALYLVRIRQKKAQGSTSSTRLHEPEKNAREITQDTNDITYADLNLPKGKKPAPQAAEPNNH |
| | TEYASIQTSPQPASEDTLTYADLDMVHLNRTPKQPAPKPEPSFSEYASVQVPRK (SEQ ID NO: 408) |
| Payload | MERIVICLMVIFLGTLVHKSSSQGQDRHMIRMRQLIDIVDQLKNYVNDLVPEFLPAPEDVETNCEWSAFSCF |
| (with | QKAQLKSANTGNNERIINVSIKKLKRKPPSTNAGRRQKHRLTCPSCDSYEKKPPKEFLERFKSLLQKMIHQH |
| tagless | LSSRTHGSEDSRRKRGSGQCTNYALLKLAGDVESNPGPGSGEGRGSLLTCGDVEENPGPMDWTWILFLVA |
| aCAR and | AATRVHSNWVNVISDLKKIEDLIQSMHIDATLYTESDVHPSCKVTAMKCFLLELQVISLESGDASIHDTVEN |
| iCAR) | LIILANNSLSSNGNVTESGCKECEELEEKNIKEFLQSFVHIVQMFINTSGSGQCTNYALLKLAGDVESNPGPG |
| | SGEGRGSLLTCGDVEENPGPMALPVTALLLPLALLLHAARPDIQLTQSPSSLSASVGDRVTITCKASQDVGT |
| | SVAWYQQKPGKAPKLLIYWTSTRHTGVPSRFSGSGSGTDFTFTISSLQPEDIATYYCQQYSLYRSFGQGTKV |
| | EIKGGGGSGGGGSGGGGSEVQLVESGGGVVQPGRSLRLSCSASGFDFTTYWMSWVRQAPGKGLEWIGEIH |
| | PDSSTINYAPSLKDRFTISRDNAKNTLFLQMDSLRPEDTGVYFCASLYFGFPWFAYWGQGTPVTVSSNGAA |
| | TTTPAPRPPTPAPTIALQPLSLRPEACRPAAGGAVHTRGLDFACDFWVLVVVGGVLACYSLLVTVAFIIFWV |
| | RSKRSRLLHSDYMNMTPRRPGPTRKHYQPYAPPRDFAAYRSRVKFSRSADAPAYKQGQNQLYNELNLGR |
| | REEYDVLDKRRGRDPEMGGKPRRKNPQEGLYNELQKDKMAEAYSEIGMKGERRRGKGHDGLYQGLSTA |
| | TKDTYDALHMQALPPRGSGQCTNYALLKLAGDVESNPGPGSGEGRGSLLTCGDVEENPGPMALPVTALLL |
| | PLALLLHAARPDIQMTQSPASLSASVGETVTMTCRASENIYSYLAWYQQKQGKSPQLLVFNAETLPEGVPS |
| | RFSGTGSGTHFSLRINSLQPEDFGSYYCQHHYVIPWTFGGGTKLEIKGSTSGSGKPGSGEGSTKGEVQMVES |
| | GGDLVKPGGSLKLSCAASGFTFSNSGMSWVRQTPDKRLEWVASISDGGLYTHYPDSVKGRFTISRDNGKST |
| | LYLQMSSLRSEDTAIYYCARQGVRPFFDYWGQGTTLTVSSNGAATTTPAPRPPTPAPTIALQPLSLRPEACR |
| | PAAGGAVHTRGLDFACDIVVGVVCTLLVALLMAALYLVRIRQKKAQGSTSSTRLHEPEKNAREITQDTNDI |
| | TYADLNLPKGKKPAPQAAEPNNHTEYASIQTSPQPASEDTLTYADLDMVHLNRTPKQPAPKPEPSFSEYAS |
| | VQVPRK (SEQ ID NO: 409) |

### Interpretations

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A multicistronic expression system comprising:
(a) an exogenous polynucleotide sequence encoding a first cytokine;
(b) an exogenous polynucleotide sequence encoding a second cytokine; and
(c) an exogenous polynucleotide sequence encoding an activating chimeric antigen receptor (aCAR), optionally wherein the aCAR comprises: (i) a first antigen-binding domain, (ii) one or more intracellular signaling domains that stimulate an immune response, and (iii) one or more polypeptides selected from the group consisting of: a signal peptide, a transmembrane domain, a hinge domain, a spacer region, one or more peptide linkers, and combinations thereof; and
(d) an exogenous polynucleotide sequence encoding an inhibitory CAR (iCAR),
wherein each exogenous polynucleotide sequence comprises a 5′ end and a 3′ end.

2. A multicistronic expression system comprising:
(a) an exogenous polynucleotide sequence encoding a first cytokine;
(b) an exogenous polynucleotide sequence encoding a second cytokine; and
(c) an exogenous polynucleotide sequence encoding an activating chimeric antigen receptor (aCAR),
wherein each exogenous polynucleotide sequence comprises a 5′ end and a 3′ end, and
wherein the aCAR comprises:
(i) a first antigen-binding domain that binds to a target selected from: CEA, CEACAM1, CEACAM5, and CEACAM6, optionally wherein the first antigen-binding domain of the aCAR binds CEACAM5, optionally wherein the first antigen binding domain of the aCAR comprises the amino acid sequence set forth in SEQ ID NO: 381;
(ii) one or more intracellular signaling domains that stimulate an immune response; and
(iii) one or more polypeptides selected from the group consisting of: a signal peptide, a transmembrane domain, a hinge domain, a spacer region, one or more peptide linkers, and combinations thereof.

3. The multicistronic expression system of any one of the preceding claims, wherein:
(i) the one or more intracellular signaling domains of the aCAR are selected from the group consisting of: CD3-zeta, FcR gamma, FcR beta, CD3 gamma, CD3 delta, CD3 epsilon, CD5, CD22, CD79a, CD79b, CD278, FcεRI, DAP10, DAP12, CD66d, CD97, CD2, ICOS, CD27, CD154, CD8, OX40, 4-1BB, CD28, ZAP40, CD30, GITR, HVEM, DAP10, DAP12, MyD88, 2B4, CD40, PD-1, LFA-1, CD7, LIGHT, NKG2C, B7-H3, an MHC class I molecule, a TNF receptor protein, an Immunoglobulin-like protein, a cytokine receptor, an integrin, a SLAM protein, an activating NK cell receptor, BTLA, a Toll ligand receptor, CDS, ICAM-1, (CD11a/CD18), BAFFR, KIRDS2, SLAMF7, NKp80 (KLRF1), NKp44, NKp30, NKp46, CD19, CD4, IL2R beta, IL2R gamma, IL7R alpha, ITGA4, VLAl, CD49a, ITGA4, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and combinations thereof, and/or
(ii) the aCAR comprises a hinge domain selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof, and/or
(iii) the aCAR comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, and BTLA, and/or
(iv) the aCAR comprises a signal peptide selected from the group consisting of: IgE, IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, CXCL12, IL21, CD8, NKG2D, TNFR2, GMCSF, and GM-CSFRa.

4. The multicistronic expression system of claim 1 or 3, wherein the iCAR comprises:
(a) a second antigen-binding domain;
(b) one or more intracellular signaling domains that inhibit an immune response; and
(c) one or more polypeptides selected from the group consisting of: a signal peptide, a transmembrane domain, a hinge domain, a spacer region, one or more peptide linkers, and combinations thereof.

5. The multicistronic expression system of claim 4, wherein the second antigen-binding domain of the iCAR binds VSIG2, optionally wherein:
(i) the iCAR comprises an LIR1 intracellular inhibitory domain, optionally wherein the intracellular inhibitory domain comprises the amino acid sequence set forth in SEQ ID NO: 387, or
(ii) the iCAR comprises an SIRPα intracellular inhibitory domain, optionally wherein the intracellular inhibitory domain comprises the amino acid sequence set forth in SEQ ID NO: 385.

6. The multicistronic expression system of claim 4 or 5, wherein:
(i) the iCAR comprises a hinge domain selected from the group consisting of a human Ig (immunoglobulin) hinge, an IgG4 hinge, an IgG2 hinge, a CD8a hinge, or an IgD hinge, a KIR2DS2 hinge, an LNGFR hinge, a LIR1 hinge, a PDGFR-beta extracellular linker, and combinations thereof, and/or
(ii) the iCAR comprises a transmembrane domain selected from the group consisting of: PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, B7-1, LIR1, SIRPα, and BTLA, and/or
(iii) the iCAR comprises a signal peptide selected from the group consisting of: IgE, IL12, IL2, optimized IL2, trypsiongen-2, Gaussia luciferase, CD5, human IgKVII, murine IgKVII, VSV-G, prolactin, serum albumin preprotein, azurocidin preprotein, osteonectin, CD33, IL6, IL8, CCL2, TIMP2, VEGFB, osteoprotegerin, serpin E1, GROalpha, CXCL12, IL21, CD8, NKG2D, TNFR2, GMCSF, and GM-CSFRa.

7. The multicistronic expression system of any one of the preceding claims, wherein:
(i) the exogenous polynucleotide encoding the first cytokine, the exogenous polynucleotide encoding the second cytokine, the exogenous polynucleotide encoding the aCAR, and the exogenous polynucleotide encoding the iCAR are comprised within a single expression vector, or
(ii) the exogenous polynucleotide encoding the first cytokine, the exogenous polynucleotide encoding the second cytokine, and the exogenous polynucleotide encoding the aCAR are comprised within a first expression vector, and the exogenous polynucleotide encoding the iCAR is comprised within a second expression vector.

8. The multicistronic expression system of any one of the preceding claims, further comprising ribosome skipping sites between each exogenous polynucleotide.

9. The multicistronic expression system of any one of claims 1-8, wherein at least one of the first and the second cytokines is a controlled release cytokine having the formula: 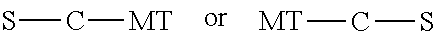
wherein
S comprises a secretable effector molecule;
C comprises a protease cleavage site; and
MT comprises a cell membrane tethering domain.

10. The multicistronic expression system of claim 9, wherein:
(i) the protease cleavage site is cleaved by ADAM10 and/or ADAM17, and/or
(ii) the protease cleavage site comprises the amino acid sequence set forth in SEQ ID NO: 180 or SEQ ID NO: 191, and/or
(iii) the cell membrane tethering domain comprises a transmembrane domain selected from the group consisting of: B7-1, PDGFR-beta, CD8, CD28, CD3zeta-chain, CD4, 4-1BB, OX40, ICOS, CTLA-4, PD-1, LAG-3, 2B4, LNGFR, NKG2D, EpoR, TNFR2, LIR1, and BTLA, optionally wherein the cell membrane tethering domain comprises a B7-1 transmembrane domain comprising the amino acid sequence set forth in SEQ ID NO: 219.

11. The multicistronic expression system of any one of the preceding claims, wherein:
(i) the first cytokine is IL15, optionally wherein the IL15 comprises the amino acid sequence set forth in SEQ ID NO: 285, or optionally wherein the IL15 is controlled-release IL15 (crIL15), and/or
(ii) the second cytokine is IL21, optionally wherein the IL21 comprises the amino acid sequence set forth in SEQ ID NO: 360, or optionally wherein the IL21 is controlled-release IL21 (crIL21), and/or
(iii) the first or second cytokine comprises an amino acid sequence set forth in any one of SEQ ID NOs: 355-359, 361, and 391, and/or
(iv) the first or second cytokine is encoded by a nucleic acid sequence set forth in any one of SEQ ID NOs: 367-372, and 392.

12. A multicistronic expression system comprising:
(a) an exogenous polynucleotide sequence encoding a first cytokine;
(b) an exogenous polynucleotide sequence encoding a second cytokine; and
(c) an exogenous polynucleotide sequence encoding a chimeric antigen receptor (CAR),
wherein each exogenous polynucleotide sequence comprises a 5′ end and a 3′ end.

13. An engineered cell comprising the multicistronic expression system of any one of claims 1-12.

14. The engineered cell of claim 13, wherein the engineered cell is an immune cell, optionally wherein the engineered cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell, optionally wherein the engineered cell is an NK cell.

15. A pharmaceutical composition comprising the engineered cell of claim 13 or 14, and a pharmaceutically acceptable carrier.

16. A method of treating a disease in a subjected in needed thereof, the method comprising administering a therapeutically effective dose of the engineered cell of claim 13 or 14 or the pharmaceutical composition of claim 15 to the subject, optionally wherein:
(i) the disease is a cancer, and/or
(ii) the isolated cell is allogenic to the subject or autologous to the subject.

17. A method of manufacturing an engineered cell, the method comprising transducing an isolated cell with the multicistronic expression system of any one of claims 1-12, optionally wherein:
(i) the isolated cell is an immune cell, and/or
(ii) the isolated cell is selected from the group consisting of a T cell, a Natural Killer (NK) cell, a cytotoxic T lymphocyte (CTL), a regulatory T cell, a Natural Killer T (NKT) cell, a myeloid cell, a macrophage, a human embryonic stem cell (ESC), an ESC-derived cell, a pluripotent stem cell, and induced pluripotent stem cell (iPSC), and an iPSC-derived cell, optionally wherein the isolated cell is an NK cell.
